(19) United States
(12) Patent Application Publication
(10) Pub. No.: US 2025/0346649 A1
(43) Pub. Date: Nov. 13, 2025

(54) ZIP CYTOKINE RECEPTORS

(71) Applicant: St. Jude Children's Research Hospital, Inc., Memphis, TN (US)

(72) Inventors: Stephen GOTTSCHALK, Germantown, TN (US); Giedre KRENCIUTE, Memphis, TN (US); Matthew BELL, Memphis, TN (US); Alejandro Allo ANIDO, Memphis, TN (US); Madanbabu Mohan, Memphis, TN (US); Besian SEJDIU, Memphis, TN (US)

(73) Assignee: St. Jude Children's Research Hospital, Inc., Memphis, TN (US)

(21) Appl. No.: 18/707,438

(22) PCT Filed: Nov. 4, 2022
(86) PCT No.: PCT/US2022/079291

§ 371 Date: May 3, 2024

Related U.S. Application Data
(60) Provisional application No. 63/263,637, filed on Nov. 5, 2021.

(51) Int. Cl. C07K 14/715; A61K 35/17; A61K 40/11; A61K 40/31; A61P 35/00; A61P 37/04; C12N 5/0783; C12N 15/86

(52) U.S. Cl. CPC C07K 14/7155; A61K 35/17; A61K 40/11; A61K 40/31; A61P 35/00; A61P 37/04; C07K 14/7153; C12N 5/0636; C12N 15/86; A61K 2239/15; A61K 2239/17; A61K 2239/21; A61K 2239/22; C07K 2319/40; C12N 2510/00; C12N 2740/16043

(57) ABSTRACT

The application relates to chimeric cytokine receptors, particularly chimeric cytokine receptors comprising one or more leucine zipper motifs, and their uses in tumor immunotherapy (e.g., adoptive cell therapy). The application further relates to methods of genetically modifying therapeutic immune cells resulting in an enhanced immune response against a target antigen. The application further relates to therapeutic cells that express said chimeric cytokine receptors and methods for treating patients using the modified therapeutic cells.

### CROSS REFERENCE TO RELATED APPLICATIONS

This patent application claims priority to U.S. Provisional Application No. 63/263,637, filed Nov. 5, 2021, the disclosure of which is herein incorporated by reference in its entirety.

### STATEMENT REGARDING FEDERALLY SPONSORED RESEARCH OR DEVELOPMENT

This invention was made with government support under CA250401 awarded by National Institutes of Health. The government has certain rights in the invention.

### FIELD OF THE INVENTION

The application relates to chimeric cytokine receptors, particularly chimeric cytokine receptors comprising one or more leucine zipper motifs, and their uses in tumor immunotherapy (e.g., adoptive cell therapy). The application further relates to methods of genetically modifying therapeutic immune cells resulting in an enhanced immune response against a target antigen. The application further relates to therapeutic cells that express said chimeric cytokine receptors and methods for treating patients using the modified therapeutic cells.

### BACKGROUND

Despite recent advances in cancer treatment, patients with relapsed or refractory disease continue to have poor outcomes and novel approaches are needed. T cells that are genetically modified to express a chimeric antigen receptor (CAR) can kill chemotherapy-resistant tumor cells and therefore have the potential to improve outcomes and reduce treatment-related toxicity from conventional therapies [1, 2].

CARs typically consist of four components: i) an extracellular antigen recognition domain, most commonly a single chain variable fragment (scFv), ii) structural components, such as hinge and transmembrane domains, iii) a costimulatory domain that provides signals to sustain CAR T cell effector functions, and iv) a CD3(activation domain [1-3].

CAR T cells targeting CD19 have shown significant overall response rates against CD19-positive leukemia and lymphoma [4-6], leading to their FDA approval in 2017. In addition, CAR T cells targeting BCMA, CD30, CD22, or CD20 expressed on hematological malignancies have also shown significant activity in clinical studies [7-10]. However, a subset of patients does not achieve remission or relapses with antigen-positive disease due to suboptimal expansion or persistence of CAR T cells [11]. CAR T cells for the treatment of solid tumors are also actively being explored, but they have shown less impressive clinical results [12-16], most likely due to a multitude of factors that limit CAR T cell activity. Previous preclinical studies have demonstrated that improvements in CAR design, such as optimizing scFvs, modification of structural components, or modulation of CAR signaling can improve the antitumor activity of CAR T cells [17-21]. Likewise, additional genetic modification may be required to endow CAR T cells with potent and sustained effector function and to overcome the immunosuppressive tumor microenvironment (TME) to produce lasting benefits for cancer patients [22].

Physiological T cell activation requires three distinct signals for acquisition of effector function and formation of immunological memory. Signal 1 (activation) occurs via CD3ζ signal transduction following T cell receptor (TCR)-mediated antigen recognition. Signal 2 (costimulation) provides additional signals from CD28 or other molecules to augment signal 1. Finally, signal 3, mediated by cytokines, is required for optimal T cell proliferation, differentiation of naïve T cells into effector cells, and development of functional T cell memory [23-25].

First generation CARs provide only signal 1, via CD3ζ. Second-generation CARs also provide signal 2, most often through CD28 or 4-1BB co-stimulation to sustain CAR T cell expansion following activation. Although activated CAR T cells produce cytokines, such as interleukin-2 (IL-2), production decreases after repeated exposure to tumor cells [26], and some cytokines that are important for T cell effector function, such as IL-12 and IL-15, are either produced at low levels are not at all by T cells [27,28]. Due to these limitations, there is a need to engineer CAR T cells to augment cytokine-mediated signals.

### SUMMARY OF THE INVENTION

Disclosed herein are chimeric cytokine receptors, particularly chimeric cytokine receptors comprising one or more leucine zipper motifs, and their uses in tumor immunotherapy (e.g., adoptive cell therapy). Additionally disclosed are methods of genetically modifying therapeutic immune cells resulting in an enhanced immune response against a target antigen. Therapeutic cells that express said chimeric cytokine receptors and methods for treating patients using the modified therapeutic cells are also disclosed.

In one aspect is provided a chimeric cytokine receptor comprising
a) a first polypeptide comprising:
   i) a first extracellular region comprising one or more leucine zipper motifs,
   ii) optionally, a first hinge region,
   iii) a first transmembrane region, and
   iv) at least a first intracellular signaling region derived from a first cytokine receptor chain; and
b) a second polypeptide comprising:
   i) a second extracellular region comprising one or more leucine zipper motifs,
   ii) optionally, a second hinge region,
   iii) a second transmembrane region, and
   iv) at least a second intracellular signaling region derived from a second cytokine receptor chain,
      wherein the one or more leucine zipper motifs from the first extracellular region heterodimerize with the one or more leucine zipper motifs from the second extracellular region.

In some embodiments, each extracellular region may comprise two leucine zipper motifs.

In some embodiments, each leucine zipper motif of the first or second extracellular region may comprise at least five heptad repeats of amino acids with a leucine at every seventh position.

In some embodiments of any of the chimeric cytokine receptors disclosed herein, a) each leucine zipper motif of the first extracellular region may comprise the amino acid sequence of LEIEAAFLERENTALETRVAELRQRVQRLRNRVSQYRTRYGPL (SEQ ID NO: 1), and each leucine zipper motif of the second extracellular region comprises the amino acid sequence of LEIRAAFLRQRNTALRTEVAELEQEVQRLENEVSQYETRYGPL (SEQ ID NO: 2); or b) each leucine zipper motif of the first extracellular region may comprise the amino acid sequence of LEIRAAFLRQRNTALRTEVAELEQEVQRLENEVSQYETRYGPL (SEQ ID NO: 2), and each leucine zipper motif of the second extracellular region may comprise the amino acid sequence of LEIEAAFLERENTALETRVAELRQRVQRLRNRVSQYRTRYGPL (SEQ ID NO: 1).

In some embodiments, the at least two leucine zipper motifs may be operatively linked to each other via a linker.

In some embodiments, the linker may comprise the amino acid sequence of GGGGSGGGGSGGGGS (SEQ ID NO: 11).

In some embodiments, the at least one first and/or second intracellular signaling regions may be derived from cytokine receptor chain(s) of a type I cytokine receptor.

In some embodiments, the at least one first and/or second intracellular signaling regions may be derived from cytokine receptor chain(s) of a type II cytokine receptor.

In some embodiments, the at least one first and/or second intracellular signaling regions may be derived from cytokine receptor chain(s) of the IL-2 receptor, IL-7 receptor, IL-15 receptor, IL-21 receptor, IL-12 receptor, IL-9 receptor, TL-4 receptor, IL-23 receptor, IL-10 receptor, or IL-27 receptor, or a combination thereof.

In some embodiments, the at least one first and/or second intracellular signaling regions may be derived from cytokine receptor chain(s) of a gamma cytokine receptor.

In some embodiments, the first or the second intracellular signaling region may be derived from the common gamma chain (γc) (also known as TL-2 receptor gamma chain or IL-2RG, or CD132).

In some embodiments, the first intracellular signaling region may be derived from the common gamma chain (γc), and the second intracellular signaling region may be derived from the TL-2 receptor beta chain.

In some embodiments, the first intracellular signaling region may be derived from the common gamma chain (γc), and the second intracellular signaling region may be derived from the TL-7 receptor alpha chain.

In some embodiments, the first intracellular signaling region may be derived from the common gamma chain (γc), and the second intracellular signaling region may be derived from the IL-21 receptor chain.

In some embodiments, the first intracellular signaling region may be derived from the common gamma chain (γc), and the second intracellular signaling region may be derived from the TL-9 receptor chain.

In some embodiments, the first intracellular signaling region may be derived from the common gamma chain (γc), and the second intracellular signaling region may be derived from the TL-4 receptor alpha chain.

In some embodiments, the first intracellular signaling region may be derived from the IL-12 receptor beta 1 chain, and the second intracellular signaling region may be derived from the IL-12 receptor beta 2 chain.

In some embodiments, the first intracellular signaling region may be derived from the IL-23 receptor chain, and the second intracellular signaling region may be derived from the IL-12 receptor beta 2 chain.

In some embodiments, the first intracellular signaling region may be derived from the IL-10 receptor alpha chain, and the second intracellular signaling region may be derived from the IL-10 receptor beta chain.

In some embodiments, the first intracellular signaling region may be derived from the IL-27 receptor alpha chain (or WSX-1), and the second intracellular signaling region may be derived from glycoprotein 130 (gp130 or IL-6 beta chain).

In some embodiments, the first and/or the second polypeptides may comprise two intracellular signaling regions, which may be derived from two cytokine receptor chains.

In some embodiments, the first polypeptide may comprise an intracellular signaling region derived from the IL-2 receptor beta chain and an intracellular signaling region derived from the IL-21 receptor chain, and the second polypeptide may comprise an intracellular signaling region derived from the common gamma chain (γc).

In some embodiments, the first polypeptide may comprise an intracellular signaling region derived from the IL-7 receptor alpha chain and an intracellular signaling region derived from the IL-21 receptor chain, and the second polypeptide may comprise an intracellular signaling region derived from the common gamma chain (γc).

In some embodiments, the first hinge region may be derived from the same first cytokine receptor chain as the first intracellular signaling region.

In some embodiments, the second hinge region may be derived from the same second cytokine receptor chain as the second intracellular signaling region.

In some embodiments, the first and/or second hinge regions may be derived from cytokine receptor chain(s) of the IL-2 receptor, IL-7 receptor, IL-15 receptor, IL-21 receptor, IL-12 receptor, IL-9 receptor, IL-4 receptor, IL-23 receptor, IL-10 receptor, or IL-27 receptor.

In some embodiments, the first and/or second hinge regions may be derived from cytokine receptor chain(s) of a gamma cytokine receptor.

In some embodiments, the first or the second hinge region may be derived from the common gamma chain (γc).

In some embodiments, the first and/or second hinge regions may be derived from a molecule different from the first and/or second cytokine receptor chain(s) from which the first and/or second intracellular signaling region(s) may be derived.

In some embodiments, the first and/or second hinge regions may be derived from IgG1, IgG2, IgG3, IgG4, CD28, or CD8a.

In some embodiments, the first and second hinge regions may be the same.

In some embodiments, the first and second hinge regions may be different.

In some embodiments, the first transmembrane region may be derived from the same cytokine receptor chain as the first intracellular signaling region.

In some embodiments, the second transmembrane region may be derived from the same cytokine receptor chain as the second intracellular signaling region.

In some embodiments, the first and/or second transmembrane regions may be derived from cytokine receptor chain(s) of the IL-2 receptor, IL-7 receptor, IL-15 receptor, IL-21 receptor, IL-12 receptor, IL-9 receptor, IL-4 receptor, IL-23 receptor, IL-10 receptor, or IL-27 receptor.

In some embodiments, the first and/or second transmembrane regions may be derived from a gamma cytokine receptor.

In some embodiments, the first or the second transmembrane region may be derived from a common gamma chain (γc).

In some embodiments, the first and/or second transmembrane regions may be derived from a molecule different from the first and/or second cytokine receptor chain(s) from which the first and/or second intracellular signaling region(s) may be derived.

In some embodiments, the first and/or second transmembrane regions may be derived from CD28, CD8, CD4, CD3ζ, CD40, CD134 (OX-40), CD19, or CD7.

In some embodiments, the first and second transmembrane regions may be the same.

In some embodiments, the first and second transmembrane regions may be different.

In some aspects of the chimeric cytokine receptor disclosed herein, the chimeric cytokine receptor may comprise
a) a first polypeptide comprising:
   i) a first extracellular region comprising one or more leucine zipper motifs,
   ii) optionally, a first hinge region derived from the common gamma chain (γc),
   iii) a first transmembrane region derived from the common gamma chain (γc), and
   iv) a first intracellular signaling region derived from the common gamma chain (γc); and
b) a second polypeptide comprising:
   i) a second extracellular region comprising one or more leucine zipper motifs,
   ii) optionally, a second hinge region derived from the IL-2 receptor beta chain,
   iii) a second transmembrane region derived from the IL-2 receptor beta chain, and
   iv) a second intracellular signaling region derived from the IL-2 receptor beta chain.

In some aspects of the chimeric cytokine receptor disclosed herein, the chimeric cytokine receptor may comprise
a) a first polypeptide comprising:
   i) a first extracellular region comprising at least one or more leucine zipper motifs,
   ii) optionally, a first hinge region derived from the common gamma chain (γc),
   iii) a first transmembrane region derived from the common gamma chain (γc), and
   iv) a first intracellular signaling region derived from the common gamma chain (γc); and
b) a second polypeptide comprising:
   i) a second extracellular region comprising one or more leucine zipper motifs,
   ii) optionally, a second hinge region derived from the IL-7 receptor alpha chain,
   iii) a second transmembrane region derived from the IL-7 receptor alpha chain, and
   iv) a second intracellular signaling region derived from the IL-7 receptor alpha chain.

In some aspects of the chimeric cytokine receptor disclosed herein, the chimeric cytokine receptor may comprise
a) a first polypeptide comprising:
   i) a first extracellular region comprising one or more leucine zipper motifs,
   ii) optionally, a first hinge region derived from the common gamma chain (γc),
   iii) a first transmembrane region derived from the common gamma chain (γc), and
   iv) a first intracellular signaling region derived from the common gamma chain (γc); and
b) a second polypeptide comprising:
   i) a second extracellular region comprising one or more leucine zipper motifs,
   ii) optionally, a second hinge region derived from the IL-21 receptor chain,
   iii) a second transmembrane region derived from the IL-21 receptor chain, and
   iv) a second intracellular signaling region derived from the IL-21 receptor chain.

In some aspects of the chimeric cytokine receptor disclosed herein, the chimeric cytokine receptor may comprise
a) a first polypeptide comprising:
   i) a first extracellular region comprising one or more leucine zipper motifs,
   ii) optionally, a first hinge region derived from the common gamma chain (γc),
   iii) a first transmembrane region derived from the common gamma chain (γc), and
   iv) a first intracellular signaling region derived from the common gamma chain (γc); and
b) a second polypeptide comprising:
   i) a second extracellular region comprising one or more leucine zipper motifs,
   ii) optionally, a second hinge region derived from the IL-9 receptor chain,
   iii) a second transmembrane region derived from the IL-9 receptor chain, and
   iv) a second intracellular signaling region derived from the IL-9 receptor chain.

In some aspects of the chimeric cytokine receptor disclosed herein, the chimeric cytokine receptor may comprise
a) a first polypeptide comprising:
   i) a first extracellular region comprising one or more leucine zipper motifs,
   ii) optionally, a first hinge region derived from the common gamma chain (γc),
   iii) a first transmembrane region derived from the common gamma chain (γc), and
   iv) a first intracellular signaling region derived from the common gamma chain (γc); and
b) a second polypeptide comprising:
   i) a second extracellular region comprising one or more leucine zipper motifs,
   ii) optionally, a second hinge region derived from the IL-4 receptor alpha chain,
   iii) a second transmembrane region derived from the IL-4 receptor alpha chain, and
   iv) a second intracellular signaling region derived from the IL-4 receptor alpha chain.

In some aspects of the chimeric cytokine receptor disclosed herein, the chimeric cytokine receptor may comprise
a) a first polypeptide comprising:
   i) a first extracellular region comprising one or more leucine zipper motifs,
   ii) optionally, a first hinge region derived from the IL-12 receptor beta 1 chain,
   iii) a first transmembrane region derived from the IL-12 receptor beta 1 chain, and
   iv) a first intracellular signaling region derived from the IL-12 receptor beta 1 chain; and
b) a second polypeptide comprising:
   i) a second extracellular region comprising one or more leucine zipper motifs,
   ii) optionally, a second hinge region derived from the IL-12 receptor beta 2 chain,
   iii) a second transmembrane region derived from the IL-12 receptor beta 2 chain, and
   iv) a second intracellular signaling region derived from the IL-12 receptor beta 2 chain.

In some aspects of the chimeric cytokine receptor disclosed herein, the chimeric cytokine receptor may comprise
a) a first polypeptide comprising:
   i) a first extracellular region comprising one or more leucine zipper motifs,
   ii) optionally, a first hinge region derived from the IL-23 receptor chain,
   iii) a first transmembrane region derived from the IL-23 receptor chain, and
   iv) a first intracellular signaling region derived from the IL-23 receptor chain; and
b) a second polypeptide comprising:
   i) a second extracellular region comprising one or more leucine zipper motifs,
   ii) optionally, a second hinge region derived from the IL-12 receptor beta 2 chain,
   iii) a second transmembrane region derived from the IL-12 receptor beta 2 chain, and
   iv) a second intracellular signaling region derived from the IL-12 receptor beta 2 chain.

In some aspects of the chimeric cytokine receptor disclosed herein, the chimeric cytokine receptor may comprise
a) a first polypeptide comprising:
   i) a first extracellular region comprising one or more leucine zipper motifs,
   ii) optionally, a first hinge region derived from the IL-10 receptor alpha chain,
   iii) a first transmembrane region derived from the IL-10 receptor alpha chain, and
   iv) a first intracellular signaling region derived from the IL-10 receptor alpha chain; and
b) a second polypeptide comprising:
   i) a second extracellular region comprising one or more leucine zipper motifs,
   ii) optionally, a second hinge region derived from the IL-10 receptor beta chain,
   iii) a second transmembrane region derived from the IL-10 receptor beta chain, and
   iv) a second intracellular signaling region derived from the IL-10 receptor beta chain.

In some aspects of the chimeric cytokine receptor disclosed herein, the chimeric cytokine receptor may comprise
a) a first polypeptide comprising:
   i) a first extracellular region comprising one or more leucine zipper motifs,
   ii) optionally, a first hinge region derived from the IL-27 receptor alpha chain (or WSX-1),
   iii) a first transmembrane region derived from the IL-27 receptor alpha chain (or WSX-1), and
   iv) a first intracellular signaling region derived from the IL-27 receptor alpha chain (or WSX-1); and
b) a second polypeptide comprising:
   i) a second extracellular region comprising one or more leucine zipper motifs,
   ii) optionally, a second hinge region derived from glycoprotein 130 (gp130),
   iii) a second transmembrane region derived from glycoprotein 130 (gp130), and
   iv) a second intracellular signaling region derived from glycoprotein 130 (gp130).

In some aspects of the chimeric cytokine receptor disclosed herein, the chimeric cytokine receptor may comprise
a) a first polypeptide comprising:
   i) a first extracellular region comprising one or more leucine zipper motifs,
   ii) optionally, a first hinge region derived from the common gamma chain (γc),
   iii) a first transmembrane region derived from the common gamma chain (γc), and
   iv) a first intracellular signaling region derived from the common gamma chain (γc); and
b) a second polypeptide comprising:
   i) a second extracellular region comprising one or more leucine zipper motifs,
   ii) optionally, a second hinge region derived from IL-2 receptor beta chain,
   iii) a second transmembrane region derived from IL-2 receptor beta chain, and
   iv) a second intracellular signaling region derived from IL-2 receptor beta chain; and
   v) a third intracellular signaling region derived from the IL-21 receptor chain.

In some embodiments, the intracellular signaling region may be derived from the common gamma chain (γc) comprises the amino acid sequence of SEQ ID NO: 34, or an amino acid sequence having at least 80% identity thereof.

In some embodiments, the hinge region may be derived from the common gamma chain (7c) and may comprise the amino acid sequence of SEQ ID NO: 30, or a sequence having at least 80% identity thereof.

In some embodiments, the transmembrane region may be derived from the common gamma chain (7c) and may comprise the amino acid sequence of SEQ ID NO: 32, or a sequence having at least 80% identity thereof.

In some embodiments, the intracellular signaling region may be derived from the IL-2 receptor beta chain and may comprise the amino acid sequence of SEQ ID NO: 18, or an amino acid sequence having at least 80% identity thereof.

In some embodiments, the hinge region may be derived from the IL-2 receptor beta chain and may comprise the amino acid sequence of SEQ ID NO: 14, or a sequence having at least 80% identity thereof.

In some embodiments, the transmembrane region may be derived from the IL-2 receptor beta chain and may comprise the amino acid sequence of SEQ ID NO: 16, or a sequence having at least 80% identity thereof.

In some embodiments, the intracellular signaling region may be derived from the IL-7 receptor alpha chain and may comprise the amino acid sequence of SEQ ID NO: 48, or an amino acid sequence having at least 80% identity thereof.

In some embodiments, the hinge region may be derived from the IL-7 receptor alpha chain and may comprise the amino acid sequence of SEQ ID NO: 44, or a sequence having at least 80% identity thereof.

In some embodiments, the transmembrane region may be derived from the IL-7 receptor alpha chain and may comprise the amino acid sequence of SEQ ID NO: 46, or a sequence having at least 80% identity thereof.

In some embodiments, the intracellular signaling region may be derived from the IL-21 receptor chain and may comprise the amino acid sequence of SEQ ID NO: 58, or an amino acid sequence having at least 80% identity thereof.

In some embodiments, the hinge region may be derived from the IL-21 receptor chain and may comprise the amino acid sequence of SEQ ID NO: 54, or a sequence having at least 80% identity thereof.

In some embodiments, the transmembrane region may be derived from the IL-21 receptor chain and may comprise the amino acid sequence of SEQ ID NO: 56, or a sequence having at least 80% identity thereof.

In some embodiments, the intracellular signaling region may be derived from the IL-9 receptor chain and may comprise the amino acid sequence of SEQ ID NO: 68, or an amino acid sequence having at least 80% identity thereof.

In some embodiments, the hinge region may be derived from the IL-9 receptor chain and may comprise the amino acid sequence of SEQ ID NO: 64, or a sequence having at least 80% identity thereof.

In some embodiments, the transmembrane region may be derived from the IL-9 receptor chain and may comprise the amino acid sequence of SEQ ID NO: 66, or a sequence having at least 80% identity thereof.

In some embodiments, the intracellular signaling region may be derived from the IL-4 receptor alpha chain and may comprise the amino acid sequence of SEQ ID NO: 78, or an amino acid sequence having at least 80% identity thereof.

In some embodiments, the hinge region may be derived from the IL-4 receptor alpha chain and may comprise the amino acid sequence of SEQ ID NO: 74, or a sequence having at least 80% identity thereof.

In some embodiments, the transmembrane region may be derived from the IL-4 receptor alpha chain and may comprise the amino acid sequence of SEQ ID NO: 76, or a sequence having at least 80% identity thereof.

In some embodiments, the intracellular signaling region may be derived from the IL-12 receptor beta 1 chain and may comprise the amino acid sequence of SEQ ID NO: 88, or an amino acid sequence having at least 80% identity thereof.

In some embodiments, the hinge region may be derived from the IL-12 receptor beta 1 chain and may comprise the amino acid sequence of SEQ ID NO: 84, or a sequence having at least 80% identity thereof.

In some embodiments, the transmembrane region may be derived from the IL-12 receptor beta 1 chain and may comprise the amino acid sequence of SEQ ID NO: 86, or a sequence having at least 80% identity thereof.

In some embodiments, the intracellular signaling region may be derived from the IL-12 receptor beta 2 chain and may comprise the amino acid sequence of SEQ ID NO: 98, or an amino acid sequence having at least 80% identity thereof.

In some embodiments, the hinge region may be derived from the IL-12 receptor beta 2 chain and may comprises the amino acid sequence of SEQ ID NO: 94, or a sequence having at least 80% identity thereof.

In some embodiments, the transmembrane region may be derived from the IL-12 receptor beta 2 chain and may comprise the amino acid sequence of SEQ ID NO: 96, or a sequence having at least 80% identity thereof.

In some embodiments, the intracellular signaling region may be derived from the IL-23 receptor chain and may comprise the amino acid sequence of SEQ ID NO: 108, or an amino acid sequence having at least 80% identity thereof.

In some embodiments, the hinge region may be derived from the IL-23 receptor chain and may comprise the amino acid sequence of SEQ ID NO: 104, or a sequence having at least 80% identity thereof.

In some embodiments, the transmembrane region may be derived from the IL-23 receptor chain and may comprise the amino acid sequence of SEQ ID NO: 106, or a sequence having at least 80% identity thereof.

In some embodiments, the intracellular signaling region may be derived from the IL-10 receptor alpha chain and may comprise the amino acid sequence of SEQ ID NO: 118, or an amino acid sequence having at least 80% identity thereof.

In some embodiments, the hinge region may be derived from the IL-10 receptor alpha chain and may comprise the amino acid sequence of SEQ ID NO: 114, or a sequence having at least 80% identity thereof.

In some embodiments, the transmembrane region may be derived from the IL-10 receptor alpha chain and may comprise the amino acid sequence of SEQ ID NO: 116, or a sequence having at least 80% identity thereof.

In some embodiments, the intracellular signaling region may be derived from the IL-10 receptor beta chain and may comprise the amino acid sequence of SEQ ID NO: 128, or an amino acid sequence having at least 80% identity thereof.

In some embodiments, the hinge region may be derived from the IL-10 receptor beta chain and may comprise the amino acid sequence of SEQ ID NO: 124, or a sequence having at least 80% identity thereof.

In some embodiments, the transmembrane region may be derived from the IL-10 receptor beta chain and may comprise the amino acid sequence of SEQ ID NO: 126, or a sequence having at least 80% identity thereof.

In some embodiments, the intracellular signaling region may be derived from gp130 and may comprise the amino acid sequence of SEQ ID NO: 138, or an amino acid sequence having at least 80% identity thereof.

In some embodiments, the hinge region may be derived from gp130 and may comprise the amino acid sequence of SEQ ID NO: 134, or a sequence having at least 80% identity thereof.

In some embodiments, the transmembrane region may be derived from gp130 and may comprise the amino acid sequence of SEQ ID NO: 136, or a sequence having at least 80% identity thereof.

In some embodiments, the first polypeptide may comprise the amino acid sequence of SEQ ID NO: 42, or a sequence having at least 80% identity thereof, and/or the second polypeptide may comprise the amino acid sequence of SEQ ID NO: 40, or a sequence having at least 80% identity thereof.

In some embodiments, the first polypeptide may comprise the amino acid sequence of SEQ ID NO: 42, or a sequence having at least 80% identity thereof, and/or the second polypeptide may comprise the amino acid sequence of SEQ ID NO: 52, or a sequence having at least 80% identity thereof.

In some embodiments, the first polypeptide may comprise the amino acid sequence of SEQ ID NO: 42, or a sequence having at least 80% identity thereof, and/or the second polypeptide may comprise the amino acid sequence of SEQ ID NO: 62, or a sequence having at least 80% identity thereof.

In some embodiments, the first polypeptide may comprise the amino acid sequence of SEQ ID NO: 42, or a sequence having at least 80% identity thereof, and/or the second polypeptide may comprise the amino acid sequence of SEQ ID NO: 72, or a sequence having at least 80% identity thereof.

In some embodiments, the first polypeptide may comprise the amino acid sequence of SEQ ID NO: 42, or a sequence having at least 80% identity thereof, and/or the second polypeptide may comprise the amino acid sequence of SEQ ID NO: 82, or a sequence having at least 80% identity thereof.

In some embodiments, the first polypeptide may comprise the amino acid sequence of SEQ ID NO: 92, or a sequence having at least 80% identity thereof, and/or the second polypeptide may comprise the amino acid sequence of SEQ ID NO: 102, or a sequence having at least 80% identity thereof.

In some embodiments, the first polypeptide may comprise the amino acid sequence of SEQ ID NO: 112, or a sequence having at least 80% identity thereof, and/or the second polypeptide may comprise the amino acid sequence of SEQ ID NO: 102, or a sequence having at least 80% identity thereof.

In some embodiments, the first polypeptide may comprise the amino acid sequence of SEQ ID NO: 122, or a sequence having at least 80% identity thereof, and/or the second polypeptide may comprise the amino acid sequence of SEQ ID NO: 132, or a sequence having at least 80% identity thereof.

In some embodiments, the first polypeptide may comprise the amino acid sequence of SEQ ID NO: 142, or a sequence having at least 80% identity thereof, and/or the second polypeptide may comprise the amino acid sequence of SEQ ID NO: 152, or a sequence having at least 80% identity thereof.

In some embodiments, the first and/or the second polypeptide may further comprise a leader sequence.

In some embodiments, the leader sequence may be derived from an immunoglobulin heavy chain variable region or colony stimulating factor 2 receptor alpha chain (CSF2RA).

In some embodiments, the leader sequence may be derived from an immunoglobulin heavy chain variable region and may comprise the amino acid sequence MDWIWRILFLVGAATGAHS (SEQ ID NO: 3).

In some embodiments, the first and second leader sequences may be the same.

In some embodiments, the first and second leader sequences may be different.

In some embodiments, the first and/or second polypeptide may further comprise one or more additional polypeptide sequences.

In some embodiments, the one or more additional polypeptide sequences may be selected from one or more cellular markers, epitope tags, cytokines, safety switches, dimerization moieties, or degradation moieties.

In another aspect is provided a polynucleotide encoding any of the chimeric cytokine receptors disclosed herein.

In some embodiments, the polynucleotide may comprise
a) a nucleotide sequence encoding the first polypeptide of the chimeric cytokine receptor; and
b) a nucleotide sequence encoding the second polypeptide of the chimeric cytokine receptor.

In some embodiments, the nucleotide sequence encoding the first polypeptide of the chimeric cytokine receptor may be operably linked to the first polypeptide of the chimeric cytokine receptor via a sequence encoding a self-cleaving peptide and/or an internal ribosomal entry site (IRES).

In some embodiments, the self-cleaving peptide may be a 2A peptide.

In some embodiments, the 2A peptide may be T2A, P2A, E2A, or F2A peptide.

In some embodiments, the 2A peptide may be a P2A peptide.

In some embodiments, the P2A peptide may comprise the amino acid sequence GSGATNFSLLKQAGDVEENPGP (SEQ ID NO: 22), or an amino acid sequence having at least 80% sequence identity thereof.

In some aspects is provided a polynucleotide comprising a nucleotide sequence encoding the first polypeptide of any of the chimeric cytokine receptors disclosed herein.

In some aspects is provided a polynucleotide comprising a nucleotide sequence encoding the second polypeptide of any of the chimeric cytokine receptors

In some embodiments, the nucleotide sequence(s) may be expressed in an inducible fashion, achieved with an inducible promoter, an inducible expression system, an artificial signaling circuits, and/or drug induced splicing.

In some embodiments, the nucleotide sequences encoding the first and second polypeptides of the chimeric cytokine receptor may be operably linked to a single promoter.

In some embodiments, the nucleotide sequence encoding the first polypeptide of the chimeric cytokine receptor may be operably linked to a first promoter.

In some embodiments, the nucleotide sequence encoding the second polypeptide of the chimeric cytokine receptor may be operably linked to a second promoter.

In some embodiments, the nucleotide sequence encoding the first polypeptide of the chimeric cytokine receptor may be operably linked to a first promoter, the nucleotide sequence encoding the second polypeptide of the chimeric cytokine receptor may be operably linked to a second promoter, and the first and second promoters may be the same.

In some embodiments, the nucleotide sequence encoding the first polypeptide of the chimeric cytokine receptor may be operably linked to a first promoter, the nucleotide sequence encoding the second polypeptide of the chimeric cytokine receptor may be operably linked to a second promoter, and the first and second promoters may be different.

In some embodiments, the promoter may be an inducible promoter.

In some embodiments, the promoter may be a T cell-specific promoter or an NK cell-specific promoter.

In some embodiments, any of the polynucleotides disclosed herein may comprise one or more additional nucleotide sequences encoding one or more additional polypeptide sequences.

In some embodiments, the one or more additional polypeptide sequences may be selected from one or more cellular markers, epitope tags, cytokines, safety switches, dimerization moieties, or degradation moieties.

In some embodiments, the epitope tag may be FLAG or Myc.

In some embodiments, the cellular marker may be mClover3 or mRuby.

In some embodiments, any of the polynucleotides disclosed herein may be a DNA molecule.

In some embodiments, any of the polynucleotides disclosed herein may be an RNA molecule.

In another aspect is provided a recombinant vector comprising any of the polynucleotides disclosed herein.

In some embodiments, the vector may be a viral vector.

In some embodiments, the viral vector may be a retroviral vector, a lentiviral vector, an adenoviral vector, an adeno-associated virus vector, an alphaviral vector, a herpes virus vector, a baculoviral vector, or a vaccinia virus vector.

In some embodiments, the viral vector may be a retroviral vector.

In some embodiments, the vector may be a non-viral vector.

In some embodiments, the non-viral vector may be a minicircle plasmid, a Sleeping Beauty transposon, a piggyBac transposon, or a single or double stranded DNA molecule that may be used as a template for homology directed repair (HDR) based gene editing.

In another aspect is provided an isolated host cell comprising any of the polynucleotides disclosed herein or any of the recombinant vectors disclosed herein.

In another aspect is provided an isolated host cell comprising any of the chimeric cytokine receptors encoded by any of the polynucleotide disclosed herein.

In some embodiments, the host cell may be an immune cell.

In some embodiments, the host cell may be a T cell, a natural killer (NK) cell, a mesenchymal stem cell (MSC), or a macrophage.

In some embodiments, the host cell may be a T cell.

In some embodiments, the host cell may be an αβ T-cell receptor (TCR) T-cell, a γδ T-cell, a CD8+ T-cell, a CD4+ T-cell, a cytotoxic T-cell, an invariant natural killer T (iNKT) cell, a memory T-cell, a memory stem T-cell (TSCM), a naïve T-cell, an effector T-cell, a T-helper cell, or a regulatory T-cell (Treg).

In some embodiments, the host cell may be a NK cell derived from peripheral, cord blood, iPSCs, and/or a cell line (e.g., NK-92 cells).

In some embodiments, the host cell may further express one or more antigen-recognition molecules.

In some embodiments, the one or more antigen-recognition molecules may be selected from αβ T cell receptors (TCRs), synthetic T cell receptors and antigen receptor (STARs), chimeric antigen receptor (CARs), T cell antigen couplers (TACs), T cell receptor fusion constructs (TruCs), or antibodies (e.g., bispecific antibodies), or a combination thereof.

In some embodiments, the host cell may be further genetically modified to enhance its function, for example, by expressing one or more additional genes (e.g., transcription factors (c-Jun)) or deleting one or more inhibitory genes (e.g., REGNASE-1, DNMT3A) with gene editing technologies (e.g., CRISPR-Cas9 or transcription activator-like effector nucleases (TALENs)).

In some embodiments, the host cell may be activated and/or expanded ex vivo.

In some embodiments, the host cell may be an allogeneic cell.

In some embodiments, the host cell may be an autologous cell.

In some embodiments, the immune cells disclosed herein may be derived from an induced pluripotent stem (iPS) cells.

In another aspect is provided a pharmaceutical composition comprising any of the host cell disclosed herein and a pharmaceutically acceptable carrier and/or excipient.

In another aspect is provided a method of enhancing effector function of an immune cell, wherein the immune cell may express a chimeric antigen receptor (CAR), comprising genetically modifying the cell with a polynucleotide or a recombinant vector of the present disclosure.

In some embodiments, the effector function may be one or more of expansion, persistence, and/or anti-tumor activity.

In another aspect is provided a method of generating the isolated host cell disclosed herein, said method comprising genetically modifying the host cell with a polynucleotide or a recombinant vector of the present disclosure.

In some embodiments, methods disclosed herein may further comprising genetically modifying a host cell of the present disclosure to express a chimeric antigen receptor (CAR).

In some embodiments, the genetic modifying step may be conducted via viral gene delivery.

In some embodiments, the genetic modifying step may be conducted via non-viral gene delivery.

In some embodiments, the genetically modifying step may be conducted ex vivo.

In some embodiments, the method disclosed herein may further comprise activation and/or expansion of the host cell ex vivo before, after and/or during the genetic modification.

In some embodiments, the host cell may be an immune cell.

In some embodiments, the immune cell may be a T cell, a natural killer (NK) cell, a mesenchymal stem cell (MSC), or a macrophage.

In some embodiments, the cell may be a T cell.

In some embodiments, the cell may be an αβ T-cell receptor (TCR) T-cell, a γδ T-cell, a CD8+ T-cell, a CD4+ T-cell, a cytotoxic T-cell, an invariant natural killer T (iNKT) cell, a memory T-cell, a memory stem T-cell (TSCM), a naïve T-cell, an effector T-cell, a T-helper cell, or a regulatory T-cell (Treg).

In some embodiments, the cell may be a NK cell, which may be derived from peripheral, cord blood, iPSCs, and/or a cell line (e.g., NK-92 cell).

In another aspect is provided a method of treating a disease comprising administering to the subject an effective amount of any of the host cells disclosed herein, or a pharmaceutical composition of the present disclosure.

In some embodiments, the method may comprise
a) isolating T cells or NK cells from the subject or donor;
b) modifying said T cells or NK cells ex vivo with a polynucleotide disclosed herein or a recombinant vector disclosed herein;
c) optionally modifying said T cells or NK cells ex vivo to express a chimeric antigen receptor (CAR) that binds an antigen associated with said disease;
d) optionally, expanding and/or activating the modified T cells or NK cells before, after and/or during step b) or c); and
e) introducing a therapeutically effective amount of the modified T cells or NK cells into the subject.

In some embodiments, the disease is a cancer, infection, or autoimmune disease.

In some embodiments, the subject is human.

### BRIEF DESCRIPTION OF THE DRAWINGS

FIGS. 1A-IC display schematic representations of exemplary “Zip Cytokine Receptor” (zipReceptor) designs. (FIG. 1A) Schematic representation of an exemplary zipReceptor general design, and of exemplary zip IL-2 Receptor (zipIL-2R), zip IL-7 Receptor (zipIL-7R), and zip IL-21 Receptor (zipIL-21R) designs. (FIG. 1B) Schematic representation of exemplary zipReceptor designs comprising other receptors, for example, those comprising natural dimeric chains, including zipIL-9R, zipIL-4R, zipIL-12R, zipIL-23R, zipIL-10R, and zipIL-27R. (FIG. 1C). Schematic representation of any number of various additional exemplary zipReceptor designs, which may comprise other combinations of, e.g., extracellular (EC), transmembrane (TM), and/or intracellular (IC) domains, and which may form receptors that may not exist in nature. A schematic representation of a zipIL-2/IL-21 receptor design is a non-limiting example of a zipReceptor comprising engineered receptor(s).

FIGS. 2A-2E show that the 2X leucine zipper IL-2 Receptor (zipIL-2R) activates STAT5 and improves T cell survival. (FIG. 2A) Schematic representation of 1X and 2X leucine zipper motifs to activate engineered IL-2 receptors (IL-2Rs). (FIG. 2B) Representative flow cytometry plots of human T cells transduced with functional zipIL-2R with 1X or 2X leucine zipper motifs. IL-2Rβ.1X chain detected by mRuby expression, IL-2Rγ.1X chain detected by mClover expression, and zipIL-2R detected by mClover expression. NT: non-transduced. (FIG. 2C) Quantification of transduction efficiency and phosphorylated STAT5 (pSTAT5) expression in human T cells transduced with zipIL-2R constructs. (FIG. 2D) Representative flow cytometry of transduced cells stained with Annexin V and a dead cell marker. (FIG. 2E) Quantification of dead cells (Annexin V+ Dead+), early apoptotic cells (Annexin V+), necrotic cells (Dead+), and live cells (Annexin V− Dead−) in transduced human T cells after seven days of cytokine starvation. N=3 biological replicates. Error bars indicate standard deviation (SD). **p<0.01, ***p<0.001, ****p<0.0001 according to 2-way ANOVA with Tukey's multiple comparison test.

FIGS. 3A-3D show two leucine zipper domains enable oligomerization of zipReceptors. (FIGS. 3A-3C) Structural modeling of zipReceptor components predicted by AlphaFold. (FIG. 3A) Single zipper viewed from front (top) and top (bottom). (FIG. 3B) Two different conformations for the dual zipper binding in parallel or antiparallel orientations. (FIG. 3C) The transmembrane (TM) and extracellular (EC) domain of single (left) and dual (right) zippers. The starting residue of leucine zipper domains, methionine, is shown as a green sphere. Black lines denote the membrane location. (FIG. 3D) Free-hand drawing of predicted higher-order oligomerization of 2X zipReceptors via the domain swapping model.

FIGS. 4A-4J show zipIL-2R enhances B7-H3-specific CAR T cell antitumor activity in vitro without altering antigen specificity. (FIG. 4A) Transduction of human T cells with functional B7-H3-specific CAR (CAR), non-functional B7-H3-specific CAR (Δ CAR) and/or zipIL-2R; n=4-6. (FIG. 4B) Immuno-phenotype of human T cells transduced with CAR and/or zipTL-2R constructs. T_{N}-Like: CD45RA+CCR7+, T_{CM}: CD45RA− CCR7+, T_{EM}: CD45RA− CCR7−, T_{EMRA}: CD45RA+ CCR7−; n=4. (FIG. 4C) 24 hour MTS cytotoxicity assay with B7-H3 knockout (KO) A549 or LM7 tumor cells co-cultured with CAR, CAR+zipTL-2R, ΔCAR, or ΔCAR+zipTL-2R T cells at different effector cell to target cell ratios (E:T); n=4. (FIG. 4D) 24 hour MTS cytotoxicity assay with B7-H3+(Wild-Type, WT) A549 or LM7 tumor cells co-cultured with CAR, CAR+zipIL-2R, ΔCAR, or ΔCAR+zipIL-2R T cells at different effector cell to target cell ratios (E:T); n=4. (FIGS. 4E-4J) Expansion of CAR, CAR+zipIL-2R, ΔCAR, or ΔCAR+zipIL-2R T cells after weekly serial co-culture with indicated tumor cell lines at a 2:1 E:T ratio. Quantification of relative expansion of CAR+zipIL-2R T cells compared to CAR T cells at different rounds of stimulation when co-cultured with A549 or LM7 cells (FIG. 4G and FIG. 4J). Error bars indicate standard deviation (SD); n=4. **p<0.01, ****p<0.0001 according to 2-way ANOVA with Bonferroni's multiple comparison test.

FIGS. 5A-5E demonstrate zipIL-2R improves B7-H3-CAR T cell antitumor activity in vivo. (FIG. 5A) Experimental design. NSG mice were injected intravenously (i.v.) with 2×10⁶ A549 tumor cells in sterile PBS. After 7 days, 3×10⁵ CAR or CAR+zipIL-2R T cells were injected i.v. Tumor growth within the lungs was monitored by weekly bioluminescence imaging (BLI). The experiment was performed with two different T cell donors: (FIGS. 5B-5C) donor 1 (n=4-5 mice per group), and (FIGS. 5D-5E) donor 2 (n=5 mice per group); experiment ongoing. (FIG. 5B) Quantification of bioluminescence imaging (BLI) data corresponding to donor 1 group. (FIG. 5C) Kaplan Meier Survival curve; *p<0.05. (FIG. 5D) Quantification of bioluminescence imaging (BLI) data corresponding to donor group 2. (FIG. 5E) Bar graph showing statistical analysis of data shown in FIG. 5D focused on CAR vs CAR+zipTL-2R T cells; 2-way ANOVA; *p<0.05, **p<0.01, ****p<0.0001.

FIGS. 6A-6F show 2X zip receptor design can be re-engineered to provide IL-7 signaling to CAR T cells to improve antitumor activity in vitro. (FIG. 6A) Schematic representation of zipIL-7R. (FIG. 6B) Transduction efficiency and baseline pSTAT5 expression in human T cells; n=3-4. (FIG. 6C) Quantification of dead cells (Annexin V+ Dead+), early apoptotic cells (Annexin V+), necrotic cells (Dead+), and live cells (Annexin V− Dead−) in transduced human T cells after 21 days of cytokine starvation; n=3. (FIG. 6D) Transduction of human T cells with functional B7-H3-specific CAR (CAR), non-functional B7-H3-specific CAR (ΔCAR) and/or zipIL-7R; n=3. (FIG. 6E) 24-hour MTS cytotoxicity assay of CAR, CAR+zipIL-7R, ΔCAR, or ΔCAR+zipIL-7R T cells at different E:T with indicated cell lines; n=3. (FIG. 6F) Expansion of CAR, CAR+zipIL-7R, ΔCAR, or ΔCAR+zipIL-7R T cells after weekly serial co-culture with indicated tumor cell lines at a 2:1 E:T ratio; n=3. Error bars indicate SD; **p<0.01, ****p<0.0001.

FIGS. 7A-7D shows 2X zip receptor design can be re-engineered to provide IL-21 signaling to CAR T cells to improve antitumor activity in vitro. (FIG. 7A) Schematic representation of zipIL-21R. (FIG. 7B) Transduction efficiency and baseline pSTAT3 expression in human T cells; n=3 (FIG. 7C) Transduction of human T cells with functional B7-H3-specific CAR (CAR), non-functional B7-H3-specific CAR (ΔCAR) and/or zipIL-21R; n=2 (FIG. 7D) Expansion of CAR or CAR+zipIL-21R T cells after weekly serial co-culture with A549 WT cells at a 2:1 E:T ratio. Error bars indicate SD; *p<0.05.

FIGS. 8A-8C show 2X zip receptor expression in natural killer (NK) cells activates STAT5/3 signaling and improves survival. (FIG. 8A) Transduction of primary human NK cells with indicated zipReceptors. n=3; Error bars indicate SD. (FIG. 8B) Baseline pSTAT5 or pSTAT3 expression in transduced NK cells. n=3; Error bars indicate SD (FIG. 8C) Quantification of dead cells (Annexin V+ Dead+), early apoptotic cells (Annexin V+), necrotic cells (Dead+), and live cells (Annexin V− Dead−) in transduced human NK cells after seven days of cytokine starvation. n=3; Error bars indicate SD. **p<0.01, ***p<0.001, ****p<0.0001 according to 2-way ANOVA with Tukey's multiple comparison test.

FIGS. 9A-9B show exemplary zipIL-2R amino acid sequences. Exemplary AZip-EEX1 IL-2rb (IL-2 receptor beta [IL-2Rβ] chain) amino acid sequences (FIG. 9A; SEQ ID NO: 239). Exemplary BZip-RRx1 IL-2rg (IL-2 receptor gamma [IL-2Rγ] chain) amino acid sequences (FIG. 9B; SEQ ID NO: 240).

FIGS. 10A-10B show amino acid sequences (FIG. 10A; SEQ ID NO: 241) and nucleotide sequences (FIG. 10B; SEQ ID NO: 242) for an exemplary ZipTL-2R zipReceptor, comprising EE2Xzip, IL-2Rb, 2A, RR2Xzip, IL-2Rg, 2A and mClover.

FIGS. 11A-11B show amino acid sequences (FIG. 11A; SEQ ID NO: 243) and nucleotide sequences (FIG. 11B; SEQ ID NO: 244) for an exemplary ZipTL-7R zipReceptor, comprising EE2Xzip, IL-7Ra, 2A, RR2Xzip, IL-2Rg, 2A, and mClover

FIGS. 12A-12B show amino acid sequences (FIG. 12A; SEQ ID NO: 245) and nucleotide sequences (FIG. 12B; SEQ ID NO: 246) for an exemplary ZipIL-21R zipReceptor, comprising EE2Xzip, IL-21R, 2A, RR2Xzip, IL-2Rg, 2A, and mClover.

FIGS. 13A-13B show amino acid sequences (FIG. 13A; SEQ ID NO: 247) and nucleotide sequences (FIG. 13B; SEQ ID NO: 248) for an exemplary ZipTL-9R zipReceptor, comprising EE2Xzip, IL-9R, 2A, RR2Xzip, IL-2Rg, 2A, and mClover.

FIGS. 14A-14C show amino acid sequences (FIG. 14A; SEQ ID NO: 249) and nucleotide sequences (FIGS. 14B-14C; SEQ ID NO: 250) for an exemplary ZipTL-4R zipReceptor, comprising EE2Xzip, IL-4Ra, 2A, RR2Xzip, IL-2Rg, 2A, and mClover.

FIGS. 15A-15B show amino acid sequences (FIG. 15A; SEQ ID NO: 251) and nucleotide sequences (FIG. 15B; SEQ ID NO: 252) for an exemplary ZipIL-12R zipReceptor, comprising EE2Xzip, IL-12Rb1, 2A, RR2Xzip, IL-12Rb2, 2A, and mClover.

FIGS. 16A-16B show amino acid sequences (FIG. 16A; SEQ ID NO: 253) and nucleotide sequences (FIG. 16B; SEQ ID NO: 254) for an exemplary ZipIL-23R zipReceptor, comprising EE2Xzip, IL-12Rb1, 2A, RR2Xzip, IL-23R, 2A, and mClover.

FIGS. 17A-17B show amino acid sequences (FIG. 17A; SEQ ID NO: 255) and nucleotide sequences (FIG. 17B; SEQ ID NO: 256) for an exemplary ZipIL-1OR zipReceptor, comprising EE2Xzip, IL-1ORa, 2A, RR2Xzip, IL-10Rb, 2A, and mClover.

FIGS. 18A-18B show amino acid sequences (FIG. 18A; SEQ ID NO: 257) and nucleotide sequences (FIG. 18B; SEQ ID NO: 258) for an exemplary ZipIL-27R zipReceptor, comprising EE2Xzip, IL-6Rb, 2A, RR2Xzip, IL-27Ra, 2A, and mClover.

FIGS. 19A-19C show amino acid sequences (FIG. 19A; SEQ ID NO: 259) and nucleotide sequences (FIGS. 19B-19C; SEQ ID NO: 260) for an exemplary ZipIL-2/21R zipReceptor, comprising EE2Xzip, IL-2Rb, IL-21R, 2A, RR2Xzip, IL-2Rg, 2A, and mClover.

### DETAILED DESCRIPTION

Immunotherapy with immune cells including T cells expressing chimeric antigen receptors (CARs) has already revolutionized the treatment approach for patients with hematological malignancies. However, immune cells, including CAR T cells, have been less effective for solid tumors and brain tumors. While lack of efficacy is most likely multifactorial, the limited persistence without stimulation and an inability to function in the setting of chronic antigen exposure have emerged as major roadblocks.

To improve the ability of CAR T cells to remain viable without antigen exposure and function in the setting of chronic antigen exposure, investigators have overexpressed gamma cytokines, including IL-2, IL-7, IL-15, and IL-21, in CAR T cells. While gamma cytokine-expressing CAR T cells have improved antitumor activity in preclinical models, there are safety concerns since these secreted or membrane-bound cytokines have the potential to activate bystander immune cells. To overcome this limitation, the present application provides chimeric cytokine receptors that only provide signals to the genetically modified immune cells.

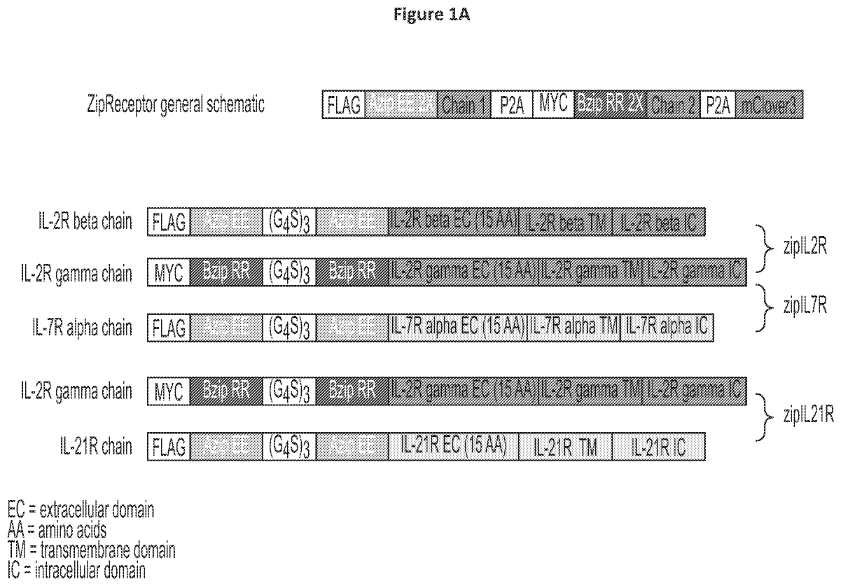

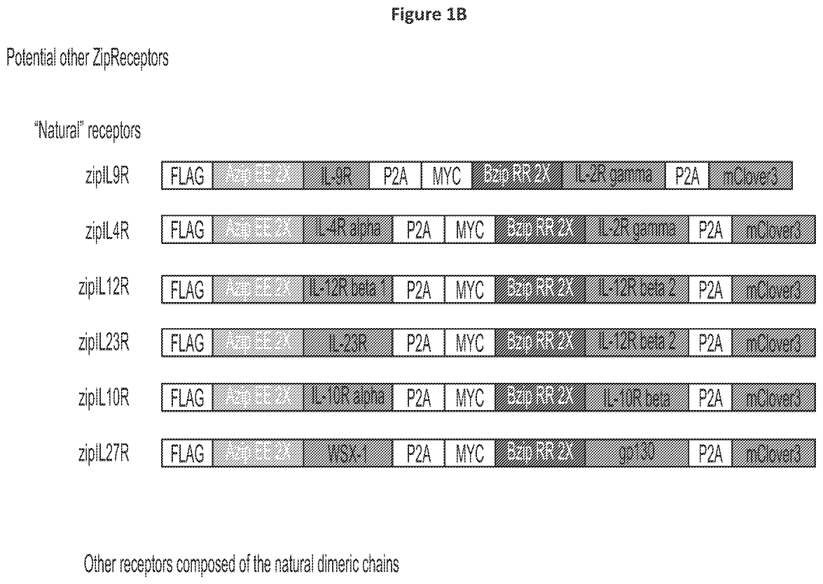

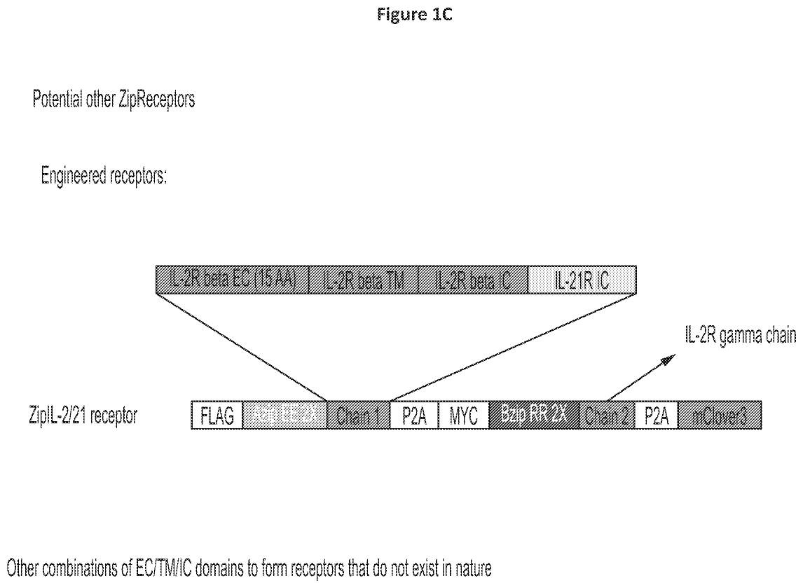

Gamma cytokine receptors are heterodimers, consisting of a common gamma chain and cytokine specific chains (except for IL-2 and IL-15, which share the same beta chain). To induce heterodimerization, the extracellular domain of the cytokine receptors was replaced with leucine zippers, therefore this novel class of receptors is termed “Zip Cytokine Receptors” (ZipRs or zipReceptors). A non-limiting example, ZipIL-2R (see FIG. 1A), was first generated. This receptor was demonstrated to be functional as judged by activation of downstream signaling molecules (phosphorylation of STAT5). Surprisingly, two leucine zipper motifs were needed to generate more active ZipRs, which could have not been predicted by the existing literature and/or experts in the field. ZipIL-2R T and CAR T cells remained viable in the absence of cytokine for at least one week. ZipIL-2R.CAR T cells retained their antigen specificity, and were able to sustain their effector function in repeat stimulation assays, mimicking chronic antigen exposure, in comparison to unmodified CAR T cells. In xenograft models, ZipIL-2R.CAR T cells also had improved anti-tumor activity in comparison to their unmodified counterparts.

In addition to ZipIL-2R, other two non-limiting examples, ZipIL-7Rs and ZipIL-21Rs, have also been generated (see FIG. 1A), and their functionality demonstrated. An additional list of exemplary zipReceptors have been designed as well (see FIGS. 1B and 1C). Thus, this application provides a modular chimeric cytokine receptor platform, which should have broad application to the entire field of adoptive cell therapies.

While data disclosed herein (see Examples section) are focused on expressing CAR in T cells, methods and/or compositions of the present disclosure may be applicable to various other cell therapy platforms that are actively being explored, including but not limited to, NK cells, NK92 cells, NKT cells, and γδ T cells. Likewise, while the methods and/or compositions disclosed herein may be useful in the treatment of cancer, the present invention may also be broadly applicable to adoptive immunotherapies of non-malignant disease including, but not limited to infectious diseases (e.g., viral infections) and autoimmune diseases.

### Definitions

The term “chimeric cytokine receptor” as used herein may refer to a cytokine receptor that is engineered to have at least a portion of at least one domain (e.g., extracellular region, transmembrane region, intracellular signaling region) that is derived from sequences of one or more different origins.

The term “chimeric antigen receptor” or “CAR” as used herein is defined as a cell-surface receptor comprising an extracellular target-binding domain, a transmembrane domain, and a cytoplasmic domain, comprising a lymphocyte activation domain and optionally at least one co-stimulatory signaling domain, all in a combination that is not naturally found together on a single protein. This particularly includes receptors wherein the extracellular domain and the cytoplasmic domain are not naturally found together on a single receptor protein. The chimeric antigen receptors of the present invention can be used with lymphocytes such as T-cells and natural killer (NK) cells.

The terms “T cell” and “T lymphocyte” are interchangeable and used synonymously herein. As used herein, T-cell includes thymocytes, naive T lymphocytes, immature T lymphocytes, mature T lymphocytes, resting T lymphocytes, or activated T lymphocytes. A T-cell can be a T helper (Th) cell, for example a T helper 1 (Th1) or a T helper 2 (Th2) cell. The T-cell can be a helper T-cell (HTL; CD4+ T-cell) CD4+ T-cell, a cytotoxic T-cell (CTL; CD8+ T-cell), a tumor infiltrating cytotoxic T-cell (TIL; CD8+ T-cell), CD4+ CD8+ T-cell, or any other subset of T-cells. Other illustrative populations of T-cells suitable for use in particular embodiments include naive T-cells and memory T-cells. Also included are “NKT cells”, which refer to a specialized population of T-cells that express a semi-invariant αβ T-cell receptor, but also express a variety of molecular markers that are typically associated with NK cells, such as NK1.1. NKT cells include NK1.1+ and NK1.1−, as well as CD4+, CD4−, CD8+ and CD8− cells. The TCR on NKT cells is unique in that it recognizes glycolipid antigens presented by the MHC I-like molecule CD Id. NKT cells can have either protective or deleterious effects due to their abilities to produce cytokines that promote either inflammation or immune tolerance. Also included are “gamma-delta T-cells (γδ T-cells),” which refer to a specialized population that to a small subset of T-cells possessing a distinct TCR on their surface, and unlike the majority of T-cells in which the TCR is composed of two glycoprotein chains designated a- and p-TCR chains, the TCR in γδ T-cells is made up of a γ-chain and a δ-chain. γδ T-cells can play a role in immunosurveillance and immunoregulation and were found to be an important source of IL-17 and to induce robust CD8+ cytotoxic T-cell response. Also included are “regulatory T-cells” or “Tregs” refers to T-cells that suppress an abnormal or excessive immune response and play a role in immune tolerance. Tregs cells are typically transcription factor Foxp3-positive CD4+ T cells and can also include transcription factor Foxp3-negative regulatory T-cells that are IL-10-producing CD4+ T cells.

The terms “natural killer cell” and “NK cell” are used interchangeably and used synonymously herein. As used herein, NK cell refers to a differentiated lymphocyte with a CD 16+ CD56+ and/or CD57+ TCR− phenotype. NKs are characterized by their ability to bind to and kill cells that fail to express “self” MHC/HLA antigens by the activation of specific cytolytic enzymes, the ability to kill tumor cells or other diseased cells that express a ligand for NK activating receptors, and the ability to release protein molecules called cytokines that stimulate or inhibit the immune response.

As used herein, the term “antigen” refers to any agent (e.g., protein, peptide, polysaccharide, glycoprotein, glycolipid, nucleic acid, portions thereof, or combinations thereof) molecule capable of being bound by a T-cell receptor. An antigen is also able to provoke an immune response. An example of an immune response may involve, without limitation, antibody production, or the activation of specific immunologically competent cells, or both. A skilled artisan will understand that an antigen need not be encoded by a “gene” at all. It is readily apparent that an antigen can be generated synthesized or can be derived from a biological sample or might be a macromolecule besides a polypeptide. Such a biological sample can include, but is not limited to a tissue sample, a tumor sample, a cell or a fluid with other biological components, organisms, subunits of proteins/antigens, killed or inactivated whole cells or lysates.

The term “antigen-recognition molecule” refers to any molecule that is capable of recognizing an antigen as described herein. Non-limiting examples of antigen-recognition molecules include T cell receptors (TCRs) (e.g., αβ TCRs), synthetic T cell receptors and antigen receptors (STARs), chimeric antigen receptor (CARs), T cell antigen couplers (TACs), T cell receptor fusion constructs (TruCs), antibodies (e.g., bispecific antibodies) or antibody fragments.

Terms “antibody” and “antibodies” refer to monoclonal antibodies, multispecific antibodies (e.g., bispecific antibodies), human antibodies, humanized antibodies, chimeric antibodies, single-chain Fvs (scFv), single chain antibodies, Fab fragments, F(ab′) fragments, disulfide-linked Fvs (sdFv), intrabodies, minibodies, diabodies and anti-idiotypic (anti-Id) antibodies (including, e.g., anti-Id antibodies to antigen specific TCR), and epitope-binding fragments of any of the above. The terms “antibody” and “antibodies” also refer to covalent diabodies such as those disclosed in U.S. Pat. Appl. Pub. 2007/0004909 and Ig-DARTS such as those disclosed in U.S. Pat. Appl. Pub. 2009/0060910. Antibodies useful as a TCR-binding molecule include immunoglobulin molecules and immunologically active fragments of immunoglobulin molecules, i.e., molecules that contain an antigen-binding site. Immunoglobulin molecules can be of any type (e.g., IgG, IgE, IgM, IgD, IgA and IgY), class (e.g., IgG1, IgG2, IgG3, IgG4, IgM1, IgM2, IgA1 and IgA2) or subclass.

The term “host cell” means any cell that contains a heterologous nucleic acid. The heterologous nucleic acid can be a vector (e.g., an expression vector). For example, a host cell can be a cell from any organism that is selected, modified, transformed, grown, used or manipulated in any way, for the production of a substance by the cell, for example the expression by the cell of a gene, a DNA or RNA sequence, a protein or an enzyme. An appropriate host may be determined. For example, the host cell may be selected based on the vector backbone and the desired result. By way of example, a plasmid or cosmid can be introduced into a prokaryote host cell for replication of several types of vectors. Bacterial cells such as, but not limited to DH5a, JM109, and KCB, SURE® Competent Cells, and SOLOPACK Gold Cells, can be used as host cells for vector replication and/or expression. Additionally, bacterial cells such as E. coli LE392 could be used as host cells for phage viruses. Eukaryotic cells that can be used as host cells include, but are not limited to yeast (e.g., YPH499, YPH500 and YPH501), insects and mammals. Examples of mammalian eukaryotic host cells for replication and/or expression of a vector include, but are not limited to, HeLa, NIH3T3, Jurkat, 293, COS, CHO, Saos, and PC12. In certain embodiments, the host cell is autologous. In certain embodiments, the host cell is allogenic.

Host cells of the present disclosure include immune cells (e.g., T-cells and natural killer cells, or a macrophage) or stem cells (e.g., mesenchymal stem cells (MSCs), induced pluripotent stem (iPS) cells) that contain the DNA or RNA sequences encoding a chimeric cytokine receptor described herein and express the chimeric cytokine receptor on the cell surface. Host cells may be used for enhancing immune cell activity (e.g., effector function), treatment of tumors, treatment of infectious diseases, and treatment of autoimmune disease.

The terms “activation” or “stimulation” means to induce a change in their biologic state by which the cells (e.g., T-cells and NK cells) express activation markers, produce cytokines, proliferate, and/or become cytotoxic to target cells. All of these changes can be produced by primary stimulatory signals. Co-stimulatory signals can amplify the magnitude of the primary signals and suppress cell death following initial stimulation, resulting in a more durable activation state and thus a higher cytotoxic capacity. A “co-stimulatory signal” refers to a signal, which in combination with a primary signal, such as TCR/CD3 ligation, leads to T-cell and/or NK cell proliferation and/or upregulation or downregulation of key molecules.

The terms “express” and “expression” mean allowing or causing the information in a gene or DNA sequence to become produced, for example producing a protein by activating the cellular functions involved in transcription and translation of a corresponding gene or DNA sequence. A DNA sequence is expressed in or by a cell to form an “expression product” such as a protein. The expression product itself, e.g., the resulting protein, may also be said to be “expressed” by the cell. An expression product can be characterized as intracellular, extracellular or transmembrane.

The term “tumor” refers to a benign or malignant abnormal growth of tissue. The term “tumor” includes cancer.

The term “effector function” refers to a specialized function of a cell. Effector function of a T cell, for example, may be cytolytic activity (e.g., tumor killing activity) or helper activity including the secretion of cytokines.

As used herein, the term “safety switch” refers to any mechanism that is capable of removing or inhibiting the effect of a chimeric cytokine receptor described herein from a system (e.g., a culture or a subject).

The term “site-specific nuclease” as used herein refers to a nuclease capable of specifically recognizing and cleaving a nucleic acid (DNA or RNA) sequence.

The terms “genetically modified” or “genetically engineered” refers to the addition of extra genetic material in the form of DNA or RNA into a cell.

As used herein, the term “derivative” or “derived from” in the context of proteins or polypeptides (e.g., chimeric cytokine receptors or domains thereof) refer to: (a) a polypeptide that has at least 40%, 45%, 50%, 55%, 60%, 65%, 70%, 75%, 80%, 85%, 90%, 95%, 98% or 99% sequence identity to the polypeptide it is a derivative of; (b) a polypeptide encoded by a nucleotide sequence that has at least 40%, 45%, 50%, 55%, 60%, 65%, 70%, 75%, 80%, 85%, 90%, 95%, 98% or 99% sequence identity to a nucleotide sequence encoding the polypeptide it is a derivative of; (c) a polypeptide that contains 1, 2, 3, 4, 5, 6, 7, 8, 9, 10, 11, 12, 13, 14, 15, 16, 17, 18, 19, 20 or more amino acid mutations (i.e., additions, deletions and/or substitutions) relative to the polypeptide it is a derivative of; (d) a polypeptide encoded by a nucleotide sequence that can hybridize under high, moderate or typical stringency hybridization conditions to nucleic acids encoding the polypeptide it is a derivative of; (e) a polypeptide encoded by a nucleotide sequence that can hybridize under high, moderate or typical stringency hybridization conditions to a nucleotide sequence encoding a fragment of the polypeptide, it is a derivative of, of at least 20 contiguous amino acids, at least 30 contiguous amino acids, at least 40 contiguous amino acids, at least 50 contiguous amino acids, at least 75 contiguous amino acids, at least 100 contiguous amino acids, at least 125 contiguous amino acids, or at least 150 contiguous amino acids; or (f) a fragment of the polypeptide it is a derivative of.

Percent sequence identity can be determined using any method known to one of skill in the art. In a specific embodiment, the percent identity is determined using the “Best Fit” or “Gap” program of the Sequence Analysis Software Package (Version 10; Genetics Computer Group, Inc., University of Wisconsin Biotechnology Center, Madison, Wisconsin). Information regarding hybridization conditions (e.g., high, moderate, and typical stringency conditions) have been described, see, e.g., U.S. Patent Application Publication No. US 2005/0048549 (e.g., paragraphs 72-73).

The term “functional fragment” as used herein refers to a fragment of the polypeptide or protein, or a polynucleotide encoding the polypeptide or protein, that retains at least one function of the full-length polypeptide or protein. A functional fragment may comprise one, two, three, or more fragments of the full-length polypeptide or protein, or polynucleotide encoding the polypeptide or protein. Each fragment may comprise an amino acid sequence of at least 5 contiguous amino acid residues, at least 6 contiguous amino acid residues, at least 7 contiguous amino acid residues, at least 8 contiguous amino acid residues, at least 9 contiguous amino acid residues, at least 10 contiguous amino acid residues, at least 11 contiguous amino acid residues, at least 12 contiguous amino acid residues, at least 13 contiguous amino acid residues, at least 14 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least contiguous 80 amino acid residues, at least contiguous 90 amino acid residues, at least contiguous 100 amino acid residues, at least contiguous 125 amino acid residues, at least 150 contiguous amino acid residues, at least contiguous 175 amino acid residues, at least contiguous 200 amino acid residues, or at least contiguous 250 amino acid residues of the amino acid sequence of the full-length polypeptide or protein.

The terms “vector”, “cloning vector,” “recombinant vector,” and “expression vector” mean the vehicle by which a DNA or RNA sequence (e.g., a foreign gene) can be introduced into a host cell, so as to genetically modify the host and promote expression (e.g., transcription and translation) of the introduced sequence. Vectors include plasmids, synthesized RNA and DNA molecules, phages, viruses, etc. In certain embodiments, the vector is a viral vector such as, but not limited to, viral vector is an adenoviral, adeno-associated, alphaviral, herpes, lentiviral, retroviral, or vaccinia vector.

As used herein, the term “operably linked,” or “operatively linked,” and similar phrases, when used in reference to nucleic acids or amino acids, refer to the operational linkage of nucleic acid sequences or amino acid sequence, respectively, placed in functional relationships with each other. For example, an operatively linked promoter, enhancer elements, open reading frame, 5′ and 3′ UTR, and terminator sequences result in the accurate production of a nucleic acid molecule (e.g., RNA). In some embodiments, operatively linked nucleic acid elements result in the transcription of an open reading frame and ultimately the production of a polypeptide (i.e., expression of the open reading frame). As another example, an operatively linked peptide is one in which the functional domains are placed with appropriate distance from each other to impart the intended function of each domain.

As used herein, the term “promoter” refers to any sequence(s) capable of driving transcription of a coding sequence in a cell. Therefore, promoters used in the vectors of the disclosure may comprise cis-acting transcriptional control elements and regulatory sequences that are involved in regulating or modulating the timing and/or rate of transcription of a gene. By way of a non-limiting example, a promoter can be a cis-acting transcriptional control element, including an enhancer, a promoter, a transcription terminator, an origin of replication, a chromosomal integration sequence, 5′ and 3′ untranslated regions, or an intronic sequence, which participate in transcriptional regulation. Such cis-acting sequences typically interact with proteins or other biomolecules to carry out (turn on/off, regulate, modulate, etc.) transcription. “Constitutive” promoters are those that drive expression continuously under the majority of environmental conditions and states of cellular development and/or differentiation. “Inducible” or “regulatable” promoters direct expression of a nucleic acid under the influence of developmental and/or environmental. Non-limiting examples of environmental conditions that may influence transcription via inducible promoters include elevated temperature, drought, the presence of light, and anaerobic conditions.

By “enhance” or “promote,” or “increase” or “expand” or “improve” refers generally to the ability of a composition contemplated herein to produce, elicit, or cause a greater physiological response (i.e., downstream effects) compared to the response caused by either vehicle or a control molecule/composition. A measurable physiological response may include an increase in immune cell expansion, activation, effector function, persistence, and/or an increase in tumor cell death killing ability, among others apparent from the understanding in the art and the description herein. In certain embodiments, an “increased” or “enhanced” amount can be a “statistically significant” amount, and may include an increase that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response produced by vehicle or a control composition.

By “decrease” or “lower,” or “lessen,” or “reduce,” or “abate” refers generally to the ability of composition contemplated herein to produce, elicit, or cause a lesser physiological response (i.e., downstream effects) compared to the response caused by either vehicle or a control molecule/composition. In certain embodiments, a “decrease” or “reduced” amount can be a “statistically significant” amount, and may include a decrease that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response (reference response) produced by vehicle, a control composition, or the response in a particular cell lineage.

The terms “treat” or “treatment” of a state, disorder or condition include: (1) preventing, delaying, or reducing the incidence and/or likelihood of the appearance of at least one clinical or sub-clinical symptom of the state, disorder or condition developing in a subject that may be afflicted with or predisposed to the state, disorder or condition, but does not yet experience or display clinical or subclinical symptoms of the state, disorder or condition; or (2) inhibiting the state, disorder or condition, i.e., arresting, reducing or delaying the development of the disease or a relapse thereof or at least one clinical or sub-clinical symptom thereof, or (3) relieving the disease, i.e., causing regression of the state, disorder or condition or at least one of its clinical or sub-clinical symptoms. The benefit to a subject to be treated is either statistically significant or at least perceptible to the patient or to the physician.

The term “effective” applied to dose or amount refers to that quantity of a compound or pharmaceutical composition that is sufficient to result in a desired activity upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

The term “pharmaceutical composition,” as used herein, represents a composition comprising polynucleotides, vectors, peptides, compositions, or host cells described herein formulated for administration to a subject for treatment, abatement, or prevention of a disease.

The phrase “pharmaceutically acceptable”, as used in connection with compositions described herein, refers to molecular entities and other ingredients of such compositions that are physiologically tolerable and do not typically produce untoward reactions when administered to a mammal (e.g., a human). Preferably, the term “pharmaceutically acceptable” means approved by a regulatory agency of the Federal or a state government or listed in the U.S. Pharmacopeia or other generally recognized pharmacopeia for use in mammals, and more particularly in humans.

The term “protein” is used herein encompasses all kinds of naturally occurring and synthetic proteins, including protein fragments of all lengths, fusion proteins and modified proteins, including without limitation, glycoproteins, as well as all other types of modified proteins (e.g., proteins resulting from phosphorylation, acetylation, myristoylation, palmitoylation, glycosylation, oxidation, formylation, amidation, polyglutamylation, ADP-ribosylation, pegylation, biotinylation, etc.).

The terms “nucleic acid”, “nucleotide”, and “polynucleotide” encompass both DNA and RNA unless specified otherwise. By a “nucleic acid sequence” or “nucleotide sequence” is meant the nucleic acid sequence encoding an amino acid, the term may also refer to the nucleic acid sequence including the portion coding for any amino acids added as an artifact of cloning, including any amino acids coded for by linkers.

The terms “patient”, “individual”, “subject”, and “animal” are used interchangeably herein and refer to mammals, including, without limitation, human and veterinary animals (e.g., cats, dogs, cows, horses, sheep, pigs, etc.) and experimental animal models. In a preferred embodiment, the subject is a human.

The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Alternatively, the carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E. W. Martin.

Singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure.

The term “about” or “approximately” includes being within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

If aspects of the disclosure are described as “comprising”, or versions thereof (e.g., comprises), a feature, embodiments also are contemplated “consisting of” or “consisting essentially of” the feature.

The practice of the present disclosure employs, unless otherwise indicated, conventional techniques of statistical analysis, molecular biology (including recombinant techniques), microbiology, cell biology, and biochemistry, which are within the skill of the art. Such tools and techniques are described in detail in e.g., Sambrook et al. (2001) Molecular Cloning: A Laboratory Manual. 3rd ed. Cold Spring Harbor Laboratory Press: Cold Spring Harbor, New York; Ausubel et al. eds. (2005) Current Protocols in Molecular Biology. John Wiley and Sons, Inc.: Hoboken, NJ; Bonifacino et al. eds. (2005) Current Protocols in Cell Biology. John Wiley and Sons, Inc.: Hoboken, NJ; Coligan et al. eds. (2005) Current Protocols in Immunology, John Wiley and Sons, Inc.: Hoboken, NJ; Coico et al. eds. (2005) Current Protocols in Microbiology, John Wiley and Sons, Inc.: Hoboken, NJ; Coligan et al. eds. (2005) Current Protocols in Protein Science, John Wiley and Sons, Inc.: Hoboken, NJ; and Enna et al. eds. (2005) Current Protocols in Pharmacology, John Wiley and Sons, Inc.: Hoboken, NJ. Additional techniques are explained, e.g., in U.S. Pat. No. 7,912,698 and U.S. Patent Appl. Pub. Nos. 2011/0202322 and 2011/0307437.

The technology illustratively described herein suitably may be practiced in the absence of any element(s) not specifically disclosed herein.

The terms and expressions which have been employed are used as terms of description and not of limitation, and use of such terms and expressions do not exclude any equivalents of the features shown and described or portions thereof, and various modifications are possible within the scope of the technology claimed.

### Chimeric Cytokine Receptor

In some embodiments, the present disclosure provides a chimeric cytokine receptor comprising
a) a first polypeptide comprising:
   i) a first extracellular region comprising one or more leucine zipper motifs,
   ii) optionally, a first hinge region,
   iii) a first transmembrane region, and
   iv) at least a first intracellular signaling region derived from a first cytokine receptor chain; and
b) a second polypeptide comprising:
   i) a second extracellular region comprising one or more leucine zipper motifs,
   ii) optionally, a second hinge region,
   iii) a second transmembrane region, and
   iv) at least a second intracellular signaling region derived from a second cytokine receptor chain.

In some embodiments, the one or more leucine zipper motifs from the first extracellular region heterodimerize with the one or more leucine zipper motifs from the second extracellular region.

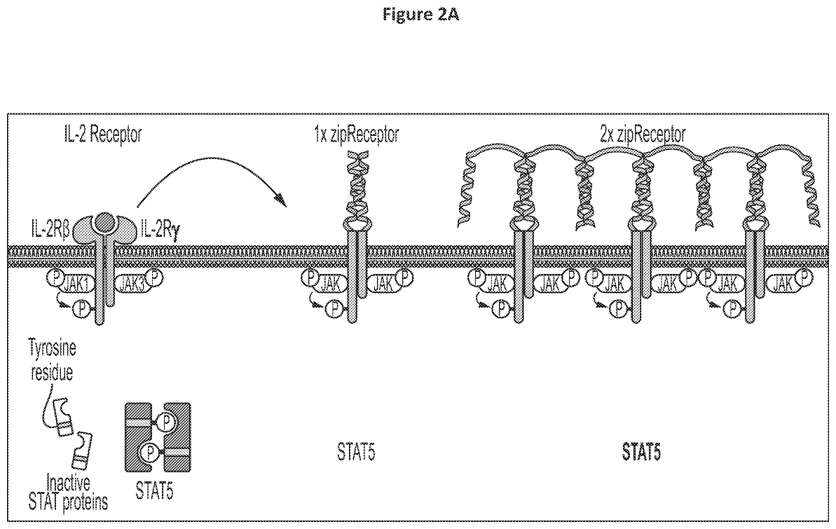

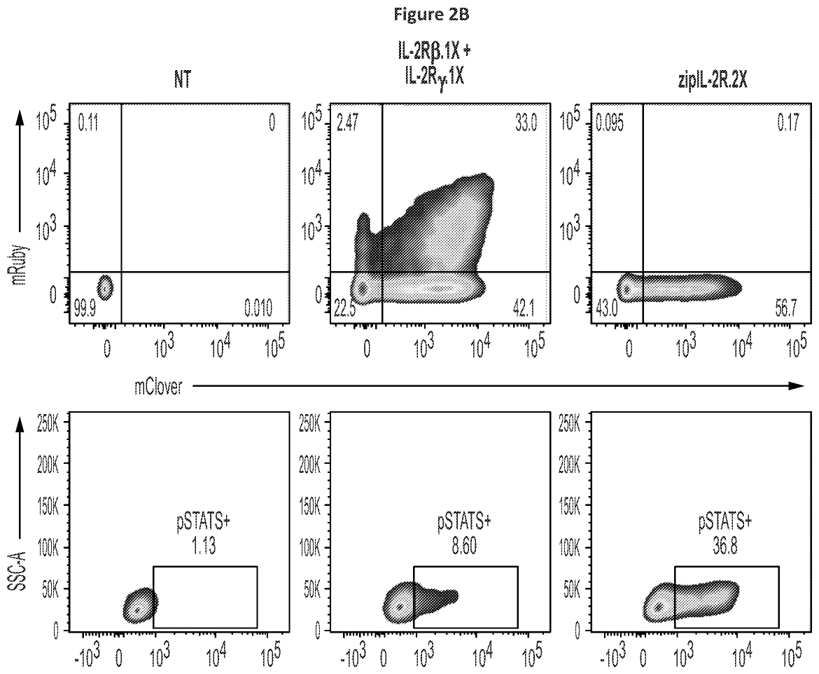

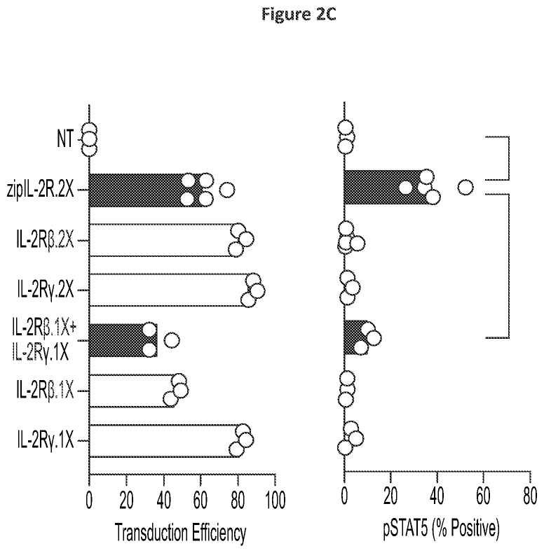

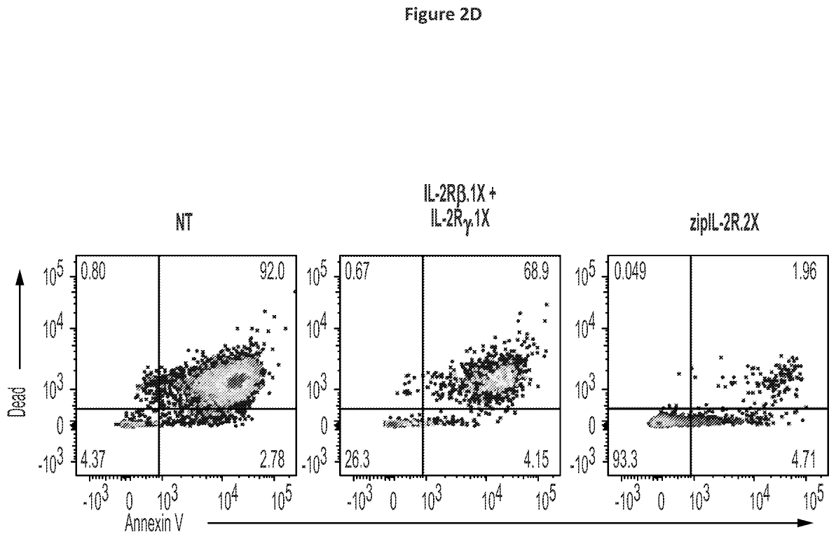

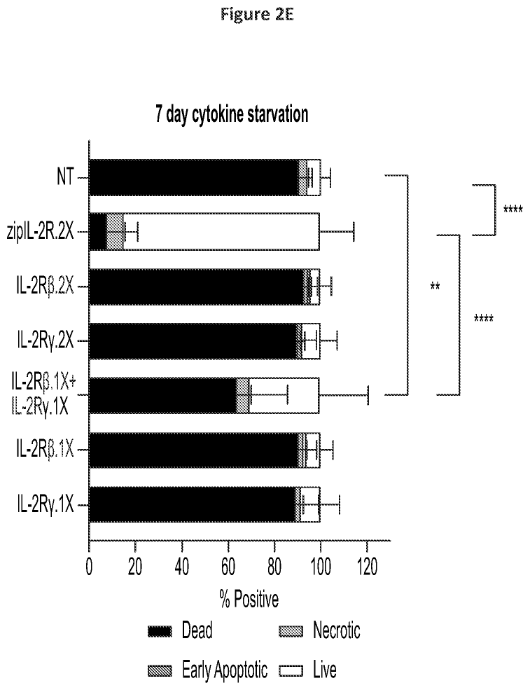

The leucine zipper motifs of the first polypeptide and the second polypeptide can interact in a parallel or antiparallel alignment. In some cases, some of the leucine zipper motifs of a chimeric cytokine receptor may interact with leucine zipper motifs from a different chimeric cytokine receptor when the two chimeric cytokine receptors are positioned in close proximity to each other. In some embodiments, the leucine zipper motifs enable oligomerization of the chimeric cytokine receptors, see FIG. 2A for example.

### Extracellular Region

In some embodiments, the chimeric cytokine receptor disclosed herein may comprise a first polypeptide comprising a first extracellular region. In some embodiments, the chimeric cytokine receptor disclosed herein may comprise a second polypeptide comprising a second extracellular region.

In some embodiments, the first and/or second extracellular region may comprise one or more leucine zipper motifs.

In some embodiments, each of the first and/or second extracellular regions of any of the chimeric cytokine receptors of the present disclosure may comprise one leucine zipper motif. In some embodiments, each extracellular region may comprise two or more leucine zipper motifs. As a non-limiting example, each extracellular region may comprise up to 1, 2, 3, 4, 5, 6, 7, 9, or 10 leucine zipper motifs.

In some embodiments, each of the leucine zipper motifs comprising the first or second extracellular region disclosed herein may comprise at least five heptad repeats of amino acids with a leucine at every seventh position as described, for example, in Seldeen et al., 2010 [40], the contents of which is incorporated herein by reference in its entirety for all purposes.

In some embodiments, each leucine zipper motif of the first extracellular region may comprise the amino acid sequence of SEQ ID NO: 1, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 1. In some embodiments, the nucleotide sequence that encodes leucine zipper motif of the first extracellular region comprises the nucleotide sequence that encodes the amino acid sequence of SEQ ID NO: 1, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 1. In some embodiments, each leucine zipper motif of the first extracellular region may comprises the amino acid sequence set forth in SEQ ID NO: 1.

In some embodiments, each leucine zipper motif of the first extracellular region may comprise the amino acid sequence of SEQ ID NO: 2, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 2. In some embodiments, the nucleotide sequence that encodes leucine zipper motif of the first extracellular region comprises the nucleotide sequence that encodes the amino acid sequence of SEQ ID NO: 2, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 2. In some embodiments, each leucine zipper motif of the first extracellular region may comprises the amino acid sequence set forth in SEQ ID NO: 2.

In some embodiments, each leucine zipper motif of the second extracellular region may comprise the amino acid sequence of SEQ ID NO: 1, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 1. In some embodiments, the nucleotide sequence that encodes leucine zipper motif of the second extracellular region comprises the nucleotide sequence that encodes the amino acid sequence of SEQ ID NO: 1, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 1. In some embodiments, each leucine zipper motif of the second extracellular region may comprises the amino acid sequence set forth in SEQ ID NO: 1.

In some embodiments, each leucine zipper motif of the second extracellular region may comprise the amino acid sequence of SEQ ID NO: 2, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 2. In some embodiments, the nucleotide sequence that encodes leucine zipper motif of the second extracellular region comprises the nucleotide sequence that encodes the amino acid sequence of SEQ ID NO: 2, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 2. In some embodiments, each leucine zipper motif of the second extracellular region may comprises the amino acid sequence set forth in SEQ ID NO: 2.

In some embodiments, any of the chimeric cytokine receptor disclosed here may comprise a first and/or a second extracellular region comprising, e.g., one or more leucine zipper motifs, wherein a) each leucine zipper motif of the first extracellular region may comprise the amino acid sequence set forth in SEQ ID NO: 1, or variants thereof, and each leucine zipper motif of the second extracellular region may comprise the amino acid sequence set forth in SEQ ID NO: 2, or variants thereof; or b) each leucine zipper motif of the first extracellular region may comprise the amino acid sequence set forth in SEQ ID NO: 2, or variants thereof, and each leucine zipper motif of the second extracellular region may comprise the amino acid sequence set for in SEQ ID NO: 1, or variants thereof.

In some embodiments, the at least two leucine zipper motifs comprises the amino acid sequence of SEQ ID NO: 8, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 8. In some embodiments, the nucleotide sequence that encodes the at least two leucine zipper motifs comprises the nucleotide sequence that encodes the amino acid sequence of SEQ ID NO: 8, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 8 In some embodiments, the nucleotide sequence that encodes the at least two leucine zipper motifs comprises the nucleotide sequence set forth in SEQ ID NO: 9, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 9. In some embodiments, the nucleotide sequence that encodes the at least two leucine zipper motifs comprises the nucleotide sequence set forth in SEQ ID NO: 10, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO:10. In some embodiments, the at least two leucine zipper motifs comprises the amino acid sequence set forth in SEQ ID NO: 8. In some embodiments, the nucleotide sequence that encodes at least two leucine zipper motifs comprises the nucleotide sequence set forth in SEQ ID NO: 9. In some embodiments, the nucleotide sequence that encodes at least two leucine zipper motifs comprises the nucleotide sequence set forth in SEQ ID NO: 10.

In some embodiments, the at least two leucine zipper motifs comprises the amino acid sequence of SEQ ID NO: 27, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 27. In some embodiments, the nucleotide sequence that encodes the at least two leucine zipper motifs comprises the nucleotide sequence that encodes the amino acid sequence of SEQ ID NO: 27, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 27. In some embodiments, the nucleotide sequence that encodes the at least two leucine zipper motifs comprises the nucleotide sequence set forth in SEQ ID NO: 28, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 28. In some embodiments, the nucleotide sequence that encodes the at least two leucine zipper motifs comprises the nucleotide sequence set forth in SEQ ID NO: 29, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 29. In some embodiments, the at least two leucine zipper motifs comprises the amino acid sequence set forth in SEQ ID NO: 27. In some embodiments, the nucleotide sequence that encodes at least two leucine zipper motifs comprises the nucleotide sequence set forth in SEQ ID NO: 28. In some embodiments, the nucleotide sequence that encodes at least two leucine zipper motifs comprises the nucleotide sequence set forth in SEQ ID NO: 29.

In some embodiments, the at least two leucine zipper motifs may comprise any such motif as described, for example, in Moll et al., 2001 [41], Reike et al. 2013 [42], and Newman et al., 2003 [43], the contents of each of which is incorporated herein by reference in its entirety for all purposes.

In certain embodiments, zipper motifs, e.g., leucine zipper motifs, that may be used in accordance with the present disclosure may be derived from any number of human proteins within the knowledge of one skilled in the art. Non-limiting examples of human proteins comprising zipper motifs that may be used in accordance with the present disclosure are described in Table 1. Regarding Table 1 (see also Newman JRS and Keating AE. Comprehensive identification of human bZIP interactions with coiled-coil arrays. Science. 2003 Jun. 27; 300(5628):2097-101, incorporated herein by reference in its entirety for all intended purposes), a: Other names for a protein specified at left, or names of other proteins sharing the same coiledcoil sequence used here. b: Entries are omitted for sequences not present in the screen. AA: amino acid. c: The sequences used in the array experiments are divided into five sections. From left to right: [cloning vector sequence]-[basic region included for cloning]-[coiled-coil domain]-[additional resides to facilitate cloning]-[cloning vector]. The N-terminal end of the coiled-coil domain (at left) was determined by reference to the Fos/Jun crystal structure, the C-terminal end by the point at which the

TABLE 1
Non-Limiting Examples of Human Proteins Comprising Zipper Motifs

| | | | | SEQ |
|---|---|---|---|---|
| | | | | ID |
| Family | Name | Aliasesª | Protein Construct^{c} | NO. |
| ATF-2 | ATF-2 | CREB-2, CREBP1, HB16, | [2]-DLERNRAAASRCRQKRK- | 162 |
| | | TCRATF2 | VWVQSLEKKAEDLSSLNGQLQSEVTLLRNEVAQLKQLLLAHKDC-PVTAMQKKSG- | |
| | | | [10] | |
| ATF-2 | ATF-7 | ATFa | [2]-KRK-LWVSSLEKKAEELTSQNIQLSNEVTLLRNEVAQLKQLLLAHKDC- | 163 |
| | | | PVTALQK-[5] | |
| ATF-2 | CREBPA | CBREPA | [2]-QRK-VWVMSLEKKAEELTQTNMQLQNEVSMLKNEVAQLKQLLLTHKDC- | 164 |
| | | | PITAMQKES-[5] | |
| ATF-3 | ATF-3 | LRF-1 | [1]-CPEEDERKKRRRERNKIAAAKCRNKKK- | 165 |
| | | | EKTECLQKESEKLESVNAELKAQIEELKNEKQHLIYMLNLHR- | |
| | | | PTCIVRAQNGRTPED-[5] | |
| ATF-3 | JDP2 | — | ERTEFLQRESERLELMNAELKTQIEELKQERQQLILMLNRHR | 166 |
| ATF-4 | ATF-4 | TAXREB67,CREB2 | [2]-NKTAATRYRQKKR- | 167 |
| | | | AEQEALTGECKELEKKNEALKERADSLAKEIQYLKDLIEEVRKARGKKRV-P-[] | |
| ATF-4 | ATF-4L1^{d} | similar to ATF4 | [2]-KKGTRYRQKKR- | 168 |
| | | | VEQEVLTGECKAVEKKNEALQERADSLAEEIQYMKDSIEEVCKARGK-KRVL-[] | |
| ATF-4 | ATF-5 | ATFx | [2]-NRKQKKRDQNKSAALRYRQRKR- | 169 |
| | | | AEGEALEGECQGLEARNRELKERAESVEREIQYVKDLLIEVYKARSQ-RTRSC-[] | |
| ATF-6 | ATF-6 | ATF6alpha | [2]-ACQSRKKKK-EYMLGLEARLKAALSENEQLKKENGTLKRQLDEVVSENQRLKV- | 170 |
| | | | PSPKRRV-[5] | |
| ATF-6 | CREBL1 | ATF6beta, CREB-RP | EYLQGLEARLQAVLADNQQLRRENAALRRRLEALLAENSELKLGSGNRK | 171 |
| ATF-6 | ZF | Zhangfei | [2]-CRLNRLKKK- | 172 |
| | | | EYVMGLESRVRGLAAENQELRAENRELGKRVQALQEESRYLRAVLANETGL-[]-[9] | |
| B-ATF | B-ATF | p21-SNFT | [1]-[]-QKADTLHLESEDLEKQNAALRKEIKQLTEELKYFTSVLNSHE-[]-[5] | 173 |
| B-ATF | p21SNFT | — | [2]-RSRKKQT-QKADKLHEEYESLEQENTMLRREIGKLTEELKHLTEALKEHEKMC- | 174 |
| | | | PLLLCPMNFVH-[5] | |
| B-ATF | BAC04846 | — | DKADALHQQHESLEKDNLALRKEIQSLQAELAWWSRTLHVHE | 175 |
| C/EBP | C/EBPα | C/EBP | [1]-[]-QRNVETQQKVLELTSDNDRLRKRVEQLSRELDTLRGIFRQL-[]-[5] | 176 |
| C/EBP | C/EBPβ | NF-IL6, TF 5, LAP, | [2]-SDEYKIRRERNNIAVRKSRDKAK- | 177 |
| | | CRP2, LIP, AGP/EBP, | MRNLETQHKVLELTAENERLQKKVEQLSRELSTLRNLFKQL-PEPLLASSGHC-[] | |
| | | FN-M, GPE1-BP | | |
| C/EBP | C/EBPγ | Ig/EBP-1 | [2]-GERNNMAVKKSRLKSK- | 178 |
| | | | QKAQDTLQRVNQLKEENERLEAKIKLLTKELSVLKDLFLEHAHNLAD- | |
| | | | NVQSISTENTTADG-[5] | |
| C/EBP | C/EBPδ | NFIL6, NFIL6beta | [1]-[]-RRNQEMQQKLVELSAENEKLHQRVEQLTRDLAGLRQFFKQL-[]-[5] | 179 |
| C/EBP | C/EBPϵ | — | [2]-HLEYRLRRERNNIAVRKSRDKAK- | 180 |
| | | | RRILETQQKVLEYMAENERLRSRVEQLTQELDTLRNLFRQI-PEAANLIKGV-[5] | |
| C/EBP | CHOP | DDIT3, GADD153 | [2]-RMK-EKEQENERKVAQLAEENERLKQEIERLTREVEATRRALIDRMVNLHQA-[]- | 181 |
| | | | [] | |
| C/EBP | HP8 | NFIL3 | [1]-[]-QRNVETQQKVLELTSDNDRLRNGVEQLSRELDTLRGIFRQL-[]-[5] | 182 |
| CNC | NFE2 | p45 NF-E2 | [2]-QRKL-ETIVQLERELERLTNERERLLRARGEADRTLEVMRQQLTELYR- | 183 |
| | | | DIFQHLRDESGNS-[] | |
| CNC | NFE2L1 | NRF1, TCF11, HBZ17, | [2]-KL-DTILNLERDVEDLQRDKARLLREKVEFLRSLRQMKQKVQSLYQEV- | 184 |
| | | LCR-F1 | FGRLRDENGRPY-[11] | |
| CNC | NFE2L2 | NRF2,HEBP1 | [2]-FRDIRRRGKNKVAAQNCRKRKL- | 185 |
| | | | ENIVELEQDLDHLKDEKEKLLKEKGENDKSLHLLKKQLSTLYLEV-FSMLRD-[12] | |
| CNC | NFE2L3 | | [2]-GDIRRRGKNKVAAQNCRKRKL- | 186 |
| | | | DIILNLEDDVCNLQAKKETLKREQAQCNKAINIMKQKLHDL- | |
| | | | YHDIFSRLRDDQGRPV-[5] | |
| CNC | BACH1 | HA2303 | [2]-GCRKRKL-DCIQNLESEIEKLQSEKESLLKERDHILSTLGETKQNLTGL- | 187 |
| | | | CQKVCKEAALSQEQ-[13] | |
| CNC | BACH2 | | [2]-RKRKL-DCIQNLECEIRKLVCEKEKLLSERNQLKACMGEL- | 188 |
| | | | LDNFSCLSQEVCRDIQ-[5] | |
| CREB | ATF-1 | TREB36, TREB, TCRATF1 | [2]-DPQLKREIRLMKNREAARECRRKKK- | 189 |
| | | | EYVKCLENRVAVLENQNKTLIEELKTLKDLYSNKSV-[]-[] | |
| CREB | CREM-Ia | HCREM-1; CREMα, τα; | [1]-KEEATRKRELRLMKNREAARECRRKKK- | 190 |
| | | ICER1, 1γ | EYVKCLENRVAVLENQNKTLIEELKALKDLYCHKVE-[]-[] | |
| CREB | CREM- | possible sequence variant | [1]-KEEATRKRELRLMKNREAARECRRKKK- | 191 |
| | Ia(K324R) | (AAC60616 numbering) | EYVKCLENRVAVLENQNKTLIEELKALRDLYC-[]-[7] | |
| CREB | CREM-Ib | CREMβ, γ, τ, τ1, τ2, τα; | [1]-[]-EYVKCLESRVAVLEVQNKKLIEELETLKDICS-[]-[5] | 192 |
| | | ICER1, 1γ, 2γ, 3 | | |
| CREB | CREB | CREBδ, CREB-A, | [1]-KEAARKREVRLMKNREAARECRRKKK- | 193 |
| | | CREB327, CREB-1 | EYVKCLENRVAVLENQNKTLIEELKALKDLYCHKSD-[]-[] | |
| E4BP4 | E4BP4 | NFIL3, NF-IL3A | [2]-EKRR-LNDLVLENKLIALGEENATLKAELLSLKLKFGLIS- | 194 |
| | | | STAYAQEIQKLSNSTAVYFQDYQTSKSNV-[5] | |
| E4BP4 | AC012616 | | LNDAAIAGRLAALMEENALLKGELKALKLRFGLLPLT | 195 |
| FOS | Fos | GOS7 | [2]-FRRERNKMAAAKCRNRRR- | 196 |
| | | | ELTDTLQAETDQLEDEKSALQTEIANLLKEKEKLEFILAAHR- | |
| | | | PACKIPDDLGFPEEMS-[5] | |
| FOS | FosB | GOS3 | [2]-NRERNKLAAAKCRNRRR- | 197 |
| | | | ELTDRLQAETDQLEEEKAELESEIAELQKEKERLEFVLVAHK-PGCKIPYEA-[5] | |
| FOS | Fra1 | FOSL1 | [2]-NRRK-ELTDFLQAETDKLEDEKSGLQREIEELQKQKERLELVLEAHR- | 198 |
| | | | PICKIPEGAKEGDTGSTS-[5] | |
| FOS | Fra2 | FOSL2 | [2]-ERNKLAAAKCRNRRR- | 199 |
| | | | ELTEKLQAETEELEEEKSGLQKEIAELQKEKEKLEFMLVAHG-PVCKISP-[5] | |
| Jun | cJun | AP-1 | [2]-QRKL-ERIARLEEKVKTLKAQNSELASTANMLREQVAQLKQKVMNH- | 200 |
| | | | VNSGCQLMLTQQY-[5] | |
| Jun | JunB | — | [2]-NIKVERKRLRNRLAATKCRKRKL- | 201 |
| | | | ERIARLEDKVKTLKAENAGLSSTAGLLREQVAQLKQKVMTH- | |
| | | | VSNGCQLLLGVKGHAFY-[5] | |
| Jun | JunD | — | [1]-DMDTQERIKAERKRLRNRIAASKCRKRKL- | 202 |
| | | | ERISRLEEKVKTLKSQNTELASTASLLREQVAQLKQKVLSHV-N-[5] | |
| IgMAF | cMaf | RIPE3b1 | [2]-QRV-QQRHVLESEKNQLLQQVDHLKQEISRLVRERDAYKEKYEKLVSSGFREN- | 203 |
| | | | GSSSDNPSSPEFFM-[] | |
| IgMAF | MafB | — | [2]-QYKRV-QQKHHLENEKTQLIQQVEQLKQEVSRLARERDAYKVKCEKLANSG- | 204 |
| | | | FREAGSTSDSPSSPEFFL-[] | |
| IgMAF | MafA | — | QQRHILESEKCQLQSQVEQLKLEVGRLAKERDLYKEKYEKLAGRGG | 205 |
| IgMAF | NRL | — | QQRRGLEAERARLAAQLDALRAEVARLARERDLYKARCDRLTSSG | 206 |
| OASIS | OASIS | LZIP, LZIP-alpha, luman | [1]-[]-EYVECLEKKVETFTSENNELWKKVETLENANRTLLQQLQKLQTLVTNKISR-[]- | 207 |
| | | | [5] | |
| OASIS | CREB-H | AIBZIP | [1]-[]-EYIDGLETRMSACTAQNQELQRKVLHLEKQNLSLLEQLKKLQAIVVQSTS-[]- | 208 |
| | | | [5] | |
| OASIS | CREB3 | — | [2]-WRRKIRNKRSAQESRRKKK- | 209 |
| | | | VYVGGLESRVLKYTAQNMELQNKVQLLEEQNLSLLDQLRKLQAMVIEISNKTSS-[]- | |
| | | | [8] | |
| OASIS | CREB4 | — | [2]-WRKIRNKQSAQDSRRRKK- | 210 |
| | | | EYIDGLESRVAACSAQNQELQKKVQELERHNISLVAQLRQLQTLIAQTSNKAAQTST- | |
| | | | CVL-[5] | |
| OASIS | AC022173 | — | EYMDSLEDDVESCSTENLELRKKVEVLENTNRASHGELV | 211 |
| PAR | DBP | TAXREB302 | [1]-EEQKDEKYWSRRYKNNEAAKRSRDARR- | 212 |
| | | | LKENQISVRAAFLEKENALLRQEVVAVRQELSHYRAVLSRYQAQ-HGAL-[] | |
| PAR | HLF | — | [3]-[]-LKENQIAIRASFLEKENSALRQEVADLRKELGKCKNILAKYEARH-[]-[5] | 213 |
| PAR | TEF | TEF | [2]-NWTRRKKNNVAAKRSRDARR- | 214 |
| | | | LKENQITIRAAFLEKENTALRTEVAELRKEVGKCKTIVSKYETK-YGPL-[] | |
| smMAF | MafG | hMaf | [2]-GV-TQKEELEKQKAELQQEVEKLASENASMKLELDALRSKYEALQTFARTVARS- | 215 |
| | | | PVAPARGPLAAGLGPLVPGKVAATSVITIVKSKTDA-[5] | |
| smMAF | MafK | NF-E2 p18 subunit | [1]-[]-TQKEELERQRVELQQEVEKLARENSSMRLELDALRSKYEALQTFARTVAR-[]- | 216 |
| | | | [5] | |
| smMAF | MafF | — | CQKEELQKQKSELEREVDKLARENAAMRLELDALRGKCEALQGFARSVAAARG | 217 |
| XBP | XBP-1 | TREB5, TREB | [2]-QTARDRKK-ARMSELEQQVVDLEEENQKLLLENQLLREKTHGLVVENQEL- | 218 |
| | | | RQRLGMDALVAEEEAEAKGNEV-[5] | |
| YEAST^{e} | ARR1 | YAP8, ACRI, YPR199C | [2]-PSQNAFRKRKL-ERLEELEKKEAQLTVTNDQIHILKKENELLHF- | 219 |
| | | | MLRSLLTERNMP-[14] | |
| YEAST^{e} | CIN5 | YAP4, YOR028C | [2]-KRAAQNRSAQKAFRQRRE- | 220 |
| | | | KYIKNLEEKSKLFDGLMKENSELKKMIESLKSKLKE-[]-[] | |
| YEAST^{e} | YAP1 | PDR4,SNQ3,PAR1, | [2]-EKRTAQNRAAQRAFRERKE- | 221 |
| | | YM9571.12, YML007W | RKMKELEKKVQSLESIQQQNEVEATFLRDQLITLD-[]-[15] | |
| YEAST^{e} | YAP2 | CAD1, YDR423C | [4]-EDTPPNPKKEIEFPGGSPGRKRIDSEAKSRRTAQNRAAQRAFRDRKE- | 222 |
| | | | AKMKSLQERVELLEQK- | |
| | | | DAQNKTTTDFLLCSLKSLLSEITKYRAKNSDDERILAFLDDL-[16] | |
| YEAST^{e} | YAP3 | YHL009C | [4]-EDTPPNPKKEIEFQAKKKAQNRAAQKAFRERKE- | 223 |
| | | | ARMKELQDKLLESERNRQSLLKEIEELRKANTEINAENRLLLRSGN-[]-[17] | |
| YEAST^{e} | YAP5 | YIR018W | [4]-EDTPPNPKKEIEFPKRQNRDAQRAYRERKN- | 224 |
| | | | NKLQVLEETIESLSKVVKNYETKLNRLQNELQAKESENHALKQKLETLTLKQASV- | |
| | | | PAQDPILON-[18] | |
| YEAST^{e} | YAP7 | YOL028C | [2]-QQNRDAQRAYRERRT- | 225 |
| | | | TRIQVLEEKVEMLHNLVDDWQRKYKLLESEFSDTKENLQKSIALNNELQKAL- | |
| | | | PLIVNTPFQQQPEN-[15] | |
| YEAST^{e} | MET28 | YIR017C | [4]-EDTPPNPKKEIEFGIRKK- | 226 |
| | | | QKNFENMNKLQNLNTQINKLRDRIEQLNKENEFWKAKLNDINEIKS- | |
| | | | LKLLNDIKRRNMG-[19] | |
| YEAST^{e} | GCN4 | ARG9, AAS3, YEL009C | [4]-EDTPPNPKKEIEFHPAALKRARNTEAARRSRARKL- | 227 |
| | | | QRMKQLEDKVEELLSKNYHLENEVARLKKLVGDAAR-[]-[20] | |
| YEAST^{e} | MET4 | YNL103W | [4]-EDTPPNPKKEIEFDDDEDLLIQSKKSHQKKK- | 228 |
| | | | LKEKELESSIHELTEIAASLQKRIHTLETENKLLKNLVLSSGET-[]-[18] | |

PAIRCOIL probability drops below 10%. The register of the coiled-coil domain is shown at the bottom of the column starting at the f position. Sequences were aligned according to sequence similarity of the coiled-coil domain using CLUSTALX. Additional protein sequences denoted by the numbers inside the square brackets are as follows: [1]through [4]-MSYYHHHHHLESTSLYKKA, followed by GSGS [1], GSEF [2], GSGR [3] and GFDD [4]; [5]-LE; [6]-KVE; [7]-QLE; [8]-RLE; [9]-E; [10]-FLE; [11]-YLEII; [12]-ALE; [13]-NLE; [14]-CSSNTQLSCTKWLIRGC; [15]-SSNTQLSCTKWLIRGC; [16]-HLE; [17]-STCRDL; [18]-FSTCRDL; [19]-LDLQRSMNRRY; [20]-PAEIYES. The initial methionine in sequences [1]through [4] is cleaved in vivo during expression.

In some embodiments, the at least two leucine zipper motifs may be operatively linked to each other via any of various linkers. In some embodiments, the linker comprises the amino acid sequence of SEQ ID NO: 11, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 11. In some embodiments, the nucleotide sequence that encodes the linker comprises the nucleotide sequence that encodes the amino acid sequence of SEQ ID NO: 11, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 11. In some embodiments, the nucleotide sequence that encodes the linker comprises the nucleotide sequence set forth in SEQ ID NO: 12, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 12. In some embodiments, the nucleotide sequence that encodes the linker comprises the nucleotide sequence set forth in SEQ ID NO: 13, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 13. In some embodiments, the linker comprises the amino acid sequence set forth in SEQ ID NO: 11. In some embodiments, the nucleotide sequence that encodes linker comprises the nucleotide sequence set forth in SEQ ID NO: 12. In some embodiments, the nucleotide sequence that encodes linker comprises the nucleotide sequence set forth in SEQ ID NO: 13.

In some embodiments, the first extracellular region of the chimeric cytokine receptor comprises the amino acid sequence of SEQ ID NO: 158, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 158. In some embodiments, the nucleotide sequence that encodes the first extracellular region of the chimeric cytokine receptor comprises the nucleotide sequence that encodes the amino acid sequence of SEQ ID NO: 158, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 158. In some embodiments, the nucleotide sequence that encodes the first extracellular region of the chimeric cytokine receptor comprises the nucleotide sequence set forth in SEQ ID NO: 159, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 159. In some embodiments, the first extracellular region of the chimeric cytokine receptor comprises the amino acid sequence set forth in SEQ ID NO: 158. In some embodiments, the nucleotide sequence that encodes the first polypeptide of the chimeric cytokine receptor comprises the nucleotide sequence set forth in SEQ ID NO: 159.

In some embodiment, the second extracellular region of the chimeric cytokine receptor comprises the amino acid sequence of SEQ ID NO: 160, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 160. In some embodiments, the nucleotide sequence that encodes the second extracellular region of the chimeric cytokine receptor comprises the nucleotide sequence that encodes the amino acid sequence of SEQ ID NO: 160, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 160. In some embodiments, the nucleotide sequence that encodes the second extracellular region of the chimeric cytokine receptor comprises the nucleotide sequence set forth in SEQ ID NO: 161, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 161. In some embodiments, the second extracellular region of the chimeric cytokine receptor comprises the amino acid sequence set forth in SEQ ID NO: 160. In certain embodiments, the nucleotide sequence that encodes the second polypeptide of the chimeric cytokine receptor comprises the nucleotide sequence set forth in SEQ ID NO: 161.

### Hinge Region

In some embodiments, the chimeric cytokine receptor disclosed herein may comprise a first polypeptide optionally comprising a first hinge region. In some embodiments, the chimeric cytokine receptor disclosed herein may comprise a second polypeptide optionally comprising a second hinge region.

The first and/or second hinge region may be derived from all or part of naturally occurring molecules, such as from all or part of the extracellular region of CD8, CD4, or CD28, or from all or part of an antibody constant region. Alternatively, the first and/or second hinge region may be a synthetic sequence that corresponds to a naturally occurring hinge region sequence or may be an entirely synthetic hinge region sequence.

Non-limiting examples of hinge regions which may be used in accordance with the invention include a part of human CD8a, partial extracellular region of CD28, FcγRllla receptor, IgG, IgM, IgA, IgD, IgE, an Ig hinge, or functional fragment thereof. The hinge may be mutated to prevent Fc receptor binding. The hinge region can be derived from CD8a stalk, CD28, or IgG1. In certain embodiments, the hinge region is derived from CD8a stalk. In various embodiments, the hinge region is derived from CD28. The hinge region can provide flexibility and accessibility between the extracellular region and the transmembrane region.

In some embodiments, the first and/or second hinge may be derived, for example, from IgG1, IgG2, IgG3, IgG4, CD28, or CD8a.

In some embodiments, the first and/or second hinge region may be derived from one or more cytokine receptor chains. As a non-limiting example, the first and/or second hinge regions may be derived from cytokine receptor chain(s) of the IL-2 receptor, IL-7 receptor, IL-15 receptor, IL-21 receptor, IL-12 receptor, IL-9 receptor, IL-4 receptor, IL-23 receptor, IL-10 receptor, or IL-27 receptor.

In some embodiments, the first hinge region may be derived from the same first cytokine receptor chain as the first intracellular signaling region. In some embodiments, the second hinge region may be derived from the same second cytokine receptor chain as the second intracellular signaling region.

In some embodiments, the first and/or second hinge regions are derived from cytokine receptor chain(s) of a gamma cytokine receptor. In some embodiments, the first and/or the second hinge regions may be derived from the common gamma chain (γc).

In some embodiments, the first and/or second hinge regions may be derived from a molecule different from the first and/or second cytokine receptor chain(s) from which the first and/or second intracellular signaling region(s) may be derived.

In some embodiments, the first and second hinge regions are the same. In some embodiments, the first and second hinge regions are different.

The hinge region may comprise up to 300 amino acids, from 10 to 100 amino acids, or from 25 to 50 amino acids.

In some embodiments, the hinge region derived from the common gamma chain (γc) comprises the amino acid sequence of SEQ ID NO: 30, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 30. In some embodiments, the nucleotide sequence that encodes the hinge region derived from the common gamma chain (γc) comprises the nucleotide sequence that encodes the amino acid sequence of SEQ ID NO: 30, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 30. In some embodiments, the nucleotide sequence that encodes the hinge region derived from the common gamma chain (γc) comprises the nucleotide sequence set forth in SEQ ID NO: 31, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 31. In some embodiments, the hinge region derived from the common gamma chain (γc) comprises the amino acid sequence set forth in SEQ ID NO: 30. In some embodiments, the nucleotide sequence that encodes hinge region derived from the common gamma chain (γc) comprises the nucleotide sequence set forth in SEQ ID NO: 31.

In some embodiments, the hinge region derived from the IL-2 receptor beta chain comprises the amino acid sequence of SEQ ID NO: 14, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 14. In some embodiments, the nucleotide sequence that encodes the hinge region derived from the IL-2 receptor beta chain comprises the nucleotide sequence that encodes the amino acid sequence of SEQ ID NO: 14, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 14. In some embodiments, the nucleotide sequence that encodes the hinge region derived from the IL-2 receptor beta chain comprises the nucleotide sequence set forth in SEQ ID NO: 15, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 15. In some embodiments, the hinge region derived from the IL-2 receptor beta chain comprises the amino acid sequence set forth in SEQ ID NO: 14. In some embodiments, the nucleotide sequence that encodes hinge region derived from the IL-2 receptor beta chain comprises the nucleotide sequence set forth in SEQ ID NO: 15.

In some embodiments, the hinge region derived from the IL-7 receptor alpha chain comprises the amino acid sequence of SEQ ID NO: 44, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 44. In some embodiments, the nucleotide sequence that encodes the hinge region derived from the IL-7 receptor alpha chain comprises the nucleotide sequence that encodes the amino acid sequence of SEQ ID NO: 44, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 44. In some embodiments, the nucleotide sequence that encodes the hinge region derived from the IL-7 receptor alpha chain comprises the nucleotide sequence set forth in SEQ ID NO: 45, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 45. In some embodiments, the hinge region derived from the IL-7 receptor alpha chain comprises the amino acid sequence set forth in SEQ ID NO: 44. In some embodiments, the nucleotide sequence that encodes hinge region derived from the IL-7 receptor alpha chain comprises the nucleotide sequence set forth in SEQ ID NO: 45.

In some embodiments, the hinge region derived from the IL-21 receptor chain comprises the amino acid sequence of SEQ ID NO: 54 or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 54. In some embodiments, the nucleotide sequence that encodes the hinge region derived from the IL-21 receptor chain comprises the nucleotide sequence that encodes the amino acid sequence of SEQ ID NO: 54, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 54. In some embodiments, the nucleotide sequence that encodes the hinge region derived from the IL-21 receptor chain comprises the nucleotide sequence set forth in SEQ ID NO: 55, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 55. In some embodiments, the hinge region derived from the IL-21 receptor chain comprises the amino acid sequence set forth in SEQ ID NO: 54. In some embodiments, the nucleotide sequence that encodes hinge region derived from the IL-21 receptor chain comprises the nucleotide sequence set forth in SEQ ID NO: 55.

In some embodiments, the hinge region derived from the IL-9 receptor chain comprises the amino acid sequence of SEQ ID NO: 64 or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 64. In some embodiments, the nucleotide sequence that encodes the hinge region derived from the IL-9 receptor chain comprises the nucleotide sequence that encodes the amino acid sequence of SEQ ID NO: 64, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 64. In some embodiments, the nucleotide sequence that encodes the hinge region derived from the IL-9 receptor chain comprises the nucleotide sequence set forth in SEQ ID NO: 65, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 65. In some embodiments, the hinge region derived from the IL-9 receptor chain comprises the amino acid sequence set forth in SEQ ID NO: 64. In some embodiments, the nucleotide sequence that encodes hinge region derived from the IL-9 receptor chain comprises the nucleotide sequence set forth in SEQ ID NO: 65.

In some embodiments, the hinge region derived from the IL-4 receptor alpha chain comprises the amino acid sequence of SEQ ID NO: 74 or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 74. In some embodiments, the nucleotide sequence that encodes the hinge region derived from the IL-4 receptor alpha chain comprises the nucleotide sequence that encodes the amino acid sequence of SEQ ID NO: 74, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 74. In some embodiments, the nucleotide sequence that encodes the hinge region derived from the IL-4 receptor alpha chain comprises the nucleotide sequence set forth in SEQ ID NO: 75, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 75. In some embodiments, the hinge region derived from the IL-4 receptor alpha chain comprises the amino acid sequence set forth in SEQ ID NO: 74. In some embodiments, the nucleotide sequence that encodes hinge region derived from the IL-4 receptor alpha chain comprises the nucleotide sequence set forth in SEQ ID NO: 75.

In some embodiments, the hinge region derived from the IL-12 receptor beta 1 chain comprises the amino acid sequence of SEQ ID NO: 84 or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 84. In some embodiments, the nucleotide sequence that encodes the hinge region derived from the IL-12 receptor beta 1 chain comprises the nucleotide sequence that encodes the amino acid sequence of SEQ ID NO: 84, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 84. In some embodiments, the nucleotide sequence that encodes the hinge region derived from the IL-12 receptor beta 1 chain comprises the nucleotide sequence set forth in SEQ ID NO: 85, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 85. In some embodiments, the hinge region derived from the IL-12 receptor beta 1 chain comprises the amino acid sequence set forth in SEQ ID NO: 84. In some embodiments, the nucleotide sequence that encodes hinge region derived from the IL-12 receptor beta 1 chain comprises the nucleotide sequence set forth in SEQ ID NO: 85.

In some embodiments, the hinge region derived from the IL-12 receptor beta 2 chain comprises the amino acid sequence of SEQ ID NO: 94 or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 94. In some embodiments, the nucleotide sequence that encodes the hinge region derived from the IL-12 receptor beta 2 chain comprises the nucleotide sequence that encodes the amino acid sequence of SEQ ID NO: 94, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 94. In some embodiments, the nucleotide sequence that encodes the hinge region derived from the IL-12 receptor beta 2 chain comprises the nucleotide sequence set forth in SEQ ID NO: 95, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 95. In some embodiments, the hinge region derived from the IL-12 receptor beta 2 chain comprises the amino acid sequence set forth in SEQ ID NO: 94. In some embodiments, the nucleotide sequence that encodes hinge region derived from the IL-12 receptor beta 2 chain comprises the nucleotide sequence set forth in SEQ ID NO: 95.

In some embodiments, the hinge region derived from the IL-23 receptor chain comprises the amino acid sequence of SEQ ID NO: 104 or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 104. In some embodiments, the nucleotide sequence that encodes the hinge region derived from the IL-23 receptor chain comprises the nucleotide sequence that encodes the amino acid sequence of SEQ ID NO: 104, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 104. In some embodiments, the nucleotide sequence that encodes the hinge region derived from the IL-23 receptor chain comprises the nucleotide sequence set forth in SEQ ID NO: 105, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 105. In some embodiments, the hinge region derived from the TL-23 receptor chain comprises the amino acid sequence set forth in SEQ ID NO: 104. In some embodiments, the nucleotide sequence that encodes hinge region derived from the TL-23 receptor chain comprises the nucleotide sequence set forth in SEQ ID NO: 105.

In some embodiments, the hinge region derived from the IL-10 receptor alpha chain comprises the amino acid sequence of SEQ ID NO: 114 or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 114. In some embodiments, the nucleotide sequence that encodes the hinge region derived from the IL-10 receptor alpha chain comprises the nucleotide sequence that encodes the amino acid sequence of SEQ ID NO: 114, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 114. In some embodiments, the nucleotide sequence that encodes the hinge region derived from the IL-10 receptor alpha chain comprises the nucleotide sequence set forth in SEQ ID NO: 115, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 115. In some embodiments, the hinge region derived from the IL-10 receptor alpha chain comprises the amino acid sequence set forth in SEQ ID NO: 114. In some embodiments, the nucleotide sequence that encodes hinge region derived from the IL-10 receptor alpha chain comprises the nucleotide sequence set forth in SEQ ID NO: 115.

In some embodiments, the hinge region derived from the IL-10 receptor beta chain comprises the amino acid sequence of SEQ ID NO: 124 or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 124. In some embodiments, the nucleotide sequence that encodes the hinge region derived from the IL-10 receptor beta chain comprises the nucleotide sequence that encodes the amino acid sequence of SEQ ID NO: 124, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 124. In some embodiments, the nucleotide sequence that encodes the hinge region derived from the IL-10 receptor beta chain comprises the nucleotide sequence set forth in SEQ ID NO: 125, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 125. In some embodiments, the hinge region derived from the IL-10 receptor beta chain comprises the amino acid sequence set forth in SEQ ID NO: 124. In some embodiments, the nucleotide sequence that encodes hinge region derived from the IL-10 receptor beta chain comprises the nucleotide sequence set forth in SEQ ID NO: 125.

In some embodiments, the hinge region derived from the glycoprotein 130 (gp130 or IL-6 beta) chain comprises the amino acid sequence of SEQ ID NO: 134 or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 134. In some embodiments, the nucleotide sequence that encodes the hinge region derived from the glycoprotein 130 (gp130 or IL-6 beta) chain comprises the nucleotide sequence that encodes the amino acid sequence of SEQ ID NO: 134, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 134. In some embodiments, the nucleotide sequence that encodes the hinge region derived from the glycoprotein 130 (gp130 or IL-6 beta) chain comprises the nucleotide sequence set forth in SEQ ID NO: 135, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 135. In some embodiments, the hinge region derived from the glycoprotein 130 (gp130 or IL-6 beta) chain comprises the amino acid sequence set forth in SEQ ID NO: 134. In some embodiments, the nucleotide sequence that encodes hinge region derived from the glycoprotein 130 (gp130 or IL-6 beta) chain comprises the nucleotide sequence set forth in SEQ ID NO: 135.

### Leader Sequence

In various embodiments, the extracellular region comprises a leader sequence. The leader sequence may be positioned at the N-terminus of the extracellular region. The leader sequence may be optionally cleaved from the extracellular region during cellular processing and localization of the chimeric cytokine receptor to the cellular membrane. Any of various leader sequences known to one of skill in the art may be used as the leader sequence. Non-limiting examples of peptides from which the leader sequence may be derived include FcR, human immunoglobulin heavy chain variable region, CD8a, or any of various other proteins secreted by T cells. In various embodiments, the leader sequence is compatible with the secretory pathway of a T cell. In certain embodiments, the leader sequence is derived from human immunoglobulin heavy chain.

In some embodiments, the first and/or the second polypeptide of the chimeric cytokine receptor disclosed herein may comprise a leader sequence. In some embodiments, the leader sequence may be derived from an immunoglobulin heavy chain variable region or colony stimulating factor 2 receptor alpha chain (CSF2RA). In some embodiments, the leader sequence may be derived from an immunoglobulin heavy chain variable region.

In some embodiments, the leader sequence comprises the amino acid sequence of SEQ ID NO: 3, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 3. In some embodiments, the nucleotide sequence that encodes the leader sequence comprises the nucleotide sequence that encodes the amino acid sequence of SEQ ID NO: 3, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 3. In some embodiments, the nucleotide sequence that encodes the leader sequence comprises the nucleotide sequence set forth in SEQ ID NO: 4, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 4. In some embodiments, the nucleotide sequence that encodes the leader sequence comprises the nucleotide sequence set forth in SEQ ID NO: 5, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 5. In some embodiments, the leader sequence comprises the amino acid sequence set forth in SEQ ID NO: 3. In some embodiments, the nucleotide sequence that encodes leader sequence comprises the nucleotide sequence set forth in SEQ ID NO: 4. In some embodiments, the nucleotide sequence that encodes leader sequence comprises the nucleotide sequence set forth in SEQ ID NO: 5.

In some embodiments, the first and the second leader sequences are the same.

In some embodiments, the first and the second leader sequences are different.

### Transmembrane Region

In some embodiments, the chimeric cytokine receptor disclosed herein may comprise a first polypeptide comprising a first transmembrane region. In some embodiments, the chimeric cytokine receptor disclosed herein may comprise a second polypeptide comprising a second transmembrane region.

In some embodiments of any of the chimeric cytokine receptors disclosed herein, the first transmembrane region may be derived from the same cytokine receptor chain as the first intracellular signaling region.

In some embodiments of any of the chimeric cytokine receptors disclosed herein, the second transmembrane region may be derived from the same cytokine receptor chain as the second intracellular signaling region.

In some embodiments, the first and/or second transmembrane regions may be derived from cytokine receptor chain(s) of the IL-2 receptor, IL-7 receptor, IL-15 receptor, IL-21 receptor, IL-12 receptor, IL-9 receptor, TL-4 receptor, IL-23 receptor, IL-10 receptor, or IL-27 receptor.

In some embodiments, the first and/or second transmembrane regions may be derived from a gamma cytokine receptor. In some embodiments, the first and/or the second transmembrane region may be derived from the common gamma chain (γc).

In some embodiments, the first and/or second transmembrane regions may be derived from a molecule different from the first and/or second cytokine receptor chain(s) from which the first and/or second intracellular signaling region(s) may be derived.

In some embodiments, the first and second transmembrane regions are the same. In some embodiments, the first and second transmembrane regions are different.

In some embodiments, the first and/or second transmembrane region derived from the common gamma chain (γc) comprises the amino acid sequence of SEQ ID NO: 32, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 32. In some embodiments, the nucleotide sequence that encodes the first and/or second transmembrane region derived from the common gamma chain (γc) comprises the nucleotide sequence that encodes the amino acid sequence of SEQ ID NO: 32, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 32. In some embodiments, the nucleotide sequence that encodes the first and/or second transmembrane region derived from the common gamma chain (γc) comprises the nucleotide sequence set forth in SEQ ID NO: 33, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 33. In some embodiments, the first and/or second transmembrane region derived from the common gamma chain (γc) comprises the amino acid sequence set forth in SEQ ID NO: 32. In some embodiments, the nucleotide sequence that encodes the first and/or second transmembrane region derived from the common gamma chain (γc) comprises the nucleotide sequence set forth in SEQ ID NO: 33.

In some embodiments, the first and/or second transmembrane region derived from the IL-2 receptor beta chain comprises the amino acid sequence of SEQ ID NO: 16, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 16. In certain embodiments, the nucleotide sequence that encodes the first and/or second transmembrane region derived from the IL-2 receptor beta chain comprises the nucleotide sequence that encodes the amino acid sequence of SEQ ID NO: 16, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 16. In certain embodiments, the nucleotide sequence that encodes the first and/or second transmembrane region derived from the IL-2 receptor beta chain comprises the nucleotide sequence set forth in SEQ ID NO: 17, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 17. In certain embodiments, the first and/or second transmembrane region derived from the IL-2 receptor beta chain comprises the amino acid sequence set forth in SEQ ID NO: 16. In certain embodiments, the nucleotide sequence that encodes the first and/or second transmembrane region derived from the IL-2 receptor beta chain comprises the nucleotide sequence set forth in SEQ ID NO: 17.

In some embodiments, the first and/or second transmembrane region derived from the IL-7 receptor alpha chain comprises the amino acid sequence of SEQ ID NO: 46, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 46. In certain embodiments, the nucleotide sequence that encodes the first and/or second transmembrane region derived from the IL-7 receptor alpha chain comprises the nucleotide sequence that encodes the amino acid sequence of SEQ ID NO: 46, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 46. In certain embodiments, the nucleotide sequence that encodes the first and/or second transmembrane region derived from the IL-7 receptor alpha chain comprises the nucleotide sequence set forth in SEQ ID NO: 47, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 47. In certain embodiments, the first and/or second transmembrane region derived from the IL-7 receptor alpha chain comprises the amino acid sequence set forth in SEQ ID NO: 46. In certain embodiments, the nucleotide sequence that encodes the first and/or second transmembrane region derived from the IL-7 receptor alpha chain comprises s the nucleotide sequence set forth in SEQ ID NO: 47..

In some embodiments, the first and/or second transmembrane region derived from the IL-21 receptor chain comprises the amino acid sequence of SEQ ID NO: 56, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 56. In certain embodiments, the nucleotide sequence that encodes the first and/or second transmembrane region derived from the IL-21 receptor chain comprises the nucleotide sequence that encodes the amino acid sequence of SEQ ID NO: 56, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 56. In certain embodiments, the nucleotide sequence that encodes the first and/or second transmembrane region derived from the IL-21 receptor chain comprises the nucleotide sequence set forth in SEQ ID NO: 57, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 57. In certain embodiments, the first and/or second transmembrane region derived from the IL-21 receptor chain comprises the amino acid sequence set forth in SEQ ID NO: 56. In certain embodiments, the nucleotide sequence that encodes the first and/or second transmembrane region derived from the IL-21 receptor chain comprises the nucleotide sequence set forth in SEQ ID NO: 57.

In some embodiments, the first and/or second transmembrane region derived from the TL-9 receptor chain comprises the amino acid sequence of SEQ ID NO: 66, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 66. In certain embodiments, the nucleotide sequence that encodes the first and/or second transmembrane region derived from the TL-9 receptor chain comprises the nucleotide sequence that encodes the amino acid sequence of SEQ ID NO: 66, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 66. In certain embodiments, the nucleotide sequence that encodes the first and/or second transmembrane region derived from the IL-9 receptor chain comprises the nucleotide sequence set forth in SEQ ID NO: 67, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 67. In certain embodiments, the first and/or second transmembrane region derived from the IL-9 receptor chain comprises the amino acid sequence set forth in SEQ ID NO: 66. In certain embodiments, the nucleotide sequence that encodes the first and/or second transmembrane region derived from the IL-9 receptor chain comprises the nucleotide sequence set forth in SEQ ID NO: 67.

In some embodiments, the first and/or second transmembrane region derived from the IL-4 receptor alpha chain comprises the amino acid sequence of SEQ ID NO: 76, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 76. In certain embodiments, the nucleotide sequence that encodes the first and/or second transmembrane region derived from the IL-4 receptor alpha chain comprises the nucleotide sequence that encodes the amino acid sequence of SEQ ID NO: 76, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 76. In certain embodiments, the nucleotide sequence that encodes the first and/or second transmembrane region derived from the IL-4 receptor alpha chain comprises the nucleotide sequence set forth in SEQ ID NO: 77, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 77. In certain embodiments, the first and/or second transmembrane region derived from the IL-4 receptor alpha chain comprises the amino acid sequence set forth in SEQ ID NO: 76. In certain embodiments, the nucleotide sequence that encodes the first and/or second transmembrane region derived from the IL-4 receptor alpha chain comprises the nucleotide sequence set forth in SEQ ID NO: 77.

In some embodiments, the first and/or second transmembrane region derived from the IL-12 receptor beta 1 chain comprises the amino acid sequence of SEQ ID NO: 86, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 86. In some embodiments, the nucleotide sequence that encodes the first and/or second transmembrane region derived from the IL-12 receptor beta 1 chain comprises the nucleotide sequence that encodes the amino acid sequence of SEQ ID NO: 86, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 86. In some embodiments, the nucleotide sequence that encodes the first and/or second transmembrane region derived from the IL-12 receptor beta 1 chain comprises the nucleotide sequence set forth in SEQ ID NO: 87, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 87. In some embodiments, the first and/or second transmembrane region derived from the IL-12 receptor beta 1 chain comprises the amino acid sequence set forth in SEQ ID NO: 86. In some embodiments, the nucleotide sequence that encodes the first transmembrane region derived from the IL-12 receptor beta 1 chain comprises the nucleotide sequence set forth in SEQ ID NO: 87.

In some embodiments, the first and/or second transmembrane region derived from the IL-12 receptor beta 2 chain comprises the amino acid sequence of SEQ ID NO: 96, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 96. In certain embodiments, the nucleotide sequence that encodes the first and/or second transmembrane region derived from the IL-12 receptor beta 2 chain comprises the nucleotide sequence that encodes the amino acid sequence of SEQ ID NO: 96, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 96. In certain embodiments, the nucleotide sequence that encodes the first and/or second transmembrane region derived from the IL-12 receptor beta 2 chain comprises the nucleotide sequence set forth in SEQ ID NO: 97, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 97. In certain embodiments, the first and/or second transmembrane region derived from the IL-12 receptor beta 2 chain comprises the amino acid sequence set forth in SEQ ID NO: 96. In certain embodiments, the nucleotide sequence that encodes the first and/or second transmembrane region derived from the IL-12 receptor beta 2 chain comprises the nucleotide sequence set forth in SEQ ID NO: 97.

In some embodiments, the first and/or second transmembrane region derived from the TL-23 receptor chain comprises the amino acid sequence of SEQ ID NO: 106, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 106. In some embodiments, the nucleotide sequence that encodes the first and/or second transmembrane region derived from the TL-23 receptor chain comprises the nucleotide sequence that encodes the amino acid sequence of SEQ ID NO: 106, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 106. In some embodiments, the nucleotide sequence that encodes the first and/or second transmembrane region derived from the IL-23 receptor chain comprises the nucleotide sequence set forth in SEQ ID NO: 107, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 107. In some embodiments, the first and/or second transmembrane region derived from the IL-23 receptor chain comprises the amino acid sequence set forth in SEQ ID NO: 106. In some embodiments, the nucleotide sequence that encodes the first and/or second transmembrane region derived from the IL-23 receptor chain comprises the nucleotide sequence set forth in SEQ ID NO: 107.

In some embodiments, the first and/or second transmembrane region derived from the IL-10 receptor alpha chain comprises the amino acid sequence of SEQ ID NO: 116, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 116. In some embodiments, the nucleotide sequence that encodes the first and/or second transmembrane region derived from the IL-10 receptor alpha chain comprises the nucleotide sequence that encodes the amino acid sequence of SEQ ID NO: 116, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 116. In some embodiments, the nucleotide sequence that encodes the first and/or second transmembrane region derived from the IL-10 receptor alpha chain comprises the nucleotide sequence set forth in SEQ ID NO: 117, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 117. In some embodiments, the first and/or second transmembrane region derived from the IL-10 receptor alpha chain comprises the amino acid sequence set forth in SEQ ID NO: 116. In some embodiments, the nucleotide sequence that encodes the first and/or second transmembrane region derived from the IL-10 receptor alpha chain comprises the nucleotide sequence set forth in SEQ ID NO: 117.

In some embodiments, the first and/or second transmembrane region derived from the IL-10 receptor beta chain comprises the amino acid sequence of SEQ ID NO: 126, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 126. In certain embodiments, the nucleotide sequence that encodes the first and/or second transmembrane region derived from the IL-10 receptor beta chain comprises the nucleotide sequence that encodes the amino acid sequence of SEQ ID NO: 126, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 126. In certain embodiments, the nucleotide sequence that encodes the first and/or second transmembrane region derived from the IL-10 receptor beta chain comprises the nucleotide sequence set forth in SEQ ID NO: 127, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 127. In certain embodiments, the first and/or second transmembrane region derived from the IL-10 receptor beta chain comprises the amino acid sequence set forth in SEQ ID NO: 126. In certain embodiments, the nucleotide sequence that encodes the first and/or second transmembrane region derived from the IL-10 receptor beta chain comprises s the nucleotide sequence set forth in SEQ ID NO: 127.

In some embodiments, the first and/or second transmembrane region derived from the glycoprotein 130 (gp130 or IL-6 beta) chain comprises the amino acid sequence of SEQ ID NO: 136, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 136. In certain embodiments, the nucleotide sequence that encodes the first and/or second transmembrane region derived from the glycoprotein 130 (gp130 or IL-6 beta) chain comprises the nucleotide sequence that encodes the amino acid sequence of SEQ ID NO: 136, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 136. In certain embodiments, the nucleotide sequence that encodes the first and/or second transmembrane region derived from the glycoprotein 130 (gp130 or IL-6 beta) chain comprises the nucleotide sequence set forth in SEQ ID NO: 136, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 137. In certain embodiments, the first and/or second transmembrane region derived from the glycoprotein 130 (gp130 or IL-6 beta) chain comprises the amino acid sequence set forth in SEQ ID NO: 137. In certain embodiments, the nucleotide sequence that encodes the first and/or second transmembrane region derived from the glycoprotein 130 (gp130 or IL-6 beta) chain comprises s the nucleotide sequence set forth in SEQ ID NO: 136.

In certain embodiments, the first and/or second transmembrane region is derived from CD8a, CD28, CD8, CD4, CD3ζ, CD40, CD134 (OX-40), or CD7. In a specific embodiment, the transmembrane region is derived from CD8a. In a specific embodiment, the transmembrane region is derived from CD28. The first and/or second transmembrane region may be fused in frame or operably linked between the extracellular region and the intracellular signaling region.

In some instances, the first and/or second transmembrane region can be modified by amino acid substitution, deletions, or insertions to avoid binding of proteins naturally associated with the first and/or second transmembrane region. In certain embodiments, the first and/or second transmembrane region includes additional amino acids to allow for flexibility and/or optimal distance between the regions connected to the transmembrane region.

The first and/or second transmembrane region may be derived from a natural or from a synthetic source. Where the source is natural, the region may be derived from any membrane-bound or transmembrane protein. Non-limiting examples of transmembrane regions of particular use in this disclosure may be derived from (i.e., comprise at least the transmembrane region(s) of) the a, R or (chain of the T-cell receptor, CD28, CD3E, CD3ζ, CD45, CD4, CD5, CD7, CD8, CD8a, CD9, CD16, CD22, CD33, CD37, CD40, CD64, CD80, CD86, CD134 (OX-40), CD137, or CD154. Alternatively, the transmembrane region may be synthetic, in which case the transmembrane region will comprise predominantly hydrophobic residues such as leucine and valine. For example, a triplet of phenylalanine, tryptophan and/or valine can be found at each end of a synthetic transmembrane region.

In some embodiments, the first and/or second transmembrane regions are derived from CD28, CD8, CD4, CD3ζ, CD40, CD134 (OX-40), CD19, or CD7.

In some embodiments, it will be desirable to utilize the transmembrane region of the ζ, η or FcεR1γ chains which contain a cysteine residue capable of disulfide bonding, so that the resulting chimeric protein will be able to form disulfide linked dimers with itself, or with unmodified versions of the ζ, η or FcεR1γ chains or related proteins. In some instances, the transmembrane region will be selected or modified by amino acid substitution to avoid-binding of such regions to the transmembrane regions of the same or different surface membrane proteins to minimize interactions with other members of the receptor complex. In other cases, it will be desirable to employ the transmembrane region of ζ, η or FcεR1γ and -β, MB1 (Igα.), B29 or CD3-γ, ζ, or η, in order to retain physical association with other members of the receptor complex.

### Intracellular Signaling Region

In some embodiments, the chimeric cytokine receptor disclosed herein may comprise a first polypeptide comprising at least a first intracellular signaling region. In some embodiments, the first intracellular signaling region may be derived from a first cytokine receptor chain. In some embodiments, the chimeric cytokine receptor disclosed herein may comprise a second polypeptide comprising at least a second intracellular signaling region. In some embodiments, the second intracellular signaling region may be derived from a first cytokine receptor chain.

In some embodiments, the at least one first and/or second intracellular signaling regions may be derived from cytokine receptor chain(s) of a type I cytokine receptor. Type I cytokine receptors include, but are not limited to, IL-1 receptor, IL-2 receptor, IL-3 receptor, IL-4 receptor, IL-5 receptor, IL-6 receptor, IL-7 receptor, IL-9 receptor, IL-11 receptor, IL-12 receptor, IL-13 receptor, IL-15 receptor, IL-18 receptor, IL-21 receptor, IL-23 receptor, IL-27 receptor, Erythropoietin receptor, G-CSF receptor, Thrombopoietin receptor, GM-CSF receptor, growth hormone receptor, prolactin receptor, leptin receptor, leukemia inhibitory factor receptor, or oncostatin M receptor.

In some embodiments, the at least one first and/or second intracellular signaling regions may be derived from cytokine receptor chain(s) of a type II cytokine receptor. Type II cytokine receptors include, but are not limited to, IFN-α/β receptor, IFN-γ receptor, interferon type III receptor, IL-10 receptor, IL-20 receptor, IL-22 receptor, or IL-28 receptor.

In some embodiments, the at least one first and/or second intracellular signaling regions may be derived from cytokine receptor chain(s) of the IL-2 receptor, IL-7 receptor, IL-15 receptor, IL-21 receptor, IL-12 receptor, IL-9 receptor, IL-4 receptor, IL-23 receptor, IL-10 receptor, or IL-27 receptor, or a combination thereof.

In some embodiments, the at least one first and/or second intracellular signaling regions may be derived from cytokine receptor chain(s) of a gamma cytokine receptor.

In some embodiments, the first or the second intracellular signaling region may be derived from the common gamma chain (γc) (also known as IL-2 receptor gamma chain or IL-2RG, or CD132).

In some embodiments, the first intracellular signaling region may be derived from the common gamma chain (γc), and the second intracellular signaling region is derived from the IL-2 receptor beta chain.

In some embodiments, the first intracellular signaling region may be derived from the common gamma chain (γc), and the second intracellular signaling region is derived from the IL-7 receptor alpha chain.

In some embodiments, the first intracellular signaling region may be derived from the common gamma chain (γc), and the second intracellular signaling region is derived from the IL-21 receptor chain.

In some embodiments, the first intracellular signaling region may be derived from the common gamma chain (γc), and the second intracellular signaling region is derived from the IL-9 receptor chain.

In some embodiments, the first intracellular signaling region may be derived from the common gamma chain (γc), and the second intracellular signaling region is derived from the IL-4 receptor alpha chain.

In some embodiments, the first intracellular signaling region may be derived from the IL-12 receptor beta 1 chain, and the second intracellular signaling region is derived from the IL-12 receptor beta 2 chain.

In some embodiments, the first intracellular signaling region may be derived from the IL-23 receptor chain, and the second intracellular signaling region is derived from the IL-12 receptor beta 2 chain.

In some embodiments, the first intracellular signaling region may be derived from the IL-10 receptor alpha chain, and the second intracellular signaling region is derived from the IL-10 receptor beta chain.

In some embodiments, the first intracellular signaling region may be derived from the IL-27 receptor alpha chain (or WSX-1), and the second intracellular signaling region is derived from glycoprotein 130 (gp130 or IL-6 beta chain).

In some embodiments, the first and/or the second polypeptides comprise two intracellular signaling regions derived from two cytokine receptor chains.

In some embodiments, the first polypeptide comprises an intracellular signaling region derived from the IL-2 receptor beta chain and an intracellular signaling region derived from the IL-21 receptor chain, and the second polypeptide comprises an intracellular signaling region derived from the common gamma chain (γc).

In some embodiments, the first polypeptide comprises an intracellular signaling region derived from the IL-7 receptor alpha chain and an intracellular signaling region derived from the IL-21 receptor chain, and the second polypeptide comprises an intracellular signaling region derived from the common gamma chain (γc).

In some embodiments, the first and/or second intracellular signaling regions may comprise the intracellular signaling region(s) of the cytokine receptor chain(s) from which they are derived. In some embodiments, the first and/or second intracellular signaling regions may comprise a functional fragment of the intracellular signaling region(s) of the cytokine receptor chain(s) from which they are derived.

In some embodiments, the first and/or second intracellular signaling region derived from the common gamma chain (γc) comprises the amino acid sequence of SEQ ID NO: 34, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 34. In some embodiments, the nucleotide sequence that encodes the first and/or second intracellular signaling region derived from the common gamma chain (γc) comprises the nucleotide sequence that encodes the amino acid sequence of SEQ ID NO: 34, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 34. In some embodiments, the nucleotide sequence that encodes the first and/or second intracellular signaling region derived from the common gamma chain (γc) comprises the nucleotide sequence set forth in SEQ ID NO: 35, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 35. In some embodiments, the first and/or second intracellular signaling region derived from the common gamma chain (γc) comprises the amino acid sequence set forth in SEQ ID NO: 34. In some embodiments, the nucleotide sequence that encodes the first and/or second intracellular signaling region derived from the common gamma chain (γc) comprises the nucleotide sequence set forth in SEQ ID NO: 35.

In some embodiments, the first and/or second intracellular signaling region derived from the IL-2 receptor beta chain comprises the amino acid sequence of SEQ ID NO: 18, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 18. In certain embodiments, the nucleotide sequence that encodes the first and/or second intracellular signaling region derived from the IL-2 receptor beta chain comprises the nucleotide sequence that encodes the amino acid sequence of SEQ ID NO: 18, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 18. In certain embodiments, the nucleotide sequence that encodes the first and/or second intracellular signaling region derived from the IL-2 receptor beta chain comprises the nucleotide sequence set forth in SEQ ID NO: 19, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 19. In certain embodiments, the first and/or second intracellular signaling region derived from the IL-2 receptor beta chain comprises the amino acid sequence set forth in SEQ ID NO: 18. In certain embodiments, the nucleotide sequence that encodes the first and/or second intracellular signaling region derived from the IL-2 receptor beta chain comprises the nucleotide sequence set forth in SEQ ID NO: 19.

In some embodiments, the first and/or second intracellular signaling region derived from the IL-7 receptor alpha chain comprises the amino acid sequence of SEQ ID NO: 48, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 48. In certain embodiments, the nucleotide sequence that encodes the first and/or second intracellular signaling region derived from the IL-7 receptor alpha chain comprises the nucleotide sequence that encodes the amino acid sequence of SEQ ID NO: 48, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 48. In certain embodiments, the nucleotide sequence that encodes the first and/or second intracellular signaling region derived from the IL-7 receptor alpha chain comprises the nucleotide sequence set forth in SEQ ID NO: 49, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 49. In certain embodiments, the first and/or second intracellular signaling region derived from the IL-7 receptor alpha chain comprises the amino acid sequence set forth in SEQ ID NO: 48. In certain embodiments, the nucleotide sequence that encodes the first and/or second intracellular signaling region derived from the IL-7 receptor alpha chain comprises the nucleotide sequence set forth in SEQ ID NO: 49.

In some embodiments, the first and/or second intracellular signaling region derived from the IL-21 receptor chain comprises the amino acid sequence of SEQ ID NO: 58, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 58. In certain embodiments, the nucleotide sequence that encodes the first and/or second intracellular signaling region derived from the IL-21 receptor chain comprises the nucleotide sequence that encodes the amino acid sequence of SEQ ID NO: 58, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 58. In certain embodiments, the nucleotide sequence that encodes the first and/or second intracellular signaling region derived from the IL-21 receptor chain comprises the nucleotide sequence set forth in SEQ ID NO: 59, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 59. In certain embodiments, the first and/or second intracellular signaling region derived from the IL-21 receptor chain comprises the amino acid sequence set forth in SEQ ID NO: 58. In certain embodiments, the nucleotide sequence that encodes the first and/or second intracellular signaling region derived from the IL-21 receptor chain comprises the nucleotide sequence set forth in SEQ ID NO: 59.

In some embodiments, the first and/or second intracellular signaling region derived from the IL-9 receptor chain comprises the amino acid sequence of SEQ ID NO: 68, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 68. In certain embodiments, the nucleotide sequence that encodes the first and/or second intracellular signaling region derived from the IL-9 receptor chain comprises the nucleotide sequence that encodes the amino acid sequence of SEQ ID NO: 68, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 68. In certain embodiments, the nucleotide sequence that encodes the first and/or second intracellular signaling region derived from the IL-9 receptor chain comprises the nucleotide sequence set forth in SEQ ID NO: 69, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 69. In certain embodiments, the first and/or second intracellular signaling region derived from the IL-9 receptor chain comprises the amino acid sequence set forth in SEQ ID NO: 68. In certain embodiments, the nucleotide sequence that encodes the first and/or second intracellular signaling region derived from the IL-9 receptor chain comprises the nucleotide sequence set forth in SEQ ID NO: 69.

In some embodiments, the first and/or second intracellular signaling region derived from the IL-4 receptor alpha chain comprises the amino acid sequence of SEQ ID NO: 78, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 78. In certain embodiments, the nucleotide sequence that encodes the first and/or second intracellular signaling region derived from the IL-4 receptor alpha chain comprises the nucleotide sequence that encodes the amino acid sequence of SEQ ID NO: 78, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 78. In certain embodiments, the nucleotide sequence that encodes the first and/or second intracellular signaling region derived from the IL-4 receptor alpha chain comprises the nucleotide sequence set forth in SEQ ID NO: 79, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 79. In certain embodiments, the first and/or second intracellular signaling region derived from the IL-4 receptor alpha chain comprises the amino acid sequence set forth in SEQ ID NO: 78. In certain embodiments, the nucleotide sequence that encodes the first and/or second intracellular signaling region derived from the IL-4 receptor alpha chain comprises the nucleotide sequence set forth in SEQ ID NO: 79.

In some embodiments, the first and/or second intracellular signaling region derived from the IL-12 receptor beta 1 chain comprises the amino acid sequence of SEQ ID NO: 88, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 88. In some embodiments, the nucleotide sequence that encodes the first and/or second intracellular signaling region derived from the IL-12 receptor beta 1 chain comprises the nucleotide sequence that encodes the amino acid sequence of SEQ ID NO: 88, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 88. In some embodiments, the nucleotide sequence that encodes the first and/or second intracellular signaling region derived from the IL-12 receptor beta 1 chain comprises the nucleotide sequence set forth in SEQ ID NO: 89, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 89. In some embodiments, the first and/or second intracellular signaling region derived from the IL-12 receptor beta 1 chain comprises the amino acid sequence set forth in SEQ ID NO: 88. In some embodiments, the nucleotide sequence that encodes the first and/or second intracellular signaling region derived from the IL-12 receptor beta 1 chain comprises the nucleotide sequence set forth in SEQ ID NO: 89.

In some embodiments, the first and/or second intracellular signaling region derived from the IL-12 receptor beta 2 chain comprises the amino acid sequence of SEQ ID NO: 98, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 98. In certain embodiments, the nucleotide sequence that encodes the first and/or second intracellular signaling region derived from the IL-12 receptor beta 2 chain comprises the nucleotide sequence that encodes the amino acid sequence of SEQ ID NO: 98, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 98. In certain embodiments, the nucleotide sequence that encodes the first and/or second intracellular signaling region derived from the IL-12 receptor beta 2 chain comprises the nucleotide sequence set forth in SEQ ID NO: 99, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 99. In certain embodiments, the first and/or second intracellular signaling region derived from the IL-12 receptor beta 2 chain comprises the amino acid sequence set forth in SEQ ID NO: 98. In certain embodiments, the nucleotide sequence that encodes the first and/or second intracellular signaling region derived from the IL-12 receptor beta 2 chain comprises the nucleotide sequence set forth in SEQ ID NO: 99.

In some embodiments, the first and/or second intracellular signaling region derived from the IL-23 receptor chain comprises the amino acid sequence of SEQ ID NO: 108, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 108. In some embodiments, the nucleotide sequence that encodes the first and/or second intracellular signaling region derived from the IL-23 receptor chain comprises the nucleotide sequence that encodes the amino acid sequence of SEQ ID NO: 108, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 108. In some embodiments, the nucleotide sequence that encodes the first and/or second intracellular signaling region derived from the IL-23 receptor chain comprises the nucleotide sequence set forth in SEQ ID NO: 109, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 109. In some embodiments, the first and/or second intracellular signaling region derived from the TL-23 receptor chain comprises the amino acid sequence set forth in SEQ ID NO: 108. In some embodiments, the nucleotide sequence that encodes the first and/or second intracellular signaling region derived from the TL-23 receptor chain comprises the nucleotide sequence set forth in SEQ ID NO: 109.

In some embodiments, the first and/or second intracellular signaling region derived from the IL-10 receptor alpha chain comprises the amino acid sequence of SEQ ID NO: 118, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 118. In some embodiments, the nucleotide sequence that encodes the first and/or second intracellular signaling region derived from the IL-10 receptor alpha chain comprises the nucleotide sequence that encodes the amino acid sequence of SEQ ID NO: 118, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 118. In some embodiments, the nucleotide sequence that encodes the first and/or second intracellular signaling region derived from the IL-10 receptor alpha chain comprises the nucleotide sequence set forth in SEQ ID NO: 119, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 119. In some embodiments, the first and/or second intracellular signaling region derived from the IL-10 receptor alpha chain comprises the amino acid sequence set forth in SEQ ID NO: 118. In some embodiments, the nucleotide sequence that encodes the first and/or second intracellular signaling region derived from the IL-10 receptor alpha chain comprises the nucleotide sequence set forth in SEQ ID NO: 119.

In some embodiments, the first and/or second intracellular signaling region derived from the glycoprotein 130 (gp130 or IL-6 beta) chain comprises the amino acid sequence of SEQ ID NO: 138, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 138. In certain embodiments, the nucleotide sequence that encodes the first and/or second intracellular signaling region derived from the glycoprotein 130 (gp130 or IL-6 beta) chain comprises the nucleotide sequence that encodes the amino acid sequence of SEQ ID NO: 138, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 138. In certain embodiments, the nucleotide sequence that encodes the first and/or second intracellular signaling region derived from the glycoprotein 130 (gp130 or IL-6 beta) chain comprises the nucleotide sequence set forth in SEQ ID NO: 139, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 139. In certain embodiments, the first and/or second intracellular signaling region derived from the glycoprotein 130 (gp130 or IL-6 beta) chain comprises the amino acid sequence set forth in SEQ ID NO: 138. In certain embodiments, the nucleotide sequence that encodes the first and/or second intracellular signaling region derived from the glycoprotein 130 (gp130 or IL-6 beta) chain comprises the nucleotide sequence set forth in SEQ ID NO: 139.

### First and Second Polypeptides

In some embodiments, the chimeric cytokine receptor may comprise a first polypeptide and a second polypeptide.

In some embodiments, the chimeric cytokine receptor comprises a first polypeptide comprising the amino acid set for in SEQ ID NO: 42, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 42. In certain embodiments, the nucleotide sequence that encodes the first polypeptide of the chimeric cytokine receptor comprises the nucleotide sequence that encodes the amino acid sequence of SEQ ID NO: 42, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 42. In certain embodiments, the nucleotide sequence that encodes the first polypeptide of the chimeric cytokine receptor comprises the nucleotide sequence set forth in SEQ ID NO: 43, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 43. In certain embodiments, the first polypeptide of the chimeric cytokine receptor comprises the amino acid sequence set forth in SEQ ID NO: 42. In certain embodiments, the nucleotide sequence that encodes the first polypeptide of the chimeric cytokine receptor comprises the nucleotide sequence set forth in SEQ ID NO: 43.

In some embodiments, when the chimeric cytokine receptor comprises a first polypeptide comprising the amino acid set for in SEQ ID NO: 42, or a variant thereof, which may be encoded by the nucleotide set forth in SEQ ID NO: 43, or variant thereof, the chimeric cytokine receptor may comprise a second polypeptide comprising the amino acid set for in SEQ ID NO: 40, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 40. In certain embodiments, the nucleotide sequence that encodes the second polypeptide of the chimeric cytokine receptor comprises the nucleotide sequence that encodes the amino acid sequence of SEQ ID NO: 40, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 40. In certain embodiments, the nucleotide sequence that encodes the second polypeptide of the chimeric cytokine receptor comprises the nucleotide sequence set forth in SEQ ID NO: 41, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 41. In certain embodiments, the second polypeptide of the chimeric cytokine receptor comprises the amino acid sequence set forth in SEQ ID NO: 40. In certain embodiments, the nucleotide sequence that encodes the second polypeptide of the chimeric cytokine receptor comprises the nucleotide sequence set forth in SEQ ID NO: 40.

In some embodiments, the first polypeptide of the chimeric cytokine receptor comprises the amino acid sequence of SEQ ID NO: 42, or a sequence having at least 80% identity thereof; and/or the second polypeptide of the chimeric cytokine receptor comprises the amino acid sequence of SEQ ID NO: 40, or a sequence having at least 80% identity thereof.

In some embodiments, when the chimeric cytokine receptor comprises a first polypeptide comprising the amino acid set for in SEQ ID NO: 42, or a variant thereof, which may be encoded by the nucleotide set forth in SEQ ID NO: 43, or variant thereof, the chimeric cytokine receptor may comprise a second polypeptide comprising the amino acid set for in SEQ ID NO: 52, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 52. In certain embodiments, the nucleotide sequence that encodes the second polypeptide of the chimeric cytokine receptor comprises the nucleotide sequence that encodes the amino acid sequence of SEQ ID NO: 52, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 52. In certain embodiments, the nucleotide sequence that encodes the second polypeptide of the chimeric cytokine receptor comprises the nucleotide sequence set forth in SEQ ID NO: 53, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 53. In certain embodiments, the second polypeptide of the chimeric cytokine receptor comprises the amino acid sequence set forth in SEQ ID NO: 52. In certain embodiments, the nucleotide sequence that encodes the second polypeptide of the chimeric cytokine receptor comprises the nucleotide sequence set forth in SEQ ID NO: 53.

In some embodiments, the first polypeptide of the chimeric cytokine receptor comprises the amino acid sequence of SEQ ID NO: 42, or a sequence having at least 80% identity thereof; and/or the second polypeptide of the chimeric cytokine receptor comprises the amino acid sequence of SEQ ID NO: 52, or a sequence having at least 80% identity thereof.

In some embodiments, when the chimeric cytokine receptor comprises a first polypeptide comprising the amino acid set for in SEQ ID NO: 42, or a variant thereof, which may be encoded by the nucleotide set forth in SEQ ID NO: 43, or variant thereof, the chimeric cytokine receptor may comprise a second polypeptide comprising the amino acid set for in SEQ ID NO: 62, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 62. In certain embodiments, the nucleotide sequence that encodes the second polypeptide of the chimeric cytokine receptor comprises the nucleotide sequence that encodes the amino acid sequence of SEQ ID NO: 62, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 62. In certain embodiments, the nucleotide sequence that encodes the second polypeptide of the chimeric cytokine receptor comprises the nucleotide sequence set forth in SEQ ID NO: 63, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 63. In certain embodiments, the second polypeptide of the chimeric cytokine receptor comprises the amino acid sequence set forth in SEQ ID NO: 62. In certain embodiments, the nucleotide sequence that encodes the second polypeptide of the chimeric cytokine receptor comprises the nucleotide sequence set forth in SEQ ID NO: 63.

In some embodiments, the first polypeptide of the chimeric cytokine receptor comprises the amino acid sequence of SEQ ID NO: 42, or a sequence having at least 80% identity thereof; and/or the second polypeptide of the chimeric cytokine receptor comprises the amino acid sequence of SEQ ID NO: 62, or a sequence having at least 80% identity thereof.

In some embodiments, when the chimeric cytokine receptor comprises a first polypeptide comprising the amino acid set for in SEQ ID NO: 42, or a variant thereof, which may be encoded by the nucleotide set forth in SEQ ID NO: 43, or variant thereof, the chimeric cytokine receptor may comprise a second polypeptide comprising the amino acid set for in SEQ ID NO: 72, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 72. In certain embodiments, the nucleotide sequence that encodes the second polypeptide of the chimeric cytokine receptor comprises the nucleotide sequence that encodes the amino acid sequence of SEQ ID NO: 72, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 72. In certain embodiments, the nucleotide sequence that encodes the second polypeptide of the chimeric cytokine receptor comprises the nucleotide sequence set forth in SEQ ID NO: 73, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 73. In certain embodiments, the second polypeptide of the chimeric cytokine receptor comprises the amino acid sequence set forth in SEQ ID NO: 72. In certain embodiments, the nucleotide sequence that encodes the second polypeptide of the chimeric cytokine receptor comprises the nucleotide sequence set forth in SEQ ID NO: 73..

In some embodiments, the first polypeptide of the chimeric cytokine receptor comprises the amino acid sequence of SEQ ID NO: 42, or a sequence having at least 80% identity thereof; and/or the second polypeptide of the chimeric cytokine receptor comprises the amino acid sequence of SEQ ID NO: 72, or a sequence having at least 80% identity thereof.

In some embodiments, when the chimeric cytokine receptor comprises a first polypeptide comprising the amino acid set for in SEQ ID NO: 42, or a variant thereof, which may be encoded by the nucleotide set forth in SEQ ID NO: 43, or variant thereof, the chimeric cytokine receptor may comprise a second polypeptide comprising the amino acid set for in SEQ ID NO: 82, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 82. In certain embodiments, the nucleotide sequence that encodes the second polypeptide of the chimeric cytokine receptor comprises the nucleotide sequence that encodes the amino acid sequence of SEQ ID NO: 82, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 82. In certain embodiments, the nucleotide sequence that encodes the second polypeptide of the chimeric cytokine receptor comprises the nucleotide sequence set forth in SEQ ID NO: 83, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 83. In certain embodiments, the second polypeptide of the chimeric cytokine receptor comprises the amino acid sequence set forth in SEQ ID NO: 82. In certain embodiments, the nucleotide sequence that encodes the second polypeptide of the chimeric cytokine receptor comprises the nucleotide sequence set forth in SEQ ID NO: 83.

In some embodiments, the first polypeptide of the chimeric cytokine receptor comprises the amino acid sequence of SEQ ID NO: 42, or a sequence having at least 80% identity thereof; and/or the second polypeptide of the chimeric cytokine receptor comprises the amino acid sequence of SEQ ID NO: 82, or a sequence having at least 80% identity thereof

In some embodiments, the chimeric cytokine receptor comprises a first polypeptide comprising the amino acid set for in SEQ ID NO: 92, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 92. In certain embodiments, the nucleotide sequence that encodes the first polypeptide of the chimeric cytokine receptor comprises the nucleotide sequence that encodes the amino acid sequence of SEQ ID NO: 92, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 92. In certain embodiments, the nucleotide sequence that encodes the first polypeptide of the chimeric cytokine receptor comprises the nucleotide sequence set forth in SEQ ID NO: 93, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 93. In certain embodiments, the first polypeptide of the chimeric cytokine receptor comprises the amino acid sequence set forth in SEQ ID NO: 92. In certain embodiments, the nucleotide sequence that encodes the first polypeptide of the chimeric cytokine receptor comprises the nucleotide sequence set forth in SEQ ID NO: 93.

In some embodiments, when the chimeric cytokine receptor comprises a first polypeptide comprising the amino acid set for in SEQ ID NO: 92, or a variant thereof, which may be encoded by the nucleotide set forth in SEQ ID NO: 93, or variant thereof, the chimeric cytokine receptor may comprise a second polypeptide comprising the amino acid set for in SEQ ID NO: 102, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 102. In certain embodiments, the nucleotide sequence that encodes the second polypeptide of the chimeric cytokine receptor comprises the nucleotide sequence that encodes the amino acid sequence of SEQ ID NO: 102, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 102. In certain embodiments, the nucleotide sequence that encodes the second polypeptide of the chimeric cytokine receptor comprises the nucleotide sequence set forth in SEQ ID NO: 103, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 103. In certain embodiments, the second polypeptide of the chimeric cytokine receptor comprises the amino acid sequence set forth in SEQ ID NO: 102. In certain embodiments, the nucleotide sequence that encodes the second polypeptide of the chimeric cytokine receptor comprises the nucleotide sequence set forth in SEQ ID NO: 103.

In some embodiments, the first polypeptide of the chimeric cytokine receptor comprises the amino acid sequence of SEQ ID NO: 92, or a sequence having at least 80% identity thereof; and/or the second polypeptide of the chimeric cytokine receptor comprises the amino acid sequence of SEQ ID NO: 102, or a sequence having at least 80% identity thereof.

In some embodiments, when the chimeric cytokine receptor comprises a first polypeptide comprising the amino acid set for in SEQ ID NO: 92, or a variant thereof, which may be encoded by the nucleotide set forth in SEQ ID NO: 93, or variant thereof, the chimeric cytokine receptor may comprise a second polypeptide comprising the amino acid set for in SEQ ID NO: 102, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 102. In certain embodiments, the nucleotide sequence that encodes the second polypeptide of the chimeric cytokine receptor comprises the nucleotide sequence that encodes the amino acid sequence of SEQ ID NO: 102, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 102. In certain embodiments, the nucleotide sequence that encodes the second polypeptide of the chimeric cytokine receptor comprises the nucleotide sequence set forth in SEQ ID NO: 103, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 103. In certain embodiments, the second polypeptide of the chimeric cytokine receptor comprises the amino acid sequence set forth in SEQ ID NO: 102. In certain embodiments, the nucleotide sequence that encodes the second polypeptide of the chimeric cytokine receptor comprises the nucleotide sequence set forth in SEQ ID NO: 103.

In some embodiments, the first polypeptide of the chimeric cytokine receptor comprises the amino acid sequence of SEQ ID NO: 92, or a sequence having at least 80% identity thereof; and/or the second polypeptide of the chimeric cytokine receptor comprises the amino acid sequence of SEQ ID NO: 102, or a sequence having at least 80% identity thereof.

In some embodiments, the chimeric cytokine receptor comprises a first polypeptide comprising the amino acid set for in SEQ ID NO: 112, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 112. In certain embodiments, the nucleotide sequence that encodes the first polypeptide of the chimeric cytokine receptor comprises the nucleotide sequence that encodes the amino acid sequence of SEQ ID NO: 112, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 112. In certain embodiments, the nucleotide sequence that encodes the first polypeptide of the chimeric cytokine receptor comprises the nucleotide sequence set forth in SEQ ID NO: 113, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 113. In certain embodiments, the first polypeptide of the chimeric cytokine receptor comprises the amino acid sequence set forth in SEQ ID NO: 112. In certain embodiments, the nucleotide sequence that encodes the first polypeptide of the chimeric cytokine receptor comprises the nucleotide sequence set forth in SEQ ID NO: 113.

In some embodiments, when the chimeric cytokine receptor comprises a first polypeptide comprising the amino acid set for in SEQ ID NO: 112, or a variant thereof, which may be encoded by the nucleotide set forth in SEQ ID NO: 113, or variant thereof, the chimeric cytokine receptor may comprise a second polypeptide comprising the amino acid set for in SEQ ID NO: 102, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 102. In certain embodiments, the nucleotide sequence that encodes the second polypeptide of the chimeric cytokine receptor comprises the nucleotide sequence that encodes the amino acid sequence of SEQ ID NO: 102, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 102. In certain embodiments, the nucleotide sequence that encodes the second polypeptide of the chimeric cytokine receptor comprises the nucleotide sequence set forth in SEQ ID NO: 103, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 103. In certain embodiments, the second polypeptide of the chimeric cytokine receptor comprises the amino acid sequence set forth in SEQ ID NO: 102. In certain embodiments, the nucleotide sequence that encodes the second polypeptide of the chimeric cytokine receptor comprises the nucleotide sequence set forth in SEQ ID NO: 103.

In some embodiments, the first polypeptide of the chimeric cytokine receptor comprises the amino acid sequence of SEQ ID NO: 112, or a sequence having at least 80% identity thereof; and/or the second polypeptide of the chimeric cytokine receptor comprises the amino acid sequence of SEQ ID NO: 102, or a sequence having at least 80% identity thereof.

In some embodiments, the chimeric cytokine receptor comprises a first polypeptide comprising the amino acid set for in SEQ ID NO: 122 or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 122. In certain embodiments, the nucleotide sequence that encodes the first polypeptide of the chimeric cytokine receptor comprises the nucleotide sequence that encodes the amino acid sequence of SEQ ID NO: 122, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 122. In certain embodiments, the nucleotide sequence that encodes the first polypeptide of the chimeric cytokine receptor comprises the nucleotide sequence set forth in SEQ ID NO: 123, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 123. In certain embodiments, the first polypeptide of the chimeric cytokine receptor comprises the amino acid sequence set forth in SEQ ID NO: 122. In certain embodiments, the nucleotide sequence that encodes the first polypeptide of the chimeric cytokine receptor comprises the nucleotide sequence set forth in SEQ ID NO: 123.

In some embodiments, when the chimeric cytokine receptor comprises a first polypeptide comprising the amino acid set for in SEQ ID NO: 122, or a variant thereof, which may be encoded by the nucleotide set forth in SEQ ID NO: 123, or variant thereof, the chimeric cytokine receptor may comprise a second polypeptide comprising the amino acid set for in SEQ ID NO: 132, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 132. In certain embodiments, the nucleotide sequence that encodes the second polypeptide of the chimeric cytokine receptor comprises the nucleotide sequence that encodes the amino acid sequence of SEQ ID NO: 132, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 132. In certain embodiments, the nucleotide sequence that encodes the second polypeptide of the chimeric cytokine receptor comprises the nucleotide sequence set forth in SEQ ID NO: 133, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 133. In certain embodiments, the second polypeptide of the chimeric cytokine receptor comprises the amino acid sequence set forth in SEQ ID NO: 132. In certain embodiments, the nucleotide sequence that encodes the second polypeptide of the chimeric cytokine receptor comprises the nucleotide sequence set forth in SEQ ID NO: 133.

In some embodiments, the first polypeptide of the chimeric cytokine receptor comprises the amino acid sequence of SEQ ID NO: 122, or a sequence having at least 80% identity thereof; and/or the second polypeptide of the chimeric cytokine receptor comprises the amino acid sequence of SEQ ID NO: 132, or a sequence having at least 80% identity thereof.

In some embodiments, the chimeric cytokine receptor comprises a first polypeptide comprising the amino acid set for in SEQ ID NO: 152, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 152. In certain embodiments, the nucleotide sequence that encodes the first polypeptide of the chimeric cytokine receptor comprises the nucleotide sequence that encodes the amino acid sequence of SEQ ID NO: 152, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 152. In certain embodiments, the nucleotide sequence that encodes the first polypeptide of the chimeric cytokine receptor comprises the nucleotide sequence set forth in SEQ ID NO: 153, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 153. In certain embodiments, the first polypeptide of the chimeric cytokine receptor comprises the amino acid sequence set forth in SEQ ID NO: 152. In certain embodiments, the nucleotide sequence that encodes the first polypeptide of the chimeric cytokine receptor comprises the nucleotide sequence set forth in SEQ ID NO: 153.

In some embodiments, when the chimeric cytokine receptor comprises a first polypeptide comprising the amino acid set for in SEQ ID NO: 152, or a variant thereof, which may be encoded by the nucleotide set forth in SEQ ID NO: 153, or variant thereof, the chimeric cytokine receptor may comprise a second polypeptide comprising the amino acid set for in SEQ ID NO: 142, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 142. In certain embodiments, the nucleotide sequence that encodes the second polypeptide of the chimeric cytokine receptor comprises the nucleotide sequence that encodes the amino acid sequence of SEQ ID NO: 142, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 142. In certain embodiments, the nucleotide sequence that encodes the second polypeptide of the chimeric cytokine receptor comprises the nucleotide sequence set forth in SEQ ID NO: 143, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 143. In certain embodiments, the second polypeptide of the chimeric cytokine receptor comprises the amino acid sequence set forth in SEQ ID NO: 142. In certain embodiments, the nucleotide sequence that encodes the second polypeptide of the chimeric cytokine receptor comprises the nucleotide sequence set forth in SEQ ID NO: 143.

In some embodiments, the first polypeptide of the chimeric cytokine receptor comprises the amino acid sequence of SEQ ID NO: 152, or a sequence having at least 80% identity thereof; and/or the second polypeptide of the chimeric cytokine receptor comprises the amino acid sequence of SEQ ID NO: 142, or a sequence having at least 80% identity thereof.

### Non-Limiting Examples of Chimeric Cytokine Receptor

In some embodiments, the chimeric cytokine receptor comprises
a) a first polypeptide comprising:
   i) a first extracellular region comprising one or more leucine zipper motifs (e.g., two leucine zipper motifs),
   ii) optionally, a first hinge region derived from the common gamma chain (γc),
   iii) a first transmembrane region derived from the common gamma chain (γc), and
   iv) a first intracellular signaling region of the common gamma chain (γc); and
b) a second polypeptide comprising:
   i) a second extracellular region comprising one or more leucine zipper motifs (e.g., two leucine zipper motifs),
   ii) optionally, a second hinge region derived from the IL-2 receptor beta chain,
   iii) a second transmembrane region derived from the IL-2 receptor beta chain, and
   iv) a second intracellular signaling region derived from the IL-2 receptor beta chain.

In some embodiments, the first extracellular region of the chimeric cytokine receptor comprises the amino acid sequence of SEQ ID NO: 158, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 158. In some embodiments, the nucleotide sequence that encodes the first extracellular region of the chimeric cytokine receptor comprises the nucleotide sequence that encodes the amino acid sequence of SEQ ID NO: 158, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 158. In some embodiments, the nucleotide sequence that encodes the first extracellular region of the chimeric cytokine receptor comprises the nucleotide sequence set forth in SEQ ID NO: 159, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 159. In some embodiments, the first extracellular region of the chimeric cytokine receptor comprises the amino acid sequence set forth in SEQ ID NO: 158. In some embodiments, the nucleotide sequence that encodes the first polypeptide of the chimeric cytokine receptor comprises the nucleotide sequence set forth in SEQ ID NO: 159.

In some embodiments, the first hinge region derived from the common gamma chain (γc) comprises the amino acid sequence of SEQ ID NO: 30, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 30. In some embodiments, the nucleotide sequence that encodes the first hinge region derived from the common gamma chain (γc) comprises the nucleotide sequence that encodes the amino acid sequence of SEQ ID NO: 30, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 30. In some embodiments, the nucleotide sequence that encodes the first hinge region derived from the common gamma chain (γc) comprises the nucleotide sequence set forth in SEQ ID NO: 31, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 31. In some embodiments, the first hinge region derived from the common gamma chain (γc) comprises the amino acid sequence set forth in SEQ ID NO: 30. In some embodiments, the nucleotide sequence that encodes the first hinge region derived from the common gamma chain (γc) comprises the nucleotide sequence set forth in SEQ ID NO: 31.

In some embodiments, the first transmembrane region derived from the common gamma chain (γc) comprises the amino acid sequence of SEQ ID NO: 32, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 32. In some embodiments, the nucleotide sequence that encodes the first transmembrane region derived from the common gamma chain (γc) comprises the nucleotide sequence that encodes the amino acid sequence of SEQ ID NO: 32, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 32. In some embodiments, the nucleotide sequence that encodes the first transmembrane region derived from the common gamma chain (γc) comprises the nucleotide sequence set forth in SEQ ID NO: 33, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 33. In some embodiments, the first transmembrane region derived from the common gamma chain (γc) comprises the amino acid sequence set forth in SEQ ID NO: 32. In some embodiments, the nucleotide sequence that encodes the first transmembrane region derived from the common gamma chain (γc) comprises the nucleotide sequence set forth in SEQ ID NO: 33.

In some embodiments, the first intracellular signaling region derived from the common gamma chain (γc) comprises the amino acid sequence of SEQ ID NO: 34, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 34. In some embodiments, the nucleotide sequence that encodes the first intracellular signaling region derived from the common gamma chain (γc) comprises the nucleotide sequence that encodes the amino acid sequence of SEQ ID NO: 34, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 34. In some embodiments, the nucleotide sequence that encodes the first intracellular signaling region derived from the common gamma chain (γc) comprises the nucleotide sequence set forth in SEQ ID NO: 35, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 35. In some embodiments, the first intracellular signaling region derived from the common gamma chain (γc) comprises the amino acid sequence set forth in SEQ ID NO: 34. In some embodiments, the nucleotide sequence that encodes the first intracellular signaling region derived from the common gamma chain (γc) comprises the nucleotide sequence set forth in SEQ ID NO: 35.

In certain embodiment, the second extracellular region of the chimeric cytokine receptor comprises the amino acid sequence of SEQ ID NO: 160, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 160. In certain embodiments, the nucleotide sequence that encodes the second extracellular region of the chimeric cytokine receptor comprises the nucleotide sequence that encodes the amino acid sequence of SEQ ID NO: 160, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 160. In certain embodiments, the nucleotide sequence that encodes the second extracellular region of the chimeric cytokine receptor comprises the nucleotide sequence set forth in SEQ ID NO: 161, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 161. In certain embodiments, the second extracellular region of the chimeric cytokine receptor comprises the amino acid sequence set forth in SEQ ID NO: 160. In certain embodiments, the nucleotide sequence that encodes the second polypeptide of the chimeric cytokine receptor comprises the nucleotide sequence set forth in SEQ ID NO: 161.

In some embodiments, the second hinge region derived from the IL-2 receptor beta chain comprises the amino acid sequence of SEQ ID NO: 14, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 14. In certain embodiments, the nucleotide sequence that encodes the second hinge region derived from the IL-2 receptor beta chain comprises the nucleotide sequence that encodes the amino acid sequence of SEQ ID NO: 14, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 14. In certain embodiments, the nucleotide sequence that encodes the second hinge region derived from the IL-2 receptor beta chain comprises the nucleotide sequence set forth in SEQ ID NO: 15, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 15. In certain embodiments, the second hinge region derived from the IL-2 receptor beta chain comprises the amino acid sequence set forth in SEQ ID NO: 14. In certain embodiments, the nucleotide sequence that encodes the second hinge region derived from the IL-2 receptor beta chain comprises the nucleotide sequence set forth in SEQ ID NO: 15.

In some embodiments, the second transmembrane region derived from the IL-2 receptor beta chain comprises the amino acid sequence of SEQ ID NO: 16, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 16. In certain embodiments, the nucleotide sequence that encodes the second transmembrane region derived from the IL-2 receptor beta chain comprises the nucleotide sequence that encodes the amino acid sequence of SEQ ID NO: 16, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 16. In certain embodiments, the nucleotide sequence that encodes the second transmembrane region derived from the IL-2 receptor beta chain comprises the nucleotide sequence set forth in SEQ ID NO: 17, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 17. In certain embodiments, the second transmembrane region derived from the IL-2 receptor beta chain comprises the amino acid sequence set forth in SEQ ID NO: 16. In certain embodiments, the nucleotide sequence that encodes the second transmembrane region derived from the IL-2 receptor beta chain comprises the nucleotide sequence set forth in SEQ ID NO: 17.

In some embodiments, the second intracellular signaling region derived from the IL-2 receptor beta chain comprises the amino acid sequence of SEQ ID NO: 18, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 18. In certain embodiments, the nucleotide sequence that encodes the second intracellular signaling region derived from the IL-2 receptor beta chain comprises the nucleotide sequence that encodes the amino acid sequence of SEQ ID NO: 18, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 18. In certain embodiments, the nucleotide sequence that encodes the second intracellular signaling region derived from the IL-2 receptor beta chain comprises the nucleotide sequence set forth in SEQ ID NO: 19, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 19. In certain embodiments, the second intracellular signaling region derived from the IL-2 receptor beta chain comprises the amino acid sequence set forth in SEQ ID NO: 18. In certain embodiments, the nucleotide sequence that encodes the second intracellular signaling region derived from the IL-2 receptor beta chain comprises the nucleotide sequence set forth in SEQ ID NO: 19.

In some embodiments, the chimeric cytokine receptor comprises a first polypeptide comprising the amino acid set for in SEQ ID NO: 42, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 42. In certain embodiments, the nucleotide sequence that encodes the first polypeptide of the chimeric cytokine receptor comprises the nucleotide sequence that encodes the amino acid sequence of SEQ ID NO: 42, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 42. In certain embodiments, the nucleotide sequence that encodes the first polypeptide of the chimeric cytokine receptor comprises the nucleotide sequence set forth in SEQ ID NO: 43, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 43. In certain embodiments, the first polypeptide of the chimeric cytokine receptor comprises the amino acid sequence set forth in SEQ ID NO: 42. In certain embodiments, the nucleotide sequence that encodes the first polypeptide of the chimeric cytokine receptor comprises the nucleotide sequence set forth in SEQ ID NO: 43.

In some embodiments, the chimeric cytokine receptor comprises a second polypeptide comprising the amino acid set for in SEQ ID NO: 40, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 40. In certain embodiments, the nucleotide sequence that encodes the second polypeptide of the chimeric cytokine receptor comprises the nucleotide sequence that encodes the amino acid sequence of SEQ ID NO: 40, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 40. In certain embodiments, the nucleotide sequence that encodes the second polypeptide of the chimeric cytokine receptor comprises the nucleotide sequence set forth in SEQ ID NO: 41, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 41. In certain embodiments, the second polypeptide of the chimeric cytokine receptor comprises the amino acid sequence set forth in SEQ ID NO: 40. In certain embodiments, the nucleotide sequence that encodes the second polypeptide of the chimeric cytokine receptor comprises the nucleotide sequence set forth in SEQ ID NO: 40.

In certain embodiments, the first polypeptide may comprise a first extracellular region comprising one or more leucine zipper motifs comprising the amino acid sequence set forth in SEQ ID NO: 158 or variant thereof, which may be encoded by the nucleotide sequence set forth in SEQ ID NO: 159; optionally, a first hinge region of a first cytokine receptor chain comprising the amino acid sequence set forth in SEQ ID NO: 30 or variant thereof, which may be encoded by the nucleotide sequence set forth in SEQ ID NO: 31; a first transmembrane region of a first cytokine receptor chain comprising the amino acid sequence set forth in SEQ ID NO: 32 or variant thereof, which may be encoded by the nucleotide sequence set forth in SEQ ID NO: 33; a first intracellular signaling region of a first cytokine receptor chain comprising the amino acid sequence set forth in SEQ ID NO: 34 or variant thereof, which may be encoded by the nucleotide sequence set forth in SEQ ID NO: 35.

In certain embodiments, the second polypeptide may comprise a second extracellular region comprising one or more leucine zipper motifs comprising the amino acid sequence set forth in SEQ ID NO: 160 or variant thereof, which may be encoded by the nucleotide sequence set forth in SEQ ID NO: 161; optionally, a second hinge region of a second cytokine receptor chain comprising the amino acid sequence set forth in SEQ ID NO: 14 or variant thereof, which may be encoded by the nucleotide sequence set forth in SEQ ID NO: 15; a second transmembrane region of a second cytokine receptor chain comprising the amino acid sequence set forth in SEQ ID NO: 16 or variant thereof, which may be encoded by the nucleotide sequence set forth in SEQ ID NO: 17; a second intracellular signaling region of a second cytokine receptor chain comprising the amino acid sequence set forth in SEQ ID NO: 18 or variant thereof, which may be encoded by the nucleotide sequence set forth in SEQ ID NO: 19.

In some embodiments, when the chimeric cytokine receptor comprises a first polypeptide comprising the amino acid sequence set forth in SEQ ID NO: 42 or a variant thereof, which may be encoded by the nucleotide sequence set forth in SEQ ID NO: 43, and a second polypeptide comprising the amino acid sequence set forth in SEQ ID NO 40, or variant thereof, which may be encoded by the nucleotide sequence set forth in SEQ ID NO: 41, the one or more leucine zipper motifs from first extracellular region may heterodimerize with the one or more leucine zipper motifs from the second extracellular region. In certain embodiments, the chimeric cytokine receptor comprises a first polypeptide comprising the amino acid sequence set forth in SEQ ID NO: 42 or a variant thereof, and a second polypeptide comprising the amino acid sequence set forth in SEQ ID NO 40 or variant thereof. In certain embodiments, the nucleotide sequence that encodes the first polypeptide of the chimeric cytokine receptor comprises the nucleotide sequence set forth in SEQ ID NO: 43. In certain embodiments, the nucleotide sequence that encodes the second polypeptide of the chimeric cytokine receptor comprises the nucleotide sequence set forth in SEQ ID NO: 41.

In some embodiments, the chimeric cytokine receptor comprises
a) a first polypeptide comprising:
   i) a first extracellular region comprising one or more leucine zipper motifs (e.g., two leucine zipper motifs),
   ii) optionally, a first hinge region derived from the common gamma chain (γc),
   iii) a first transmembrane region derived from the common gamma chain (γc), and
   iv) a first intracellular signaling region of the common gamma chain (γc); and
b) a second polypeptide comprising:
   i) a second extracellular region comprising one or more leucine zipper motifs (e.g., two leucine zipper motifs),
   ii) optionally, a second hinge region derived from the IL-7 receptor alpha chain,
   iii) a second transmembrane region derived from the IL-7 receptor alpha chain, and
   iv) a second intracellular signaling region derived from the IL-7 receptor alpha chain.

In some embodiments, the first extracellular region of the chimeric cytokine receptor comprises the amino acid sequence of SEQ ID NO: 158, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 158. In some embodiments, the nucleotide sequence that encodes the first extracellular region of the chimeric cytokine receptor comprises the nucleotide sequence that encodes the amino acid sequence of SEQ ID NO: 158, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 158. In some embodiments, the nucleotide sequence that encodes the first extracellular region of the chimeric cytokine receptor comprises the nucleotide sequence set forth in SEQ ID NO: 159, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 159. In some embodiments, the first extracellular region of the chimeric cytokine receptor comprises the amino acid sequence set forth in SEQ ID NO: 158. In some embodiments, the nucleotide sequence that encodes the first polypeptide of the chimeric cytokine receptor comprises the nucleotide sequence set forth in SEQ ID NO: 159.

In some embodiments, the first hinge region derived from the common gamma chain (7c) comprises the amino acid sequence of SEQ ID NO: 30, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 30. In some embodiments, the nucleotide sequence that encodes the first hinge region derived from the common gamma chain (γc) comprises the nucleotide sequence that encodes the amino acid sequence of SEQ ID NO: 30, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 30. In some embodiments, the nucleotide sequence that encodes the first hinge region derived from the common gamma chain (γc) comprises the nucleotide sequence set forth in SEQ ID NO: 31, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 31. In some embodiments, the first hinge region derived from the common gamma chain (γc) comprises the amino acid sequence set forth in SEQ ID NO: 30. In some embodiments, the nucleotide sequence that encodes the first hinge region derived from the common gamma chain (γc) comprises the nucleotide sequence set forth in SEQ ID NO: 31.

In some embodiments, the first transmembrane region derived from the common gamma chain (γc) comprises the amino acid sequence of SEQ ID NO: 32, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 32. In some embodiments, the nucleotide sequence that encodes the first transmembrane region derived from the common gamma chain (γc) comprises the nucleotide sequence that encodes the amino acid sequence of SEQ ID NO: 32, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 32. In some embodiments, the nucleotide sequence that encodes the first transmembrane region derived from the common gamma chain (γc) comprises the nucleotide sequence set forth in SEQ ID NO: 33, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 33. In some embodiments, the first transmembrane region derived from the common gamma chain (γc) comprises the amino acid sequence set forth in SEQ ID NO: 32. In some embodiments, the nucleotide sequence that encodes the first transmembrane region derived from the common gamma chain (γc) comprises the nucleotide sequence set forth in SEQ ID NO: 33.

In some embodiments, the first intracellular signaling region derived from the common gamma chain (γc) comprises the amino acid sequence of SEQ ID NO: 34, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 34. In some embodiments, the nucleotide sequence that encodes the first intracellular signaling region derived from the common gamma chain (γc) comprises the nucleotide sequence that encodes the amino acid sequence of SEQ ID NO: 34, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 34. In some embodiments, the nucleotide sequence that encodes the first intracellular signaling region derived from the common gamma chain (γc) comprises the nucleotide sequence set forth in SEQ ID NO: 35, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 35. In some embodiments, the first intracellular signaling region derived from the common gamma chain (γc) comprises the amino acid sequence set forth in SEQ ID NO: 34. In some embodiments, the nucleotide sequence that encodes the first intracellular signaling region derived from the common gamma chain (γc) comprises the nucleotide sequence set forth in SEQ ID NO: 35

In certain embodiment, the second extracellular region of the chimeric cytokine receptor comprises the amino acid sequence of SEQ ID NO: 160, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 160. In certain embodiments, the nucleotide sequence that encodes the second extracellular region of the chimeric cytokine receptor comprises the nucleotide sequence that encodes the amino acid sequence of SEQ ID NO: 160, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 160. In certain embodiments, the nucleotide sequence that encodes the second extracellular region of the chimeric cytokine receptor comprises the nucleotide sequence set forth in SEQ ID NO: 161, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 161. In certain embodiments, the second extracellular region of the chimeric cytokine receptor comprises the amino acid sequence set forth in SEQ ID NO: 160. In certain embodiments, the nucleotide sequence that encodes the second polypeptide of the chimeric cytokine receptor comprises the nucleotide sequence set forth in SEQ ID NO: 161.

In some embodiments, the second hinge region derived from the IL-7 receptor alpha chain comprises the amino acid sequence of SEQ ID NO: 44, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 44. In certain embodiments, the nucleotide sequence that encodes the second hinge region derived from the IL-7 receptor alpha chain comprises the nucleotide sequence that encodes the amino acid sequence of SEQ ID NO: 44, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 44. In certain embodiments, the nucleotide sequence that encodes the second hinge region derived from the IL-7 receptor alpha chain comprises the nucleotide sequence set forth in SEQ ID NO: 45, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 45. In certain embodiments, the second hinge region derived from the IL-7 receptor alpha chain comprises the amino acid sequence set forth in SEQ ID NO: 44. In certain embodiments, the nucleotide sequence that encodes the second hinge region derived from the IL-7 receptor alpha chain comprises the nucleotide sequence set forth in SEQ ID NO: 45.

In some embodiments, the second transmembrane region derived from the IL-7 receptor alpha chain comprises the amino acid sequence of SEQ ID NO: 46, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 46. In certain embodiments, the nucleotide sequence that encodes the second transmembrane region derived from the IL-7 receptor alpha chain comprises the nucleotide sequence that encodes the amino acid sequence of SEQ ID NO: 46, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 46. In certain embodiments, the nucleotide sequence that encodes the second transmembrane region derived from the IL-7 receptor alpha chain comprises the nucleotide sequence set forth in SEQ ID NO: 47, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 47. In certain embodiments, the second transmembrane region derived from the TL-7 receptor alpha chain comprises the amino acid sequence set forth in SEQ ID NO: 46. In certain embodiments, the nucleotide sequence that encodes the second transmembrane region derived from the IL-7 receptor alpha chain comprises the nucleotide sequence set forth in SEQ ID NO: 47.

In some embodiments, the second intracellular signaling region derived from the IL-7 receptor alpha chain comprises the amino acid sequence of SEQ ID NO: 48, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 48. In certain embodiments, the nucleotide sequence that encodes the second intracellular signaling region derived from the IL-7 receptor alpha chain comprises the nucleotide sequence that encodes the amino acid sequence of SEQ ID NO: 48, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 48. In certain embodiments, the nucleotide sequence that encodes the second intracellular signaling region derived from the IL-7 receptor alpha chain comprises the nucleotide sequence set forth in SEQ ID NO: 49, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 49. In certain embodiments, the second intracellular signaling region derived from the IL-7 receptor alpha chain comprises the amino acid sequence set forth in SEQ ID NO: 48. In certain embodiments, the nucleotide sequence that encodes the second intracellular signaling region derived from the IL-7 receptor alpha chain comprises the nucleotide sequence set forth in SEQ ID NO: 49.

In some embodiments, the chimeric cytokine receptor comprises a first polypeptide comprising the amino acid set for in SEQ ID NO: 42, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 42. In certain embodiments, the nucleotide sequence that encodes the first polypeptide of the chimeric cytokine receptor comprises the nucleotide sequence that encodes the amino acid sequence of SEQ ID NO: 42, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 42. In certain embodiments, the nucleotide sequence that encodes the first polypeptide of the chimeric cytokine receptor comprises the nucleotide sequence set forth in SEQ ID NO: 43, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 43. In certain embodiments, the first polypeptide of the chimeric cytokine receptor comprises the amino acid sequence set forth in SEQ ID NO: 42. In certain embodiments, the nucleotide sequence that encodes the first polypeptide of the chimeric cytokine receptor comprises the nucleotide sequence set forth in SEQ ID NO: 43.

In some embodiments, the chimeric cytokine receptor comprises a second polypeptide comprising the amino acid set for in SEQ ID NO: 52, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 52. In certain embodiments, the nucleotide sequence that encodes the second polypeptide of the chimeric cytokine receptor comprises the nucleotide sequence that encodes the amino acid sequence of SEQ ID NO: 52, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 52. In certain embodiments, the nucleotide sequence that encodes the second polypeptide of the chimeric cytokine receptor comprises the nucleotide sequence set forth in SEQ ID NO: 53, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 53. In certain embodiments, the second polypeptide of the chimeric cytokine receptor comprises the amino acid sequence set forth in SEQ ID NO: 52. In certain embodiments, the nucleotide sequence that encodes the second polypeptide of the chimeric cytokine receptor comprises the nucleotide sequence set forth in SEQ ID NO: 53.

In certain embodiments, the first polypeptide may comprise a first extracellular region comprising one or more leucine zipper motifs comprising the amino acid sequence set forth in SEQ ID NO: 158 or variant thereof, which may be encoded by the nucleotide sequence set forth in SEQ ID NO: 159; optionally, a first hinge region of a first cytokine receptor chain comprising the amino acid sequence set forth in SEQ ID NO: 30 or variant thereof, which may be encoded by the nucleotide sequence set forth in SEQ ID NO: 31; a first transmembrane region of a first cytokine receptor chain comprising the amino acid sequence set forth in SEQ ID NO: 32 or variant thereof, which may be encoded by the nucleotide sequence set forth in SEQ ID NO: 33; a first intracellular signaling region of a first cytokine receptor chain comprising the amino acid sequence set forth in SEQ ID NO: 34 or variant thereof, which may be encoded by the nucleotide sequence set forth in SEQ ID NO: 35.

In certain embodiments, the second polypeptide may comprise a second extracellular region comprising one or more leucine zipper motifs comprising the amino acid sequence set forth in SEQ ID NO: 160 or variant thereof, which may be encoded by the nucleotide sequence set forth in SEQ ID NO: 161; optionally, a second hinge region of a second cytokine receptor chain comprising the amino acid sequence set forth in SEQ ID NO: 44 or variant thereof, which may be encoded by the nucleotide sequence set forth in SEQ ID NO: 45; a second transmembrane region of a second cytokine receptor chain comprising the amino acid sequence set forth in SEQ ID NO: 46 or variant thereof, which may be encoded by the nucleotide sequence set forth in SEQ ID NO: 47; a second intracellular signaling region of a second cytokine receptor chain comprising the amino acid sequence set forth in SEQ ID NO: 48 or variant thereof, which may be encoded by the nucleotide sequence set forth in SEQ ID NO: 49.

In some embodiments, when the chimeric cytokine receptor comprises a first polypeptide comprising the amino acid sequence set forth in SEQ ID NO: 42 or a variant thereof, which may be encoded by the nucleotide sequence set forth in SEQ ID NO: 43, and a second polypeptide comprising the amino acid sequence set forth in SEQ ID NO 52, or variant thereof, which may be encoded by the nucleotide sequence set forth in SEQ ID NO: 53, the one or more leucine zipper motifs from first extracellular region may heterodimerize with the one or more leucine zipper motifs from the second extracellular region. In certain embodiments, the chimeric cytokine receptor comprises a first polypeptide comprising the amino acid sequence set forth in SEQ ID NO: 42 or a variant thereof, and a second polypeptide comprising the amino acid sequence set forth in SEQ ID NO 52, or variant thereof. In certain embodiments, the nucleotide sequence that encodes the first polypeptide of the chimeric cytokine receptor comprises the nucleotide sequence set forth in SEQ ID NO: 43. In certain embodiments, the nucleotide sequence that encodes the second polypeptide of the chimeric cytokine receptor comprises the nucleotide sequence set forth in SEQ ID NO: 53.

In some embodiments, the chimeric cytokine receptor comprises
a) a first polypeptide comprising:
   i) a first extracellular region comprising one or more leucine zipper motifs (e.g., two leucine zipper motifs),
   ii) optionally, a first hinge region derived from the common gamma chain (γc),
   iii) a first transmembrane region derived from the common gamma chain (γc), and
   iv) a first intracellular signaling region of the common gamma chain (γc); and
b) a second polypeptide comprising:
   i) a second extracellular region comprising one or more leucine zipper motifs (e.g., two leucine zipper motifs),
   ii) optionally, a second hinge region derived from the IL-21 receptor chain,
   iii) a second transmembrane region derived from the IL-21 receptor chain, and
   iv) a second intracellular signaling region derived from the IL-21 receptor chain.

In some embodiments, the first extracellular region of the chimeric cytokine receptor comprises the amino acid sequence of SEQ ID NO: 158, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 158. In some embodiments, the nucleotide sequence that encodes the first extracellular region of the chimeric cytokine receptor comprises the nucleotide sequence that encodes the amino acid sequence of SEQ ID NO: 158, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 158. In some embodiments, the nucleotide sequence that encodes the first extracellular region of the chimeric cytokine receptor comprises the nucleotide sequence set forth in SEQ ID NO: 159, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 159. In some embodiments, the first extracellular region of the chimeric cytokine receptor comprises the amino acid sequence set forth in SEQ ID NO: 158. In some embodiments, the nucleotide sequence that encodes the first polypeptide of the chimeric cytokine receptor comprises the nucleotide sequence set forth in SEQ ID NO: 159.

In some embodiments, the first hinge region derived from the common gamma chain (γc) comprises the amino acid sequence of SEQ ID NO: 30, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 30. In some embodiments, the nucleotide sequence that encodes the first hinge region derived from the common gamma chain (γc) comprises the nucleotide sequence that encodes the amino acid sequence of SEQ ID NO: 30, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 30. In some embodiments, the nucleotide sequence that encodes the first hinge region derived from the common gamma chain (γc) comprises the nucleotide sequence set forth in SEQ ID NO: 31, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 31. In some embodiments, the first hinge region derived from the common gamma chain (γc) comprises the amino acid sequence set forth in SEQ ID NO: 30. In some embodiments, the nucleotide sequence that encodes the first hinge region derived from the common gamma chain (γc) comprises the nucleotide sequence set forth in SEQ ID NO: 31.

In some embodiments, the first transmembrane region derived from the common gamma chain (γc) comprises the amino acid sequence of SEQ ID NO: 32, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 32. In some embodiments, the nucleotide sequence that encodes the first transmembrane region derived from the common gamma chain (γc) comprises the nucleotide sequence that encodes the amino acid sequence of SEQ ID NO: 32, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 32. In some embodiments, the nucleotide sequence that encodes the first transmembrane region derived from the common gamma chain (γc) comprises the nucleotide sequence set forth in SEQ ID NO: 33, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 33. In some embodiments, the first transmembrane region derived from the common gamma chain (γc) comprises the amino acid sequence set forth in SEQ ID NO: 32. In some embodiments, the nucleotide sequence that encodes the first transmembrane region derived from the common gamma chain (γc) comprises the nucleotide sequence set forth in SEQ ID NO: 33.

In some embodiments, the first intracellular signaling region derived from the common gamma chain (γc) comprises the amino acid sequence of SEQ ID NO: 34, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 34. In some embodiments, the nucleotide sequence that encodes the first intracellular signaling region derived from the common gamma chain (γc) comprises the nucleotide sequence that encodes the amino acid sequence of SEQ ID NO: 34, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 34. In some embodiments, the nucleotide sequence that encodes the first intracellular signaling region derived from the common gamma chain (γc) comprises the nucleotide sequence set forth in SEQ ID NO: 35, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 35. In some embodiments, the first intracellular signaling region derived from the common gamma chain (γc) comprises the amino acid sequence set forth in SEQ ID NO: 34. In some embodiments, the nucleotide sequence that encodes the first intracellular signaling region derived from the common gamma chain (γc) comprises the nucleotide sequence set forth in SEQ ID NO: 35

In certain embodiment, the second extracellular region of the chimeric cytokine receptor comprises the amino acid sequence of SEQ ID NO: 160, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 160. In certain embodiments, the nucleotide sequence that encodes the second extracellular region of the chimeric cytokine receptor comprises the nucleotide sequence that encodes the amino acid sequence of SEQ ID NO: 160, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 160. In certain embodiments, the nucleotide sequence that encodes the second extracellular region of the chimeric cytokine receptor comprises the nucleotide sequence set forth in SEQ ID NO: 161, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 161. In certain embodiments, the second extracellular region of the chimeric cytokine receptor comprises the amino acid sequence set forth in SEQ ID NO: 160. In certain embodiments, the nucleotide sequence that encodes the second polypeptide of the chimeric cytokine receptor comprises the nucleotide sequence set forth in SEQ ID NO: 161.

In some embodiments, the second hinge region derived from the IL-21 receptor chain comprises the amino acid sequence of SEQ ID NO: 54, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 54. In certain embodiments, the nucleotide sequence that encodes the second hinge region derived from the IL-21 receptor chain comprises the nucleotide sequence that encodes the amino acid sequence of SEQ ID NO: 54, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 54. In certain embodiments, the nucleotide sequence that encodes the second hinge region derived from the IL-21 receptor chain comprises the nucleotide sequence set forth in SEQ ID NO: 55, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 55. In certain embodiments, the second hinge region derived from the IL-21 receptor chain comprises the amino acid sequence set forth in SEQ ID NO: 54. In certain embodiments, the nucleotide sequence that encodes the second hinge region derived from the IL-21 receptor chain comprises the nucleotide sequence set forth in SEQ ID NO: 55.

In some embodiments, the second transmembrane region derived from the IL-21 receptor chain comprises the amino acid sequence of SEQ ID NO: 56, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 56. In certain embodiments, the nucleotide sequence that encodes the second transmembrane region derived from the IL-21 receptor chain comprises the nucleotide sequence that encodes the amino acid sequence of SEQ ID NO: 56, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 56. In certain embodiments, the nucleotide sequence that encodes the second transmembrane region derived from the IL-21 receptor chain comprises the nucleotide sequence set forth in SEQ ID NO: 57, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 57. In certain embodiments, the second transmembrane region derived from the IL-21 receptor chain comprises the amino acid sequence set forth in SEQ ID NO: 56. In certain embodiments, the nucleotide sequence that encodes the second transmembrane region derived from the IL-21 receptor chain comprises the nucleotide sequence set forth in SEQ ID NO: 57.

In some embodiments, the second intracellular signaling region derived from the IL-21 receptor chain comprises the amino acid sequence of SEQ ID NO: 58, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 58. In certain embodiments, the nucleotide sequence that encodes the second intracellular signaling region derived from the IL-21 receptor chain comprises the nucleotide sequence that encodes the amino acid sequence of SEQ ID NO: 58, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 58. In certain embodiments, the nucleotide sequence that encodes the second intracellular signaling region derived from the IL-21 receptor chain comprises the nucleotide sequence set forth in SEQ ID NO: 59, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 59. In certain embodiments, the second intracellular signaling region derived from the IL-21 receptor chain comprises the amino acid sequence set forth in SEQ ID NO: 58. In certain embodiments, the nucleotide sequence that encodes the second intracellular signaling region derived from the IL-21 receptor chain comprises the nucleotide sequence set forth in SEQ ID NO: 59.

In some embodiments, the chimeric cytokine receptor comprises a first polypeptide comprising the amino acid set for in SEQ ID NO: 42, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 42. In certain embodiments, the nucleotide sequence that encodes the first polypeptide of the chimeric cytokine receptor comprises the nucleotide sequence that encodes the amino acid sequence of SEQ ID NO: 42, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 42. In certain embodiments, the nucleotide sequence that encodes the first polypeptide of the chimeric cytokine receptor comprises the nucleotide sequence set forth in SEQ ID NO: 43, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 43. In certain embodiments, the first polypeptide of the chimeric cytokine receptor comprises the amino acid sequence set forth in SEQ ID NO: 42. In certain embodiments, the nucleotide sequence that encodes the first polypeptide of the chimeric cytokine receptor comprises the nucleotide sequence set forth in SEQ ID NO: 43.

In some embodiments, the chimeric cytokine receptor comprises a second polypeptide comprising the amino acid set for in SEQ ID NO: 62, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 62. In certain embodiments, the nucleotide sequence that encodes the second polypeptide of the chimeric cytokine receptor comprises the nucleotide sequence that encodes the amino acid sequence of SEQ ID NO: 62, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 62. In certain embodiments, the nucleotide sequence that encodes the second polypeptide of the chimeric cytokine receptor comprises the nucleotide sequence set forth in SEQ ID NO: 63, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 63. In certain embodiments, the second polypeptide of the chimeric cytokine receptor comprises the amino acid sequence set forth in SEQ ID NO: 62. In certain embodiments, the nucleotide sequence that encodes the second polypeptide of the chimeric cytokine receptor comprises the nucleotide sequence set forth in SEQ ID NO: 63.

In certain embodiments, the first polypeptide may comprise a first extracellular region comprising one or more leucine zipper motifs comprising the amino acid sequence set forth in SEQ ID NO: 158 or variant thereof, which may be encoded by the nucleotide sequence set forth in SEQ ID NO: 159; optionally, a first hinge region of a first cytokine receptor chain comprising the amino acid sequence set forth in SEQ ID NO: 30 or variant thereof, which may be encoded by the nucleotide sequence set forth in SEQ ID NO: 31; a first transmembrane region of a first cytokine receptor chain comprising the amino acid sequence set forth in SEQ ID NO: 32 or variant thereof, which may be encoded by the nucleotide sequence set forth in SEQ ID NO: 33; a first intracellular signaling region of a first cytokine receptor chain comprising the amino acid sequence set forth in SEQ ID NO: 34 or variant thereof, which may be encoded by the nucleotide sequence set forth in SEQ ID NO: 35.

In certain embodiments, the second polypeptide may comprise a second extracellular region comprising one or more leucine zipper motifs comprising the amino acid sequence set forth in SEQ ID NO: 160 or variant thereof, which may be encoded by the nucleotide sequence set forth in SEQ ID NO: 161; optionally, a second hinge region of a second cytokine receptor chain comprising the amino acid sequence set forth in SEQ ID NO: 54 or variant thereof, which may be encoded by the nucleotide sequence set forth in SEQ ID NO: 55; a second transmembrane region of a second cytokine receptor chain comprising the amino acid sequence set forth in SEQ ID NO: 56 or variant thereof, which may be encoded by the nucleotide sequence set forth in SEQ ID NO: 57; a second intracellular signaling region of a second cytokine receptor chain comprising the amino acid sequence set forth in SEQ ID NO: 58 or variant thereof, which may be encoded by the nucleotide sequence set forth in SEQ ID NO: 59.

In some embodiments, when the chimeric cytokine receptor comprises a first polypeptide comprising the amino acid sequence set forth in SEQ ID NO: 42 or a variant thereof, which may be encoded by the nucleotide sequence set forth in SEQ ID NO: 43, and a second polypeptide comprising the amino acid sequence set forth in SEQ ID NO 62, or variant thereof, which may be encoded by the nucleotide sequence set forth in SEQ ID NO: 63, the one or more leucine zipper motifs from first extracellular region may heterodimerize with the one or more leucine zipper motifs from the second extracellular region. In certain embodiments, the chimeric cytokine receptor comprises a first polypeptide comprising the amino acid sequence set forth in SEQ ID NO: 42 or a variant thereof, and a second polypeptide comprising the amino acid sequence set forth in SEQ ID NO 62, or variant thereof. In certain embodiments, the nucleotide sequence that encodes the first polypeptide of the chimeric cytokine receptor comprises the nucleotide sequence set forth in SEQ ID NO: 43. In certain embodiments, the nucleotide sequence that encodes the second polypeptide of the chimeric cytokine receptor comprises the nucleotide sequence set forth in SEQ ID NO: 63.

In some embodiments, the chimeric cytokine receptor comprises
a) a first polypeptide comprising:
   i) a first extracellular region comprising one or more leucine zipper motifs (e.g., two leucine zipper motifs),
   ii) optionally, a first hinge region derived from the common gamma chain (γc),
   iii) a first transmembrane region derived from the common gamma chain (γc), and
   iv) a first intracellular signaling region of the common gamma chain (γc); and
b) a second polypeptide comprising:
   i) a second extracellular region comprising one or more leucine zipper motifs (e.g., two leucine zipper motifs),
   ii) optionally, a second hinge region derived from the IL-9 receptor chain,
   iii) a second transmembrane region derived from the IL-9 receptor chain, and
   iv) a second intracellular signaling region derived from the IL-9 receptor chain.

In some embodiments, the first extracellular region of the chimeric cytokine receptor comprises the amino acid sequence of SEQ ID NO: 158, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 158. In some embodiments, the nucleotide sequence that encodes the first extracellular region of the chimeric cytokine receptor comprises the nucleotide sequence that encodes the amino acid sequence of SEQ ID NO: 158, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 158. In some embodiments, the nucleotide sequence that encodes the first extracellular region of the chimeric cytokine receptor comprises the nucleotide sequence set forth in SEQ ID NO: 159, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 159. In some embodiments, the first extracellular region of the chimeric cytokine receptor comprises the amino acid sequence set forth in SEQ ID NO: 158. In some embodiments, the nucleotide sequence that encodes the first polypeptide of the chimeric cytokine receptor comprises the nucleotide sequence set forth in SEQ ID NO: 159.

In some embodiments, the first hinge region derived from the common gamma chain (γc) comprises the amino acid sequence of SEQ ID NO: 30, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 30. In some embodiments, the nucleotide sequence that encodes the first hinge region derived from the common gamma chain (γc) comprises the nucleotide sequence that encodes the amino acid sequence of SEQ ID NO: 30, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 30. In some embodiments, the nucleotide sequence that encodes the first hinge region derived from the common gamma chain (γc) comprises the nucleotide sequence set forth in SEQ ID NO: 31, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 31. In some embodiments, the first hinge region derived from the common gamma chain (γc) comprises the amino acid sequence set forth in SEQ ID NO: 30. In some embodiments, the nucleotide sequence that encodes the first hinge region derived from the common gamma chain (γc) comprises the nucleotide sequence set forth in SEQ ID NO: 31.

In some embodiments, the first transmembrane region derived from the common gamma chain (γc) comprises the amino acid sequence of SEQ ID NO: 32, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 32. In some embodiments, the nucleotide sequence that encodes the first transmembrane region derived from the common gamma chain (γc) comprises the nucleotide sequence that encodes the amino acid sequence of SEQ ID NO: 32, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 32. In some embodiments, the nucleotide sequence that encodes the first transmembrane region derived from the common gamma chain (γc) comprises the nucleotide sequence set forth in SEQ ID NO: 33, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 33. In some embodiments, the first transmembrane region derived from the common gamma chain (γc) comprises the amino acid sequence set forth in SEQ ID NO: 32. In some embodiments, the nucleotide sequence that encodes the first transmembrane region derived from the common gamma chain (γc) comprises the nucleotide sequence set forth in SEQ ID NO: 33.

In some embodiments, the first intracellular signaling region derived from the common gamma chain (γc) comprises the amino acid sequence of SEQ ID NO: 34, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 34. In some embodiments, the nucleotide sequence that encodes the first intracellular signaling region derived from the common gamma chain (γc) comprises the nucleotide sequence that encodes the amino acid sequence of SEQ ID NO: 34, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 34. In some embodiments, the nucleotide sequence that encodes the first intracellular signaling region derived from the common gamma chain (γc) comprises the nucleotide sequence set forth in SEQ ID NO: 35, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 35. In some embodiments, the first intracellular signaling region derived from the common gamma chain (γc) comprises the amino acid sequence set forth in SEQ ID NO: 34. In some embodiments, the nucleotide sequence that encodes the first intracellular signaling region derived from the common gamma chain (γc) comprises the nucleotide sequence set forth in SEQ ID NO: 35

In certain embodiment, the second extracellular region of the chimeric cytokine receptor comprises the amino acid sequence of SEQ ID NO: 160, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 160. In certain embodiments, the nucleotide sequence that encodes the second extracellular region of the chimeric cytokine receptor comprises the nucleotide sequence that encodes the amino acid sequence of SEQ ID NO: 160, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 160. In certain embodiments, the nucleotide sequence that encodes the second extracellular region of the chimeric cytokine receptor comprises the nucleotide sequence set forth in SEQ ID NO: 161, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 161. In certain embodiments, the second extracellular region of the chimeric cytokine receptor comprises the amino acid sequence set forth in SEQ ID NO: 160. In certain embodiments, the nucleotide sequence that encodes the second polypeptide of the chimeric cytokine receptor comprises the nucleotide sequence set forth in SEQ ID NO: 161.

In some embodiments, the second hinge region derived from the IL-9 receptor chain comprises the amino acid sequence of SEQ ID NO: 64, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 64. In certain embodiments, the nucleotide sequence that encodes the second hinge region derived from the IL-9 receptor chain comprises the nucleotide sequence that encodes the amino acid sequence of SEQ ID NO: 64, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 64. In certain embodiments, the nucleotide sequence that encodes the second hinge region derived from the IL-9 receptor chain comprises the nucleotide sequence set forth in SEQ ID NO: 65, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 65. In certain embodiments, the second hinge region derived from the IL-9 receptor chain comprises the amino acid sequence set forth in SEQ ID NO: 64. In certain embodiments, the nucleotide sequence that encodes the second hinge region derived from the IL-9 receptor chain comprises the nucleotide sequence set forth in SEQ ID NO: 65.

In some embodiments, the second transmembrane region derived from the IL-9 receptor chain comprises the amino acid sequence of SEQ ID NO: 66, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 66. In certain embodiments, the nucleotide sequence that encodes the second transmembrane region derived from the IL-9 receptor chain comprises the nucleotide sequence that encodes the amino acid sequence of SEQ ID NO: 66, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 66. In certain embodiments, the nucleotide sequence that encodes the second transmembrane region derived from the IL-9 receptor chain comprises the nucleotide sequence set forth in SEQ ID NO: 67, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 67. In certain embodiments, the second transmembrane region derived from the IL-9 receptor chain comprises the amino acid sequence set forth in SEQ ID NO: 66. In certain embodiments, the nucleotide sequence that encodes the second transmembrane region derived from the IL-9 receptor chain comprises the nucleotide sequence set forth in SEQ ID NO: 67.

In some embodiments, the second intracellular signaling region derived from the IL-9 receptor chain comprises the amino acid sequence of SEQ ID NO: 68, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 68. In certain embodiments, the nucleotide sequence that encodes the second intracellular signaling region derived from the IL-9 receptor chain comprises the nucleotide sequence that encodes the amino acid sequence of SEQ ID NO: 68, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 68. In certain embodiments, the nucleotide sequence that encodes the second intracellular signaling region derived from the IL-9 receptor chain comprises the nucleotide sequence set forth in SEQ ID NO: 69, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 69. In certain embodiments, the second intracellular signaling region derived from the IL-9 receptor chain comprises the amino acid sequence set forth in SEQ ID NO: 68. In certain embodiments, the nucleotide sequence that encodes the second intracellular signaling region derived from the IL-9 receptor chain comprises the nucleotide sequence set forth in SEQ ID NO: 69.

In some embodiments, the chimeric cytokine receptor comprises a first polypeptide comprising the amino acid set for in SEQ ID NO: 42, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 42. In certain embodiments, the nucleotide sequence that encodes the first polypeptide of the chimeric cytokine receptor comprises the nucleotide sequence that encodes the amino acid sequence of SEQ ID NO: 42, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 42. In certain embodiments, the nucleotide sequence that encodes the first polypeptide of the chimeric cytokine receptor comprises the nucleotide sequence set forth in SEQ ID NO: 43, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 43. In certain embodiments, the first polypeptide of the chimeric cytokine receptor comprises the amino acid sequence set forth in SEQ ID NO: 42. In certain embodiments, the nucleotide sequence that encodes the first polypeptide of the chimeric cytokine receptor comprises the nucleotide sequence set forth in SEQ ID NO: 43.

In some embodiments, the chimeric cytokine receptor comprises a second polypeptide comprising the amino acid set for in SEQ ID NO: 72, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 72. In certain embodiments, the nucleotide sequence that encodes the second polypeptide of the chimeric cytokine receptor comprises the nucleotide sequence that encodes the amino acid sequence of SEQ ID NO: 72, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 72. In certain embodiments, the nucleotide sequence that encodes the second polypeptide of the chimeric cytokine receptor comprises the nucleotide sequence set forth in SEQ ID NO: 73, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 73. In certain embodiments, the second polypeptide of the chimeric cytokine receptor comprises the amino acid sequence set forth in SEQ ID NO: 72. In certain embodiments, the nucleotide sequence that encodes the second polypeptide of the chimeric cytokine receptor comprises the nucleotide sequence set forth in SEQ ID NO: 73.

In certain embodiments, the first polypeptide may comprise a first extracellular region comprising one or more leucine zipper motifs comprising the amino acid sequence set forth in SEQ ID NO: 158 or variant thereof, which may be encoded by the nucleotide sequence set forth in SEQ ID NO: 159; optionally, a first hinge region of a first cytokine receptor chain comprising the amino acid sequence set forth in SEQ ID NO: 30 or variant thereof, which may be encoded by the nucleotide sequence set forth in SEQ ID NO: 31; a first transmembrane region of a first cytokine receptor chain comprising the amino acid sequence set forth in SEQ ID NO: 32 or variant thereof, which may be encoded by the nucleotide sequence set forth in SEQ ID NO: 33; a first intracellular signaling region of a first cytokine receptor chain comprising the amino acid sequence set forth in SEQ ID NO: 34 or variant thereof, which may be encoded by the nucleotide sequence set forth in SEQ ID NO: 35.

In certain embodiments, the second polypeptide may comprise a second extracellular region comprising one or more leucine zipper motifs comprising the amino acid sequence set forth in SEQ ID NO: 160 or variant thereof, which may be encoded by the nucleotide sequence set forth in SEQ ID NO: 161; optionally, a second hinge region of a second cytokine receptor chain comprising the amino acid sequence set forth in SEQ ID NO: 64 or variant thereof, which may be encoded by the nucleotide sequence set forth in SEQ ID NO: 65; a second transmembrane region of a second cytokine receptor chain comprising the amino acid sequence set forth in SEQ ID NO: 66 or variant thereof, which may be encoded by the nucleotide sequence set forth in SEQ ID NO: 67; a second intracellular signaling region of a second cytokine receptor chain comprising the amino acid sequence set forth in SEQ ID NO: 69 or variant thereof, which may be encoded by the nucleotide sequence set forth in SEQ ID NO: 69.

In some embodiments, when the chimeric cytokine receptor comprises a first polypeptide comprising the amino acid sequence set forth in SEQ ID NO: 42 or a variant thereof, which may be encoded by the nucleotide sequence set forth in SEQ ID NO: 43, and a second polypeptide comprising the amino acid sequence set forth in SEQ ID NO 72, or variant thereof, which may be encoded by the nucleotide sequence set forth in SEQ ID NO: 73, the one or more leucine zipper motifs from first extracellular region may heterodimerize with the one or more leucine zipper motifs from the second extracellular region. In certain embodiments, the chimeric cytokine receptor comprises a first polypeptide comprising the amino acid sequence set forth in SEQ ID NO: 42 or a variant thereof, and a second polypeptide comprising the amino acid sequence set forth in SEQ ID NO 72, or variant thereof. In certain embodiments, the nucleotide sequence that encodes the first polypeptide of the chimeric cytokine receptor comprises the nucleotide sequence set forth in SEQ ID NO: 43. In certain embodiments, the nucleotide sequence that encodes the second polypeptide of the chimeric cytokine receptor comprises the nucleotide sequence set forth in SEQ ID NO: 73.

In some embodiments, the chimeric cytokine receptor comprises
a) a first polypeptide comprising:
   i) a first extracellular region comprising one or more leucine zipper motifs (e.g., two leucine zipper motifs),
   ii) optionally, a first hinge region derived from the common gamma chain (γc),
   iii) a first transmembrane region derived from the common gamma chain (γc), and
   iv) a first intracellular signaling region of the common gamma chain (γc); and
b) a second polypeptide comprising:
   i) a second extracellular region comprising one or more leucine zipper motifs (e.g., two leucine zipper motifs),
   ii) optionally, a second hinge region derived from the IL-4 receptor alpha chain,
   iii) a second transmembrane region derived from the IL-4 receptor alpha chain, and
   iv) a second intracellular signaling region derived from the IL-4 receptor alpha chain.

In some embodiments, the first extracellular region of the chimeric cytokine receptor comprises the amino acid sequence of SEQ ID NO: 158, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 158. In some embodiments, the nucleotide sequence that encodes the first extracellular region of the chimeric cytokine receptor comprises the nucleotide sequence that encodes the amino acid sequence of SEQ ID NO: 158, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 158. In some embodiments, the nucleotide sequence that encodes the first extracellular region of the chimeric cytokine receptor comprises the nucleotide sequence set forth in SEQ ID NO: 159, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 159. In some embodiments, the first extracellular region of the chimeric cytokine receptor comprises the amino acid sequence set forth in SEQ ID NO: 158. In some embodiments, the nucleotide sequence that encodes the first polypeptide of the chimeric cytokine receptor comprises the nucleotide sequence set forth in SEQ ID NO: 159.

In some embodiments, the first hinge region derived from the common gamma chain (γc) comprises the amino acid sequence of SEQ ID NO: 30, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 30. In some embodiments, the nucleotide sequence that encodes the first hinge region derived from the common gamma chain (γc) comprises the nucleotide sequence that encodes the amino acid sequence of SEQ ID NO: 30, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 30. In some embodiments, the nucleotide sequence that encodes the first hinge region derived from the common gamma chain (γc) comprises the nucleotide sequence set forth in SEQ ID NO: 31, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 31. In some embodiments, the first hinge region derived from the common gamma chain (γc) comprises the amino acid sequence set forth in SEQ ID NO: 30. In some embodiments, the nucleotide sequence that encodes the first hinge region derived from the common gamma chain (γc) comprises the nucleotide sequence set forth in SEQ ID NO: 31.

In some embodiments, the first transmembrane region derived from the common gamma chain (γc) comprises the amino acid sequence of SEQ ID NO: 32, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 32. In some embodiments, the nucleotide sequence that encodes the first transmembrane region derived from the common gamma chain (γc) comprises the nucleotide sequence that encodes the amino acid sequence of SEQ ID NO: 32, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 32. In some embodiments, the nucleotide sequence that encodes the first transmembrane region derived from the common gamma chain (γc) comprises the nucleotide sequence set forth in SEQ ID NO: 33, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 33. In some embodiments, the first transmembrane region derived from the common gamma chain (γc) comprises the amino acid sequence set forth in SEQ ID NO: 32. In some embodiments, the nucleotide sequence that encodes the first transmembrane region derived from the common gamma chain (γc) comprises the nucleotide sequence set forth in SEQ ID NO: 33.

In some embodiments, the first intracellular signaling region derived from the common gamma chain (γc) comprises the amino acid sequence of SEQ ID NO: 34, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 34. In some embodiments, the nucleotide sequence that encodes the first intracellular signaling region derived from the common gamma chain (γc) comprises the nucleotide sequence that encodes the amino acid sequence of SEQ ID NO: 34, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 34. In some embodiments, the nucleotide sequence that encodes the first intracellular signaling region derived from the common gamma chain (γc) comprises the nucleotide sequence set forth in SEQ ID NO: 35, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 35. In some embodiments, the first intracellular signaling region derived from the common gamma chain (γc) comprises the amino acid sequence set forth in SEQ ID NO: 34. In some embodiments, the nucleotide sequence that encodes the first intracellular signaling region derived from the common gamma chain (γc) comprises the nucleotide sequence set forth in SEQ ID NO: 35

In certain embodiment, the second extracellular region of the chimeric cytokine receptor comprises the amino acid sequence of SEQ ID NO: 160, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 160. In certain embodiments, the nucleotide sequence that encodes the second extracellular region of the chimeric cytokine receptor comprises the nucleotide sequence that encodes the amino acid sequence of SEQ ID NO: 160, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 160. In certain embodiments, the nucleotide sequence that encodes the second extracellular region of the chimeric cytokine receptor comprises the nucleotide sequence set forth in SEQ ID NO: 161, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 161. In certain embodiments, the second extracellular region of the chimeric cytokine receptor comprises the amino acid sequence set forth in SEQ ID NO: 160. In certain embodiments, the nucleotide sequence that encodes the second polypeptide of the chimeric cytokine receptor comprises the nucleotide sequence set forth in SEQ ID NO: 161.

In some embodiments, the second hinge region derived from the IL-4 receptor alpha chain comprises the amino acid sequence of SEQ ID NO: 74, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 74. In certain embodiments, the nucleotide sequence that encodes the second hinge region derived from the IL-4 receptor alpha chain comprises the nucleotide sequence that encodes the amino acid sequence of SEQ ID NO: 74, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 74. In certain embodiments, the nucleotide sequence that encodes the second hinge region derived from the IL-4 receptor alpha chain comprises the nucleotide sequence set forth in SEQ ID NO: 75, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 75. In certain embodiments, the second hinge region derived from the IL-4 receptor alpha chain comprises the amino acid sequence set forth in SEQ ID NO: 74. In certain embodiments, the nucleotide sequence that encodes the second hinge region derived from the IL-4 receptor alpha chain comprises the nucleotide sequence set forth in SEQ ID NO: 75.

In some embodiments, the second transmembrane region derived from the IL-4 receptor alpha chain comprises the amino acid sequence of SEQ ID NO: 76, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 76. In certain embodiments, the nucleotide sequence that encodes the second transmembrane region derived from the IL-4 receptor alpha chain comprises the nucleotide sequence that encodes the amino acid sequence of SEQ ID NO: 76, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 76. In certain embodiments, the nucleotide sequence that encodes the second transmembrane region derived from the IL-4 receptor alpha chain comprises the nucleotide sequence set forth in SEQ ID NO: 77, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 77. In certain embodiments, the second transmembrane region derived from the IL-4 receptor alpha chain comprises the amino acid sequence set forth in SEQ ID NO: 76. In certain embodiments, the nucleotide sequence that encodes the second transmembrane region derived from the TL-4 receptor alpha chain comprises the nucleotide sequence set forth in SEQ ID NO: 77.

In some embodiments, the second intracellular signaling region derived from the IL-4 receptor alpha chain comprises the amino acid sequence of SEQ ID NO: 78, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 78. In certain embodiments, the nucleotide sequence that encodes the second intracellular signaling region derived from the IL-4 receptor alpha chain comprises the nucleotide sequence that encodes the amino acid sequence of SEQ ID NO: 78, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 78. In certain embodiments, the nucleotide sequence that encodes the second intracellular signaling region derived from the IL-4 receptor alpha chain comprises the nucleotide sequence set forth in SEQ ID NO: 79, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 79. In certain embodiments, the second intracellular signaling region derived from the IL-4 receptor alpha chain comprises the amino acid sequence set forth in SEQ ID NO: 78. In certain embodiments, the nucleotide sequence that encodes the second intracellular signaling region derived from the IL-4 receptor alpha chain comprises the nucleotide sequence set forth in SEQ ID NO: 79.

In some embodiments, the chimeric cytokine receptor comprises a first polypeptide comprising the amino acid set for in SEQ ID NO: 42, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 42. In certain embodiments, the nucleotide sequence that encodes the first polypeptide of the chimeric cytokine receptor comprises the nucleotide sequence that encodes the amino acid sequence of SEQ ID NO: 42, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 42. In certain embodiments, the nucleotide sequence that encodes the first polypeptide of the chimeric cytokine receptor comprises the nucleotide sequence set forth in SEQ ID NO: 43, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 43. In certain embodiments, the first polypeptide of the chimeric cytokine receptor comprises the amino acid sequence set forth in SEQ ID NO: 42. In certain embodiments, the nucleotide sequence that encodes the first polypeptide of the chimeric cytokine receptor comprises the nucleotide sequence set forth in SEQ ID NO: 43.

In some embodiments, the chimeric cytokine receptor comprises a second polypeptide comprising the amino acid set for in SEQ ID NO: 82, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 82. In certain embodiments, the nucleotide sequence that encodes the second polypeptide of the chimeric cytokine receptor comprises the nucleotide sequence that encodes the amino acid sequence of SEQ ID NO: 82, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 82. In certain embodiments, the nucleotide sequence that encodes the second polypeptide of the chimeric cytokine receptor comprises the nucleotide sequence set forth in SEQ ID NO: 83, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 83. In certain embodiments, the second polypeptide of the chimeric cytokine receptor comprises the amino acid sequence set forth in SEQ ID NO: 82. In certain embodiments, the nucleotide sequence that encodes the second polypeptide of the chimeric cytokine receptor comprises the nucleotide sequence set forth in SEQ ID NO: 83.

In certain embodiments, the first polypeptide may comprise a first extracellular region comprising one or more leucine zipper motifs comprising the amino acid sequence set forth in SEQ ID NO: 158 or variant thereof, which may be encoded by the nucleotide sequence set forth in SEQ ID NO: 159; optionally, a first hinge region of a first cytokine receptor chain comprising the amino acid sequence set forth in SEQ ID NO: 30 or variant thereof, which may be encoded by the nucleotide sequence set forth in SEQ ID NO: 31; a first transmembrane region of a first cytokine receptor chain comprising the amino acid sequence set forth in SEQ ID NO: 32 or variant thereof, which may be encoded by the nucleotide sequence set forth in SEQ ID NO: 33; a first intracellular signaling region of a first cytokine receptor chain comprising the amino acid sequence set forth in SEQ ID NO: 34 or variant thereof, which may be encoded by the nucleotide sequence set forth in SEQ ID NO: 35.

In certain embodiments, the second polypeptide may comprise a second extracellular region comprising one or more leucine zipper motifs comprising the amino acid sequence set forth in SEQ ID NO: 160 or variant thereof, which may be encoded by the nucleotide sequence set forth in SEQ ID NO: 161; optionally, a second hinge region of a second cytokine receptor chain comprising the amino acid sequence set forth in SEQ ID NO: 74 or variant thereof, which may be encoded by the nucleotide sequence set forth in SEQ ID NO: 75; a second transmembrane region of a second cytokine receptor chain comprising the amino acid sequence set forth in SEQ ID NO: 76 or variant thereof, which may be encoded by the nucleotide sequence set forth in SEQ ID NO: 77; a second intracellular signaling region of a second cytokine receptor chain comprising the amino acid sequence set forth in SEQ ID NO: 78 or variant thereof, which may be encoded by the nucleotide sequence set forth in SEQ ID NO: 79.

In some embodiments, when the chimeric cytokine receptor comprises a first polypeptide comprising the amino acid sequence set forth in SEQ ID NO: 42 or a variant thereof, which may be encoded by the nucleotide sequence set forth in SEQ ID NO: 43, and a second polypeptide comprising the amino acid sequence set forth in SEQ ID NO 82, or variant thereof, which may be encoded by the nucleotide sequence set forth in SEQ ID NO: 83, the one or more leucine zipper motifs from first extracellular region may heterodimerize with the one or more leucine zipper motifs from the second extracellular region. In certain embodiments, the chimeric cytokine receptor comprises a first polypeptide comprising the amino acid sequence set forth in SEQ ID NO: 42 or a variant thereof, and a second polypeptide comprising the amino acid sequence set forth in SEQ ID NO 82, or variant thereof. In certain embodiments, the nucleotide sequence that encodes the first polypeptide of the chimeric cytokine receptor comprises the nucleotide sequence set forth in SEQ ID NO: 43. In certain embodiments, the nucleotide sequence that encodes the second polypeptide of the chimeric cytokine receptor comprises the nucleotide sequence set forth in SEQ ID NO: 83.

In some embodiments, the chimeric cytokine receptor comprises
a) a first polypeptide comprising:
   i) a first extracellular region comprising one or more leucine zipper motifs (e.g., two leucine zipper motifs),
   ii) optionally, a first hinge region derived from the IL-12 receptor beta 1 chain,
   iii) a first transmembrane region derived from the IL-12 receptor beta 1 chain, and
   iv) a first intracellular signaling region of the IL-12 receptor beta 1 chain; and
b) a second polypeptide comprising:
   i) a second extracellular region comprising one or more leucine zipper motifs (e.g., two leucine zipper motifs),
   ii) optionally, a second hinge region derived from the IL-12 receptor beta 2 chain,
   iii) a second transmembrane region derived from the IL-12 receptor beta 2 chain, and
   iv) a second intracellular signaling region derived from the IL-12 receptor beta 2 chain.

In some embodiments, the first extracellular region of the chimeric cytokine receptor comprises the amino acid sequence of SEQ ID NO: 158, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 158. In some embodiments, the nucleotide sequence that encodes the first extracellular region of the chimeric cytokine receptor comprises the nucleotide sequence that encodes the amino acid sequence of SEQ ID NO: 158, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 158. In some embodiments, the nucleotide sequence that encodes the first extracellular region of the chimeric cytokine receptor comprises the nucleotide sequence set forth in SEQ ID NO: 159, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 159. In some embodiments, the first extracellular region of the chimeric cytokine receptor comprises the amino acid sequence set forth in SEQ ID NO: 158. In some embodiments, the nucleotide sequence that encodes the first polypeptide of the chimeric cytokine receptor comprises the nucleotide sequence set forth in SEQ ID NO: 159.

In some embodiments, the first hinge region derived from the IL-12 receptor beta 1 chain comprises the amino acid sequence of SEQ ID NO: 84, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 84. In some embodiments, the nucleotide sequence that encodes the first hinge region derived from the IL-12 receptor beta 1 chain comprises the nucleotide sequence that encodes the amino acid sequence of SEQ ID NO: 84, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 84. In some embodiments, the nucleotide sequence that encodes the first hinge region derived from the IL-12 receptor beta 1 chain comprises the nucleotide sequence set forth in SEQ ID NO: 85, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 85. In some embodiments, the first hinge region derived from the IL-12 receptor beta 1 chain comprises the amino acid sequence set forth in SEQ ID NO: 84. In some embodiments, the nucleotide sequence that encodes the first hinge region derived from the IL-12 receptor beta 1 chain comprises the nucleotide sequence set forth in SEQ ID NO: 85.

In some embodiments, the first transmembrane region derived from the IL-12 receptor beta 1 chain comprises the amino acid sequence of SEQ ID NO: 86, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 86. In some embodiments, the nucleotide sequence that encodes the first transmembrane region derived from the IL-12 receptor beta 1 chain comprises the nucleotide sequence that encodes the amino acid sequence of SEQ ID NO: 86, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 86. In some embodiments, the nucleotide sequence that encodes the first transmembrane region derived from the IL-12 receptor beta 1 chain comprises the nucleotide sequence set forth in SEQ ID NO: 87, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 87. In some embodiments, the first transmembrane region derived from the IL-12 receptor beta 1 chain comprises the amino acid sequence set forth in SEQ ID NO: 86. In some embodiments, the nucleotide sequence that encodes the first transmembrane region derived from the IL-12 receptor beta 1 chain comprises the nucleotide sequence set forth in SEQ ID NO: 87.

In some embodiments, the first intracellular signaling region derived from the IL-12 receptor beta 1 chain comprises the amino acid sequence of SEQ ID NO: 88, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 88. In some embodiments, the nucleotide sequence that encodes the first intracellular signaling region derived from the IL-12 receptor beta 1 chain comprises the nucleotide sequence that encodes the amino acid sequence of SEQ ID NO: 88, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 88. In some embodiments, the nucleotide sequence that encodes the first intracellular signaling region derived from the IL-12 receptor beta 1 chain comprises the nucleotide sequence set forth in SEQ ID NO: 89, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 89. In some embodiments, the first intracellular signaling region derived from the IL-12 receptor beta 1 chain comprises the amino acid sequence set forth in SEQ ID NO: 88. In some embodiments, the nucleotide sequence that encodes the first intracellular signaling region derived from the IL-12 receptor beta 1 chain comprises the nucleotide sequence set forth in SEQ ID NO: 89.

In certain embodiment, the second extracellular region of the chimeric cytokine receptor comprises the amino acid sequence of SEQ ID NO: 160, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 160. In certain embodiments, the nucleotide sequence that encodes the second extracellular region of the chimeric cytokine receptor comprises the nucleotide sequence that encodes the amino acid sequence of SEQ ID NO: 160, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 160. In certain embodiments, the nucleotide sequence that encodes the second extracellular region of the chimeric cytokine receptor comprises the nucleotide sequence set forth in SEQ ID NO: 161, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 161. In certain embodiments, the second extracellular region of the chimeric cytokine receptor comprises the amino acid sequence set forth in SEQ ID NO: 160. In certain embodiments, the nucleotide sequence that encodes the second polypeptide of the chimeric cytokine receptor comprises the nucleotide sequence set forth in SEQ ID NO: 161.

In some embodiments, the second hinge region derived from the IL-12 receptor beta 2 chain comprises the amino acid sequence of SEQ ID NO: 94, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 94. In certain embodiments, the nucleotide sequence that encodes the second hinge region derived from the IL-12 receptor beta 2 chain comprises the nucleotide sequence that encodes the amino acid sequence of SEQ ID NO: 94, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 94. In certain embodiments, the nucleotide sequence that encodes the second hinge region derived from the IL-12 receptor beta 2 chain comprises the nucleotide sequence set forth in SEQ ID NO: 95, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 95. In certain embodiments, the second hinge region derived from the IL-12 receptor beta 2 chain comprises the amino acid sequence set forth in SEQ ID NO: 94. In certain embodiments, the nucleotide sequence that encodes the second hinge region derived from the IL-12 receptor beta 2 chain comprises the nucleotide sequence set forth in SEQ ID NO: 95.

In some embodiments, the second transmembrane region derived from the IL-12 receptor beta 2 chain comprises the amino acid sequence of SEQ ID NO: 96, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 96. In certain embodiments, the nucleotide sequence that encodes the second transmembrane region derived from the IL-12 receptor beta 2 chain comprises the nucleotide sequence that encodes the amino acid sequence of SEQ ID NO: 96, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 96. In certain embodiments, the nucleotide sequence that encodes the second transmembrane region derived from the IL-12 receptor beta 2 chain comprises the nucleotide sequence set forth in SEQ ID NO: 97, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 97. In certain embodiments, the second transmembrane region derived from the IL-12 receptor beta 2 chain comprises the amino acid sequence set forth in SEQ ID NO: 96. In certain embodiments, the nucleotide sequence that encodes the second transmembrane region derived from the IL-12 receptor beta 2 chain comprises the nucleotide sequence set forth in SEQ ID NO: 97.

In some embodiments, the second intracellular signaling region derived from the IL-12 receptor beta 2 chain comprises the amino acid sequence of SEQ ID NO: 98, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 98. In certain embodiments, the nucleotide sequence that encodes the second intracellular signaling region derived from the IL-12 receptor beta 2 chain comprises the nucleotide sequence that encodes the amino acid sequence of SEQ ID NO: 98, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 98. In certain embodiments, the nucleotide sequence that encodes the second intracellular signaling region derived from the IL-12 receptor beta 2 chain comprises the nucleotide sequence set forth in SEQ ID NO: 99, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 99. In certain embodiments, the second intracellular signaling region derived from the IL-12 receptor beta 2 chain comprises the amino acid sequence set forth in SEQ ID NO: 98. In certain embodiments, the nucleotide sequence that encodes the second intracellular signaling region derived from the IL-12 receptor beta 2 chain comprises the nucleotide sequence set forth in SEQ ID NO: 99.

In some embodiments, the chimeric cytokine receptor comprises a first polypeptide comprising the amino acid set for in SEQ ID NO: 92, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 92. In certain embodiments, the nucleotide sequence that encodes the first polypeptide of the chimeric cytokine receptor comprises the nucleotide sequence that encodes the amino acid sequence of SEQ ID NO: 92, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 92. In certain embodiments, the nucleotide sequence that encodes the first polypeptide of the chimeric cytokine receptor comprises the nucleotide sequence set forth in SEQ ID NO: 93, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 93. In certain embodiments, the first polypeptide of the chimeric cytokine receptor comprises the amino acid sequence set forth in SEQ ID NO: 92. In certain embodiments, the nucleotide sequence that encodes the first polypeptide of the chimeric cytokine receptor comprises the nucleotide sequence set forth in SEQ ID NO: 93.

In some embodiments, the chimeric cytokine receptor comprises a second polypeptide comprising the amino acid set for in SEQ ID NO: 102, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 102. In certain embodiments, the nucleotide sequence that encodes the second polypeptide of the chimeric cytokine receptor comprises the nucleotide sequence that encodes the amino acid sequence of SEQ ID NO: 102, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 102. In certain embodiments, the nucleotide sequence that encodes the second polypeptide of the chimeric cytokine receptor comprises the nucleotide sequence set forth in SEQ ID NO: 103, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 103. In certain embodiments, the second polypeptide of the chimeric cytokine receptor comprises the amino acid sequence set forth in SEQ ID NO: 102. In certain embodiments, the nucleotide sequence that encodes the second polypeptide of the chimeric cytokine receptor comprises the nucleotide sequence set forth in SEQ ID NO: 103.

In certain embodiments, the first polypeptide may comprise a first extracellular region comprising one or more leucine zipper motifs comprising the amino acid sequence set forth in SEQ ID NO: 158 or variant thereof, which may be encoded by the nucleotide sequence set forth in SEQ ID NO: 159; optionally, a first hinge region of a first cytokine receptor chain comprising the amino acid sequence set forth in SEQ ID NO: 84 or variant thereof, which may be encoded by the nucleotide sequence set forth in SEQ ID NO: 85; a first transmembrane region of a first cytokine receptor chain comprising the amino acid sequence set forth in SEQ ID NO: 86 or variant thereof, which may be encoded by the nucleotide sequence set forth in SEQ ID NO: 87; a first intracellular signaling region of a first cytokine receptor chain comprising the amino acid sequence set forth in SEQ ID NO: 88 or variant thereof, which may be encoded by the nucleotide sequence set forth in SEQ ID NO: 89.

In certain embodiments, the second polypeptide may comprise a second extracellular region comprising one or more leucine zipper motifs comprising the amino acid sequence set forth in SEQ ID NO: 160 or variant thereof, which may be encoded by the nucleotide sequence set forth in SEQ ID NO: 161; optionally, a second hinge region of a second cytokine receptor chain comprising the amino acid sequence set forth in SEQ ID NO: 94 or variant thereof, which may be encoded by the nucleotide sequence set forth in SEQ ID NO: 95; a second transmembrane region of a second cytokine receptor chain comprising the amino acid sequence set forth in SEQ ID NO: 96 or variant thereof, which may be encoded by the nucleotide sequence set forth in SEQ ID NO: 97; a second intracellular signaling region of a second cytokine receptor chain comprising the amino acid sequence set forth in SEQ ID NO: 98 or variant thereof, which may be encoded by the nucleotide sequence set forth in SEQ ID NO: 99.

In some embodiments, when the chimeric cytokine receptor comprises a first polypeptide comprising the amino acid sequence set forth in SEQ ID NO: 92 or a variant thereof, which may be encoded by the nucleotide sequence set forth in SEQ ID NO: 93, and a second polypeptide comprising the amino acid sequence set forth in SEQ ID NO 102, or variant thereof, which may be encoded by the nucleotide sequence set forth in SEQ ID NO: 103, the one or more leucine zipper motifs from first extracellular region may heterodimerize with the one or more leucine zipper motifs from the second extracellular region. In certain embodiments, the chimeric cytokine receptor comprises a first polypeptide comprising the amino acid sequence set forth in SEQ ID NO: 92 or a variant thereof, and a second polypeptide comprising the amino acid sequence set forth in SEQ ID NO 102, or variant thereof. In certain embodiments, the nucleotide sequence that encodes the first polypeptide of the chimeric cytokine receptor comprises the nucleotide sequence set forth in SEQ ID NO: 93. In certain embodiments, the nucleotide sequence that encodes the second polypeptide of the chimeric cytokine receptor comprises the nucleotide sequence set forth in SEQ ID NO: 103.

In some embodiments, the chimeric cytokine receptor comprises
a) a first polypeptide comprising:
   i) a first extracellular region comprising one or more leucine zipper motifs (e.g., two leucine zipper motifs),
   ii) optionally, a first hinge region derived from the IL-23 receptor chain,
   iii) a first transmembrane region derived from the IL-23 receptor chain, and
   iv) a first intracellular signaling region of the IL-23 receptor chain; and
b) a second polypeptide comprising:
   i) a second extracellular region comprising one or more leucine zipper motifs (e.g., two leucine zipper motifs),
   ii) optionally, a second hinge region derived from the IL-12 receptor beta 2 chain,
   iii) a second transmembrane region derived from the IL-12 receptor beta 2 chain, and
   iv) a second intracellular signaling region derived from the IL-12 receptor beta 2 chain.

In some embodiments, the first extracellular region of the chimeric cytokine receptor comprises the amino acid sequence of SEQ ID NO: 158, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 158. In some embodiments, the nucleotide sequence that encodes the first extracellular region of the chimeric cytokine receptor comprises the nucleotide sequence that encodes the amino acid sequence of SEQ ID NO: 158, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 158. In some embodiments, the nucleotide sequence that encodes the first extracellular region of the chimeric cytokine receptor comprises the nucleotide sequence set forth in SEQ ID NO: 159, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 159. In some embodiments, the first extracellular region of the chimeric cytokine receptor comprises the amino acid sequence set forth in SEQ ID NO: 158. In some embodiments, the nucleotide sequence that encodes the first polypeptide of the chimeric cytokine receptor comprises the nucleotide sequence set forth in SEQ ID NO: 159

In some embodiments, the first hinge region derived from the IL-23 receptor chain comprises the amino acid sequence of SEQ ID NO: 104, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 104. In some embodiments, the nucleotide sequence that encodes the first hinge region derived from the IL-23 receptor chain comprises the nucleotide sequence that encodes the amino acid sequence of SEQ ID NO: 104, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 104. In some embodiments, the nucleotide sequence that encodes the first hinge region derived from the IL-23 receptor chain comprises the nucleotide sequence set forth in SEQ ID NO: 105, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 105. In some embodiments, the first hinge region derived from the IL-23 receptor chain comprises the amino acid sequence set forth in SEQ ID NO: 104. In some embodiments, the nucleotide sequence that encodes the first hinge region derived from the IL-23 receptor chain comprises the nucleotide sequence set forth in SEQ ID NO: 105.

In some embodiments, the first transmembrane region derived from the IL-23 receptor chain comprises the amino acid sequence of SEQ ID NO: 106, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 106. In some embodiments, the nucleotide sequence that encodes the first transmembrane region derived from the IL-23 receptor chain comprises the nucleotide sequence that encodes the amino acid sequence of SEQ ID NO: 106, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 106. In some embodiments, the nucleotide sequence that encodes the first transmembrane region derived from the IL-23 receptor chain comprises the nucleotide sequence set forth in SEQ ID NO: 107, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 107. In some embodiments, the first transmembrane region derived from the IL-23 receptor chain comprises the amino acid sequence set forth in SEQ ID NO: 106. In some embodiments, the nucleotide sequence that encodes the first transmembrane region derived from the IL-23 receptor chain comprises the nucleotide sequence set forth in SEQ ID NO: 107.

In some embodiments, the first intracellular signaling region derived from the IL-23 receptor chain comprises the amino acid sequence of SEQ ID NO: 108, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 108. In some embodiments, the nucleotide sequence that encodes the first intracellular signaling region derived from the IL-23 receptor chain comprises the nucleotide sequence that encodes the amino acid sequence of SEQ ID NO: 108, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 108. In some embodiments, the nucleotide sequence that encodes the first intracellular signaling region derived from the TL-23 receptor chain comprises the nucleotide sequence set forth in SEQ ID NO: 109, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 109. In some embodiments, the first intracellular signaling region derived from the TL-23 receptor chain comprises the amino acid sequence set forth in SEQ ID NO: 108. In some embodiments, the nucleotide sequence that encodes the first intracellular signaling region derived from the IL-23 receptor chain comprises the nucleotide sequence set forth in SEQ ID NO: 109.

In certain embodiment, the second extracellular region of the chimeric cytokine receptor comprises the amino acid sequence of SEQ ID NO: 160, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 160. In certain embodiments, the nucleotide sequence that encodes the second extracellular region of the chimeric cytokine receptor comprises the nucleotide sequence that encodes the amino acid sequence of SEQ ID NO: 160, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 160. In certain embodiments, the nucleotide sequence that encodes the second extracellular region of the chimeric cytokine receptor comprises the nucleotide sequence set forth in SEQ ID NO: 161, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 161. In certain embodiments, the second extracellular region of the chimeric cytokine receptor comprises the amino acid sequence set forth in SEQ ID NO: 160. In certain embodiments, the nucleotide sequence that encodes the second polypeptide of the chimeric cytokine receptor comprises the nucleotide sequence set forth in SEQ ID NO: 161.

In some embodiments, the second hinge region derived from the IL-12 receptor beta 2 chain comprises the amino acid sequence of SEQ ID NO: 94, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 94. In certain embodiments, the nucleotide sequence that encodes the second hinge region derived from the IL-12 receptor beta 2 chain comprises the nucleotide sequence that encodes the amino acid sequence of SEQ ID NO: 94, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 94. In certain embodiments, the nucleotide sequence that encodes the second hinge region derived from the IL-12 receptor beta 2 chain comprises the nucleotide sequence set forth in SEQ ID NO: 95, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 95. In certain embodiments, the second hinge region derived from the IL-12 receptor beta 2 chain comprises the amino acid sequence set forth in SEQ ID NO: 94. In certain embodiments, the nucleotide sequence that encodes the second hinge region derived from the IL-12 receptor beta 2 chain comprises the nucleotide sequence set forth in SEQ ID NO: 95.

In some embodiments, the second transmembrane region derived from the IL-12 receptor beta 2 chain comprises the amino acid sequence of SEQ ID NO: 96, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 96. In certain embodiments, the nucleotide sequence that encodes the second transmembrane region derived from the IL-12 receptor beta 2 chain comprises the nucleotide sequence that encodes the amino acid sequence of SEQ ID NO: 96, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 96. In certain embodiments, the nucleotide sequence that encodes the second transmembrane region derived from the IL-12 receptor beta 2 chain comprises the nucleotide sequence set forth in SEQ ID NO: 97, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 97. In certain embodiments, the second transmembrane region derived from the IL-12 receptor beta 2 chain comprises the amino acid sequence set forth in SEQ ID NO: 96. In certain embodiments, the nucleotide sequence that encodes the second transmembrane region derived from the IL-12 receptor beta 2 chain comprises the nucleotide sequence set forth in SEQ ID NO: 97.

In some embodiments, the second intracellular signaling region derived from the IL-12 receptor beta 2 chain comprises the amino acid sequence of SEQ ID NO: 98, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 98. In certain embodiments, the nucleotide sequence that encodes the second intracellular signaling region derived from the IL-12 receptor beta 2 chain comprises the nucleotide sequence that encodes the amino acid sequence of SEQ ID NO: 98, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 98. In certain embodiments, the nucleotide sequence that encodes the second intracellular signaling region derived from the IL-12 receptor beta 2 chain comprises the nucleotide sequence set forth in SEQ ID NO: 99, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 99. In certain embodiments, the second intracellular signaling region derived from the IL-12 receptor beta 2 chain comprises the amino acid sequence set forth in SEQ ID NO: 98. In certain embodiments, the nucleotide sequence that encodes the second intracellular signaling region derived from the IL-12 receptor beta 2 chain comprises the nucleotide sequence set forth in SEQ ID NO: 99.

In some embodiments, the chimeric cytokine receptor comprises a first polypeptide comprising the amino acid set for in SEQ ID NO: 112, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 112. In certain embodiments, the nucleotide sequence that encodes the first polypeptide of the chimeric cytokine receptor comprises the nucleotide sequence that encodes the amino acid sequence of SEQ ID NO: 112, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 112. In certain embodiments, the nucleotide sequence that encodes the first polypeptide of the chimeric cytokine receptor comprises the nucleotide sequence set forth in SEQ ID NO: 113, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 113. In certain embodiments, the first polypeptide of the chimeric cytokine receptor comprises the amino acid sequence set forth in SEQ ID NO: 112. In certain embodiments, the nucleotide sequence that encodes the first polypeptide of the chimeric cytokine receptor comprises the nucleotide sequence set forth in SEQ ID NO: 113.

In some embodiments, the chimeric cytokine receptor comprises a second polypeptide comprising the amino acid set for in SEQ ID NO: 102, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 102. In certain embodiments, the nucleotide sequence that encodes the second polypeptide of the chimeric cytokine receptor comprises the nucleotide sequence that encodes the amino acid sequence of SEQ ID NO: 102, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 102. In certain embodiments, the nucleotide sequence that encodes the second polypeptide of the chimeric cytokine receptor comprises the nucleotide sequence set forth in SEQ ID NO: 103, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 103. In certain embodiments, the second polypeptide of the chimeric cytokine receptor comprises the amino acid sequence set forth in SEQ ID NO: 102. In certain embodiments, the nucleotide sequence that encodes the second polypeptide of the chimeric cytokine receptor comprises the nucleotide sequence set forth in SEQ ID NO: 103.

In certain embodiments, the first polypeptide may comprise a first extracellular region comprising one or more leucine zipper motifs comprising the amino acid sequence set forth in SEQ ID NO: 158 or variant thereof, which may be encoded by the nucleotide sequence set forth in SEQ ID NO: 159; optionally, a first hinge region of a first cytokine receptor chain comprising the amino acid sequence set forth in SEQ ID NO: 104 or variant thereof, which may be encoded by the nucleotide sequence set forth in SEQ ID NO: 105; a first transmembrane region of a first cytokine receptor chain comprising the amino acid sequence set forth in SEQ ID NO: 106 or variant thereof, which may be encoded by the nucleotide sequence set forth in SEQ ID NO: 107; a first intracellular signaling region of a first cytokine receptor chain comprising the amino acid sequence set forth in SEQ ID NO: 108 or variant thereof, which may be encoded by the nucleotide sequence set forth in SEQ ID NO: 109.

In certain embodiments, the second polypeptide may comprise a second extracellular region comprising one or more leucine zipper motifs comprising the amino acid sequence set forth in SEQ ID NO: 160 or variant thereof, which may be encoded by the nucleotide sequence set forth in SEQ ID NO: 161; optionally, a second hinge region of a second cytokine receptor chain comprising the amino acid sequence set forth in SEQ ID NO: 94 or variant thereof, which may be encoded by the nucleotide sequence set forth in SEQ ID NO: 95; a second transmembrane region of a second cytokine receptor chain comprising the amino acid sequence set forth in SEQ ID NO: 96 or variant thereof, which may be encoded by the nucleotide sequence set forth in SEQ ID NO: 97; a second intracellular signaling region of a second cytokine receptor chain comprising the amino acid sequence set forth in SEQ ID NO: 98 or variant thereof, which may be encoded by the nucleotide sequence set forth in SEQ ID NO: 99.

In some embodiments, when the chimeric cytokine receptor comprises a first polypeptide comprising the amino acid sequence set forth in SEQ ID NO: 112 or a variant thereof, which may be encoded by the nucleotide sequence set forth in SEQ ID NO: 113, and a second polypeptide comprising the amino acid sequence set forth in SEQ ID NO 102, or variant thereof, which may be encoded by the nucleotide sequence set forth in SEQ ID NO: 103, the one or more leucine zipper motifs from first extracellular region may heterodimerize with the one or more leucine zipper motifs from the second extracellular region. In certain embodiments, the chimeric cytokine receptor comprises a first polypeptide comprising the amino acid sequence set forth in SEQ ID NO: 112 or a variant thereof, and a second polypeptide comprising the amino acid sequence set forth in SEQ ID NO 103, or variant thereof. In certain embodiments, the nucleotide sequence that encodes the first polypeptide of the chimeric cytokine receptor comprises the nucleotide sequence set forth in SEQ ID NO: 113. In certain embodiments, the nucleotide sequence that encodes the second polypeptide of the chimeric cytokine receptor comprises the nucleotide sequence set forth in SEQ ID NO: 103.

In some embodiments, the chimeric cytokine receptor comprises
a) a first polypeptide comprising:
   i) a first extracellular region comprising one or more leucine zipper motifs (e.g., two leucine zipper motifs),
   ii) optionally, a first hinge region derived from the IL-10 receptor alpha chain,
   iii) a first transmembrane region derived from the IL-10 receptor alpha chain, and
   iv) a first intracellular signaling region of the IL-10 receptor alpha chain; and
b) a second polypeptide comprising:
   i) a second extracellular region comprising one or more leucine zipper motifs (e.g., two leucine zipper motifs),
   ii) optionally, a second hinge region derived from the IL-10 receptor beta chain,
   iii) a second transmembrane region derived from the IL-10 receptor beta chain, and
   iv) a second intracellular signaling region derived from the IL-10 receptor beta chain.

In some embodiments, the first extracellular region of the chimeric cytokine receptor comprises the amino acid sequence of SEQ ID NO: 158, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 158. In some embodiments, the nucleotide sequence that encodes the first extracellular region of the chimeric cytokine receptor comprises the nucleotide sequence that encodes the amino acid sequence of SEQ ID NO: 158, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 158. In some embodiments, the nucleotide sequence that encodes the first extracellular region of the chimeric cytokine receptor comprises the nucleotide sequence set forth in SEQ ID NO: 159, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 159. In some embodiments, the first extracellular region of the chimeric cytokine receptor comprises the amino acid sequence set forth in SEQ ID NO: 158. In some embodiments, the nucleotide sequence that encodes the first polypeptide of the chimeric cytokine receptor comprises the nucleotide sequence set forth in SEQ ID NO: 159.

In some embodiments, the first hinge region derived from the IL-10 receptor alpha chain comprises the amino acid sequence of SEQ ID NO: 114, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 114. In some embodiments, the nucleotide sequence that encodes the first hinge region derived from the IL-10 receptor alpha chain comprises the nucleotide sequence that encodes the amino acid sequence of SEQ ID NO: 114, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 114. In some embodiments, the nucleotide sequence that encodes the first hinge region derived from the IL-10 receptor alpha chain comprises the nucleotide sequence set forth in SEQ ID NO: 115, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 115. In some embodiments, the first hinge region derived from the IL-10 receptor alpha chain comprises the amino acid sequence set forth in SEQ ID NO: 114. In some embodiments, the nucleotide sequence that encodes the first hinge region derived from the IL-10 receptor alpha chain comprises the nucleotide sequence set forth in SEQ ID NO: 115.

In some embodiments, the first transmembrane region derived from the IL-10 receptor alpha chain comprises the amino acid sequence of SEQ ID NO: 116, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 116. In some embodiments, the nucleotide sequence that encodes the first transmembrane region derived from the IL-10 receptor alpha chain comprises the nucleotide sequence that encodes the amino acid sequence of SEQ ID NO: 116, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 116. In some embodiments, the nucleotide sequence that encodes the first transmembrane region derived from the IL-10 receptor alpha chain comprises the nucleotide sequence set forth in SEQ ID NO: 117, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 117. In some embodiments, the first transmembrane region derived from the IL-10 receptor alpha chain comprises the amino acid sequence set forth in SEQ ID NO: 116. In some embodiments, the nucleotide sequence that encodes the first transmembrane region derived from the IL-10 receptor alpha chain comprises the nucleotide sequence set forth in SEQ ID NO: 117.

In some embodiments, the first intracellular signaling region derived from the IL-10 receptor alpha chain comprises the amino acid sequence of SEQ ID NO: 118, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 118. In some embodiments, the nucleotide sequence that encodes the first intracellular signaling region derived from the IL-10 receptor alpha chain comprises the nucleotide sequence that encodes the amino acid sequence of SEQ ID NO: 118, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 118. In some embodiments, the nucleotide sequence that encodes the first intracellular signaling region derived from the IL-10 receptor alpha chain comprises the nucleotide sequence set forth in SEQ ID NO: 119, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 119. In some embodiments, the first intracellular signaling region derived from the IL-10 receptor alpha chain comprises the amino acid sequence set forth in SEQ ID NO: 118. In some embodiments, the nucleotide sequence that encodes the first intracellular signaling region derived from the IL-10 receptor alpha chain comprises the nucleotide sequence set forth in SEQ ID NO: 119.

In certain embodiment, the second extracellular region of the chimeric cytokine receptor comprises the amino acid sequence of SEQ ID NO: 160, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 160. In certain embodiments, the nucleotide sequence that encodes the second extracellular region of the chimeric cytokine receptor comprises the nucleotide sequence that encodes the amino acid sequence of SEQ ID NO: 160, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 160. In certain embodiments, the nucleotide sequence that encodes the second extracellular region of the chimeric cytokine receptor comprises the nucleotide sequence set forth in SEQ ID NO: 161, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 161. In certain embodiments, the second extracellular region of the chimeric cytokine receptor comprises the amino acid sequence set forth in SEQ ID NO: 160. In certain embodiments, the nucleotide sequence that encodes the second polypeptide of the chimeric cytokine receptor comprises the nucleotide sequence set forth in SEQ ID NO: 161.

In some embodiments, the second hinge region derived from the IL-10 receptor beta chain comprises the amino acid sequence of SEQ ID NO: 124, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 124. In certain embodiments, the nucleotide sequence that encodes the second hinge region derived from the IL-10 receptor beta chain comprises the nucleotide sequence that encodes the amino acid sequence of SEQ ID NO: 124, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 124. In certain embodiments, the nucleotide sequence that encodes the second hinge region derived from the IL-10 receptor beta chain comprises the nucleotide sequence set forth in SEQ ID NO: 125, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 125. In certain embodiments, the second hinge region derived from the IL-10 receptor beta chain comprises the amino acid sequence set forth in SEQ ID NO: 124. In certain embodiments, the nucleotide sequence that encodes the second hinge region derived from the IL-10 receptor beta chain comprises the nucleotide sequence set forth in SEQ ID NO: 125.

In some embodiments, the second transmembrane region derived from the IL-10 receptor beta chain comprises the amino acid sequence of SEQ ID NO: 126, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 126. In certain embodiments, the nucleotide sequence that encodes the second transmembrane region derived from the IL-10 receptor beta chain comprises the nucleotide sequence that encodes the amino acid sequence of SEQ ID NO: 126, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 126. In certain embodiments, the nucleotide sequence that encodes the second transmembrane region derived from the IL-10 receptor beta chain comprises the nucleotide sequence set forth in SEQ ID NO: 127, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 127. In certain embodiments, the second transmembrane region derived from the IL-10 receptor beta chain comprises the amino acid sequence set forth in SEQ ID NO: 126. In certain embodiments, the nucleotide sequence that encodes the second transmembrane region derived from the IL-10 receptor beta chain comprises s the nucleotide sequence set forth in SEQ ID NO: 127.

In some embodiments, the second intracellular signaling region derived from the IL-10 receptor beta chain comprises the amino acid sequence of SEQ ID NO: 128, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 128. In certain embodiments, the nucleotide sequence that encodes the second intracellular signaling region derived from the IL-10 receptor beta chain comprises the nucleotide sequence that encodes the amino acid sequence of SEQ ID NO: 128, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 128. In certain embodiments, the nucleotide sequence that encodes the second intracellular signaling region derived from the IL-10 receptor beta chain comprises the nucleotide sequence set forth in SEQ ID NO: 129, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 129. In certain embodiments, the second intracellular signaling region derived from the IL-10 receptor beta chain comprises the amino acid sequence set forth in SEQ ID NO: 128. In certain embodiments, the nucleotide sequence that encodes the second intracellular signaling region derived from the IL-10 receptor beta chain comprises the nucleotide sequence set forth in SEQ ID NO: 129.

In some embodiments, the chimeric cytokine receptor comprises a first polypeptide comprising the amino acid set for in SEQ ID NO: 122 or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 122. In certain embodiments, the nucleotide sequence that encodes the first polypeptide of the chimeric cytokine receptor comprises the nucleotide sequence that encodes the amino acid sequence of SEQ ID NO: 122, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 122. In certain embodiments, the nucleotide sequence that encodes the first polypeptide of the chimeric cytokine receptor comprises the nucleotide sequence set forth in SEQ ID NO: 123, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 123. In certain embodiments, the first polypeptide of the chimeric cytokine receptor comprises the amino acid sequence set forth in SEQ ID NO: 122. In certain embodiments, the nucleotide sequence that encodes the first polypeptide of the chimeric cytokine receptor comprises the nucleotide sequence set forth in SEQ ID NO: 123.

In some embodiments, the chimeric cytokine receptor comprises a second polypeptide comprising the amino acid set for in SEQ ID NO: 132, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 132. In certain embodiments, the nucleotide sequence that encodes the second polypeptide of the chimeric cytokine receptor comprises the nucleotide sequence that encodes the amino acid sequence of SEQ ID NO: 132, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 132. In certain embodiments, the nucleotide sequence that encodes the second polypeptide of the chimeric cytokine receptor comprises the nucleotide sequence set forth in SEQ ID NO: 133, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 133. In certain embodiments, the second polypeptide of the chimeric cytokine receptor comprises the amino acid sequence set forth in SEQ ID NO: 132. In certain embodiments, the nucleotide sequence that encodes the second polypeptide of the chimeric cytokine receptor comprises the nucleotide sequence set forth in SEQ ID NO: 133.

In certain embodiments, the first polypeptide may comprise a first extracellular region comprising one or more leucine zipper motifs comprising the amino acid sequence set forth in SEQ ID NO: 158 or variant thereof, which may be encoded by the nucleotide sequence set forth in SEQ ID NO: 159; optionally, a first hinge region of a first cytokine receptor chain comprising the amino acid sequence set forth in SEQ ID NO: 114 or variant thereof, which may be encoded by the nucleotide sequence set forth in SEQ ID NO: 115; a first transmembrane region of a first cytokine receptor chain comprising the amino acid sequence set forth in SEQ ID NO: 116 or variant thereof, which may be encoded by the nucleotide sequence set forth in SEQ ID NO: 117; a first intracellular signaling region of a first cytokine receptor chain comprising the amino acid sequence set forth in SEQ ID NO: 118 or variant thereof, which may be encoded by the nucleotide sequence set forth in SEQ ID NO: 119.

In certain embodiments, the second polypeptide may comprise a second extracellular region comprising one or more leucine zipper motifs comprising the amino acid sequence set forth in SEQ ID NO: 160 or variant thereof, which may be encoded by the nucleotide sequence set forth in SEQ ID NO: 161; optionally, a second hinge region of a second cytokine receptor chain comprising the amino acid sequence set forth in SEQ ID NO: 124 or variant thereof, which may be encoded by the nucleotide sequence set forth in SEQ ID NO: 125; a second transmembrane region of a second cytokine receptor chain comprising the amino acid sequence set forth in SEQ ID NO: 126 or variant thereof, which may be encoded by the nucleotide sequence set forth in SEQ ID NO: 127; a second intracellular signaling region of a second cytokine receptor chain comprising the amino acid sequence set forth in SEQ ID NO: 128 or variant thereof, which may be encoded by the nucleotide sequence set forth in SEQ ID NO: 129.

In some embodiments, when the chimeric cytokine receptor comprises a first polypeptide comprising the amino acid sequence set forth in SEQ ID NO: 122 or a variant thereof, which may be encoded by the nucleotide sequence set forth in SEQ ID NO: 123, and a second polypeptide comprising the amino acid sequence set forth in SEQ ID NO 132, or variant thereof, which may be encoded by the nucleotide sequence set forth in SEQ ID NO: 133, the one or more leucine zipper motifs from first extracellular region may heterodimerize with the one or more leucine zipper motifs from the second extracellular region. In certain embodiments, the chimeric cytokine receptor comprises a first polypeptide comprising the amino acid sequence set forth in SEQ ID NO: 122 or a variant thereof, and a second polypeptide comprising the amino acid sequence set forth in SEQ ID NO 132, or variant thereof. In certain embodiments, the nucleotide sequence that encodes the first polypeptide of the chimeric cytokine receptor comprises the nucleotide sequence set forth in SEQ ID NO: 123. In certain embodiments, the nucleotide sequence that encodes the second polypeptide of the chimeric cytokine receptor comprises the nucleotide sequence set forth in SEQ ID NO: 133.

In some embodiments, the chimeric cytokine receptor comprises
a) a first polypeptide comprising:
   i) a first extracellular region comprising one or more leucine zipper motifs (e.g., two leucine zipper motifs),
   ii) optionally, a first hinge region derived from the IL-27 receptor alpha chain (or WSX-1),
   iii) a first transmembrane region derived from the IL-27 receptor alpha chain (or WSX-1), and
   iv) a first intracellular signaling region of the IL-27 receptor alpha chain (or WSX-1); and
b) a second polypeptide comprising:
   i) a second extracellular region comprising one or more leucine zipper motifs (e.g., two leucine zipper motifs),
   ii) optionally, a second hinge region derived from glycoprotein 130 (gp130 or IL-6 beta chain),
   iii) a second transmembrane region derived from glycoprotein 130 (gp130 or IL-6 beta chain), and
   iv) a second intracellular signaling region derived from glycoprotein 130 (gp130 or IL-6 beta chain).

In some embodiments, the first extracellular region of the chimeric cytokine receptor comprises the amino acid sequence of SEQ ID NO: 158, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 158. In some embodiments, the nucleotide sequence that encodes the first extracellular region of the chimeric cytokine receptor comprises the nucleotide sequence that encodes the amino acid sequence of SEQ ID NO: 158, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 158. In some embodiments, the nucleotide sequence that encodes the first extracellular region of the chimeric cytokine receptor comprises the nucleotide sequence set forth in SEQ ID NO: 159, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 159. In some embodiments, the first extracellular region of the chimeric cytokine receptor comprises the amino acid sequence set forth in SEQ ID NO: 158. In some embodiments, the nucleotide sequence that encodes the first polypeptide of the chimeric cytokine receptor comprises the nucleotide sequence set forth in SEQ ID NO: 159.

In some embodiments, the first hinge region derived from the IL-27 receptor alpha chain (or WSX-1) chain comprises the amino acid sequence of SEQ ID NO: 144, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 144. In some embodiments, the nucleotide sequence that encodes the first hinge region derived from the IL-27 receptor alpha chain (or WSX-1) chain comprises the nucleotide sequence that encodes the amino acid sequence of SEQ ID NO: 144, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 144. In some embodiments, the nucleotide sequence that encodes the first hinge region derived from the IL-27 receptor alpha chain (or WSX-1) chain comprises the nucleotide sequence set forth in SEQ ID NO: 145, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 145. In some embodiments, the first hinge region derived from the TL-27 receptor alpha chain (or WSX-1) chain comprises the amino acid sequence set forth in SEQ ID NO: 144. In some embodiments, the nucleotide sequence that encodes the first hinge region derived from the TL-27 receptor alpha chain (or WSX-1) chain comprises the nucleotide sequence set forth in SEQ ID NO: 145.

In some embodiments, the first transmembrane region derived from the TL-27 receptor alpha chain (or WSX-1) chain comprises the amino acid sequence of SEQ ID NO: 146, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 146. In some embodiments, the nucleotide sequence that encodes the first transmembrane region derived from the TL-27 receptor alpha chain (or WSX-1) chain comprises the nucleotide sequence that encodes the amino acid sequence of SEQ ID NO: 146, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 146. In some embodiments, the nucleotide sequence that encodes the first transmembrane region derived from the IL-27 receptor alpha chain (or WSX-1) chain comprises the nucleotide sequence set forth in SEQ ID NO: 147, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 147. In some embodiments, the first transmembrane region derived from the IL-27 receptor alpha chain (or WSX-1) chain comprises the amino acid sequence set forth in SEQ ID NO: 146. In some embodiments, the nucleotide sequence that encodes the first transmembrane region derived from the IL-27 receptor alpha chain (or WSX-1) chain comprises the nucleotide sequence set forth in SEQ ID NO: 147.

In some embodiments, the first intracellular signaling region derived from the IL-27 receptor alpha chain (or WSX-1) chain comprises the amino acid sequence of SEQ ID NO: 148, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 148. In some embodiments, the nucleotide sequence that encodes the first intracellular signaling region derived from the IL-27 receptor alpha chain (or WSX-1) chain comprises the nucleotide sequence that encodes the amino acid sequence of SEQ ID NO: 148, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 148. In some embodiments, the nucleotide sequence that encodes the first intracellular signaling region derived from the IL-27 receptor alpha chain (or WSX-1) chain comprises the nucleotide sequence set forth in SEQ ID NO: 149, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 149. In some embodiments, the first intracellular signaling region derived from the IL-27 receptor alpha chain (or WSX-1) chain comprises the amino acid sequence set forth in SEQ ID NO: 148. In some embodiments, the nucleotide sequence that encodes the first intracellular signaling region derived from the IL-27 receptor alpha chain (or WSX-1) chain comprises the nucleotide sequence set forth in SEQ ID NO: 149.

In certain embodiment, the second extracellular region of the chimeric cytokine receptor comprises the amino acid sequence of SEQ ID NO: 160, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 160. In certain embodiments, the nucleotide sequence that encodes the second extracellular region of the chimeric cytokine receptor comprises the nucleotide sequence that encodes the amino acid sequence of SEQ ID NO: 160, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 160. In certain embodiments, the nucleotide sequence that encodes the second extracellular region of the chimeric cytokine receptor comprises the nucleotide sequence set forth in SEQ ID NO: 161, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 161. In certain embodiments, the second extracellular region of the chimeric cytokine receptor comprises the amino acid sequence set forth in SEQ ID NO: 160. In certain embodiments, the nucleotide sequence that encodes the second polypeptide of the chimeric cytokine receptor comprises the nucleotide sequence set forth in SEQ ID NO: 161.

In some embodiments, the second hinge region derived from the glycoprotein 130 (gp130 or IL-6 beta) chain comprises the amino acid sequence of SEQ ID NO: 134, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 134. In certain embodiments, the nucleotide sequence that encodes the second hinge region derived from the glycoprotein 130 (gp130 or IL-6 beta) chain comprises the nucleotide sequence that encodes the amino acid sequence of SEQ ID NO: 134, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 134. In certain embodiments, the nucleotide sequence that encodes the second hinge region derived from the glycoprotein 130 (gp130 or IL-6 beta) chain comprises the nucleotide sequence set forth in SEQ ID NO: 135, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 135. In certain embodiments, the second hinge region derived from the glycoprotein 130 (gp130 or IL-6 beta) chain comprises the amino acid sequence set forth in SEQ ID NO: 134. In certain embodiments, the nucleotide sequence that encodes the second hinge region derived from the glycoprotein 130 (gp130 or IL-6 beta) chain comprises the nucleotide sequence set forth in SEQ ID NO: 135.

In some embodiments, the second transmembrane region derived from the glycoprotein 130 (gp130 or IL-6 beta) chain comprises the amino acid sequence of SEQ ID NO: 136, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 136. In certain embodiments, the nucleotide sequence that encodes the second transmembrane region derived from the glycoprotein 130 (gp130 or IL-6 beta) chain comprises the nucleotide sequence that encodes the amino acid sequence of SEQ ID NO: 136, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 136. In certain embodiments, the nucleotide sequence that encodes the second transmembrane region derived from the glycoprotein 130 (gp130 or IL-6 beta) chain comprises the nucleotide sequence set forth in SEQ ID NO: 137, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 137. In certain embodiments, the second transmembrane region derived from the glycoprotein 130 (gp130 or IL-6 beta) chain comprises the amino acid sequence set forth in SEQ ID NO: 137. In certain embodiments, the nucleotide sequence that encodes the second transmembrane region derived from the glycoprotein 130 (gp130 or IL-6 beta) chain comprises s the nucleotide sequence set forth in SEQ ID NO: 136.

In some embodiments, the second intracellular signaling region derived from the glycoprotein 130 (gp130 or IL-6 beta) chain comprises the amino acid sequence of SEQ ID NO: 138, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 138. In certain embodiments, the nucleotide sequence that encodes the second intracellular signaling region derived from the glycoprotein 130 (gp130 or IL-6 beta) chain comprises the nucleotide sequence that encodes the amino acid sequence of SEQ ID NO: 138, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 138. In certain embodiments, the nucleotide sequence that encodes the second intracellular signaling region derived from the glycoprotein 130 (gp130 or IL-6 beta) chain comprises the nucleotide sequence set forth in SEQ ID NO: 139, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 139. In certain embodiments, the second intracellular signaling region derived from the glycoprotein 130 (gp130 or IL-6 beta) chain comprises the amino acid sequence set forth in SEQ ID NO: 138. In certain embodiments, the nucleotide sequence that encodes the second intracellular signaling region derived from the glycoprotein 130 (gp130 or IL-6 beta) chain comprises the nucleotide sequence set forth in SEQ ID NO: 139.

In some embodiments, the chimeric cytokine receptor comprises a first polypeptide comprising the amino acid set for in SEQ ID NO: 152, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 152. In certain embodiments, the nucleotide sequence that encodes the first polypeptide of the chimeric cytokine receptor comprises the nucleotide sequence that encodes the amino acid sequence of SEQ ID NO: 152, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 152. In certain embodiments, the nucleotide sequence that encodes the first polypeptide of the chimeric cytokine receptor comprises the nucleotide sequence set forth in SEQ ID NO: 153, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 153. In certain embodiments, the first polypeptide of the chimeric cytokine receptor comprises the amino acid sequence set forth in SEQ ID NO: 152. In certain embodiments, the nucleotide sequence that encodes the first polypeptide of the chimeric cytokine receptor comprises the nucleotide sequence set forth in SEQ ID NO: 153.

In some embodiments, the chimeric cytokine receptor comprises a second polypeptide comprising the amino acid set for in SEQ ID NO: 142, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 142. In certain embodiments, the nucleotide sequence that encodes the second polypeptide of the chimeric cytokine receptor comprises the nucleotide sequence that encodes the amino acid sequence of SEQ ID NO: 142, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 142. In certain embodiments, the nucleotide sequence that encodes the second polypeptide of the chimeric cytokine receptor comprises the nucleotide sequence set forth in SEQ ID NO: 143, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 143. In certain embodiments, the second polypeptide of the chimeric cytokine receptor comprises the amino acid sequence set forth in SEQ ID NO: 142. In certain embodiments, the nucleotide sequence that encodes the second polypeptide of the chimeric cytokine receptor comprises the nucleotide sequence set forth in SEQ ID NO: 143.

In certain embodiments, the first polypeptide may comprise a first extracellular region comprising one or more leucine zipper motifs comprising the amino acid sequence set forth in SEQ ID NO: 158 or variant thereof, which may be encoded by the nucleotide sequence set forth in SEQ ID NO: 159; optionally, a first hinge region of a first cytokine receptor chain comprising the amino acid sequence set forth in SEQ ID NO: 134 or variant thereof, which may be encoded by the nucleotide sequence set forth in SEQ ID NO: 135; a first transmembrane region of a first cytokine receptor chain comprising the amino acid sequence set forth in SEQ ID NO: 136 or variant thereof, which may be encoded by the nucleotide sequence set forth in SEQ ID NO: 137; a first intracellular signaling region of a first cytokine receptor chain comprising the amino acid sequence set forth in SEQ ID NO: 138 or variant thereof, which may be encoded by the nucleotide sequence set forth in SEQ ID NO: 139.

In certain embodiments, the second polypeptide may comprise a second extracellular region comprising one or more leucine zipper motifs comprising the amino acid sequence set forth in SEQ ID NO: 160 or variant thereof, which may be encoded by the nucleotide sequence set forth in SEQ ID NO: 161; optionally, a second hinge region of a second cytokine receptor chain comprising the amino acid sequence set forth in SEQ ID NO: 134 or variant thereof, which may be encoded by the nucleotide sequence set forth in SEQ ID NO: 135; a second transmembrane region of a second cytokine receptor chain comprising the amino acid sequence set forth in SEQ ID NO: 136 or variant thereof, which may be encoded by the nucleotide sequence set forth in SEQ ID NO: 137; a second intracellular signaling region of a second cytokine receptor chain comprising the amino acid sequence set forth in SEQ ID NO: 138 or variant thereof, which may be encoded by the nucleotide sequence set forth in SEQ ID NO: 139.

In some embodiments, when the chimeric cytokine receptor comprises a first polypeptide comprising the amino acid sequence set forth in SEQ ID NO: 142 or a variant thereof, which may be encoded by the nucleotide sequence set forth in SEQ ID NO: 143, and a second polypeptide comprising the amino acid sequence set forth in SEQ ID NO 132, or variant thereof, which may be encoded by the nucleotide sequence set forth in SEQ ID NO: 133, the one or more leucine zipper motifs from first extracellular region may heterodimerize with the one or more leucine zipper motifs from the second extracellular region. In certain embodiments, the chimeric cytokine receptor comprises a first polypeptide comprising the amino acid sequence set forth in SEQ ID NO: 142 or a variant thereof, and a second polypeptide comprising the amino acid sequence set forth in SEQ ID NO 132, or variant thereof. In certain embodiments, the nucleotide sequence that encodes the first polypeptide of the chimeric cytokine receptor comprises the nucleotide sequence set forth in SEQ ID NO: 143. In certain embodiments, the nucleotide sequence that encodes the second polypeptide of the chimeric cytokine receptor comprises the nucleotide sequence set forth in SEQ ID NO: 133.

In some embodiments, the chimeric cytokine receptor comprises
a) a first polypeptide comprising:
   i) a first extracellular region comprising one or more leucine zipper motifs (e.g., two leucine zipper motifs),
   ii) optionally, a first hinge region derived from the common gamma chain (γc),
   iii) a first transmembrane region derived from the common gamma chain (γc), and
   iv) a first intracellular signaling region of the common gamma chain (γc); and
b) a second polypeptide comprising:
   i) a second extracellular region comprising one or more leucine zipper motifs (e.g., two leucine zipper motifs),
   ii) optionally, a second hinge region derived from the IL-2 receptor beta chain,
   iii) a second transmembrane region derived from the IL-2 receptor beta, and
   iv) a second intracellular signaling region derived from the IL-2 receptor beta chain. v.) a third intracellular signaling region derived from the IL-21 receptor chain

In some embodiments, the first extracellular region of the chimeric cytokine receptor comprises the amino acid sequence of SEQ ID NO: 158, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 158. In some embodiments, the nucleotide sequence that encodes the first extracellular region of the chimeric cytokine receptor comprises the nucleotide sequence that encodes the amino acid sequence of SEQ ID NO: 158, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 158. In some embodiments, the nucleotide sequence that encodes the first extracellular region of the chimeric cytokine receptor comprises the nucleotide sequence set forth in SEQ ID NO: 159, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 159. In some embodiments, the first extracellular region of the chimeric cytokine receptor comprises the amino acid sequence set forth in SEQ ID NO: 158. In some embodiments, the nucleotide sequence that encodes the first polypeptide of the chimeric cytokine receptor comprises the nucleotide sequence set forth in SEQ ID NO: 159.

In some embodiments, the first hinge region derived from the common gamma chain (7c) comprises the amino acid sequence of SEQ ID NO: 30, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 30. In some embodiments, the nucleotide sequence that encodes the first hinge region derived from the common gamma chain (7c) comprises the nucleotide sequence that encodes the amino acid sequence of SEQ ID NO: 30, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 30. In some embodiments, the nucleotide sequence that encodes the first hinge region derived from the common gamma chain (γc) comprises the nucleotide sequence set forth in SEQ ID NO: 31, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 31. In some embodiments, the first hinge region derived from the common gamma chain (γc) comprises the amino acid sequence set forth in SEQ ID NO: 30. In some embodiments, the nucleotide sequence that encodes the first hinge region derived from the common gamma chain (γc) comprises the nucleotide sequence set forth in SEQ ID NO: 31.

In some embodiments, the first transmembrane region derived from the common gamma chain (γc) comprises the amino acid sequence of SEQ ID NO: 32, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 32. In some embodiments, the nucleotide sequence that encodes the first transmembrane region derived from the common gamma chain (γc) comprises the nucleotide sequence that encodes the amino acid sequence of SEQ ID NO: 32, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 32. In some embodiments, the nucleotide sequence that encodes the first transmembrane region derived from the common gamma chain (γc) comprises the nucleotide sequence set forth in SEQ ID NO: 33, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 33. In some embodiments, the first transmembrane region derived from the common gamma chain (γc) comprises the amino acid sequence set forth in SEQ ID NO: 32. In some embodiments, the nucleotide sequence that encodes the first transmembrane region derived from the common gamma chain (γc) comprises the nucleotide sequence set forth in SEQ ID NO: 33.

In some embodiments, the first intracellular signaling region derived from the common gamma chain (γc) comprises the amino acid sequence of SEQ ID NO: 34, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 34. In some embodiments, the nucleotide sequence that encodes the first intracellular signaling region derived from the common gamma chain (γc) comprises the nucleotide sequence that encodes the amino acid sequence of SEQ ID NO: 34, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 34. In some embodiments, the nucleotide sequence that encodes the first intracellular signaling region derived from the common gamma chain (γc) comprises the nucleotide sequence set forth in SEQ ID NO: 35, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 35. In some embodiments, the first intracellular signaling region derived from the common gamma chain (γc) comprises the amino acid sequence set forth in SEQ ID NO: 34. In some embodiments, the nucleotide sequence that encodes the first intracellular signaling region derived from the common gamma chain (γc) comprises the nucleotide sequence set forth in SEQ ID NO: 35

In certain embodiment, the second extracellular region of the chimeric cytokine receptor comprises the amino acid sequence of SEQ ID NO: 160, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 160. In certain embodiments, the nucleotide sequence that encodes the second extracellular region of the chimeric cytokine receptor comprises the nucleotide sequence that encodes the amino acid sequence of SEQ ID NO: 160, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 160. In certain embodiments, the nucleotide sequence that encodes the second extracellular region of the chimeric cytokine receptor comprises the nucleotide sequence set forth in SEQ ID NO: 161, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 161. In certain embodiments, the second extracellular region of the chimeric cytokine receptor comprises the amino acid sequence set forth in SEQ ID NO: 160. In certain embodiments, the nucleotide sequence that encodes the second polypeptide of the chimeric cytokine receptor comprises the nucleotide sequence set forth in SEQ ID NO: 161.

In some embodiments, the second hinge region derived from the IL-2 receptor beta chain comprises the amino acid sequence of SEQ ID NO: 14, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 14. In certain embodiments, the nucleotide sequence that encodes the second hinge region derived from the IL-2 receptor beta chain comprises the nucleotide sequence that encodes the amino acid sequence of SEQ ID NO: 14, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 14. In certain embodiments, the nucleotide sequence that encodes the second hinge region derived from the IL-2 receptor beta chain comprises the nucleotide sequence set forth in SEQ ID NO: 15, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 15. In certain embodiments, the second hinge region derived from the IL-2 receptor beta chain comprises the amino acid sequence set forth in SEQ ID NO: 14. In certain embodiments, the nucleotide sequence that encodes the second hinge region derived from the IL-2 receptor beta chain comprises the nucleotide sequence set forth in SEQ ID NO: 15.

In some embodiments, the second transmembrane region derived from the IL-2 receptor beta chain comprises the amino acid sequence of SEQ ID NO: 16, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 16. In certain embodiments, the nucleotide sequence that encodes the second transmembrane region derived from the IL-2 receptor beta chain comprises the nucleotide sequence that encodes the amino acid sequence of SEQ ID NO: 16, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 16. In certain embodiments, the nucleotide sequence that encodes the second transmembrane region derived from the IL-2 receptor beta chain comprises the nucleotide sequence set forth in SEQ ID NO: 17, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 17. In certain embodiments, the second transmembrane region derived from the IL-2 receptor beta chain comprises the amino acid sequence set forth in SEQ ID NO: 16. In certain embodiments, the nucleotide sequence that encodes the second transmembrane region derived from the TL-2 receptor beta chain comprises s the nucleotide sequence set forth in SEQ ID NO: 17.

In some embodiments, the second intracellular signaling region derived from the IL-2 receptor beta chain comprises the amino acid sequence of SEQ ID NO: 18, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 18. In certain embodiments, the nucleotide sequence that encodes the second intracellular signaling region derived from the IL-2 receptor beta chain comprises the nucleotide sequence that encodes the amino acid sequence of SEQ ID NO: 18, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 18. In certain embodiments, the nucleotide sequence that encodes the second intracellular signaling region derived from the IL-2 receptor beta chain comprises the nucleotide sequence set forth in SEQ ID NO: 19, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 19. In certain embodiments, the second intracellular signaling region derived from the IL-2 receptor beta chain comprises the amino acid sequence set forth in SEQ ID NO: 18. In certain embodiments, the nucleotide sequence that encodes the second intracellular signaling region derived from the IL-2 receptor beta chain comprises the nucleotide sequence set forth in SEQ ID NO: 19.

In some embodiments, the third intracellular signaling region derived from the IL-21 receptor chain comprises the amino acid sequence of SEQ ID NO: 60, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 60. In certain embodiments, the nucleotide sequence that encodes the third intracellular signaling region derived from the IL-21 receptor chain comprises the nucleotide sequence that encodes the amino acid sequence of SEQ ID NO: 60, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 60. In certain embodiments, the nucleotide sequence that encodes the third intracellular signaling region derived from the IL-21 receptor chain comprises the nucleotide sequence set forth in SEQ ID NO: 61, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 61. In certain embodiments, the third intracellular signaling region derived from the IL-21 receptor chain comprises the amino acid sequence set forth in SEQ ID NO: 60. In certain embodiments, the nucleotide sequence that encodes the third intracellular signaling region derived from the IL-21 receptor chain comprises the nucleotide sequence set forth in SEQ ID NO: 61.

In some embodiments, the chimeric cytokine receptor comprises a first polypeptide comprising the amino acid set for in SEQ ID NO: 156, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 156. In certain embodiments, the nucleotide sequence that encodes the first polypeptide of the chimeric cytokine receptor comprises the nucleotide sequence that encodes the amino acid sequence of SEQ ID NO: 156, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 156. In certain embodiments, the nucleotide sequence that encodes the first polypeptide of the chimeric cytokine receptor comprises the nucleotide sequence set forth in SEQ ID NO: 157, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 157. In certain embodiments, the first polypeptide of the chimeric cytokine receptor comprises the amino acid sequence set forth in SEQ ID NO: 156. In certain embodiments, the nucleotide sequence that encodes the first polypeptide of the chimeric cytokine receptor comprises the nucleotide sequence set forth in SEQ ID NO: 157.

In some embodiments, the chimeric cytokine receptor comprises a second polypeptide comprising the amino acid set for in SEQ ID NO: 154, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 154. In certain embodiments, the nucleotide sequence that encodes the second polypeptide of the chimeric cytokine receptor comprises the nucleotide sequence that encodes the amino acid sequence of SEQ ID NO: 154, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 154. In certain embodiments, the nucleotide sequence that encodes the second polypeptide of the chimeric cytokine receptor comprises the nucleotide sequence set forth in SEQ ID NO: 155, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 155. In certain embodiments, the second polypeptide of the chimeric cytokine receptor comprises the amino acid sequence set forth in SEQ ID NO: 154. In certain embodiments, the nucleotide sequence that encodes the second polypeptide of the chimeric cytokine receptor comprises the nucleotide sequence set forth in SEQ ID NO: 155.

In certain embodiments, the first polypeptide may comprise a first extracellular region comprising one or more leucine zipper motifs comprising the amino acid sequence set forth in SEQ ID NO: 158 or variant thereof, which may be encoded by the nucleotide sequence set forth in SEQ ID NO: 159; optionally, a first hinge region of a first cytokine receptor chain comprising the amino acid sequence set forth in SEQ ID NO: 30 or variant thereof, which may be encoded by the nucleotide sequence set forth in SEQ ID NO: 31; a first transmembrane region of a first cytokine receptor chain comprising the amino acid sequence set forth in SEQ ID NO: 32 or variant thereof, which may be encoded by the nucleotide sequence set forth in SEQ ID NO: 33; a first intracellular signaling region of a first cytokine receptor chain comprising the amino acid sequence set forth in SEQ ID NO: 34 or variant thereof, which may be encoded by the nucleotide sequence set forth in SEQ ID NO: 35.

In certain embodiments, the second polypeptide may comprise a second extracellular region comprising one or more leucine zipper motifs comprising the amino acid sequence set forth in SEQ ID NO: 160 or variant thereof, which may be encoded by the nucleotide sequence set forth in SEQ ID NO: 161; optionally, a second hinge region of a second cytokine receptor chain comprising the amino acid sequence set forth in SEQ ID NO: 14 or variant thereof, which may be encoded by the nucleotide sequence set forth in SEQ ID NO: 15; a second transmembrane region of a second cytokine receptor chain comprising the amino acid sequence set forth in SEQ ID NO: 16 or variant thereof, which may be encoded by the nucleotide sequence set forth in SEQ ID NO: 17; a second intracellular signaling region of a second cytokine receptor chain comprising the amino acid sequence set forth in SEQ ID NO: 18 or variant thereof, which may be encoded by the nucleotide sequence set forth in SEQ ID NO: 19; a third intracellular signaling region of a second cytokine receptor chain comprising the amino acid sequence set forth in SEQ ID NO: 60 or variant thereof, which may be encoded by the nucleotide sequence set forth in SEQ ID NO: 61.

In some embodiments, when the chimeric cytokine receptor comprises a first polypeptide comprising the amino acid sequence set forth in SEQ ID NO: 156 or a variant thereof, which may be encoded by the nucleotide sequence set forth in SEQ ID NO: 157, and a second polypeptide comprising the amino acid sequence set forth in SEQ ID NO 154, or variant thereof, which may be encoded by the nucleotide sequence set forth in SEQ ID NO: 155, the one or more leucine zipper motifs from first extracellular region may heterodimerize with the one or more leucine zipper motifs from the second extracellular region. In certain embodiments, the chimeric cytokine receptor comprises a first polypeptide comprising the amino acid sequence set forth in SEQ ID NO: 156 or a variant thereof, and a second polypeptide comprising the amino acid sequence set forth in SEQ ID NO 154, or variant thereof. In certain embodiments, the nucleotide sequence that encodes the first polypeptide of the chimeric cytokine receptor comprises the nucleotide sequence set forth in SEQ ID NO: 157. In certain embodiments, the nucleotide sequence that encodes the second polypeptide of the chimeric cytokine receptor comprises the nucleotide sequence set forth in SEQ ID NO: 155.

### Polynucleotides

In one aspect, the present invention provides polynucleotides encoding any of the chimeric cytokine receptor disclosed herein.

In some embodiments, the polynucleotide may comprise
a) a nucleotide sequence encoding the first polypeptide of the chimeric cytokine receptor; and
b) a nucleotide sequence encoding the second polypeptide of the chimeric cytokine receptor.

In some embodiments, the nucleotide sequence encoding the first polypeptide of the chimeric cytokine receptor may be operably linked to the first polypeptide of the chimeric cytokine receptor via a sequence encoding a self-cleaving peptide and/or an internal ribosomal entry site (IRES).

In some embodiments, the self-cleaving peptide may be a 2A peptide. Non-limiting examples of self-cleaving peptide sequences includes Thoseaasigna virus 2A (T2A; AEGRGSLLTCGDVEENPGP (SEQ ID NO: 229), EGRGSLLTCGDVEENPGP (SEQ ID NO: 230), or GSGEGRGSLLTCGDVEENPGP (SEQ ID NO: 231)); the foot and mouth disease virus (FMDV) 2A sequence (F2A; GSGSRVTELLYRMKRAETYCPRPLLAIIIPTEARHKQKIVAPVKQLLNFDLLKLAGDVES NPGP (SEQ ID NO: 232)), Sponge (Amphimedon queenslandica) 2A sequence (LLCFLLLLLSGDVELNPGP (SEQ ID NO: 233); or HHFMFLLLLLAGDIELNPGP (SEQ ID NO: 234)); acorn worm 2A sequence (Saccoglossus kowalevskii) (WFLVLLSFILSGDIEVNPGP (SEQ ID NO: 235)); amphioxus (Branchiostoma floridae) 2A sequence (KNCAMYMLLLSGDVETNPGP (SEQ ID NO: 236); or MVISQLMLKLAGDVEENPGP (SEQ ID NO: 237)); porcine teschovirus-1 2A sequence (P2A; GSGATNFSLLKQAGDVEENPGP (SEQ ID NO: 22)); and equine rhinitis A virus 2A sequence (E2A; GSGQCTNYALLKLAGDVESNPGP (SEQ ID NO: 238)). In some embodiments, the separation sequence is a naturally occurring or synthetic sequence. In certain embodiments, the separation sequence includes the 2A consensus sequence D-X-E-X-NPGP (SEQ ID NO: 239), in which X is any amino acid residue.

In some embodiments, the 2A peptide may be a P2A peptide.

In some embodiments, the P2A peptide comprises the amino acid sequence of SEQ ID NO: 22, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 22. In some embodiments, the nucleotide sequence that encodes the P2A peptide comprises the nucleotide sequence that encodes the amino acid sequence of SEQ ID NO: 22, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 22. In some embodiments, the nucleotide sequence that encodes the P2A peptide comprises the nucleotide sequence set forth in SEQ ID NO: 23, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 23. In some embodiments, the nucleotide sequence that encodes the P2A peptide comprises the nucleotide sequence set forth in SEQ ID NO: 24, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 24. In some embodiments, the P2A peptide comprises the amino acid sequence set forth in SEQ ID NO: 22. In some embodiments, the nucleotide sequence that encodes P2A peptide comprises the nucleotide sequence set forth in SEQ ID NO: 23. In some embodiments, the nucleotide sequence that encodes P2A peptide comprises the nucleotide sequence set forth in SEQ ID NO: 24.

In some embodiments, the P2A peptide comprises the amino acid sequence GSGATNFSLLKQAGDVEENPGP (SEQ ID NO: 22), or an amino acid sequence having at least 80% sequence identity thereof.

In one aspect, the present disclosure provides a polynucleotide comprising a nucleotide sequence encoding the first polypeptide any of the chimeric cytokine receptors disclosed herein.

In one aspect, the present disclosure provides a polynucleotide comprising a nucleotide sequence encoding the second polypeptide of any of the chimeric cytokine receptors disclosed herein.

In some embodiments, the nucleotide sequence(s) may be expressed in an inducible fashion, for example, as may be achieved with an inducible promoter, an inducible expression system, an artificial signaling circuits, and/or drug-induced splicing.

In some embodiments, the nucleotide sequence(s) may be expressed in an inducible fashion, such as that which may be achieved with i) an inducible promoter, for example, but not limited to promotors that may be activated by T cell activation (e.g. NFAT, Nur66, IFNg) or hypoxia; ii) an inducible expression system, for example, but not limited to doxycycline- or tamoxifen- inducible expression system; iii) artificial signaling circuits including, but not limited to, SynNotch, and/or iv) drug-induced splicing. By way of a non-limiting example, drug-induced splicing methods and/or compositions useful in the practice of the present disclosure may be based those described in, for example, Monteys et al., 2021 [44], the contents of which is incorporated herein by reference in its entirety for all purposes.

In some embodiments, the polynucleotides encoding the chimeric cytokine receptors disclosed herein may be expressed as a “split molecule” in which for example, the leucine zipper regions, transmembrane and intracellular signaling regions of the chimeric cytokine receptors may be assembled only in the presence of a heterodimerizing small molecule, as described in, for example, Wu et al., 2015 [45], the contents of which is incorporated herein by reference in its entirety for all purposes.

In some embodiments, the nucleotide sequence (s) may encode a moiety so that the stability of the chimeric cytokine receptor may be regulated with a small molecule, including but not limited to, the “SWIFF” technology or an immunomodulatory drug (IMiD)-inducible degron as described, for example, in Juillerat et al., 2019 [46], Carbonneau et al., 2021 [47], and Jan et al., 2021 [48], the contents of each of which is incorporated herein by reference in its entirety for all purposes.

In some embodiments, the nucleotide sequences encoding the first and/or second polypeptides of the chimeric cytokine receptor may be operably linked to at least a regulatory element. The regulatory element may be capable of mediating expression of the first and/or second polypeptides of the chimeric cytokine receptor. Regulatory elements include, but are not limited to, promoters, enhancers, initiation sites, polyadenylation (polyA) tails, IRES elements, response elements, and termination signals. In certain embodiments, the regulatory element regulates chimeric cytokine receptor expression. In certain embodiments, the regulatory element increased the expression of the chimeric cytokine receptor construct. In certain embodiments, the regulatory element increased the expression of the chimeric cytokine receptor construct once the host cell is activated. In certain embodiments, the regulatory element decreases expression of the chimeric cytokine receptor construct. In certain embodiments, the regulatory element decreases expression of the chimeric cytokine receptor construct once the host cell is activated

In some embodiments, the nucleotide sequences encoding the first and/or second polypeptides of the chimeric cytokine receptor may be operably linked to a single promoter.

In some embodiments, the nucleotide sequence encoding the first polypeptide of the chimeric cytokine receptor may be operably linked to a first promoter. In some embodiments, the nucleotide sequence encoding the second polypeptide of the chimeric cytokine receptor may be operably linked to a second promoter.

In some embodiments, the nucleotide sequence encoding the first polypeptide of the chimeric cytokine receptor may be operably linked to a first promoter, the nucleotide sequence encoding the second polypeptide of the chimeric cytokine receptor may be operably linked to a second promoter, and the first and second promoters are the same.

In some embodiments, the nucleotide sequence encoding the first polypeptide of the chimeric cytokine receptor may be operably linked to a first promoter, the nucleotide sequence encoding the second polypeptide of the chimeric cytokine receptor may be operably linked to a second promoter, and the first and second promoters are different.

In some embodiments, the promoter is an inducible promoter. Non-limiting examples of an inducible promoter are lac, sp6, T7, and Hsp70- and Hsp90- derived promoters.

In some embodiments, the inducible promoter is a tetracycline (Tc)-inducible promoter.

In some embodiments, the promoter may be a T cell-specific promoter or an NK cell-specific promoter.

### Additional Genes

In addition to the chimeric cytokine receptor construct, the polynucleotide may further comprise at least one additional gene that encodes an additional peptide. Examples of additional genes can include a transduced host cell selection marker, an in vivo tracking marker, a cytokine, a suicide gene, or some other functional gene. In certain embodiments, the functional additional gene can induce the expression of another molecule. In certain embodiments, the functional additional gene can increase the safety of the chimeric cytokine receptor. For example, the chimeric cytokine receptor construct may comprise an additional gene which is truncated CD19 (tCD19). The tCD19 can be used as a tag. Expression of tCD19 may also help determine transduction efficiency. In some embodiments, the functional additional gene may allow for regulation of the stability of the chimeric cytokine receptor.

Non-limiting examples of classes of additional genes that can be used to increase the effector function of the modified host cells, include (a) secretable cytokines (e.g., but not limited to, GM-CSF, IL-7, IL-12, IL-15, IL-18), (b) membrane bound cytokines (e.g., but not limited to, IL-15), (c) other chimeric cytokine receptors (e.g., but not limited to, IL-2/IL-7, IL-4/IL-7), (d) constitutive active cytokine receptors (e.g., but not limited to, C7R), (e) dominant negative receptors (DNR; e.g., but not limited to TGFRII DNR), (f) ligands of costimulatory molecules (e.g., but not limited to, CD80, 4-1BBL), (g) nuclear factor of activated T-cells (NFATs) (e.g., NFATc1, NFATc2, NFATc3, NFATc4, and NFAT5), (h) antibodies, including fragments thereof and bispecific antibodies (e.g., but not limited to, bispecific T-cell engagers (BiTEs)), (i) chimeric antigen receptors (CARs), or (j) safety switches or suicide genes (e.g., CD20, truncated EGFR or HER2, inducible caspase 9 molecules).

In certain embodiments, the chimeric cytokine receptor construct may comprise an additional gene that encodes GM-CSF. The expression of exogenous GM-CSF may further enhance the function of the host cells expressing the chimeric cytokine receptor of the present disclosure.

In certain embodiments, the chimeric cytokine receptor construct may comprise at least one additional gene that encodes, for example, without limitation one or more cellular markers, epitope tags, cytokines, safety switches, dimerization moieties, or degradation moieties.

In some embodiments, the additional gene is an epitope tag. As a non-limiting example, the epitope tag is FLAG. As another non-limiting example, the epitope tag is Myc.

In some embodiments, the additional gene is a cellular marker. As a non-limiting example, the cellular marker is mClover3. As another non-limiting example, the cellular marker is mRuby.

In some embodiments, the additional gene may encode a moiety so that the stability of the chimeric cytokine receptor may be regulated with a small molecule, including but not limited to, the “SWIFF” technology or an immunomodulatory drug (IMiD)-inducible degron as described, for example, in Juillerat et al., 2019 [46], Carbonneau et al., 2021 [47], and Jan et al., 2021 [48], the contents of each of which is incorporated herein by reference in its entirety for all purposes.

In certain embodiments, the functional additional gene is a suicide gene. A suicide gene is a recombinant gene that will cause the host cell that the gene is expressed in to undergo programmed cell death or antibody mediated clearance at a desired time. Suicide genes can function to increase the safety of the chimeric cytokine receptor. In another embodiment, the additional gene is an inducible suicide gene. Non-limiting examples of suicide genes include i) molecules that are expressed on the cell surface and can be targeted with a clinical grade monoclonal antibody including CD20, EGFR or a fragment thereof, HER2 or a fragment thereof, and ii) inducible suicide genes (e.g., but not limited to inducible caspase 9 (see Straathof et al. (2005) Blood. 105(11): 4247-4254; US Publ. No. 2011/0286980, each of which are incorporated herein by reference in their entirety for all purposes)).

In certain aspects, chimeric cytokine receptors of the present disclosure may be regulated by a safety switch. As used herein, the term “safety switch” refers to any mechanism that is capable of removing or inhibiting the effect of a chimeric cytokine receptor from a system (e.g., a culture or a subject). Safety switches can function to increase the safety of the chimeric cytokine receptor.

The function of the safety switch may be inducible. Non-limiting examples of safety switches include (a) molecules that are expressed on the cell surface and can be targeted with a clinical grade monoclonal antibody including CD20, EGFR or a fragment thereof, HER2 or a fragment thereof, and (b) inducible suicide genes (e.g., but not limited to herpes simplex virus thymidine kinase (HSV-TK) and inducible caspase 9 (see Straathof et al. (2005) Blood. 105(11): 4247-4254; US Publ. No. 2011/0286980, each of which are incorporated herein by reference in their entirety for all purposes).

In some embodiments, the safety switch is a CD20 polypeptide. Expression of human CD20 on the cell surface presents an attractive strategy for a safety switch. The inventors and others have shown that cells that express CD20 can be rapidly eliminated with the FDA approved monoclonal antibody rituximab through complement-mediated cytotoxicity and antibody-dependent cell-mediated cytotoxicity (see e.g., Griffioen, M., et al. Haematologica 94, 1316-1320 (2009), which is incorporated herein by reference in its entirety for all purposes). Rituximab is an anti-CD20 monoclonal antibody that has been FDA approved for Chronic Lymphocytic Leukemia (CLL) and Non-Hodgkin's Lymphoma (NHL), among others (Storz, U. MAbs 6, 820-837 (2014), which is incorporated herein by reference in its entirety for all purposes). The CD20 safety switch is non-immunogenic and can function as a reporter/selection marker in addition to a safety switch (Bonifant, C. L., et al. Mol Ther 24, 1615-1626 (2016); van Loenen, M. M., et al. Gene Ther 20, 861-867 (2013); each of which is incorporated herein by reference in its entirety for all purposes).

In some embodiments, the chimeric cytokine receptor may be expressed in an inducible fashion, such as that which may be achieved with i) an inducible promoter, for example, but not limited to promotors that may be activated by T cell activation (e.g. NFAT, Nur66, IFNy) or hypoxia; ii) an inducible expression system, for example, but not limited to doxycycline- or tamoxifen- inducible expression system; iii) artificial signaling circuits including, but not limited to, SynNotch, and/or iv) drug-induced splicing. By way of a non-limiting example, drug-induced splicing methods and/or compositions useful in the practice of the present disclosure may be based those described in, for example, Monteys et al., 2021 [44], the contents of which is incorporated herein by reference in its entirety for all purposes.

In some embodiments, the first and second polypeptides of the chimeric cytokine receptor may each comprise a dimerization moiety, which may promote dimerization of the two polypeptides of the chimeric cytokine receptor in the presence of an inducer. For example, the chimeric cytokine receptor may be expressed as a ‘split molecule’ in which for example, the leucine zipper regions, transmembrane and intracellular signaling regions are only assembled in the presence of a heterodimerizing small molecule, as described in, for example, Wu et al., 2015 [45], the contents of which is incorporated herein by reference in its entirety for all purposes.

In certain embodiments, the chimeric cytokine receptor comprises at least one additional gene (i.e., a second gene). In certain embodiments, the chimeric cytokine receptor comprises one second gene. In other embodiments, the chimeric cytokine receptor comprises two additional genes (i.e., a third gene). In yet another embodiment, the chimeric cytokine receptor comprises three additional genes (i.e., a fourth gene). In certain embodiments, the additional genes are separated from each other and the chimeric cytokine receptor construct. For example, they may be separated by 2A sequences and/or an internal ribosomal entry sites (IRES) as described above. In certain examples, the chimeric cytokine receptor can be at any position of the polynucleotide chain.

### Recombinant Vectors

The present disclosure provides recombinant vectors comprising a polynucleotide encoding a chimeric cytokine receptor. Such recombinant vectors may comprise polynucleotides encoding the proteins disclosed above. In certain embodiments, the polynucleotide is operatively linked to at least one regulatory element for expression of the chimeric cytokine receptor.

In certain embodiments, the vector is a viral vector. In certain embodiments, the viral vector can be, but is not limited to, a retroviral vector, a lentiviral vector, an adenoviral vector, an adeno-associated virus vector, an alphaviral vector, a herpes virus vector, a baculoviral vector, or a vaccinia virus vector.

In some embodiments, the viral vector is a retroviral vector.

In some embodiments, the vector is a non-viral vector. Non-viral vectors suitable for use in this invention include but are not limited to minicircle plasmids, transposon systems (e.g., Sleeping Beauty transposon, piggyBac transposon), or single or double stranded DNA molecules that are used as templates for homology directed repair (HDR) based gene editing.

In some embodiments, the vector comprises a nucleotide sequence of any of SEQ ID NOs: 242, 244, 246, 248, 250, 252, 254, 256, 258, and 260, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with any of SEQ ID NOs: 242, 244, 246, 248, 250, 252, 254, 256, 258, and 260.

### Isolated Host Cells

In another aspect, provided herein is an isolated host cell comprising any of the various polynucleotides described herein or the recombinant vector described herein.

In a further aspect, provided herein is an isolated host cell comprising a chimeric cytokine receptor by the polynucleotide described herein.

In certain embodiments, the host cell is an immune cell. In various embodiments, the host cell is a T-cell. T-cells may include, but are not limited to, thymocytes, naive T lymphocytes, immature T lymphocytes, mature T lymphocytes, resting T lymphocytes, or activated T lymphocytes. A T-cell can be a T helper (Th) cell, for example a T helper 1 (Th1) or a T helper 2 (Th2) cell. The T-cell can be a helper T-cell (HTL; CD4+ T-cell) CD4+ T-cell, a cytotoxic T-cell (CTL; CD8+ T-cell), a tumor infiltrating cytotoxic T-cell (TIL; CD8+ T-cell), CD4+ CD8+ T-cell, or any other subset of T-cells. Other illustrative populations of T-cells suitable for use in particular embodiments include naive T-cells memory T-cells, NKT cells, and iNKT cells.

In some embodiments, the host cell may be a T cell, a natural killer cell (NK) cell, a mesenchymal stem cell (MSC) or a macrophage. In some embodiments, the host cell is a T cell.

In some embodiments, the T-cell is selected an αβ T-cell receptor (TCR) T-cell, a γδ T-cell, a CD8+ T-cell, a CD4+ T-cell, a cytotoxic T-cell, an invariant natural killer T (iNKT) cell, a memory T-cell, a memory stem T-cell (T_{SCM}), a naïve T-cell, an effector T-cell, a T-helper cell, or a regulatory T-cell (Treg).

In various embodiments, the host cell is a natural killer (NK) cell. NK cell refers to a differentiated lymphocyte with a CD3− CD16+, CD3− CD56+, CD16+ CD56+ and/or CD57+TCR− phenotype.

In some embodiments, the host cell is a NK cell derived from peripheral, cord blood, IPCs, induced pluripotent stem (iPS) cells, and/or a cell line (e.g., NK-92 cells).

In some embodiments, the host cell may be an immune cell. In some embodiments, the immune cell may be derived from induced pluripotent stem (iPS) cells. Non-limiting examples of iPSCs that may be used in accordance with the disclosure may be any such cells as described in, e.g., Zhu et al., 2019 [49] and Iriguchi et al., 2021 [50], the contents of each of which is incorporated herein by reference in its entirety for all purposes.

In various embodiments, other host immune cells are selected, for example, but not limited to, macrophages. In various embodiments, the host immune cell is a dendritic cell, a Langerhans cell, or a B cell. In various embodiments, the host immune cell is a professional antigen presenting cell (APC). In various embodiments, the host immune cell is a non-professional antigen presenting cell (APC).

In some embodiments, the host cell may further express one or more antigen-recognition molecules. In some embodiments, the one or more antigen-recognition molecules is an αβ T cell receptors (TCRs), a synthetic T cell receptor and antigen receptor (STAR), a chimeric antigen receptor (CAR), a T cell antigen coupler (TAC), a T cell receptor fusion construct (TruC), or an antibody (e.g., bispecific antibodies), or a combination thereof.

In some embodiments, the host cell may be further genetically modified to enhance its function by a) expressing one or more additional genes (e.g., transcription factors (c-Jun)) or deleting one or more inhibitory genes (e.g., REGNASE-1, DNMT3A) with gene editing technologies (e.g., CRISPR-Cas9 or transcription activator-like effector nucleases (TALENs)). Suitable methods of genetic modification of immune cells to knock out inhibitory genes such as REGNASE-1, and DNMT3A include those described in, e.g., WO2020/219682, WO2020/210365, which are incorporated herein by reference in their entireties.

In various embodiments, the host cell has been activated and/or expanded ex vivo.

In various embodiments, the host cell is an allogeneic cell. In various embodiments, the host cell is an autologous cell.

In certain embodiments, the host cell is isolated form a subject having a cancer. In some embodiments, the host cell is isolated from a subject having a tumor. In various embodiments, the cancer is a solid tumor, a brain tumor, or a leukemia. In some embodiments, the tumor can be found within, but not limited to, breast tissue, prostate tissue, bladder tissue, oral and/or dental tissue, head and/or neck tissue, stomach tissue, liver tissue, colorectal tissue, lung tissue, brain tissue, ovary, cervix, esophagus, skin, lymph nodes, and/or bone. In some embodiments, the tumor is a cancer. In some embodiments, the cancer can be, but not limited to, osteosarcoma, rhabdomyosarcoma, Ewing sarcoma and other Ewing sarcoma family of tumors, neuroblastoma, ganglioneuroblastoma, desmoplastic small round cell tumor, malignant peripheral nerve sheath tumor, synovial sarcoma, undifferentiated sarcoma, adrenocortical carcinoma, hepatoblastoma, Wilms tumor, rhabdoid tumor, high grade glioma (glioblastoma multiforme), medulloblastoma, astrocytoma, glioma, ependymoma, atypical teratoid rhabdoid tumor, meningioma, craniopharyngioma, primitive neuroectodermal tumor, diffuse intrinsic pontine glioma and other brain tumors, acute myeloid leukemia, multiple myeloma, lung cancer, mesothelioma, breast cancer, bladder cancer, gastric cancer, prostate cancer, colorectal cancer, endometrial cancer, cervical cancer, renal cancer, esophageal cancer, ovarian cancer, pancreatic cancer, hepatocellular carcinoma and other liver cancers, head and neck cancers, leiomyosarcoma, and melanoma.

In certain embodiments, the host cell is isolated from a subject having a tumor.

In some embodiments, the host cell is derived from a blood, marrow, tissue, or a tumor sample.

In certain aspects, the present disclosure provides a method of generating an isolated host cell described herein. The method includes genetically modifying the host cell with the polynucleotide described herein or the recombinant vector described herein. In some embodiments, the method may further comprise genetically modifying the host cell to express, e.g., a chimeric antigen receptor (CAR). In some embodiments, the genetic modifying step is conducted via viral gene delivery. In some embodiments, the genetic modifying step is conducted via non-viral gene delivery. In some embodiments, the genetically modifying step is conducted ex vivo. In some embodiments, the method further comprises activation and/or expansion of the host cell ex vivo before, after and/or during said genetic modification. In some embodiments, the method further comprises activation and/or expansion of the host cell ex vivo before, after and/or during genetic modifying step.

### Isolation/Enrichment

The host cells may be autologous/autogeneic (“self”) or non-autologous (“non-self,” e.g., allogeneic, syngeneic or xenogeneic). In certain embodiments, the host cells are obtained from a mammalian subject. In other embodiments, the host cells are obtained from a primate subject. In certain embodiments, the host cells are obtained from a human subject.

Lymphocytes can be obtained from sources such as, but not limited to, peripheral blood mononuclear cells, bone marrow, lymph nodes tissue, cord blood, thymus issue, tissue from a site of infection, ascites, pleural effusion, spleen tissue, and tumors. Lymphocytes may also be generated by differentiation of stem cells. In certain embodiments, lymphocytes can be obtained from blood collected from a subject using techniques generally known to the skilled person, such as sedimentation, e.g., FICOLL™ separation.

In certain embodiments, cells from the circulating blood of a subject are obtained by apheresis. An apheresis device typically contains lymphocytes, including T-cells, monocytes, granulocytes, B cells, other nucleated white blood cells, red blood cells, and platelets. In certain embodiments, the cells collected by apheresis may be washed to remove the plasma fraction and to place the cells in an appropriate buffer or media for subsequent processing. The cells can be washed with PBS or with another suitable solution that lacks calcium, magnesium, and most, if not all other, divalent cations. A washing step may be accomplished by methods known to those in the art, such as, but not limited to, using a semiautomated flowthrough centrifuge (e.g., Cobe 2991 cell processor, or the Baxter CytoMate). After washing, the cells may be resuspended in a variety of biocompatible buffers, cell culture medias, or other saline solution with or without buffer.

In certain embodiments, host cells can be isolated from peripheral blood mononuclear cells (PBMCs) by lysing the red blood cells and depleting the monocytes. As an example, the cells can be sorted by centrifugation through a PERCOLL™ gradient. In certain embodiments, after isolation of PBMC, both cytotoxic and helper T lymphocytes can be sorted into naive, memory, and effector T-cell subpopulations either before or after activation, expansion, and/or genetic modification.

In certain embodiments, T lymphocytes can be enriched. For example, a specific subpopulation of T lymphocytes, expressing one or more markers such as, but not limited to, CD3, CD4, CD8, CD14, CD15, CD16, CD19, CD27, CD28, CD34, CD36, CD45RA, CD45RO, CD56, CD62, CD62L, CD122, CD123, CD127, CD235a, CCR7, HLA-DR or a combination thereofusing either positive or negative selection techniques. In certain embodiments, the T lymphocytes for use in the compositions of the disclosure do not express or do not substantially express one or more of the following markers: CD57, CD244, CD160, PD-1, CTLA4, TIM3, and LAG3.

In certain embodiments, NK cells can be enriched. For example, a specific subpopulation of T lymphocytes, expressing one or more markers such as, but not limited to, CD2, CD16, CD56, CD57, CD94, CD122 or a combination thereof using either positive or negative selection techniques.

### Stimulation/Activation

In order to reach sufficient therapeutic doses of host cell compositions, host cells are often subjected to one or more rounds of stimulation/activation. In certain embodiments, a method of producing host cells for administration to a subject comprises stimulating the host cells to become activated in the presence of one or more stimulatory signals or agents (e.g., compound, small molecule, e.g., small organic molecule, nucleic acid, polypeptide, or a fragment, isoform, variant, analog, or derivative thereof). In certain embodiments, a method of producing host cells for administration to a subject comprises stimulating the host cells to become activated and to proliferate in the presence of one or more stimulatory signals or agents.

Host cells (e.g., T lymphocytes and NK cells) can be activated by inducing a change in their biologic state by which the cells express activation markers, produce cytokines, proliferate and/or become cytotoxic to target cells. All these changes can be produced by primary stimulatory signals. Co-stimulatory signals amplify the magnitude of the primary signals and suppress cell death following initial stimulation resulting in a more durable activation state and thus a higher cytotoxic capacity.

T cells can be activated generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041, each of which is incorporated herein by reference in its entirety.

In certain embodiments, the T-cell based host cells can be activated by binding to an agent that activates CD3ζ.

In other embodiments, a CD2-binding agent may be used to provide a primary stimulation signal to the T-cells. For example, and not by limitation, CD2 agents include, but are not limited to, CD2 ligands and anti-CD2 antibodies, e.g., the Tl 1.3 antibody in combination with the Tl 1.1 or Tl 1.2 antibody (Meuer, S. C. et al. (1984) Cell 36:897-906) and the 9.6 antibody (which recognizes the same epitope as TI 1.1) in combination with the 9-1 antibody (Yang, S. Y. et al. (1986) J. Immunol. 137:1097-1100). Other antibodies which bind to the same epitopes as any of the above described antibodies can also be used.

In certain embodiments, the host cells are activated by administering phorbol myristate acetate (PMA) and ionomycine. In certain embodiments, the host cells are activated by administering an appropriate antigen that induces activation and then expansion. In certain embodiments, PMA, ionomycin, and/or appropriate antigen are administered with CD3 induce activation and/or expansion.

In general, the activating agents used in the present disclosure includes, but is not limited to, an antibody, a fragment thereof and a proteinaceous binding molecule with antibody-like functions. Examples of (recombinant) antibody fragments are Fab fragments, Fv fragments, single-chain Fv fragments (scFv), a divalent antibody fragment such as an (Fab)2′-fragment, diabodies, triabodies (Iliades, P., et al., FEBS Lett (1997) 409, 437-441), decabodies (Stone, E., et al., Journal of Immunological Methods (2007) 318, 88-94) and other domain antibodies (Holt, L. J., et al., Trends Biotechnol. (2003), 21, 11, 484-490). The divalent antibody fragment may be an (Fab)2′-fragment, or a divalent single-chain Fv fragment while the monovalent antibody fragment may be selected from the group consisting of a Fab fragment, a Fv fragment, and a single-chain Fv fragment (scFv).

In certain embodiments, one or more binding sites of the CD3ζ agents may be a bivalent proteinaceous artificial binding molecule such as a dimeric lipocalin mutein (i.e., duocalin). In certain embodiments the receptor binding reagent may have a single second binding site, (i.e., monovalent). Examples of monovalent agents include, but are not limited to, a monovalent antibody fragment, a proteinaceous binding molecule with antibody-like binding properties or an MHC molecule. Examples of monovalent antibody fragments include, but are not limited to a Fab fragment, a Fv fragment, and a single-chain Fv fragment (scFv), including a divalent single-chain Fv fragment.

The agent that specifically binds CD3 includes, but is not limited to, an anti-CD3-antibody, a divalent antibody fragment of an anti-CD3 antibody, a monovalent antibody fragment of an anti-CD3-antibody, and a proteinaceous CD3-binding molecule with antibody-like binding properties. A proteinaceous CD3-binding molecule with antibody-like binding properties can be an aptamer, a mutein based on a polypeptide of the lipocalin family, a glubody, a protein based on the ankyrin scaffold, a protein based on the crystalline scaffold, an adnectin, and an avimer. It also can be coupled to a bead.

In certain embodiments, the activating agent (e.g., CD3-binding agents) can be present in a concentration of about 0.1 to about 10 μg/ml. In certain embodiments, the activating agent (e.g., CD3-binding agents) can be present in a concentration of about 0.2 μg/ml to about 9 μg/ml, about 0.3 μg/ml to about 8 μg/ml, about 0.4 μg/ml to about 7 μg/ml, about 0.5 μg/ml to about 6 μg/ml, about 0.6 μg/ml to about 5 μg/ml, about 0.7 μg/ml to about 4 μg/ml, about 0.8 μg/ml to about 3 μg/ml, or about 0.9 μg/ml to about 2 μg/ml. In certain embodiments, the activating agent (e.g., CD3-binding agents) is administered at a concentration of about 0.1 μg/ml, about 0.2 μg/ml, about 0.3 μg/ml, about 0.4 μg/ml, about 0.5 μg/ml, about 0.6 μg/ml, about 0.7 μg/ml, about 0.8 M, about 0.9 μg/ml, about 1 μg/ml, about 2 μg/ml, about 3 μg/ml, about 4 M, about 5 μg/ml, about 6 μg/ml, about 7 μg/ml, about 8 μg/ml, about 9 μg/ml, or about 10 μg/ml. In certain embodiments, the CD3-binding agents can be present in a concentration of 1 μg/ml.

NK cells can be activated generally using methods as described, for example, in U.S. Pat. Nos. 7,803,376, 6,949,520, 6,693,086, 8,834,900, 9,404,083, 9,464,274, 7,435,596, 8,026,097, 8,877,182; U.S. Patent Applications US2004/0058445, US2007/0160578, US2013/0011376, US2015/0118207, US2015/0037887; and PCT Patent Application WO2016/122147, each of which is incorporated herein by reference in its entirety for all purposes.

In certain embodiments, the NK based host cells can be activated by, for example and not limitation, inhibition of inhibitory receptors on NK cells (e.g., KIR2DL1, KIR2DL2/3, KIR2DL4, KIR2DL5A, KIR2DL5B, KIR3DL1, KIR3DL2, KIR3DL3, LILRB1, NKG2A, NKG2C, NKG2E or LTLRB5 receptor).

In certain embodiments, the NK based host cells can be activated by, for example and not limitation, feeder cells (e.g., native K562 cells or K562 cells that are genetically modified to express 4-1BBL and cytokines such as IL-15 or IL-21).

In other embodiments, interferons or macrophage-derived cytokines can be used to activate NK cells. For example and not limitation, such interferons include but are not limited to interferon alpha and interferon gamma, and such cytokines include but are not limited to IL-15, IL-2, IL-21.

In certain embodiments, the NK activating agent can be present in a concentration of about 0.1 to about 10 μg/ml. In certain embodiments, the NK activating agent can be present in a concentration of about 0.2 μg/ml to about 9 μg/ml, about 0.3 μg/ml to about 8 μg/ml, about 0.4 μg/ml to about 7 μg/ml, about 0.5 μg/ml to about 6 μg/ml, about 0.6 μg/ml to about 5 μg/ml, about 0.7 μg/ml to about 4 μg/ml, about 0.8 μg/ml to about 3 μg/ml, or about 0.9 μg/ml to about 2 μg/ml. In certain embodiments, the NK activating agent is administered at a concentration of about 0.1 μg/ml, about 0.2 μg/ml, about 0.3 μg/ml, about 0.4 μg/ml, about 0.5 μg/ml, about 0.6 μg/ml, about 0.7 μg/ml, about 0.8 μg/ml, about 0.9 μg/ml, about 1 μg/ml, about 2 μg/ml, about 3 μg/ml, about 4 μg/ml, about 5 μg/ml, about 6 μg/ml, about 7 μg/ml, about 8 μg/ml, about 9 μg/ml, or about 10 μg/ml. In certain embodiments, the NK activating agent can be present in a concentration of 1 μg/ml.

In certain embodiments, the activating agent is attached to a solid support such as, but not limited to, a bead, an absorbent polymer present in culture plate or well or other matrices such as, but not limited to, Sepharose or glass; may be expressed (such as in native or recombinant forms) on cell surface of natural or recombinant cell line by means known to those skilled in the art.

### Polynucleotide Transfer

In certain embodiments, the host cells are genetically modified to express a chimeric cytokine receptor described above. In certain embodiments, the host cells are further genetically modified to express a CAR, TCR, antibody, or fragment or derivative thereof, disclosed herein. The host cells can be genetically modified after stimulation/activation. In certain embodiments, the host cells are modified within 12 hours, 16 hours, 24 hours, 36 hours, or 48 hours of stimulation/activation. In certain embodiments, the cells are modified within 16 to 24 hours after stimulation/activation. In certain embodiments, the host cells are modified within 24 hours.

In order to genetically modify the host cell to express the chimeric cytokine receptor or other related molecule (e.g., CAR, TCR, antibody or fragment or derivative thereof), the polynucleotide construct must be transferred into the host cell. Polynucleotide transfer may be via viral or non-viral gene methods. Suitable methods for polynucleotide delivery for use with the current methods include any method known by those of skill in the art, by which a polynucleotide can be introduced into an organelle, cell, tissue or organism.

In some embodiments, polynucleotides are transferred to the cell in a non-viral vector. Non-viral vectors suitable for use in this invention include but are not limited to minicircle plasmids, transposon systems (e.g. Sleeping Beauty, piggyBac), or single or double stranded DNA molecules that are used as templates for homology directed repair (HDR) based gene editing.

Nucleic acid vaccines can be used to transfer polynucleotides into the host cells. Such vaccines include, but are not limited to non-viral polynucleotide vectors, “naked” DNA and RNA, and viral vectors. Methods of genetically modifying cells with these vaccines, and for optimizing the expression of genes included in these vaccines are known to those of skill in the art.

In certain embodiments, the host cells can be genetically modified by methods ordinarily used by one of skill in the art. In certain embodiments, the host cells can be transduced via retroviral transduction. References describing retroviral transduction of genes are Anderson et al., U.S. Pat. No. 5,399,346; Mann et al., Cell 33:153 (1983); Temin et al., U.S. Pat. No. 4,650,764; Temin et al., U.S. Pat. No. 4,980,289; Markowitz et al., J. Virol. 62:1120 (1988); Temin et al., U.S. Pat. No. 5,124,263; International Patent Publication No. WO 95/07358, published Mar. 16, 1995, by Dougherty et al.; and Kuo et al., Blood 82:845 (1993), each of which is incorporated herein by reference in its entirety.

One method of genetic modification includes ex vivo modification. Various methods are available for transfecting cells and tissues removed from a subject via ex vivo modification. For example, retroviral gene transfer in vitro can be used to genetically modified cells removed from the subject and the cell transferred back into the subject. See e.g., Wilson et al., Science, 244:1344-1346, 1989 and Nabel et al., Science, 244(4910):1342-1344, 1989, both of which are incorporated herein by reference in their entity. In certain embodiments, the host cells may be removed from the subject and transfected ex vivo using the polynucleotides (e.g., expression vectors) of the disclosure. In certain embodiments, the host cells obtained from the subject can be transfected or transduced with the polynucleotides (e.g., expression vectors) of the disclosure and then administered back to the subject.

Another method of gene transfer includes injection. In certain embodiments, a cell or a polynucleotide or viral vector may be delivered to a cell, tissue, or organism via one or more injections (e.g., a needle injection). Non-limiting methods of injection include injection of a composition (e.g., a saline based composition). Polynucleotides can also be introduced by direct microinjection. Non-limiting sites of injection include, subcutaneous, intradermal, intramuscular, intranodal (allows for direct delivery of antigen to lymphoid tissues). intravenous, intraprostatic, intratumor, intralymphatic (allows direct administration of DCs) and intraperitoneal. It is understood that proper site of injection preparation is necessary (e.g., shaving of the site of injection to observe proper needle placement).

Electroporation is another method of polynucleotide delivery. See e.g., Potter et al., (1984) Proc. Nat'l Acad. Sci. USA, 81, 7161-7165 and Tur-Kaspa et al., (1986) Mol. Cell Biol., 6, 716-718, both of which are incorporated herein in their entirety for all purposes. Electroporation involves the exposure of a suspension of cells and DNA to a high-voltage electric discharge. In certain embodiments, cell wall-degrading enzymes, such as pectin-degrading enzymes, can be employed to render the host cells more susceptible to genetic modification by electroporation than untreated cells. See e.g., U.S. Pat. No. 5,384,253, incorporated herein by reference in its entirety for all purposes.

In vivo electroporation involves a basic injection technique in which a vector is injected intradermally in a subject. Electrodes then apply electrical pulses to the intradermal site causing the cells localized there (e.g., resident dermal dendritic cells), to take up the vector. These tumor antigen-expressing dendritic cells activated by local inflammation can then migrate to lymph-nodes.

Methods of electroporation for use with this invention include, for example, Sardesai, N. Y., and Weiner, D. B., Current Opinion in Immunotherapy 23:421-9 (2011) and Ferraro, B. et al., Human Vaccines 7:120-127 (2011), both of which are hereby incorporated by reference herein in their entirety for all purposes.

Additional methods of polynucleotide transfer include liposome-mediated transfection (e.g., polynucleotide entrapped in a lipid complex suspended in an excess of aqueous solution. See e.g., Ghosh and Bachhawat, (1991) In: Liver Diseases, Targeted Diagnosis and Therapy Using Specific Receptors and Ligands. pp. 87-104). Also contemplated is a polynucleotide complexed with Lipofectamine, or Superfect); DEAE-dextran (e.g., a polynucleotide is delivered into a cell using DEAE-dextran followed by polyethylene glycol. See e.g., Gopal, T. V., Mol Cell Biol. 1985 May; 5(5):1188-90); calcium phosphate (e.g., polynucleotide is introduced to the cells using calcium phosphate precipitation. See e.g., Graham and van der Eb, (1973) Virology, 52, 456-467; Chen and Okayama, Mol. Cell Biol., 7(8):2745-2752, 1987), and Rippe et al., Mol. Cell Biol., 10:689-695, 1990); sonication loading (introduction of a polynucleotide by direct sonic loading. See e.g., Fechheimer et al., (1987) Proc. Nat'l Acad. Sci. USA, 84, 8463-8467); microprojectile bombardment (e.g., one or more particles may be coated with at least one polynucleotide and delivered into cells by a propelling force. See e.g., U.S. Pat. Nos. 5,550,318; 5,538,880; 5,610,042; and PCT Application WO 94/09699; Klein et al., (1987) Nature, 327, 70-73, Yang et al., (1990) Proc. Nat'l Acad. Sci. USA, 87, 9568-9572); and receptor-mediated transfection (e.g., selective uptake of macromolecules by receptor-mediated endocytosis that will be occurring in a target cell using cell type-specific distribution of various receptors. See e.g., Wu and Wu, (1987) J. Biol. Chem., 262, 4429-4432; Wagner et al., Proc. Natl. Acad. Sci. USA, 87(9):3410-3414, 1990; Perales et al., Proc. Natl. Acad. Sci. USA, 91:4086-4090, 1994; Myers, EPO 0273085; Wu and Wu, Adv. Drug Delivery Rev., 12:159-167, 1993; Nicolau et al., (1987) Methods Enzymol., 149, 157-176), each reference cited here is incorporated by reference in their entirety for all purposes.

In further embodiments, host cells are genetically modified using gene editing with homology-directed repair (HDR). Homology-directed repair (HDR) is a mechanism used by cells to repair double strand DNA breaks. In HDR, a donor polynucleotide with homology to the site of the double strand DNA break is used as a template to repair the cleaved DNA sequence, resulting in the transfer of genetic information from the donor polynucleotide to the DNA. As such, new nucleic acid material may be inserted or copied into a target DNA cleavage site. Double strand DNA breaks in host cells may be induced by a site-specific nuclease. The term “site-specific nuclease” as used herein refers to a nuclease capable of specifically recognizing and cleaving a nucleic acid (DNA or RNA) sequence. Suitable site-specific nucleases for use in the present invention include, but are not limited to, RNA-guided endonuclease (e.g., CRISPR-associated (Cas) proteins), zinc finger nuclease, a TALEN nuclease, or mega-TALEN nuclease. For example, a site-specific nuclease (e.g., a Cas9+guide RNA) capable of inducing a double strand break in a target DNA sequence is introduced to a host cell, along with a donor polynucleotide encoding a chimeric cytokine receptor of the present disclosure and optionally an additional protein (e.g., CAR, TCR, antibody, or fragment or derivative thereof).

### Expansion/Proliferation

After the host cells are activated and transduced, the cells are cultured to proliferate. T-cells may be cultured for at least 1, 2, 3, 4, 5, 6, or 7 days, at least 2 weeks, at least 1, 2, 3, 4, 5, or 6 months or more with 1, 2, 3, 4, 5, 6, 7, 8, 9, or 10 or more rounds of expansion.

Agents that can be used for the expansion of T-cells can include interleukins, such as IL-2, IL-7, IL-15, or IL-21 (see for example Cornish et al. 2006, Blood. 108(2):600-8, Bazdar and Sieg, 2007, Journal of Virology, 2007, 81(22):12670-12674, Battalia et al, 2013, Immunology, 139(1):109-120). Other illustrative examples for agents that may be used for the expansion of T-cells are agents that bind to CD8, CD45 or CD90, such as αCD8, αCD45 or αCD90 antibodies. Illustrative examples of T-cell population including antigen-specific T-cells, T helper cells, cytotoxic T-cells, memory T-cell (an illustrative example of memory T-cells are CD62L|CD8| specific central memory T-cells) or regulatory T-cells (an illustrative example of Treg are CD4+ CD25+ CD45RA+ Treg cells).

Additional agents that can be used to expand T lymphocytes includes methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041, each of which is incorporated herein by reference in its entirety.

In certain embodiments, the agent(s) used for expansion (e.g., IL-2) are administered at about 20 units/ml to about 200 units/ml. In certain embodiments, the agent(s) used for expansion (e.g., IL-2) are administered at about 25 units/ml to about 190 units/ml, about 30 units/ml to about 180 units/ml, about 35 units/ml to about 170 units/ml, about 40 units/ml to about 160 units/ml, about 45 units/ml to about 150 units/ml, about 50 units/ml to about 140 units/ml, about 55 units/ml to about 130 units/ml, about 60 units/ml to about 120 units/ml, about 65 units/ml to about 110 units/ml, about 70 units/ml to about 100 units/ml, about 75 units/ml to about 95 units/ml, or about 80 units/ml to about 90 units/ml. In certain embodiments, the agent(s) used for expansion (e.g., IL-2) are administered at about 20 units/ml, about 25 units/ml, about 30 units/ml, 35 units/ml, 40 units/ml, 45 units/ml, about 50 units/ml, about 55 units/ml, about 60 units/ml, about 65 units/ml, about 70 units/ml, about 75 units/ml, about 80 units/ml, about 85 units/ml, about 90 units/ml, about 95 units/ml, about 100 units/ml, about 105 units/ml, about 110 units/ml, about 115 units/ml, about 120 units/ml, about 125 units/ml, about 130 units/ml, about 135 units/ml, about 140 units/ml, about 145 units/ml, about 150 units/ml, about 155 units/ml, about 160 units/ml, about 165 units/ml, about 170 units/ml, about 175 units/ml, about 180 units/ml, about 185 units/ml, about 190 units/ml, about 195 units/ml, or about 200 units/ml. In certain embodiments, the agent(s) used for expansion (e.g., IL-2) are administered at about 5 mg/ml to about 10 ng/ml. In certain embodiments, the agent(s) used for expansion (e.g., IL-2) are administered at about 5.5 ng/ml to about 9.5 ng/ml, about 6 ng/ml to about 9 ng/ml, about 6.5 ng/ml to about 8.5 ng/ml, or about 7 ng/ml to about 8 ng/ml. In certain embodiments, the agent(s) used for expansion (e.g., IL-2) are administered at about 5 ng/ml, 6 ng/ml, 7 ng/ml, 8 ng/ml, 9, ng/ml, or 10 ng/ml.

After the host cells are activated and transduced, the cells are cultured to proliferate. NK cells may be cultured for at least 1, 2, 3, 4, 5, 6, or 7 days, at least 2 weeks, at least 1, 2, 3, 4, 5, or 6 months or more with 1, 2, 3, 4, 5, 6, 7, 8, 9, or 10 or more rounds of expansion.

Agents that can be used for the expansion of natural killer cells can include agents that bind to CD16 or CD56, such as for example αCD16 or αCD56 antibodies. In certain embodiments, the binding agent includes antibodies (see for example Hoshino et al, Blood. 1991 Dec. 15; 78(12):3232-40.). Other agents that may be used for expansion of NK cells may be IL-15 (see for example Vitale et al. 2002. The Anatomical Record. 266:87-92, which is hereby incorporated by reference in its entirety for all purposes).

Conditions appropriate for T-cell culture include an appropriate media (e.g., Minimal Essential Media (MEM), RPMI Media 1640, Lonza RPMI 1640, Advanced RPMI, Clicks, AIM-V, DMEM, a-MEM, F-12, TexMACS, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion).

Examples of other additives for host cell expansion include, but are not limited to, surfactant, piasmanate, pH buffers such as HEPES, and reducing agents such as N-acetyl-cysteine and 2-mercaptoethanol, Antibiotics (e.g., penicillin and streptomycin), are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

In certain embodiments, host cells of the present disclosure may be modified such that the expression of an endogenous TCR, MHC molecule, or other immunogenic molecule is decreased or eliminated. When allogeneic cells are used, rejection of the therapeutic cells may be a concern as it may cause serious complications such as the graft-versus-host disease (GvHD). Although not wishing to be bound by theory, immunogenic molecules (e.g., endogenous TCRs and/or MHC molecules) are typically expressed on the cell surface and are involved in self vs non-self discrimination. Decreasing or eliminating the expression of such molecules may reduce or eliminate the ability of the therapeutic cells to cause GvHD.

In certain embodiments, expression of an endogenous TCR in the host cells is decreased or eliminated. In a particular embodiment, expression of an endogenous TCR (e.g., αβ TCR) in the host cells is decreased or eliminated. Expression of the endogenous TCR may be decreased or eliminated by disrupting the TRAC locus, TCR beta constant locus, and/or CD3 locus. In certain embodiments, expression of an endogenous TCR may be decreased or eliminated by disrupting one or more of the TRAC, TRBC1, TRBC2, CD3E, CD3G, and/or CD3D locus.

In certain embodiments, expression of one or more endogenous MHC molecules in the host cells is decreased or eliminated. Modified MHC molecule may be an MHC class I or class II molecule. In certain embodiments, expression of an endogenous MHC molecule may be decreased or eliminated by disrupting one or more of the MHC, β2M, TAP1, TAP2, CIITA, RFX5, RFXAP and/or RFXANK locus.

Expression of an endogenous TCR, an MHC molecule, and/or any other immunogenic molecule in the host cell can be disrupted using genome editing techniques such as Clustered regularly interspaced short palindromic repeats (CRISPR)/Cas, zinc finger nucleases (ZFNs), transcription activator-like effector nucleases (TALENs), and Meganucleases. These genome editing methods may disrupt a target gene by entirely knocking out all of its output or partially knocking down its expression. In a particular embodiment, expression of the endogenous TCR, an MHC molecule and/or any other immunogenic molecule in the host cell is disrupted using the CRISPR/Cas technique.

### Pharmaceutical Compositions

In some embodiments, the compositions comprise one or more polypeptides of the chimeric cytokine receptor and other related molecules (e.g., CARs, TCRs or antibodies, or fragments or derivatives thereof), polynucleotides, vectors comprising same, and cell compositions, as disclosed herein. Compositions of the present disclosure include, but are not limited to pharmaceutical compositions.

In some embodiments, the pharmaceutical compositions comprise any of the host cells disclosed herein and a pharmaceutically acceptable carrier and/or excipient.

In one aspect, the present disclosure provides a pharmaceutical composition comprising a polynucleotide or a recombinant vector encoding a chimeric cytokine receptor described herein, and a pharmaceutically accepted carrier and/or excipient.

In another aspect, the present disclosure provides pharmaceutical composition comprising the modified host cells comprising a chimeric cytokine receptor described herein and a pharmaceutically acceptable carrier and/or excipient.

Examples of pharmaceutical carriers include but are not limited to sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions.

Compositions comprising modified host cells disclosed herein may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

Compositions comprising modified host cells disclosed herein may comprise one or more of the following: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose.

In some embodiments, the compositions are formulated for parenteral administration, e.g., intravascular (intravenous or intraarterial), intraperitoneal, intratumoral, intraventricular, intrapleural or intramuscular administration. The parenteral preparation can be enclosed in ampoules, disposable syringes or multiple dose vials made of glass or plastic. An injectable pharmaceutical composition is preferably sterile. In some embodiments, the composition is reconstituted from a lyophilized preparation prior to administration.

In some embodiments, the modified host cells may be mixed with substances that adhere or penetrate then prior to their administration, e.g., but not limited to, nanoparticles.

### Therapeutic Methods

In one aspect, the present disclosure provides a method of enhancing effector function of an immune cell, comprising genetically modifying the cell with the polynucleotide or the recombinant vector encoding a chimeric cytokine receptor. In some embodiments, the immune cell expresses a chimeric antigen receptor (CAR), an antigen specific T cell receptor (TCR) and/or an antibody, or fragment or derivative thereof.

In some embodiments, the present disclosure provides a method of enhancing effector function an immune cell, wherein the immune cell expresses a chimeric antigen receptor (CAR), comprising genetically modifying the cell with the polynucleotide disclosed herein or the recombinant vector disclosed herein.

In some embodiments, the effector function is one or more of expansion, persistence, and/or anti-tumor activity.

The terms “expand” or “expansion” when used in relation to an immune cell refer to the ability of the immune cell to undergo cellular proliferation (i.e., to increase the number of cells). The terms used herein encompass both in vivo and in vitro immune cell expansion.

The terms “persist” or “persistence” when used in relation to an immune cell refer to the ability of the immune cell (and/or its progenies) to be maintained in a recipient (e.g., a subject) for a period of time. The terms used herein encompass both in vivo and in vitro immune cell persistence.

The term “tumor killing activity” as used herein refers to the ability of an immune cell to inhibit tumor growth and/or to kill the tumor cells (e.g., cancer cells).

In one aspect, the present disclosure provides a method of treating a disease comprising administering to the subject an effective amount of the host cell comprising a chimeric cytokine receptor described herein, or the pharmaceutical composition comprising the host cells. In some embodiments, the disease is cancer such as, but not limited to, a solid tumor. In some embodiments, the disease is infection. In some embodiments, the disease is an autoimmune disease.

In one aspect, the present disclosure provides a method for treating a tumor in a subject in need thereof. A therapeutically effective amount of the modified host cells comprising a chimeric cytokine receptor described herein or the pharmaceutical composition comprising the host cells is administered to the subject.

The term “tumor” refers to a benign or malignant abnormal growth of tissue. The term “tumor” includes cancer. Examples of tumors are, but not limited to, the soft tissue tumors (e.g., lymphomas), and tumors of the blood and blood-forming organs (e.g., leukemias), and solid tumors, which is one that grows in an anatomical site outside the bloodstream (e.g., carcinomas). Examples of cancer include, but are not limited to, carcinoma, lymphoma, blastoma, sarcoma (e.g., osteosarcoma or rhabdomyosarcoma), and leukemia or lymphoid malignancies. More particular examples of such cancers include squamous cell cancer (e.g., epithelial squamous cell cancer), adenosquamous cell carcinoma, lung cancer (e.g., including small-cell lung cancer, non-small cell lung cancer, adenocarcinoma of the lung, squamous carcinoma of the lung), cancer of the peritoneum, hepatocellular cancer, gastric or stomach cancer (e.g., including gastrointestinal cancer, pancreatic cancer), cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, primary or metastatic melanoma, multiple myeloma and B-cell lymphoma, non-Hodgkin's lymphoma, Hodgkin's lymphoma, brain (e.g., high grade glioma, diffuse pontine glioma, ependymoma, neuroblastoma, or glioblastoma), as well as head and neck cancer, and associated metastases. Additional examples of tumors can be found in The Merck Manual of Diagnosis and Therapy, 19th Edition, § on Hematology and Oncology, published by Merck Sharp & Dohme Corp., 2011 (ISBN 978-0-911910-19-3); The Merck Manual of Diagnosis and Therapy, 20th Edition, § on Hematology and Oncology, published by Merck Sharp & Dohme Corp., 2018 (ISBN 978-0-911-91042-1) (2018 digital online edition at internet website of Merck Manuals); and SEER Program Coding and Staging Manual 2016, each of which are incorporated by reference in their entirety for all purposes.

In some embodiments, the cancer being treated by methods of the present invention is a HER2 positive cancer. In some embodiments, the HER2 positive cancer is brain, breast, stomach, ovary, uterine serous endometrial carcinoma, colon, bladder, lung, uterine cervix, head and neck, sarcoma, bone tumors, or esophagus cancer.

In some embodiments, the cancer being treated by methods of the present invention is a EphA2 positive cancer. In some embodiments, the EphA2 positive cancer is breast, prostate, urinary bladder, skin, lung, ovary, sarcoma, bone tumors or brain cancer.

In some embodiments, the cancer being treated by methods of the present invention is a B7-H3 positive cancer. In some embodiments, the B7-H3 positive cancer is osteosarcoma, rhabdomyosarcoma, Ewing sarcoma and other Ewing sarcoma family of tumors, neuroblastoma, ganglioneuroblastoma, desmoplastic small round cell tumor, malignant peripheral nerve sheath tumor, synovial sarcoma, undifferentiated sarcoma, adrenocortical carcinoma, hepatoblastoma, Wilms tumor, rhabdoid tumor, high grade glioma (glioblastoma multiforme), medulloblastoma, astrocytoma, glioma, ependymoma, atypical teratoid rhabdoid tumor, meningioma, craniopharyngioma, primitive neuroectodermal tumor, diffuse intrinsic pontine glioma and other brain tumors, acute myeloid leukemia, multiple myeloma, lung cancer, mesothelioma, breast cancer, bladder cancer, gastric cancer, prostate cancer, colorectal cancer, endometrial cancer, cervical cancer, renal cancer, esophageal cancer, ovarian cancer, pancreatic cancer, hepatocellular carcinoma and other liver cancers, head and neck cancers, leiomyosarcoma, and melanoma.

In some embodiments, the therapeutic method of the present disclosure includes one or more of the following steps: a) isolating immune cells (e.g., T cells or NK cells) from the subject or donor; b) modifying immune cells (e.g., T cells or NK cells) ex vivo with the polynucleotide or the recombinant vector encoding a chimeric cytokine receptor described herein; c) optionally modifying the immune cells (e.g., T cells or NK cells) ex vivo to express a chimeric antigen receptor (CAR), an antigen specific T cell receptor (TCR) and/or an antibody, or fragment of derivative thereof, said CAR, TCR or antibody specifically binds an antigen associated with said disease; d) optionally, expanding and/or activating the modified the immune cells (e.g., T cells or NK cells) before, after and/or during step b) or c); and e) introducing a therapeutically effective amount of the modified immune cells (e.g., T cells or NK cells) into the subject. In some embodiments, the immune cells express GR-CSF upon activation. In some embodiments, the immune cell is an αβ TCR T cell, a γδ T cell, or an iNKT cell.

In some embodiments, the therapeutic method of the present disclosure may comprise one or more of the following steps: a) isolating T cells or NK cells from the subject or donor; b) modifying said T cells or NK cells ex vivo with a polynucleotide encoding a chimeric cytokine receptor described herein or the recombinant vector encoding said polynucleotide; c) optionally modifying said T cells or NK cells ex vivo to express a chimeric antigen receptor (CAR) that binds an antigen associated with said disease; d) optionally, expanding and/or activating the modified T cells or NK cells before, after and/or during step b) or c); and e) introducing a therapeutically effective amount of the modified T cells or NK cells into the subject.

In some embodiments, the modified host cell is an autologous cell. In some embodiments, the modified host cell is an allogeneic cell. In cases where the host cell is isolated from a donor, the method may further include a method to prevent graft vs host disease (GVHD) and the host cell rejection.

In some embodiments, the modified host cells may also express a CD20 polypeptide as a safety switch. Accordingly, the method may further include administering an anti-CD20 antibody to the subject for removal of the isolated host cells. The anti-CD20 antibody is administered in an amount effective for sufficient removal of the isolated host cells from the subject. In some embodiments, the anti-CD20 antibody is administered in an amount effective for removal of more than 50% of the isolated host cells from the subject. For example, the anti-CD20 antibody may be administered in an amount effective for removal of more than 55%, more than 60%, more than 65%, more than 70%, more than 75%, more than 80%, more than 85%, more than 90%, more than 95%, more than 98%, more than 99%, or about 100% of the isolated host cells from the subject. The anti-CD20 antibody may be administered in an amount effective for removal of about 50% to about 70%, about 60% to about 80%, about 70% to about 90%, or about 80% to about 100% of the isolated host cells from the subject.

Non-limiting examples of anti-CD20 antibodies that can be used for removal the isolated host cells include Rituximab, Ibritumomab tiuxetan, Tositumomab, Ofatumumab, Ocrelizumab, TRU-015, Veltuzumab, AME-133v, PRO131921, and Obinutuzumab. In some embodiments, the anti-CD20 antibody is Rituximab.

In some embodiments of any of the therapeutic methods described above, the composition is administered in a therapeutically effective amount. The dosages of the composition administered in the methods of the invention will vary widely, depending upon the subject's physical parameters, the frequency of administration, the manner of administration, the clearance rate, and the like. The initial dose may be larger, and might be followed by smaller maintenance doses. The dose may be administered as infrequently as weekly or biweekly, or fractionated into smaller doses and administered daily, semi-weekly, etc., to maintain an effective dosage level. It is contemplated that a variety of doses will be effective to achieve in vivo persistence of modified host cells. It is also contemplated that a variety of doses will be effective to improve in vivo effector function of modified host cells.

In some embodiments, composition comprising the modified host cells manufactured by the methods described herein may be administered at a dosage of 10² to 10¹⁰ cells/kg body weight, 10⁵ to 10⁹ cells/kg body weight, 10⁵ to 10⁸ cells/kg body weight, 10⁵ to 10⁷ cells/kg body weight, 10⁷ to 10⁹ cells/kg body weight, or 10⁷ to 10⁸ cells/kg body weight, including all integer values within those ranges. The number of modified host cells will depend on the therapeutic use for which the composition is intended for.

Modified host cells may be administered multiple times at dosages listed above. The modified host cells may be allogeneic, syngeneic, xenogeneic, or autologous to the patient undergoing therapy.

The compositions and methods described in the present disclosure may be utilized in conjunction with other types of therapy for tumors, such as chemotherapy, surgery, radiation, gene therapy, and so forth.

It is also contemplated that when used to treat various diseases/disorders, the compositions and methods of the present disclosure can be utilized with other therapeutic methods/agents suitable for the same or similar diseases/disorders. Such other therapeutic methods/agents can be co-administered (simultaneously or sequentially) to generate additive or synergistic effects. Suitable therapeutically effective dosages for each agent may be lowered due to the additive action or synergy.

In some embodiments of any of the above therapeutic methods, the method further comprises administering to the subject one or more additional compounds selected from the group consisting of immuno-suppressives, biologicals, probiotics, prebiotics, and cytokines (e.g., GM-CSF, IFN or IL-2).

In some embodiments, the method described herein further comprises providing exogenous GM-CSF, in addition to the GM-CSF produced by the immune cells, to enhance the function of immune cells expressing a chimeric cytokine receptor of the present disclosure. Exogenous GM-CSF may be provided by, for example and not limitation, i) injection of the FDA-approved GM-CSF drug Sargramostin (Leukine™) or ii) the use of nonviral or viral vectors to express GM-CSF (e.g., FDA-approved GM-CSF expressing oncolytic virus talimogene laherparepvec [TVEC, Imlygic™]). These drugs could be given before, with, or after the administration (e.g., infusion) of the immune cells expressing a chimeric cytokine receptor of the present disclosure to patients.

As a non-limiting example, the invention can be combined with other therapies that block inflammation (e.g., via blockage of IL-1, IFNα/β, IL-6, TNF, IL-23, etc.).

The methods and compositions of the invention can be combined with other immunomodulatory treatments such as, e.g., therapeutic vaccines (including but not limited to GVAX, DC-based vaccines, etc.), checkpoint inhibitors (including but not limited to agents that block CTLA4, PD1, LAG3, TIM3, etc.) or activators (including but not limited to agents that enhance 4-1BB, OX40, etc.). The methods of the invention can be also combined with other treatments that possess the ability to modulate NKT function or stability, including but not limited to CD1d, CD1d-fusion proteins, CD1d dimers or larger polymers of CD1d either unloaded or loaded with antigens, CD1d-chimeric antigen receptors (CD1d-CAR), or any other of the five known CD1 isomers existing in humans (CD1a, CD1b, CD1c, CD1e). The methods of the invention can also be combined with other treatments such as midostaurin, enasidenib, or a combination thereof.

Therapeutic methods of the invention can be combined with additional immunotherapies and therapies. For example, when used for treating tumors, the compositions of the invention can be used in combination with conventional therapies, such as, e.g., surgery, radiotherapy, chemotherapy or combinations thereof, depending on type of the tumor, patient condition, other health issues, and a variety of factors. In certain aspects, other therapeutic agents useful for combination tumor therapy with the inhibitors of the invention include anti-angiogenic agents. Many anti-angiogenic agents have been identified and are known in the art, including, e.g., TNP-470, platelet factor 4, thrombospondin-1, tissue inhibitors of metalloproteases (TIMP1 and TIMP2), prolactin (16-Kd fragment), angiostatin (38-Kd fragment of plasminogen), endostatin, bFGF soluble receptor, transforming growth factor beta, interferon alpha, soluble KDR and FLT-1 receptors, placental proliferin-related protein, as well as those listed by Carmeliet and Jain (2000). In one embodiment, the modified host cells of the invention can be used in combination with a VEGF antagonist or a VEGF receptor antagonist such as anti-VEGF antibodies, VEGF variants, soluble VEGF receptor fragments, aptamers capable of blocking VEGF or VEGFR, neutralizing anti-VEGFR antibodies, inhibitors of VEGFR tyrosine kinases and any combinations thereof (e.g., anti-hVEGF antibody A4.6.1, bevacizumab or ranibizumab).

Non-limiting examples of chemotherapeutic compounds which can be used in combination treatments of the present disclosure include, for example, aminoglutethimide, amsacrine, anastrozole, asparaginase, azacitidine, bcg, bicalutamide, bleomycin, buserelin, busulfan, campothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, decitabine, dienestrol, diethylstilbestrol, docetaxel, doxorubicin, epirubicin, estradiol, estramnustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

These chemotherapeutic compounds may be categorized by their mechanism of action into, for example, following groups: anti-metabolites/anti-tumor agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethyhnelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (e.g., TNP-470, genistein, bevacizumab) and growth factor inhibitors (e.g., fibroblast growth factor (FGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers and caspase activators; and chromatin disruptors.

In various embodiments of the methods described herein, the subject is a human. The subject may be a juvenile or an adult, of any age or sex.

In accordance with the present invention there may be numerous tools and techniques within the skill of the art, such as those commonly used in molecular biology, pharmacology, and microbiology. Such tools and techniques are described in detail in e.g., Sambrook et al. (2001) Molecular Cloning: A Laboratory Manual. 3rd ed. Cold Spring Harbor Laboratory Press: Cold Spring Harbor, New York; Ausubel et al. eds. (2005) Current Protocols in Molecular Biology. John Wiley and Sons, Inc.: Hoboken, NJ; Bonifacino et al. eds. (2005) Current Protocols in Cell Biology. John Wiley and Sons, Inc.: Hoboken, NJ; Coligan et al. eds. (2005) Current Protocols in Immunology, John Wiley and Sons, Inc.: Hoboken, NJ; Coico et al. eds. (2005) Current Protocols in Microbiology, John Wiley and Sons, Inc.: Hoboken, NJ; Coligan et al. eds. (2005) Current Protocols in Protein Science, John Wiley and Sons, Inc.: Hoboken, NJ; and Enna et al. eds. (2005) Current Protocols in Pharmacology, John Wiley and Sons, Inc.: Hoboken, NJ.

### EXAMPLES

The following examples are provided to further describe some of the embodiments disclosed herein. The examples are intended to illustrate, not to limit, the disclosed embodiments.

### Example 1. 2X Leucine Zipper IL-2 Receptor (zipIL-2R) Activates STAT5 and Improves T Cell Survival

Various approaches have been utilized to enhance the expansion and persistence of chimeric antigen receptor (CAR) T cells in order to improve the treatment of hematologic malignancies and solid tumors. One such approach is the modulation of cytokine signaling, for example by lymphodepletion to increase the serum concentration of cytokines such as interleukin-15 (IL-15) or transgenic expression of cytokines by chimeric antigen receptor T cells (CAR T cells). The present approach to engineer constitutive active forms of cytokine receptors via forced dimerization of leucine zipper motifs (zipReceptors) (FIG. 2A) is designed to provide proliferative and survival signals to CAR T cells without the toxicity associated with transgenic expression of pleiotropic cytokines.

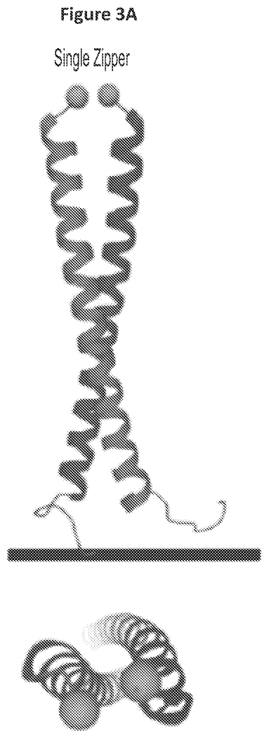

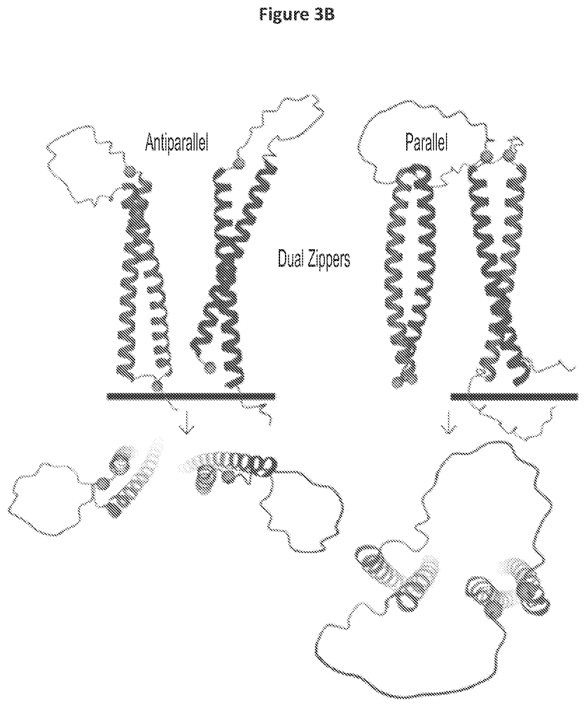

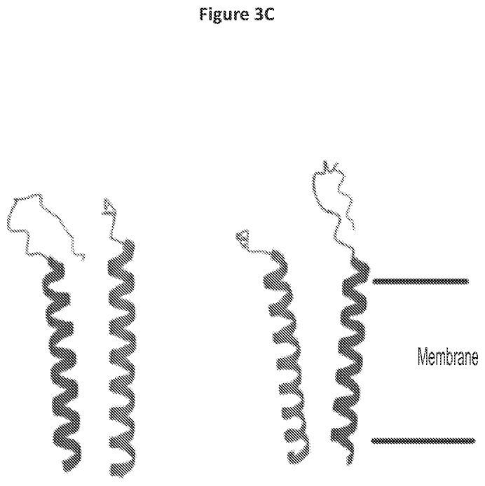

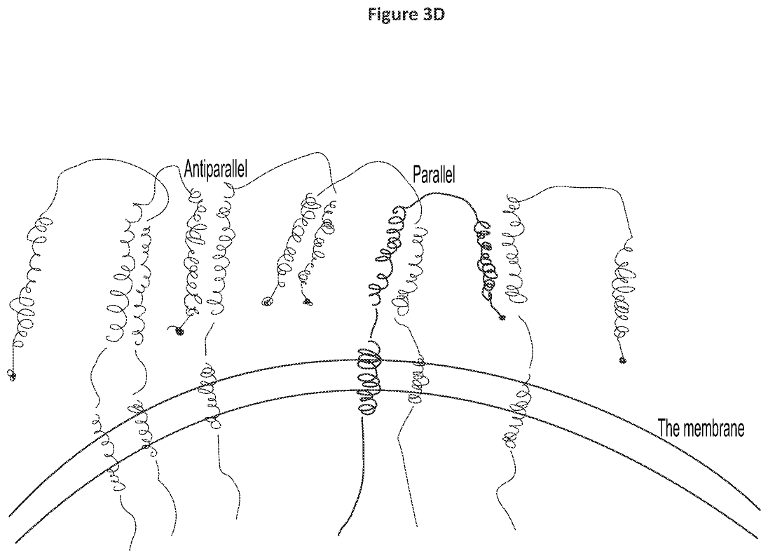

Initial designs of leucine zipper-based IL-2 receptor (IL-2R) chains (IL-2Rβ and IL-2Rγ) with either one (1X) or two pairs (2X) of leucine zipper motifs were efficiently expressed by y retrovirally-transduced human T cells (representative flow cytometry plots in FIG. 2B and quantified in FIG. 2C). However, IL-2R chains with two pairs of leucine zipper motifs (zipIL-2R.2X) robustly activated signal transducer and activator of transcription 5 (STAT5) (representative flow cytometry plots in FIG. 2B and quantified in FIG. 2C). This result was unexpected since previous publications had shown activation of CAR and cytokine signaling pathways with only one pair of leucine zipper motifs [31,32]. Structural modeling of the zipIL-2R.1X and zipIL-2R.2X receptor chains demonstrated that the zipIL-2R.1X chains can form heterodimers, whereas the zipIL-2R.2X receptor chains have the ability to form oligomers (FIG. 3). To demonstrate the functional activity of zipReceptors, transduced T cells were starved of cytokines for seven days and cell viability was determined by cell surface expression of Annexin V and exclusion of a cell viability dye via flow cytometry. While most non-transduced (NT) T cells were dead (Annexin V+ and viability dye+), a majority of zipIL-2R.2X expressing T cells were alive (Annexin V− and viability dye−) (representative flow cytometry plots in FIG. 2D and quantified in FIG. 2E). In contrast, the majority of IL-2Rβ.1X+IL-2Rγ.1X-expressing T cells were dead, consistent with limited STAT5 activation by these receptors.

### Example 2. Two Leucine Zipper Domains Enable Oligomerization of zipReceptors

AlphaFold [33]protein structure prediction was used to develop a structural explanation for the observed differences in signaling between 1X and 2X zipReceptors. Modeling of IL-2Rβ and IL-2Rγ with one or two pairs of leucine zipper motifs revealed structures with high confidence in prediction of transmembrane and leucine zipper domains. While 1X zipReceptors were only predicted to form dimeric structures with leucine zippers interacting in a parallel orientation (FIG. 3A), 2X leucine zipper domains were predicted to interact in parallel or antiparallel alignment (FIG. 3B). Because the transmembrane domains were not predicted to form favorable interactions, they were unlikely to contribute to dimerization or signaling (FIG. 3C). Therefore, the observed activity of 2X zipReceptors may be attributed to the formation of higher order complexes via interaction of 2X leucine zipper motifs in parallel and antiparallel aligns as suggested by the domain swapping model [34](FIG. 3D).

### Example 3. ZipIL-2R Enhances B7-H3-CAR T Cell Antitumor Activity In Vitro without Altering Antigen Specificity

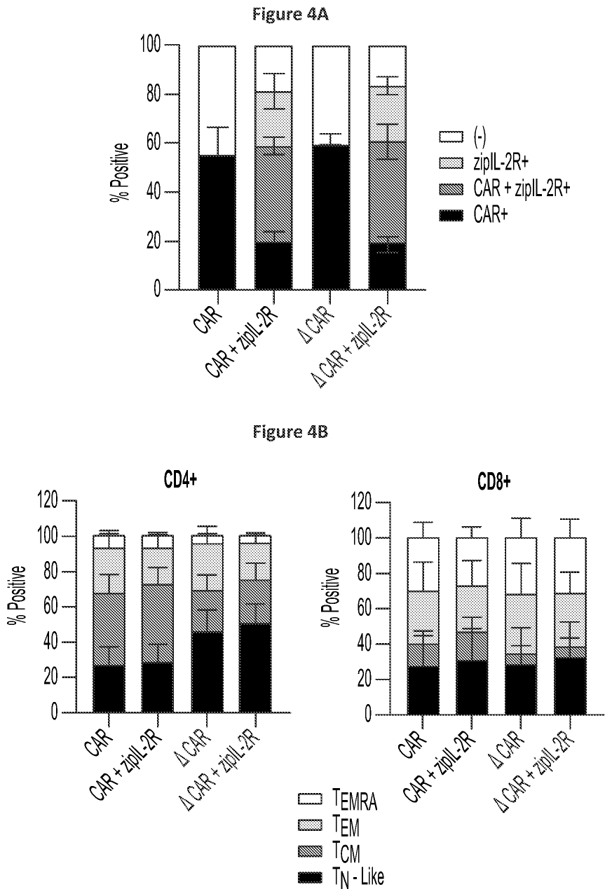

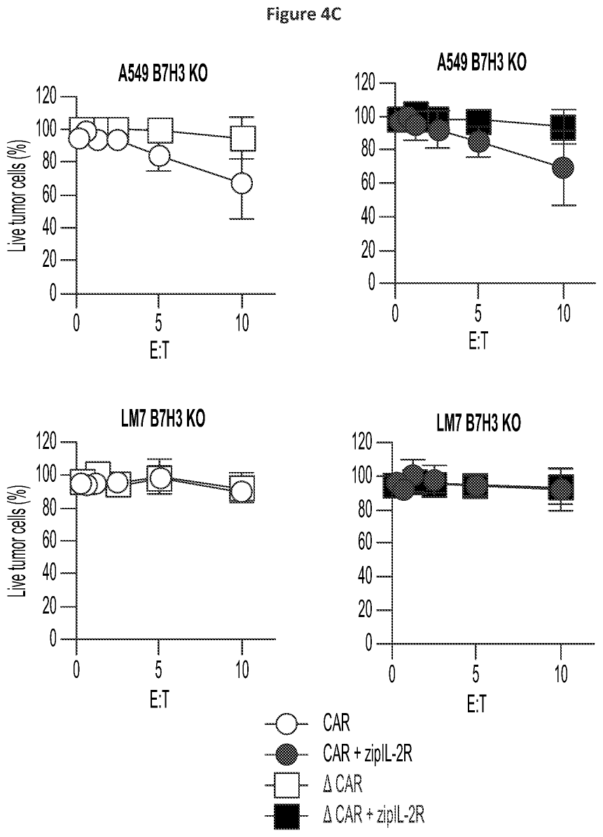

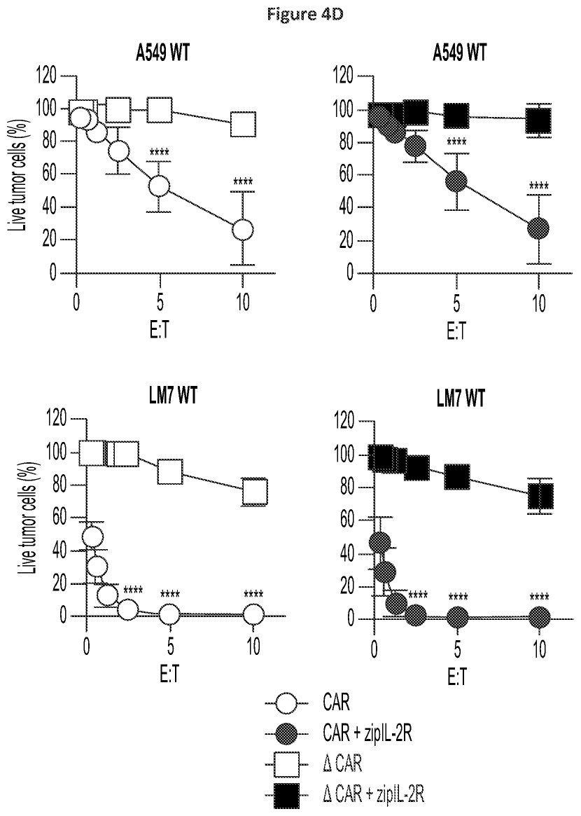

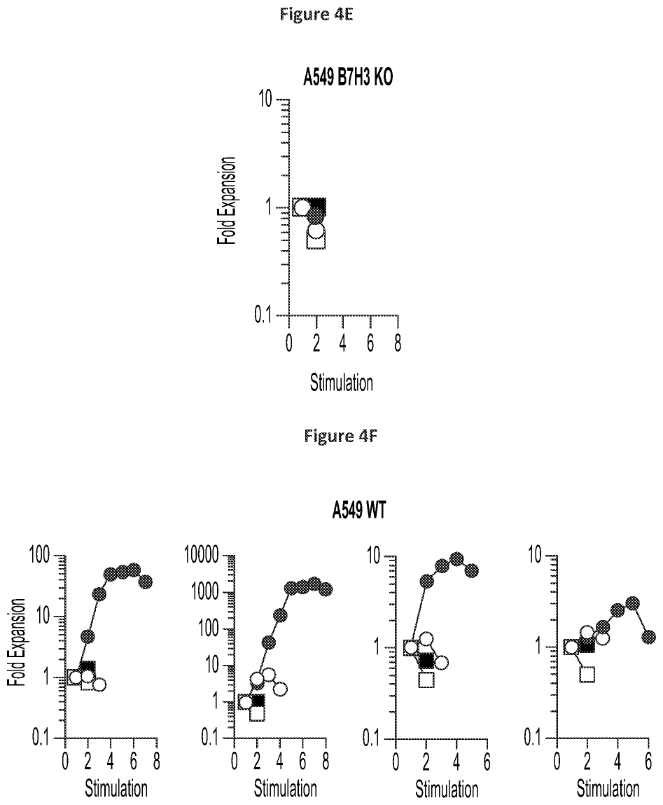

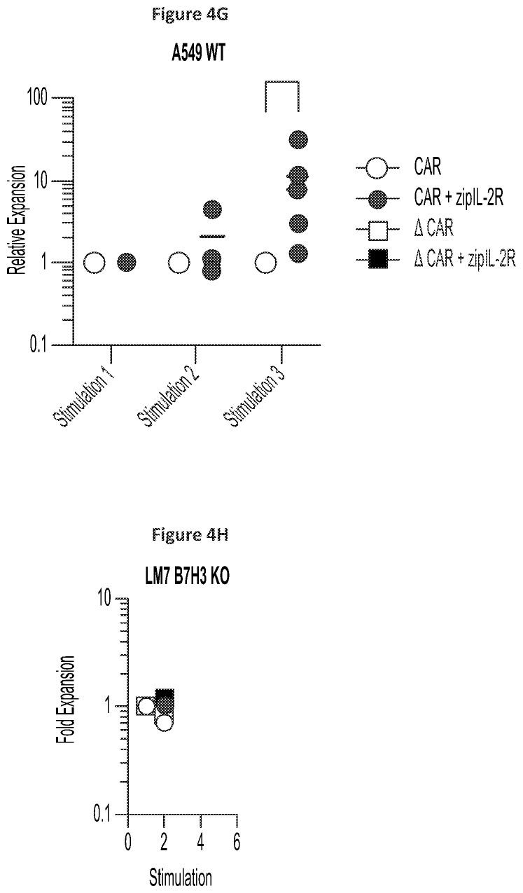

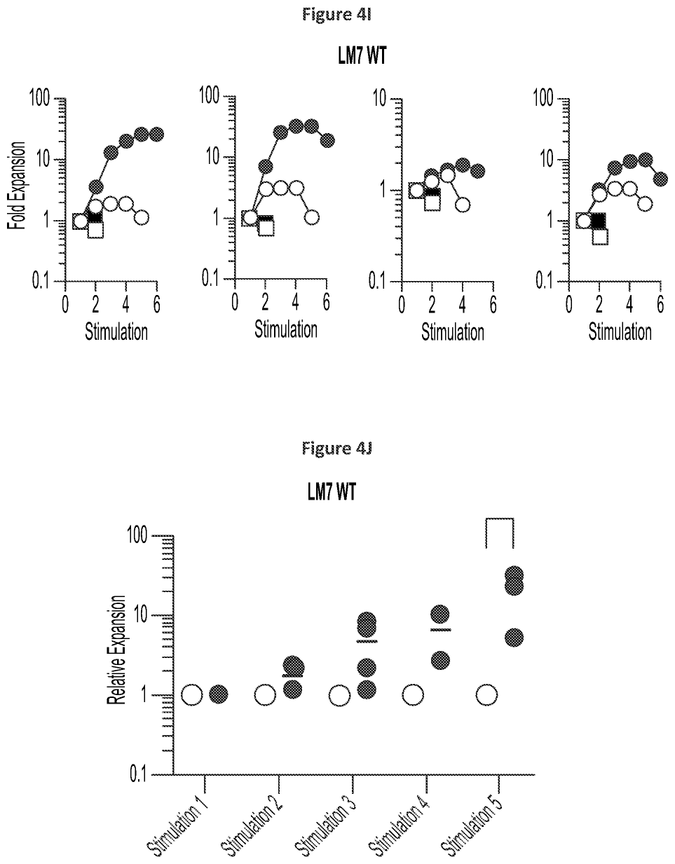

T cells expressing a functional B7-H3-CAR (CAR) or a non-functional B7-H3-CAR (ΔCAR) were generated by sequential transduction with CAR or ΔCAR and zipIL-2R viral vector (FIG. 4A) without altering T cell subsets (FIG. 4B). To measure cytotoxicity and antigen specificity of CAR T cells, a 24-hour MTS assay was performed with B7-H3 positive (A549 WT and LM7 WT) and B7-H3 negative (A549 B7-H3 KO and LM7 B7-H3 KO) cell lines (FIG. 4C). Both CAR and CAR+zipIL-2R T cells killed B7-H3-positive cells at different effector cell to target cell ratios (E:T), while ΔCAR and ΔCAR+zipIL-2R T cells did not kill B7-H3-positive cell lines. B7-H3-negative cell lines were not killed by CAR T cells with or without zipIL-2R, demonstrating that zipIL-2R expression does not alter the antigen specificity of B7-H3-CAR T cells.

Having established that expression of zipIL-2R does not change the specificity of CAR T cells, a standard repeat stimulation assay with tumor cells to mimic chronic antigen exposure was performed. B7-H3 positive (FIGS. 4F, 4G, 4I-4J) or negative cell lines (FIG. 4E and FIG. 4H) were co-cultured with CAR T cells at 2:1 E:T ratio. After seven days, T cells were collected and counted. Stimulation with tumor cells was repeated at the same E:T ratio as long as CAR T cells continued to kill tumor cells and expand. When co-cultured with B7-H3-negative cell lines, none of the T cell populations killed tumor cells or expanded. However, functional B7-H3-CAR T cells were able to kill B7-H3-expressing tumor cells and expand. Co-expression of zipIL-2R in B7-H3-CAR T cells greatly enhanced CAR T cell expansion after exposure to tumor cells and enabled them to kill tumor cells after multiple encounters, as compared to unmodified B7-H3-CAR T cells (FIG. 4G and FIG. 4J).

### Example 4. ZipIL-2R Improves B7-H3-CAR T Cell Antitumor Activity In Vivo

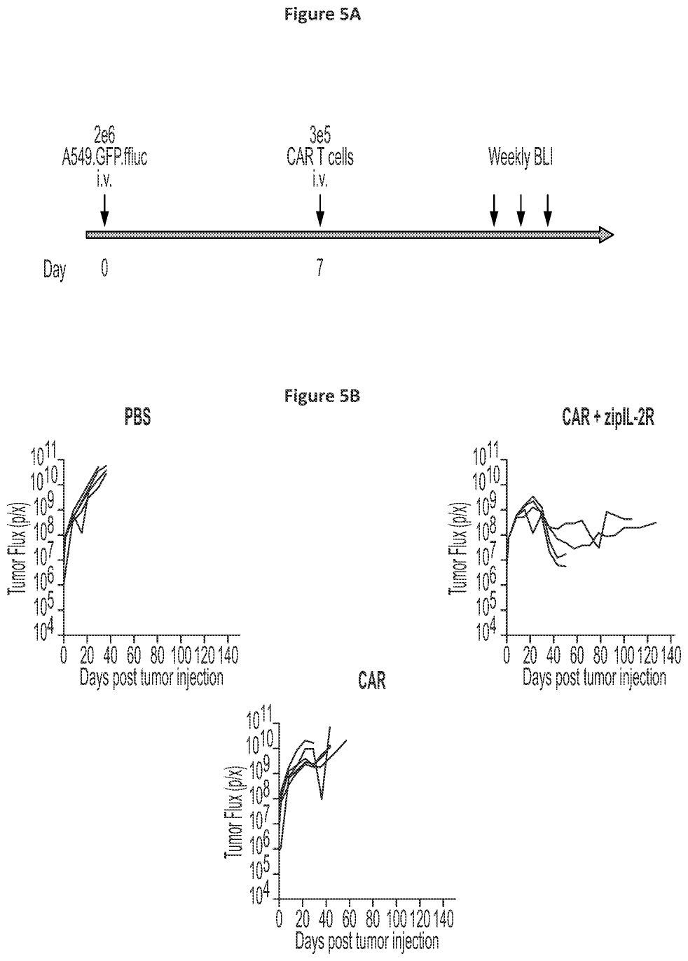

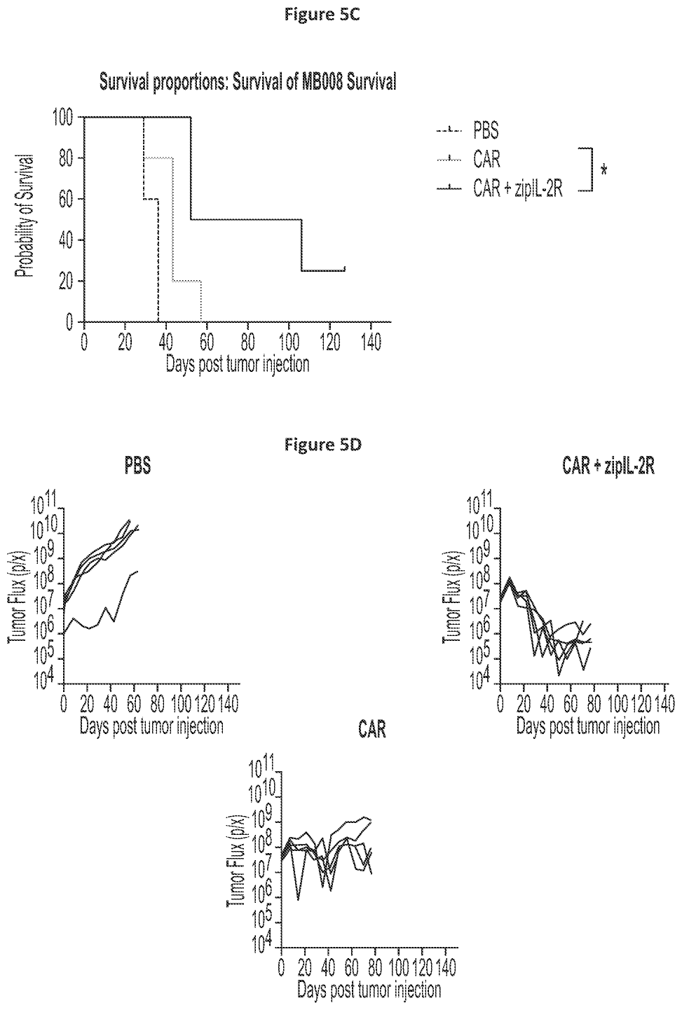

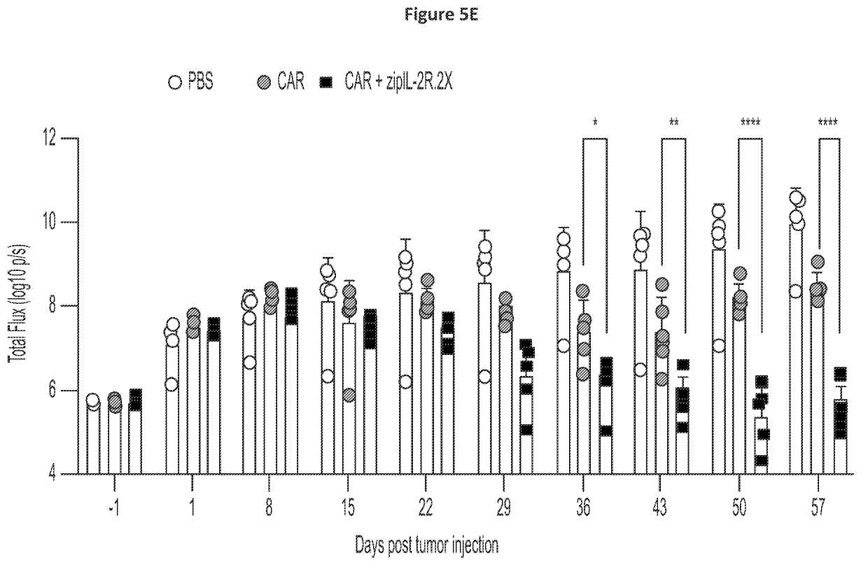

In order to compare the in vivo antitumor activity of B7-H3-CAR and B7-H3-CAR+zipIL-2R T cells, the present experiment used a A549 tumor model in which intravenous (i.v.) injection of firefly luciferase (ffluc)-expressing A549 cells results in tumor formation in the lungs, may be quantified by bioluminescence imaging (BLI) (FIG. 5A). Two independent experiments were performed with two donors: donor 1 (FIG. 5B-5C) and donor 2 (FIG. 5D-5E), and for both donors, B7-H3-CAR+zipIL-2R T cells had significantly improved antitumor activity. In these experiments, low doses of T cells (3×10⁵ per mouse) at which unmodified B7-H3-CAR T cells had limited activity were used.

In summary, the data provided in FIGS. 2-4 highlight that the generated zipIL-2R was functional, and that expression of zipTL-2R in CAR T cells improved their effector function, including antigen-dependent antitumor activity, in vitro as well as in vivo.

### Example 5. 2X zipReceptor Design can be Re-Engineered to Provide IL-7 and IL-21 Signaling to CAR T Cells to Improve Antitumor Activity In Vitro

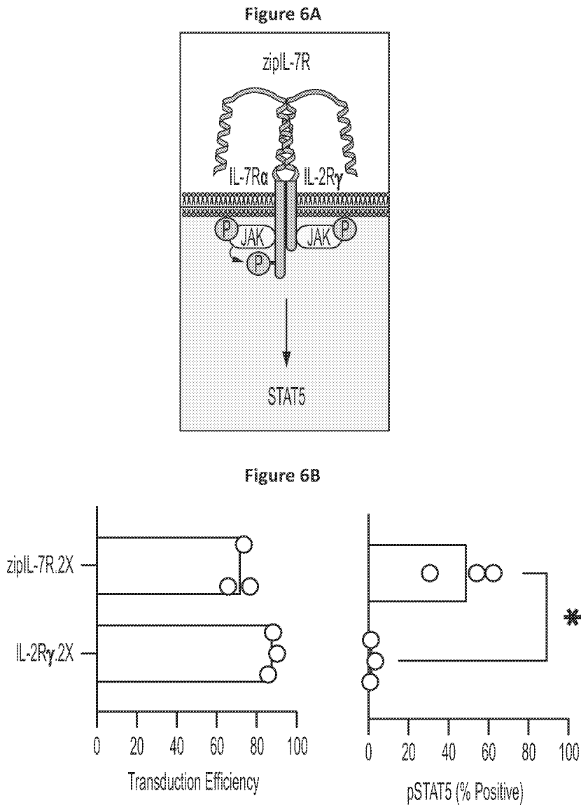

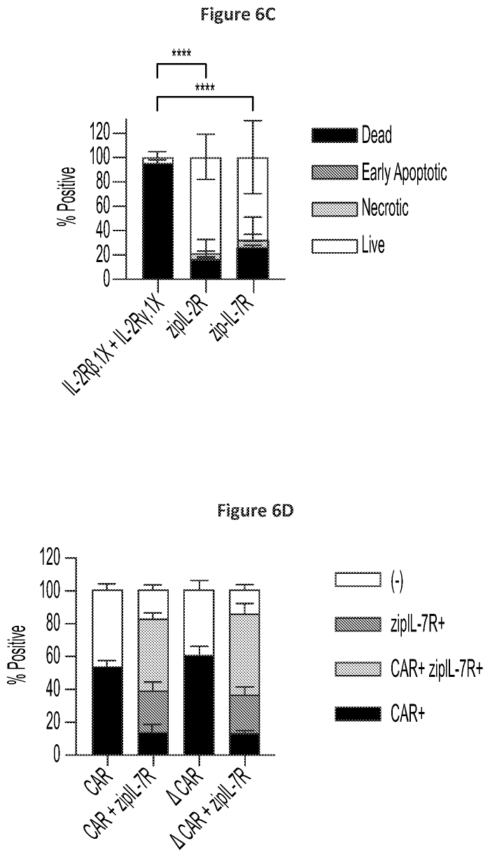

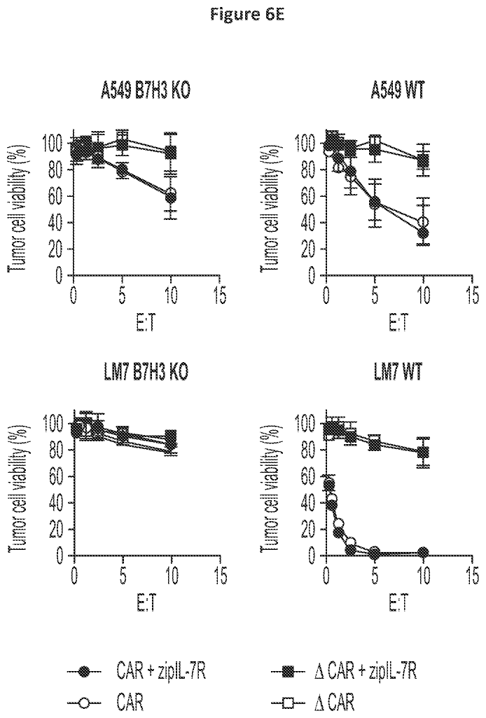

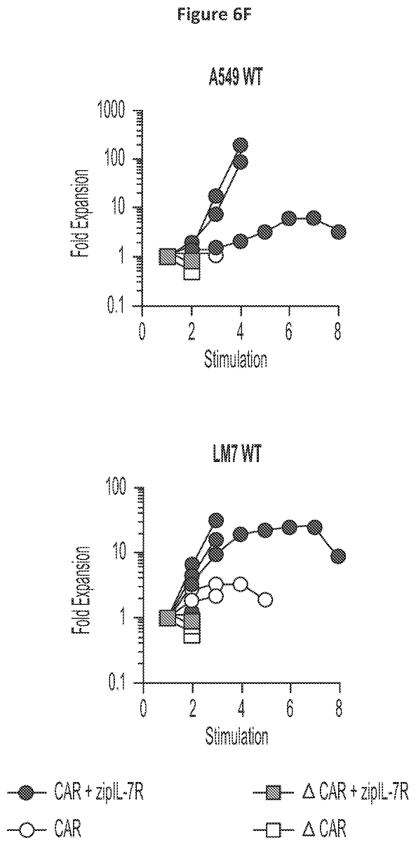

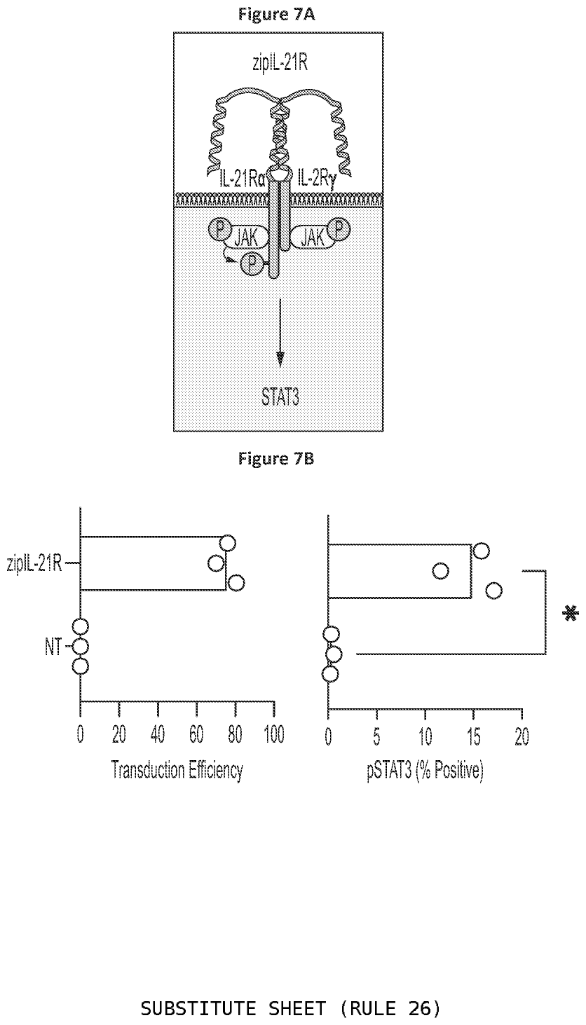

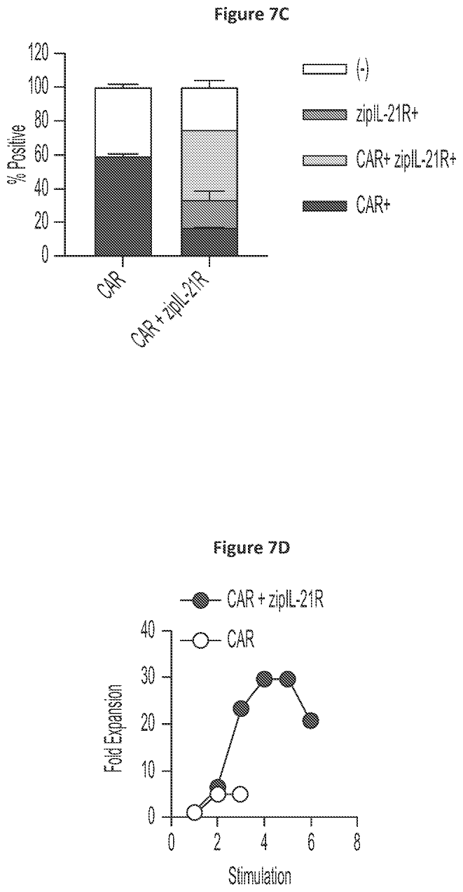

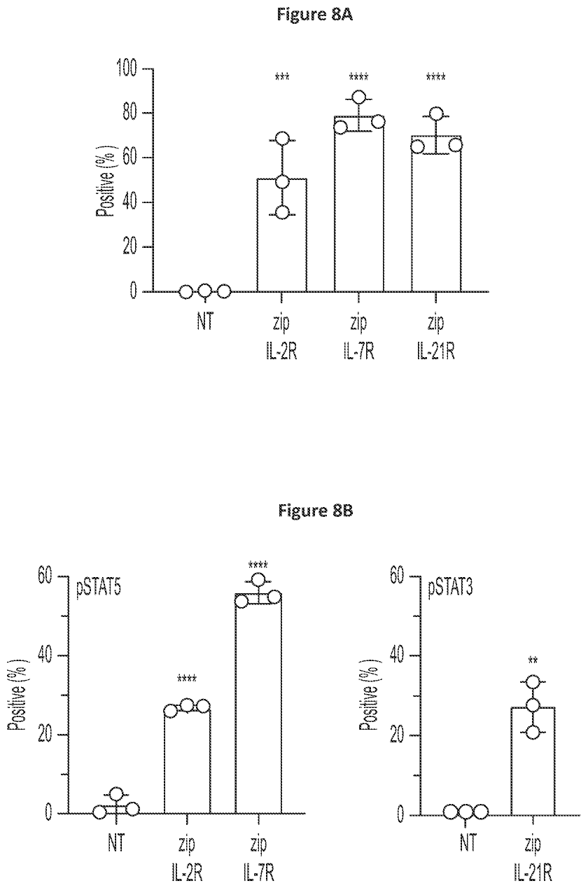

The generated zipIL-2R activated STAT5, PI3K, and ERK pathways. However, since the zipReceptor system has modular components, it allows for immune cell reprogramming to tailor activation of different intracellular signaling pathways. To demonstrate feasibility, an IL-7 zipReceptor (zipIL-7R) (FIG. 6A-6F), which activates STAT5 and PI3K, and an IL-21 zipReceptor (zipIL-21R) (FIG. 7A-7D), which activates STAT3, PI3K, and ERK, were generated for subsequent evaluation of their respective functionality. Following transduction of human T cells, zipIL-7R was robustly expressed and activated STAT5 at baseline (FIG. 6B). zipIL-7R improved survival in a cytokine starvation assay, similar to zipIL-2R (FIG. 6C). In contrast, T cells that were transduced with both chains of the 1X zipIL-2R had all died, further confirming the need for two pairs of leucine zipper motifs to generate active zipReceptors.

To test whether zipIL-7R improves B7-H3-CAR T cell antitumor activity in vitro, human T cells were transduced with a functional B7-H3-CAR (CAR) or non-functional B7-H3-CAR (ΔCAR) or co-transduced with a CAR and zipIL-7R (CAR+zipIL-7R, ΔCAR+zipIL-7R) (FIG. 6D). CAR and CAR+zipIL-7R T cells killed B7-H3-expressing tumor cells in a 24-hour MTS assay but were unable to kill B7-H3 KO tumor cells, demonstrating that zipIL-7R did not alter the antigen specificity of B7-H3-CAR T cells (FIG. 6E). ΔCAR T cells with or without zipIL-7R did not kill B7-H3-positive or knock-out (KO) cells, demonstrating that the observed tumor cell killing depends on the expression of a functional CAR. Next, the sequential killing activity of B7-H3-CAR T cells expressing zipIL-7R was interrogated. zipIL-7R expression improved the ability of B7-H3-CAR T cells to sequentially kill B7-H3-expressing tumor cells for all evaluated T cell donors (FIG. 6F).

Finally, to demonstrate that the generated zipIL-21R was functional, baseline activation of STAT3 in transduced human T cells was evaluated (FIG. 7B). B7-H3-CAR+zipIL-21R T cells (FIG. 6C) were generated as described above for the other zipReceptors, and these cells had an improved ability to sequentially kill tumor cells in a standard repeat stimulation assay in comparison to unmodified B7-H3-CAR T cells (FIG. 7D).

### Example 6. 2X zipReceptor Expression in Natural Killer (NK) Cells Activates STAT5/3 Signaling and Improves Survival

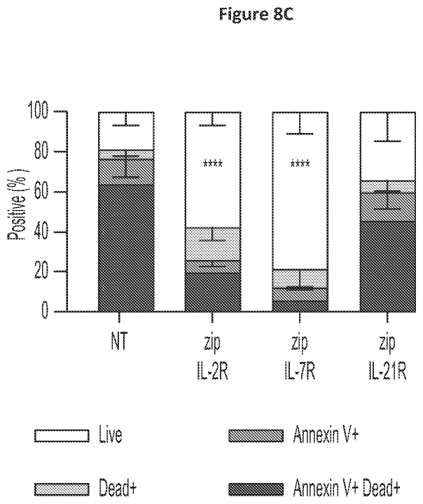

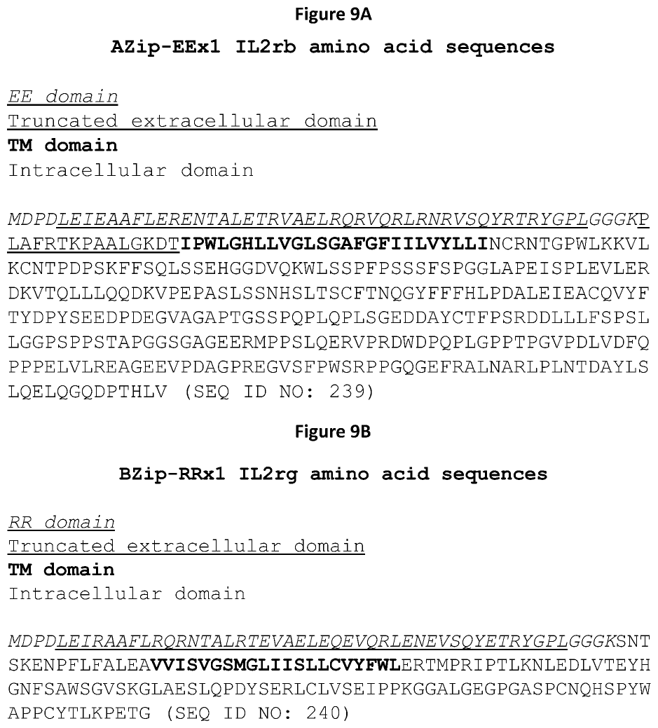

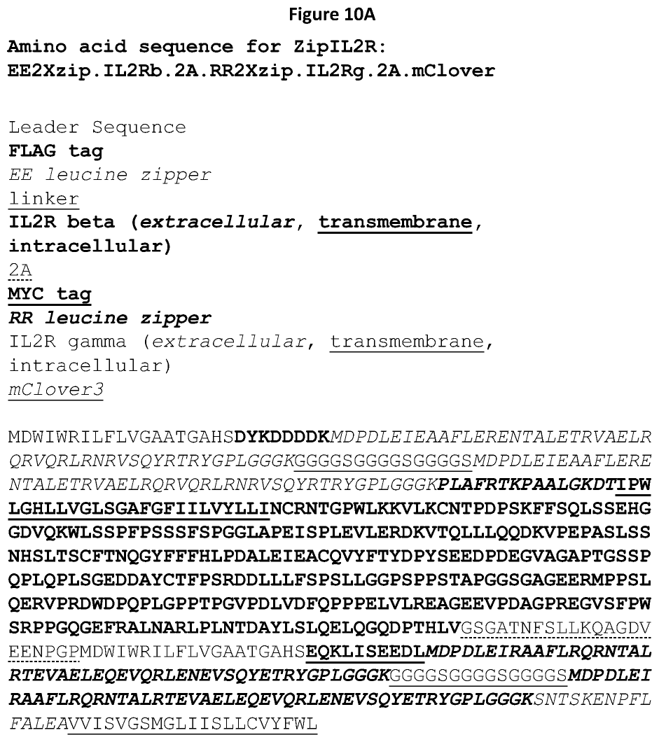

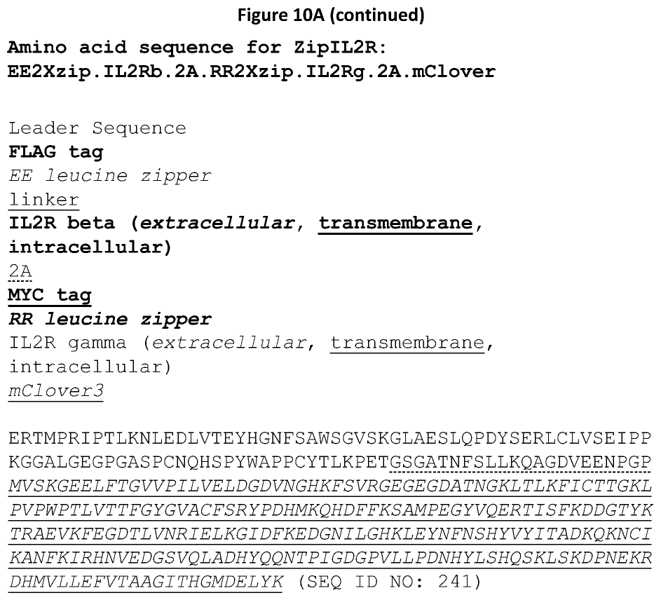

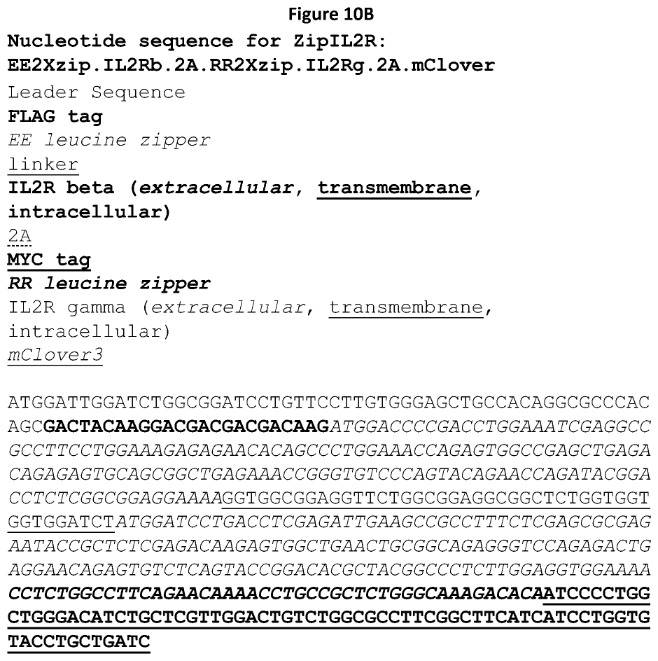

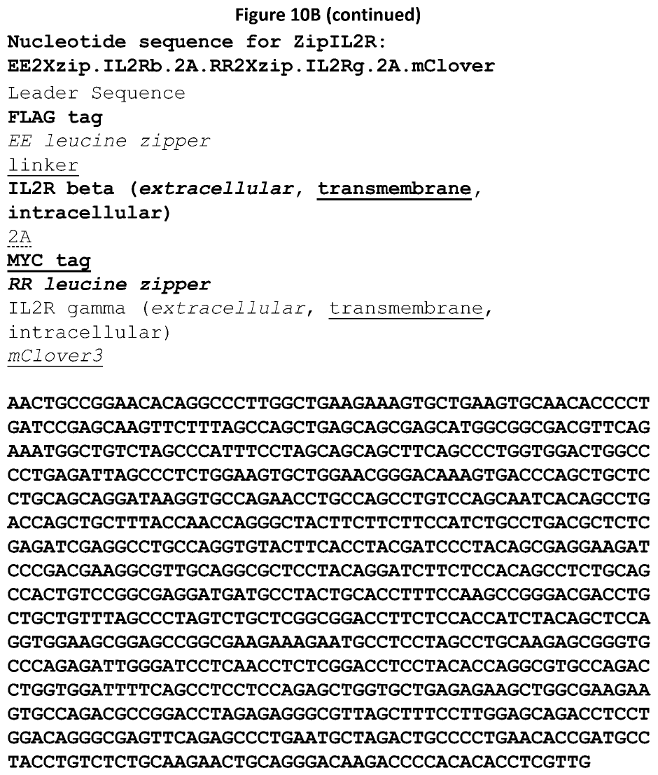

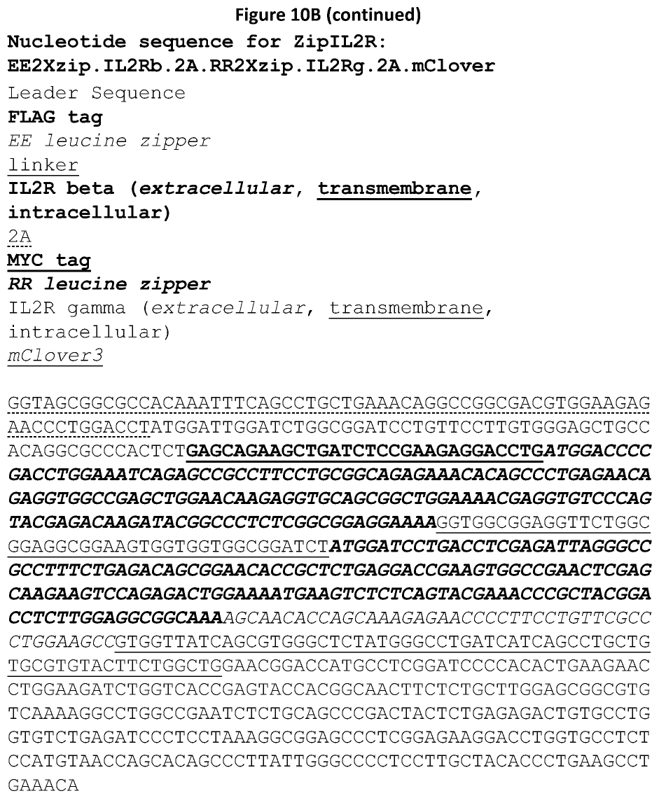

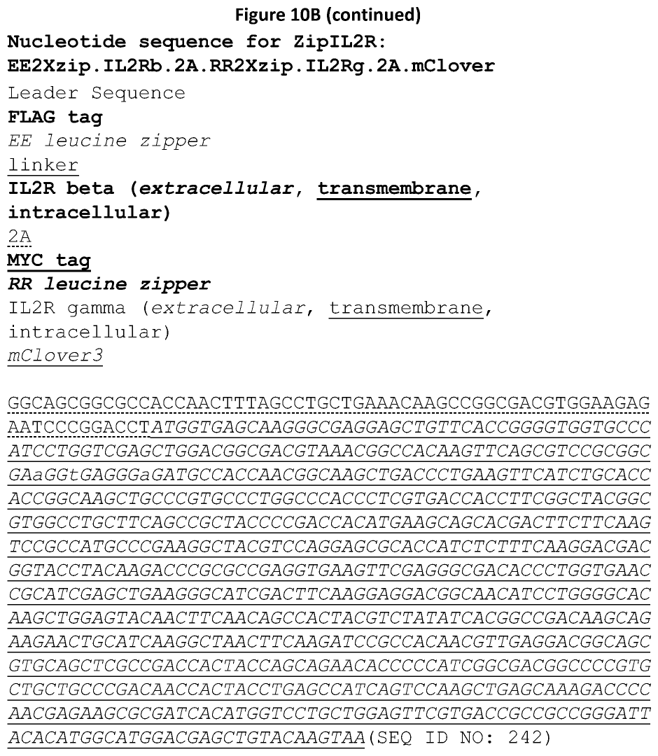

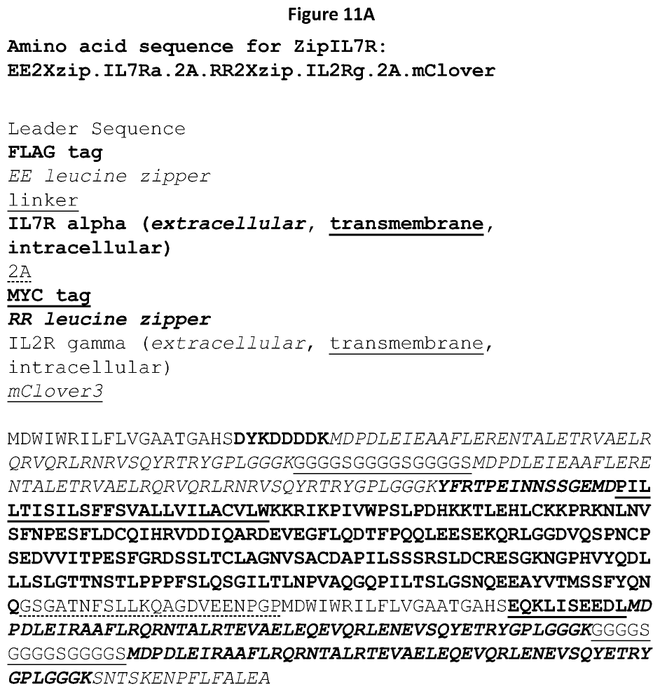

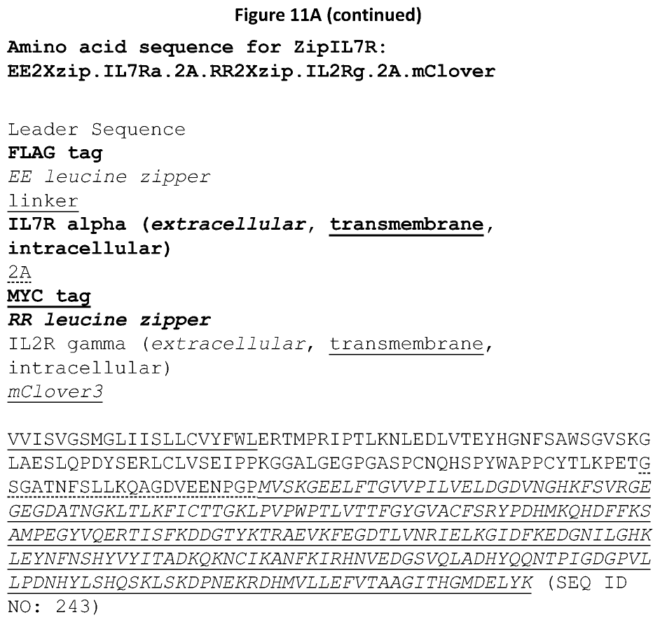

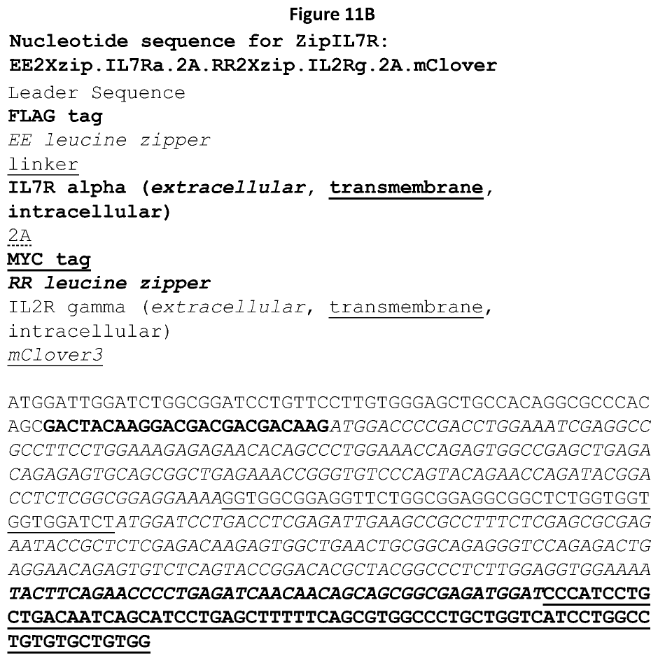

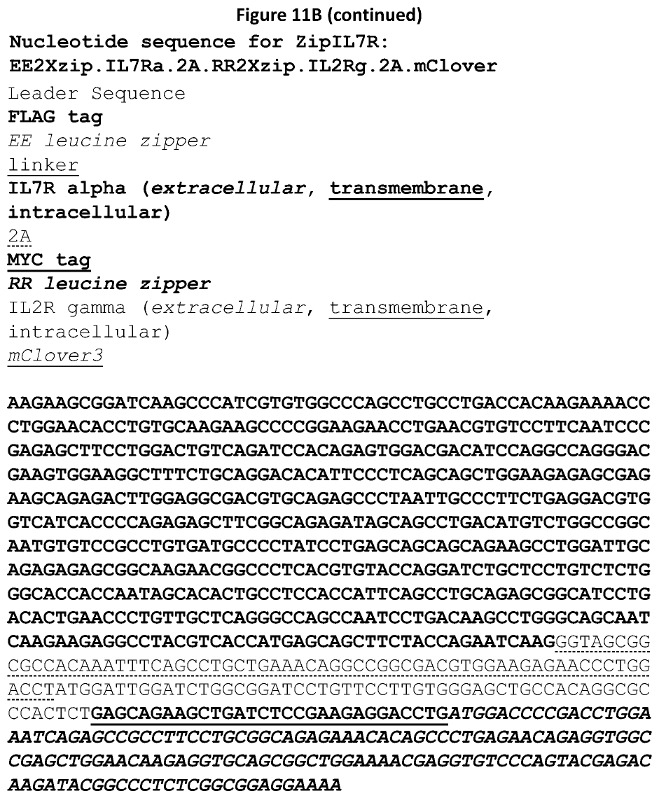

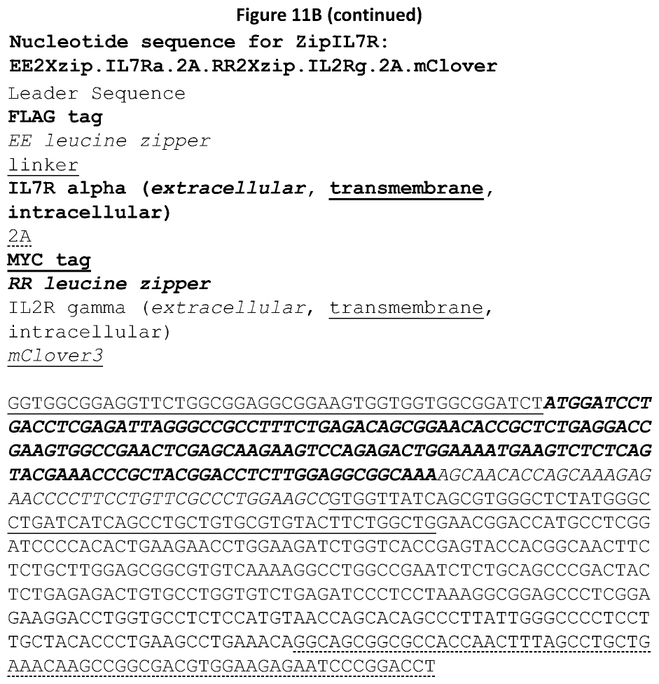

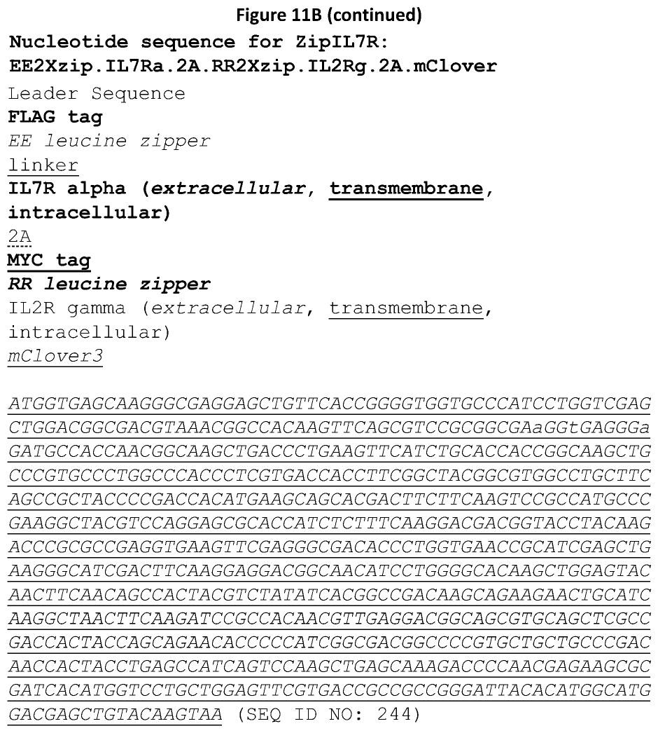

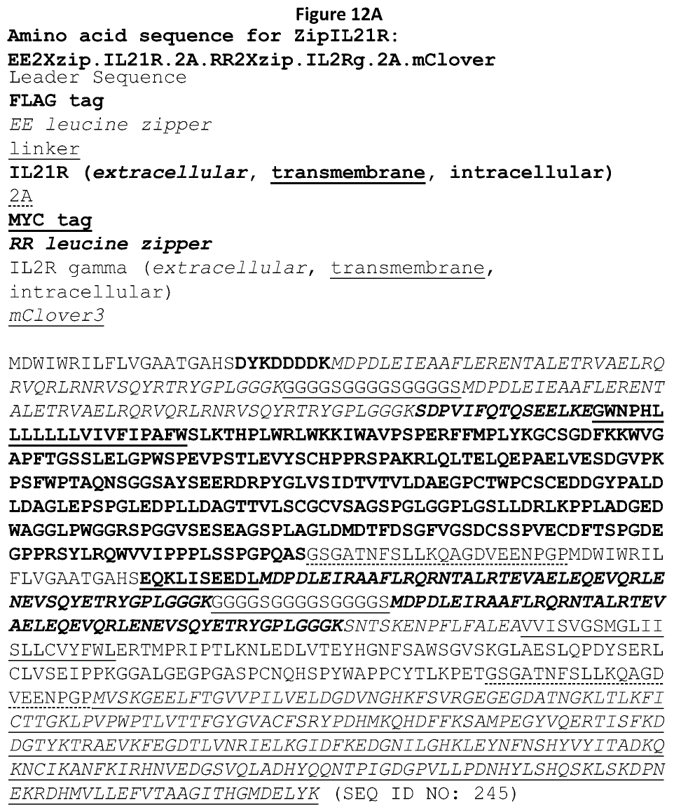

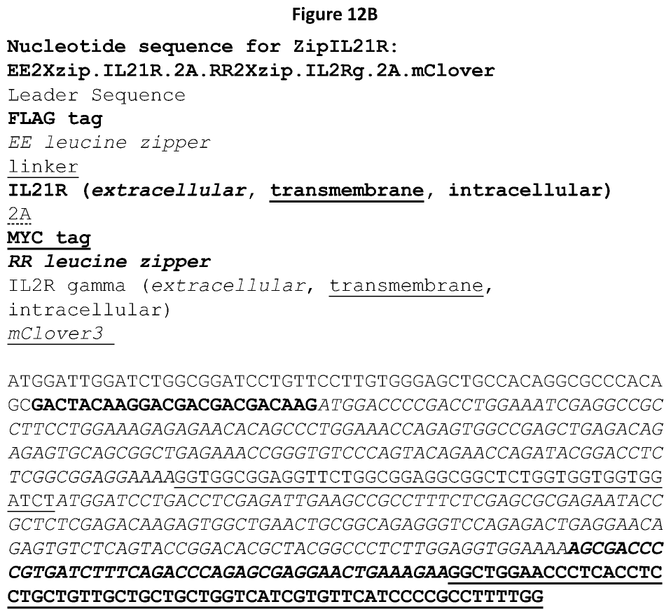

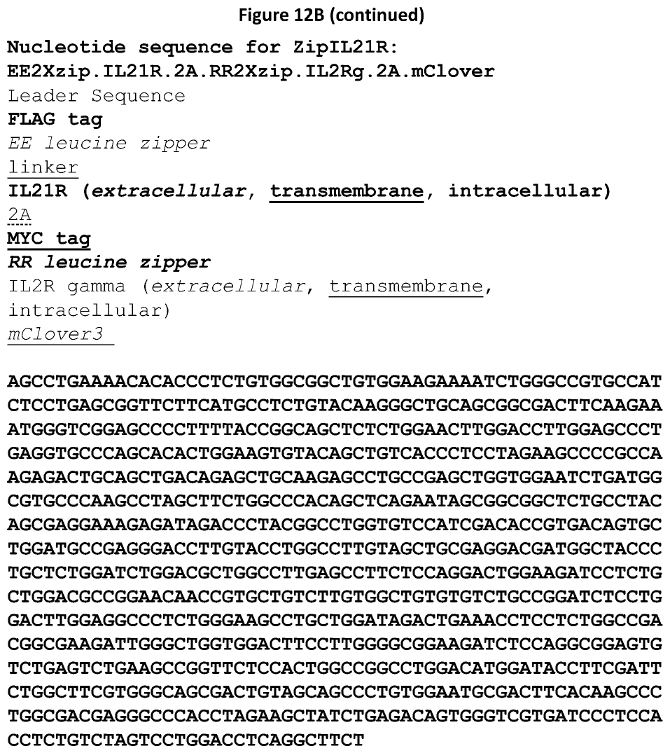

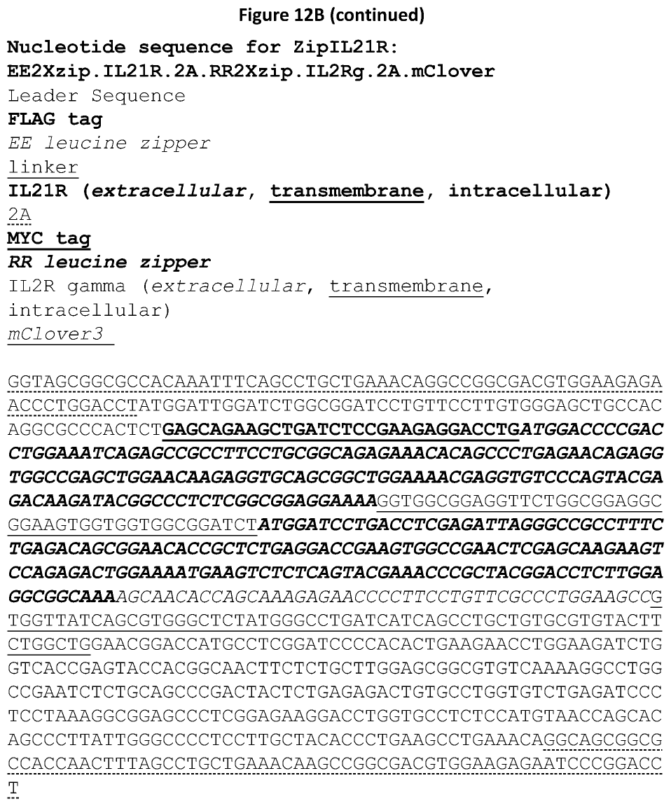

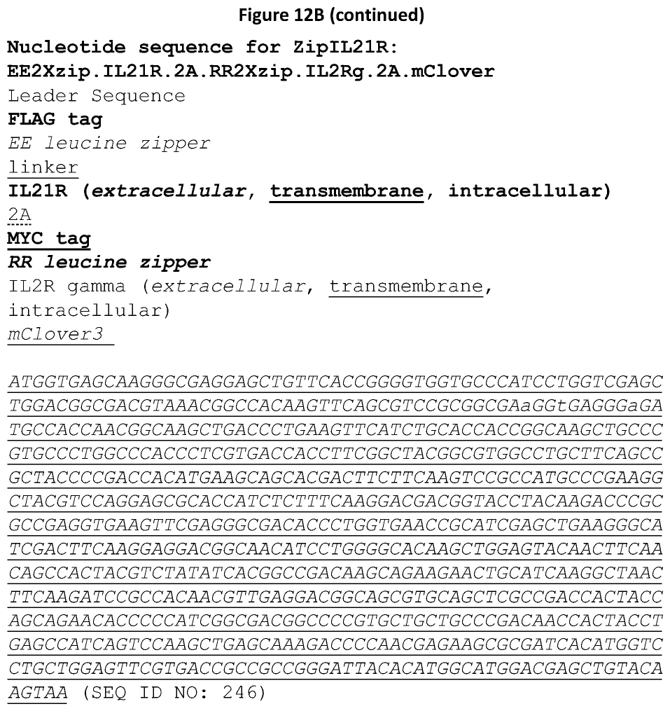

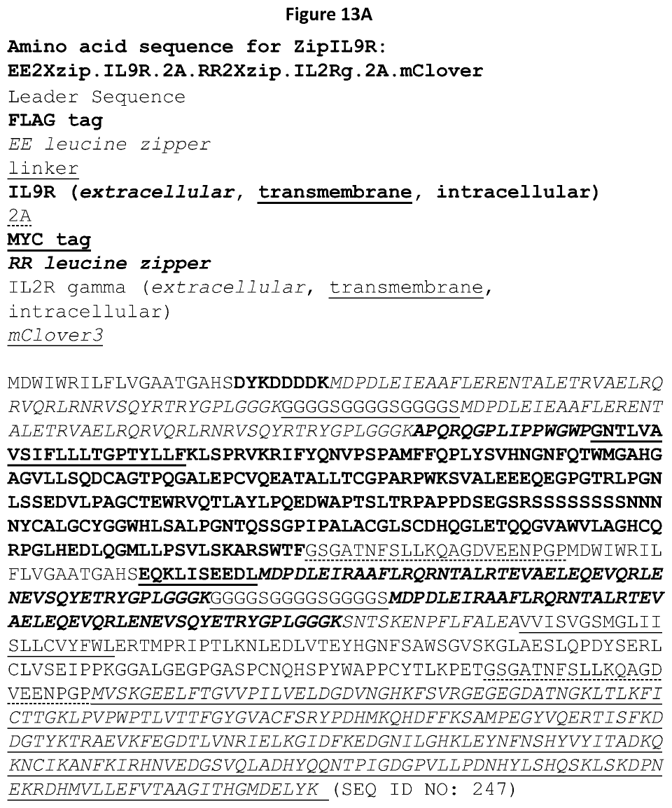

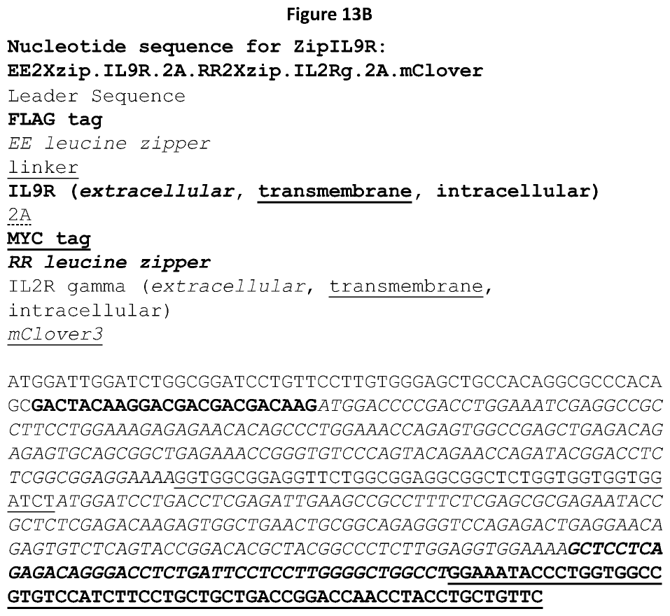

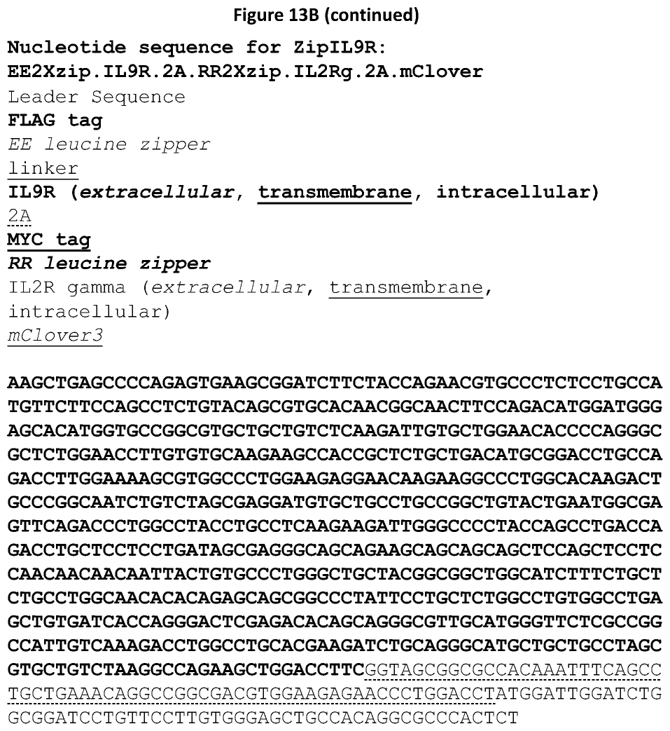

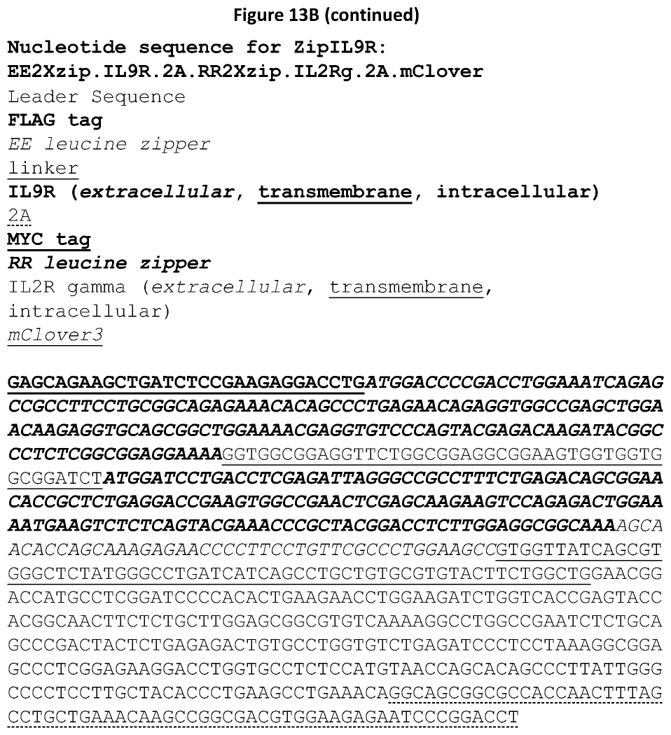

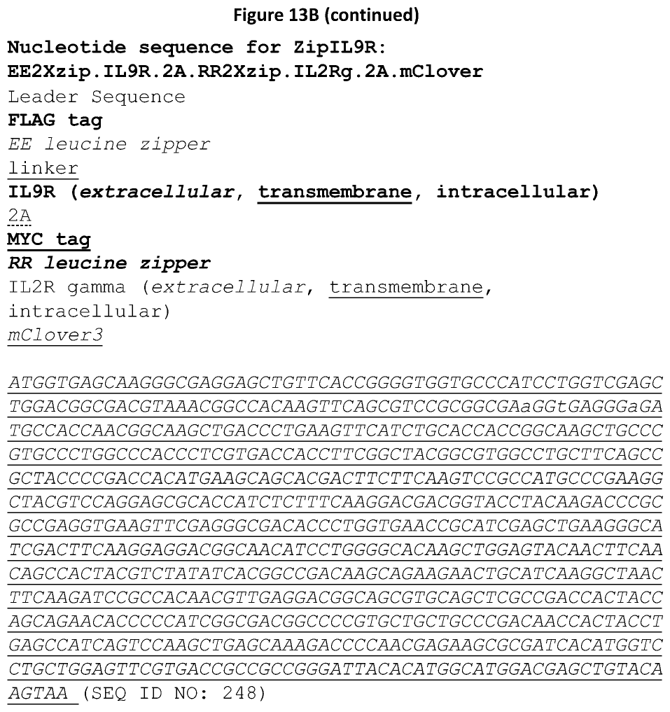

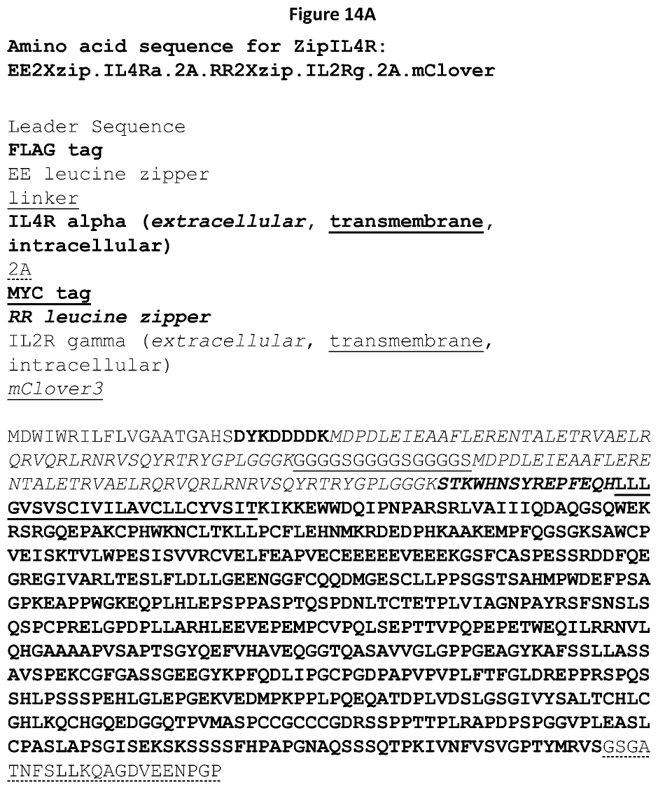

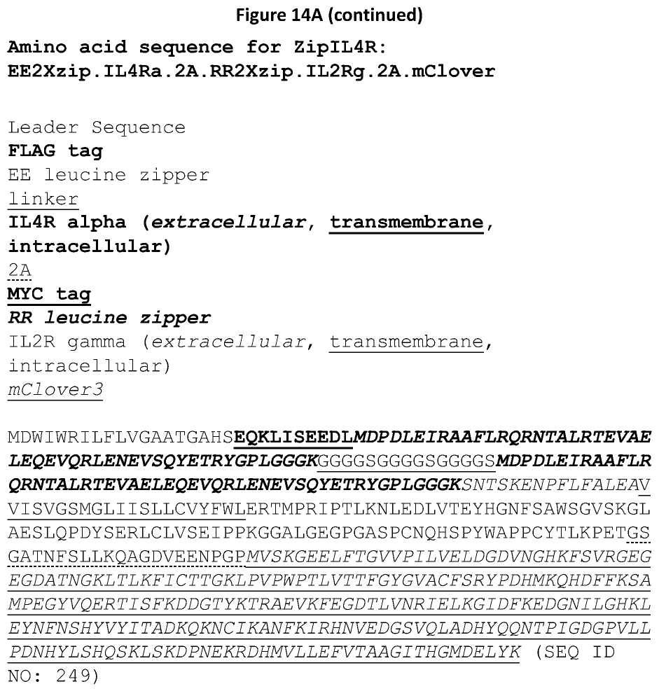

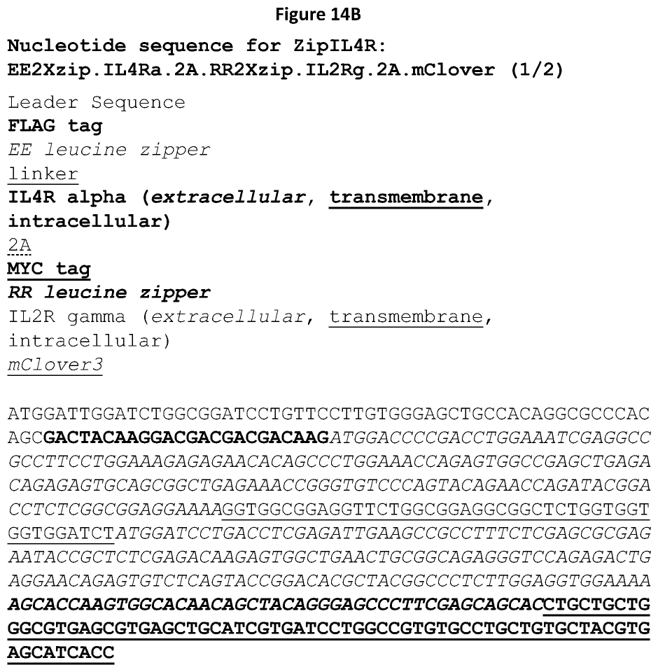

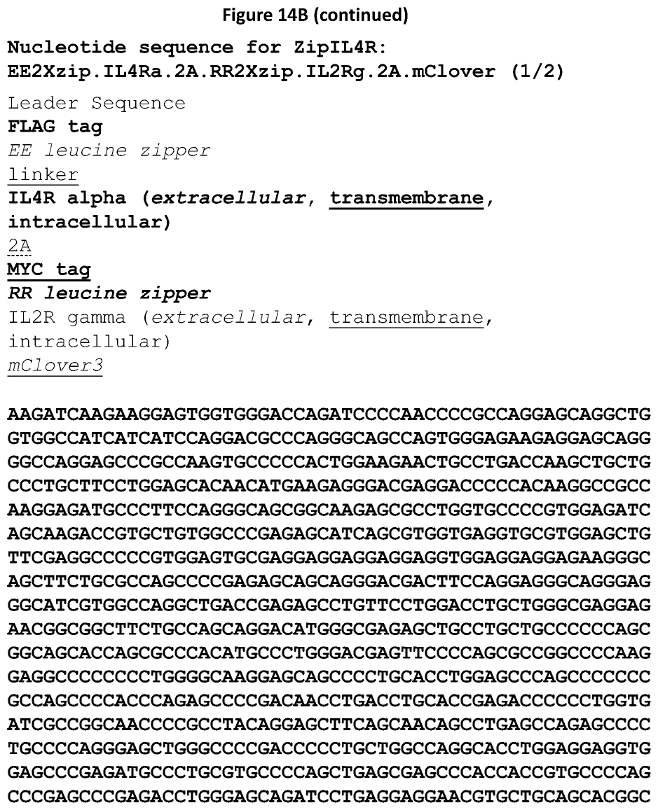

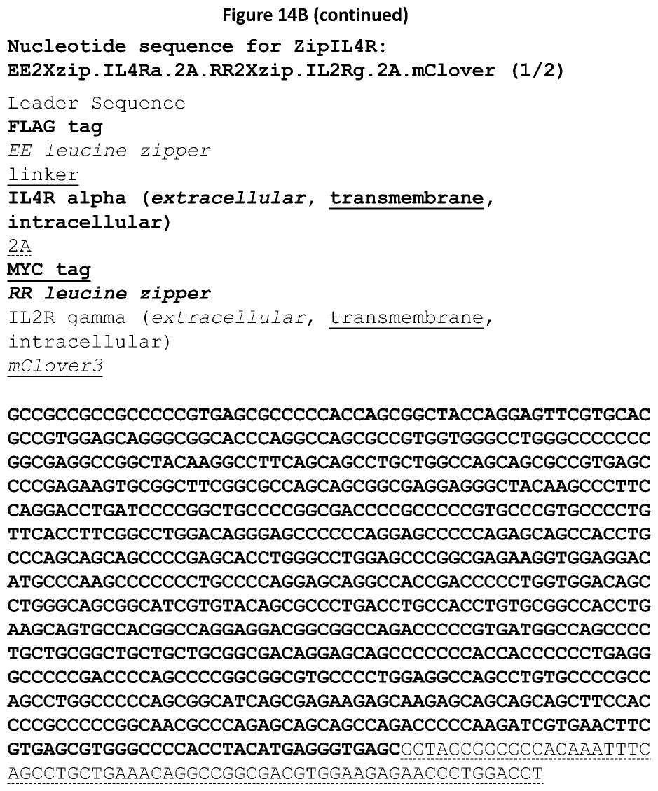

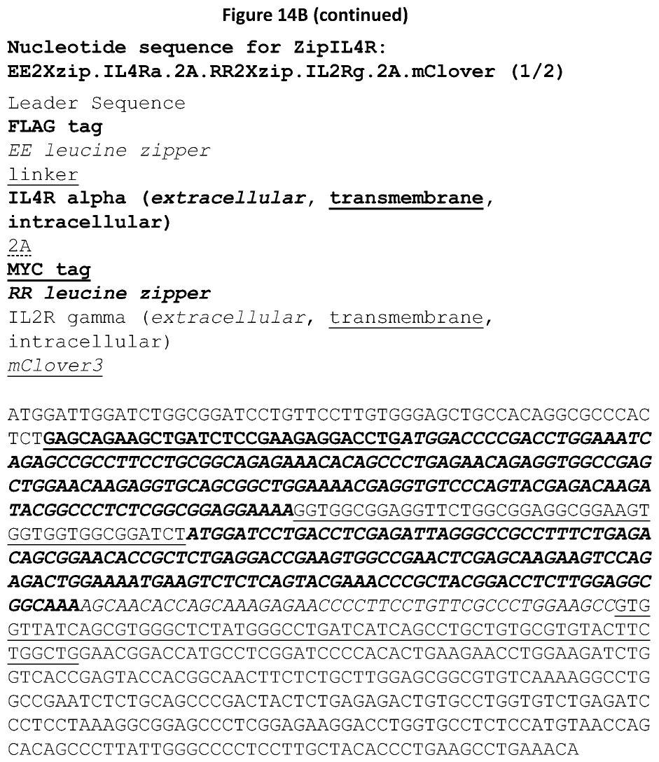

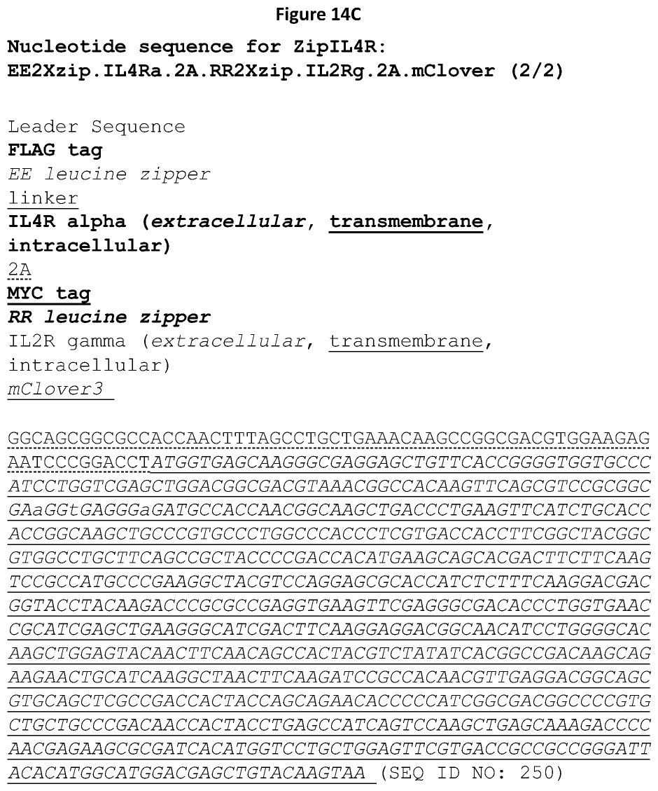

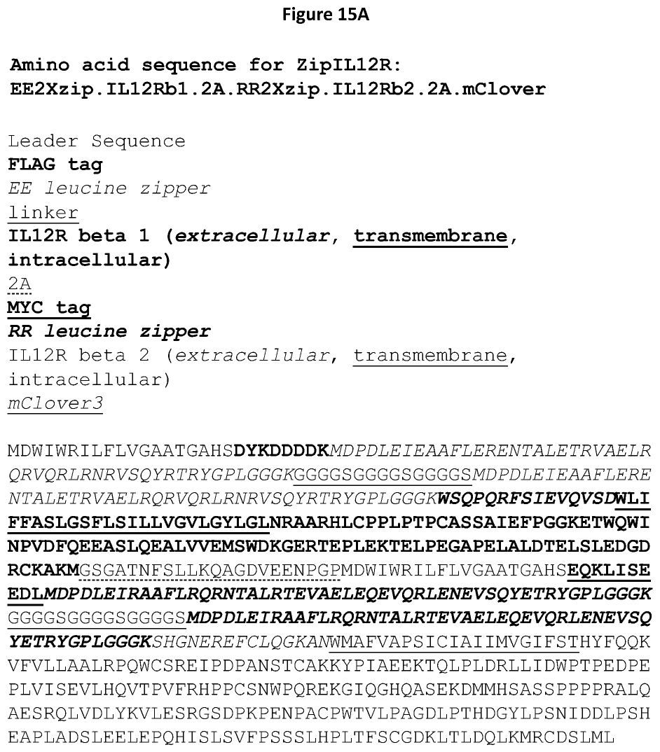

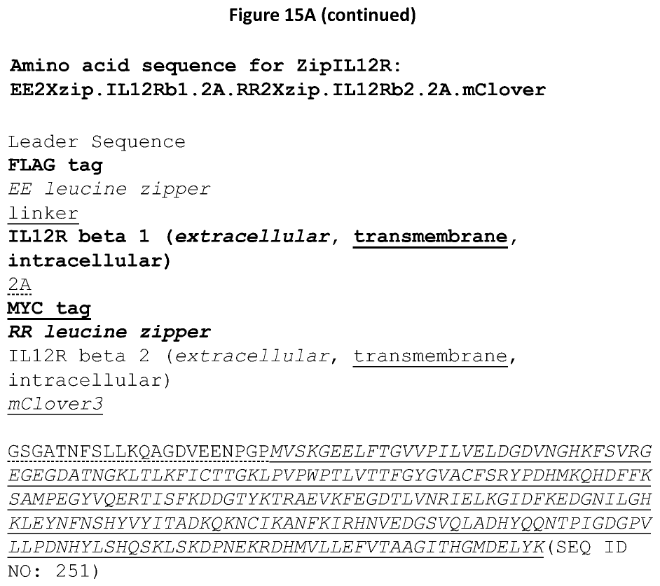

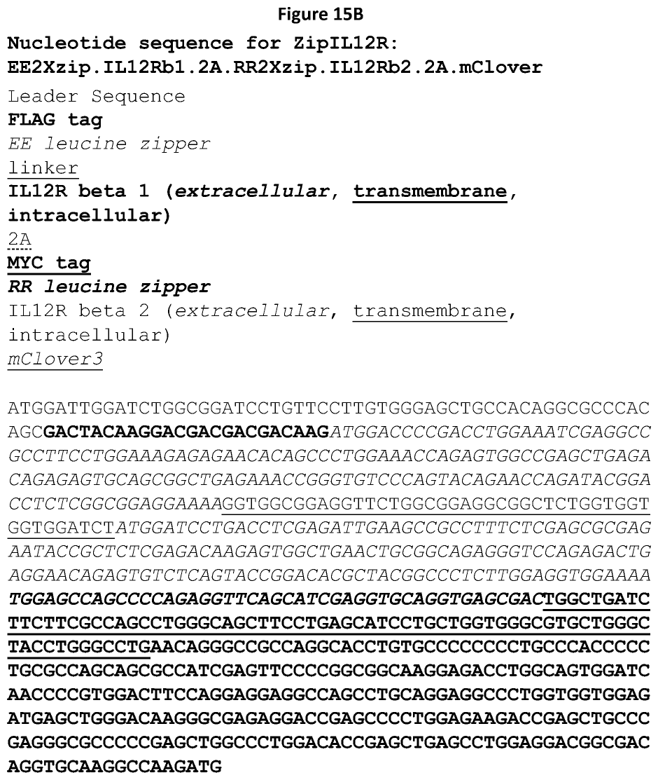

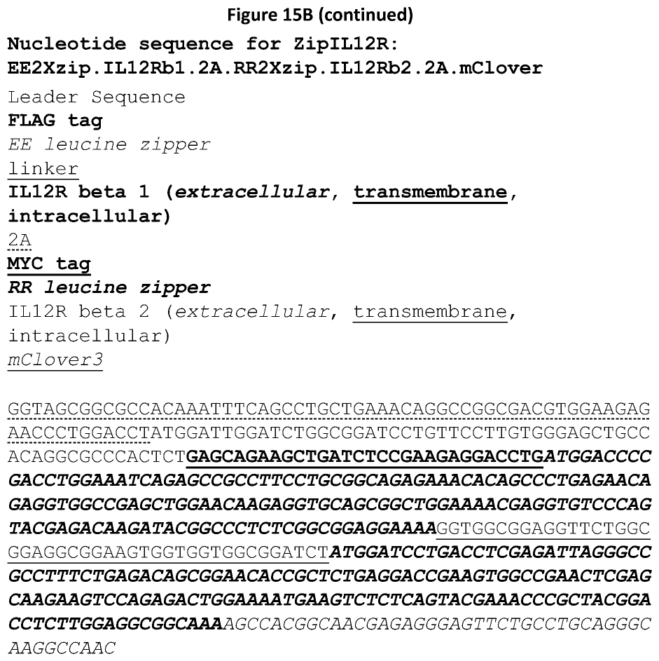

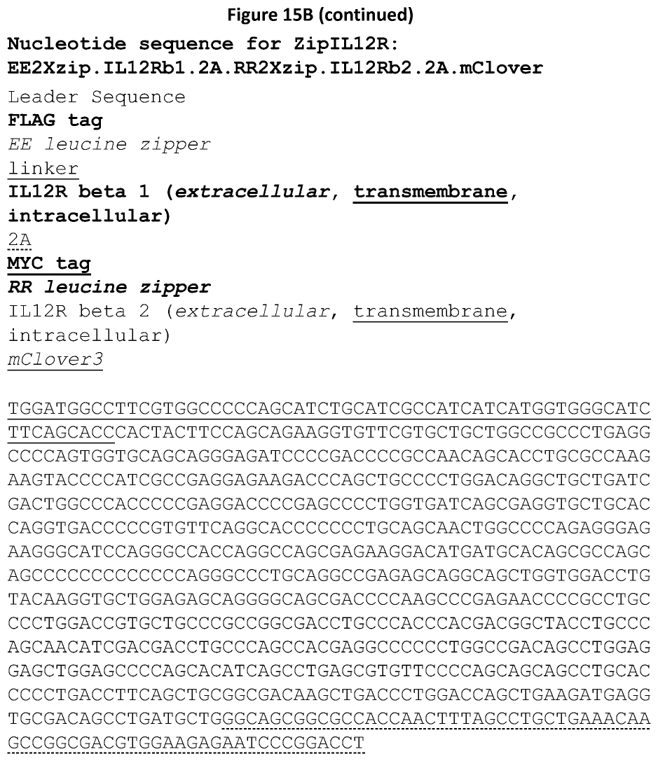

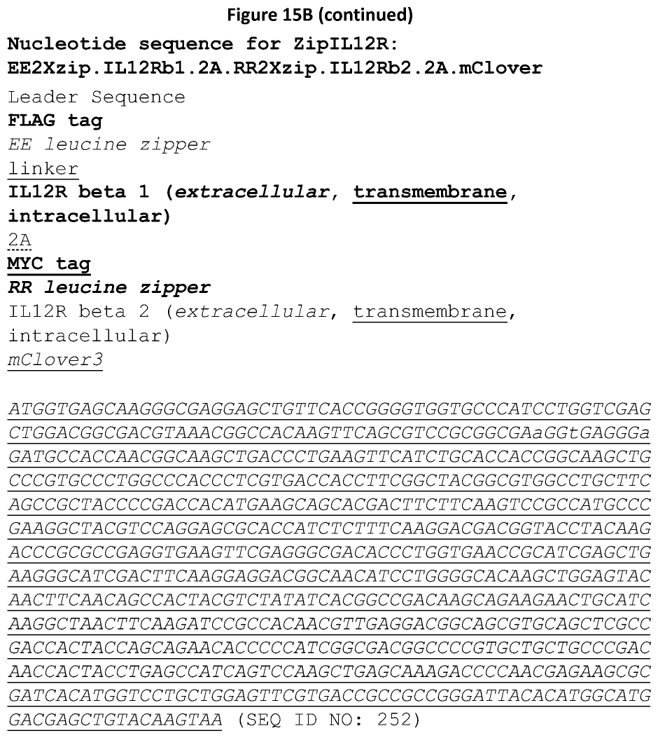

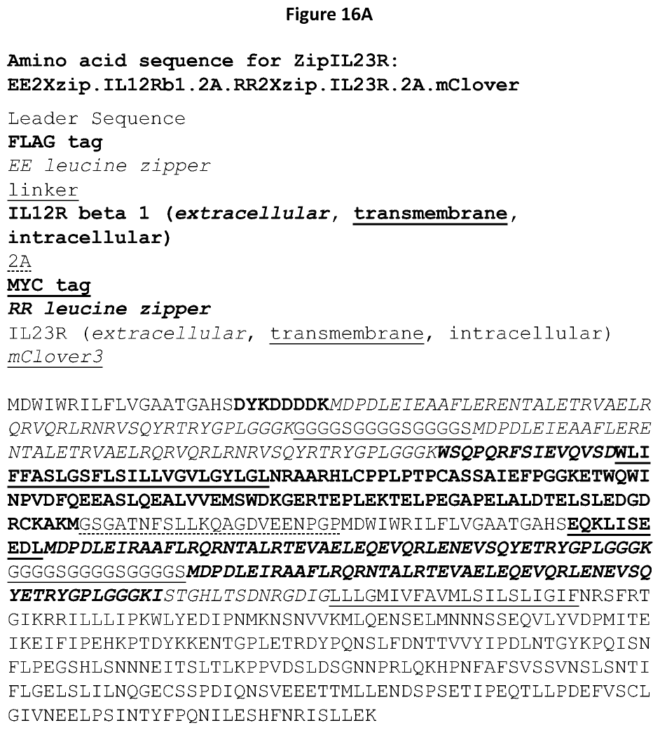

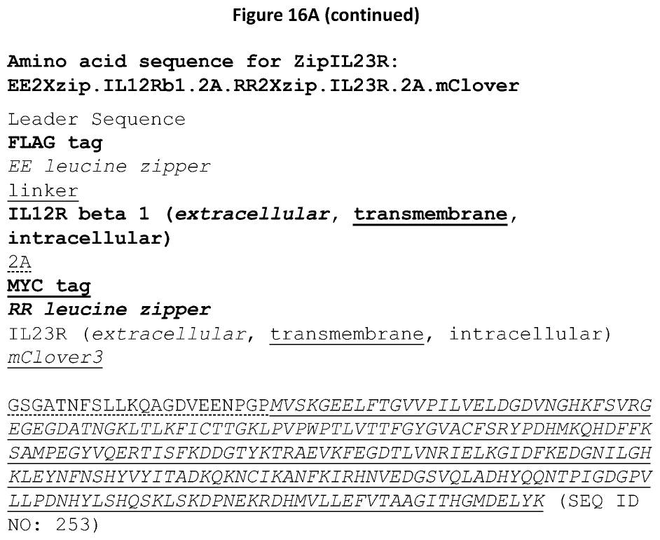

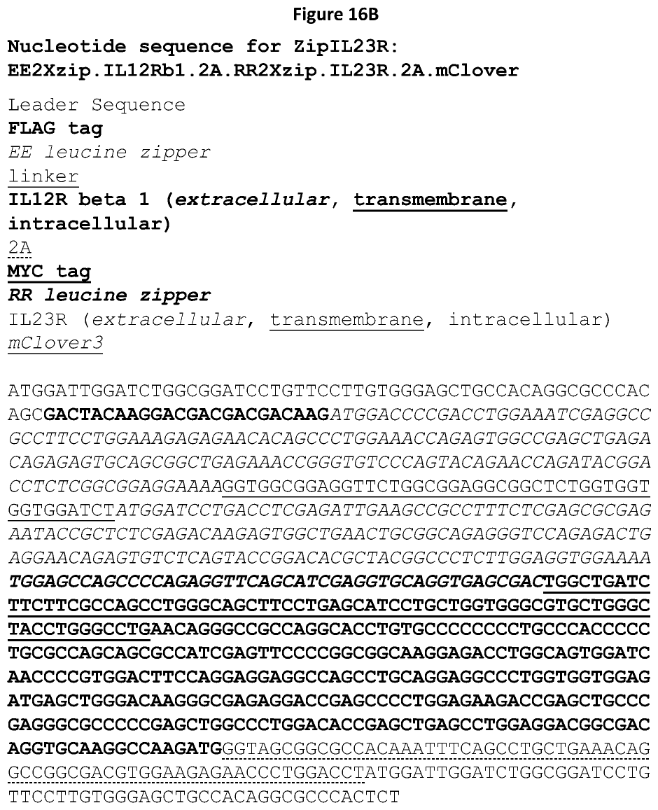

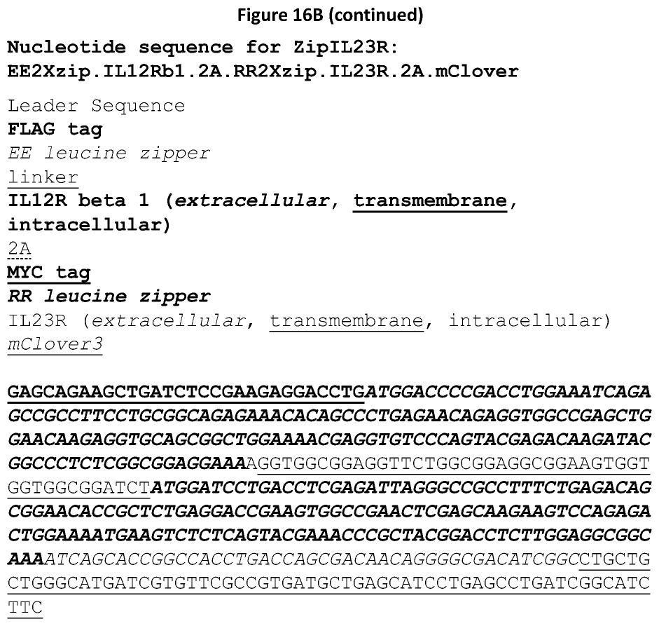

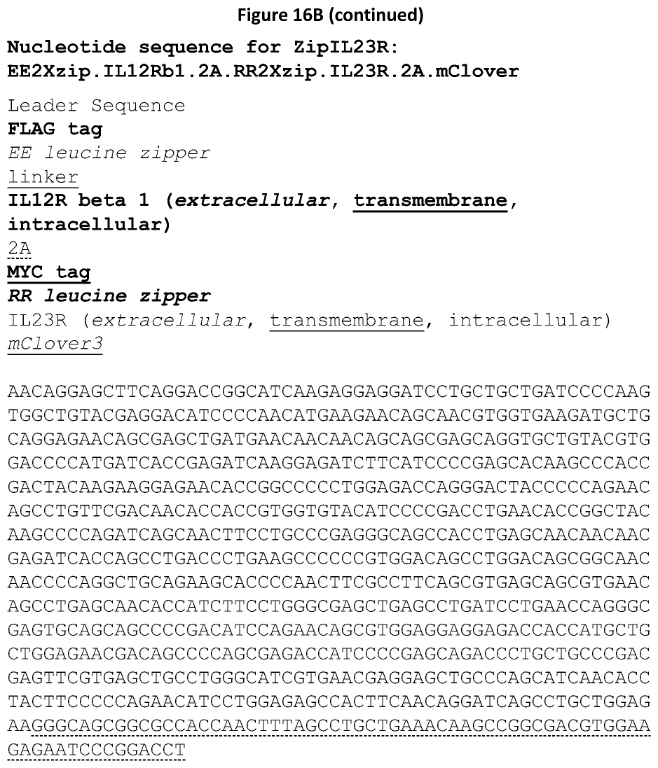

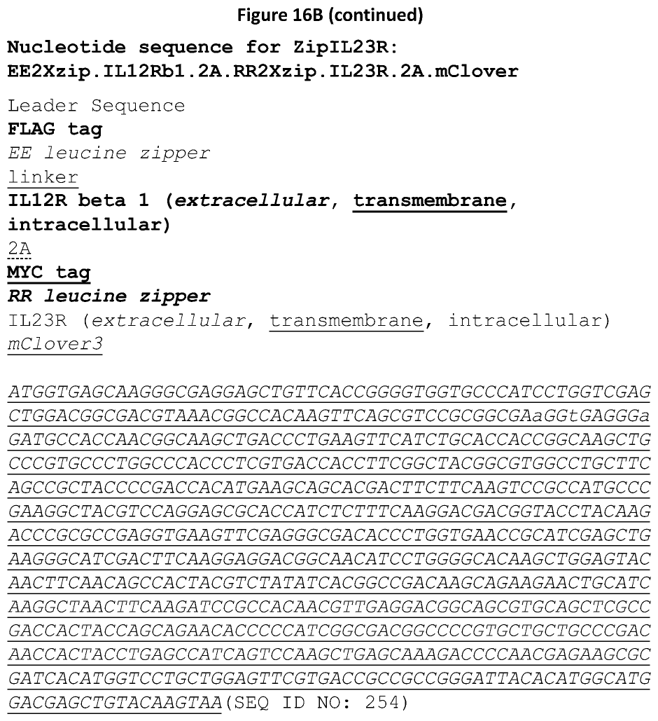

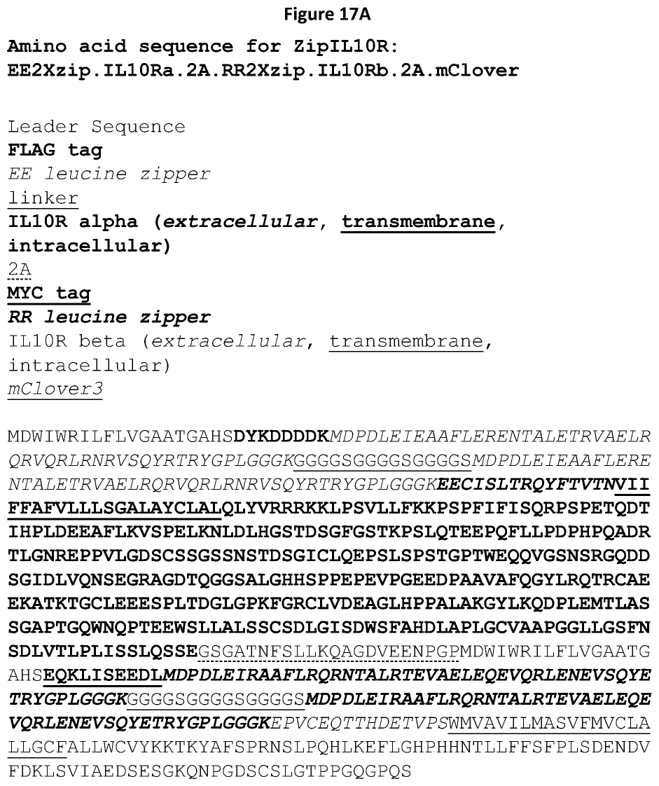

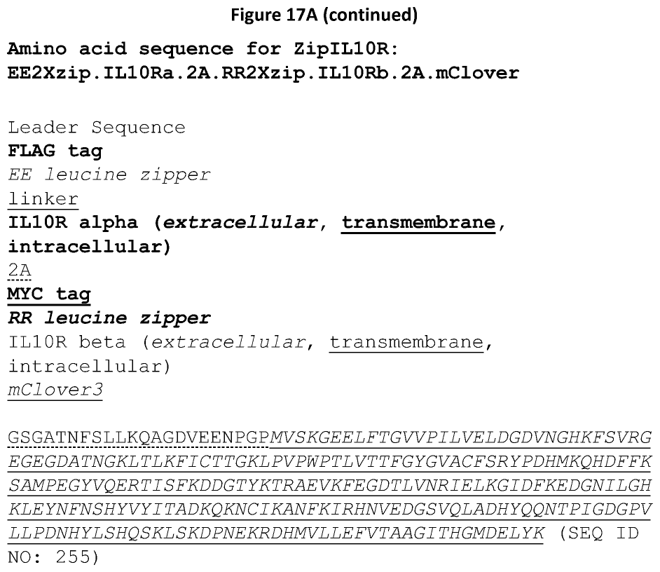

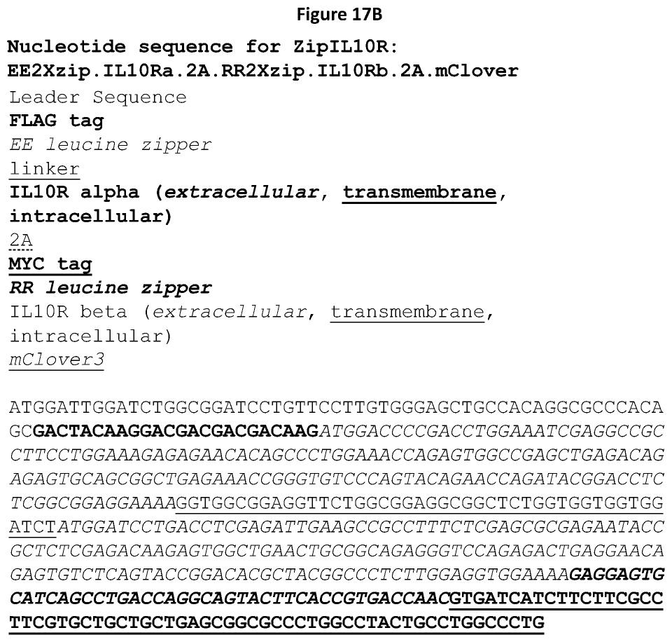

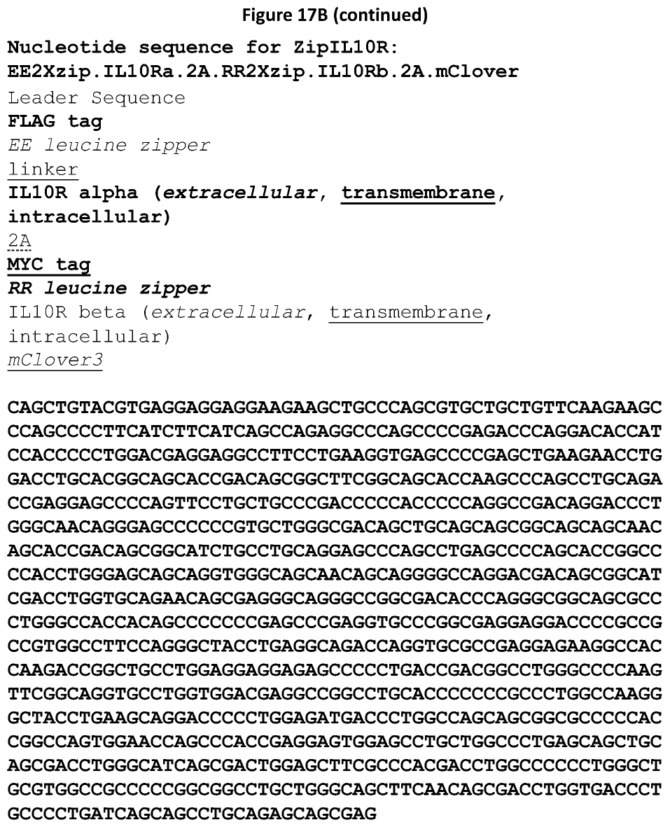

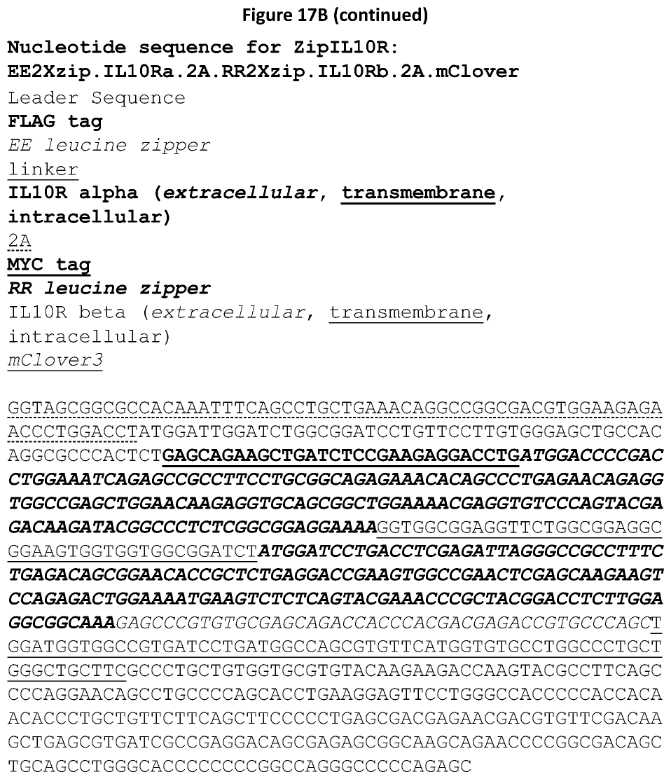

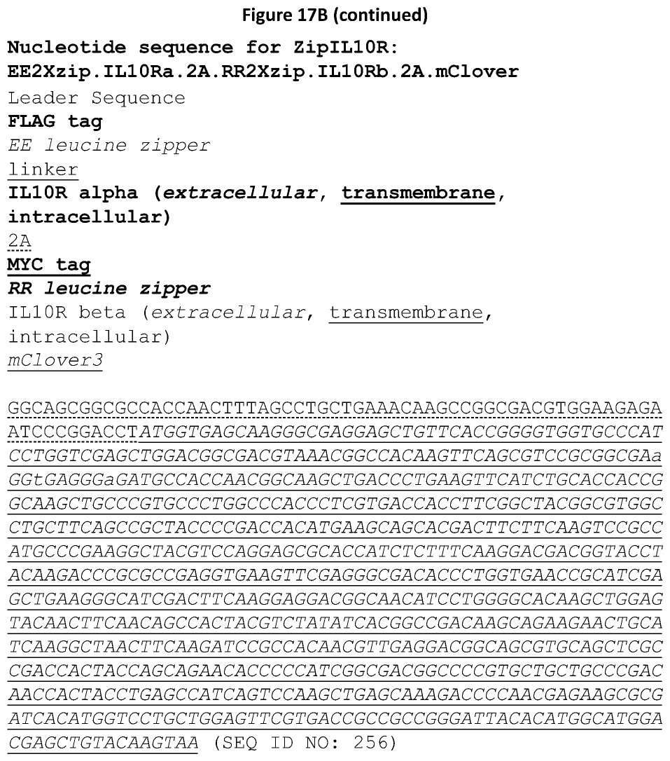

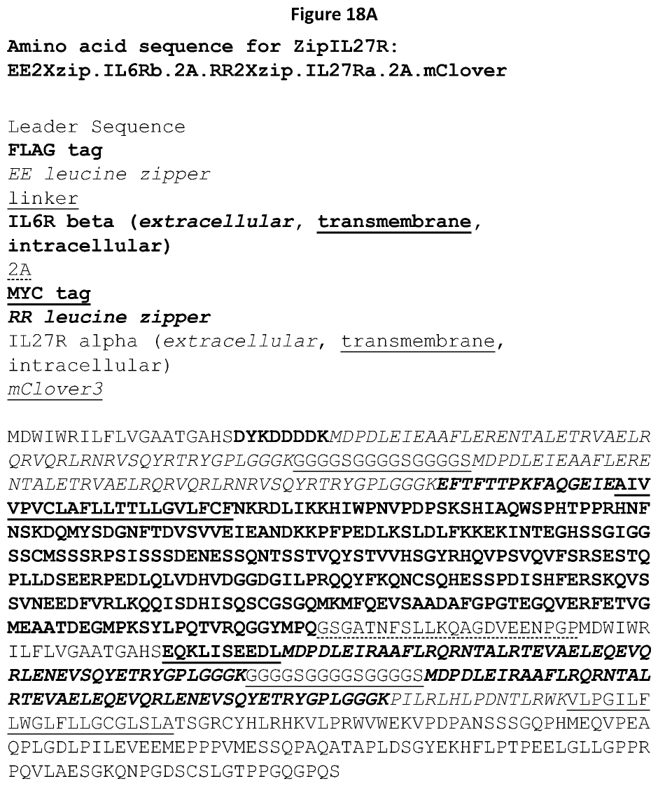

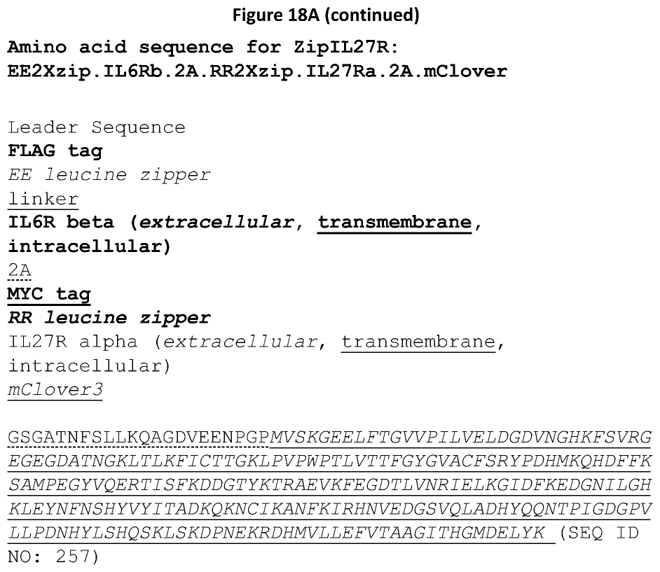

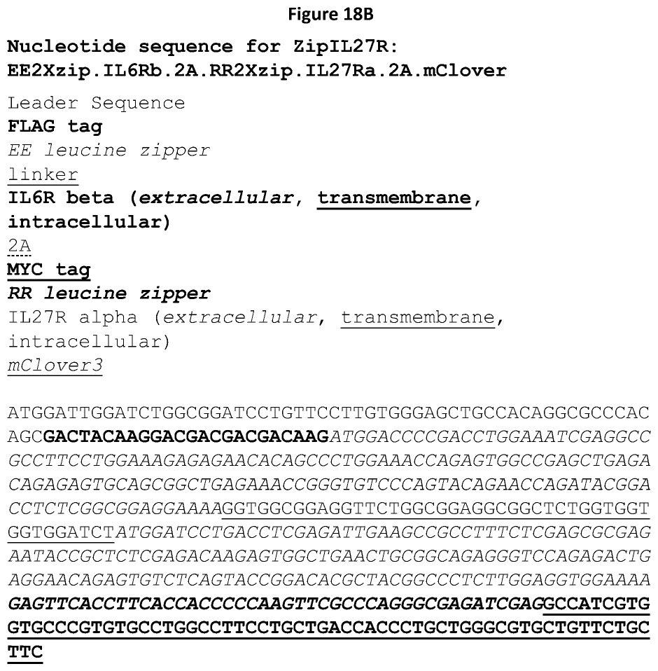

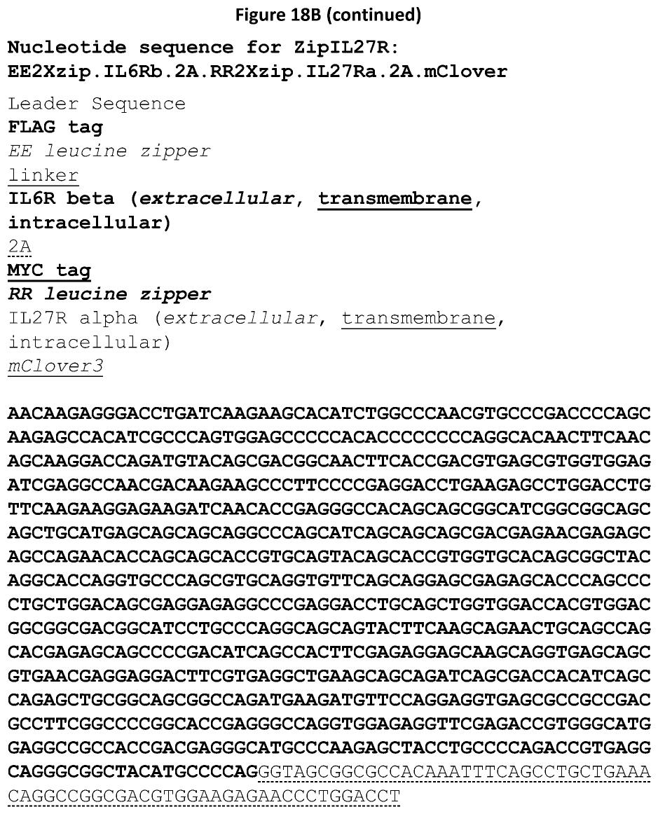

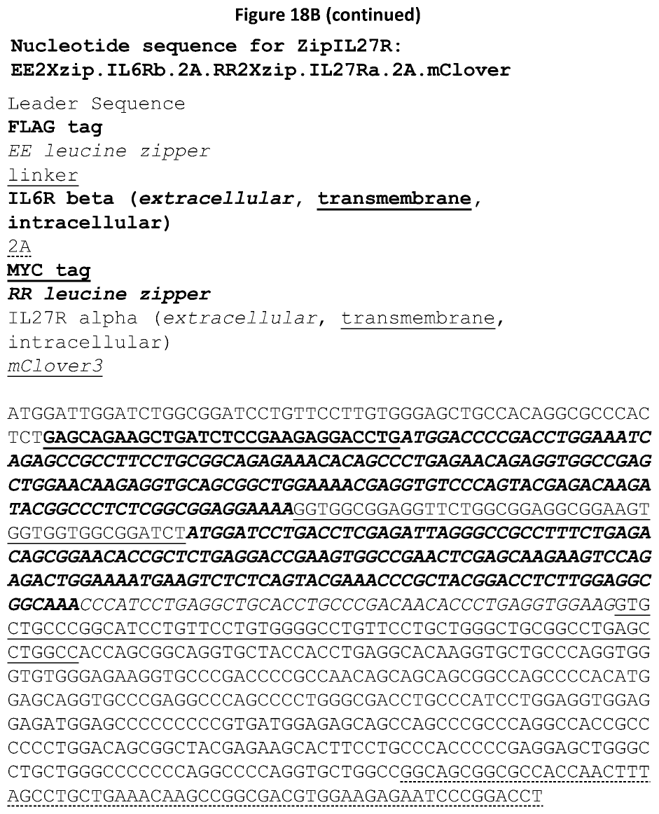

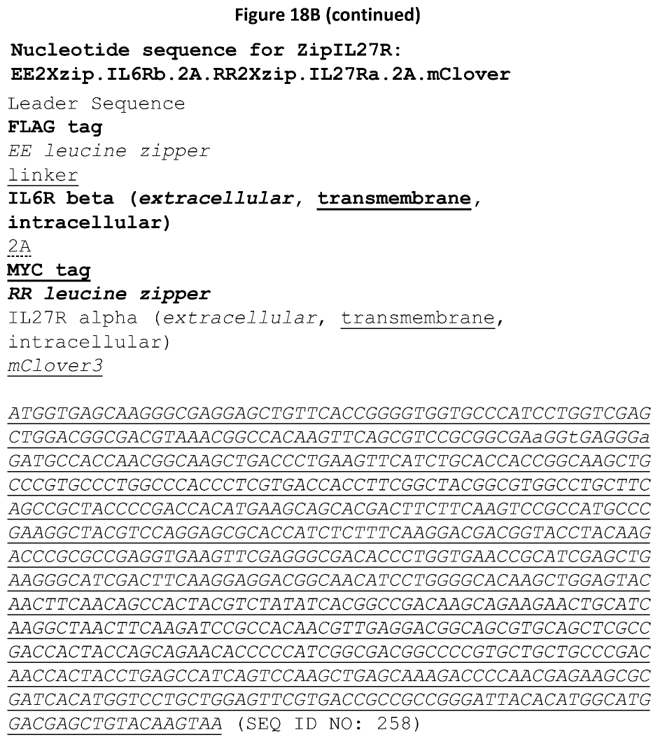

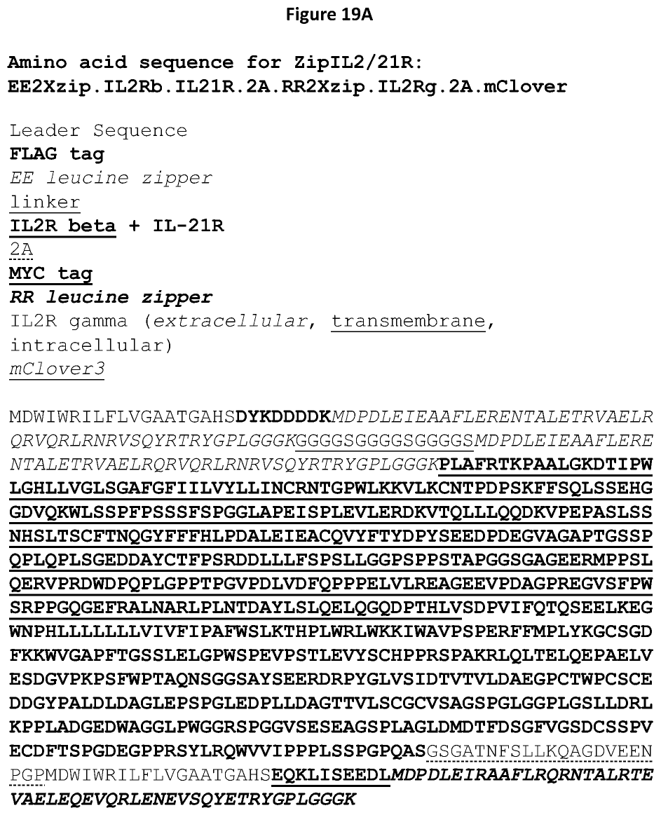

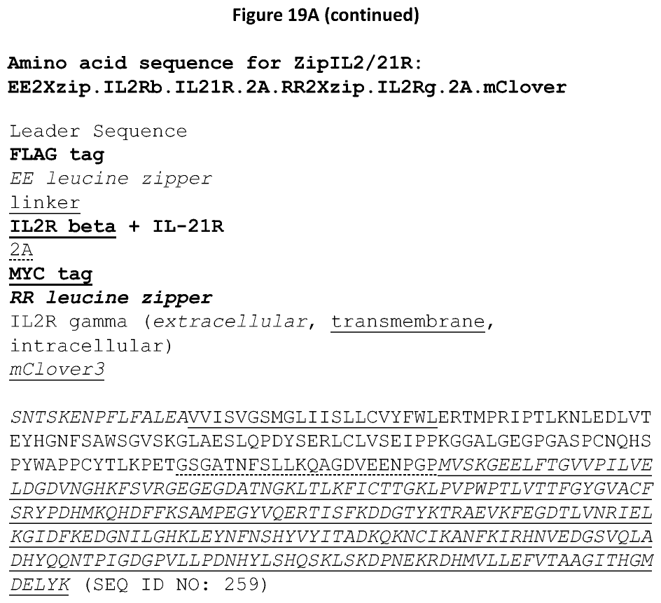

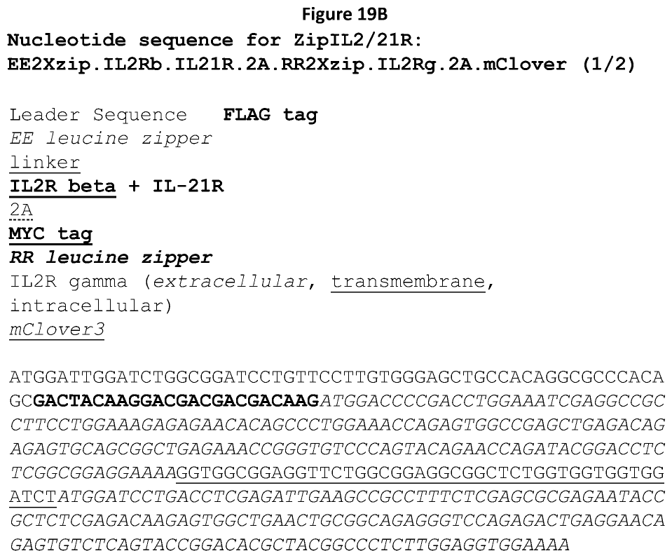

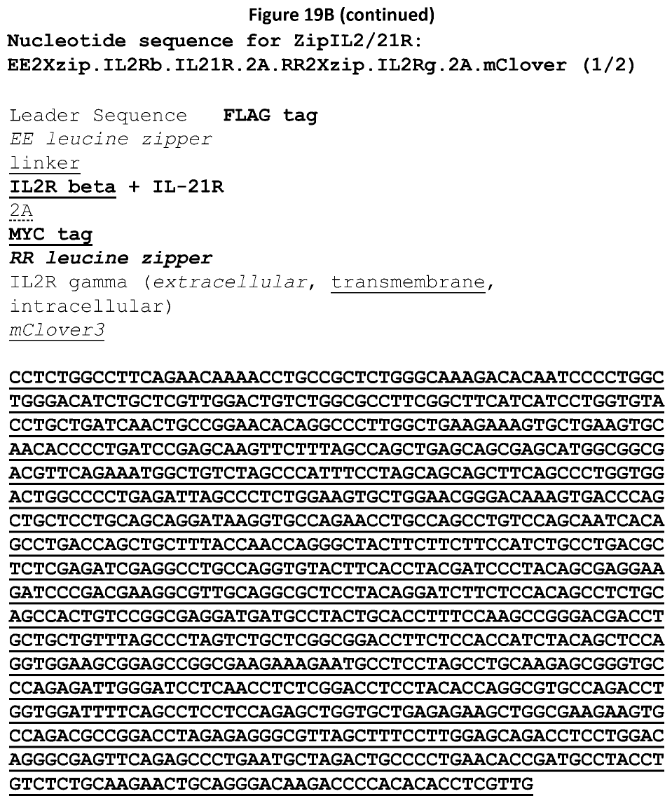

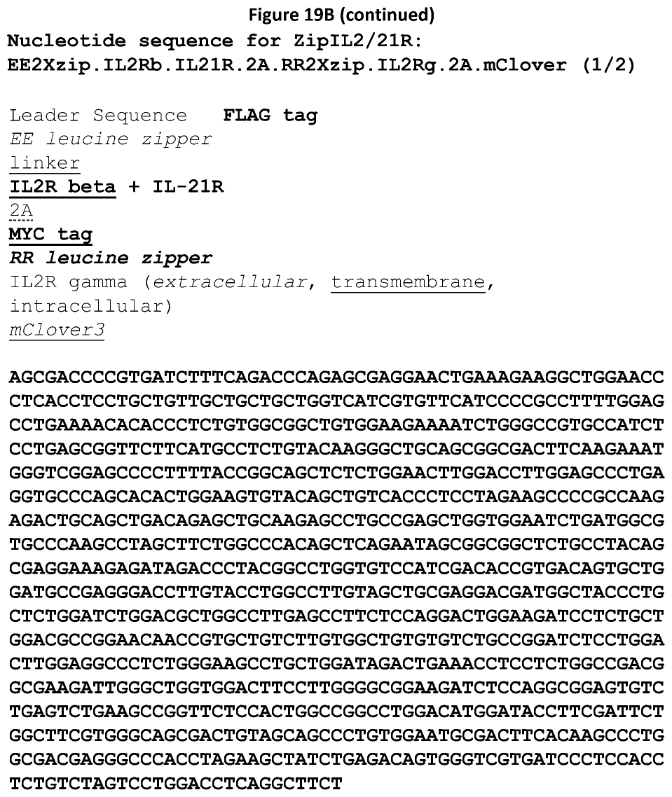

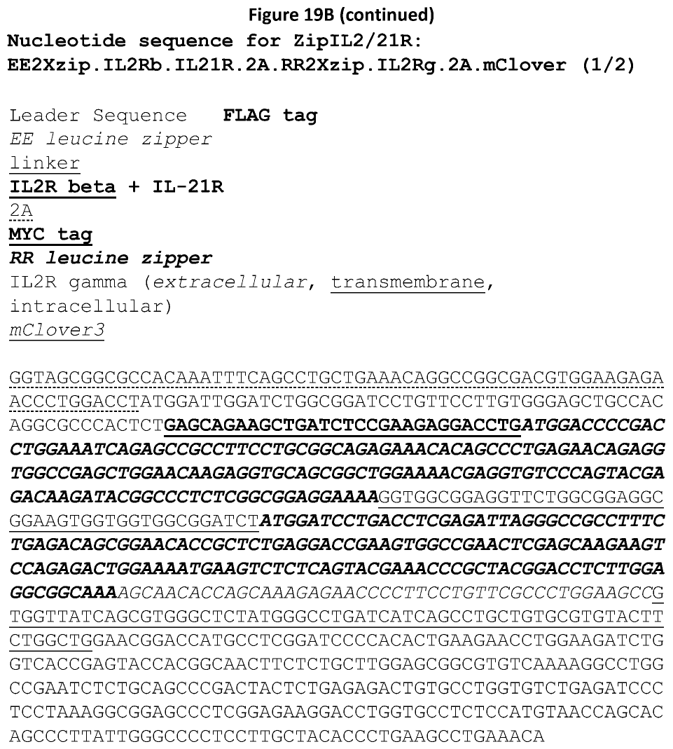

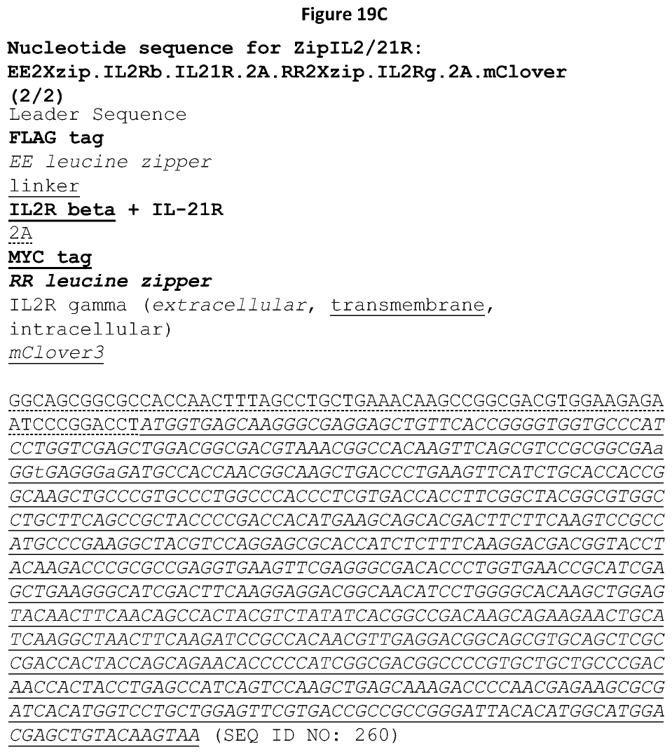

Primary human natural killer (NK) cells can also be modified to express zipReceptors, which improve survival in cytokine-limited conditions. zipIL-2R, zipIL-7R, and zipIL-21R were efficiently expressed by primary NK cells after transduction with γ retroviral vectors (FIG. 8A). Similar to their activity in human T cells, zipIL-2R and zipIL-7R also induced pSTAT5, while zipIL-21R activated pSTAT3 at baseline in transduced NK cells (FIG. 8B). To demonstrate the functional activity of these zipReceptors, transduced NK cells were starved of cytokines for seven days and cell viability was determined by cell surface expression of Annexin V and exclusion of a cell viability dye via flow cytometry. While most NT cells were dead (Annexin V+ and viability dye+), a significant percentage of zipIL-2R and zipIL-7R expressing cells were alive (Annexin V− and viability dye−) (FIG. 8C) Interestingly, zipIL-21R did not improve cell survival in these conditions. However, zipIL-21R may provide benefits to NK cells in other situations or in combination with various stimuli.

In summary, the data provided in FIGS. 6-8 demonstrate that it is feasible to generate functional zipReceptors with different cytokine signaling chains, highlighting that the developed system is modular, and allows for tailored reprogramming of immune cells.

Below are the methods used in the Examples described above.

### Tumor Cell Lines.

The A549 (lung cancer) cell line was purchased from ATCC. The LM7 (osteosarcoma) cell line was provided by Dr. Eugenie Kleinerman (MD Anderson Cancer Center, Houston, TX) [39]. The generation of A549 cells genetically modified to express an enhanced green fluorescent protein firefly luciferase molecule (A549.GFP.ffLuc), was previously described [35]. The generation of the B7-H3−/−LM7 cells (LM7 B7-H3 KO) by CRISPR-Cas9 technology was previously described [36] and the same approach to generate B7-H3 KO A549 cells. Once thawed, cell lines were kept in culture for a maximum of 3 months before a new reference vial was thawed. Cell lines were maintained and expanded in Dulbecco's Modified Eagle Medium (GE Healthcare Life Sciences HyClone Laboratories) supplemented with 10% fetal bovine serum (FBS; GE Healthcare Life Sciences HyClone Laboratories) and 2 mmol/L Glutamax (Invitrogen). Cell lines were authenticated by the ATCC human short-tandem repeat profiling cell authentication service and routinely checked for mycoplasma by the MycoAlert Mycoplasma Detection Kit (Lonza).

### Generation of Viral Vectors.

The generation of the use lentiviral vector encoding a 2^{nd} generation B7-H3-CAR (MGA271.8αTM.CD28ζ) or ΔCAR was previously described [36]. The SFG retroviral vectors encoding IL-2Rβ.1X, IL-2Rβ.2X, IL-2Rγ.1X, IL-2Rγ.2X, zipIL-2R, zipIL-7R, and zipIL-21R were generated by synthesizing gene fragments (Thermo Fisher Scientific) and In-Fusion cloning (Takara Bio). They consisted of (i) either one or two leucine zipper motifs linked by a glycine/serine linker; (ii) 15 amino acids from the extracellular domain of the indicated cytokine receptor chain; (iii) the entire transmembrane and intracellular domain from the indicated receptor chain; (iv) a P2A sequence; and (v) either mRuby or mClover. The sequences of the final constructs were verified by sequencing (Hartwell Center, St. Jude Children's Research Hospital). RD114-pseudotyped retroviral particles were generated by transient transfection of 293T cells as previously described [37]. Supernatants were collected after 48 hours, filtered, and snap-frozen.

### Generation of B7-H3-CAR T Cells.

Human peripheral blood mononuclear cells (PBMCs) were isolated using Lymphoprep (Abbott Laboratories) from de-identified elutriation chambers of leukapheresis products obtained from St. Jude's donor center. On day 0, CD4⁺ and CD8⁺ T cells were enriched from PBMCs by immunomagnetic separation using CD4 and CD8 microbeads (Miltenyi, Germany), an LS column (Miltenyi), and a MidiMACS separator (Miltenyi). Enriched T cells were resuspended at 1×10⁶ cells/mL in RPMI 1640 (GE Healthcare) supplemented with 10% FBS (GE Healthcare), 1% GlutaMAX (Thermo Fisher Scientific), and cytokines IL-7 and IL-15 (10 ng/mL and 5 ng/mL respectively) (Biological Resources Branch, National Cancer Institute, Frederick, MD, USA, and PeproTech, Rocky Hill, NJ, USA) and stimulated overnight on 24-well non-tissue culture-treated plates that were precoated with CD3 and CD28 antibodies (Miltenyi). Transduction was performed on day 1 by adding VSVG-pseudotyped LV particles at an multiplicity of infection (MOI) of 50, and protamine sulfate at 4 μg/mL. On day 4, T cells were transferred into new 24-well tissue culture-treated plates and subsequently expanded with IL-7 and IL-15. All experiments were performed 7-14 days post-transduction. Biological replicates were performed using PBMCs from different healthy donors.

Generation of zipReceptor Transduced T Cells.

To transduce T cells with the B7-H3-CAR and a zipReceptor, T cells were transduced as described in the above section Generation of B7-H3-CAR T Cells and 24 hours after lentiviral transduction, CAR T cells were transferred to retronectin-coated (Clontech) plates with retroviral particles for 2 to 4 days. To transduce T cells with only a zipReceptor, PBMCs were stimulated on plates coated with anti-CD3 and anti-CD28 for 48 hours. Recombinant human IL7 (10 ng/mL, Peprotech) and IL15 (5 ng/mL, Peprotech) were added 24 hours after initial stimulation and were maintained in culture until functional studies were performed. Cells were then seeded onto retronectin-coated (Clontech) plates with retroviral particles for 2 to 4 days. T cells were the transferred into new 24-well tissue culture-treated plates and subsequently expanded with IL-7 and IL-15. Non-transduced T cells were prepared similarly, except that no retrovirus was included in the retronectin wells. All experiments were performed 7-14 days post-transduction. Non-transduced (NT) T cells were prepared similarly, except that no retrovirus was included in the retronectin wells. Biological replicates were performed using PBMCs from different healthy donors.

Generation of zipReceptor Transduced NK Cells.

Human peripheral blood mononuclear cells (PBMCs) were obtained from whole blood of healthy donors under an institutional review board (IRB) approved protocol at St. Jude, after informed consent was obtained in accordance with the Declaration of Helsinki or from de-identified elutriation chambers of leukapheresis products obtained from St. Jude's donor center. Donors were less than haplo-identically matched by human leukocyte antigen (HLA) typing. Cells were subjected to ACK (Ammonium-Chloride-Potassium) Red Blood cell lysis and Ficcol Hypaque (Sigma-Aldrich, St. Louis, MO, USA) gradient separation. Cellular subtype analysis was performed with BD whole blood analysis kit on a BD Lyric flow cytometer (Becton-Dickinson, Franklin Lakes, NJ, USA). PBMCs were depleted of CD4 and CD8 using a standard MACS® magnetic beads. Cells were aliquoted in freezing media with 10% DMSO at 1×10⁷ cells per mL and stored in liquid nitrogen vapor phase until use. 150 Gray cesium-irradiated feeder cells were added to thawed CD4/8-depleted PBMCs at a ratio of 5-10:1 feeder to NK cells. Cells were grown in Stemcell Genix (20802-0500, Cellgenix, Portsmouth, MA, USA) growth media with 20% FBS and 10 units per mL of IL-2, (Peprotech, Rocky Hill, NJ, USA). After 5-7 days, cells were phenotyped and used for downstream experiments. Genetically modified NK cells were generated as follows: Gamma retroviruses encoding zipReceptor constructs were spun at 2000g and bound to retronectin (Takara Bio) coated non-tissue culture 24-well plates for 90 minutes. Supernatant was removed and 250,000 NK cells were seeded per well in a volume of 1 mL of complete growth media. NK cells were incubated for 24 hours and then removed and cultured with 2 mL of complete growth media. NK cells where assessed for transgene expression 48 hours later.

### Feeder Cells.

K562 feeder cells with modified membrane bound interleukin (IL)-15 and 4-1BB ligand [38]were a generous gift from Dr. Dario Campana (National University of Singapore) and grown in Iscove's Modified Dulbecco's Medium (IMDM) media with 10% fetal bovine serum (FBS; Hyclone Laboratories, Chicago, IL, USA).

### Flow Cytometry.

A FACSCanto II and FACSSymphony A5 (BD Biosciences) instruments were used to acquire flow cytometry data, which was analyzed using FlowJo v10 (BD Biosciences). For surface staining, samples were washed with and stained in PBS (Lonza) with 1% FBS (GE Healthcare). For all experiments, matched isotypes or known negatives (e.g., NT T cells) served as gating controls. CAR detection was performed using F(ab′)2 fragment-specific antibody (polyclonal, Jackson ImmunoResearch, West Grove, PA, USA). T cells were stained with fluorochrome-conjugated antibodies using combinations of the following markers: CD4 (clone SK3, BD Biosciences), CD8 (clone SK1, BD Biosciences), CCR7 (clone 2-L1-A, BD Biosciences), and CD45RO (clone HI100, BD Biosciences). Tumor cell lines were evaluated for expression of B7-H3 using B7-H3 antibody (clone 7-517, BD Biosciences, or clone FM276, Miltenyi).

### Intracellular Staining:

Intracellular staining to detect pSTAT5/3 was performed using BD Phosflow Protocol III. Briefly, T cells were unstimulated or stimulated with 5 ng/mL IL-15 for 15 min, then lysed/fixed with 1X Lyse/Fix Buffer (BD Biosciences) for 12 minutes at 37° C. Cells were washed with PBS+1% FBS, then permeabilized with pre-chilled Perm Buffer III (BD Biosciences). Cells were then washed three times before staining with antibodies against pSTAT5 (clone 47, BD Biosciences) or pSTAT3 (clone 4/P, BD Biosciences).

### Sorting:

For in vivo studies, cells were sorted on a BD FACS Aria III cell sorter (BD Biosciences). Cells were stained for B7-H3-CAR expression and mClover was used for detection of zipReceptor transduced cells. DAPI (4′,6-diamidino-2-phenylindole, Thermo Fisher) was used as a viability indicator. Post sorting, cells were cultured for 48 to 72 hours in RPMI containing 20% FBS, 25 μg/mL gentamicin (Gibco), 1X penicillin-streptomycin (Gibco), and 1.5 μg/mL amphotericin B (Gibco) prior to in vivo studies.

### Cytokine Starvation.

To measure cell survival after 7 to 21 days of cytokine withdrawal, 1×10⁶ cells were plated in 1 mL complete RPMI without cytokines in tissue culture treated 48-well plates. After 7-21 days, cells were collected and stained for flow cytometry. To determine apoptosis, T cells were labeled with annexin V (BD Biosciences) and eBioscience Fixable viability dye (Invitrogen).

### MTS Assay.

A CellTiter96 AQueous One Solution Cell Proliferation Assay (Promega) was utilized to assess CAR T-cell cytotoxicity. In a tissue culture-treated 96-well plate, 3,000 A549 or 20,000 LM7 cells were cocultured with serial dilutions of CAR T cells. Media only and tumor cells alone served as controls. Each condition was plated in technical triplicates. After 24 hours, the media and T cells were removed by gently pipetting up and down to avoid disrupting adherent tumor cells. CellTiter96 AQueous One Solution Reagent (phenazine ethosulfate) in complete RPMI was added to each well and incubated at 37° C. for 2 hours. The absorbance at 492 nm was measured using an Infinite 200 Pro MPlex plate reader (Tecan) to quantify the viable cells in each well. Percent live tumor cells was determined by the following formula: (sample-media only)/(tumor alone-media only)×100.

### Repeat Stimulation Assay.

1×10⁶ T cells were co-cultured in complete RPMI with 5×10⁵ tumor cells in a 24-well tissue culture-treated plate. Cells were fed with fresh complete RPMI and split as needed. After 7 days, T cells were harvested, counted, and replated at the same ratio with fresh tumor cells as long as they had killed tumor cells, as determined by microscopic inspection.

### Xenograft Mouse Models.

All animal experiments were approved by the St. Jude Children's Research Hospital Institutional Animal Care and Use Committee. Xenograft experiments were performed with 7- to 10-week-old NSG mice obtained from St. Jude Children's Research Hospital NSG colony. Mice were injected intravenously (i.v.) with 2×10⁶ A549.GFP.ffluc cells in sterile PBS. On day 7, mice received a single i.v. dose of 3×10⁵ B7-H3-CAR or B7-H3-CAR+zipIL-2R T cells in sterile PBS. Injection of sterile PBS only was used as a control. Tumor growth was monitored by weekly bioluminescence imaging (BLI) and mice were euthanized following two sequential BLI measurements >1×10¹⁰ or when they met physical euthanasia criteria (significant weight loss, signs of distress).

### Bioluminescence Imaging.

Mice were injected intraperitoneally (i.p.) with 150 mg/kg of D-luciferin 5 to 10 minutes before imaging, anesthetized with isoflurane, and imaged with a Xenogen IVIS-200 imaging system. The photons emitted from the luciferase-expressing tumor cells were quantified using Living Image software (Caliper Life Sciences). Mice were imaged once per week to track either T cells (EPHA2-CAR T-cell experiments) or LM7 tumor burden (HER2-CAR T-cell experiments).

### Statistical Analysis.

For all experiments, the number of biological replicates and statistical analysis used are described in the figure legends. BLI data were log-transformed before analysis. For comparisons between two groups, a two-tailed t-test was used. For comparisons between three or more groups, analysis of variance (ANOVA) with Dunnett or Tukey post-test was used. Survival was assessed by the log-rank test with Bonferroni adjustment for multiple comparisons. Statistical analyses were conducted with GraphPad Prism software.

### REFERENCES

1. June C H, Sadelain M. Chimeric Antigen Receptor Therapy. New Engl J Med (2018) 379(1):64-73. doi: 10.1056/NEJMra1706169.
2. Srivastava S, Riddell S R. Engineering CAR-T Cells: Design Concepts. Trends Immunol (2015) 36(8):494-502. doi: 10.1016/j.it.2015.06.004.
3. Dotti G, Gottschalk S, Savoldo B, Brenner M K. Design and Development of Therapies Using Chimeric Antigen Receptor-Expressing T Cells. Immunol Rev (2014) 257(1):107-26. doi: 10.1111/imr.12131.
4. Maude S L, Laetsch T W, Buechner J, Rives S, Boyer M, Bittencourt H, et al. Tisagenlecleucel in Children and Young Adults With B-Cell Lymphoblastic Leukemia. New Engl J Med (2018) 378(5):439-48. doi: 10.1056/NEJMoa1709866.
5. Schuster S J, Bishop M R, Tam C S, Waller E K, Borchmann P, McGuirk J P, et al. Tisagenlecleucel in Adult Relapsed or Refractory Diffuse Large B-Cell Lymphoma. New Engl J Med (2019) 380(1):45-56. doi: 10.1056/NEJMoa1804980.
6. Locke F L, Ghobadi A, Jacobson C A, Miklos D B, Lekakis L J, Oluwole 00, et al. Long-Term Safety and Activity of Axicabtagene Ciloleucel in Refractory Large B-Cell Lymphoma (ZUMA-1): A Single-Arm, Multicentre, Phase 1-2 Trial. Lancet Oncol (2019) 20(1):31-42. doi: 10.1016/S1470-2045(18)30864-7.
7. Raje N, Berdeja J, Lin Y, Siegel D, Jagannath S, Madduri D, et al. Anti- BCMA CAR T-Cell Therapy bb2121 in Relapsed or Refractory Multiple Myeloma. New Engl J Med (2019) 380(18):1726-37. doi: 10.1056/NEJMoa1817226.
8. Fry T J, Shah N N, Orentas R J, Stetler-Stevenson M, Yuan C M, Ramakrishna S, et al. CD22-Targeted CAR T Cells Induce Remission in B-ALL That Is Naive or Resistant to CD19-targeted CAR Immunotherapy. Nat Med (2018) 24(1):20-8. doi: 10.1038/nm.4441.
9. Till B G, Jensen M C, Wang J, Qian X, Gopal A K, Maloney D G, et al. CD20− Specific Adoptive Immunotherapy for Lymphoma Using a Chimeric Antigen Receptor With Both CD28 and 4-1BB Domains: Pilot Clinical Trial Results. Blood (2012) 119(17):3940-50. doi: 10.1182/blood-2011-10-387969.
10. Ramos C A, Grover N S, Beaven A W, Lulla P D, Wu M F, Ivanova A, et al. Anti-CD30 CAR-T Cell Therapy in Relapsed and Refractory Hodgkin Lymphoma. J Clin Oncol: Off J Am Soc Clin Oncol (2020) 38(32):3794-804. doi: 10.1200/JCO.20.01342.
11. Mueller K T, Maude S L, Porter D L, Frey N, Wood P, Han X, et al. Cellular Kinetics of CTL019 in Relapsed/Refractory B-Cell Acute Lymphoblastic Leukemia and Chronic Lymphocytic Leukemia. Blood (2017) 130(21):2317-25. doi: 10.1182/blood-2017-06-786129.
12. Heczey A, Louis C U, Savoldo B, Dakhova O, Durett A, Grilley B, et al. Car T Cells Administered in Combination With Lymphodepletion and PD-1 Inhibition to Patients With Neuroblastoma. Mol Ther: J Am Soc Gene Ther (2017) 25(9):2214-24. doi: 10.1016/j.ymthe.2017.05.012.
13. Louis C U, Savoldo B, Dotti G, Pule M, Yvon E, Myers G D, et al. Antitumor Activity and Long-Term Fate of Chimeric Antigen Receptor-Positive T Cells in Patients With Neuroblastoma. Blood (2011) 118(23):6050-6. doi: 10.1182/blood-2011-05-354449.
14. Ahmed N, Brawley V S, Hegde M, Robertson C, Ghazi A, Gerken C, et al. Human Epidermal Growth Factor Receptor 2 (Her2) -Specific Chimeric Antigen Receptor-Modified T Cells for the Immunotherapy of HER2-Positive Sarcoma. J Clin Oncol: Off J Am Soc Clin Oncol (2015) 33 (15):1688-96. doi: 10.1200/JCO.2014.58.0225.
15. Lamers C H, Klaver Y, Gratama J W, Sleijfer S, Debets R. Treatment of Metastatic Renal Cell Carcinoma (mRCC) With CAIX CAR-Engineered T- Cells-a Completed Study Overview. Biochem Soc Trans (2016) 44(3):951-9. doi: 10.1042/BST20160037.
16. Thistlethwaite F C, Gilham D E, Guest R D, Rothwell D G, Pillai M, Burt D J, et al. The Clinical Efficacy of First-Generation Carcinoembryonic Antigen (CEACAM5)-Specific CAR T Cells is Limited by Poor Persistence and Transient Pre-Conditioning-Dependent Respiratory Toxicity. Cancer Immunol Immunother: CII (2017) 66(11):1425-36. doi: 10.1007/s00262-017-2034-7.
17. Hudecek M, Sommermeyer D, Kosasih P L, Silva-Benedict A, Liu L, Rader C, et al. The Nonsignaling Extracellular Spacer Domain of Chimeric Antigen Receptors is Decisive for In Vivo Antitumor Activity. Cancer Immunol Res (2015) 3(2):125-35. doi: 10.1158/2326-6066.CIR-14-0127.
18. Hudecek M, Lupo-Stanghellini M T, Kosasih P L, Sommermeyer D, Jensen M C, Rader C, et al. Receptor Affinity and Extracellular Domain Modifications Affect Tumor Recognition by ROR1-Specific Chimeric Antigen Receptor T Cells. Clin Cancer Res (2013) 19(12):3153-64. doi: 10.1158/1078-0432.CCR-13-0330.
19. Eyquem J, Mansilla-Soto J, Giavridis T, van der Stegen S J, Hamieh M, Cunanan K M, et al. Targeting a CAR to the TRAC Locus With CRISPR/Cas9 Enhances Tumour Rejection. Nature (2017) 543(7643):113-7. doi: 10.1038/nature21405.
20. Feucht J, Sun J, Eyquem J, Ho Y J, Zhao Z, Leibold J, et al. Calibration of CAR Activation Potential Directs Alternative T Cell Fates and Therapeutic Potency. Nat Med (2019) 25(1):82-8. doi: 10.1038/s41591-018-0290-5.
21. Guedan S, Madar A, Casado-Medrano V, Shaw C, Wing A, Liu F, et al. Single Residue in CD28-Costimulated CAR-T Cells Limits Long-Term Persistence and Antitumor Durability. J Clin Invest (2020) 130(6):3087-97. doi: 10.1172/JC1133215.
22. Rupp L J, Schumann K, Roybal K T, Gate R E, Ye C J, Lim W A, et al. CRISPR/Cas9-Mediated PD-1 Disruption Enhances Anti-Tumor Efficacy of Human Chimeric Antigen Receptor T Cells. Sci Rep (2017) 7(1):737. doi: 10.1038/s41598-017-00462-8.
23. Brocker T, Karjalainen K. Signals Through T Cell Receptor-Zeta Chain Alone are Insufficient to Prime Resting T Lymphocytes. JExpMed (1995) 181 (5):1653-9. doi: 10.1084/jem.181.5.1653.
24. Krause A, Guo H F, Latouche J B, Tan C, Cheung N K, Sadelain M. Antigen- Dependent CD28 Signaling Selectively Enhances Survival and Proliferation in Genetically Modified Activated Human Primary T Lymphocytes. JExpMed (1998) 188(4):619-26. doi: 10.1084/jem.188.4.619.
25. Curtsinger J M, Valenzuela J O, Agarwal P, Lins D, Mescher M E. Type I Ifns Provide a Third Signal to CD8 T Cells to Stimulate Clonal Expansion and Differentiation. J Immunol (2005) 174(8):4465-9. doi: 10.4049/jimmunol. 174.8.4465.
26. Mata M, Gerken C, Nguyen P, Krenciute G, Spencer D M, Gottschalk S. Inducible Activation of MyD88 and CD40 in CAR T Cells Results in Controllable and Potent Antitumor Activity in Preclinical Solid Tumor Models. Cancer Discov (2017) 7(11):1306-19. doi: 10.1158/2159-8290.CD17- 0263.
27. Musso T, Calosso L, Zucca M, Millesimo M, Ravarino D, Giovarelli M, et al. Human Monocytes Constitutively Express Membrane-Bound, Biologically Active, and Interferon-Gamma-Upregulated Interleukin-15. Blood (1999) 93 (10):3531-9. doi: 10.1182/blood.V93.10.3531.410k32_3531_3539.
28. Trinchieri G. Interleukin-12 and the Regulation of Innate Resistance and Adaptive Immunity. Nat Rev Immunol (2003) 3(2):133-46. doi: 10.1038/nril001.
29. Kagoya Y, Tanaka S, Guo T, Anczurowski M, Wang C H, Saso K, et al. A Novel Chimeric Antigen Receptor Containing a JAK-STAT Signaling Domain Mediates Superior Antitumor Effects. Nat Med (2018) 24(3):352-9. doi: 10.1038/nm.4478.
30. Zhang H, Zhao H, He X, Xi F, Liu J. Jak-Stat Domain Enhanced MUC1-CAR-T Cells Induced Esophageal Cancer Elimination. Cancer Manag Res (2020) 12:9813-24. doi: 10.2147/CMAR.S264358.
31. Cho, J. H., Collins, J. J. & Wong, W. W. Universal Chimeric Antigen Receptors for Multiplexed and Logical Control of T Cell Responses. Cell 173, 1426-1438 el411, doi:10.1016/j.cell.2018.03.038 (2018).
32. Stuhlmann-Laeisz, C. et al. Forced dimerization of gp130 leads to constitutive STAT3 activation, cytokine-independent growth, and blockade of differentiation of embryonic stem cells. Mol Biol Cell 17, 2986-2995, doi:10.1091/mbc.e05-12-1129 (2006).
33. Jumper, J. et al. Highly accurate protein structure prediction with AlphaFold. Nature 596, 583-589, doi:10.1038/s41586-021-03819-2 (2021).
34. Jaskolski, M. 3D domain swapping, protein oligomerization, and amyloid formation. Acta Biochim Pol 48, 807-827 (2001).
35. Kakarla, S. et al. Antitumor effects of chimeric receptor engineered human T cells directed to tumor stroma. Molecular therapy: the journal of the American Society of Gene Therapy 21, 1611-1620, doi:10.1038/mt.2013.110 (2013).
36. Nguyen, P. et al. Route of 41BB/41BBL Costimulation Determines Effector Function of B7-H3-CAR.CD28zeta T Cells. Mol Ther Oncolytics 18, 202-214, doi:10.1016/j.omto.2020.06.018 (2020).
37. Yi, Z., Prinzing, B. L., Cao, F., Gottschalk, S. & Krenciute, G. Optimizing EphA2-CAR T Cells for the Adoptive Immunotherapy of Glioma. Mol Ther Methods Clin Dev 9, 70-80, doi:10.1016/j.omtm.2018.01.009 (2018).
38. Imai, C., Iwamoto, S. & Campana, D. Genetic modification of primary natural killer cells overcomes inhibitory signals and induces specific killing of leukemic cells. Blood 106, 376-383, doi:10.1182/blood-2004-12-4797 (2005).
39. Jia S-F, Worth L L, Kleinerman E S. A nude mouse model of human osteosarcoma lung metastases for evaluating new therapeutic strategies. Clin Exp Metastasis 1999; 17:501-6.
40. Seldeen K L, McDonald C B, Deegan B J, Bhat V, Farooq A. Dissecting the role of leucine zippers in the binding of bZIP domains of Jun transcription factor to DNA. Biochemical and Biophysical Research Communications, 21 Mar. 2010, 394(4):1030-1035 DOI: 10.1016/j.bbrc.2010.03.116 PMID: 20331972 PMCID: PMC2860604.
41. Moll J R, Ruvinov S B, Pastan I, Vinson C. Designed heterodimerizing leucine zippers with a ranger of pIs and stabilities up to 10(-15) M. Protein Sci. 2001 March; 10(3):649-55. doi: 10.1110/ps.39401.
42. Reinke A W, Baek J, Ashenberg 0, and Keating A E. Networks of bZIP protein-protein interactions diversified over a billion years of evolution. Science. 2013 May 10; 340(6133):730-4. doi: 10.1126/science.1233465.
43. Newman JRS and Keating A E. Comprehensive identification of human bZIP interactions with coiled-coil arrays. Science. 2003 Jun. 27; 300(5628):2097-101. doi: 10.1126/science.1084648. Epub 2003 Jun 12.
44. Monteys A M, Hundley A A, Ranum P T, Tecedor L, Muehlmatt A, Lim E, Lukashev D, Sivasankaran R, Davidson B L. Regulated control of gene therapies by drug-induced splicing. Nature. 2021 August; 596(7871):291-295. doi: 10.1038/s41586-021-03770-2. Epub 2021 Jul 28.
45. Wu C-Y, Roybal K T, Puchner E M, Onuffer J, Lim W A. Remote control of therapeutic T cells through a small molecule-gated chimeric receptor. Science. 2015 Oct. 16; 350(6258):aab4077. doi: 10.1126/science.aab4077. Epub 2015 Sep. 24.
46. Juillerat A, Tkach F, Busser B W, Temburni S, Valton J, Duclert A, Poirot L, Depil S, and Duchateau P. Modulation of chimeric antigen receptor surface expression by a small molecule switch. BMC Biotechnol. 2019; 19: 44. Published online 2019 Jul 3. doi: 10.1186/s12896-019-0537-3.
47. Carbonneau S, Sharma S, Peng L, Rajan V, Hainzl D, Henault M, Yang C, Hale J, Shulok J, Tallarico J, Porter J, Brogdon J L, Dranoff G, Bradner J E, Hild M, and Guimaraes C P. An IMiD-inducible degron provides reversible regulation for chimeric antigen receptor expression and activity. Cell Chemical Biology. Volume 28, Issue 6, 17 Jun. 2021, Pages 802-812.e6
48. Jan M, Scarfo I, Larson R C,Walker A, Schmidts A, Guirguis A A, Gasser J A, Slabicki M, Bouffard A B, Castano A P, Kann M C, Cabral M L, Tepper A, Grinshpun D E, Sperling A S, Kyung T, Sievers Q L, Birnbaum M E, Maus M V, Ebert B L. Reversible ON- and OFF-switch chimeric antigen receptors controlled by lenalidomide. Sci Transl Med. 2021 Jan. 6; 13(575):eabb6295. doi: 10.1126/scitranslmed.abb6295.
49. Zhu H and Kaufman D S. An Improved Method to Produce Clinical-Scale Natural Killer Cells from Human Pluripotent Stem Cells. Methods Mol Biol. 2019; 2048:107-119. doi: 10.1007/978-1-4939-9728-2_12.
50. Iriguchi S, Yasui Y, Kawai Y, Arima S, Kunitomo M, Sato T, Ueda T, Minagawa A, Mishima Y, Yanagawa N, Baba Y, Miyake Y, Nakayama K, Takiguchi M, Shinohara T, Nakatsura T, Yasukawa M, Kassai Y, Hayashi A & Kaneko S. A clinically applicable and scalable method to regenerate T-cells from iPSCs for off-the-shelf T-cell immunotherapy. Nature Communications volume 12, Article number: 430 (2021).

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

List of Sequences

SEQ ID NO: 1 EE leucine zipper motif
LEIEAAFLERENTALETRVAELRQRVQRLRNRVSQYRTRYGPL
SEQ ID NO: 2 RR leucine zipper motif
LEIRAAFLRQRNTALRTEVAELEQEVQRLENEVSQYETRYGPL
SEQ ID NO: 3 Leader
MDWIWRILFLVGAATGAHS
SEQ ID NO: 4 Leader
ATGGATTGGATCTGGCGGATCCTGTTCCTTGTGGGAGCTGCCACAGGCGCCCACAGC
SEQ ID NO: 5 Leader
ATGGATTGGATCTGGCGGATCCTGTTCCTTGTGGGAGCTGCCACAGGCGCCCACTCT
SEQ ID NO: 6 FLAG tag
DYKDDDDK
SEQ ID NO: 7 FLAG tag
GACTACAAGGACGACGACGACAAG
SEQ ID NO: 8 EE leucine zipper
MDPDLEIEAAFLERENTALETRVAELRQRVQRLRNRVSQYRTRYGPLGGGK
SEQ ID NO: 9 EE leucine zipper
ATGGACCCCGACCTGGAAATCGAGGCCGCCTTCCTGGAAAGAGAGAACACAGCCCT
GGAAACCAGAGTGGCCGAGCTGAGACAGAGAGTGCAGCGGCTGAGAAACCGGGTG
TCCCAGTACAGAACCAGATACGGACCTCTCGGCGGAGGAAAA
SEQ ID NO: 10 EE leucine zipper
ATGGATCCTGACCTCGAGATTGAAGCCGCCTTTCTCGAGCGCGAGAATACCGCTCTC
GAGACAAGAGTGGCTGAACTGCGGCAGAGGGTCCAGAGACTGAGGAACAGAGTGT
CTCAGTACCGGACACGCTACGGCCCTCTTGGAGGTGGAAAA
SEQ ID NO: 11 linker
GGGGSGGGGSGGGGS
SEQ ID NO: 12 linker
GGTGGCGGAGGTTCTGGCGGAGGCGGCTCTGGTGGTGGTGGATCT
SEQ ID NO: 13 linker
GGTGGCGGAGGTTCTGGCGGAGGCGGAAGTGGTGGTGGCGGATCT
SEQ ID NO: 14 IL2R beta extracellular(hinge)
PLAFRTKPAALGKDT
SEQ ID NO: 15 IL2R beta extracellular(hinge)
CCTCTGGCCTTCAGAACAAAACCTGCCGCTCTGGGCAAAGACACA
SEQ ID NO: 16 IL2R beta transmembrane
IPWLGHLLVGLSGAFGFIILVYLLI
SEQ ID NO: 17 IL2R beta transmembrane
ATCCCCTGGCTGGGACATCTGCTCGTTGGACTGTCTGGCGCCTTCGGCTTCATCATCC
TGGTGTACCTGCTGATC
SEQ ID NO: 18 IL2R beta intracellular
NCRNTGPWLKKVLKCNTPDPSKFFSQLSSEHGGDVQKWLSSPFPSSSFSPGGLAPEISPLE
VLERDKVTQLLLQQDKVPEPASLSSNHSLTSCFTNQGYFFFHLPDALEIEACQVYFTYDP
YSEEDPDEGVAGAPTGSSPQPLQPLSGEDDAYCTFPSRDDLLLFSPSLLGGPSPPSTAPGG
SGAGEERMPPSLQERVPRDWDPQPLGPPTPGVPDLVDFQPPPELVLREAGEEVPDAGPR
EGVSFPWSRPPGQGEFRALNARLPLNTDAYLSLQELQGQDPTHLV
SEQ ID NO: 19 IL2R beta intracellular
AACTGCCGGAACACAGGCCCTTGGCTGAAGAAAGTGCTGAAGTGCAACACCCCTGA
TCCGAGCAAGTTCTTTAGCCAGCTGAGCAGCGAGCATGGCGGCGACGTTCAGAAAT
GGCTGTCTAGCCCATTTCCTAGCAGCAGCTTCAGCCCTGGTGGACTGGCCCCTGAGA
TTAGCCCTCTGGAAGTGCTGGAACGGGACAAAGTGACCCAGCTGCTCCTGCAGCAG
GATAAGGTGCCAGAACCTGCCAGCCTGTCCAGCAATCACAGCCTGACCAGCTGCTTT
ACCAACCAGGGCTACTTCTTCTTCCATCTGCCTGACGCTCTCGAGATCGAGGCCTGC
CAGGTGTACTTCACCTACGATCCCTACAGCGAGGAAGATCCCGACGAAGGCGTTGC
AGGCGCTCCTACAGGATCTTCTCCACAGCCTCTGCAGCCACTGTCCGGCGAGGATGA
TGCCTACTGCACCTTTCCAAGCCGGGACGACCTGCTGCTGTTTAGCCCTAGTCTGCTC
GGCGGACCTTCTCCACCATCTACAGCTCCAGGTGGAAGCGGAGCCGGCGAAGAAAG
AATGCCTCCTAGCCTGCAAGAGCGGGTGCCCAGAGATTGGGATCCTCAACCTCTCGG
ACCTCCTACACCAGGCGTGCCAGACCTGGTGGATTTTCAGCCTCCTCCAGAGCTGGT
GCTGAGAGAAGCTGGCGAAGAAGTGCCAGACGCCGGACCTAGAGAGGGCGTTAGCT
TTCCTTGGAGCAGACCTCCTGGACAGGGCGAGTTCAGAGCCCTGAATGCTAGACTGC
CCCTGAACACCGATGCCTACCTGTCTCTGCAAGAACTGCAGGGACAAGACCCCACA
CACCTCGTTG
SEQ ID NO: 20 IL2R beta (extracellular, transmembrane, intracellular)
PLAFRTKPAALGKDTIPWLGHLLVGLSGAFGFIILVYLLINCRNTGPWLKKVLKCNTPDP
SKFFSQLSSEHGGDVQKWLSSPFPSSSFSPGGLAPEISPLEVLERDKVTQLLLQQDKVPEP
ASLSSNHSLTSCFTNQGYFFFHLPDALEIEACQVYFTYDPYSEEDPDEGVAGAPTGSSPQP
LQPLSGEDDAYCTFPSRDDLLLFSPSLLGGPSPPSTAPGGSGAGEERMPPSLQERVPRDW
DPQPLGPPTPGVPDLVDFQPPPELVLREAGEEVPDAGPREGVSFPWSRPPGQGEFRALNA
RLPLNTDAYLSLQELQGQDPTHLV
SEQ ID NO: 21 IL2R beta (extracellular, transmembrane, intracellular)
CCTCTGGCCTTCAGAACAAAACCTGCCGCTCTGGGCAAAGACACAATCCCCTGGCTG
GGACATCTGCTCGTTGGACTGTCTGGCGCCTTCGGCTTCATCATCCTGGTGTACCTGC
TGATCAACTGCCGGAACACAGGCCCTTGGCTGAAGAAAGTGCTGAAGTGCAACACC
CCTGATCCGAGCAAGTTCTTTAGCCAGCTGAGCAGCGAGCATGGCGGCGACGTTCA
GAAATGGCTGTCTAGCCCATTTCCTAGCAGCAGCTTCAGCCCTGGTGGACTGGCCCC
TGAGATTAGCCCTCTGGAAGTGCTGGAACGGGACAAAGTGACCCAGCTGCTCCTGC
AGCAGGATAAGGTGCCAGAACCTGCCAGCCTGTCCAGCAATCACAGCCTGACCAGC
TGCTTTACCAACCAGGGCTACTTCTTCTTCCATCTGCCTGACGCTCTCGAGATCGAGG
CCTGCCAGGTGTACTTCACCTACGATCCCTACAGCGAGGAAGATCCCGACGAAGGC
GTTGCAGGCGCTCCTACAGGATCTTCTCCACAGCCTCTGCAGCCACTGTCCGGCGAG
GATGATGCCTACTGCACCTTTCCAAGCCGGGACGACCTGCTGCTGTTTAGCCCTAGT
CTGCTCGGCGGACCTTCTCCACCATCTACAGCTCCAGGTGGAAGCGGAGCCGGCGA
AGAAAGAATGCCTCCTAGCCTGCAAGAGCGGGTGCCCAGAGATTGGGATCCTCAAC
CTCTCGGACCTCCTACACCAGGCGTGCCAGACCTGGTGGATTTTCAGCCTCCTCCAG
AGCTGGTGCTGAGAGAAGCTGGCGAAGAAGTGCCAGACGCCGGACCTAGAGAGGG
CGTTAGCTTTCCTTGGAGCAGACCTCCTGGACAGGGCGAGTTCAGAGCCCTGAATGC
TAGACTGCCCCTGAACACCGATGCCTACCTGTCTCTGCAAGAACTGCAGGGACAAG
ACCCCACACACCTCGTTG
SEQ ID NO: 22 2A
GSGATNFSLLKQAGDVEENPGP
SEQ ID NO: 23 2A
GGTAGCGGCGCCACAAATTTCAGCCTGCTGAAACAGGCCGGCGACGTGGAAGAGAA
CCCTGGACCT
SEQ ID NO: 24 2A
GGCAGCGGCGCCACCAACTTTAGCCTGCTGAAACAAGCCGGCGACGTGGAAGAGAA
TCCCGGACCT
SEQ ID NO: 25 MYC tag
EQKLISEEDL
SEQ ID NO: 26 MYC tag
GAGCAGAAGCTGATCTCCGAAGAGGACCTG
SEQ ID NO: 27 RR leucine zipper
MDPDLEIRAAFLRQRNTALRTEVAELEQEVQRLENEVSQYETRYGPLGGGK
SEQ ID NO: 28 RR leucine zipper
ATGGACCCCGACCTGGAAATCAGAGCCGCCTTCCTGCGGCAGAGAAACACAGCCCT
GAGAACAGAGGTGGCCGAGCTGGAACAAGAGGTGCAGCGGCTGGAAAACGAGGTG
TCCCAGTACGAGACAAGATACGGCCCTCTCGGCGGAGGAAAA
SEQ ID NO: 29 RR leucine zipper
ATGGATCCTGACCTCGAGATTAGGGCCGCCTTTCTGAGACAGCGGAACACCGCTCTG
AGGACCGAAGTGGCCGAACTCGAGCAAGAAGTCCAGAGACTGGAAAATGAAGTCTC
TCAGTACGAAACCCGCTACGGACCTCTTGGAGGCGGCAAA
SEQ ID NO: 30 IL2R gamma extracellular (hinge)
SNTSKENPFLFALEA
SEQ ID NO: 31 IL2R gamma extracellular (hinge)
AGCAACACCAGCAAAGAGAACCCCTTCCTGTTCGCCCTGGAAGCC
SEQ ID NO: 32 IL2R gamma transmembrane
VVISVGSMGLIISLLCVYFWL
SEQ ID NO: 33 IL2R gamma transmembrane
GTGGTTATCAGCGTGGGCTCTATGGGCCTGATCATCAGCCTGCTGTGCGTGTACTTCT
GGCTG
SEQ ID NO: 34 IL2R gamma intracellular
ERTMPRIPTLKNLEDLVTEYHGNFSAWSGVSKGLAESLQPDYSERLCLVSEIPPKGGALG
EGPGASPCNQHSPYWAPPCYTLKPET
SEQ ID NO: 35 IL2R gamma intracellular
GAACGGACCATGCCTCGGATCCCCACACTGAAGAACCTGGAAGATCTGGTCACCGA
GTACCACGGCAACTTCTCTGCTTGGAGCGGCGTGTCAAAAGGCCTGGCCGAATCTCT
GCAGCCCGACTACTCTGAGAGACTGTGCCTGGTGTCTGAGATCCCTCCTAAAGGCGG
AGCCCTCGGAGAAGGACCTGGTGCCTCTCCATGTAACCAGCACAGCCCTTATTGGGC
CCCTCCTTGCTACACCCTGAAGCCTGAAACA
SEQ ID NO: 36 IL2R gamma extracellular, transmembrane, intracellular
SNTSKENPFLFALEAVVISVGSMGLIISLLCVYFWLERTMPRIPTLKNLEDLVTEYHGNFS
AWSGVSKGLAESLQPDYSERLCLVSEIPPKGGALGEGPGASPCNQHSPYWAPPCYTLKP
ET
SEQ ID NO: 37 IL2R gamma extracellular, transmembrane, intracellular
AGCAACACCAGCAAAGAGAACCCCTTCCTGTTCGCCCTGGAAGCCGTGGTTATCAG
CGTGGGCTCTATGGGCCTGATCATCAGCCTGCTGTGCGTGTACTTCTGGCTGGAACG
GACCATGCCTCGGATCCCCACACTGAAGAACCTGGAAGATCTGGTCACCGAGTACC
ACGGCAACTTCTCTGCTTGGAGCGGCGTGTCAAAAGGCCTGGCCGAATCTCTGCAGC
CCGACTACTCTGAGAGACTGTGCCTGGTGTCTGAGATCCCTCCTAAAGGCGGAGCCC
TCGGAGAAGGACCTGGTGCCTCTCCATGTAACCAGCACAGCCCTTATTGGGCCCCTC
CTTGCTACACCCTGAAGCCTGAAACA
SEQ ID NO: 38 mClover3
MVSKGEELFTGVVPILVELDGDVNGHKFSVRGEGEGDATNGKLTLKFICTTGKLPVPWP
TLVTTFGYGVACFSRYPDHMKQHDFFKSAMPEGYVQERTISFKDDGTYKTRAEVKFEG
DTLVNRIELKGIDFKEDGNILGHKLEYNFNSHYVYITADKQKNCIKANFKIRHNVEDGSV
QLADHYQQNTPIGDGPVLLPDNHYLSHQSKLSKDPNEKRDHMVLLEFVTAAGITHGMD
ELYK
SEQ ID NO: 39 mClover3
ATGGTGAGCAAGGGCGAGGAGCTGTTCACCGGGGTGGTGCCCATCCTGGTCGAGCT
GGACGGCGACGTAAACGGCCACAAGTTCAGCGTCCGCGGCGAaGGtGAGGGaGATGC
CACCAACGGCAAGCTGACCCTGAAGTTCATCTGCACCACCGGCAAGCTGCCCGTGC
CCTGGCCCACCCTCGTGACCACCTTCGGCTACGGCGTGGCCTGCTTCAGCCGCTACC
CCGACCACATGAAGCAGCACGACTTCTTCAAGTCCGCCATGCCCGAAGGCTACGTCC
AGGAGCGCACCATCTCTTTCAAGGACGACGGTACCTACAAGACCCGCGCCGAGGTG
AAGTTCGAGGGCGACACCCTGGTGAACCGCATCGAGCTGAAGGGCATCGACTTCAA
GGAGGACGGCAACATCCTGGGGCACAAGCTGGAGTACAACTTCAACAGCCACTACG
TCTATATCACGGCCGACAAGCAGAAGAACTGCATCAAGGCTAACTTCAAGATCCGC
CACAACGTTGAGGACGGCAGCGTGCAGCTCGCCGACCACTACCAGCAGAACACCCC
CATCGGCGACGGCCCCGTGCTGCTGCCCGACAACCACTACCTGAGCCATCAGTCCAA
GCTGAGCAAAGACCCCAACGAGAAGCGCGATCACATGGTCCTGCTGGAGTTCGTGA
CCGCCGCCGGGATTACACATGGCATGGACGAGCTGTACAAGTAA
SEQ ID NO: 40 EE2Xzip.IL2Rb.2A.RR2Xzip.IL2Rg.2A.mClover_IL2Rb chain
MDPDLEIEAAFLERENTALETRVAELRQRVQRLRNRVSQYRTRYGPLGGGKGGGGSGG
GGSGGGGSMDPDLEIEAAFLERENTALETRVAELRQRVQRLRNRVSQYRTRYGPLGGG
KPLAFRTKPAALGKDTIPWLGHLLVGLSGAFGFIILVYLLINCRNTGPWLKKVLKCNTPD
PSKFFSQLSSEHGGDVQKWLSSPFPSSSFSPGGLAPEISPLEVLERDKVTQLLLQQDKVPE
PASLSSNHSLTSCFTNQGYFFFHLPDALEIEACQVYFTYDPYSEEDPDEGVAGAPTGSSPQ
PLQPLSGEDDAYCTFPSRDDLLLFSPSLLGGPSPPSTAPGGSGAGEERMPPSLQERVPRD
WDPQPLGPPTPGVPDLVDFQPPPELVLREAGEEVPDAGPREGVSFPWSRPPGQGEFRAL
NARLPLNTDAYLSLQELQGQDPTHLV
SEQ ID NO: 41 EE2Xzip.IL2Rb.2A.RR2Xzip.IL2Rg.2A.mClover_IL2Rb chain
ATGGACCCCGACCTGGAAATCGAGGCCGCCTTCCTGGAAAGAGAGAACACAGCCCT
GGAAACCAGAGTGGCCGAGCTGAGACAGAGAGTGCAGCGGCTGAGAAACCGGGTG
TCCCAGTACAGAACCAGATACGGACCTCTCGGCGGAGGAAAAGGTGGCGGAGGTTC
TGGCGGAGGCGGCTCTGGTGGTGGTGGATCTATGGATCCTGACCTCGAGATTGAAGC
CGCCTTTCTCGAGCGCGAGAATACCGCTCTCGAGACAAGAGTGGCTGAACTGCGGC
AGAGGGTCCAGAGACTGAGGAACAGAGTGTCTCAGTACCGGACACGCTACGGCCCT
CTTGGAGGTGGAAAACCTCTGGCCTTCAGAACAAAACCTGCCGCTCTGGGCAAAGA
CACAATCCCCTGGCTGGGACATCTGCTCGTTGGACTGTCTGGCGCCTTCGGCTTCAT
CATCCTGGTGTACCTGCTGATCAACTGCCGGAACACAGGCCCTTGGCTGAAGAAAGT
GCTGAAGTGCAACACCCCTGATCCGAGCAAGTTCTTTAGCCAGCTGAGCAGCGAGC
ATGGCGGCGACGTTCAGAAATGGCTGTCTAGCCCATTTCCTAGCAGCAGCTTCAGCC
CTGGTGGACTGGCCCCTGAGATTAGCCCTCTGGAAGTGCTGGAACGGGACAAAGTG
ACCCAGCTGCTCCTGCAGCAGGATAAGGTGCCAGAACCTGCCAGCCTGTCCAGCAA
TCACAGCCTGACCAGCTGCTTTACCAACCAGGGCTACTTCTTCTTCCATCTGCCTGAC
GCTCTCGAGATCGAGGCCTGCCAGGTGTACTTCACCTACGATCCCTACAGCGAGGAA
GATCCCGACGAAGGCGTTGCAGGCGCTCCTACAGGATCTTCTCCACAGCCTCTGCAG
CCACTGTCCGGCGAGGATGATGCCTACTGCACCTTTCCAAGCCGGGACGACCTGCTG
CTGTTTAGCCCTAGTCTGCTCGGCGGACCTTCTCCACCATCTACAGCTCCAGGTGGA
AGCGGAGCCGGCGAAGAAAGAATGCCTCCTAGCCTGCAAGAGCGGGTGCCCAGAG
ATTGGGATCCTCAACCTCTCGGACCTCCTACACCAGGCGTGCCAGACCTGGTGGATT
TTCAGCCTCCTCCAGAGCTGGTGCTGAGAGAAGCTGGCGAAGAAGTGCCAGACGCC
GGACCTAGAGAGGGCGTTAGCTTTCCTTGGAGCAGACCTCCTGGACAGGGCGAGTT
CAGAGCCCTGAATGCTAGACTGCCCCTGAACACCGATGCCTACCTGTCTCTGCAAGA
ACTGCAGGGACAAGACCCCACACACCTCGTTG
SEQ ID NO: 42 EE2Xzip.IL2Rb.2A.RR2Xzip.IL2Rg.2A.mClover_IL2Rg chain
MDPDLEIRAAFLRQRNTALRTEVAELEQEVQRLENEVSQYETRYGPLGGGKGGGGSGG
GGSGGGGSMDPDLEIRAAFLRQRNTALRTEVAELEQEVQRLENEVSQYETRYGPLGGG
KSNTSKENPFLFALEAVVISVGSMGLIISLLCVYFWLERTMPRIPTLKNLEDLVTEYHGNF
SAWSGVSKGLAESLQPDYSERLCLVSEIPPKGGALGEGPGASPCNQHSPYWAPPCYTLK
PET
SEQ ID NO: 43 EE2Xzip.IL2Rb.2A.RR2Xzip.IL2Rg.2A.mClover_IL2Rg chain
ATGGACCCCGACCTGGAAATCAGAGCCGCCTTCCTGCGGCAGAGAAACACAGCCCT
GAGAACAGAGGTGGCCGAGCTGGAACAAGAGGTGCAGCGGCTGGAAAACGAGGTG
TCCCAGTACGAGACAAGATACGGCCCTCTCGGCGGAGGAAAAGGTGGCGGAGGTTC
TGGCGGAGGCGGAAGTGGTGGTGGCGGATCTATGGATCCTGACCTCGAGATTAGGG
CCGCCTTTCTGAGACAGCGGAACACCGCTCTGAGGACCGAAGTGGCCGAACTCGAG
CAAGAAGTCCAGAGACTGGAAAATGAAGTCTCTCAGTACGAAACCCGCTACGGACC
TCTTGGAGGCGGCAAAAGCAACACCAGCAAAGAGAACCCCTTCCTGTTCGCCCTGG
AAGCCGTGGTTATCAGCGTGGGCTCTATGGGCCTGATCATCAGCCTGCTGTGCGTGT
ACTTCTGGCTGGAACGGACCATGCCTCGGATCCCCACACTGAAGAACCTGGAAGAT
CTGGTCACCGAGTACCACGGCAACTTCTCTGCTTGGAGCGGCGTGTCAAAAGGCCTG
GCCGAATCTCTGCAGCCCGACTACTCTGAGAGACTGTGCCTGGTGTCTGAGATCCCT
CCTAAAGGCGGAGCCCTCGGAGAAGGACCTGGTGCCTCTCCATGTAACCAGCACAG
CCCTTATTGGGCCCCTCCTTGCTACACCCTGAAGCCTGAAACA
SEQ ID NO: 44 IL7R alpha extracellular(hinge)
YFRTPEINNSSGEMD
SEQ ID NO: 45 IL7R alpha extracellular(hinge)
TACTTCAGAACCCCTGAGATCAACAACAGCAGCGGCGAGATGGAT
SEQ ID NO: 46 IL7R alpha transmembrane
PILLTISILSFFSVALLVILACVLW
SEQ ID NO: 47 IL7R alpha transmembrane
CCCATCCTGCTGACAATCAGCATCCTGAGCTTTTTCAGCGTGGCCCTGCTGGTCATCC
TGGCCTGTGTGCTGTGG
SEQ ID NO: 48 IL7R alpha intracellular
KKRIKPIVWPSLPDHKKTLEHLCKKPRKNLNVSFNPESFLDCQIHRVDDIQARDEVEGFL
QDTFPQQLEESEKQRLGGDVQSPNCPSEDVVITPESFGRDSSLTCLAGNVSACDAPILSSS
RSLDCRESGKNGPHVYQDLLLSLGTTNSTLPPPFSLQSGILTLNPVAQGQPILTSLGSNQE
EAYVTMSSFYQNQ
SEQ ID NO: 49 IL7R alpha intracellular
AAGAAGCGGATCAAGCCCATCGTGTGGCCCAGCCTGCCTGACCACAAGAAAACCCT
GGAACACCTGTGCAAGAAGCCCCGGAAGAACCTGAACGTGTCCTTCAATCCCGAGA
GCTTCCTGGACTGTCAGATCCACAGAGTGGACGACATCCAGGCCAGGGACGAAGTG
GAAGGCTTTCTGCAGGACACATTCCCTCAGCAGCTGGAAGAGAGCGAGAAGCAGAG
ACTTGGAGGCGACGTGCAGAGCCCTAATTGCCCTTCTGAGGACGTGGTCATCACCCC
AGAGAGCTTCGGCAGAGATAGCAGCCTGACATGTCTGGCCGGCAATGTGTCCGCCT
GTGATGCCCCTATCCTGAGCAGCAGCAGAAGCCTGGATTGCAGAGAGAGCGGCAAG
AACGGCCCTCACGTGTACCAGGATCTGCTCCTGTCTCTGGGCACCACCAATAGCACA
CTGCCTCCACCATTCAGCCTGCAGAGCGGCATCCTGACACTGAACCCTGTTGCTCAG
GGCCAGCCAATCCTGACAAGCCTGGGCAGCAATCAAGAAGAGGCCTACGTCACCAT
GAGCAGCTTCTACCAGAATCAAG
SEQ ID NO: 50 IL7R alpha (extracellular, transmembrane, intracellular)
YFRTPEINNSSGEMDPILLTISILSFFSVALLVILACVLWKKRIKPIVWPSLPDHKKTLEHL
CKKPRKNLNVSFNPESFLDCQIHRVDDIQARDEVEGFLQDTFPQQLEESEKQRLGGDVQ
SPNCPSEDVVITPESFGRDSSLTCLAGNVSACDAPILSSSRSLDCRESGKNGPHVYQDLLL
SLGTTNSTLPPPFSLQSGILTLNPVAQGQPILTSLGSNQEEAYVTMSSFYQNQ
SEQ ID NO: 51 IL7R alpha (extracellular, transmembrane, intracellular)
TACTTCAGAACCCCTGAGATCAACAACAGCAGCGGCGAGATGGATCCCATCCTGCT
GACAATCAGCATCCTGAGCTTTTTCAGCGTGGCCCTGCTGGTCATCCTGGCCTGTGT
GCTGTGGAAGAAGCGGATCAAGCCCATCGTGTGGCCCAGCCTGCCTGACCACAAGA
AAACCCTGGAACACCTGTGCAAGAAGCCCCGGAAGAACCTGAACGTGTCCTTCAAT
CCCGAGAGCTTCCTGGACTGTCAGATCCACAGAGTGGACGACATCCAGGCCAGGGA
CGAAGTGGAAGGCTTTCTGCAGGACACATTCCCTCAGCAGCTGGAAGAGAGCGAGA
AGCAGAGACTTGGAGGCGACGTGCAGAGCCCTAATTGCCCTTCTGAGGACGTGGTC
ATCACCCCAGAGAGCTTCGGCAGAGATAGCAGCCTGACATGTCTGGCCGGCAATGT
GTCCGCCTGTGATGCCCCTATCCTGAGCAGCAGCAGAAGCCTGGATTGCAGAGAGA
GCGGCAAGAACGGCCCTCACGTGTACCAGGATCTGCTCCTGTCTCTGGGCACCACCA
ATAGCACACTGCCTCCACCATTCAGCCTGCAGAGCGGCATCCTGACACTGAACCCTG
TTGCTCAGGGCCAGCCAATCCTGACAAGCCTGGGCAGCAATCAAGAAGAGGCCTAC
GTCACCATGAGCAGCTTCTACCAGAATCAAG
SEQ ID NO: 52 ZipIL7R: EE2Xzip.IL7Ra.2A.RR2Xzip.IL2Rg.2A.mClover_IL7Ra chain
MDPDLEIEAAFLERENTALETRVAELRQRVQRLRNRVSQYRTRYGPLGGGKGGGGSGG
GGSGGGGSMDPDLEIEAAFLERENTALETRVAELRQRVQRLRNRVSQYRTRYGPLGGG
KYFRTPEINNSSGEMDPILLTISILSFFSVALLVILACVLWKKRIKPIVWPSLPDHKKTLEH
LCKKPRKNLNVSFNPESFLDCQIHRVDDIQARDEVEGFLQDTFPQQLEESEKQRLGGDV
QSPNCPSEDVVITPESFGRDSSLTCLAGNVSACDAPILSSSRSLDCRESGKNGPHVYQDLL
LSLGTTNSTLPPPFSLQSGILTLNPVAQGQPILTSLGSNQEEAYVTMSSFYQNQ
SEQ ID NO: 53 ZipIL7R: EE2Xzip.IL7Ra.2A.RR2Xzip.IL2Rg.2A.mClover_IL7Ra chain
ATGGACCCCGACCTGGAAATCGAGGCCGCCTTCCTGGAAAGAGAGAACACAGCCCT
GGAAACCAGAGTGGCCGAGCTGAGACAGAGAGTGCAGCGGCTGAGAAACCGGGTG
TCCCAGTACAGAACCAGATACGGACCTCTCGGCGGAGGAAAAGGTGGCGGAGGTTC
TGGCGGAGGCGGCTCTGGTGGTGGTGGATCTATGGATCCTGACCTCGAGATTGAAGC
CGCCTTTCTCGAGCGCGAGAATACCGCTCTCGAGACAAGAGTGGCTGAACTGCGGC
AGAGGGTCCAGAGACTGAGGAACAGAGTGTCTCAGTACCGGACACGCTACGGCCCT
CTTGGAGGTGGAAAATACTTCAGAACCCCTGAGATCAACAACAGCAGCGGCGAGAT
GGATCCCATCCTGCTGACAATCAGCATCCTGAGCTTTTTCAGCGTGGCCCTGCTGGT
CATCCTGGCCTGTGTGCTGTGGAAGAAGCGGATCAAGCCCATCGTGTGGCCCAGCCT
GCCTGACCACAAGAAAACCCTGGAACACCTGTGCAAGAAGCCCCGGAAGAACCTGA
ACGTGTCCTTCAATCCCGAGAGCTTCCTGGACTGTCAGATCCACAGAGTGGACGACA
TCCAGGCCAGGGACGAAGTGGAAGGCTTTCTGCAGGACACATTCCCTCAGCAGCTG
GAAGAGAGCGAGAAGCAGAGACTTGGAGGCGACGTGCAGAGCCCTAATTGCCCTTC
TGAGGACGTGGTCATCACCCCAGAGAGCTTCGGCAGAGATAGCAGCCTGACATGTC
TGGCCGGCAATGTGTCCGCCTGTGATGCCCCTATCCTGAGCAGCAGCAGAAGCCTGG
ATTGCAGAGAGAGCGGCAAGAACGGCCCTCACGTGTACCAGGATCTGCTCCTGTCT
CTGGGCACCACCAATAGCACACTGCCTCCACCATTCAGCCTGCAGAGCGGCATCCTG
ACACTGAACCCTGTTGCTCAGGGCCAGCCAATCCTGACAAGCCTGGGCAGCAATCA
AGAAGAGGCCTACGTCACCATGAGCAGCTTCTACCAGAATCAAG
SEQ ID NO: 54 IL-21R extracellular(hinge)
SDPVIFQTQSEELKE
SEQ ID NO: 55 IL-21R extracellular(hinge)
AGCGACCCCGTGATCTTTCAGACCCAGAGCGAGGAACTGAAAGAA
SEQ ID NO: 56 IL-21R transmembrane
GWNPHLLLLLLLVIVFIPAFW
SEQ ID NO: 57 IL-21R transmembrane
GGCTGGAACCCTCACCTCCTGCTGTTGCTGCTGCTGGTCATCGTGTTCATCCCCGCCT
TTTGG
SEQ ID NO: 58 IL-21R intracellular
SLKTHPLWRLWKKIWAVPSPERFFMPLYKGCSGDFKKWVGAPFTGSSLELGPWSPEVPS
TLEVYSCHPPRSPAKRLQLTELQEPAELVESDGVPKPSFWPTAQNSGGSAYSEERDRPYG
LVSIDTVTVLDAEGPCTWPCSCEDDGYPALDLDAGLEPSPGLEDPLLDAGTTVLSCGCV
SAGSPGLGGPLGSLLDRLKPPLADGEDWAGGLPWGGRSPGGVSESEAGSPLAGLDMDT
FDSGFVGSDCSSPVECDFTSPGDEGPPRSYLRQWVVIPPPLSSPGPQAS
SEQ ID NO: 59 IL-21R intracellular
AGCCTGAAAACACACCCTCTGTGGCGGCTGTGGAAGAAAATCTGGGCCGTGCCATC
TCCTGAGCGGTTCTTCATGCCTCTGTACAAGGGCTGCAGCGGCGACTTCAAGAAATG
GGTCGGAGCCCCTTTTACCGGCAGCTCTCTGGAACTTGGACCTTGGAGCCCTGAGGT
GCCCAGCACACTGGAAGTGTACAGCTGTCACCCTCCTAGAAGCCCCGCCAAGAGAC
TGCAGCTGACAGAGCTGCAAGAGCCTGCCGAGCTGGTGGAATCTGATGGCGTGCCC
AAGCCTAGCTTCTGGCCCACAGCTCAGAATAGCGGCGGCTCTGCCTACAGCGAGGA
AAGAGATAGACCCTACGGCCTGGTGTCCATCGACACCGTGACAGTGCTGGATGCCG
AGGGACCTTGTACCTGGCCTTGTAGCTGCGAGGACGATGGCTACCCTGCTCTGGATC
TGGACGCTGGCCTTGAGCCTTCTCCAGGACTGGAAGATCCTCTGCTGGACGCCGGAA
CAACCGTGCTGTCTTGTGGCTGTGTGTCTGCCGGATCTCCTGGACTTGGAGGCCCTCT
GGGAAGCCTGCTGGATAGACTGAAACCTCCTCTGGCCGACGGCGAAGATTGGGCTG
GTGGACTTCCTTGGGGCGGAAGATCTCCAGGCGGAGTGTCTGAGTCTGAAGCCGGTT
CTCCACTGGCCGGCCTGGACATGGATACCTTCGATTCTGGCTTCGTGGGCAGCGACT
GTAGCAGCCCTGTGGAATGCGACTTCACAAGCCCTGGCGACGAGGGCCCACCTAGA
AGCTATCTGAGACAGTGGGTCGTGATCCCTCCACCTCTGTCTAGTCCTGGACCTCAG
GCTTCT
SEQ ID NO: 60 IL-21R (extracellular, transmembrane, intracellular)
SDPVIFQTQSEELKEGWNPHLLLLLLLVIVFIPAFWSLKTHPLWRLWKKIWAVPSPERFF
MPLYKGCSGDFKKWVGAPFTGSSLELGPWSPEVPSTLEVYSCHPPRSPAKRLQLTELQE
PAELVESDGVPKPSFWPTAQNSGGSAYSEERDRPYGLVSIDTVTVLDAEGPCTWPCSCE
DDGYPALDLDAGLEPSPGLEDPLLDAGTTVLSCGCVSAGSPGLGGPLGSLLDRLKPPLA
DGEDWAGGLPWGGRSPGGVSESEAGSPLAGLDMDTFDSGFVGSDCSSPVECDFTSPGD
EGPPRSYLRQWVVIPPPLSSPGPQAS
SEQ ID NO: 61 IL-21R (extracellular, transmembrane, intracellular)
AGCGACCCCGTGATCTTTCAGACCCAGAGCGAGGAACTGAAAGAAGGCTGGAACCC
TCACCTCCTGCTGTTGCTGCTGCTGGTCATCGTGTTCATCCCCGCCTTTTGGAGCCTG
AAAACACACCCTCTGTGGCGGCTGTGGAAGAAAATCTGGGCCGTGCCATCTCCTGA
GCGGTTCTTCATGCCTCTGTACAAGGGCTGCAGCGGCGACTTCAAGAAATGGGTCGG
AGCCCCTTTTACCGGCAGCTCTCTGGAACTTGGACCTTGGAGCCCTGAGGTGCCCAG
CACACTGGAAGTGTACAGCTGTCACCCTCCTAGAAGCCCCGCCAAGAGACTGCAGC
TGACAGAGCTGCAAGAGCCTGCCGAGCTGGTGGAATCTGATGGCGTGCCCAAGCCT
AGCTTCTGGCCCACAGCTCAGAATAGCGGCGGCTCTGCCTACAGCGAGGAAAGAGA
TAGACCCTACGGCCTGGTGTCCATCGACACCGTGACAGTGCTGGATGCCGAGGGAC
CTTGTACCTGGCCTTGTAGCTGCGAGGACGATGGCTACCCTGCTCTGGATCTGGACG
CTGGCCTTGAGCCTTCTCCAGGACTGGAAGATCCTCTGCTGGACGCCGGAACAACCG
TGCTGTCTTGTGGCTGTGTGTCTGCCGGATCTCCTGGACTTGGAGGCCCTCTGGGAA
GCCTGCTGGATAGACTGAAACCTCCTCTGGCCGACGGCGAAGATTGGGCTGGTGGA
CTTCCTTGGGGCGGAAGATCTCCAGGCGGAGTGTCTGAGTCTGAAGCCGGTTCTCCA
CTGGCCGGCCTGGACATGGATACCTTCGATTCTGGCTTCGTGGGCAGCGACTGTAGC
AGCCCTGTGGAATGCGACTTCACAAGCCCTGGCGACGAGGGCCCACCTAGAAGCTA
TCTGAGACAGTGGGTCGTGATCCCTCCACCTCTGTCTAGTCCTGGACCTCAGGCTTCT
SEQ ID NO: 62 ZipIL-21R: EE2Xzip.IL21R.2A.RR2Xzip.IL2Rg.2A.mClover_IL21R chain
MDPDLEIEAAFLERENTALETRVAELRQRVQRLRNRVSQYRTRYGPLGGGKGGGGSGG
GGSGGGGSMDPDLEIEAAFLERENTALETRVAELRQRVQRLRNRVSQYRTRYGPLGGG
KSDPVIFQTQSEELKEGWNPHLLLLLLLVIVFIPAFWSLKTHPLWRLWKKIWAVPSPERF
FMPLYKGCSGDFKKWVGAPFTGSSLELGPWSPEVPSTLEVYSCHPPRSPAKRLQLTELQ
EPAELVESDGVPKPSFWPTAQNSGGSAYSEERDRPYGLVSIDTVTVLDAEGPCTWPCSC
EDDGYPALDLDAGLEPSPGLEDPLLDAGTTVLSCGCVSAGSPGLGGPLGSLLDRLKPPL
ADGEDWAGGLPWGGRSPGGVSESEAGSPLAGLDMDTFDSGFVGSDCSSPVECDFTSPG
DEGPPRSYLRQWVVIPPPLSSPGPQAS
SEQ ID NO: 63 ZipIL-21R: EE2Xzip.IL21R.2A.RR2Xzip.IL2Rg.2A.mClover_IL21R chain
ATGGACCCCGACCTGGAAATCGAGGCCGCCTTCCTGGAAAGAGAGAACACAGCCCT
GGAAACCAGAGTGGCCGAGCTGAGACAGAGAGTGCAGCGGCTGAGAAACCGGGTG
TCCCAGTACAGAACCAGATACGGACCTCTCGGCGGAGGAAAAGGTGGCGGAGGTTC
TGGCGGAGGCGGCTCTGGTGGTGGTGGATCTATGGATCCTGACCTCGAGATTGAAGC
CGCCTTTCTCGAGCGCGAGAATACCGCTCTCGAGACAAGAGTGGCTGAACTGCGGC
AGAGGGTCCAGAGACTGAGGAACAGAGTGTCTCAGTACCGGACACGCTACGGCCCT
CTTGGAGGTGGAAAAAGCGACCCCGTGATCTTTCAGACCCAGAGCGAGGAACTGAA
AGAAGGCTGGAACCCTCACCTCCTGCTGTTGCTGCTGCTGGTCATCGTGTTCATCCC
CGCCTTTTGGAGCCTGAAAACACACCCTCTGTGGCGGCTGTGGAAGAAAATCTGGGC
CGTGCCATCTCCTGAGCGGTTCTTCATGCCTCTGTACAAGGGCTGCAGCGGCGACTT
CAAGAAATGGGTCGGAGCCCCTTTTACCGGCAGCTCTCTGGAACTTGGACCTTGGAG
CCCTGAGGTGCCCAGCACACTGGAAGTGTACAGCTGTCACCCTCCTAGAAGCCCCGC
CAAGAGACTGCAGCTGACAGAGCTGCAAGAGCCTGCCGAGCTGGTGGAATCTGATG
GCGTGCCCAAGCCTAGCTTCTGGCCCACAGCTCAGAATAGCGGCGGCTCTGCCTACA
GCGAGGAAAGAGATAGACCCTACGGCCTGGTGTCCATCGACACCGTGACAGTGCTG
GATGCCGAGGGACCTTGTACCTGGCCTTGTAGCTGCGAGGACGATGGCTACCCTGCT
CTGGATCTGGACGCTGGCCTTGAGCCTTCTCCAGGACTGGAAGATCCTCTGCTGGAC
GCCGGAACAACCGTGCTGTCTTGTGGCTGTGTGTCTGCCGGATCTCCTGGACTTGGA
GGCCCTCTGGGAAGCCTGCTGGATAGACTGAAACCTCCTCTGGCCGACGGCGAAGA
TTGGGCTGGTGGACTTCCTTGGGGCGGAAGATCTCCAGGCGGAGTGTCTGAGTCTGA
AGCCGGTTCTCCACTGGCCGGCCTGGACATGGATACCTTCGATTCTGGCTTCGTGGG
CAGCGACTGTAGCAGCCCTGTGGAATGCGACTTCACAAGCCCTGGCGACGAGGGCC
CACCTAGAAGCTATCTGAGACAGTGGGTCGTGATCCCTCCACCTCTGTCTAGTCCTG
GACCTCAGGCTTCT
SEQ ID NO: 64 IL9R extracellular(hinge)
APQRQGPLIPPWGWP
SEQ ID NO: 65 IL9R extracellular(hinge)
GCTCCTCAGAGACAGGGACCTCTGATTCCTCCTTGGGGCTGGCCT
SEQ ID NO: 66 IL9R transmembrane
GNTLVAVSIFLLLTGPTYLLF
SEQ ID NO: 67 IL9R transmembrane
GGAAATACCCTGGTGGCCGTGTCCATCTTCCTGCTGCTGACCGGACCAACCTACCTG
CTGTTC
SEQ ID NO: 68 IL9R intracellular
KLSPRVKRIFYQNVPSPAMFFQPLYSVHNGNFQTWMGAHGAGVLLSQDCAGTPQGALE
PCVQEATALLTCGPARPWKSVALEEEQEGPGTRLPGNLSSEDVLPAGCTEWRVQTLAYL
PQEDWAPTSLTRPAPPDSEGSRSSSSSSSSNNNNYCALGCYGGWHLSALPGNTQSSGPIP
ALACGLSCDHQGLETQQGVAWVLAGHCQRPGLHEDLQGMLLPSVLSKARSWTF
SEQ ID NO: 69 IL9R intracellular
AAGCTGAGCCCCAGAGTGAAGCGGATCTTCTACCAGAACGTGCCCTCTCCTGCCATG
TTCTTCCAGCCTCTGTACAGCGTGCACAACGGCAACTTCCAGACATGGATGGGAGCA
CATGGTGCCGGCGTGCTGCTGTCTCAAGATTGTGCTGGAACACCCCAGGGCGCTCTG
GAACCTTGTGTGCAAGAAGCCACCGCTCTGCTGACATGCGGACCTGCCAGACCTTGG
AAAAGCGTGGCCCTGGAAGAGGAACAAGAAGGCCCTGGCACAAGACTGCCCGGCA
ATCTGTCTAGCGAGGATGTGCTGCCTGCCGGCTGTACTGAATGGCGAGTTCAGACCC
TGGCCTACCTGCCTCAAGAAGATTGGGCCCCTACCAGCCTGACCAGACCTGCTCCTC
CTGATAGCGAGGGCAGCAGAAGCAGCAGCAGCTCCAGCTCCTCCAACAACAACAAT
TACTGTGCCCTGGGCTGCTACGGCGGCTGGCATCTTTCTGCTCTGCCTGGCAACACA
CAGAGCAGCGGCCCTATTCCTGCTCTGGCCTGTGGCCTGAGCTGTGATCACCAGGGA
CTCGAGACACAGCAGGGCGTTGCATGGGTTCTCGCCGGCCATTGTCAAAGACCTGG
CCTGCACGAAGATCTGCAGGGCATGCTGCTGCCTAGCGTGCTGTCTAAGGCCAGAA
GCTGGACCTTC
SEQ ID NO: 70 IL9R (extracellular, transmembrane, intracellular)
APQRQGPLIPPWGWPGNTLVAVSIFLLLTGPTYLLFKLSPRVKRIFYQNVPSPAMFFQPL
YSVHNGNFQTWMGAHGAGVLLSQDCAGTPQGALEPCVQEATALLTCGPARPWKSVAL
EEEQEGPGTRLPGNLSSEDVLPAGCTEWRVQTLAYLPQEDWAPTSLTRPAPPDSEGSRSS
SSSSSSNNNNYCALGCYGGWHLSALPGNTQSSGPIPALACGLSCDHQGLETQQGVAWV
LAGHCQRPGLHEDLQGMLLPSVLSKARSWTF
SEQ ID NO: 71 IL9R(extracellular, transmembrane, intracellular)
GCTCCTCAGAGACAGGGACCTCTGATTCCTCCTTGGGGCTGGCCTGGAAATACCCTG
GTGGCCGTGTCCATCTTCCTGCTGCTGACCGGACCAACCTACCTGCTGTTCAAGCTG
AGCCCCAGAGTGAAGCGGATCTTCTACCAGAACGTGCCCTCTCCTGCCATGTTCTTC
CAGCCTCTGTACAGCGTGCACAACGGCAACTTCCAGACATGGATGGGAGCACATGG
TGCCGGCGTGCTGCTGTCTCAAGATTGTGCTGGAACACCCCAGGGCGCTCTGGAACC
TTGTGTGCAAGAAGCCACCGCTCTGCTGACATGCGGACCTGCCAGACCTTGGAAAA
GCGTGGCCCTGGAAGAGGAACAAGAAGGCCCTGGCACAAGACTGCCCGGCAATCTG
TCTAGCGAGGATGTGCTGCCTGCCGGCTGTACTGAATGGCGAGTTCAGACCCTGGCC
TACCTGCCTCAAGAAGATTGGGCCCCTACCAGCCTGACCAGACCTGCTCCTCCTGAT
AGCGAGGGCAGCAGAAGCAGCAGCAGCTCCAGCTCCTCCAACAACAACAATTACTG
TGCCCTGGGCTGCTACGGCGGCTGGCATCTTTCTGCTCTGCCTGGCAACACACAGAG
CAGCGGCCCTATTCCTGCTCTGGCCTGTGGCCTGAGCTGTGATCACCAGGGACTCGA
GACACAGCAGGGCGTTGCATGGGTTCTCGCCGGCCATTGTCAAAGACCTGGCCTGC
ACGAAGATCTGCAGGGCATGCTGCTGCCTAGCGTGCTGTCTAAGGCCAGAAGCTGG
ACCTTC
SEQ ID NO: 72 ZipIL9R: EE2Xzip.IL9R.2A.RR2Xzip.IL2Rg.2A.mClover_IL9R chain
MDPDLEIEAAFLERENTALETRVAELRQRVQRLRNRVSQYRTRYGPLGGGKGGGGSGG
GGSGGGGSMDPDLEIEAAFLERENTALETRVAELRQRVQRLRNRVSQYRTRYGPLGGG
KAPQRQGPLIPPWGWPGNTLVAVSIFLLLTGPTYLLFKLSPRVKRIFYQNVPSPAMFFQP
LYSVHNGNFQTWMGAHGAGVLLSQDCAGTPQGALEPCVQEATALLTCGPARPWKSVA
LEEEQEGPGTRLPGNLSSEDVLPAGCTEWRVQTLAYLPQEDWAPTSLTRPAPPDSEGSRS
SSSSSSSNNNNYCALGCYGGWHLSALPGNTQSSGPIPALACGLSCDHQGLETQQGVAW
VLAGHCQRPGLHEDLQGMLLPSVLSKARSWTF
SEQ ID NO: 73 ZipIL9R: EE2Xzip.IL9R.2A.RR2Xzip.IL2Rg.2A.mClover_IL9R chain
ATGGACCCCGACCTGGAAATCGAGGCCGCCTTCCTGGAAAGAGAGAACACAGCCCT
GGAAACCAGAGTGGCCGAGCTGAGACAGAGAGTGCAGCGGCTGAGAAACCGGGTG
TCCCAGTACAGAACCAGATACGGACCTCTCGGCGGAGGAAAAGGTGGCGGAGGTTC
TGGCGGAGGCGGCTCTGGTGGTGGTGGATCTATGGATCCTGACCTCGAGATTGAAGC
CGCCTTTCTCGAGCGCGAGAATACCGCTCTCGAGACAAGAGTGGCTGAACTGCGGC
AGAGGGTCCAGAGACTGAGGAACAGAGTGTCTCAGTACCGGACACGCTACGGCCCT
CTTGGAGGTGGAAAAGCTCCTCAGAGACAGGGACCTCTGATTCCTCCTTGGGGCTGG
CCTGGAAATACCCTGGTGGCCGTGTCCATCTTCCTGCTGCTGACCGGACCAACCTAC
CTGCTGTTCAAGCTGAGCCCCAGAGTGAAGCGGATCTTCTACCAGAACGTGCCCTCT
CCTGCCATGTTCTTCCAGCCTCTGTACAGCGTGCACAACGGCAACTTCCAGACATGG
ATGGGAGCACATGGTGCCGGCGTGCTGCTGTCTCAAGATTGTGCTGGAACACCCCAG
GGCGCTCTGGAACCTTGTGTGCAAGAAGCCACCGCTCTGCTGACATGCGGACCTGCC
AGACCTTGGAAAAGCGTGGCCCTGGAAGAGGAACAAGAAGGCCCTGGCACAAGAC
TGCCCGGCAATCTGTCTAGCGAGGATGTGCTGCCTGCCGGCTGTACTGAATGGCGAG
TTCAGACCCTGGCCTACCTGCCTCAAGAAGATTGGGCCCCTACCAGCCTGACCAGAC
CTGCTCCTCCTGATAGCGAGGGCAGCAGAAGCAGCAGCAGCTCCAGCTCCTCCAAC
AACAACAATTACTGTGCCCTGGGCTGCTACGGCGGCTGGCATCTTTCTGCTCTGCCT
GGCAACACACAGAGCAGCGGCCCTATTCCTGCTCTGGCCTGTGGCCTGAGCTGTGAT
CACCAGGGACTCGAGACACAGCAGGGCGTTGCATGGGTTCTCGCCGGCCATTGTCA
AAGACCTGGCCTGCACGAAGATCTGCAGGGCATGCTGCTGCCTAGCGTGCTGTCTAA
GGCCAGAAGCTGGACCTTC
SEQ ID NO: 74 IL4R alpha extracellular(hinge)
STKWHNSYREPFEQH
SEQ ID NO: 75 IL4R alpha extracellular(hinge)
AGCACCAAGTGGCACAACAGCTACAGGGAGCCCTTCGAGCAGCAC
SEQ ID NO: 76 IL4R alpha transmembrane
LLLGVSVSCIVILAVCLLCYVSIT
SEQ ID NO: 77 IL4R alpha transmembrane
CTGCTGCTGGGCGTGAGCGTGAGCTGCATCGTGATCCTGGCCGTGTGCCTGCTGTGC
TACGTGAGCATCACC
SEQ ID NO: 78 IL4R alpha intracellular
KIKKEWWDQIPNPARSRLVAIIIQDAQGSQWEKRSRGQEPAKCPHWKNCLTKLLPCFLE
HNMKRDEDPHKAAKEMPFQGSGKSAWCPVEISKTVLWPESISVVRCVELFEAPVECEEE
EEVEEEKGSFCASPESSRDDFQEGREGIVARLTESLFLDLLGEENGGFCQQDMGESCLLP
PSGSTSAHMPWDEFPSAGPKEAPPWGKEQPLHLEPSPPASPTQSPDNLTCTETPLVIAGN
PAYRSFSNSLSQSPCPRELGPDPLLARHLEEVEPEMPCVPQLSEPTTVPQPEPETWEQILR
RNVLQHGAAAAPVSAPTSGYQEFVHAVEQGGTQASAVVGLGPPGEAGYKAFSSLLASS
AVSPEKCGFGASSGEEGYKPFQDLIPGCPGDPAPVPVPLFTFGLDREPPRSPQSSHLPSSSP
EHLGLEPGEKVEDMPKPPLPQEQATDPLVDSLGSGIVYSALTCHLCGHLKQCHGQEDGG
QTPVMASPCCGCCCGDRSSPPTTPLRAPDPSPGGVPLEASLCPASLAPSGISEKSKSSSSFH
PAPGNAQSSSQTPKIVNFVSVGPTYMRVS
SEQ ID NO: 79 IL4R alpha intracellular
AAGATCAAGAAGGAGTGGTGGGACCAGATCCCCAACCCCGCCAGGAGCAGGCTGGT
GGCCATCATCATCCAGGACGCCCAGGGCAGCCAGTGGGAGAAGAGGAGCAGGGGC
CAGGAGCCCGCCAAGTGCCCCCACTGGAAGAACTGCCTGACCAAGCTGCTGCCCTG
CTTCCTGGAGCACAACATGAAGAGGGACGAGGACCCCCACAAGGCCGCCAAGGAG
ATGCCCTTCCAGGGCAGCGGCAAGAGCGCCTGGTGCCCCGTGGAGATCAGCAAGAC
CGTGCTGTGGCCCGAGAGCATCAGCGTGGTGAGGTGCGTGGAGCTGTTCGAGGCCC
CCGTGGAGTGCGAGGAGGAGGAGGAGGTGGAGGAGGAGAAGGGCAGCTTCTGCGC
CAGCCCCGAGAGCAGCAGGGACGACTTCCAGGAGGGCAGGGAGGGCATCGTGGCC
AGGCTGACCGAGAGCCTGTTCCTGGACCTGCTGGGCGAGGAGAACGGCGGCTTCTG
CCAGCAGGACATGGGCGAGAGCTGCCTGCTGCCCCCCAGCGGCAGCACCAGCGCCC
ACATGCCCTGGGACGAGTTCCCCAGCGCCGGCCCCAAGGAGGCCCCCCCCTGGGGC
AAGGAGCAGCCCCTGCACCTGGAGCCCAGCCCCCCCGCCAGCCCCACCCAGAGCCC
CGACAACCTGACCTGCACCGAGACCCCCCTGGTGATCGCCGGCAACCCCGCCTACA
GGAGCTTCAGCAACAGCCTGAGCCAGAGCCCCTGCCCCAGGGAGCTGGGCCCCGAC
CCCCTGCTGGCCAGGCACCTGGAGGAGGTGGAGCCCGAGATGCCCTGCGTGCCCCA
GCTGAGCGAGCCCACCACCGTGCCCCAGCCCGAGCCCGAGACCTGGGAGCAGATCC
TGAGGAGGAACGTGCTGCAGCACGGCGCCGCCGCCGCCCCCGTGAGCGCCCCCACC
AGCGGCTACCAGGAGTTCGTGCACGCCGTGGAGCAGGGCGGCACCCAGGCCAGCGC
CGTGGTGGGCCTGGGCCCCCCCGGCGAGGCCGGCTACAAGGCCTTCAGCAGCCTGC
TGGCCAGCAGCGCCGTGAGCCCCGAGAAGTGCGGCTTCGGCGCCAGCAGCGGCGAG
GAGGGCTACAAGCCCTTCCAGGACCTGATCCCCGGCTGCCCCGGCGACCCCGCCCCC
GTGCCCGTGCCCCTGTTCACCTTCGGCCTGGACAGGGAGCCCCCCAGGAGCCCCCAG
AGCAGCCACCTGCCCAGCAGCAGCCCCGAGCACCTGGGCCTGGAGCCCGGCGAGAA
GGTGGAGGACATGCCCAAGCCCCCCCTGCCCCAGGAGCAGGCCACCGACCCCCTGG
TGGACAGCCTGGGCAGCGGCATCGTGTACAGCGCCCTGACCTGCCACCTGTGCGGC
CACCTGAAGCAGTGCCACGGCCAGGAGGACGGCGGCCAGACCCCCGTGATGGCCAG
CCCCTGCTGCGGCTGCTGCTGCGGCGACAGGAGCAGCCCCCCCACCACCCCCCTGAG
GGCCCCCGACCCCAGCCCCGGCGGCGTGCCCCTGGAGGCCAGCCTGTGCCCCGCCA
GCCTGGCCCCCAGCGGCATCAGCGAGAAGAGCAAGAGCAGCAGCAGCTTCCACCCC
GCCCCCGGCAACGCCCAGAGCAGCAGCCAGACCCCCAAGATCGTGAACTTCGTGAG
CGTGGGCCCCACCTACATGAGGGTGAGC
SEQ ID NO: 80 IL4R alpha (extracellular, transmembrane, intracellular)
STKWHNSYREPFEQHLLLGVSVSCIVILAVCLLCYVSITKIKKEWWDQIPNPARSRLVAIII
QDAQGSQWEKRSRGQEPAKCPHWKNCLTKLLPCFLEHNMKRDEDPHKAAKEMPFQGS
GKSAWCPVEISKTVLWPESISVVRCVELFEAPVECEEEEEVEEEKGSFCASPESSRDDFQE
GREGIVARLTESLFLDLLGEENGGFCQQDMGESCLLPPSGSTSAHMPWDEFPSAGPKEA
PPWGKEQPLHLEPSPPASPTQSPDNLTCTETPLVIAGNPAYRSFSNSLSQSPCPRELGPDPL
LARHLEEVEPEMPCVPQLSEPTTVPQPEPETWEQILRRNVLQHGAAAAPVSAPTSGYQEF
VHAVEQGGTQASAVVGLGPPGEAGYKAFSSLLASSAVSPEKCGFGASSGEEGYKPFQDL
IPGCPGDPAPVPVPLFTFGLDREPPRSPQSSHLPSSSPEHLGLEPGEKVEDMPKPPLPQEQA
TDPLVDSLGSGIVYSALTCHLCGHLKQCHGQEDGGQTPVMASPCCGCCCGDRSSPPTTP
LRAPDPSPGGVPLEASLCPASLAPSGISEKSKSSSSFHPAPGNAQSSSQTPKIVNFVSVGPT
YMRVS
SEQ ID NO: 81 IL9R alpha(extracellular, transmembrane, intracellular)
AGCACCAAGTGGCACAACAGCTACAGGGAGCCCTTCGAGCAGCACCTGCTGCTGGG
CGTGAGCGTGAGCTGCATCGTGATCCTGGCCGTGTGCCTGCTGTGCTACGTGAGCAT
CACCAAGATCAAGAAGGAGTGGTGGGACCAGATCCCCAACCCCGCCAGGAGCAGG
CTGGTGGCCATCATCATCCAGGACGCCCAGGGCAGCCAGTGGGAGAAGAGGAGCAG
GGGCCAGGAGCCCGCCAAGTGCCCCCACTGGAAGAACTGCCTGACCAAGCTGCTGC
CCTGCTTCCTGGAGCACAACATGAAGAGGGACGAGGACCCCCACAAGGCCGCCAAG
GAGATGCCCTTCCAGGGCAGCGGCAAGAGCGCCTGGTGCCCCGTGGAGATCAGCAA
GACCGTGCTGTGGCCCGAGAGCATCAGCGTGGTGAGGTGCGTGGAGCTGTTCGAGG
CCCCCGTGGAGTGCGAGGAGGAGGAGGAGGTGGAGGAGGAGAAGGGCAGCTTCTG
CGCCAGCCCCGAGAGCAGCAGGGACGACTTCCAGGAGGGCAGGGAGGGCATCGTG
GCCAGGCTGACCGAGAGCCTGTTCCTGGACCTGCTGGGCGAGGAGAACGGCGGCTT
CTGCCAGCAGGACATGGGCGAGAGCTGCCTGCTGCCCCCCAGCGGCAGCACCAGCG
CCCACATGCCCTGGGACGAGTTCCCCAGCGCCGGCCCCAAGGAGGCCCCCCCCTGG
GGCAAGGAGCAGCCCCTGCACCTGGAGCCCAGCCCCCCCGCCAGCCCCACCCAGAG
CCCCGACAACCTGACCTGCACCGAGACCCCCCTGGTGATCGCCGGCAACCCCGCCTA
CAGGAGCTTCAGCAACAGCCTGAGCCAGAGCCCCTGCCCCAGGGAGCTGGGCCCCG
ACCCCCTGCTGGCCAGGCACCTGGAGGAGGTGGAGCCCGAGATGCCCTGCGTGCCC
CAGCTGAGCGAGCCCACCACCGTGCCCCAGCCCGAGCCCGAGACCTGGGAGCAGAT
CCTGAGGAGGAACGTGCTGCAGCACGGCGCCGCCGCCGCCCCCGTGAGCGCCCCCA
CCAGCGGCTACCAGGAGTTCGTGCACGCCGTGGAGCAGGGCGGCACCCAGGCCAGC
GCCGTGGTGGGCCTGGGCCCCCCCGGCGAGGCCGGCTACAAGGCCTTCAGCAGCCT
GCTGGCCAGCAGCGCCGTGAGCCCCGAGAAGTGCGGCTTCGGCGCCAGCAGCGGCG
AGGAGGGCTACAAGCCCTTCCAGGACCTGATCCCCGGCTGCCCCGGCGACCCCGCC
CCCGTGCCCGTGCCCCTGTTCACCTTCGGCCTGGACAGGGAGCCCCCCAGGAGCCCC
CAGAGCAGCCACCTGCCCAGCAGCAGCCCCGAGCACCTGGGCCTGGAGCCCGGCGA
GAAGGTGGAGGACATGCCCAAGCCCCCCCTGCCCCAGGAGCAGGCCACCGACCCCC
TGGTGGACAGCCTGGGCAGCGGCATCGTGTACAGCGCCCTGACCTGCCACCTGTGC
GGCCACCTGAAGCAGTGCCACGGCCAGGAGGACGGCGGCCAGACCCCCGTGATGGC
CAGCCCCTGCTGCGGCTGCTGCTGCGGCGACAGGAGCAGCCCCCCCACCACCCCCCT
GAGGGCCCCCGACCCCAGCCCCGGCGGCGTGCCCCTGGAGGCCAGCCTGTGCCCCG
CCAGCCTGGCCCCCAGCGGCATCAGCGAGAAGAGCAAGAGCAGCAGCAGCTTCCAC
CCCGCCCCCGGCAACGCCCAGAGCAGCAGCCAGACCCCCAAGATCGTGAACTTCGT
GAGCGTGGGCCCCACCTACATGAGGGTGAGC
SEQ ID NO: 82 ZipIL4R: EE2Xzip.IL4Ra.2A.RR2Xzip.IL2Rg.2A.mClover_Il4Ra
MDPDLEIEAAFLERENTALETRVAELRQRVQRLRNRVSQYRTRYGPLGGGKGGGGSGG
GGSGGGGSMDPDLEIEAAFLERENTALETRVAELRQRVQRLRNRVSQYRTRYGPLGGG
KSTKWHNSYREPFEQHLLLGVSVSCIVILAVCLLCYVSITKIKKEWWDQIPNPARSRLVA
IIIQDAQGSQWEKRSRGQEPAKCPHWKNCLTKLLPCFLEHNMKRDEDPHKAAKEMPFQ
GSGKSAWCPVEISKTVLWPESISVVRCVELFEAPVECEEEEEVEEEKGSFCASPESSRDDF
QEGREGIVARLTESLFLDLLGEENGGFCQQDMGESCLLPPSGSTSAHMPWDEFPSAGPK
EAPPWGKEQPLHLEPSPPASPTQSPDNLTCTETPLVIAGNPAYRSFSNSLSQSPCPRELGP
DPLLARHLEEVEPEMPCVPQLSEPTTVPQPEPETWEQILRRNVLQHGAAAAPVSAPTSGY
QEFVHAVEQGGTQASAVVGLGPPGEAGYKAFSSLLASSAVSPEKCGFGASSGEEGYKPF
QDLIPGCPGDPAPVPVPLFTFGLDREPPRSPQSSHLPSSSPEHLGLEPGEKVEDMPKPPLPQ
EQATDPLVDSLGSGIVYSALTCHLCGHLKQCHGQEDGGQTPVMASPCCGCCCGDRSSPP
TTPLRAPDPSPGGVPLEASLCPASLAPSGISEKSKSSSSFHPAPGNAQSSSQTPKIVNFVSV
GPTYMRVS
SEQ ID NO: 83 ZipIL4R: EE2Xzip.IL4Ra.2A.RR2Xzip.IL2Rg.2A.mClover Il4Ra
ATGGACCCCGACCTGGAAATCGAGGCCGCCTTCCTGGAAAGAGAGAACACAGCCCT
GGAAACCAGAGTGGCCGAGCTGAGACAGAGAGTGCAGCGGCTGAGAAACCGGGTG
TCCCAGTACAGAACCAGATACGGACCTCTCGGCGGAGGAAAAGGTGGCGGAGGTTC
TGGCGGAGGCGGCTCTGGTGGTGGTGGATCTATGGATCCTGACCTCGAGATTGAAGC
CGCCTTTCTCGAGCGCGAGAATACCGCTCTCGAGACAAGAGTGGCTGAACTGCGGC
AGAGGGTCCAGAGACTGAGGAACAGAGTGTCTCAGTACCGGACACGCTACGGCCCT
CTTGGAGGTGGAAAAAGCACCAAGTGGCACAACAGCTACAGGGAGCCCTTCGAGCA
GCACCTGCTGCTGGGCGTGAGCGTGAGCTGCATCGTGATCCTGGCCGTGTGCCTGCT
GTGCTACGTGAGCATCACCAAGATCAAGAAGGAGTGGTGGGACCAGATCCCCAACC
CCGCCAGGAGCAGGCTGGTGGCCATCATCATCCAGGACGCCCAGGGCAGCCAGTGG
GAGAAGAGGAGCAGGGGCCAGGAGCCCGCCAAGTGCCCCCACTGGAAGAACTGCC
TGACCAAGCTGCTGCCCTGCTTCCTGGAGCACAACATGAAGAGGGACGAGGACCCC
CACAAGGCCGCCAAGGAGATGCCCTTCCAGGGCAGCGGCAAGAGCGCCTGGTGCCC
CGTGGAGATCAGCAAGACCGTGCTGTGGCCCGAGAGCATCAGCGTGGTGAGGTGCG
TGGAGCTGTTCGAGGCCCCCGTGGAGTGCGAGGAGGAGGAGGAGGTGGAGGAGGA
GAAGGGCAGCTTCTGCGCCAGCCCCGAGAGCAGCAGGGACGACTTCCAGGAGGGCA
GGGAGGGCATCGTGGCCAGGCTGACCGAGAGCCTGTTCCTGGACCTGCTGGGCGAG
GAGAACGGCGGCTTCTGCCAGCAGGACATGGGCGAGAGCTGCCTGCTGCCCCCCAG
CGGCAGCACCAGCGCCCACATGCCCTGGGACGAGTTCCCCAGCGCCGGCCCCAAGG
AGGCCCCCCCCTGGGGCAAGGAGCAGCCCCTGCACCTGGAGCCCAGCCCCCCCGCC
AGCCCCACCCAGAGCCCCGACAACCTGACCTGCACCGAGACCCCCCTGGTGATCGC
CGGCAACCCCGCCTACAGGAGCTTCAGCAACAGCCTGAGCCAGAGCCCCTGCCCCA
GGGAGCTGGGCCCCGACCCCCTGCTGGCCAGGCACCTGGAGGAGGTGGAGCCCGAG
ATGCCCTGCGTGCCCCAGCTGAGCGAGCCCACCACCGTGCCCCAGCCCGAGCCCGA
GACCTGGGAGCAGATCCTGAGGAGGAACGTGCTGCAGCACGGCGCCGCCGCCGCCC
CCGTGAGCGCCCCCACCAGCGGCTACCAGGAGTTCGTGCACGCCGTGGAGCAGGGC
GGCACCCAGGCCAGCGCCGTGGTGGGCCTGGGCCCCCCCGGCGAGGCCGGCTACAA
GGCCTTCAGCAGCCTGCTGGCCAGCAGCGCCGTGAGCCCCGAGAAGTGCGGCTTCG
GCGCCAGCAGCGGCGAGGAGGGCTACAAGCCCTTCCAGGACCTGATCCCCGGCTGC
CCCGGCGACCCCGCCCCCGTGCCCGTGCCCCTGTTCACCTTCGGCCTGGACAGGGAG
CCCCCCAGGAGCCCCCAGAGCAGCCACCTGCCCAGCAGCAGCCCCGAGCACCTGGG
CCTGGAGCCCGGCGAGAAGGTGGAGGACATGCCCAAGCCCCCCCTGCCCCAGGAGC
AGGCCACCGACCCCCTGGTGGACAGCCTGGGCAGCGGCATCGTGTACAGCGCCCTG
ACCTGCCACCTGTGCGGCCACCTGAAGCAGTGCCACGGCCAGGAGGACGGCGGCCA
GACCCCCGTGATGGCCAGCCCCTGCTGCGGCTGCTGCTGCGGCGACAGGAGCAGCC
CCCCCACCACCCCCCTGAGGGCCCCCGACCCCAGCCCCGGCGGCGTGCCCCTGGAG
GCCAGCCTGTGCCCCGCCAGCCTGGCCCCCAGCGGCATCAGCGAGAAGAGCAAGAG
CAGCAGCAGCTTCCACCCCGCCCCCGGCAACGCCCAGAGCAGCAGCCAGACCCCCA
AGATCGTGAACTTCGTGAGCGTGGGCCCCACCTACATGAGGGTGAGC
SEQ ID NO: 84 IL12R beta 1 extracellular (hinge)
WSQPQRFSIEVQVSD
SEQ ID NO: 85 IL12R beta 1 extracellular (hinge)
TGGAGCCAGCCCCAGAGGTTCAGCATCGAGGTGCAGGTGAGCGAC
SEQ ID NO: 86 IL12R beta 1 transmembrane
WLIFFASLGSFLSILLVGVLGYLGL
SEQ ID NO: 87 IL12R beta 1 transmembrane
TGGCTGATCTTCTTCGCCAGCCTGGGCAGCTTCCTGAGCATCCTGCTGGTGGGCGTG
CTGGGCTACCTGGGCCTG
SEQ ID NO: 88 IL12R beta 1 intracellular
NRAARHLCPPLPTPCASSAIEFPGGKETWQWINPVDFQEEASLQEALVVEMSWDKGERT
EPLEKTELPEGAPELALDTELSLEDGDRCKAKM
SEQ ID NO: 89 IL12R beta 1 intracellular
AACAGGGCCGCCAGGCACCTGTGCCCCCCCCTGCCCACCCCCTGCGCCAGCAGCGC
CATCGAGTTCCCCGGCGGCAAGGAGACCTGGCAGTGGATCAACCCCGTGGACTTCC
AGGAGGAGGCCAGCCTGCAGGAGGCCCTGGTGGTGGAGATGAGCTGGGACAAGGG
CGAGAGGACCGAGCCCCTGGAGAAGACCGAGCTGCCCGAGGGCGCCCCCGAGCTG
GCCCTGGACACCGAGCTGAGCCTGGAGGACGGCGACAGGTGCAAGGCCAAGATG
SEQ ID NO: 90 IL12R beta 1 extracellular, transmembrane, intracellula
WSQPQRFSIEVQVSDWLIFFASLGSFLSILLVGVLGYLGLNRAARHLCPPLPTPCASSAIEF
PGGKETWQWINPVDFQEEASLQEALVVEMSWDKGERTEPLEKTELPEGAPELALDTELS
LEDGDRCKAKM
SEQ ID NO: 91 IL12R beta 1 extracellular, transmembrane, intracellular
TGGAGCCAGCCCCAGAGGTTCAGCATCGAGGTGCAGGTGAGCGACTGGCTGATCTT
CTTCGCCAGCCTGGGCAGCTTCCTGAGCATCCTGCTGGTGGGCGTGCTGGGCTACCT
GGGCCTGAACAGGGCCGCCAGGCACCTGTGCCCCCCCCTGCCCACCCCCTGCGCCA
GCAGCGCCATCGAGTTCCCCGGCGGCAAGGAGACCTGGCAGTGGATCAACCCCGTG
GACTTCCAGGAGGAGGCCAGCCTGCAGGAGGCCCTGGTGGTGGAGATGAGCTGGGA
CAAGGGCGAGAGGACCGAGCCCCTGGAGAAGACCGAGCTGCCCGAGGGCGCCCCC
GAGCTGGCCCTGGACACCGAGCTGAGCCTGGAGGACGGCGACAGGTGCAAGGCCA
AGATG
SEQ ID NO: 92 ZipIL12R: EE2Xzip.IL12Rb1.2A.RR2Xzip.IL12Rb2.2A.mClover_IL12Rb1 chain
MDPDLEIEAAFLERENTALETRVAELRQRVQRLRNRVSQYRTRYGPLGGGKGGGGSGG
GGSGGGGSMDPDLEIEAAFLERENTALETRVAELRQRVQRLRNRVSQYRTRYGPLGGG
KWSQPQRFSIEVQVSDWLIFFASLGSFLSILLVGVLGYLGLNRAARHLCPPLPTPCASSAI
EFPGGKETWQWINPVDFQEEASLQEALVVEMSWDKGERTEPLEKTELPEGAPELALDTE
LSLEDGDRCKAKM
SEQ ID NO: 93 ZipIL 12R: EE2Xzip.IL12Rb1.2A.RR2Xzip.IL12Rb2.2A.mClover IL12Rb1 chain
ATGGACCCCGACCTGGAAATCGAGGCCGCCTTCCTGGAAAGAGAGAACACAGCCCT
GGAAACCAGAGTGGCCGAGCTGAGACAGAGAGTGCAGCGGCTGAGAAACCGGGTG
TCCCAGTACAGAACCAGATACGGACCTCTCGGCGGAGGAAAAGGTGGCGGAGGTTC
TGGCGGAGGCGGCTCTGGTGGTGGTGGATCTATGGATCCTGACCTCGAGATTGAAGC
CGCCTTTCTCGAGCGCGAGAATACCGCTCTCGAGACAAGAGTGGCTGAACTGCGGC
AGAGGGTCCAGAGACTGAGGAACAGAGTGTCTCAGTACCGGACACGCTACGGCCCT
CTTGGAGGTGGAAAATGGAGCCAGCCCCAGAGGTTCAGCATCGAGGTGCAGGTGAG
CGACTGGCTGATCTTCTTCGCCAGCCTGGGCAGCTTCCTGAGCATCCTGCTGGTGGG
CGTGCTGGGCTACCTGGGCCTGAACAGGGCCGCCAGGCACCTGTGCCCCCCCCTGCC
CACCCCCTGCGCCAGCAGCGCCATCGAGTTCCCCGGCGGCAAGGAGACCTGGCAGT
GGATCAACCCCGTGGACTTCCAGGAGGAGGCCAGCCTGCAGGAGGCCCTGGTGGTG
GAGATGAGCTGGGACAAGGGCGAGAGGACCGAGCCCCTGGAGAAGACCGAGCTGC
CCGAGGGCGCCCCCGAGCTGGCCCTGGACACCGAGCTGAGCCTGGAGGACGGCGAC
AGGTGCAAGGCCAAGATG
SEQ ID NO: 94 IL12R beta 2 extracellular (hinge)
SHGNEREFCLQGKAN
SEQ ID NO: 95 IL12R beta 2 extracellular (hinge)
AGCCACGGCAACGAGAGGGAGTTCTGCCTGCAGGGCAAGGCCAAC
SEQ ID NO: 96 IL12R beta 2 transmembrane
WMAFVAPSICIAIIMVGIFST
SEQ ID NO: 97 IL12R beta 2 transmembrane
TGGATGGCCTTCGTGGCCCCCAGCATCTGCATCGCCATCATCATGGTGGGCATCTTC
AGCACC
SEQ ID NO: 98 IL12R beta 2 intracellular
HYFQQKVFVLLAALRPQWCSREIPDPANSTCAKKYPIAEEKTQLPLDRLLIDWPTPEDPE
PLVISEVLHQVTPVFRHPPCSNWPQREKGIQGHQASEKDMMHSASSPPPPRALQAESRQ
LVDLYKVLESRGSDPKPENPACPWTVLPAGDLPTHDGYLPSNIDDLPSHEAPLADSLEEL
EPQHISLSVFPSSSLHPLTFSCGDKLTLDQLKMRCDSLML
SEQ ID NO: 99 IL12R beta 2 intracellular
CACTACTTCCAGCAGAAGGTGTTCGTGCTGCTGGCCGCCCTGAGGCCCCAGTGGTGC
AGCAGGGAGATCCCCGACCCCGCCAACAGCACCTGCGCCAAGAAGTACCCCATCGC
CGAGGAGAAGACCCAGCTGCCCCTGGACAGGCTGCTGATCGACTGGCCCACCCCCG
AGGACCCCGAGCCCCTGGTGATCAGCGAGGTGCTGCACCAGGTGACCCCCGTGTTC
AGGCACCCCCCCTGCAGCAACTGGCCCCAGAGGGAGAAGGGCATCCAGGGCCACCA
GGCCAGCGAGAAGGACATGATGCACAGCGCCAGCAGCCCCCCCCCCCCCAGGGCCC
TGCAGGCCGAGAGCAGGCAGCTGGTGGACCTGTACAAGGTGCTGGAGAGCAGGGGC
AGCGACCCCAAGCCCGAGAACCCCGCCTGCCCCTGGACCGTGCTGCCCGCCGGCGA
CCTGCCCACCCACGACGGCTACCTGCCCAGCAACATCGACGACCTGCCCAGCCACG
AGGCCCCCCTGGCCGACAGCCTGGAGGAGCTGGAGCCCCAGCACATCAGCCTGAGC
GTGTTCCCCAGCAGCAGCCTGCACCCCCTGACCTTCAGCTGCGGCGACAAGCTGACC
CTGGACCAGCTGAAGATGAGGTGCGACAGCCTGATGCTG
SEQ ID NO: 100 IL12R beta 2 extracellular, transmembrane, intracellular
SHGNEREFCLQGKANWMAFVAPSICIAIIMVGIFSTHYFQQKVFVLLAALRPQWCSREIP
DPANSTCAKKYPIAEEKTQLPLDRLLIDWPTPEDPEPLVISEVLHQVTPVFRHPPCSNWPQ
REKGIQGHQASEKDMMHSASSPPPPRALQAESRQLVDLYKVLESRGSDPKPENPACPWT
VLPAGDLPTHDGYLPSNIDDLPSHEAPLADSLEELEPQHISLSVFPSSSLHPLTFSCGDKLT
LDQLKMRCDSLML
SEQ ID NO: 101 IIL12R beta 2 extracellular, transmembrane, intracellular
AGCCACGGCAACGAGAGGGAGTTCTGCCTGCAGGGCAAGGCCAACTGGATGGCCTT
CGTGGCCCCCAGCATCTGCATCGCCATCATCATGGTGGGCATCTTCAGCACCCACTA
CTTCCAGCAGAAGGTGTTCGTGCTGCTGGCCGCCCTGAGGCCCCAGTGGTGCAGCAG
GGAGATCCCCGACCCCGCCAACAGCACCTGCGCCAAGAAGTACCCCATCGCCGAGG
AGAAGACCCAGCTGCCCCTGGACAGGCTGCTGATCGACTGGCCCACCCCCGAGGAC
CCCGAGCCCCTGGTGATCAGCGAGGTGCTGCACCAGGTGACCCCCGTGTTCAGGCA
CCCCCCCTGCAGCAACTGGCCCCAGAGGGAGAAGGGCATCCAGGGCCACCAGGCCA
GCGAGAAGGACATGATGCACAGCGCCAGCAGCCCCCCCCCCCCCAGGGCCCTGCAG
GCCGAGAGCAGGCAGCTGGTGGACCTGTACAAGGTGCTGGAGAGCAGGGGCAGCG
ACCCCAAGCCCGAGAACCCCGCCTGCCCCTGGACCGTGCTGCCCGCCGGCGACCTG
CCCACCCACGACGGCTACCTGCCCAGCAACATCGACGACCTGCCCAGCCACGAGGC
CCCCCTGGCCGACAGCCTGGAGGAGCTGGAGCCCCAGCACATCAGCCTGAGCGTGT
TCCCCAGCAGCAGCCTGCACCCCCTGACCTTCAGCTGCGGCGACAAGCTGACCCTGG
ACCAGCTGAAGATGAGGTGCGACAGCCTGATGCTG
SEQ ID NO: 102 ZipIL12R: EE2Xzip.IL12Rb1.2A.RR2Xzip.IL12Rb2.2A.mClover_IL12Rb2 chain
MDPDLEIRAAFLRQRNTALRTEVAELEQEVQRLENEVSQYETRYGPLGGGKGGGGSGG
GGSGGGGSMDPDLEIRAAFLRQRNTALRTEVAELEQEVQRLENEVSQYETRYGPLGGG
KSHGNEREFCLQGKANWMAFVAPSICIAIIMVGIFSTHYFQQKVFVLLAALRPQWCSREI
PDPANSTCAKKYPIAEEKTQLPLDRLLIDWPTPEDPEPLVISEVLHQVTPVFRHPPCSNWP
QREKGIQGHQASEKDMMHSASSPPPPRALQAESRQLVDLYKVLESRGSDPKPENPACP
WTVLPAGDLPTHDGYLPSNIDDLPSHEAPLADSLEELEPQHISLSVFPSSSLHPLTFSCGD
KLTLDQLKMRCDSLML
SEQ ID NO: 103 ZipIL12R: EE2Xzip.IL12Rb1.2A.RR2Xzip.IL12Rb2.2A.mClover_IL12Rb2 chain
ATGGACCCCGACCTGGAAATCAGAGCCGCCTTCCTGCGGCAGAGAAACACAGCCCT
GAGAACAGAGGTGGCCGAGCTGGAACAAGAGGTGCAGCGGCTGGAAAACGAGGTG
TCCCAGTACGAGACAAGATACGGCCCTCTCGGCGGAGGAAAAGGTGGCGGAGGTTC
TGGCGGAGGCGGAAGTGGTGGTGGCGGATCTATGGATCCTGACCTCGAGATTAGGG
CCGCCTTTCTGAGACAGCGGAACACCGCTCTGAGGACCGAAGTGGCCGAACTCGAG
CAAGAAGTCCAGAGACTGGAAAATGAAGTCTCTCAGTACGAAACCCGCTACGGACC
TCTTGGAGGCGGCAAAAGCCACGGCAACGAGAGGGAGTTCTGCCTGCAGGGCAAGG
CCAACTGGATGGCCTTCGTGGCCCCCAGCATCTGCATCGCCATCATCATGGTGGGCA
TCTTCAGCACCCACTACTTCCAGCAGAAGGTGTTCGTGCTGCTGGCCGCCCTGAGGC
CCCAGTGGTGCAGCAGGGAGATCCCCGACCCCGCCAACAGCACCTGCGCCAAGAAG
TACCCCATCGCCGAGGAGAAGACCCAGCTGCCCCTGGACAGGCTGCTGATCGACTG
GCCCACCCCCGAGGACCCCGAGCCCCTGGTGATCAGCGAGGTGCTGCACCAGGTGA
CCCCCGTGTTCAGGCACCCCCCCTGCAGCAACTGGCCCCAGAGGGAGAAGGGCATC
CAGGGCCACCAGGCCAGCGAGAAGGACATGATGCACAGCGCCAGCAGCCCCCCCCC
CCCCAGGGCCCTGCAGGCCGAGAGCAGGCAGCTGGTGGACCTGTACAAGGTGCTGG
AGAGCAGGGGCAGCGACCCCAAGCCCGAGAACCCCGCCTGCCCCTGGACCGTGCTG
CCCGCCGGCGACCTGCCCACCCACGACGGCTACCTGCCCAGCAACATCGACGACCT
GCCCAGCCACGAGGCCCCCCTGGCCGACAGCCTGGAGGAGCTGGAGCCCCAGCACA
TCAGCCTGAGCGTGTTCCCCAGCAGCAGCCTGCACCCCCTGACCTTCAGCTGCGGCG
ACAAGCTGACCCTGGACCAGCTGAAGATGAGGTGCGACAGCCTGATGCTG
SEQ ID NO: 104 IL23R extracellular (hinge)
ISTGHLTSDNRGDIG
SEQ ID NO: 105 IL23R extracellular (hinge)
ATCAGCACCGGCCACCTGACCAGCGACAACAGGGGCGACATCGGC
SEQ ID NO: 106 IL23R transmembrane
LLLGMIVFAVMLSILSLIGIF
SEQ ID NO: 107 IL23R transmembrane
CTGCTGCTGGGCATGATCGTGTTCGCCGTGATGCTGAGCATCCTGAGCCTGATCGGC
ATCTTC
SEQ ID NO: 108 IL23R intracellular
NRSFRTGIKRRILLLIPKWLYEDIPNMKNSNVVKMLQENSELMNNNS
SEQVLYVDPMITEIKEIFIPEHKPTDYKKENTGPLETRDYPQNSLFDNTTVVYIPDLNTGY
KPQISNFLPEGSHLSNNNEITSLTLKPPVDSLDSGNNPRLQKHPNFAFSVSSVNSLSNTIFL
GELSLILNQGECSSPDIQNSVEEETTMLLENDSPSETIPEQTLLPDEFVSCLGIVNEELPSIN
TYFPQNILESHENRISLLEK
SEQ ID NO: 109 IL23R intracellular
AACAGGAGCTTCAGGACCGGCATCAAGAGGAGGATCCTGCTGCTGATCCCCAAGTG
GCTGTACGAGGACATCCCCAACATGAAGAACAGCAACGTGGTGAAGATGCTGCAGG
AGAACAGCGAGCTGATGAACAACAACAGCAGCGAGCAGGTGCTGTACGTGGACCCC
ATGATCACCGAGATCAAGGAGATCTTCATCCCCGAGCACAAGCCCACCGACTACAA
GAAGGAGAACACCGGCCCCCTGGAGACCAGGGACTACCCCCAGAACAGCCTGTTCG
ACAACACCACCGTGGTGTACATCCCCGACCTGAACACCGGCTACAAGCCCCAGATC
AGCAACTTCCTGCCCGAGGGCAGCCACCTGAGCAACAACAACGAGATCACCAGCCT
GACCCTGAAGCCCCCCGTGGACAGCCTGGACAGCGGCAACAACCCCAGGCTGCAGA
AGCACCCCAACTTCGCCTTCAGCGTGAGCAGCGTGAACAGCCTGAGCAACACCATC
TTCCTGGGCGAGCTGAGCCTGATCCTGAACCAGGGCGAGTGCAGCAGCCCCGACAT
CCAGAACAGCGTGGAGGAGGAGACCACCATGCTGCTGGAGAACGACAGCCCCAGC
GAGACCATCCCCGAGCAGACCCTGCTGCCCGACGAGTTCGTGAGCTGCCTGGGCAT
CGTGAACGAGGAGCTGCCCAGCATCAACACCTACTTCCCCCAGAACATCCTGGAGA
GCCACTTCAACAGGATCAGCCTGCTGGAGAAG
SEQ ID NO: 110 IL23R extracellular, transmembrane, intracellular
ISTGHLTSDNRGDIGLLLGMIVFAVMLSILSLIGIFNRSFRTGIKRRILLLIPKWLYEDIPNM
KNSNVVKMLQENSELMNNNS
SEQVLYVDPMITEIKEIFIPEHKPTDYKKENTGPLETRDYPQNSLFDNTTVVYIPDLNTGY
KPQISNFLPEGSHLSNNNEITSLTLKPPVDSLDSGNNPRLQKHPNFAFSVSSVNSLSNTIFL
GELSLILNQGECSSPDIQNSVEEETTMLLENDSPSETIPEQTLLPDEFVSCLGIVNEELPSIN
TYFPQNILESHENRISLLEK
SEQ ID NO: 111 IL23R extracellular, transmembrane, intracellular
ATCAGCACCGGCCACCTGACCAGCGACAACAGGGGCGACATCGGCCTGCTGCTGGG
CATGATCGTGTTCGCCGTGATGCTGAGCATCCTGAGCCTGATCGGCATCTTCAACAG
GAGCTTCAGGACCGGCATCAAGAGGAGGATCCTGCTGCTGATCCCCAAGTGGCTGT
ACGAGGACATCCCCAACATGAAGAACAGCAACGTGGTGAAGATGCTGCAGGAGAA
CAGCGAGCTGATGAACAACAACAGCAGCGAGCAGGTGCTGTACGTGGACCCCATGA
TCACCGAGATCAAGGAGATCTTCATCCCCGAGCACAAGCCCACCGACTACAAGAAG
GAGAACACCGGCCCCCTGGAGACCAGGGACTACCCCCAGAACAGCCTGTTCGACAA
CACCACCGTGGTGTACATCCCCGACCTGAACACCGGCTACAAGCCCCAGATCAGCA
ACTTCCTGCCCGAGGGCAGCCACCTGAGCAACAACAACGAGATCACCAGCCTGACC
CTGAAGCCCCCCGTGGACAGCCTGGACAGCGGCAACAACCCCAGGCTGCAGAAGCA
CCCCAACTTCGCCTTCAGCGTGAGCAGCGTGAACAGCCTGAGCAACACCATCTTCCT
GGGCGAGCTGAGCCTGATCCTGAACCAGGGCGAGTGCAGCAGCCCCGACATCCAGA
ACAGCGTGGAGGAGGAGACCACCATGCTGCTGGAGAACGACAGCCCCAGCGAGAC
CATCCCCGAGCAGACCCTGCTGCCCGACGAGTTCGTGAGCTGCCTGGGCATCGTGAA
CGAGGAGCTGCCCAGCATCAACACCTACTTCCCCCAGAACATCCTGGAGAGCCACTT
CAACAGGATCAGCCTGCTGGAGAAG
SEQ ID NO: 112 ZipIL23R: EE2Xzip.IL12Rb1.2A.RR2Xzip.IL23R.2A.mClover_IL23R chain
MDPDLEIRAAFLRQRNTALRTEVAELEQEVQRLENEVSQYETRYGPLGGGKGGGGSGG
GGSGGGGSMDPDLEIRAAFLRQRNTALRTEVAELEQEVQRLENEVSQYETRYGPLGGG
KISTGHLTSDNRGDIGLLLGMIVFAVMLSILSLIGIFNRSFRTGIKRRILLLIPKWLYEDIPN
MKNSNVVKMLQENSELMNNNS
SEQVLYVDPMITEIKEIFIPEHKPTDYKKENTGPLETRDYPQNSLFDNTTVVYIPDLNTGY
KPQISNFLPEGSHLSNNNEITSLTLKPPVDSLDSGNNPRLQKHPNFAFSVSSVNSLSNTIFL
GELSLILNQGECSSPDIQNSVEEETTMLLENDSPSETIPEQTLLPDEFVSCLGIVNEELPSIN
TYFPQNILESHENRISLLEK
SEQ ID NO: 113 ZipIL23R: EE2Xzip.IL12Rb1.2A.RR2Xzip.IL23R.2A.mClover_IL23R chain
ATGGACCCCGACCTGGAAATCAGAGCCGCCTTCCTGCGGCAGAGAAACACAGCCCT
GAGAACAGAGGTGGCCGAGCTGGAACAAGAGGTGCAGCGGCTGGAAAACGAGGTG
TCCCAGTACGAGACAAGATACGGCCCTCTCGGCGGAGGAAAAGGTGGCGGAGGTTC
TGGCGGAGGCGGAAGTGGTGGTGGCGGATCTATGGATCCTGACCTCGAGATTAGGG
CCGCCTTTCTGAGACAGCGGAACACCGCTCTGAGGACCGAAGTGGCCGAACTCGAG
CAAGAAGTCCAGAGACTGGAAAATGAAGTCTCTCAGTACGAAACCCGCTACGGACC
TCTTGGAGGCGGCAAAATCAGCACCGGCCACCTGACCAGCGACAACAGGGGCGACA
TCGGCCTGCTGCTGGGCATGATCGTGTTCGCCGTGATGCTGAGCATCCTGAGCCTGA
TCGGCATCTTCAACAGGAGCTTCAGGACCGGCATCAAGAGGAGGATCCTGCTGCTG
ATCCCCAAGTGGCTGTACGAGGACATCCCCAACATGAAGAACAGCAACGTGGTGAA
GATGCTGCAGGAGAACAGCGAGCTGATGAACAACAACAGCAGCGAGCAGGTGCTGT
ACGTGGACCCCATGATCACCGAGATCAAGGAGATCTTCATCCCCGAGCACAAGCCC
ACCGACTACAAGAAGGAGAACACCGGCCCCCTGGAGACCAGGGACTACCCCCAGA
ACAGCCTGTTCGACAACACCACCGTGGTGTACATCCCCGACCTGAACACCGGCTACA
AGCCCCAGATCAGCAACTTCCTGCCCGAGGGCAGCCACCTGAGCAACAACAACGAG
ATCACCAGCCTGACCCTGAAGCCCCCCGTGGACAGCCTGGACAGCGGCAACAACCC
CAGGCTGCAGAAGCACCCCAACTTCGCCTTCAGCGTGAGCAGCGTGAACAGCCTGA
GCAACACCATCTTCCTGGGCGAGCTGAGCCTGATCCTGAACCAGGGCGAGTGCAGC
AGCCCCGACATCCAGAACAGCGTGGAGGAGGAGACCACCATGCTGCTGGAGAACG
ACAGCCCCAGCGAGACCATCCCCGAGCAGACCCTGCTGCCCGACGAGTTCGTGAGC
TGCCTGGGCATCGTGAACGAGGAGCTGCCCAGCATCAACACCTACTTCCCCCAGAA
CATCCTGGAGAGCCACTTCAACAGGATCAGCCTGCTGGAGAAG
SEQ ID NO: 114 IL10R alpha extracellular (hinge)
EECISLTRQYFTVTN
SEQ ID NO: 115 IL10R alpha extracellular (hinge)
GAGGAGTGCATCAGCCTGACCAGGCAGTACTTCACCGTGACCAAC
SEQ ID NO: 116 IL10R alpha transmembrane
VIIFFAFVLLLSGALAYCLAL
SEQ ID NO: 117 IL10R alpha transmembrane
GTGATCATCTTCTTCGCCTTCGTGCTGCTGCTGAGCGGCGCCCTGGCCTACTGCCTGG
CCCTG
SEQ ID NO: 118 IL10R alpha intracellular
QLYVRRRKKLPSVLLFKKPSPFIFISQRPSPETQDTIHPLDEEAFLKVSPELKNLDLHGSTD
SGFGSTKPSLQTEEPQFLLPDPHPQADRTLGNREPPVLGDSCSSGSSNSTDSGICLQEPSLS
PSTGPTWEQQVGSNSRGQDDSGIDLVQNSEGRAGDTQGGSALGHHSPPEPEVPGEEDPA
AVAFQGYLRQTRCAEEKATKTGCLEEESPLTDGLGPKFGRCLVDEAGLHPPALAKGYL
KQDPLEMTLASSGAPTGQWNQPTEEWSLLALSSCSDLGISDWSFAHDLAPLGCVAAPG
GLLGSFNSDLVTLPLISSLQSSE
SEQ ID NO: 119 IL10R alpha intracellular
CAGCTGTACGTGAGGAGGAGGAAGAAGCTGCCCAGCGTGCTGCTGTTCAAGAAGCC
CAGCCCCTTCATCTTCATCAGCCAGAGGCCCAGCCCCGAGACCCAGGACACCATCCA
CCCCCTGGACGAGGAGGCCTTCCTGAAGGTGAGCCCCGAGCTGAAGAACCTGGACC
TGCACGGCAGCACCGACAGCGGCTTCGGCAGCACCAAGCCCAGCCTGCAGACCGAG
GAGCCCCAGTTCCTGCTGCCCGACCCCCACCCCCAGGCCGACAGGACCCTGGGCAA
CAGGGAGCCCCCCGTGCTGGGCGACAGCTGCAGCAGCGGCAGCAGCAACAGCACCG
ACAGCGGCATCTGCCTGCAGGAGCCCAGCCTGAGCCCCAGCACCGGCCCCACCTGG
GAGCAGCAGGTGGGCAGCAACAGCAGGGGCCAGGACGACAGCGGCATCGACCTGG
TGCAGAACAGCGAGGGCAGGGCCGGCGACACCCAGGGCGGCAGCGCCCTGGGCCA
CCACAGCCCCCCCGAGCCCGAGGTGCCCGGCGAGGAGGACCCCGCCGCCGTGGCCT
TCCAGGGCTACCTGAGGCAGACCAGGTGCGCCGAGGAGAAGGCCACCAAGACCGG
CTGCCTGGAGGAGGAGAGCCCCCTGACCGACGGCCTGGGCCCCAAGTTCGGCAGGT
GCCTGGTGGACGAGGCCGGCCTGCACCCCCCCGCCCTGGCCAAGGGCTACCTGAAG
CAGGACCCCCTGGAGATGACCCTGGCCAGCAGCGGCGCCCCCACCGGCCAGTGGAA
CCAGCCCACCGAGGAGTGGAGCCTGCTGGCCCTGAGCAGCTGCAGCGACCTGGGCA
TCAGCGACTGGAGCTTCGCCCACGACCTGGCCCCCCTGGGCTGCGTGGCCGCCCCCG
GCGGCCTGCTGGGCAGCTTCAACAGCGACCTGGTGACCCTGCCCCTGATCAGCAGCC
TGCAGAGCAGCGAG
SEQ ID NO: 120 IL10R alpha extracellular, transmembrane, intracellular
EECISLTRQYFTVTNVIIFFAFVLLLSGALAYCLALQLYVRRRKKLPSVLLFKKPSPFIFISQ
RPSPETQDTIHPLDEEAFLKVSPELKNLDLHGSTDSGFGSTKPSLQTEEPQFLLPDPHPQA
DRTLGNREPPVLGDSCSSGSSNSTDSGICLQEPSLSPSTGPTWEQQVGSNSRGQDDSGIDL
VQNSEGRAGDTQGGSALGHHSPPEPEVPGEEDPAAVAFQGYLRQTRCAEEKATKTGCL
EEESPLTDGLGPKFGRCLVDEAGLHPPALAKGYLKQDPLEMTLASSGAPTGQWNQPTEE
WSLLALSSCSDLGISDWSFAHDLAPLGCVAAPGGLLGSFNSDLVTLPLISSLQSSE
SEQ ID NO: 121 IL10R alpha extracellular, transmembrane, intracellular
GAGGAGTGCATCAGCCTGACCAGGCAGTACTTCACCGTGACCAACGTGATCATCTTC
TTCGCCTTCGTGCTGCTGCTGAGCGGCGCCCTGGCCTACTGCCTGGCCCTGCAGCTG
TACGTGAGGAGGAGGAAGAAGCTGCCCAGCGTGCTGCTGTTCAAGAAGCCCAGCCC
CTTCATCTTCATCAGCCAGAGGCCCAGCCCCGAGACCCAGGACACCATCCACCCCCT
GGACGAGGAGGCCTTCCTGAAGGTGAGCCCCGAGCTGAAGAACCTGGACCTGCACG
GCAGCACCGACAGCGGCTTCGGCAGCACCAAGCCCAGCCTGCAGACCGAGGAGCCC
CAGTTCCTGCTGCCCGACCCCCACCCCCAGGCCGACAGGACCCTGGGCAACAGGGA
GCCCCCCGTGCTGGGCGACAGCTGCAGCAGCGGCAGCAGCAACAGCACCGACAGCG
GCATCTGCCTGCAGGAGCCCAGCCTGAGCCCCAGCACCGGCCCCACCTGGGAGCAG
CAGGTGGGCAGCAACAGCAGGGGCCAGGACGACAGCGGCATCGACCTGGTGCAGA
ACAGCGAGGGCAGGGCCGGCGACACCCAGGGCGGCAGCGCCCTGGGCCACCACAG
CCCCCCCGAGCCCGAGGTGCCCGGCGAGGAGGACCCCGCCGCCGTGGCCTTCCAGG
GCTACCTGAGGCAGACCAGGTGCGCCGAGGAGAAGGCCACCAAGACCGGCTGCCTG
GAGGAGGAGAGCCCCCTGACCGACGGCCTGGGCCCCAAGTTCGGCAGGTGCCTGGT
GGACGAGGCCGGCCTGCACCCCCCCGCCCTGGCCAAGGGCTACCTGAAGCAGGACC
CCCTGGAGATGACCCTGGCCAGCAGCGGCGCCCCCACCGGCCAGTGGAACCAGCCC
ACCGAGGAGTGGAGCCTGCTGGCCCTGAGCAGCTGCAGCGACCTGGGCATCAGCGA
CTGGAGCTTCGCCCACGACCTGGCCCCCCTGGGCTGCGTGGCCGCCCCCGGCGGCCT
GCTGGGCAGCTTCAACAGCGACCTGGTGACCCTGCCCCTGATCAGCAGCCTGCAGA
GCAGCGAG
SEQ ID NO: 122 ZipIL10R: EE2Xzip.IL10Ra.2A.RR2Xzip.IL10Rb.2A.mClover_IL10Ra chain
MDPDLEIEAAFLERENTALETRVAELRQRVQRLRNRVSQYRTRYGPLGGGKGGGGSGG
GGSGGGGSMDPDLEIEAAFLERENTALETRVAELRQRVQRLRNRVSQYRTRYGPLGGG
KEECISLTRQYFTVTNVIIFFAFVLLLSGALAYCLALQLYVRRRKKLPSVLLFKKPSPFIFIS
QRPSPETQDTIHPLDEEAFLKVSPELKNLDLHGSTDSGFGSTKPSLQTEEPQFLLPDPHPQ
ADRTLGNREPPVLGDSCSSGSSNSTDSGICLQEPSLSPSTGPTWEQQVGSNSRGQDDSGI
DLVQNSEGRAGDTQGGSALGHHSPPEPEVPGEEDPAAVAFQGYLRQTRCAEEKATKTG
CLEEESPLTDGLGPKFGRCLVDEAGLHPPALAKGYLKQDPLEMTLASSGAPTGQWNQP
TEEWSLLALSSCSDLGISDWSFAHDLAPLGCVAAPGGLLGSFNSDLVTLPLISSLQSSE
SEQ ID NO: 123 ZipIL10R: EE2Xzip.IL10Ra.2A.RR2Xzip.IL10Rb.2A.mClover IL10Ra chain
ATGGACCCCGACCTGGAAATCGAGGCCGCCTTCCTGGAAAGAGAGAACACAGCCCT
GGAAACCAGAGTGGCCGAGCTGAGACAGAGAGTGCAGCGGCTGAGAAACCGGGTG
TCCCAGTACAGAACCAGATACGGACCTCTCGGCGGAGGAAAAGGTGGCGGAGGTTC
TGGCGGAGGCGGCTCTGGTGGTGGTGGATCTATGGATCCTGACCTCGAGATTGAAGC
CGCCTTTCTCGAGCGCGAGAATACCGCTCTCGAGACAAGAGTGGCTGAACTGCGGC
AGAGGGTCCAGAGACTGAGGAACAGAGTGTCTCAGTACCGGACACGCTACGGCCCT
CTTGGAGGTGGAAAAGAGGAGTGCATCAGCCTGACCAGGCAGTACTTCACCGTGAC
CAACGTGATCATCTTCTTCGCCTTCGTGCTGCTGCTGAGCGGCGCCCTGGCCTACTGC
CTGGCCCTGCAGCTGTACGTGAGGAGGAGGAAGAAGCTGCCCAGCGTGCTGCTGTT
CAAGAAGCCCAGCCCCTTCATCTTCATCAGCCAGAGGCCCAGCCCCGAGACCCAGG
ACACCATCCACCCCCTGGACGAGGAGGCCTTCCTGAAGGTGAGCCCCGAGCTGAAG
AACCTGGACCTGCACGGCAGCACCGACAGCGGCTTCGGCAGCACCAAGCCCAGCCT
GCAGACCGAGGAGCCCCAGTTCCTGCTGCCCGACCCCCACCCCCAGGCCGACAGGA
CCCTGGGCAACAGGGAGCCCCCCGTGCTGGGCGACAGCTGCAGCAGCGGCAGCAGC
AACAGCACCGACAGCGGCATCTGCCTGCAGGAGCCCAGCCTGAGCCCCAGCACCGG
CCCCACCTGGGAGCAGCAGGTGGGCAGCAACAGCAGGGGCCAGGACGACAGCGGC
ATCGACCTGGTGCAGAACAGCGAGGGCAGGGCCGGCGACACCCAGGGCGGCAGCG
CCCTGGGCCACCACAGCCCCCCCGAGCCCGAGGTGCCCGGCGAGGAGGACCCCGCC
GCCGTGGCCTTCCAGGGCTACCTGAGGCAGACCAGGTGCGCCGAGGAGAAGGCCAC
CAAGACCGGCTGCCTGGAGGAGGAGAGCCCCCTGACCGACGGCCTGGGCCCCAAGT
TCGGCAGGTGCCTGGTGGACGAGGCCGGCCTGCACCCCCCCGCCCTGGCCAAGGGC
TACCTGAAGCAGGACCCCCTGGAGATGACCCTGGCCAGCAGCGGCGCCCCCACCGG
CCAGTGGAACCAGCCCACCGAGGAGTGGAGCCTGCTGGCCCTGAGCAGCTGCAGCG
ACCTGGGCATCAGCGACTGGAGCTTCGCCCACGACCTGGCCCCCCTGGGCTGCGTGG
CCGCCCCCGGCGGCCTGCTGGGCAGCTTCAACAGCGACCTGGTGACCCTGCCCCTGA
TCAGCAGCCTGCAGAGCAGCGAG
SEQ ID NO: 124 IL10R beta extracellular (hinge)
EPVCEQTTHDETVPS
SEQ ID NO: 125 IL10R beta extracellular (hinge)
GAGCCCGTGTGCGAGCAGACCACCCACGACGAGACCGTGCCCAGC
SEQ ID NO: 126 IL10R beta transmembrane
WMVAVILMASVFMVCLALLGCF
SEQ ID NO: 127 IL10R beta transmembrane
TGGATGGTGGCCGTGATCCTGATGGCCAGCGTGTTCATGGTGTGCCTGGCCCTGCTG
GGCTGCTTC
SEQ ID NO: 128 IL10R beta intracellular
ALLWCVYKKTKYAFSPRNSLPQHLKEFLGHPHHNTLLFFSFPLSDENDVFDKLSVIAEDS
ESGKQNPGDSCSLGTPPGQGPQS
SEQ ID NO: 129 IL10R beta intracellular
GCCCTGCTGTGGTGCGTGTACAAGAAGACCAAGTACGCCTTCAGCCCCAGGAACAG
CCTGCCCCAGCACCTGAAGGAGTTCCTGGGCCACCCCCACCACAACACCCTGCTGTT
CTTCAGCTTCCCCCTGAGCGACGAGAACGACGTGTTCGACAAGCTGAGCGTGATCGC
CGAGGACAGCGAGAGCGGCAAGCAGAACCCCGGCGACAGCTGCAGCCTGGGCACC
CCCCCCGGCCAGGGCCCCCAGAGC
SEQ ID NO: 130 IL10R beta extracellular, transmembrane, intracellular
EPVCEQTTHDETVPSWMVAVILMASVFMVCLALLGCFALLWCVYKKTKYAFSPRNSLP
QHLKEFLGHPHHNTLLFFSFPLSDENDVFDKLSVIAEDSESGKQNPGDSCSLGTPPGQGP
QS
SEQ ID NO: 131 IL10R beta extracellular, transmembrane, intracellular
GAGCCCGTGTGCGAGCAGACCACCCACGACGAGACCGTGCCCAGCTGGATGGTGGC
CGTGATCCTGATGGCCAGCGTGTTCATGGTGTGCCTGGCCCTGCTGGGCTGCTTCGC
CCTGCTGTGGTGCGTGTACAAGAAGACCAAGTACGCCTTCAGCCCCAGGAACAGCC
TGCCCCAGCACCTGAAGGAGTTCCTGGGCCACCCCCACCACAACACCCTGCTGTTCT
TCAGCTTCCCCCTGAGCGACGAGAACGACGTGTTCGACAAGCTGAGCGTGATCGCC
GAGGACAGCGAGAGCGGCAAGCAGAACCCCGGCGACAGCTGCAGCCTGGGCACCC
CCCCCGGCCAGGGCCCCCAGAGC
SEQ ID NO: 132 ZipIL10R: EE2Xzip.IL10Ra.2A.RR2Xzip.IL10Rb.2A.mClover_IL10Rb chain
MDPDLEIRAAFLRQRNTALRTEVAELEQEVQRLENEVSQYETRYGPLGGGKGGGGSGG
GGSGGGGSMDPDLEIRAAFLRQRNTALRTEVAELEQEVQRLENEVSQYETRYGPLGGG
KEPVCEQTTHDETVPSWMVAVILMASVFMVCLALLGCFALLWCVYKKTKYAFSPRNSL
PQHLKEFLGHPHHNTLLFFSFPLSDENDVFDKLSVIAEDSESGKQNPGDSCSLGTPPGQG
PQS
SEQ ID NO: 133 ZipIL10R: EE2Xzip.IL10Ra.2A.RR2Xzip.IL10Rb.2A.mClover_IL10Rb chain
ATGGACCCCGACCTGGAAATCAGAGCCGCCTTCCTGCGGCAGAGAAACACAGCCCT
GAGAACAGAGGTGGCCGAGCTGGAACAAGAGGTGCAGCGGCTGGAAAACGAGGTG
TCCCAGTACGAGACAAGATACGGCCCTCTCGGCGGAGGAAAAGGTGGCGGAGGTTC
TGGCGGAGGCGGAAGTGGTGGTGGCGGATCTATGGATCCTGACCTCGAGATTAGGG
CCGCCTTTCTGAGACAGCGGAACACCGCTCTGAGGACCGAAGTGGCCGAACTCGAG
CAAGAAGTCCAGAGACTGGAAAATGAAGTCTCTCAGTACGAAACCCGCTACGGACC
TCTTGGAGGCGGCAAAGAGCCCGTGTGCGAGCAGACCACCCACGACGAGACCGTGC
CCAGCTGGATGGTGGCCGTGATCCTGATGGCCAGCGTGTTCATGGTGTGCCTGGCCC
TGCTGGGCTGCTTCGCCCTGCTGTGGTGCGTGTACAAGAAGACCAAGTACGCCTTCA
GCCCCAGGAACAGCCTGCCCCAGCACCTGAAGGAGTTCCTGGGCCACCCCCACCAC
AACACCCTGCTGTTCTTCAGCTTCCCCCTGAGCGACGAGAACGACGTGTTCGACAAG
CTGAGCGTGATCGCCGAGGACAGCGAGAGCGGCAAGCAGAACCCCGGCGACAGCT
GCAGCCTGGGCACCCCCCCCGGCCAGGGCCCCCAGAGC
SEQ ID NO: 134 IL6R beta extracellular
EFTFTTPKFAQGEIE
SEQ ID NO: 135 IL6R beta extracellular
GAGTTCACCTTCACCACCCCCAAGTTCGCCCAGGGCGAGATCGAG
SEQ ID NO: 136 IL6R beta transmembrane
AIVVPVCLAFLLTTLLGVLFCF
SEQ ID NO: 137 IL6R beta transmembrane
GCCATCGTGGTGCCCGTGTGCCTGGCCTTCCTGCTGACCACCCTGCTGGGCGTGCTG
TTCTGCTTC
SEQ ID NO: 138 IL6R beta intracellular
NKRDLIKKHIWPNVPDPSKSHIAQWSPHTPPRHNFNSKDQMYSDGNFTDVSVVEIEAND
KKPFPEDLKSLDLFKKEKINTEGHSSGIGGSSCMSSSRPSISSSDENESSQNTSSTVQYSTV
VHSGYRHQVPSVQVFSRSESTQPLLDSEERPEDLQLVDHVDGGDGILPRQQYFKQNCSQ
HESSPDISHFERSKQVSSVNEEDFVRLKQQISDHISQSCGSGQMKMFQEVSAADAFGPGT
EGQVERFETVGMEAATDEGMPKSYLPQTVRQGGYMPQ
SEQ ID NO: 139 IL6R beta intracellular
AACAAGAGGGACCTGATCAAGAAGCACATCTGGCCCAACGTGCCCGACCCCAGCAA
GAGCCACATCGCCCAGTGGAGCCCCCACACCCCCCCCAGGCACAACTTCAACAGCA
AGGACCAGATGTACAGCGACGGCAACTTCACCGACGTGAGCGTGGTGGAGATCGAG
GCCAACGACAAGAAGCCCTTCCCCGAGGACCTGAAGAGCCTGGACCTGTTCAAGAA
GGAGAAGATCAACACCGAGGGCCACAGCAGCGGCATCGGCGGCAGCAGCTGCATG
AGCAGCAGCAGGCCCAGCATCAGCAGCAGCGACGAGAACGAGAGCAGCCAGAACA
CCAGCAGCACCGTGCAGTACAGCACCGTGGTGCACAGCGGCTACAGGCACCAGGTG
CCCAGCGTGCAGGTGTTCAGCAGGAGCGAGAGCACCCAGCCCCTGCTGGACAGCGA
GGAGAGGCCCGAGGACCTGCAGCTGGTGGACCACGTGGACGGCGGCGACGGCATCC
TGCCCAGGCAGCAGTACTTCAAGCAGAACTGCAGCCAGCACGAGAGCAGCCCCGAC
ATCAGCCACTTCGAGAGGAGCAAGCAGGTGAGCAGCGTGAACGAGGAGGACTTCGT
GAGGCTGAAGCAGCAGATCAGCGACCACATCAGCCAGAGCTGCGGCAGCGGCCAG
ATGAAGATGTTCCAGGAGGTGAGCGCCGCCGACGCCTTCGGCCCCGGCACCGAGGG
CCAGGTGGAGAGGTTCGAGACCGTGGGCATGGAGGCCGCCACCGACGAGGGCATGC
CCAAGAGCTACCTGCCCCAGACCGTGAGGCAGGGCGGCTACATGCCCCAG
SEQ ID NO: 140 IL6R beta extracellular, transmembrane, intracellular
EFTFTTPKFAQGEIEAIVVPVCLAFLLTTLLGVLFCFNKRDLIKKHIWPNVPDPSKSHIAQ
WSPHTPPRHNFNSKDQMYSDGNFTDVSVVEIEANDKKPFPEDLKSLDLFKKEKINTEGH
SSGIGGSSCMSSSRPSISSSDENESSQNTSSTVQYSTVVHSGYRHQVPSVQVFSRSESTQPL
LDSEERPEDLQLVDHVDGGDGILPRQQYFKQNCSQHESSPDISHFERSKQVSSVNEEDFV
RLKQQISDHISQSCGSGQMKMFQEVSAADAFGPGTEGQVERFETVGMEAATDEGMPKS
YLPQTVRQGGYMPQ
SEQ ID NO: 141 IL6R beta extracellular, transmembrane, intracellular
GAGTTCACCTTCACCACCCCCAAGTTCGCCCAGGGCGAGATCGAGGCCATCGTGGTG
CCCGTGTGCCTGGCCTTCCTGCTGACCACCCTGCTGGGCGTGCTGTTCTGCTTCAACA
AGAGGGACCTGATCAAGAAGCACATCTGGCCCAACGTGCCCGACCCCAGCAAGAGC
CACATCGCCCAGTGGAGCCCCCACACCCCCCCCAGGCACAACTTCAACAGCAAGGA
CCAGATGTACAGCGACGGCAACTTCACCGACGTGAGCGTGGTGGAGATCGAGGCCA
ACGACAAGAAGCCCTTCCCCGAGGACCTGAAGAGCCTGGACCTGTTCAAGAAGGAG
AAGATCAACACCGAGGGCCACAGCAGCGGCATCGGCGGCAGCAGCTGCATGAGCA
GCAGCAGGCCCAGCATCAGCAGCAGCGACGAGAACGAGAGCAGCCAGAACACCAG
CAGCACCGTGCAGTACAGCACCGTGGTGCACAGCGGCTACAGGCACCAGGTGCCCA
GCGTGCAGGTGTTCAGCAGGAGCGAGAGCACCCAGCCCCTGCTGGACAGCGAGGAG
AGGCCCGAGGACCTGCAGCTGGTGGACCACGTGGACGGCGGCGACGGCATCCTGCC
CAGGCAGCAGTACTTCAAGCAGAACTGCAGCCAGCACGAGAGCAGCCCCGACATCA
GCCACTTCGAGAGGAGCAAGCAGGTGAGCAGCGTGAACGAGGAGGACTTCGTGAG
GCTGAAGCAGCAGATCAGCGACCACATCAGCCAGAGCTGCGGCAGCGGCCAGATGA
AGATGTTCCAGGAGGTGAGCGCCGCCGACGCCTTCGGCCCCGGCACCGAGGGCCAG
GTGGAGAGGTTCGAGACCGTGGGCATGGAGGCCGCCACCGACGAGGGCATGCCCAA
GAGCTACCTGCCCCAGACCGTGAGGCAGGGCGGCTACATGCCCCAG
SEQ ID NO: 142 ZipIL27R: EE2Xzip.IL6Rb.2A.RR2Xzip.IL27Ra.2A.mClover_IL6Rb chain
MDPDLEIEAAFLERENTALETRVAELRQRVQRLRNRVSQYRTRYGPLGGGKGGGGSGG
GGSGGGGSMDPDLEIEAAFLERENTALETRVAELRQRVQRLRNRVSQYRTRYGPLGGG
KEFTFTTPKFAQGEIEAIVVPVCLAFLLTTLLGVLFCFNKRDLIKKHIWPNVPDPSKSHIA
QWSPHTPPRHNFNSKDQMYSDGNFTDVSVVEIEANDKKPFPEDLKSLDLFKKEKINTEG
HSSGIGGSSCMSSSRPSISSSDENESSQNTSSTVQYSTVVHSGYRHQVPSVQVFSRSESTQ
PLLDSEERPEDLQLVDHVDGGDGILPRQQYFKQNCSQHESSPDISHFERSKQVSSVNEED
FVRLKQQISDHISQSCGSGQMKMFQEVSAADAFGPGTEGQVERFETVGMEAATDEGMP
KSYLPQTVRQGGYMPQ
SEQ ID NO: 143 ZipIL27R: EE2Xzip.IL6Rb.2A.RR2Xzip.IL27Ra.2A.mClover IL6Rb chain
ATGGACCCCGACCTGGAAATCGAGGCCGCCTTCCTGGAAAGAGAGAACACAGCCCT
GGAAACCAGAGTGGCCGAGCTGAGACAGAGAGTGCAGCGGCTGAGAAACCGGGTG
TCCCAGTACAGAACCAGATACGGACCTCTCGGCGGAGGAAAAGGTGGCGGAGGTTC
TGGCGGAGGCGGCTCTGGTGGTGGTGGATCTATGGATCCTGACCTCGAGATTGAAGC
CGCCTTTCTCGAGCGCGAGAATACCGCTCTCGAGACAAGAGTGGCTGAACTGCGGC
AGAGGGTCCAGAGACTGAGGAACAGAGTGTCTCAGTACCGGACACGCTACGGCCCT
CTTGGAGGTGGAAAAGAGTTCACCTTCACCACCCCCAAGTTCGCCCAGGGCGAGAT
CGAGGCCATCGTGGTGCCCGTGTGCCTGGCCTTCCTGCTGACCACCCTGCTGGGCGT
GCTGTTCTGCTTCAACAAGAGGGACCTGATCAAGAAGCACATCTGGCCCAACGTGC
CCGACCCCAGCAAGAGCCACATCGCCCAGTGGAGCCCCCACACCCCCCCCAGGCAC
AACTTCAACAGCAAGGACCAGATGTACAGCGACGGCAACTTCACCGACGTGAGCGT
GGTGGAGATCGAGGCCAACGACAAGAAGCCCTTCCCCGAGGACCTGAAGAGCCTGG
ACCTGTTCAAGAAGGAGAAGATCAACACCGAGGGCCACAGCAGCGGCATCGGCGG
CAGCAGCTGCATGAGCAGCAGCAGGCCCAGCATCAGCAGCAGCGACGAGAACGAG
AGCAGCCAGAACACCAGCAGCACCGTGCAGTACAGCACCGTGGTGCACAGCGGCTA
CAGGCACCAGGTGCCCAGCGTGCAGGTGTTCAGCAGGAGCGAGAGCACCCAGCCCC
TGCTGGACAGCGAGGAGAGGCCCGAGGACCTGCAGCTGGTGGACCACGTGGACGGC
GGCGACGGCATCCTGCCCAGGCAGCAGTACTTCAAGCAGAACTGCAGCCAGCACGA
GAGCAGCCCCGACATCAGCCACTTCGAGAGGAGCAAGCAGGTGAGCAGCGTGAAC
GAGGAGGACTTCGTGAGGCTGAAGCAGCAGATCAGCGACCACATCAGCCAGAGCTG
CGGCAGCGGCCAGATGAAGATGTTCCAGGAGGTGAGCGCCGCCGACGCCTTCGGCC
CCGGCACCGAGGGCCAGGTGGAGAGGTTCGAGACCGTGGGCATGGAGGCCGCCACC
GACGAGGGCATGCCCAAGAGCTACCTGCCCCAGACCGTGAGGCAGGGCGGCTACAT
GCCCCAG
SEQ ID NO: 144 IL27R alpha extracellular
PILRLHLPDNTLRWK
SEQ ID NO: 145 IL27R alpha extracellular
CCCATCCTGAGGCTGCACCTGCCCGACAACACCCTGAGGTGGAAG
SEQ ID NO: 146 IL27R alpha transmembrane
VLPGILFLWGLFLLGCGLSLA
SEQ ID NO: 147 IL27R alpha transmembrane
GTGCTGCCCGGCATCCTGTTCCTGTGGGGCCTGTTCCTGCTGGGCTGCGGCCTGAGC
CTGGCC
SEQ ID NO: 148 IL27R alpha intracellular
TSGRCYHLRHKVLPRWVWEKVPDPANSSSGQPHMEQVPEAQPLGDLPILEVEEMEPPP
VMESSQPAQATAPLDSGYEKHFLPTPEELGLLGPPRPQVLAESGKQNPGDSCSLGTPPGQ
GPQS
SEQ ID NO: 149 IL27R alpha intracellular
ACCAGCGGCAGGTGCTACCACCTGAGGCACAAGGTGCTGCCCAGGTGGGTGTGGGA
GAAGGTGCCCGACCCCGCCAACAGCAGCAGCGGCCAGCCCCACATGGAGCAGGTGC
CCGAGGCCCAGCCCCTGGGCGACCTGCCCATCCTGGAGGTGGAGGAGATGGAGCCC
CCCCCCGTGATGGAGAGCAGCCAGCCCGCCCAGGCCACCGCCCCCCTGGACAGCGG
CTACGAGAAGCACTTCCTGCCCACCCCCGAGGAGCTGGGCCTGCTGGGCCCCCCCA
GGCCCCAGGTGCTGGCC
SEQ ID NO: 150 IL27R alpha extracellular, transmembrane, intracellular
PILRLHLPDNTLRWKVLPGILFLWGLFLLGCGLSLATSGRCYHLRHKVLPRWVWEKVPD
PANSSSGQPHMEQVPEAQPLGDLPILEVEEMEPPPVMESSQPAQATAPLDSGYEKHFLPT
PEELGLLGPPRPQVLAESGKQNPGDSCSLGTPPGQGPQS
SEQ ID NO: 151 IL27R alpha extracellular, transmembrane, intracellular
CCCATCCTGAGGCTGCACCTGCCCGACAACACCCTGAGGTGGAAGGTGCTGCCCGG
CATCCTGTTCCTGTGGGGCCTGTTCCTGCTGGGCTGCGGCCTGAGCCTGGCCACCAG
CGGCAGGTGCTACCACCTGAGGCACAAGGTGCTGCCCAGGTGGGTGTGGGAGAAGG
TGCCCGACCCCGCCAACAGCAGCAGCGGCCAGCCCCACATGGAGCAGGTGCCCGAG
GCCCAGCCCCTGGGCGACCTGCCCATCCTGGAGGTGGAGGAGATGGAGCCCCCCCC
CGTGATGGAGAGCAGCCAGCCCGCCCAGGCCACCGCCCCCCTGGACAGCGGCTACG
AGAAGCACTTCCTGCCCACCCCCGAGGAGCTGGGCCTGCTGGGCCCCCCCAGGCCC
CAGGTGCTGGCC
SEQ ID NO: 152 ZipIL27R: EE2Xzip.IL6Rb.2A.RR2Xzip.IL27Ra.2A.mClover_IL27Ra chain
MDPDLEIRAAFLRQRNTALRTEVAELEQEVQRLENEVSQYETRYGPLGGGKGGGGSGG
GGSGGGGSMDPDLEIRAAFLRQRNTALRTEVAELEQEVQRLENEVSQYETRYGPLGGG
KPILRLHLPDNTLRWKVLPGILFLWGLFLLGCGLSLATSGRCYHLRHKVLPRWVWEKVP
DPANSSSGQPHMEQVPEAQPLGDLPILEVEEMEPPPVMESSQPAQATAPLDSGYEKHFLP
TPEELGLLGPPRPQVLAESGKQNPGDSCSLGTPPGQGPQS
SEQ ID NO: 153 ZipIL27R: EE2Xzip.IL6Rb.2A.RR2Xzip.IL27Ra.2A.mClover_IL27Ra chain
ATGGACCCCGACCTGGAAATCAGAGCCGCCTTCCTGCGGCAGAGAAACACAGCCCT
GAGAACAGAGGTGGCCGAGCTGGAACAAGAGGTGCAGCGGCTGGAAAACGAGGTG
TCCCAGTACGAGACAAGATACGGCCCTCTCGGCGGAGGAAAAGGTGGCGGAGGTTC
TGGCGGAGGCGGAAGTGGTGGTGGCGGATCTATGGATCCTGACCTCGAGATTAGGG
CCGCCTTTCTGAGACAGCGGAACACCGCTCTGAGGACCGAAGTGGCCGAACTCGAG
CAAGAAGTCCAGAGACTGGAAAATGAAGTCTCTCAGTACGAAACCCGCTACGGACC
TCTTGGAGGCGGCAAACCCATCCTGAGGCTGCACCTGCCCGACAACACCCTGAGGT
GGAAGGTGCTGCCCGGCATCCTGTTCCTGTGGGGCCTGTTCCTGCTGGGCTGCGGCC
TGAGCCTGGCCACCAGCGGCAGGTGCTACCACCTGAGGCACAAGGTGCTGCCCAGG
TGGGTGTGGGAGAAGGTGCCCGACCCCGCCAACAGCAGCAGCGGCCAGCCCCACAT
GGAGCAGGTGCCCGAGGCCCAGCCCCTGGGCGACCTGCCCATCCTGGAGGTGGAGG
AGATGGAGCCCCCCCCCGTGATGGAGAGCAGCCAGCCCGCCCAGGCCACCGCCCCC
CTGGACAGCGGCTACGAGAAGCACTTCCTGCCCACCCCCGAGGAGCTGGGCCTGCT
GGGCCCCCCCAGGCCCCAGGTGCTGGCC
SEQ ID NO: 154 ZipIL2/21R: EE2Xzip.IL2Rb.IL21R.2A.RR2Xzip.IL2Rg.2A.mClover_IL2Rb.
IL21R chain
MDPDLEIEAAFLERENTALETRVAELRQRVQRLRNRVSQYRTRYGPLGGGKGGGGSGG
GGSGGGGSMDPDLEIEAAFLERENTALETRVAELRQRVQRLRNRVSQYRTRYGPLGGG
KPLAFRTKPAALGKDTIPWLGHLLVGLSGAFGFIILVYLLINCRNTGPWLKKVLKCNTPD
PSKFFSQLSSEHGGDVQKWLSSPFPSSSFSPGGLAPEISPLEVLERDKVTQLLLQQDKVPE
PASLSSNHSLTSCFTNQGYFFFHLPDALEIEACQVYFTYDPYSEEDPDEGVAGAPTGSSPQ
PLQPLSGEDDAYCTFPSRDDLLLFSPSLLGGPSPPSTAPGGSGAGEERMPPSLQERVPRD
WDPQPLGPPTPGVPDLVDFQPPPELVLREAGEEVPDAGPREGVSFPWSRPPGQGEFRAL
NARLPLNTDAYLSLQELQGQDPTHLVSDPVIFQTQSEELKEGWNPHLLLLLLLVIVFIPAF
WSLKTHPLWRLWKKIWAVPSPERFFMPLYKGCSGDFKKWVGAPFTGSSLELGPWSPEV
PSTLEVYSCHPPRSPAKRLQLTELQEPAELVESDGVPKPSFWPTAQNSGGSAYSEERDRP
YGLVSIDTVTVLDAEGPCTWPCSCEDDGYPALDLDAGLEPSPGLEDPLLDAGTTVLSCG
CVSAGSPGLGGPLGSLLDRLKPPLADGEDWAGGLPWGGRSPGGVSESEAGSPLAGLDM
DTFDSGFVGSDCSSPVECDFTSPGDEGPPRSYLRQWVVIPPPLSSPGPQAS
SEQ ID NO: 155 ZipIL2/21R: EE2Xzip.IL2Rb.IL21R.2A.RR2Xzip.IL2Rg.2A.mClover_IL2Rb.
IL21R chain
ATGGACCCCGACCTGGAAATCGAGGCCGCCTTCCTGGAAAGAGAGAACACAGCCCT
GGAAACCAGAGTGGCCGAGCTGAGACAGAGAGTGCAGCGGCTGAGAAACCGGGTG
TCCCAGTACAGAACCAGATACGGACCTCTCGGCGGAGGAAAAGGTGGCGGAGGTTC
TGGCGGAGGCGGCTCTGGTGGTGGTGGATCTATGGATCCTGACCTCGAGATTGAAGC
CGCCTTTCTCGAGCGCGAGAATACCGCTCTCGAGACAAGAGTGGCTGAACTGCGGC
AGAGGGTCCAGAGACTGAGGAACAGAGTGTCTCAGTACCGGACACGCTACGGCCCT
CTTGGAGGTGGAAAACCTCTGGCCTTCAGAACAAAACCTGCCGCTCTGGGCAAAGA
CACAATCCCCTGGCTGGGACATCTGCTCGTTGGACTGTCTGGCGCCTTCGGCTTCAT
CATCCTGGTGTACCTGCTGATCAACTGCCGGAACACAGGCCCTTGGCTGAAGAAAGT
GCTGAAGTGCAACACCCCTGATCCGAGCAAGTTCTTTAGCCAGCTGAGCAGCGAGC
ATGGCGGCGACGTTCAGAAATGGCTGTCTAGCCCATTTCCTAGCAGCAGCTTCAGCC
CTGGTGGACTGGCCCCTGAGATTAGCCCTCTGGAAGTGCTGGAACGGGACAAAGTG
ACCCAGCTGCTCCTGCAGCAGGATAAGGTGCCAGAACCTGCCAGCCTGTCCAGCAA
TCACAGCCTGACCAGCTGCTTTACCAACCAGGGCTACTTCTTCTTCCATCTGCCTGAC
GCTCTCGAGATCGAGGCCTGCCAGGTGTACTTCACCTACGATCCCTACAGCGAGGAA
GATCCCGACGAAGGCGTTGCAGGCGCTCCTACAGGATCTTCTCCACAGCCTCTGCAG
CCACTGTCCGGCGAGGATGATGCCTACTGCACCTTTCCAAGCCGGGACGACCTGCTG
CTGTTTAGCCCTAGTCTGCTCGGCGGACCTTCTCCACCATCTACAGCTCCAGGTGGA
AGCGGAGCCGGCGAAGAAAGAATGCCTCCTAGCCTGCAAGAGCGGGTGCCCAGAG
ATTGGGATCCTCAACCTCTCGGACCTCCTACACCAGGCGTGCCAGACCTGGTGGATT
TTCAGCCTCCTCCAGAGCTGGTGCTGAGAGAAGCTGGCGAAGAAGTGCCAGACGCC
GGACCTAGAGAGGGCGTTAGCTTTCCTTGGAGCAGACCTCCTGGACAGGGCGAGTT
CAGAGCCCTGAATGCTAGACTGCCCCTGAACACCGATGCCTACCTGTCTCTGCAAGA
ACTGCAGGGACAAGACCCCACACACCTCGTTGAGCGACCCCGTGATCTTTCAGACCC
AGAGCGAGGAACTGAAAGAAGGCTGGAACCCTCACCTCCTGCTGTTGCTGCTGCTG
GTCATCGTGTTCATCCCCGCCTTTTGGAGCCTGAAAACACACCCTCTGTGGCGGCTG
TGGAAGAAAATCTGGGCCGTGCCATCTCCTGAGCGGTTCTTCATGCCTCTGTACAAG
GGCTGCAGCGGCGACTTCAAGAAATGGGTCGGAGCCCCTTTTACCGGCAGCTCTCTG
GAACTTGGACCTTGGAGCCCTGAGGTGCCCAGCACACTGGAAGTGTACAGCTGTCA
CCCTCCTAGAAGCCCCGCCAAGAGACTGCAGCTGACAGAGCTGCAAGAGCCTGCCG
AGCTGGTGGAATCTGATGGCGTGCCCAAGCCTAGCTTCTGGCCCACAGCTCAGAATA
GCGGCGGCTCTGCCTACAGCGAGGAAAGAGATAGACCCTACGGCCTGGTGTCCATC
GACACCGTGACAGTGCTGGATGCCGAGGGACCTTGTACCTGGCCTTGTAGCTGCGAG
GACGATGGCTACCCTGCTCTGGATCTGGACGCTGGCCTTGAGCCTTCTCCAGGACTG
GAAGATCCTCTGCTGGACGCCGGAACAACCGTGCTGTCTTGTGGCTGTGTGTCTGCC
GGATCTCCTGGACTTGGAGGCCCTCTGGGAAGCCTGCTGGATAGACTGAAACCTCCT
CTGGCCGACGGCGAAGATTGGGCTGGTGGACTTCCTTGGGGCGGAAGATCTCCAGG
CGGAGTGTCTGAGTCTGAAGCCGGTTCTCCACTGGCCGGCCTGGACATGGATACCTT
CGATTCTGGCTTCGTGGGCAGCGACTGTAGCAGCCCTGTGGAATGCGACTTCACAAG
CCCTGGCGACGAGGGCCCACCTAGAAGCTATCTGAGACAGTGGGTCGTGATCCCTC
CACCTCTGTCTAGTCCTGGACCTCAGGCTTCT
SEQ ID NO: 156 ZipIL2/21R: EE2Xzip.IL2Rb.IL21R.2A.RR2Xzip.IL2Rg.2A.mClover_IL2Rg
chain
MDPDLEIRAAFLRQRNTALRTEVAELEQEVQRLENEVSQYETRYGPLGGGKGGGGSGG
GGSGGGGSMDPDLEIRAAFLRQRNTALRTEVAELEQEVQRLENEVSQYETRYGPLGGG
KSNTSKENPFLFALEAVVISVGSMGLIISLLCVYFWLERTMPRIPTLKNLEDLVTEYHGNF
SAWSGVSKGLAESLQPDYSERLCLVSEIPPKGGALGEGPGASPCNQHSPYWAPPCYTLK
PET
SEQ ID NO: 157 ZipIL2/21R: EE2Xzip.IL2Rb.IL21R.2A.RR2Xzip.IL2Rg.2A.mClover_IL2Rg
chain
ATGGACCCCGACCTGGAAATCAGAGCCGCCTTCCTGCGGCAGAGAAACACAGCCCT
GAGAACAGAGGTGGCCGAGCTGGAACAAGAGGTGCAGCGGCTGGAAAACGAGGTG
TCCCAGTACGAGACAAGATACGGCCCTCTCGGCGGAGGAAAAGGTGGCGGAGGTTC
TGGCGGAGGCGGAAGTGGTGGTGGCGGATCTATGGATCCTGACCTCGAGATTAGGG
CCGCCTTTCTGAGACAGCGGAACACCGCTCTGAGGACCGAAGTGGCCGAACTCGAG
CAAGAAGTCCAGAGACTGGAAAATGAAGTCTCTCAGTACGAAACCCGCTACGGACC
TCTTGGAGGCGGCAAAAGCAACACCAGCAAAGAGAACCCCTTCCTGTTCGCCCTGG
AAGCCGTGGTTATCAGCGTGGGCTCTATGGGCCTGATCATCAGCCTGCTGTGCGTGT
ACTTCTGGCTGGAACGGACCATGCCTCGGATCCCCACACTGAAGAACCTGGAAGAT
CTGGTCACCGAGTACCACGGCAACTTCTCTGCTTGGAGCGGCGTGTCAAAAGGCCTG
GCCGAATCTCTGCAGCCCGACTACTCTGAGAGACTGTGCCTGGTGTCTGAGATCCCT
CCTAAAGGCGGAGCCCTCGGAGAAGGACCTGGTGCCTCTCCATGTAACCAGCACAG
CCCTTATTGGGCCCCTCCTTGCTACACCCTGAAGCCTGAAACA
SEQ ID NO: 158 RR leucine zipper x2 with linker
MDPDLEIRAAFLRQRNTALRTEVAELEQEVQRLENEVSQYETRYGPLGGGKGGGGSGG
GGSGGGGSMDPDLEIRAAFLRQRNTALRTEVAELEQEVQRLENEVSQYETRYGPLGGG
K
SEQ ID NO: 159 RR leucine zipper x2 with linker
ATGGACCCCGACCTGGAAATCAGAGCCGCCTTCCTGCGGCAGAGAAACACAGCCCT
GAGAACAGAGGTGGCCGAGCTGGAACAAGAGGTGCAGCGGCTGGAAAACGAGGTG
TCCCAGTACGAGACAAGATACGGCCCTCTCGGCGGAGGAAAAGGTGGCGGAGGTTC
TGGCGGAGGCGGAAGTGGTGGTGGCGGATCTATGGATCCTGACCTCGAGATTAGGG
CCGCCTTTCTGAGACAGCGGAACACCGCTCTGAGGACCGAAGTGGCCGAACTCGAG
CAAGAAGTCCAGAGACTGGAAAATGAAGTCTCTCAGTACGAAACCCGCTACGGACC
TCTTGGAGGCGGCAAA
SEQ ID NO: 160 EE leucine zipper x2 with linker
MDPDLEIEAAFLERENTALETRVAELRQRVQRLRNRVSQYRTRYGPLGGGKGGGGSGG
GGSGGGGSMDPDLEIEAAFLERENTALETRVAELRQRVQRLRNRVSQYRTRYGPLGGG
K
SEQ ID NO: 161 EE leucine zipper x2 with linker
ATGGACCCCGACCTGGAAATCGAGGCCGCCTTCCTGGAAAGAGAGAACACAGCCCT
GGAAACCAGAGTGGCCGAGCTGAGACAGAGAGTGCAGCGGCTGAGAAACCGGGTG
TCCCAGTACAGAACCAGATACGGACCTCTCGGCGGAGGAAAAGGTGGCGGAGGTTC
TGGCGGAGGCGGCTCTGGTGGTGGTGGATCTATGGATCCTGACCTCGAGATTGAAGC
CGCCTTTCTCGAGCGCGAGAATACCGCTCTCGAGACAAGAGTGGCTGAACTGCGGC
AGAGGGTCCAGAGACTGAGGAACAGAGTGTCTCAGTACCGGACACGCTACGGCCCT
CTTGGAGGTGGAAAA
SEQ ID NO: 162 Leucine zipper amino acid sequence
DLERNRAAASRCRQKRKVWVQSLEKKAEDLSSLNGQLQSEVTLLRNEVAQLKQLLLAH
KDCPVTAMQKKSG
SEQ ID NO: 163 Leucine zipper amino acid sequence
KRKLWVSSLEKKAEELTSQNIQLSNEVTLLRNEVAQLKQLLLAHKDCPVTALQK
SEQ ID NO: 164 Leucine zipper amino acid sequence
QRKVWVMSLEKKAEELTQTNMQLQNEVSMLKNEVAQLKQLLLTHKDCPITAMQKES
SEQ ID NO: 165 Leucine zipper amino acid sequence
CPEEDERKKRRRERNKIAAAKCRNKKKEKTECLQKESEKLESVNAELKAQIEELKNEKQ
HLIYMLNLHRPTCIVRAQNGRTPED
SEQ ID NO: 166 Leucine zipper amino acid sequence
ERTEFLQRESERLELMNAELKTQIEELKQERQQLILMLNRHR
SEQ ID NO: 167 Leucine zipper amino acid sequence
NKTAATRYRQKKRAEQEALTGECKELEKKNEALKERADSLAKEIQYLKDLIEEVRKAR
GKKRVP
SEQ ID NO: 168 Leucine zipper amino acid sequence
KKGTRYRQKKRVEQEVLTGECKAVEKKNEALQERADSLAEEIQYMKDSIEEVCKARGK
KRVL
SEQ ID NO: 169 Leucine zipper amino acid sequence
NRKQKKRDQNKSAALRYRQRKRAEGEALEGECQGLEARNRELKERAESVEREIQYVKD
LLIEVYKARSQRTRSC
SEQ ID NO: 170 Leucine zipper amino acid sequence
ACQSRKKKKEYMLGLEARLKAALSENEQLKKENGTLKRQLDEVVSENQRLKVPSPKRR
V
SEQ ID NO: 171 Leucine zipper amino acid sequence
EYLQGLEARLQAVLADNQQLRRENAALRRRLEALLAENSELKLGSGNRK
SEQ ID NO: 172 Leucine zipper amino acid sequence
CRLNRLKKKEYVMGLESRVRGLAAENQELRAENRELGKRVQALQEESRYLRAVLANE
TGL
SEQ ID NO: 173 Leucine zipper amino acid sequence
QKADTLHLESEDLEKQNAALRKEIKQLTEELKYFTSVLNSHE
SEQ ID NO: 174 Leucine zipper amino acid sequence
RSRKKQTQKADKLHEEYESLEQENTMLRREIGKLTEELKHLTEALKEHEKMCPLLLCPM
NFVH
SEQ ID NO: 175 Leucine zipper amino acid sequence
DKADALHQQHESLEKDNLALRKEIQSLQAELAWWSRTLHVHE
SEQ ID NO: 176 Leucine zipper amino acid sequence
QRNVETQQKVLELTSDNDRLRKRVEQLSRELDTLRGIFRQL
SEQ ID NO: 177 Leucine zipper amino acid sequence
SDEYKIRRERNNIAVRKSRDKAKMRNLETQHKVLELTAENERLQKKVEQLSRELSTLRN
LFKQLPEPLLASSGHC
SEQ ID NO: 178 Leucine zipper amino acid sequence
GERNNMAVKKSRLKSKQKAQDTLQRVNQLKEENERLEAKIKLLTKELSVLKDLFLEHA
HNLADNVQSISTENTTADG
SEQ ID NO: 179 Leucine zipper amino acid sequence
RRNQEMQQKLVELSAENEKLHQRVEQLTRDLAGLRQFFKQL
SEQ ID NO: 180 Leucine zipper amino acid sequence
HLEYRLRRERNNIAVRKSRDKAKRRILETQQKVLEYMAENERLRSRVEQLTQELDTLRN
LFRQIPEAANLIKGV
SEQ ID NO: 181 Leucine zipper amino acid sequence
RMKEKEQENERKVAQLAEENERLKQEIERLTREVEATRRALIDRMVNLHQA
SEQ ID NO: 182 Leucine zipper amino acid sequence
QRNVETQQKVLELTSDNDRLRNGVEQLSRELDTLRGIFRQL
SEQ ID NO: 183 Leucine zipper amino acid sequence
QRKLETIVQLERELERLTNERERLLRARGEADRTLEVMRQQLTELYRDIFQHLRDESGNS
SEQ ID NO: 184 Leucine zipper amino acid sequence
KLDTILNLERDVEDLQRDKARLLREKVEFLRSLRQMKQKVQSLYQEVFGRLRDENGRP
Y
SEQ ID NO: 185 Leucine zipper amino acid sequence
FRDIRRRGKNKVAAQNCRKRKLENIVELEQDLDHLKDEKEKLLKEKGENDKSLHLLKK
QLSTLYLEVFSMLRD
SEQ ID NO: 186 Leucine zipper amino acid sequence
GDIRRRGKNKVAAQNCRKRKLDIILNLEDDVCNLQAKKETLKREQAQCNKAINIMKQK
LHDLYHDIFSRLRDDQGRPV
SEQ ID NO: 187 Leucine zipper amino acid sequence
GCRKRKLDCIQNLESEIEKLQSEKESLLKERDHILSTLGETKQNLTGLCQKVCKEAALSQ
EQ
SEQ ID NO: 188 Leucine zipper amino acid sequence
RKRKLDCIQNLECEIRKLVCEKEKLLSERNQLKACMGELLDNFSCLSQEVCRDIQ
SEQ ID NO: 189 Leucine zipper amino acid sequence
DPQLKREIRLMKNREAARECRRKKKEYVKCLENRVAVLENQNKTLIEELKTLKDLYSN
KSV
SEQ ID NO: 190 Leucine zipper amino acid sequence
KEEATRKRELRLMKNREAARECRRKKKEYVKCLENRVAVLENQNKTLIEELKALKDLY
CHKVE
SEQ ID NO: 191 Leucine zipper amino acid sequence
KEEATRKRELRLMKNREAARECRRKKKEYVKCLENRVAVLENQNKTLIEELKALRDLY
C
SEQ ID NO: 192 Leucine zipper amino acid sequence
EYVKCLESRVAVLEVQNKKLIEELETLKDICS
SEQ ID NO: 193 Leucine zipper amino acid sequence
KEAARKREVRLMKNREAARECRRKKKEYVKCLENRVAVLENQNKTLIEELKALKDLY
CHKSD
SEQ ID NO: 194 Leucine zipper amino acid sequence
EKRRLNDLVLENKLIALGEENATLKAELLSLKLKFGLISSTAYAQEIQKLSNSTAVYFQD
YQTSKSNV
SEQ ID NO: 195 Leucine zipper amino acid sequence
LNDAAIAGRLAALMEENALLKGELKALKLRFGLLPLT
SEQ ID NO: 196 Leucine zipper amino acid sequence
FRRERNKMAAAKCRNRRRELTDTLQAETDQLEDEKSALQTEIANLLKEKEKLEFILAAH
RPACKIPDDLGFPEEMS
SEQ ID NO: 197 Leucine zipper amino acid sequence
NRERNKLAAAKCRNRRRELTDRLQAETDQLEEEKAELESEIAELQKEKERLEFVLVAHK
PGCKIPYEA
SEQ ID NO: 198 Leucine zipper amino acid sequence
NRRKELTDFLQAETDKLEDEKSGLQREIEELQKQKERLELVLEAHRPICKIPEGAKEGDT
GSTS
SEQ ID NO: 199 Leucine zipper amino acid sequence
ERNKLAAAKCRNRRRELTEKLQAETEELEEEKSGLQKEIAELQKEKEKLEFMLVAHGPV
CKISP
SEQ ID NO: 200 Leucine zipper amino acid sequence
QRKLERIARLEEKVKTLKAQNSELASTANMLREQVAQLKQKVMNHVNSGCQLMLTQQ
Y
SEQ ID NO: 201 Leucine zipper amino acid sequence
NIKVERKRLRNRLAATKCRKRKLERIARLEDKVKTLKAENAGLSSTAGLLREQVAQLK
QKVMTHVSNGCQLLLGVKGHAFY
SEQ ID NO: 202 Leucine zipper amino acid sequence
DMDTQERIKAERKRLRNRIAASKCRKRKLERISRLEEKVKTLKSQNTELASTASLLREQV
AQLKQKVLSHVN
SEQ ID NO: 203 Leucine zipper amino acid sequence
QRVQQRHVLESEKNQLLQQVDHLKQEISRLVRERDAYKEKYEKLVSSGFRENGSSSDNP
SSPEFFM
SEQ ID NO: 204 Leucine zipper amino acid sequence
QYKRVQQKHHLENEKTQLIQQVEQLKQEVSRLARERDAYKVKCEKLANSGFREAGSTS
DSPSSPEFFL
SEQ ID NO: 205 Leucine zipper amino acid sequence
QQRHILESEKCQLQSQVEQLKLEVGRLAKERDLYKEKYEKLAGRGG
SEQ ID NO: 206 Leucine zipper amino acid sequence
QQRRGLEAERARLAAQLDALRAEVARLARERDLYKARCDRLTSSG
SEQ ID NO: 207 Leucine zipper amino acid sequence
EYVECLEKKVETFTSENNELWKKVETLENANRTLLQQLQKLQTLVTNKISR
SEQ ID NO: 208 Leucine zipper amino acid sequence
EYIDGLETRMSACTAQNQELQRKVLHLEKQNLSLLEQLKKLQAIVVQSTS
SEQ ID NO: 209 Leucine zipper amino acid sequence
WRRKIRNKRSAQESRRKKKVYVGGLESRVLKYTAQNMELQNKVQLLEEQNLSLLDQL
RKLQAMVIEISNKTSS
SEQ ID NO: 210 Leucine zipper amino acid sequence
WRKIRNKQSAQDSRRRKKEYIDGLESRVAACSAQNQELQKKVQELERHNISLVAQLRQ
LQTLIAQTSNKAAQTSTCVL
SEQ ID NO: 211 Leucine zipper amino acid sequence
EYMDSLEDDVESCSTENLELRKKVEVLENTNRASHGELV
SEQ ID NO: 212 Leucine zipper amino acid sequence
EEQKDEKYWSRRYKNNEAAKRSRDARRLKENQISVRAAFLEKENALLRQEVVAVRQEL
SHYRAVLSRYQAQHGAL
SEQ ID NO: 213 Leucine zipper amino acid sequence
LKENQIAIRASFLEKENSALRQEVADLRKELGKCKNILAKYEARH
SEQ ID NO: 214 Leucine zipper amino acid sequence
NWTRRKKNNVAAKRSRDARRLKENQITIRAAFLEKENTALRTEVAELRKEVGKCKTIVS
KYETKYGPL
SEQ ID NO: 215 Leucine zipper amino acid sequence
GVTQKEELEKQKAELQQEVEKLASENASMKLELDALRSKYEALQTFARTVARSPVAPA
RGPLAAGLGPLVPGKVAATSVITIVKSKTDA
SEQ ID NO: 216 Leucine zipper amino acid sequence
TQKEELERQRVELQQEVEKLARENSSMRLELDALRSKYEALQTFARTVAR
SEQ ID NO: 217 Leucine zipper amino acid sequence
CQKEELQKQKSELEREVDKLARENAAMRLELDALRGKCEALQGFARSVAAARG
SEQ ID NO: 218 Leucine zipper amino acid sequence
QTARDRKKARMSELEQQVVDLEEENQKLLLENQLLREKTHGLVVENQELRQRLGMDA
LVAEEEAEAKGNEV
SEQ ID NO: 219 Leucine zipper amino acid sequence
PSQNAFRKRKLERLEELEKKEAQLTVTNDQIHILKKENELLHFMLRSLLTERNMP
SEQ ID NO: 220 Leucine zipper amino acid sequence
KRAAQNRSAQKAFRQRREKYIKNLEEKSKLFDGLMKENSELKKMIESLKSKLKE
SEQ ID NO: 221 Leucine zipper amino acid sequence
EKRTAQNRAAQRAFRERKERKMKELEKKVQSLESIQQQNEVEATFLRDQLITLD
SEQ ID NO: 222 Leucine zipper amino acid sequence
EDTPPNPKKEIEFPGGSPGRKRIDSEAKSRRTAQNRAAQRAFRDRKEAKMKSLQERVEL
LEQKDAQNKTTTDFLLCSLKSLLSEITKYRAKNSDDERILAFLDDL
SEQ ID NO: 223 Leucine zipper amino acid sequence
EDTPPNPKKEIEFQAKKKAQNRAAQKAFRERKEARMKELQDKLLESERNRQSLLKEIEE
LRKANTEINAENRLLLRSGN
SEQ ID NO: 224 Leucine zipper amino acid sequence
EDTPPNPKKEIEFPKRQNRDAQRAYRERKNNKLQVLEETIESLSKVVKNYETKLNRLQN
ELQAKESENHALKQKLETLTLKQASVPAQDPILQN
SEQ ID NO: 225 Leucine zipper amino acid sequence
QQNRDAQRAYRERRTTRIQVLEEKVEMLHNLVDDWQRKYKLLESEFSDTKENLQKSIA
LNNELQKALPLIVNTPFQQQPEN
SEQ ID NO: 226 Leucine zipper amino acid sequence
EDTPPNPKKEIEFGIRKKQKNFENMNKLQNLNTQINKLRDRIEQLNKENEFWKAKLNDI
NEIKSLKLLNDIKRRNMG
SEQ ID NO: 227 Leucine zipper amino acid sequence
EDTPPNPKKEIEFHPAALKRARNTEAARRSRARKLQRMKQLEDKVEELLSKNYHLENEV
ARLKKLVGDAAR
SEQ ID NO: 228 Leucine zipper amino acid sequence
EDTPPNPKKEIEFDDDEDLLIQSKKSHQKKKLKEKELESSIHELTEIAASLQKRIHTLETEN
KLLKNLVLSSGET
SEQ ID NO: 229 T2A sequence
AEGRGSLLTCGDVEENPGP
T2A sequence
SEQ ID NO: 230
EGRGSLLTCGDVEENPGP
SEQ ID NO: 231 F2A sequence
NPGPGSGSRVTELLYRMKRAETYCPRPLLAIHPTEARHKQKIVAPVKQLLNFDLLKLAGDVES
SEQ ID NO: 232 Sponge (Amphimedon queenslandica) 2A sequence
LLCFLLLLLSGDVELNPGP
SEQ ID NO: 233 Sponge (Amphimedon queenslandica) 2A sequence
HHFMFLLLLLAGDIELNPGP
SEQ ID NO: 234 acorn worm 2A sequence
WFLVLLSFILSGDIEVNPGP
SEQ ID NO: 235 amphioxus (Branchiostoma floridae) 2A sequence
KNCAMYMLLLSGDVETNPGP
SEQ ID NO: 236 amphioxus (Branchiostoma floridae) 2A sequence
MVISQLMLKLAGDVEENPGP
SEQ ID NO: 237 equine rhinitis A virus 2A sequence
GSGQCTNYALLKLAGDVESNPGP
SEQ ID NO: 238 2A consensus sequence
D-X-E-X-NPGP (X is any amino acid residue)

1. A chimeric cytokine receptor comprising
a) a first polypeptide comprising:
i) a first extracellular region comprising one or more leucine zipper motifs,
ii) optionally, a first hinge region,
iii) a first transmembrane region, and
iv) at least a first intracellular signaling region derived from a first cytokine receptor chain; and
b) a second polypeptide comprising:
i) a second extracellular region comprising one or more leucine zipper motifs,
ii) optionally, a second hinge region,
iii) a second transmembrane region, and
iv) at least a second intracellular signaling region derived from a second cytokine receptor chain,
wherein the one or more leucine zipper motifs from the first extracellular region heterodimerize with the one or more leucine zipper motifs from the second extracellular region.

2. The chimeric cytokine receptor of claim 1, wherein each extracellular region comprises two leucine zipper motifs.

3. The chimeric cytokine receptor of claim 1 or 2, wherein each leucine zipper motif of the first or second extracellular region comprises at least five heptad repeats of amino acids with a leucine at every seventh position.

4. The chimeric cytokine receptor of any one of claims 1-3, wherein a) each leucine zipper motif of the first extracellular region comprises the amino acid sequence of LEIEAAFLERENTALETRVAELRQRVQRLRNRVSQYRTRYGPL (SEQ ID NO: 1), and each leucine zipper motif of the second extracellular region comprises the amino acid sequence of LEIRAAFLRQRNTALRTEVAELEQEVQRLENEVSQYETRYGPL (SEQ ID NO: 2); or b) each leucine zipper motif of the first extracellular region comprises the amino acid sequence of LEIRAAFLRQRNTALRTEVAELEQEVQRLENEVSQYETRYGPL (SEQ ID NO: 2), and each leucine zipper motif of the second extracellular region comprises the amino acid sequence of
(SEQ ID NO: 1)
LEIEAAFLERENTALETRVAELRQRVQRLRNRVSQYRTRYGPL.

5. The chimeric cytokine receptor of any one of claims 1-4, wherein the at least two leucine zipper motifs are operatively linked to each other via a linker.

6. The chimeric cytokine receptor of claim 5, wherein the linker comprises the amino acid sequence of GGGGSGGGGSGGGGS (SEQ ID NO: 11).

7. The chimeric cytokine receptor of any one of claims 1-6, wherein the at least one first and/or second intracellular signaling regions are derived from cytokine receptor chain(s) of a type I cytokine receptor.

8. The chimeric cytokine receptor of any one of claims 1-6, wherein the at least one first and/or second intracellular signaling regions are derived from cytokine receptor chain(s) of a type II cytokine receptor.

9. The chimeric cytokine receptor of any one of claims 1-6, wherein the at least one first and/or second intracellular signaling regions are derived from cytokine receptor chain(s) of the IL-2 receptor, IL-7 receptor, IL-15 receptor, IL-21 receptor, IL-12 receptor, IL-9 receptor, IL-4 receptor, IL-23 receptor, IL-10 receptor, or IL-27 receptor, or a combination thereof.

10. The chimeric cytokine receptor of any one of claims 1-7, and 9, wherein the at least one first and/or second intracellular signaling regions are derived from cytokine receptor chain(s) of a gamma cytokine receptor.

11. The chimeric cytokine receptor of claim 10, wherein the first or the second intracellular signaling region is derived from the common gamma chain (γc) (also known as IL-2 receptor gamma chain or IL-2RG, or CD132).

12. The chimeric cytokine receptor of any one of claims 1-11, wherein the first intracellular signaling region is derived from the common gamma chain (γc), and the second intracellular signaling region is derived from the IL-2 receptor beta chain.

13. The chimeric cytokine receptor of any one of claims 1-11, wherein the first intracellular signaling region is derived from the common gamma chain (γc), and the second intracellular signaling region is derived from the IL-7 receptor alpha chain.

14. The chimeric cytokine receptor of claim any one of claims 1-11, wherein the first intracellular signaling region is derived from the common gamma chain (γc), and the second intracellular signaling region is derived from the IL-21 receptor chain.

15. The chimeric cytokine receptor of any one of claims 1-11, wherein the first intracellular signaling region is derived from the common gamma chain (γc), and the second intracellular signaling region is derived from the IL-9 receptor chain.

16. The chimeric cytokine receptor of any one of claims 1-11, wherein the first intracellular signaling region is derived from the common gamma chain (γc), and the second intracellular signaling region is derived from the IL-4 receptor alpha chain.

17. The chimeric cytokine receptor of any one of claims 1-9, wherein the first intracellular signaling region is derived from the IL-12 receptor beta 1 chain, and the second intracellular signaling region is derived from the IL-12 receptor beta 2 chain.

18. The chimeric cytokine receptor of any one of claims 1-9, wherein the first intracellular signaling region is derived from the IL-23 receptor chain, and the second intracellular signaling region is derived from the IL-12 receptor beta 2 chain.

19. The chimeric cytokine receptor of any one of claims 1-9, wherein the first intracellular signaling region is derived from the IL-10 receptor alpha chain, and the second intracellular signaling region is derived from the IL-10 receptor beta chain.

20. The chimeric cytokine receptor of any one of claims 1-9, wherein the first intracellular signaling region is derived from the IL-27 receptor alpha chain (or WSX-1), and the second intracellular signaling region is derived from glycoprotein 130 (gp130 or IL-6 beta chain).

21. The chimeric cytokine receptor of any one of claims 1-11, wherein the first and/or the second polypeptides comprise two intracellular signaling regions derived from two cytokine receptor chains.

22. The chimeric cytokine receptor of claim 21, wherein the first polypeptide comprises an intracellular signaling region derived from the IL-2 receptor beta chain and an intracellular signaling region derived from the IL-21 receptor chain, and the second polypeptide comprises an intracellular signaling region derived from the common gamma chain (γc).

23. The chimeric cytokine receptor of claim 21, wherein the first polypeptide comprises an intracellular signaling region derived from the IL-7 receptor alpha chain and an intracellular signaling region derived from the IL-21 receptor chain, and the second polypeptide comprises an intracellular signaling region derived from the common gamma chain (γc).

24. The chimeric cytokine receptor of any one of claims 1-23, wherein the first hinge region is derived from the same first cytokine receptor chain as the first intracellular signaling region.

25. The chimeric cytokine receptor of any one of claims 1-24, wherein the second hinge region is derived from the same second cytokine receptor chain as the second intracellular signaling region.

26. The chimeric cytokine receptor of claim 24 or 25, wherein the first and/or second hinge regions are derived from cytokine receptor chain(s) of the IL-2 receptor, IL-7 receptor, IL-15 receptor, IL-21 receptor, IL-12 receptor, IL-9 receptor, IL-4 receptor, IL-23 receptor, IL-10 receptor, or IL-27 receptor.

27. The chimeric cytokine receptor of claim 26, wherein the first and/or second hinge regions are derived from cytokine receptor chain(s) of a gamma cytokine receptor.

28. The chimeric cytokine receptor of claim 27, wherein the first or the second hinge region is derived from the common gamma chain (γc).

29. The chimeric cytokine receptor of any one of claims 1-23, wherein the first and/or second hinge regions are derived from a molecule different from the first and/or second cytokine receptor chain(s) from which the first and/or second intracellular signaling region(s) is derived.

30. The chimeric cytokine receptor of claim 29, wherein the first and/or second hinge regions are derived from IgG1, IgG2, IgG3, IgG4, CD28, or CD8a.

31. The chimeric cytokine receptor of any one of claims 1-30, wherein the first and second hinge regions are the same.

32. The chimeric cytokine receptor of any one of claims 1-30, wherein the first and second hinge regions are different.

33. The chimeric cytokine receptor of any one of claims 1-32, wherein the first transmembrane region is derived from the same cytokine receptor chain as the first intracellular signaling region.

34. The chimeric cytokine receptor of any one of claims 1-32, wherein the second transmembrane region is derived from the same cytokine receptor chain as the second intracellular signaling region.

35. The chimeric cytokine receptor of claim 33 or 34, wherein the first and/or second transmembrane regions are derived from cytokine receptor chain(s) of the IL-2 receptor, IL-7 receptor, IL-15 receptor, IL-21 receptor, IL-12 receptor, IL-9 receptor, IL-4 receptor, IL-23 receptor, IL-10 receptor, or IL-27 receptor.

36. The chimeric cytokine receptor of claim 35, wherein the first and/or second transmembrane regions are derived from a gamma cytokine receptor.

37. The chimeric cytokine receptor of claim 36, wherein the first or the second transmembrane region is derived from the common gamma chain (γc).

38. The chimeric cytokine receptor of any one of claims 1-32, wherein the first and/or second transmembrane regions are derived from a molecule different from the first and/or second cytokine receptor chain(s) from which the first and/or second intracellular signaling region(s) is derived.

39. The chimeric cytokine receptor of claim 38, wherein the first and/or second transmembrane regions are derived from CD28, CD8, CD4, CD3ζ, CD40, CD134 (OX-40), CD19, or CD7.

40. The chimeric cytokine receptor of any one of claims 1-39, wherein the first and second transmembrane regions are the same.

41. The chimeric cytokine receptor of any one of claims 1-39, wherein the first and second transmembrane regions are different.

42. The chimeric cytokine receptor of claim 1, comprising
a) a first polypeptide comprising:
i) a first extracellular region comprising one or more leucine zipper motifs,
ii) optionally, a first hinge region derived from the common gamma chain (γc),
iii) a first transmembrane region derived from the common gamma chain (γc), and
iv) a first intracellular signaling region derived from the common gamma chain (γc); and
b) a second polypeptide comprising:
i) a second extracellular region comprising one or more leucine zipper motifs,
ii) optionally, a second hinge region derived from the IL-2 receptor beta chain,
iii) a second transmembrane region derived from the IL-2 receptor beta chain, and
iv) a second intracellular signaling region derived from the IL-2 receptor beta chain.

43. The chimeric cytokine receptor of claim 1, comprising
a) a first polypeptide comprising:
i) a first extracellular region comprising at least one or more leucine zipper motifs,
ii) optionally, a first hinge region derived from the common gamma chain (γc),
iii) a first transmembrane region derived from the common gamma chain (γc), and
iv) a first intracellular signaling region derived from the common gamma chain (γc); and
b) a second polypeptide comprising:
i) a second extracellular region comprising one or more leucine zipper motifs,
ii) optionally, a second hinge region derived from the IL-7 receptor alpha chain,
iii) a second transmembrane region derived from the IL-7 receptor alpha chain, and
iv) a second intracellular signaling region derived from the IL-7 receptor alpha chain.

44. The chimeric cytokine receptor of claim 1, comprising
a) a first polypeptide comprising:
i) a first extracellular region comprising one or more leucine zipper motifs,
ii) optionally, a first hinge region derived from the common gamma chain (γc),
iii) a first transmembrane region derived from the common gamma chain (γc), and
iv) a first intracellular signaling region derived from the common gamma chain (γc); and
b) a second polypeptide comprising:
i) a second extracellular region comprising one or more leucine zipper motifs,
ii) optionally, a second hinge region derived from the IL-21 receptor chain,
iii) a second transmembrane region derived from the IL-21 receptor chain, and
iv) a second intracellular signaling region derived from the IL-21 receptor chain.

45. The chimeric cytokine receptor of claim 1, comprising
a) a first polypeptide comprising:
i) a first extracellular region comprising one or more leucine zipper motifs,
ii) optionally, a first hinge region derived from the common gamma chain (γc),
iii) a first transmembrane region derived from the common gamma chain (γc), and
iv) a first intracellular signaling region derived from the common gamma chain (γc); and
b) a second polypeptide comprising:
i) a second extracellular region comprising one or more leucine zipper motifs,
ii) optionally, a second hinge region derived from the IL-9 receptor chain,
iii) a second transmembrane region derived from the IL-9 receptor chain, and
iv) a second intracellular signaling region derived from the IL-9 receptor chain.

46. The chimeric cytokine receptor of claim 1, comprising
a) a first polypeptide comprising:
i) a first extracellular region comprising one or more leucine zipper motifs,
ii) optionally, a first hinge region derived from the common gamma chain (γc),
iii) a first transmembrane region derived from the common gamma chain (γc), and
iv) a first intracellular signaling region derived from the common gamma chain (γc); and
b) a second polypeptide comprising:
i) a second extracellular region comprising one or more leucine zipper motifs,
ii) optionally, a second hinge region derived from the IL-4 receptor alpha chain,
iii) a second transmembrane region derived from the IL-4 receptor alpha chain, and
iv) a second intracellular signaling region derived from the IL-4 receptor alpha chain.

47. The chimeric cytokine receptor of claim 1, comprising
a) a first polypeptide comprising:
i) a first extracellular region comprising one or more leucine zipper motifs,
ii) optionally, a first hinge region derived from the IL-12 receptor beta 1 chain,
iii) a first transmembrane region derived from the IL-12 receptor beta 1 chain, and
iv) a first intracellular signaling region derived from the IL-12 receptor beta 1 chain; and
b) a second polypeptide comprising:
i) a second extracellular region comprising one or more leucine zipper motifs,
ii) optionally, a second hinge region derived from the IL-12 receptor beta 2 chain,
iii) a second transmembrane region derived from the IL-12 receptor beta 2 chain, and
iv) a second intracellular signaling region derived from the IL-12 receptor beta 2 chain.

48. The chimeric cytokine receptor of claim 1, comprising
a) a first polypeptide comprising:
i) a first extracellular region comprising one or more leucine zipper motifs,
ii) optionally, a first hinge region derived from the IL-23 receptor chain,
iii) a first transmembrane region derived from the IL-23 receptor chain, and
iv) a first intracellular signaling region derived from the IL-23 receptor chain; and
b) a second polypeptide comprising:
i) a second extracellular region comprising one or more leucine zipper motifs,
ii) optionally, a second hinge region derived from the IL-12 receptor beta 2 chain,
iii) a second transmembrane region derived from the IL-12 receptor beta 2 chain, and
iv) a second intracellular signaling region derived from the IL-12 receptor beta 2 chain.

49. The chimeric cytokine receptor of claim 1, comprising
a) a first polypeptide comprising:
i) a first extracellular region comprising one or more leucine zipper motifs,
ii) optionally, a first hinge region derived from the IL-10 receptor alpha chain,
iii) a first transmembrane region derived from the IL-10 receptor alpha chain, and
iv) a first intracellular signaling region derived from the IL-10 receptor alpha chain; and
b) a second polypeptide comprising:
i) a second extracellular region comprising one or more leucine zipper motifs,
ii) optionally, a second hinge region derived from the IL-10 receptor beta chain,
iii) a second transmembrane region derived from the IL-10 receptor beta chain, and
iv) a second intracellular signaling region derived from the IL-10 receptor beta chain.

50. The chimeric cytokine receptor of claim 1, comprising
a) a first polypeptide comprising:
i) a first extracellular region comprising one or more leucine zipper motifs,
ii) optionally, a first hinge region derived from the IL-27 receptor alpha chain (or WSX-1),
iii) a first transmembrane region derived from the IL-27 receptor alpha chain (or WSX-1), and
iv) a first intracellular signaling region derived from the IL-27 receptor alpha chain (or WSX-1); and
b) a second polypeptide comprising:
i) a second extracellular region comprising one or more leucine zipper motifs,
ii) optionally, a second hinge region derived from glycoprotein 130 (gp130),
iii) a second transmembrane region derived from glycoprotein 130 (gp130), and
iv) a second intracellular signaling region derived from glycoprotein 130 (gp130).

51. The chimeric cytokine receptor of claim 1, comprising
a) a first polypeptide comprising:
i) a first extracellular region comprising one or more leucine zipper motifs,
ii) optionally, a first hinge region derived from the common gamma chain (γc),
iii) a first transmembrane region derived from the common gamma chain (γc), and
iv) a first intracellular signaling region derived from the common gamma chain (γc); and
b) a second polypeptide comprising:
i) a second extracellular region comprising one or more leucine zipper motifs,
ii) optionally, a second hinge region derived from IL-2 receptor beta chain,
iii) a second transmembrane region derived from IL-2 receptor beta chain, and
iv) a second intracellular signaling region derived from IL-2 receptor beta chain; and
v) a third intracellular signaling region derived from the IL-21 receptor chain.

52. The chimeric cytokine receptor of any one of claims 11-16, 22, 23, 42-46, and 51, wherein the intracellular signaling region derived from the common gamma chain (γc) comprises the amino acid sequence of SEQ ID NO: 34, or an amino acid sequence having at least 80% identity thereof.

53. The chimeric cytokine receptor of any one of claims 28, 42-46, and 51, wherein the hinge region derived from the common gamma chain (γc) comprises the amino acid sequence of SEQ ID NO: 30, or a sequence having at least 80% identity thereof.

54. The chimeric cytokine receptor of any one of claims 37, 42-46, and 51, wherein the transmembrane region derived from the common gamma chain (γc) comprises the amino acid sequence of SEQ ID NO: 32, or a sequence having at least 80% identity thereof.

55. The chimeric cytokine receptor of any one of claims 12, 22, 42, and 51, wherein the intracellular signaling region derived from the IL-2 receptor beta chain comprises the amino acid sequence of SEQ ID NO: 18, or an amino acid sequence having at least 80% identity thereof.

56. The chimeric cytokine receptor of claim 42 or 51, wherein the hinge region derived from the IL-2 receptor beta chain comprises the amino acid sequence of SEQ ID NO: 14, or a sequence having at least 80% identity thereof.

57. The chimeric cytokine receptor of claim 42 or 51, wherein the transmembrane region derived from the IL-2 receptor beta chain comprises the amino acid sequence of SEQ ID NO: 16, or a sequence having at least 80% identity thereof.

58. The chimeric cytokine receptor of any one of claims 13, 23 or 43, wherein the intracellular signaling region derived from the IL-7 receptor alpha chain comprises the amino acid sequence of SEQ ID NO: 48, or an amino acid sequence having at least 80% identity thereof.

59. The chimeric cytokine receptor of claim 43, wherein the hinge region derived from the IL-7 receptor alpha chain comprises the amino acid sequence of SEQ ID NO: 44, or a sequence having at least 80% identity thereof.

60. The chimeric cytokine receptor of claim 43, wherein the transmembrane region derived from the IL-7 receptor alpha chain comprises the amino acid sequence of SEQ ID NO: 46, or a sequence having at least 80% identity thereof.

61. The chimeric cytokine receptor of any one of claims 14, 22, 23, 44, and 51, wherein the intracellular signaling region derived from the IL-21 receptor chain comprises the amino acid sequence of SEQ ID NO: 58, or an amino acid sequence having at least 80% identity thereof.

62. The chimeric cytokine receptor of claim 44, wherein the hinge region derived from the IL-21 receptor chain comprises the amino acid sequence of SEQ ID NO: 54, or a sequence having at least 80% identity thereof.

63. The chimeric cytokine receptor of claim 44, wherein the transmembrane region derived from the IL-21 receptor chain comprises the amino acid sequence of SEQ ID NO: 56, or a sequence having at least 80% identity thereof.

64. The chimeric cytokine receptor of any one of claims 15 and 45, wherein the intracellular signaling region derived from the TL-9 receptor chain comprises the amino acid sequence of SEQ ID NO: 68, or an amino acid sequence having at least 80% identity thereof.

65. The chimeric cytokine receptor of claim 45, wherein the hinge region derived from the IL-9 receptor chain comprises the amino acid sequence of SEQ ID NO: 64, or a sequence having at least 80% identity thereof.

66. The chimeric cytokine receptor of claim 45, wherein the transmembrane region derived from the TL-9 receptor chain comprises the amino acid sequence of SEQ ID NO: 66, or a sequence having at least 80% identity thereof.

67. The chimeric cytokine receptor of any one of claims 16 and 46, wherein the intracellular signaling region derived from the TL-4 receptor alpha chain comprises the amino acid sequence of SEQ ID NO: 78, or an amino acid sequence having at least 80% identity thereof.

68. The chimeric cytokine receptor of claim 46, wherein the hinge region derived from the IL-4 receptor alpha chain comprises the amino acid sequence of SEQ ID NO: 74, or a sequence having at least 80% identity thereof.

69. The chimeric cytokine receptor of claim 46, wherein the transmembrane region derived from the IL-4 receptor alpha chain comprises the amino acid sequence of SEQ ID NO: 76, or a sequence having at least 80% identity thereof.

70. The chimeric cytokine receptor of any one of claims 17 and 47, wherein intracellular signaling region derived from the IL-12 receptor beta 1 chain comprises the amino acid sequence of SEQ ID NO: 88, or an amino acid sequence having at least 80% identity thereof.

71. The chimeric cytokine receptor of claim 47, wherein the hinge region derived from the IL-12 receptor beta 1 chain comprises the amino acid sequence of SEQ ID NO: 84, or a sequence having at least 80% identity thereof.

72. The chimeric cytokine receptor of claim 47, wherein the transmembrane region derived from the IL-12 receptor beta 1 chain comprises the amino acid sequence of SEQ ID NO: 86, or a sequence having at least 80% identity thereof.

73. The chimeric cytokine receptor of any one of claims 17, 18, 47 and 48, wherein intracellular signaling region derived from the IL-12 receptor beta 2 chain comprises the amino acid sequence of SEQ ID NO: 98, or an amino acid sequence having at least 80% identity thereof.

74. The chimeric cytokine receptor of claim 47 or 48, wherein the hinge region derived from the IL-12 receptor beta 2 chain comprises the amino acid sequence of SEQ ID NO: 94, or a sequence having at least 80% identity thereof.

75. The chimeric cytokine receptor of claim 47 or 48, wherein the transmembrane region derived from the IL-12 receptor beta 2 chain comprises the amino acid sequence of SEQ ID NO: 96, or a sequence having at least 80% identity thereof.

76. The chimeric cytokine receptor of any one of claim 18 or 48, wherein the intracellular signaling region derived from the IL-23 receptor chain comprises the amino acid sequence of SEQ ID NO: 108, or an amino acid sequence having at least 80% identity thereof.

77. The chimeric cytokine receptor of claim 48, wherein the hinge region derived from the IL-23 receptor chain comprises the amino acid sequence of SEQ ID NO: 104, or a sequence having at least 80% identity thereof.

78. The chimeric cytokine receptor of claim 48, wherein the transmembrane region derived from the IL-23 receptor chain comprises the amino acid sequence of SEQ ID NO: 106, or a sequence having at least 80% identity thereof.

79. The chimeric cytokine receptor of any one of claim 19 or 49, wherein intracellular signaling region derived from the IL-10 receptor alpha chain comprises the amino acid sequence of SEQ ID NO: 118, or an amino acid sequence having at least 80% identity thereof.

80. The chimeric cytokine receptor of claim 49, wherein the hinge region derived from the IL-10 receptor alpha chain comprises the amino acid sequence of SEQ ID NO: 114, or a sequence having at least 80% identity thereof.

81. The chimeric cytokine receptor of claim 49, wherein the transmembrane region derived from the IL-10 receptor alpha chain comprises the amino acid sequence of SEQ ID NO: 116, or a sequence having at least 80% identity thereof.

82. The chimeric cytokine receptor of any one of claim 19 or 49, wherein intracellular signaling region derived from the IL-10 receptor beta chain comprises the amino acid sequence of SEQ ID NO: 128, or an amino acid sequence having at least 80% identity thereof.

83. The chimeric cytokine receptor of claim 49, wherein the hinge region derived from the IL-10 receptor beta chain comprises the amino acid sequence of SEQ ID NO: 124, or a sequence having at least 80% identity thereof.

84. The chimeric cytokine receptor of claim 49, wherein the transmembrane region derived from the IL-10 receptor beta chain comprises the amino acid sequence of SEQ ID NO: 126, or a sequence having at least 80% identity thereof.

85. The chimeric cytokine receptor of any one of claim 20 or 50, wherein intracellular signaling region derived from gp130 comprises the amino acid sequence of SEQ ID NO: 138, or an amino acid sequence having at least 80% identity thereof.

86. The chimeric cytokine receptor of claim 50, wherein the hinge region derived from gp130 comprises the amino acid sequence of SEQ ID NO: 134, or a sequence having at least 80% identity thereof.

87. The chimeric cytokine receptor of claim 50, wherein the transmembrane region derived from gp130 comprises the amino acid sequence of SEQ ID NO: 136, or a sequence having at least 80% identity thereof.

88. The chimeric cytokine receptor of claim 42, wherein the first polypeptide comprises the amino acid sequence of SEQ ID NO: 42, or a sequence having at least 80% identity thereof; and/or the second polypeptide comprises the amino acid sequence of SEQ ID NO: 40, or a sequence having at least 80% identity thereof.

89. The chimeric cytokine receptor of claim 43, wherein the first polypeptide comprises the amino acid sequence of SEQ ID NO: 42, or a sequence having at least 80% identity thereof; and/or the second polypeptide comprises the amino acid sequence of SEQ ID NO: 52, or a sequence having at least 80% identity thereof.

90. The chimeric cytokine receptor of claim 44, wherein the first polypeptide comprises the amino acid sequence of SEQ ID NO: 42, or a sequence having at least 80% identity thereof; and/or the second polypeptide comprises the amino acid sequence of SEQ ID NO: 62, or a sequence having at least 80% identity thereof.

91. The chimeric cytokine receptor of claim 45, wherein the first polypeptide comprises the amino acid sequence of SEQ ID NO: 42, or a sequence having at least 80% identity thereof; and/or the second polypeptide comprises the amino acid sequence of SEQ ID NO: 72, or a sequence having at least 80% identity thereof.

92. The chimeric cytokine receptor of claim 46, wherein the first polypeptide comprises the amino acid sequence of SEQ ID NO: 42, or a sequence having at least 80% identity thereof; and/or the second polypeptide comprises the amino acid sequence of SEQ ID NO: 82, or a sequence having at least 80% identity thereof.

93. The chimeric cytokine receptor of claim 47, wherein the first polypeptide comprises the amino acid sequence of SEQ ID NO: 92, or a sequence having at least 80% identity thereof; and/or the second polypeptide comprises the amino acid sequence of SEQ ID NO: 102, or a sequence having at least 80% identity thereof.

94. The chimeric cytokine receptor of claim 48, wherein the first polypeptide comprises the amino acid sequence of SEQ ID NO: 112, or a sequence having at least 80% identity thereof, and/or the second polypeptide comprises the amino acid sequence of SEQ ID NO: 102, or a sequence having at least 80% identity thereof.

95. The chimeric cytokine receptor of claim 49, wherein the first polypeptide comprises the amino acid sequence of SEQ ID NO: 122, or a sequence having at least 80% identity thereof, and/or the second polypeptide comprises the amino acid sequence of SEQ ID NO: 132, or a sequence having at least 80% identity thereof.

96. The chimeric cytokine receptor of claim 50, wherein the first polypeptide comprises the amino acid sequence of SEQ ID NO: 142, or a sequence having at least 80% identity thereof, and/or the second polypeptide comprises the amino acid sequence of SEQ ID NO: 152, or a sequence having at least 80% identity thereof.

97. The chimeric cytokine receptor of any one of claims 1-96, wherein the first and/or the second polypeptide further comprises a leader sequence.

98. The chimeric cytokine receptor of claim 97, wherein the leader sequence is derived from an immunoglobulin heavy chain variable region or colony stimulating factor 2 receptor alpha chain (CSF2RA).

99. The chimeric cytokine receptor of claim 98, wherein the leader sequence derived from an immunoglobulin heavy chain variable region comprises the amino acid sequence
(SEQ ID NO: 3)
MDWIWRILFLVGAATGAHS.

100. The chimeric cytokine receptor of any one of claims 97-99, wherein the first and second leader sequences are the same.

101. The chimeric cytokine receptor of any one of claims 97-99, wherein the first and second leader sequences are different.

102. The chimeric cytokine receptor of any one of claims 1-101, wherein the first and/or second polypeptide further comprises one or more additional polypeptide sequences.

103. The chimeric cytokine receptor of claim 102, wherein the one or more additional polypeptide sequences comprise are selected from one or more cellular markers, epitope tags, cytokines, safety switches, dimerization moieties, or degradation moieties.

104. A polynucleotide encoding the chimeric cytokine receptor of any one of claims 1-103.

105. The polynucleotide of claim 104, comprising
a) a nucleotide sequence encoding the first polypeptide of the chimeric cytokine receptor; and
b) a nucleotide sequence encoding the second polypeptide of the chimeric cytokine receptor.

106. The polynucleotide of claim 105, wherein the nucleotide sequence encoding the first polypeptide of the chimeric cytokine receptor is operably linked to the first polypeptide of the chimeric cytokine receptor via a sequence encoding a self-cleaving peptide and/or an internal ribosomal entry site (IRES).

107. The polynucleotide of claim 106, wherein the self-cleaving peptide is a 2A peptide.

108. The polynucleotide of claim 107, wherein the 2A peptide is T2A, P2A, E2A, or F2A peptide.

109. The polynucleotide of claim 107 or 108, wherein the 2A peptide is a P2A peptide.

110. The polynucleotide of claim 109, wherein the P2A peptide comprises the amino acid sequence GSGATNFSLLKQAGDVEENPGP (SEQ ID NO: 22), or an amino acid sequence having at least 80% sequence identity thereof.

111. A polynucleotide comprising a nucleotide sequence encoding the first polypeptide of the chimeric cytokine receptor of any one of claims 1-102.

112. A polynucleotide comprising a nucleotide sequence encoding the second polypeptide of the chimeric cytokine receptor of any one of claims 1-102.

113. The polynucleotide of any one of claims 105-112, wherein the nucleotide sequence(s) is expressed in an inducible fashion, achieved with an inducible promoter, an inducible expression system, an artificial signaling circuit, and/or drug induced splicing.

114. The polynucleotide of any one of claims 105-110, wherein the nucleotide sequences encoding the first and second polypeptides of the chimeric cytokine receptor are operably linked to a single promoter.

115. The polynucleotide of any one of claims 105-110 and 111, wherein the nucleotide sequence encoding the first polypeptide of the chimeric cytokine receptor is operably linked to a first promoter.

116. The polynucleotide of any one of claims 105-110 and 112, wherein the nucleotide sequence encoding the second polypeptide of the chimeric cytokine receptor is operably linked to a second promoter.

117. The polynucleotide of any one of claims 105-110, wherein the nucleotide sequence encoding the first polypeptide of the chimeric cytokine receptor is operably linked to a first promoter, the nucleotide sequence encoding the second polypeptide of the chimeric cytokine receptor is operably linked to a second promoter, and the first and second promoters are the same.

118. The polynucleotide of any one of claims 105-110, wherein the nucleotide sequence encoding the first polypeptide of the chimeric cytokine receptor is operably linked to a first promoter, the nucleotide sequence encoding the second polypeptide of the chimeric cytokine receptor is operably linked to a second promoter, and the first and second promoters are different.

119. The polynucleotide of any one of claims 114-118, wherein the promoter is an inducible promoter.

120. The polynucleotide of any one of claims 114-118, wherein the promoter is a T cell-specific promoter or an NK cell-specific promoter.

121. The polynucleotide of any one of claims 114-120, further comprising one or more additional nucleotide sequences encoding one or more additional polypeptide sequences.

122. The polynucleotide of claim 121, wherein the one or more additional polypeptide sequences are selected from one or more cellular markers, epitope tags, cytokines, safety switches, dimerization moieties, or degradation moieties.

123. The polynucleotide of claim 122, wherein the epitope tag is FLAG or Myc.

124. The polynucleotide of claim 122, wherein the cellular marker is mClover3 or mRuby.

125. The polynucleotide of any one of claims 104-124 which is a DNA molecule.

126. The polynucleotide of any one of claims 104-124 which is an RNA molecule.

127. A recombinant vector comprising the polynucleotide of any one of claims 104-126.

128. The recombinant vector of claim 127, wherein the vector is a viral vector.

129. The recombinant vector of claim 128, wherein the viral vector is a retroviral vector, a lentiviral vector, an adenoviral vector, an adeno-associated virus vector, an alphaviral vector, a herpes virus vector, a baculoviral vector, or a vaccinia virus vector.

130. The recombinant vector of claim 129, wherein the viral vector is a retroviral vector.

131. The recombinant vector of claim 127, wherein the vector is a non-viral vector.

132. The recombinant vector of claim 131, wherein the non-viral vector is a minicircle plasmid, a Sleeping Beauty transposon, a piggyBac transposon, or a single or double stranded DNA molecule that is used as a template for homology directed repair (HDR) based gene editing.

133. An isolated host cell comprising the polynucleotide of any one of claims 104-126 or the recombinant vector of any one of claims 127-132.

134. An isolated host cell comprising a chimeric cytokine receptor encoded by the polynucleotide of any one of claims 104-126.

135. The isolated host cell of claim 133 or claim 134, wherein the host cell is an immune cell.

136. The isolated host cell of any one of claims 133-135, wherein the host cell is a T cell, a natural killer (NK) cell, a mesenchymal stem cell (MSC), or a macrophage.

137. The isolated host cell of any one of claims 133-136, wherein the host cell is a T cell.

138. The isolated host cell of claim 137, wherein the host cell is an αβ T-cell receptor (TCR) T-cell, a γδ T-cell, a CD8+ T-cell, a CD4+ T-cell, a cytotoxic T-cell, an invariant natural killer T (iNKT) cell, a memory T-cell, a memory stem T-cell (T_{SCM}), a naïve T-cell, an effector T-cell, a T-helper cell, or a regulatory T-cell (Treg).

139. The isolated host cell of any one of claims 133-136, wherein the host cell is a NK cell derived from peripheral, cord blood, induced pluripotent stem (iPS) cells, and/or a cell line.

140. The isolated host cell of any one of claims 133-139, wherein the host cell further expresses one or more antigen-recognition molecules.

141. The isolated host cell of claim 140, wherein the one or more antigen-recognition molecules are selected from αβ T cell receptors (TCRs), synthetic T cell receptors and antigen receptor (STARs), chimeric antigen receptor (CARs), T cell antigen couplers (TACs), T cell receptor fusion constructs (TruCs), or antibodies, or a combination thereof.

142. The isolated host cell of any one of claims 133-141, wherein the host cell is further genetically modified to enhance its function by expressing one or more additional genes or deleting one or more inhibitory genes (e.g. REGNASE-1, DNMT3A) with a gene editing technology.

143. The isolated host cell of claim 142, wherein the one or more additional genes are selected from one or more transcription factors.

144. The isolated host cell of claim 143, wherein the transcription factor is c-Jun.

145. The isolated host cell of claim 142, wherein the one or more inhibitory genes are selected from REGNASE-1 and/or DNMT3A.

146. The isolated host cell of claim 142, wherein the gene editing technology is CRISPR-Cas9 or transcription activator-like effector nuclease (TALEN).

147. The isolated host cell of any one of claims 133-146, wherein the host cell has been activated and/or expanded ex vivo.

148. The isolated host cell of any one of claims 133-147, wherein the host cell is an allogeneic cell.

149. The isolated host cell of any one of claims 133-147, wherein the host cell is an autologous cell.

150. The isolated host cell of any one of claims 135-149, wherein the immune cells is derived from an induced pluripotent stem (iPS) cells.

151. A pharmaceutical composition comprising the host cell of any one of claims 133-150 and a pharmaceutically acceptable carrier and/or excipient.

152. A method of enhancing an effector function of an immune cell, wherein the immune cell expresses a chimeric antigen receptor (CAR), comprising genetically modifying the cell with the polynucleotide of any one of claims 104-126 or the recombinant vector of any one of claims 127-132.

153. The method of claim 152, wherein the effector function is one or more of expansion, persistence, and/or anti-tumor activity.

154. A method of generating the isolated host cell of any one of claims 133-150, said method comprising genetically modifying the host cell with the polynucleotide of any one of claims 104-126 or the recombinant vector of any one of claims 127-132.

155. The method of claim 154, further comprising genetically modifying the host cell to express a chimeric antigen receptor (CAR).

156. The method of claim 154 or 155, wherein the genetic modifying step is conducted via viral gene delivery.

157. The method of claim 154 or 155, wherein the genetic modifying step is conducted via non-viral gene delivery.

158. The method of any one of claims 154-157, wherein the genetically modifying step is conducted ex vivo.

159. The method of any one of claims 154-158, wherein the method further comprises activation and/or expansion of the host cell ex vivo before, after and/or during said genetic modification.

160. The method of any one of claims 154-159, wherein the host cell is an immune cell.

161. The method of any one of claims 152-160, wherein the immune cell is a T cell, a natural killer (NK) cell, a mesenchymal stem cell (MSC), or a macrophage.

162. The method of any one of claims 152-161, wherein the cell is a T cell.

163. The method of claim 162, wherein the cell is an αβ T-cell receptor (TCR) T-cell, a γδ T-cell, a CD8+ T-cell, a CD4+ T-cell, a cytotoxic T-cell, an invariant natural killer T (iNKT) cell, a memory T-cell, a memory stem T-cell (T_{SCM}), a naïve T-cell, an effector T-cell, a T-helper cell, or a regulatory T-cell (Treg).

164. The method of any one of claims 152-161, wherein the cell is a NK cell derived from peripheral, cord blood, iPS cells, and/or a cell line.

165. A method of treating a disease comprising administering to the subject an effective amount of the host cell of any one of claims 140-150, or the pharmaceutical composition of claim 151.

166. The method of any one of claim 165, said method comprising
a) isolating T cells or NK cells from the subject or donor;
b) modifying said T cells or NK cells ex vivo with the polynucleotide of any one of claims 104-126 or the recombinant vector of any one of claims 127-132;
c) optionally modifying said T cells or NK cells ex vivo to express a chimeric antigen receptor (CAR) that binds an antigen associated with said disease;
d) optionally, expanding and/or activating the modified T cells or NK cells before, after and/or during step b) or c); and
e) introducing a therapeutically effective amount of the modified T cells or NK cells into the subject.

167. The method of any one of claims 165-166, wherein the disease is a cancer, infection, or autoimmune disease.

168. The method of any one of claims 165-167, wherein the subject is human.


SEQUENCE LISTING (332 sequences)

SEQ ID NO: 1 - protein, 43 aa, synthetic construct
LEIEAAFLERENTALETRVAELRQRVQRLRNRVSQYRTRYGPL

SEQ ID NO: 2 - protein, 43 aa, synthetic construct
LEIRAAFLRQRNTALRTEVAELEQEVQRLENEVSQYETRYGPL

SEQ ID NO: 3 - protein, 19 aa, synthetic construct
MDWIWRILFLVGAATGAHS

SEQ ID NO: 4 - DNA, 57 bp, synthetic construct
  source 1..57: mol_type = other DNA
atggattggatctggcggatcctgttccttgtgggagctgccacaggcgcccacagc

SEQ ID NO: 5 - DNA, 57 bp, synthetic construct
  source 1..57: mol_type = other DNA
atggattggatctggcggatcctgttccttgtgggagctgccacaggcgcccactct

SEQ ID NO: 6 - protein, 8 aa, synthetic construct
DYKDDDDK

SEQ ID NO: 7 - DNA, 24 bp, synthetic construct
  source 1..24: mol_type = other DNA
gactacaaggacgacgacgacaag

SEQ ID NO: 8 - protein, 51 aa, synthetic construct
MDPDLEIEAAFLERENTALETRVAELRQRVQRLRNRVSQYRTRYGPLGGGK

SEQ ID NO: 9 - DNA, 153 bp, synthetic construct
  source 1..153: mol_type = other DNA
atggaccccgacctggaaatcgaggccgccttcctggaaagagagaacacagccctggaaaccagagtggccgagctgagacagagagtgcagcggctgagaaaccgggtgtcccagtacagaaccagatacggacctctcggcggaggaaaa

SEQ ID NO: 10 - DNA, 153 bp, synthetic construct
  source 1..153: mol_type = other DNA
atggatcctgacctcgagattgaagccgcctttctcgagcgcgagaataccgctctcgagacaagagtggctgaactgcggcagagggtccagagactgaggaacagagtgtctcagtaccggacacgctacggccctcttggaggtggaaaa

SEQ ID NO: 11 - protein, 15 aa, synthetic construct
GGGGSGGGGSGGGGS

SEQ ID NO: 12 - DNA, 45 bp, synthetic construct
  source 1..45: mol_type = other DNA
ggtggcggaggttctggcggaggcggctctggtggtggtggatct

SEQ ID NO: 13 - DNA, 45 bp, synthetic construct
  source 1..45: mol_type = other DNA
ggtggcggaggttctggcggaggcggaagtggtggtggcggatct

SEQ ID NO: 14 - protein, 15 aa, synthetic construct
PLAFRTKPAALGKDT

SEQ ID NO: 15 - DNA, 45 bp, synthetic construct
  source 1..45: mol_type = other DNA
cctctggccttcagaacaaaacctgccgctctgggcaaagacaca

SEQ ID NO: 16 - protein, 25 aa, synthetic construct
IPWLGHLLVGLSGAFGFIILVYLLI

SEQ ID NO: 17 - DNA, 75 bp, synthetic construct
  source 1..75: mol_type = other DNA
atcccctggctgggacatctgctcgttggactgtctggcgccttcggcttcatcatcctggtgtacctgctgatc

SEQ ID NO: 18 - protein, 286 aa, synthetic construct
NCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 19 - DNA, 859 bp, synthetic construct
  source 1..859: mol_type = other DNA
aactgccggaacacaggcccttggctgaagaaagtgctgaagtgcaacacccctgatccgagcaagttctttagccagctgagcagcgagcatggcggcgacgttcagaaatggctgtctagcccatttcctagcagcagcttcagccctggtggactggcccctgagattagccctctggaagtgctggaacgggacaaagtgacccagctgctcctgcagcaggataaggtgccagaacctgccagcctgtccagcaatcacagcctgaccagctgctttaccaaccagggctacttcttcttccatctgcctgacgctctcgagatcgaggcctgccaggtgtacttcacctacgatccctacagcgaggaagatcccgacgaaggcgttgcaggcgctcctacaggatcttctccacagcctctgcagccactgtccggcgaggatgatgcctactgcacctttccaagccgggacgacctgctgctgtttagccctagtctgctcggcggaccttctccaccatctacagctccaggtggaagcggagccggcgaagaaagaatgcctcctagcctgcaagagcgggtgcccagagattgggatcctcaacctctcggacctcctacaccaggcgtgccagacctggtggattttcagcctcctccagagctggtgctgagagaagctggcgaagaagtgccagacgccggacctagagagggcgttagctttccttggagcagacctcctggacagggcgagttcagagccctgaatgctagactgcccctgaacaccgatgcctacctgtctctgcaagaactgcagggacaagaccccacacacctcgttg

SEQ ID NO: 20 - protein, 326 aa, synthetic construct
PLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 21 - DNA, 979 bp, synthetic construct
  source 1..979: mol_type = other DNA
cctctggccttcagaacaaaacctgccgctctgggcaaagacacaatcccctggctgggacatctgctcgttggactgtctggcgccttcggcttcatcatcctggtgtacctgctgatcaactgccggaacacaggcccttggctgaagaaagtgctgaagtgcaacacccctgatccgagcaagttctttagccagctgagcagcgagcatggcggcgacgttcagaaatggctgtctagcccatttcctagcagcagcttcagccctggtggactggcccctgagattagccctctggaagtgctggaacgggacaaagtgacccagctgctcctgcagcaggataaggtgccagaacctgccagcctgtccagcaatcacagcctgaccagctgctttaccaaccagggctacttcttcttccatctgcctgacgctctcgagatcgaggcctgccaggtgtacttcacctacgatccctacagcgaggaagatcccgacgaaggcgttgcaggcgctcctacaggatcttctccacagcctctgcagccactgtccggcgaggatgatgcctactgcacctttccaagccgggacgacctgctgctgtttagccctagtctgctcggcggaccttctccaccatctacagctccaggtggaagcggagccggcgaagaaagaatgcctcctagcctgcaagagcgggtgcccagagattgggatcctcaacctctcggacctcctacaccaggcgtgccagacctggtggattttcagcctcctccagagctggtgctgagagaagctggcgaagaagtgccagacgccggacctagagagggcgttagctttccttggagcagacctcctggacagggcgagttcagagccctgaatgctagactgcccctgaacaccgatgcctacctgtctctgcaagaactgcagggacaagaccccacacacctcgttg

SEQ ID NO: 22 - protein, 22 aa, unidentified
GSGATNFSLLKQAGDVEENPGP

SEQ ID NO: 23 - DNA, 66 bp, unidentified
  source 1..66: mol_type = unassigned DNA
ggtagcggcgccacaaatttcagcctgctgaaacaggccggcgacgtggaagagaaccctggacct

SEQ ID NO: 24 - DNA, 66 bp, unidentified
  source 1..66: mol_type = unassigned DNA
ggcagcggcgccaccaactttagcctgctgaaacaagccggcgacgtggaagagaatcccggacct

SEQ ID NO: 25 - protein, 10 aa, synthetic construct
EQKLISEEDL

SEQ ID NO: 26 - DNA, 30 bp, synthetic construct
  source 1..30: mol_type = other DNA
gagcagaagctgatctccgaagaggacctg

SEQ ID NO: 27 - protein, 51 aa, synthetic construct
MDPDLEIRAAFLRQRNTALRTEVAELEQEVQRLENEVSQYETRYGPLGGGK

SEQ ID NO: 28 - DNA, 153 bp, synthetic construct
  source 1..153: mol_type = other DNA
atggaccccgacctggaaatcagagccgccttcctgcggcagagaaacacagccctgagaacagaggtggccgagctggaacaagaggtgcagcggctggaaaacgaggtgtcccagtacgagacaagatacggccctctcggcggaggaaaa

SEQ ID NO: 29 - DNA, 153 bp, synthetic construct
  source 1..153: mol_type = other DNA
atggatcctgacctcgagattagggccgcctttctgagacagcggaacaccgctctgaggaccgaagtggccgaactcgagcaagaagtccagagactggaaaatgaagtctctcagtacgaaacccgctacggacctcttggaggcggcaaa

SEQ ID NO: 30 - protein, 15 aa, synthetic construct
SNTSKENPFLFALEA

SEQ ID NO: 31 - DNA, 45 bp, synthetic construct
  source 1..45: mol_type = other DNA
agcaacaccagcaaagagaaccccttcctgttcgccctggaagcc

SEQ ID NO: 32 - protein, 21 aa, synthetic construct
VVISVGSMGLIISLLCVYFWL

SEQ ID NO: 33 - DNA, 63 bp, synthetic construct
  source 1..63: mol_type = other DNA
gtggttatcagcgtgggctctatgggcctgatcatcagcctgctgtgcgtgtacttctggctg

SEQ ID NO: 34 - protein, 86 aa, synthetic construct
ERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 35 - DNA, 258 bp, synthetic construct
  source 1..258: mol_type = other DNA
gaacggaccatgcctcggatccccacactgaagaacctggaagatctggtcaccgagtaccacggcaacttctctgcttggagcggcgtgtcaaaaggcctggccgaatctctgcagcccgactactctgagagactgtgcctggtgtctgagatccctcctaaaggcggagccctcggagaaggacctggtgcctctccatgtaaccagcacagcccttattgggcccctccttgctacaccctgaagcctgaaaca

SEQ ID NO: 36 - protein, 122 aa, synthetic construct
SNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 37 - DNA, 366 bp, synthetic construct
  source 1..366: mol_type = other DNA
agcaacaccagcaaagagaaccccttcctgttcgccctggaagccgtggttatcagcgtgggctctatgggcctgatcatcagcctgctgtgcgtgtacttctggctggaacggaccatgcctcggatccccacactgaagaacctggaagatctggtcaccgagtaccacggcaacttctctgcttggagcggcgtgtcaaaaggcctggccgaatctctgcagcccgactactctgagagactgtgcctggtgtctgagatccctcctaaaggcggagccctcggagaaggacctggtgcctctccatgtaaccagcacagcccttattgggcccctccttgctacaccctgaagcctgaaaca

SEQ ID NO: 38 - protein, 239 aa, synthetic construct
MVSKGEELFTGVVPILVELDGDVNGHKFSVRGEGEGDATNGKLTLKFICTTGKLPVPWPTLVTTFGYGVACFSRYPDHMKQHDFFKSAMPEGYVQERTISFKDDGTYKTRAEVKFEGDTLVNRIELKGIDFKEDGNILGHKLEYNFNSHYVYITADKQKNCIKANFKIRHNVEDGSVQLADHYQQNTPIGDGPVLLPDNHYLSHQSKLSKDPNEKRDHMVLLEFVTAAGITHGMDELYK

SEQ ID NO: 39 - DNA, 720 bp, synthetic construct
  source 1..720: mol_type = other DNA
atggtgagcaagggcgaggagctgttcaccggggtggtgcccatcctggtcgagctggacggcgacgtaaacggccacaagttcagcgtccgcggcgaaggtgagggagatgccaccaacggcaagctgaccctgaagttcatctgcaccaccggcaagctgcccgtgccctggcccaccctcgtgaccaccttcggctacggcgtggcctgcttcagccgctaccccgaccacatgaagcagcacgacttcttcaagtccgccatgcccgaaggctacgtccaggagcgcaccatctctttcaaggacgacggtacctacaagacccgcgccgaggtgaagttcgagggcgacaccctggtgaaccgcatcgagctgaagggcatcgacttcaaggaggacggcaacatcctggggcacaagctggagtacaacttcaacagccactacgtctatatcacggccgacaagcagaagaactgcatcaaggctaacttcaagatccgccacaacgttgaggacggcagcgtgcagctcgccgaccactaccagcagaacacccccatcggcgacggccccgtgctgctgcccgacaaccactacctgagccatcagtccaagctgagcaaagaccccaacgagaagcgcgatcacatggtcctgctggagttcgtgaccgccgccgggattacacatggcatggacgagctgtacaagtaa

SEQ ID NO: 40 - protein, 443 aa, synthetic construct
MDPDLEIEAAFLERENTALETRVAELRQRVQRLRNRVSQYRTRYGPLGGGKGGGGSGGGGSGGGGSMDPDLEIEAAFLERENTALETRVAELRQRVQRLRNRVSQYRTRYGPLGGGKPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 41 - DNA, 1330 bp, synthetic construct
  source 1..1330: mol_type = other DNA
atggaccccgacctggaaatcgaggccgccttcctggaaagagagaacacagccctggaaaccagagtggccgagctgagacagagagtgcagcggctgagaaaccgggtgtcccagtacagaaccagatacggacctctcggcggaggaaaaggtggcggaggttctggcggaggcggctctggtggtggtggatctatggatcctgacctcgagattgaagccgcctttctcgagcgcgagaataccgctctcgagacaagagtggctgaactgcggcagagggtccagagactgaggaacagagtgtctcagtaccggacacgctacggccctcttggaggtggaaaacctctggccttcagaacaaaacctgccgctctgggcaaagacacaatcccctggctgggacatctgctcgttggactgtctggcgccttcggcttcatcatcctggtgtacctgctgatcaactgccggaacacaggcccttggctgaagaaagtgctgaagtgcaacacccctgatccgagcaagttctttagccagctgagcagcgagcatggcggcgacgttcagaaatggctgtctagcccatttcctagcagcagcttcagccctggtggactggcccctgagattagccctctggaagtgctggaacgggacaaagtgacccagctgctcctgcagcaggataaggtgccagaacctgccagcctgtccagcaatcacagcctgaccagctgctttaccaaccagggctacttcttcttccatctgcctgacgctctcgagatcgaggcctgccaggtgtacttcacctacgatccctacagcgaggaagatcccgacgaaggcgttgcaggcgctcctacaggatcttctccacagcctctgcagccactgtccggcgaggatgatgcctactgcacctttccaagccgggacgacctgctgctgtttagccctagtctgctcggcggaccttctccaccatctacagctccaggtggaagcggagccggcgaagaaagaatgcctcctagcctgcaagagcgggtgcccagagattgggatcctcaacctctcggacctcctacaccaggcgtgccagacctggtggattttcagcctcctccagagctggtgctgagagaagctggcgaagaagtgccagacgccggacctagagagggcgttagctttccttggagcagacctcctggacagggcgagttcagagccctgaatgctagactgcccctgaacaccgatgcctacctgtctctgcaagaactgcagggacaagaccccacacacctcgttg

SEQ ID NO: 42 - protein, 239 aa, synthetic construct
MDPDLEIRAAFLRQRNTALRTEVAELEQEVQRLENEVSQYETRYGPLGGGKGGGGSGGGGSGGGGSMDPDLEIRAAFLRQRNTALRTEVAELEQEVQRLENEVSQYETRYGPLGGGKSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 43 - DNA, 717 bp, synthetic construct
  source 1..717: mol_type = other DNA
atggaccccgacctggaaatcagagccgccttcctgcggcagagaaacacagccctgagaacagaggtggccgagctggaacaagaggtgcagcggctggaaaacgaggtgtcccagtacgagacaagatacggccctctcggcggaggaaaaggtggcggaggttctggcggaggcggaagtggtggtggcggatctatggatcctgacctcgagattagggccgcctttctgagacagcggaacaccgctctgaggaccgaagtggccgaactcgagcaagaagtccagagactggaaaatgaagtctctcagtacgaaacccgctacggacctcttggaggcggcaaaagcaacaccagcaaagagaaccccttcctgttcgccctggaagccgtggttatcagcgtgggctctatgggcctgatcatcagcctgctgtgcgtgtacttctggctggaacggaccatgcctcggatccccacactgaagaacctggaagatctggtcaccgagtaccacggcaacttctctgcttggagcggcgtgtcaaaaggcctggccgaatctctgcagcccgactactctgagagactgtgcctggtgtctgagatccctcctaaaggcggagccctcggagaaggacctggtgcctctccatgtaaccagcacagcccttattgggcccctccttgctacaccctgaagcctgaaaca

SEQ ID NO: 44 - protein, 15 aa, synthetic construct
YFRTPEINNSSGEMD

SEQ ID NO: 45 - DNA, 45 bp, synthetic construct
  source 1..45: mol_type = other DNA
tacttcagaacccctgagatcaacaacagcagcggcgagatggat

SEQ ID NO: 46 - protein, 25 aa, synthetic construct
PILLTISILSFFSVALLVILACVLW

SEQ ID NO: 47 - DNA, 75 bp, synthetic construct
  source 1..75: mol_type = other DNA
cccatcctgctgacaatcagcatcctgagctttttcagcgtggccctgctggtcatcctggcctgtgtgctgtgg

SEQ ID NO: 48 - protein, 195 aa, synthetic construct
KKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 49 - DNA, 586 bp, synthetic construct
  source 1..586: mol_type = other DNA
aagaagcggatcaagcccatcgtgtggcccagcctgcctgaccacaagaaaaccctggaacacctgtgcaagaagccccggaagaacctgaacgtgtccttcaatcccgagagcttcctggactgtcagatccacagagtggacgacatccaggccagggacgaagtggaaggctttctgcaggacacattccctcagcagctggaagagagcgagaagcagagacttggaggcgacgtgcagagccctaattgcccttctgaggacgtggtcatcaccccagagagcttcggcagagatagcagcctgacatgtctggccggcaatgtgtccgcctgtgatgcccctatcctgagcagcagcagaagcctggattgcagagagagcggcaagaacggccctcacgtgtaccaggatctgctcctgtctctgggcaccaccaatagcacactgcctccaccattcagcctgcagagcggcatcctgacactgaaccctgttgctcagggccagccaatcctgacaagcctgggcagcaatcaagaagaggcctacgtcaccatgagcagcttctaccagaatcaag

SEQ ID NO: 50 - protein, 235 aa, synthetic construct
YFRTPEINNSSGEMDPILLTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 51 - DNA, 706 bp, synthetic construct
  source 1..706: mol_type = other DNA
tacttcagaacccctgagatcaacaacagcagcggcgagatggatcccatcctgctgacaatcagcatcctgagctttttcagcgtggccctgctggtcatcctggcctgtgtgctgtggaagaagcggatcaagcccatcgtgtggcccagcctgcctgaccacaagaaaaccctggaacacctgtgcaagaagccccggaagaacctgaacgtgtccttcaatcccgagagcttcctggactgtcagatccacagagtggacgacatccaggccagggacgaagtggaaggctttctgcaggacacattccctcagcagctggaagagagcgagaagcagagacttggaggcgacgtgcagagccctaattgcccttctgaggacgtggtcatcaccccagagagcttcggcagagatagcagcctgacatgtctggccggcaatgtgtccgcctgtgatgcccctatcctgagcagcagcagaagcctggattgcagagagagcggcaagaacggccctcacgtgtaccaggatctgctcctgtctctgggcaccaccaatagcacactgcctccaccattcagcctgcagagcggcatcctgacactgaaccctgttgctcagggccagccaatcctgacaagcctgggcagcaatcaagaagaggcctacgtcaccatgagcagcttctaccagaatcaag

SEQ ID NO: 52 - protein, 352 aa, synthetic construct
MDPDLEIEAAFLERENTALETRVAELRQRVQRLRNRVSQYRTRYGPLGGGKGGGGSGGGGSGGGGSMDPDLEIEAAFLERENTALETRVAELRQRVQRLRNRVSQYRTRYGPLGGGKYFRTPEINNSSGEMDPILLTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 53 - DNA, 1057 bp, synthetic construct
  source 1..1057: mol_type = other DNA
atggaccccgacctggaaatcgaggccgccttcctggaaagagagaacacagccctggaaaccagagtggccgagctgagacagagagtgcagcggctgagaaaccgggtgtcccagtacagaaccagatacggacctctcggcggaggaaaaggtggcggaggttctggcggaggcggctctggtggtggtggatctatggatcctgacctcgagattgaagccgcctttctcgagcgcgagaataccgctctcgagacaagagtggctgaactgcggcagagggtccagagactgaggaacagagtgtctcagtaccggacacgctacggccctcttggaggtggaaaatacttcagaacccctgagatcaacaacagcagcggcgagatggatcccatcctgctgacaatcagcatcctgagctttttcagcgtggccctgctggtcatcctggcctgtgtgctgtggaagaagcggatcaagcccatcgtgtggcccagcctgcctgaccacaagaaaaccctggaacacctgtgcaagaagccccggaagaacctgaacgtgtccttcaatcccgagagcttcctggactgtcagatccacagagtggacgacatccaggccagggacgaagtggaaggctttctgcaggacacattccctcagcagctggaagagagcgagaagcagagacttggaggcgacgtgcagagccctaattgcccttctgaggacgtggtcatcaccccagagagcttcggcagagatagcagcctgacatgtctggccggcaatgtgtccgcctgtgatgcccctatcctgagcagcagcagaagcctggattgcagagagagcggcaagaacggccctcacgtgtaccaggatctgctcctgtctctgggcaccaccaatagcacactgcctccaccattcagcctgcagagcggcatcctgacactgaaccctgttgctcagggccagccaatcctgacaagcctgggcagcaatcaagaagaggcctacgtcaccatgagcagcttctaccagaatcaag

SEQ ID NO: 54 - protein, 15 aa, synthetic construct
SDPVIFQTQSEELKE

SEQ ID NO: 55 - DNA, 45 bp, synthetic construct
  source 1..45: mol_type = other DNA
agcgaccccgtgatctttcagacccagagcgaggaactgaaagaa

SEQ ID NO: 56 - protein, 21 aa, synthetic construct
GWNPHLLLLLLLVIVFIPAFW

SEQ ID NO: 57 - DNA, 63 bp, synthetic construct
  source 1..63: mol_type = other DNA
ggctggaaccctcacctcctgctgttgctgctgctggtcatcgtgttcatccccgccttttgg

SEQ ID NO: 58 - protein, 285 aa, synthetic construct
SLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 59 - DNA, 855 bp, synthetic construct
  source 1..855: mol_type = other DNA
agcctgaaaacacaccctctgtggcggctgtggaagaaaatctgggccgtgccatctcctgagcggttcttcatgcctctgtacaagggctgcagcggcgacttcaagaaatgggtcggagccccttttaccggcagctctctggaacttggaccttggagccctgaggtgcccagcacactggaagtgtacagctgtcaccctcctagaagccccgccaagagactgcagctgacagagctgcaagagcctgccgagctggtggaatctgatggcgtgcccaagcctagcttctggcccacagctcagaatagcggcggctctgcctacagcgaggaaagagatagaccctacggcctggtgtccatcgacaccgtgacagtgctggatgccgagggaccttgtacctggccttgtagctgcgaggacgatggctaccctgctctggatctggacgctggccttgagccttctccaggactggaagatcctctgctggacgccggaacaaccgtgctgtcttgtggctgtgtgtctgccggatctcctggacttggaggccctctgggaagcctgctggatagactgaaacctcctctggccgacggcgaagattgggctggtggacttccttggggcggaagatctccaggcggagtgtctgagtctgaagccggttctccactggccggcctggacatggataccttcgattctggcttcgtgggcagcgactgtagcagccctgtggaatgcgacttcacaagccctggcgacgagggcccacctagaagctatctgagacagtgggtcgtgatccctccacctctgtctagtcctggacctcaggcttct

SEQ ID NO: 60 - protein, 321 aa, synthetic construct
SDPVIFQTQSEELKEGWNPHLLLLLLLVIVFIPAFWSLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 61 - DNA, 963 bp, synthetic construct
  source 1..963: mol_type = other DNA
agcgaccccgtgatctttcagacccagagcgaggaactgaaagaaggctggaaccctcacctcctgctgttgctgctgctggtcatcgtgttcatccccgccttttggagcctgaaaacacaccctctgtggcggctgtggaagaaaatctgggccgtgccatctcctgagcggttcttcatgcctctgtacaagggctgcagcggcgacttcaagaaatgggtcggagccccttttaccggcagctctctggaacttggaccttggagccctgaggtgcccagcacactggaagtgtacagctgtcaccctcctagaagccccgccaagagactgcagctgacagagctgcaagagcctgccgagctggtggaatctgatggcgtgcccaagcctagcttctggcccacagctcagaatagcggcggctctgcctacagcgaggaaagagatagaccctacggcctggtgtccatcgacaccgtgacagtgctggatgccgagggaccttgtacctggccttgtagctgcgaggacgatggctaccctgctctggatctggacgctggccttgagccttctccaggactggaagatcctctgctggacgccggaacaaccgtgctgtcttgtggctgtgtgtctgccggatctcctggacttggaggccctctgggaagcctgctggatagactgaaacctcctctggccgacggcgaagattgggctggtggacttccttggggcggaagatctccaggcggagtgtctgagtctgaagccggttctccactggccggcctggacatggataccttcgattctggcttcgtgggcagcgactgtagcagccctgtggaatgcgacttcacaagccctggcgacgagggcccacctagaagctatctgagacagtgggtcgtgatccctccacctctgtctagtcctggacctcaggcttct

SEQ ID NO: 62 - protein, 438 aa, synthetic construct
MDPDLEIEAAFLERENTALETRVAELRQRVQRLRNRVSQYRTRYGPLGGGKGGGGSGGGGSGGGGSMDPDLEIEAAFLERENTALETRVAELRQRVQRLRNRVSQYRTRYGPLGGGKSDPVIFQTQSEELKEGWNPHLLLLLLLVIVFIPAFWSLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 63 - DNA, 1314 bp, synthetic construct
  source 1..1314: mol_type = other DNA
atggaccccgacctggaaatcgaggccgccttcctggaaagagagaacacagccctggaaaccagagtggccgagctgagacagagagtgcagcggctgagaaaccgggtgtcccagtacagaaccagatacggacctctcggcggaggaaaaggtggcggaggttctggcggaggcggctctggtggtggtggatctatggatcctgacctcgagattgaagccgcctttctcgagcgcgagaataccgctctcgagacaagagtggctgaactgcggcagagggtccagagactgaggaacagagtgtctcagtaccggacacgctacggccctcttggaggtggaaaaagcgaccccgtgatctttcagacccagagcgaggaactgaaagaaggctggaaccctcacctcctgctgttgctgctgctggtcatcgtgttcatccccgccttttggagcctgaaaacacaccctctgtggcggctgtggaagaaaatctgggccgtgccatctcctgagcggttcttcatgcctctgtacaagggctgcagcggcgacttcaagaaatgggtcggagccccttttaccggcagctctctggaacttggaccttggagccctgaggtgcccagcacactggaagtgtacagctgtcaccctcctagaagccccgccaagagactgcagctgacagagctgcaagagcctgccgagctggtggaatctgatggcgtgcccaagcctagcttctggcccacagctcagaatagcggcggctctgcctacagcgaggaaagagatagaccctacggcctggtgtccatcgacaccgtgacagtgctggatgccgagggaccttgtacctggccttgtagctgcgaggacgatggctaccctgctctggatctggacgctggccttgagccttctccaggactggaagatcctctgctggacgccggaacaaccgtgctgtcttgtggctgtgtgtctgccggatctcctggacttggaggccctctgggaagcctgctggatagactgaaacctcctctggccgacggcgaagattgggctggtggacttccttggggcggaagatctccaggcggagtgtctgagtctgaagccggttctccactggccggcctggacatggataccttcgattctggcttcgtgggcagcgactgtagcagccctgtggaatgcgacttcacaagccctggcgacgagggcccacctagaagctatctgagacagtgggtcgtgatccctccacctctgtctagtcctggacctcaggcttct

SEQ ID NO: 64 - protein, 15 aa, synthetic construct
APQRQGPLIPPWGWP

SEQ ID NO: 65 - DNA, 45 bp, synthetic construct
  source 1..45: mol_type = other DNA
gctcctcagagacagggacctctgattcctccttggggctggcct

SEQ ID NO: 66 - protein, 21 aa, synthetic construct
GNTLVAVSIFLLLTGPTYLLF

SEQ ID NO: 67 - DNA, 63 bp, synthetic construct
  source 1..63: mol_type = other DNA
ggaaataccctggtggccgtgtccatcttcctgctgctgaccggaccaacctacctgctgttc

SEQ ID NO: 68 - protein, 230 aa, synthetic construct
KLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTF

SEQ ID NO: 69 - DNA, 690 bp, synthetic construct
  source 1..690: mol_type = other DNA
aagctgagccccagagtgaagcggatcttctaccagaacgtgccctctcctgccatgttcttccagcctctgtacagcgtgcacaacggcaacttccagacatggatgggagcacatggtgccggcgtgctgctgtctcaagattgtgctggaacaccccagggcgctctggaaccttgtgtgcaagaagccaccgctctgctgacatgcggacctgccagaccttggaaaagcgtggccctggaagaggaacaagaaggccctggcacaagactgcccggcaatctgtctagcgaggatgtgctgcctgccggctgtactgaatggcgagttcagaccctggcctacctgcctcaagaagattgggcccctaccagcctgaccagacctgctcctcctgatagcgagggcagcagaagcagcagcagctccagctcctccaacaacaacaattactgtgccctgggctgctacggcggctggcatctttctgctctgcctggcaacacacagagcagcggccctattcctgctctggcctgtggcctgagctgtgatcaccagggactcgagacacagcagggcgttgcatgggttctcgccggccattgtcaaagacctggcctgcacgaagatctgcagggcatgctgctgcctagcgtgctgtctaaggccagaagctggaccttc

SEQ ID NO: 70 - protein, 266 aa, synthetic construct
APQRQGPLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTF

SEQ ID NO: 71 - DNA, 798 bp, synthetic construct
  source 1..798: mol_type = other DNA
gctcctcagagacagggacctctgattcctccttggggctggcctggaaataccctggtggccgtgtccatcttcctgctgctgaccggaccaacctacctgctgttcaagctgagccccagagtgaagcggatcttctaccagaacgtgccctctcctgccatgttcttccagcctctgtacagcgtgcacaacggcaacttccagacatggatgggagcacatggtgccggcgtgctgctgtctcaagattgtgctggaacaccccagggcgctctggaaccttgtgtgcaagaagccaccgctctgctgacatgcggacctgccagaccttggaaaagcgtggccctggaagaggaacaagaaggccctggcacaagactgcccggcaatctgtctagcgaggatgtgctgcctgccggctgtactgaatggcgagttcagaccctggcctacctgcctcaagaagattgggcccctaccagcctgaccagacctgctcctcctgatagcgagggcagcagaagcagcagcagctccagctcctccaacaacaacaattactgtgccctgggctgctacggcggctggcatctttctgctctgcctggcaacacacagagcagcggccctattcctgctctggcctgtggcctgagctgtgatcaccagggactcgagacacagcagggcgttgcatgggttctcgccggccattgtcaaagacctggcctgcacgaagatctgcagggcatgctgctgcctagcgtgctgtctaaggccagaagctggaccttc

SEQ ID NO: 72 - protein, 383 aa, synthetic construct
MDPDLEIEAAFLERENTALETRVAELRQRVQRLRNRVSQYRTRYGPLGGGKGGGGSGGGGSGGGGSMDPDLEIEAAFLERENTALETRVAELRQRVQRLRNRVSQYRTRYGPLGGGKAPQRQGPLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTF

SEQ ID NO: 73 - DNA, 1149 bp, synthetic construct
  source 1..1149: mol_type = other DNA
atggaccccgacctggaaatcgaggccgccttcctggaaagagagaacacagccctggaaaccagagtggccgagctgagacagagagtgcagcggctgagaaaccgggtgtcccagtacagaaccagatacggacctctcggcggaggaaaaggtggcggaggttctggcggaggcggctctggtggtggtggatctatggatcctgacctcgagattgaagccgcctttctcgagcgcgagaataccgctctcgagacaagagtggctgaactgcggcagagggtccagagactgaggaacagagtgtctcagtaccggacacgctacggccctcttggaggtggaaaagctcctcagagacagggacctctgattcctccttggggctggcctggaaataccctggtggccgtgtccatcttcctgctgctgaccggaccaacctacctgctgttcaagctgagccccagagtgaagcggatcttctaccagaacgtgccctctcctgccatgttcttccagcctctgtacagcgtgcacaacggcaacttccagacatggatgggagcacatggtgccggcgtgctgctgtctcaagattgtgctggaacaccccagggcgctctggaaccttgtgtgcaagaagccaccgctctgctgacatgcggacctgccagaccttggaaaagcgtggccctggaagaggaacaagaaggccctggcacaagactgcccggcaatctgtctagcgaggatgtgctgcctgccggctgtactgaatggcgagttcagaccctggcctacctgcctcaagaagattgggcccctaccagcctgaccagacctgctcctcctgatagcgagggcagcagaagcagcagcagctccagctcctccaacaacaacaattactgtgccctgggctgctacggcggctggcatctttctgctctgcctggcaacacacagagcagcggccctattcctgctctggcctgtggcctgagctgtgatcaccagggactcgagacacagcagggcgttgcatgggttctcgccggccattgtcaaagacctggcctgcacgaagatctgcagggcatgctgctgcctagcgtgctgtctaaggccagaagctggaccttc

SEQ ID NO: 74 - protein, 15 aa, synthetic construct
STKWHNSYREPFEQH

SEQ ID NO: 75 - DNA, 45 bp, synthetic construct
  source 1..45: mol_type = other DNA
agcaccaagtggcacaacagctacagggagcccttcgagcagcac

SEQ ID NO: 76 - protein, 24 aa, synthetic construct
LLLGVSVSCIVILAVCLLCYVSIT

SEQ ID NO: 77 - DNA, 72 bp, synthetic construct
  source 1..72: mol_type = other DNA
ctgctgctgggcgtgagcgtgagctgcatcgtgatcctggccgtgtgcctgctgtgctacgtgagcatcacc

SEQ ID NO: 78 - protein, 569 aa, synthetic construct
KIKKEWWDQIPNPARSRLVAIIIQDAQGSQWEKRSRGQEPAKCPHWKNCLTKLLPCFLEHNMKRDEDPHKAAKEMPFQGSGKSAWCPVEISKTVLWPESISVVRCVELFEAPVECEEEEEVEEEKGSFCASPESSRDDFQEGREGIVARLTESLFLDLLGEENGGFCQQDMGESCLLPPSGSTSAHMPWDEFPSAGPKEAPPWGKEQPLHLEPSPPASPTQSPDNLTCTETPLVIAGNPAYRSFSNSLSQSPCPRELGPDPLLARHLEEVEPEMPCVPQLSEPTTVPQPEPETWEQILRRNVLQHGAAAAPVSAPTSGYQEFVHAVEQGGTQASAVVGLGPPGEAGYKAFSSLLASSAVSPEKCGFGASSGEEGYKPFQDLIPGCPGDPAPVPVPLFTFGLDREPPRSPQSSHLPSSSPEHLGLEPGEKVEDMPKPPLPQEQATDPLVDSLGSGIVYSALTCHLCGHLKQCHGQEDGGQTPVMASPCCGCCCGDRSSPPTTPLRAPDPSPGGVPLEASLCPASLAPSGISEKSKSSSSFHPAPGNAQSSSQTPKIVNFVSVGPTYMRVS

SEQ ID NO: 79 - DNA, 1707 bp, synthetic construct
  source 1..1707: mol_type = other DNA
aagatcaagaaggagtggtgggaccagatccccaaccccgccaggagcaggctggtggccatcatcatccaggacgcccagggcagccagtgggagaagaggagcaggggccaggagcccgccaagtgcccccactggaagaactgcctgaccaagctgctgccctgcttcctggagcacaacatgaagagggacgaggacccccacaaggccgccaaggagatgcccttccagggcagcggcaagagcgcctggtgccccgtggagatcagcaagaccgtgctgtggcccgagagcatcagcgtggtgaggtgcgtggagctgttcgaggcccccgtggagtgcgaggaggaggaggaggtggaggaggagaagggcagcttctgcgccagccccgagagcagcagggacgacttccaggagggcagggagggcatcgtggccaggctgaccgagagcctgttcctggacctgctgggcgaggagaacggcggcttctgccagcaggacatgggcgagagctgcctgctgccccccagcggcagcaccagcgcccacatgccctgggacgagttccccagcgccggccccaaggaggcccccccctggggcaaggagcagcccctgcacctggagcccagcccccccgccagccccacccagagccccgacaacctgacctgcaccgagacccccctggtgatcgccggcaaccccgcctacaggagcttcagcaacagcctgagccagagcccctgccccagggagctgggccccgaccccctgctggccaggcacctggaggaggtggagcccgagatgccctgcgtgccccagctgagcgagcccaccaccgtgccccagcccgagcccgagacctgggagcagatcctgaggaggaacgtgctgcagcacggcgccgccgccgcccccgtgagcgcccccaccagcggctaccaggagttcgtgcacgccgtggagcagggcggcacccaggccagcgccgtggtgggcctgggcccccccggcgaggccggctacaaggccttcagcagcctgctggccagcagcgccgtgagccccgagaagtgcggcttcggcgccagcagcggcgaggagggctacaagcccttccaggacctgatccccggctgccccggcgaccccgcccccgtgcccgtgcccctgttcaccttcggcctggacagggagccccccaggagcccccagagcagccacctgcccagcagcagccccgagcacctgggcctggagcccggcgagaaggtggaggacatgcccaagccccccctgccccaggagcaggccaccgaccccctggtggacagcctgggcagcggcatcgtgtacagcgccctgacctgccacctgtgcggccacctgaagcagtgccacggccaggaggacggcggccagacccccgtgatggccagcccctgctgcggctgctgctgcggcgacaggagcagcccccccaccacccccctgagggcccccgaccccagccccggcggcgtgcccctggaggccagcctgtgccccgccagcctggcccccagcggcatcagcgagaagagcaagagcagcagcagcttccaccccgcccccggcaacgcccagagcagcagccagacccccaagatcgtgaacttcgtgagcgtgggccccacctacatgagggtgagc

SEQ ID NO: 80 - protein, 608 aa, synthetic construct
STKWHNSYREPFEQHLLLGVSVSCIVILAVCLLCYVSITKIKKEWWDQIPNPARSRLVAIIIQDAQGSQWEKRSRGQEPAKCPHWKNCLTKLLPCFLEHNMKRDEDPHKAAKEMPFQGSGKSAWCPVEISKTVLWPESISVVRCVELFEAPVECEEEEEVEEEKGSFCASPESSRDDFQEGREGIVARLTESLFLDLLGEENGGFCQQDMGESCLLPPSGSTSAHMPWDEFPSAGPKEAPPWGKEQPLHLEPSPPASPTQSPDNLTCTETPLVIAGNPAYRSFSNSLSQSPCPRELGPDPLLARHLEEVEPEMPCVPQLSEPTTVPQPEPETWEQILRRNVLQHGAAAAPVSAPTSGYQEFVHAVEQGGTQASAVVGLGPPGEAGYKAFSSLLASSAVSPEKCGFGASSGEEGYKPFQDLIPGCPGDPAPVPVPLFTFGLDREPPRSPQSSHLPSSSPEHLGLEPGEKVEDMPKPPLPQEQATDPLVDSLGSGIVYSALTCHLCGHLKQCHGQEDGGQTPVMASPCCGCCCGDRSSPPTTPLRAPDPSPGGVPLEASLCPASLAPSGISEKSKSSSSFHPAPGNAQSSSQTPKIVNFVSVGPTYMRVS

SEQ ID NO: 81 - DNA, 1824 bp, synthetic construct
  source 1..1824: mol_type = other DNA
agcaccaagtggcacaacagctacagggagcccttcgagcagcacctgctgctgggcgtgagcgtgagctgcatcgtgatcctggccgtgtgcctgctgtgctacgtgagcatcaccaagatcaagaaggagtggtgggaccagatccccaaccccgccaggagcaggctggtggccatcatcatccaggacgcccagggcagccagtgggagaagaggagcaggggccaggagcccgccaagtgcccccactggaagaactgcctgaccaagctgctgccctgcttcctggagcacaacatgaagagggacgaggacccccacaaggccgccaaggagatgcccttccagggcagcggcaagagcgcctggtgccccgtggagatcagcaagaccgtgctgtggcccgagagcatcagcgtggtgaggtgcgtggagctgttcgaggcccccgtggagtgcgaggaggaggaggaggtggaggaggagaagggcagcttctgcgccagccccgagagcagcagggacgacttccaggagggcagggagggcatcgtggccaggctgaccgagagcctgttcctggacctgctgggcgaggagaacggcggcttctgccagcaggacatgggcgagagctgcctgctgccccccagcggcagcaccagcgcccacatgccctgggacgagttccccagcgccggccccaaggaggcccccccctggggcaaggagcagcccctgcacctggagcccagcccccccgccagccccacccagagccccgacaacctgacctgcaccgagacccccctggtgatcgccggcaaccccgcctacaggagcttcagcaacagcctgagccagagcccctgccccagggagctgggccccgaccccctgctggccaggcacctggaggaggtggagcccgagatgccctgcgtgccccagctgagcgagcccaccaccgtgccccagcccgagcccgagacctgggagcagatcctgaggaggaacgtgctgcagcacggcgccgccgccgcccccgtgagcgcccccaccagcggctaccaggagttcgtgcacgccgtggagcagggcggcacccaggccagcgccgtggtgggcctgggcccccccggcgaggccggctacaaggccttcagcagcctgctggccagcagcgccgtgagccccgagaagtgcggcttcggcgccagcagcggcgaggagggctacaagcccttccaggacctgatccccggctgccccggcgaccccgcccccgtgcccgtgcccctgttcaccttcggcctggacagggagccccccaggagcccccagagcagccacctgcccagcagcagccccgagcacctgggcctggagcccggcgagaaggtggaggacatgcccaagccccccctgccccaggagcaggccaccgaccccctggtggacagcctgggcagcggcatcgtgtacagcgccctgacctgccacctgtgcggccacctgaagcagtgccacggccaggaggacggcggccagacccccgtgatggccagcccctgctgcggctgctgctgcggcgacaggagcagcccccccaccacccccctgagggcccccgaccccagccccggcggcgtgcccctggaggccagcctgtgccccgccagcctggcccccagcggcatcagcgagaagagcaagagcagcagcagcttccaccccgcccccggcaacgcccagagcagcagccagacccccaagatcgtgaacttcgtgagcgtgggccccacctacatgagggtgagc

SEQ ID NO: 82 - protein, 725 aa, synthetic construct
MDPDLEIEAAFLERENTALETRVAELRQRVQRLRNRVSQYRTRYGPLGGGKGGGGSGGGGSGGGGSMDPDLEIEAAFLERENTALETRVAELRQRVQRLRNRVSQYRTRYGPLGGGKSTKWHNSYREPFEQHLLLGVSVSCIVILAVCLLCYVSITKIKKEWWDQIPNPARSRLVAIIIQDAQGSQWEKRSRGQEPAKCPHWKNCLTKLLPCFLEHNMKRDEDPHKAAKEMPFQGSGKSAWCPVEISKTVLWPESISVVRCVELFEAPVECEEEEEVEEEKGSFCASPESSRDDFQEGREGIVARLTESLFLDLLGEENGGFCQQDMGESCLLPPSGSTSAHMPWDEFPSAGPKEAPPWGKEQPLHLEPSPPASPTQSPDNLTCTETPLVIAGNPAYRSFSNSLSQSPCPRELGPDPLLARHLEEVEPEMPCVPQLSEPTTVPQPEPETWEQILRRNVLQHGAAAAPVSAPTSGYQEFVHAVEQGGTQASAVVGLGPPGEAGYKAFSSLLASSAVSPEKCGFGASSGEEGYKPFQDLIPGCPGDPAPVPVPLFTFGLDREPPRSPQSSHLPSSSPEHLGLEPGEKVEDMPKPPLPQEQATDPLVDSLGSGIVYSALTCHLCGHLKQCHGQEDGGQTPVMASPCCGCCCGDRSSPPTTPLRAPDPSPGGVPLEASLCPASLAPSGISEKSKSSSSFHPAPGNAQSSSQTPKIVNFVSVGPTYMRVS

SEQ ID NO: 83 - DNA, 2175 bp, synthetic construct
  source 1..2175: mol_type = other DNA
atggaccccgacctggaaatcgaggccgccttcctggaaagagagaacacagccctggaaaccagagtggccgagctgagacagagagtgcagcggctgagaaaccgggtgtcccagtacagaaccagatacggacctctcggcggaggaaaaggtggcggaggttctggcggaggcggctctggtggtggtggatctatggatcctgacctcgagattgaagccgcctttctcgagcgcgagaataccgctctcgagacaagagtggctgaactgcggcagagggtccagagactgaggaacagagtgtctcagtaccggacacgctacggccctcttggaggtggaaaaagcaccaagtggcacaacagctacagggagcccttcgagcagcacctgctgctgggcgtgagcgtgagctgcatcgtgatcctggccgtgtgcctgctgtgctacgtgagcatcaccaagatcaagaaggagtggtgggaccagatccccaaccccgccaggagcaggctggtggccatcatcatccaggacgcccagggcagccagtgggagaagaggagcaggggccaggagcccgccaagtgcccccactggaagaactgcctgaccaagctgctgccctgcttcctggagcacaacatgaagagggacgaggacccccacaaggccgccaaggagatgcccttccagggcagcggcaagagcgcctggtgccccgtggagatcagcaagaccgtgctgtggcccgagagcatcagcgtggtgaggtgcgtggagctgttcgaggcccccgtggagtgcgaggaggaggaggaggtggaggaggagaagggcagcttctgcgccagccccgagagcagcagggacgacttccaggagggcagggagggcatcgtggccaggctgaccgagagcctgttcctggacctgctgggcgaggagaacggcggcttctgccagcaggacatgggcgagagctgcctgctgccccccagcggcagcaccagcgcccacatgccctgggacgagttccccagcgccggccccaaggaggcccccccctggggcaaggagcagcccctgcacctggagcccagcccccccgccagccccacccagagccccgacaacctgacctgcaccgagacccccctggtgatcgccggcaaccccgcctacaggagcttcagcaacagcctgagccagagcccctgccccagggagctgggccccgaccccctgctggccaggcacctggaggaggtggagcccgagatgccctgcgtgccccagctgagcgagcccaccaccgtgccccagcccgagcccgagacctgggagcagatcctgaggaggaacgtgctgcagcacggcgccgccgccgcccccgtgagcgcccccaccagcggctaccaggagttcgtgcacgccgtggagcagggcggcacccaggccagcgccgtggtgggcctgggcccccccggcgaggccggctacaaggccttcagcagcctgctggccagcagcgccgtgagccccgagaagtgcggcttcggcgccagcagcggcgaggagggctacaagcccttccaggacctgatccccggctgccccggcgaccccgcccccgtgcccgtgcccctgttcaccttcggcctggacagggagccccccaggagcccccagagcagccacctgcccagcagcagccccgagcacctgggcctggagcccggcgagaaggtggaggacatgcccaagccccccctgccccaggagcaggccaccgaccccctggtggacagcctgggcagcggcatcgtgtacagcgccctgacctgccacctgtgcggccacctgaagcagtgccacggccaggaggacggcggccagacccccgtgatggccagcccctgctgcggctgctgctgcggcgacaggagcagcccccccaccacccccctgagggcccccgaccccagccccggcggcgtgcccctggaggccagcctgtgccccgccagcctggcccccagcggcatcagcgagaagagcaagagcagcagcagcttccaccccgcccccggcaacgcccagagcagcagccagacccccaagatcgtgaacttcgtgagcgtgggccccacctacatgagggtgagc

SEQ ID NO: 84 - protein, 15 aa, synthetic construct
WSQPQRFSIEVQVSD

SEQ ID NO: 85 - DNA, 45 bp, synthetic construct
  source 1..45: mol_type = other DNA
tggagccagccccagaggttcagcatcgaggtgcaggtgagcgac

SEQ ID NO: 86 - protein, 25 aa, synthetic construct
WLIFFASLGSFLSILLVGVLGYLGL

SEQ ID NO: 87 - DNA, 75 bp, synthetic construct
  source 1..75: mol_type = other DNA
tggctgatcttcttcgccagcctgggcagcttcctgagcatcctgctggtgggcgtgctgggctacctgggcctg

SEQ ID NO: 88 - protein, 92 aa, synthetic construct
NRAARHLCPPLPTPCASSAIEFPGGKETWQWINPVDFQEEASLQEALVVEMSWDKGERTEPLEKTELPEGAPELALDTELSLEDGDRCKAKM

SEQ ID NO: 89 - DNA, 276 bp, synthetic construct
  source 1..276: mol_type = other DNA
aacagggccgccaggcacctgtgcccccccctgcccaccccctgcgccagcagcgccatcgagttccccggcggcaaggagacctggcagtggatcaaccccgtggacttccaggaggaggccagcctgcaggaggccctggtggtggagatgagctgggacaagggcgagaggaccgagcccctggagaagaccgagctgcccgagggcgcccccgagctggccctggacaccgagctgagcctggaggacggcgacaggtgcaaggccaagatg

SEQ ID NO: 90 - protein, 132 aa, synthetic construct
WSQPQRFSIEVQVSDWLIFFASLGSFLSILLVGVLGYLGLNRAARHLCPPLPTPCASSAIEFPGGKETWQWINPVDFQEEASLQEALVVEMSWDKGERTEPLEKTELPEGAPELALDTELSLEDGDRCKAKM

SEQ ID NO: 91 - DNA, 396 bp, synthetic construct
  source 1..396: mol_type = other DNA
tggagccagccccagaggttcagcatcgaggtgcaggtgagcgactggctgatcttcttcgccagcctgggcagcttcctgagcatcctgctggtgggcgtgctgggctacctgggcctgaacagggccgccaggcacctgtgcccccccctgcccaccccctgcgccagcagcgccatcgagttccccggcggcaaggagacctggcagtggatcaaccccgtggacttccaggaggaggccagcctgcaggaggccctggtggtggagatgagctgggacaagggcgagaggaccgagcccctggagaagaccgagctgcccgagggcgcccccgagctggccctggacaccgagctgagcctggaggacggcgacaggtgcaaggccaagatg

SEQ ID NO: 92 - protein, 249 aa, synthetic construct
MDPDLEIEAAFLERENTALETRVAELRQRVQRLRNRVSQYRTRYGPLGGGKGGGGSGGGGSGGGGSMDPDLEIEAAFLERENTALETRVAELRQRVQRLRNRVSQYRTRYGPLGGGKWSQPQRFSIEVQVSDWLIFFASLGSFLSILLVGVLGYLGLNRAARHLCPPLPTPCASSAIEFPGGKETWQWINPVDFQEEASLQEALVVEMSWDKGERTEPLEKTELPEGAPELALDTELSLEDGDRCKAKM

SEQ ID NO: 93 - DNA, 747 bp, synthetic construct
  source 1..747: mol_type = other DNA
atggaccccgacctggaaatcgaggccgccttcctggaaagagagaacacagccctggaaaccagagtggccgagctgagacagagagtgcagcggctgagaaaccgggtgtcccagtacagaaccagatacggacctctcggcggaggaaaaggtggcggaggttctggcggaggcggctctggtggtggtggatctatggatcctgacctcgagattgaagccgcctttctcgagcgcgagaataccgctctcgagacaagagtggctgaactgcggcagagggtccagagactgaggaacagagtgtctcagtaccggacacgctacggccctcttggaggtggaaaatggagccagccccagaggttcagcatcgaggtgcaggtgagcgactggctgatcttcttcgccagcctgggcagcttcctgagcatcctgctggtgggcgtgctgggctacctgggcctgaacagggccgccaggcacctgtgcccccccctgcccaccccctgcgccagcagcgccatcgagttccccggcggcaaggagacctggcagtggatcaaccccgtggacttccaggaggaggccagcctgcaggaggccctggtggtggagatgagctgggacaagggcgagaggaccgagcccctggagaagaccgagctgcccgagggcgcccccgagctggccctggacaccgagctgagcctggaggacggcgacaggtgcaaggccaagatg

SEQ ID NO: 94 - protein, 15 aa, synthetic construct
SHGNEREFCLQGKAN

SEQ ID NO: 95 - DNA, 45 bp, synthetic construct
  source 1..45: mol_type = other DNA
agccacggcaacgagagggagttctgcctgcagggcaaggccaac

SEQ ID NO: 96 - protein, 21 aa, synthetic construct
WMAFVAPSICIAIIMVGIFST

SEQ ID NO: 97 - DNA, 63 bp, synthetic construct
  source 1..63: mol_type = other DNA
tggatggccttcgtggcccccagcatctgcatcgccatcatcatggtgggcatcttcagcacc

SEQ ID NO: 98 - protein, 219 aa, synthetic construct
HYFQQKVFVLLAALRPQWCSREIPDPANSTCAKKYPIAEEKTQLPLDRLLIDWPTPEDPEPLVISEVLHQVTPVFRHPPCSNWPQREKGIQGHQASEKDMMHSASSPPPPRALQAESRQLVDLYKVLESRGSDPKPENPACPWTVLPAGDLPTHDGYLPSNIDDLPSHEAPLADSLEELEPQHISLSVFPSSSLHPLTFSCGDKLTLDQLKMRCDSLML

SEQ ID NO: 99 - DNA, 657 bp, synthetic construct
  source 1..657: mol_type = other DNA
cactacttccagcagaaggtgttcgtgctgctggccgccctgaggccccagtggtgcagcagggagatccccgaccccgccaacagcacctgcgccaagaagtaccccatcgccgaggagaagacccagctgcccctggacaggctgctgatcgactggcccacccccgaggaccccgagcccctggtgatcagcgaggtgctgcaccaggtgacccccgtgttcaggcaccccccctgcagcaactggccccagagggagaagggcatccagggccaccaggccagcgagaaggacatgatgcacagcgccagcagcccccccccccccagggccctgcaggccgagagcaggcagctggtggacctgtacaaggtgctggagagcaggggcagcgaccccaagcccgagaaccccgcctgcccctggaccgtgctgcccgccggcgacctgcccacccacgacggctacctgcccagcaacatcgacgacctgcccagccacgaggcccccctggccgacagcctggaggagctggagccccagcacatcagcctgagcgtgttccccagcagcagcctgcaccccctgaccttcagctgcggcgacaagctgaccctggaccagctgaagatgaggtgcgacagcctgatgctg

SEQ ID NO: 100 - protein, 255 aa, synthetic construct
SHGNEREFCLQGKANWMAFVAPSICIAIIMVGIFSTHYFQQKVFVLLAALRPQWCSREIPDPANSTCAKKYPIAEEKTQLPLDRLLIDWPTPEDPEPLVISEVLHQVTPVFRHPPCSNWPQREKGIQGHQASEKDMMHSASSPPPPRALQAESRQLVDLYKVLESRGSDPKPENPACPWTVLPAGDLPTHDGYLPSNIDDLPSHEAPLADSLEELEPQHISLSVFPSSSLHPLTFSCGDKLTLDQLKMRCDSLML

SEQ ID NO: 101 - DNA, 765 bp, synthetic construct
  source 1..765: mol_type = other DNA
agccacggcaacgagagggagttctgcctgcagggcaaggccaactggatggccttcgtggcccccagcatctgcatcgccatcatcatggtgggcatcttcagcacccactacttccagcagaaggtgttcgtgctgctggccgccctgaggccccagtggtgcagcagggagatccccgaccccgccaacagcacctgcgccaagaagtaccccatcgccgaggagaagacccagctgcccctggacaggctgctgatcgactggcccacccccgaggaccccgagcccctggtgatcagcgaggtgctgcaccaggtgacccccgtgttcaggcaccccccctgcagcaactggccccagagggagaagggcatccagggccaccaggccagcgagaaggacatgatgcacagcgccagcagcccccccccccccagggccctgcaggccgagagcaggcagctggtggacctgtacaaggtgctggagagcaggggcagcgaccccaagcccgagaaccccgcctgcccctggaccgtgctgcccgccggcgacctgcccacccacgacggctacctgcccagcaacatcgacgacctgcccagccacgaggcccccctggccgacagcctggaggagctggagccccagcacatcagcctgagcgtgttccccagcagcagcctgcaccccctgaccttcagctgcggcgacaagctgaccctggaccagctgaagatgaggtgcgacagcctgatgctg

SEQ ID NO: 102 - protein, 372 aa, synthetic construct
MDPDLEIRAAFLRQRNTALRTEVAELEQEVQRLENEVSQYETRYGPLGGGKGGGGSGGGGSGGGGSMDPDLEIRAAFLRQRNTALRTEVAELEQEVQRLENEVSQYETRYGPLGGGKSHGNEREFCLQGKANWMAFVAPSICIAIIMVGIFSTHYFQQKVFVLLAALRPQWCSREIPDPANSTCAKKYPIAEEKTQLPLDRLLIDWPTPEDPEPLVISEVLHQVTPVFRHPPCSNWPQREKGIQGHQASEKDMMHSASSPPPPRALQAESRQLVDLYKVLESRGSDPKPENPACPWTVLPAGDLPTHDGYLPSNIDDLPSHEAPLADSLEELEPQHISLSVFPSSSLHPLTFSCGDKLTLDQLKMRCDSLML

SEQ ID NO: 103 - DNA, 1116 bp, synthetic construct
  source 1..1116: mol_type = other DNA
atggaccccgacctggaaatcagagccgccttcctgcggcagagaaacacagccctgagaacagaggtggccgagctggaacaagaggtgcagcggctggaaaacgaggtgtcccagtacgagacaagatacggccctctcggcggaggaaaaggtggcggaggttctggcggaggcggaagtggtggtggcggatctatggatcctgacctcgagattagggccgcctttctgagacagcggaacaccgctctgaggaccgaagtggccgaactcgagcaagaagtccagagactggaaaatgaagtctctcagtacgaaacccgctacggacctcttggaggcggcaaaagccacggcaacgagagggagttctgcctgcagggcaaggccaactggatggccttcgtggcccccagcatctgcatcgccatcatcatggtgggcatcttcagcacccactacttccagcagaaggtgttcgtgctgctggccgccctgaggccccagtggtgcagcagggagatccccgaccccgccaacagcacctgcgccaagaagtaccccatcgccgaggagaagacccagctgcccctggacaggctgctgatcgactggcccacccccgaggaccccgagcccctggtgatcagcgaggtgctgcaccaggtgacccccgtgttcaggcaccccccctgcagcaactggccccagagggagaagggcatccagggccaccaggccagcgagaaggacatgatgcacagcgccagcagcccccccccccccagggccctgcaggccgagagcaggcagctggtggacctgtacaaggtgctggagagcaggggcagcgaccccaagcccgagaaccccgcctgcccctggaccgtgctgcccgccggcgacctgcccacccacgacggctacctgcccagcaacatcgacgacctgcccagccacgaggcccccctggccgacagcctggaggagctggagccccagcacatcagcctgagcgtgttccccagcagcagcctgcaccccctgaccttcagctgcggcgacaagctgaccctggaccagctgaagatgaggtgcgacagcctgatgctg

SEQ ID NO: 104 - protein, 15 aa, synthetic construct
ISTGHLTSDNRGDIG

SEQ ID NO: 105 - DNA, 45 bp, synthetic construct
  source 1..45: mol_type = other DNA
atcagcaccggccacctgaccagcgacaacaggggcgacatcggc

SEQ ID NO: 106 - protein, 21 aa, synthetic construct
LLLGMIVFAVMLSILSLIGIF

SEQ ID NO: 107 - DNA, 63 bp, synthetic construct
  source 1..63: mol_type = other DNA
ctgctgctgggcatgatcgtgttcgccgtgatgctgagcatcctgagcctgatcggcatcttc

SEQ ID NO: 108 - protein, 253 aa, synthetic construct
NRSFRTGIKRRILLLIPKWLYEDIPNMKNSNVVKMLQENSELMNNNSSEQVLYVDPMITEIKEIFIPEHKPTDYKKENTGPLETRDYPQNSLFDNTTVVYIPDLNTGYKPQISNFLPEGSHLSNNNEITSLTLKPPVDSLDSGNNPRLQKHPNFAFSVSSVNSLSNTIFLGELSLILNQGECSSPDIQNSVEEETTMLLENDSPSETIPEQTLLPDEFVSCLGIVNEELPSINTYFPQNILESHFNRISLLEK

SEQ ID NO: 109 - DNA, 759 bp, synthetic construct
  source 1..759: mol_type = other DNA
aacaggagcttcaggaccggcatcaagaggaggatcctgctgctgatccccaagtggctgtacgaggacatccccaacatgaagaacagcaacgtggtgaagatgctgcaggagaacagcgagctgatgaacaacaacagcagcgagcaggtgctgtacgtggaccccatgatcaccgagatcaaggagatcttcatccccgagcacaagcccaccgactacaagaaggagaacaccggccccctggagaccagggactacccccagaacagcctgttcgacaacaccaccgtggtgtacatccccgacctgaacaccggctacaagccccagatcagcaacttcctgcccgagggcagccacctgagcaacaacaacgagatcaccagcctgaccctgaagccccccgtggacagcctggacagcggcaacaaccccaggctgcagaagcaccccaacttcgccttcagcgtgagcagcgtgaacagcctgagcaacaccatcttcctgggcgagctgagcctgatcctgaaccagggcgagtgcagcagccccgacatccagaacagcgtggaggaggagaccaccatgctgctggagaacgacagccccagcgagaccatccccgagcagaccctgctgcccgacgagttcgtgagctgcctgggcatcgtgaacgaggagctgcccagcatcaacacctacttcccccagaacatcctggagagccacttcaacaggatcagcctgctggagaag

SEQ ID NO: 110 - protein, 289 aa, synthetic construct
ISTGHLTSDNRGDIGLLLGMIVFAVMLSILSLIGIFNRSFRTGIKRRILLLIPKWLYEDIPNMKNSNVVKMLQENSELMNNNSSEQVLYVDPMITEIKEIFIPEHKPTDYKKENTGPLETRDYPQNSLFDNTTVVYIPDLNTGYKPQISNFLPEGSHLSNNNEITSLTLKPPVDSLDSGNNPRLQKHPNFAFSVSSVNSLSNTIFLGELSLILNQGECSSPDIQNSVEEETTMLLENDSPSETIPEQTLLPDEFVSCLGIVNEELPSINTYFPQNILESHFNRISLLEK

SEQ ID NO: 111 - DNA, 867 bp, synthetic construct
  source 1..867: mol_type = other DNA
atcagcaccggccacctgaccagcgacaacaggggcgacatcggcctgctgctgggcatgatcgtgttcgccgtgatgctgagcatcctgagcctgatcggcatcttcaacaggagcttcaggaccggcatcaagaggaggatcctgctgctgatccccaagtggctgtacgaggacatccccaacatgaagaacagcaacgtggtgaagatgctgcaggagaacagcgagctgatgaacaacaacagcagcgagcaggtgctgtacgtggaccccatgatcaccgagatcaaggagatcttcatccccgagcacaagcccaccgactacaagaaggagaacaccggccccctggagaccagggactacccccagaacagcctgttcgacaacaccaccgtggtgtacatccccgacctgaacaccggctacaagccccagatcagcaacttcctgcccgagggcagccacctgagcaacaacaacgagatcaccagcctgaccctgaagccccccgtggacagcctggacagcggcaacaaccccaggctgcagaagcaccccaacttcgccttcagcgtgagcagcgtgaacagcctgagcaacaccatcttcctgggcgagctgagcctgatcctgaaccagggcgagtgcagcagccccgacatccagaacagcgtggaggaggagaccaccatgctgctggagaacgacagccccagcgagaccatccccgagcagaccctgctgcccgacgagttcgtgagctgcctgggcatcgtgaacgaggagctgcccagcatcaacacctacttcccccagaacatcctggagagccacttcaacaggatcagcctgctggagaag

SEQ ID NO: 112 - protein, 406 aa, synthetic construct
MDPDLEIRAAFLRQRNTALRTEVAELEQEVQRLENEVSQYETRYGPLGGGKGGGGSGGGGSGGGGSMDPDLEIRAAFLRQRNTALRTEVAELEQEVQRLENEVSQYETRYGPLGGGKISTGHLTSDNRGDIGLLLGMIVFAVMLSILSLIGIFNRSFRTGIKRRILLLIPKWLYEDIPNMKNSNVVKMLQENSELMNNNSSEQVLYVDPMITEIKEIFIPEHKPTDYKKENTGPLETRDYPQNSLFDNTTVVYIPDLNTGYKPQISNFLPEGSHLSNNNEITSLTLKPPVDSLDSGNNPRLQKHPNFAFSVSSVNSLSNTIFLGELSLILNQGECSSPDIQNSVEEETTMLLENDSPSETIPEQTLLPDEFVSCLGIVNEELPSINTYFPQNILESHFNRISLLEK

SEQ ID NO: 113 - DNA, 1218 bp, synthetic construct
  source 1..1218: mol_type = other DNA
atggaccccgacctggaaatcagagccgccttcctgcggcagagaaacacagccctgagaacagaggtggccgagctggaacaagaggtgcagcggctggaaaacgaggtgtcccagtacgagacaagatacggccctctcggcggaggaaaaggtggcggaggttctggcggaggcggaagtggtggtggcggatctatggatcctgacctcgagattagggccgcctttctgagacagcggaacaccgctctgaggaccgaagtggccgaactcgagcaagaagtccagagactggaaaatgaagtctctcagtacgaaacccgctacggacctcttggaggcggcaaaatcagcaccggccacctgaccagcgacaacaggggcgacatcggcctgctgctgggcatgatcgtgttcgccgtgatgctgagcatcctgagcctgatcggcatcttcaacaggagcttcaggaccggcatcaagaggaggatcctgctgctgatccccaagtggctgtacgaggacatccccaacatgaagaacagcaacgtggtgaagatgctgcaggagaacagcgagctgatgaacaacaacagcagcgagcaggtgctgtacgtggaccccatgatcaccgagatcaaggagatcttcatccccgagcacaagcccaccgactacaagaaggagaacaccggccccctggagaccagggactacccccagaacagcctgttcgacaacaccaccgtggtgtacatccccgacctgaacaccggctacaagccccagatcagcaacttcctgcccgagggcagccacctgagcaacaacaacgagatcaccagcctgaccctgaagccccccgtggacagcctggacagcggcaacaaccccaggctgcagaagcaccccaacttcgccttcagcgtgagcagcgtgaacagcctgagcaacaccatcttcctgggcgagctgagcctgatcctgaaccagggcgagtgcagcagccccgacatccagaacagcgtggaggaggagaccaccatgctgctggagaacgacagccccagcgagaccatccccgagcagaccctgctgcccgacgagttcgtgagctgcctgggcatcgtgaacgaggagctgcccagcatcaacacctacttcccccagaacatcctggagagccacttcaacaggatcagcctgctggagaag

SEQ ID NO: 114 - protein, 15 aa, synthetic construct
EECISLTRQYFTVTN

SEQ ID NO: 115 - DNA, 45 bp, synthetic construct
  source 1..45: mol_type = other DNA
gaggagtgcatcagcctgaccaggcagtacttcaccgtgaccaac

SEQ ID NO: 116 - protein, 21 aa, synthetic construct
VIIFFAFVLLLSGALAYCLAL

SEQ ID NO: 117 - DNA, 63 bp, synthetic construct
  source 1..63: mol_type = other DNA
gtgatcatcttcttcgccttcgtgctgctgctgagcggcgccctggcctactgcctggccctg

SEQ ID NO: 118 - protein, 322 aa, synthetic construct
QLYVRRRKKLPSVLLFKKPSPFIFISQRPSPETQDTIHPLDEEAFLKVSPELKNLDLHGSTDSGFGSTKPSLQTEEPQFLLPDPHPQADRTLGNREPPVLGDSCSSGSSNSTDSGICLQEPSLSPSTGPTWEQQVGSNSRGQDDSGIDLVQNSEGRAGDTQGGSALGHHSPPEPEVPGEEDPAAVAFQGYLRQTRCAEEKATKTGCLEEESPLTDGLGPKFGRCLVDEAGLHPPALAKGYLKQDPLEMTLASSGAPTGQWNQPTEEWSLLALSSCSDLGISDWSFAHDLAPLGCVAAPGGLLGSFNSDLVTLPLISSLQSSE

SEQ ID NO: 119 - DNA, 966 bp, synthetic construct
  source 1..966: mol_type = other DNA
cagctgtacgtgaggaggaggaagaagctgcccagcgtgctgctgttcaagaagcccagccccttcatcttcatcagccagaggcccagccccgagacccaggacaccatccaccccctggacgaggaggccttcctgaaggtgagccccgagctgaagaacctggacctgcacggcagcaccgacagcggcttcggcagcaccaagcccagcctgcagaccgaggagccccagttcctgctgcccgacccccacccccaggccgacaggaccctgggcaacagggagccccccgtgctgggcgacagctgcagcagcggcagcagcaacagcaccgacagcggcatctgcctgcaggagcccagcctgagccccagcaccggccccacctgggagcagcaggtgggcagcaacagcaggggccaggacgacagcggcatcgacctggtgcagaacagcgagggcagggccggcgacacccagggcggcagcgccctgggccaccacagcccccccgagcccgaggtgcccggcgaggaggaccccgccgccgtggccttccagggctacctgaggcagaccaggtgcgccgaggagaaggccaccaagaccggctgcctggaggaggagagccccctgaccgacggcctgggccccaagttcggcaggtgcctggtggacgaggccggcctgcacccccccgccctggccaagggctacctgaagcaggaccccctggagatgaccctggccagcagcggcgcccccaccggccagtggaaccagcccaccgaggagtggagcctgctggccctgagcagctgcagcgacctgggcatcagcgactggagcttcgcccacgacctggcccccctgggctgcgtggccgcccccggcggcctgctgggcagcttcaacagcgacctggtgaccctgcccctgatcagcagcctgcagagcagcgag

SEQ ID NO: 120 - protein, 358 aa, synthetic construct
EECISLTRQYFTVTNVIIFFAFVLLLSGALAYCLALQLYVRRRKKLPSVLLFKKPSPFIFISQRPSPETQDTIHPLDEEAFLKVSPELKNLDLHGSTDSGFGSTKPSLQTEEPQFLLPDPHPQADRTLGNREPPVLGDSCSSGSSNSTDSGICLQEPSLSPSTGPTWEQQVGSNSRGQDDSGIDLVQNSEGRAGDTQGGSALGHHSPPEPEVPGEEDPAAVAFQGYLRQTRCAEEKATKTGCLEEESPLTDGLGPKFGRCLVDEAGLHPPALAKGYLKQDPLEMTLASSGAPTGQWNQPTEEWSLLALSSCSDLGISDWSFAHDLAPLGCVAAPGGLLGSFNSDLVTLPLISSLQSSE

SEQ ID NO: 121 - DNA, 1074 bp, synthetic construct
  source 1..1074: mol_type = other DNA
gaggagtgcatcagcctgaccaggcagtacttcaccgtgaccaacgtgatcatcttcttcgccttcgtgctgctgctgagcggcgccctggcctactgcctggccctgcagctgtacgtgaggaggaggaagaagctgcccagcgtgctgctgttcaagaagcccagccccttcatcttcatcagccagaggcccagccccgagacccaggacaccatccaccccctggacgaggaggccttcctgaaggtgagccccgagctgaagaacctggacctgcacggcagcaccgacagcggcttcggcagcaccaagcccagcctgcagaccgaggagccccagttcctgctgcccgacccccacccccaggccgacaggaccctgggcaacagggagccccccgtgctgggcgacagctgcagcagcggcagcagcaacagcaccgacagcggcatctgcctgcaggagcccagcctgagccccagcaccggccccacctgggagcagcaggtgggcagcaacagcaggggccaggacgacagcggcatcgacctggtgcagaacagcgagggcagggccggcgacacccagggcggcagcgccctgggccaccacagcccccccgagcccgaggtgcccggcgaggaggaccccgccgccgtggccttccagggctacctgaggcagaccaggtgcgccgaggagaaggccaccaagaccggctgcctggaggaggagagccccctgaccgacggcctgggccccaagttcggcaggtgcctggtggacgaggccggcctgcacccccccgccctggccaagggctacctgaagcaggaccccctggagatgaccctggccagcagcggcgcccccaccggccagtggaaccagcccaccgaggagtggagcctgctggccctgagcagctgcagcgacctgggcatcagcgactggagcttcgcccacgacctggcccccctgggctgcgtggccgcccccggcggcctgctgggcagcttcaacagcgacctggtgaccctgcccctgatcagcagcctgcagagcagcgag

SEQ ID NO: 122 - protein, 475 aa, synthetic construct
MDPDLEIEAAFLERENTALETRVAELRQRVQRLRNRVSQYRTRYGPLGGGKGGGGSGGGGSGGGGSMDPDLEIEAAFLERENTALETRVAELRQRVQRLRNRVSQYRTRYGPLGGGKEECISLTRQYFTVTNVIIFFAFVLLLSGALAYCLALQLYVRRRKKLPSVLLFKKPSPFIFISQRPSPETQDTIHPLDEEAFLKVSPELKNLDLHGSTDSGFGSTKPSLQTEEPQFLLPDPHPQADRTLGNREPPVLGDSCSSGSSNSTDSGICLQEPSLSPSTGPTWEQQVGSNSRGQDDSGIDLVQNSEGRAGDTQGGSALGHHSPPEPEVPGEEDPAAVAFQGYLRQTRCAEEKATKTGCLEEESPLTDGLGPKFGRCLVDEAGLHPPALAKGYLKQDPLEMTLASSGAPTGQWNQPTEEWSLLALSSCSDLGISDWSFAHDLAPLGCVAAPGGLLGSFNSDLVTLPLISSLQSSE

SEQ ID NO: 123 - DNA, 1425 bp, synthetic construct
  source 1..1425: mol_type = other DNA
atggaccccgacctggaaatcgaggccgccttcctggaaagagagaacacagccctggaaaccagagtggccgagctgagacagagagtgcagcggctgagaaaccgggtgtcccagtacagaaccagatacggacctctcggcggaggaaaaggtggcggaggttctggcggaggcggctctggtggtggtggatctatggatcctgacctcgagattgaagccgcctttctcgagcgcgagaataccgctctcgagacaagagtggctgaactgcggcagagggtccagagactgaggaacagagtgtctcagtaccggacacgctacggccctcttggaggtggaaaagaggagtgcatcagcctgaccaggcagtacttcaccgtgaccaacgtgatcatcttcttcgccttcgtgctgctgctgagcggcgccctggcctactgcctggccctgcagctgtacgtgaggaggaggaagaagctgcccagcgtgctgctgttcaagaagcccagccccttcatcttcatcagccagaggcccagccccgagacccaggacaccatccaccccctggacgaggaggccttcctgaaggtgagccccgagctgaagaacctggacctgcacggcagcaccgacagcggcttcggcagcaccaagcccagcctgcagaccgaggagccccagttcctgctgcccgacccccacccccaggccgacaggaccctgggcaacagggagccccccgtgctgggcgacagctgcagcagcggcagcagcaacagcaccgacagcggcatctgcctgcaggagcccagcctgagccccagcaccggccccacctgggagcagcaggtgggcagcaacagcaggggccaggacgacagcggcatcgacctggtgcagaacagcgagggcagggccggcgacacccagggcggcagcgccctgggccaccacagcccccccgagcccgaggtgcccggcgaggaggaccccgccgccgtggccttccagggctacctgaggcagaccaggtgcgccgaggagaaggccaccaagaccggctgcctggaggaggagagccccctgaccgacggcctgggccccaagttcggcaggtgcctggtggacgaggccggcctgcacccccccgccctggccaagggctacctgaagcaggaccccctggagatgaccctggccagcagcggcgcccccaccggccagtggaaccagcccaccgaggagtggagcctgctggccctgagcagctgcagcgacctgggcatcagcgactggagcttcgcccacgacctggcccccctgggctgcgtggccgcccccggcggcctgctgggcagcttcaacagcgacctggtgaccctgcccctgatcagcagcctgcagagcagcgag

SEQ ID NO: 124 - protein, 15 aa, synthetic construct
EPVCEQTTHDETVPS

SEQ ID NO: 125 - DNA, 45 bp, synthetic construct
  source 1..45: mol_type = other DNA
gagcccgtgtgcgagcagaccacccacgacgagaccgtgcccagc

SEQ ID NO: 126 - protein, 22 aa, synthetic construct
WMVAVILMASVFMVCLALLGCF

SEQ ID NO: 127 - DNA, 66 bp, synthetic construct
  source 1..66: mol_type = other DNA
tggatggtggccgtgatcctgatggccagcgtgttcatggtgtgcctggccctgctgggctgcttc

SEQ ID NO: 128 - protein, 83 aa, synthetic construct
ALLWCVYKKTKYAFSPRNSLPQHLKEFLGHPHHNTLLFFSFPLSDENDVFDKLSVIAEDSESGKQNPGDSCSLGTPPGQGPQS

SEQ ID NO: 129 - DNA, 249 bp, synthetic construct
  source 1..249: mol_type = other DNA
gccctgctgtggtgcgtgtacaagaagaccaagtacgccttcagccccaggaacagcctgccccagcacctgaaggagttcctgggccacccccaccacaacaccctgctgttcttcagcttccccctgagcgacgagaacgacgtgttcgacaagctgagcgtgatcgccgaggacagcgagagcggcaagcagaaccccggcgacagctgcagcctgggcaccccccccggccagggcccccagagc

SEQ ID NO: 130 - protein, 120 aa, synthetic construct
EPVCEQTTHDETVPSWMVAVILMASVFMVCLALLGCFALLWCVYKKTKYAFSPRNSLPQHLKEFLGHPHHNTLLFFSFPLSDENDVFDKLSVIAEDSESGKQNPGDSCSLGTPPGQGPQS

SEQ ID NO: 131 - DNA, 360 bp, synthetic construct
  source 1..360: mol_type = other DNA
gagcccgtgtgcgagcagaccacccacgacgagaccgtgcccagctggatggtggccgtgatcctgatggccagcgtgttcatggtgtgcctggccctgctgggctgcttcgccctgctgtggtgcgtgtacaagaagaccaagtacgccttcagccccaggaacagcctgccccagcacctgaaggagttcctgggccacccccaccacaacaccctgctgttcttcagcttccccctgagcgacgagaacgacgtgttcgacaagctgagcgtgatcgccgaggacagcgagagcggcaagcagaaccccggcgacagctgcagcctgggcaccccccccggccagggcccccagagc

SEQ ID NO: 132 - protein, 237 aa, synthetic construct
MDPDLEIRAAFLRQRNTALRTEVAELEQEVQRLENEVSQYETRYGPLGGGKGGGGSGGGGSGGGGSMDPDLEIRAAFLRQRNTALRTEVAELEQEVQRLENEVSQYETRYGPLGGGKEPVCEQTTHDETVPSWMVAVILMASVFMVCLALLGCFALLWCVYKKTKYAFSPRNSLPQHLKEFLGHPHHNTLLFFSFPLSDENDVFDKLSVIAEDSESGKQNPGDSCSLGTPPGQGPQS

SEQ ID NO: 133 - DNA, 711 bp, synthetic construct
  source 1..711: mol_type = other DNA
atggaccccgacctggaaatcagagccgccttcctgcggcagagaaacacagccctgagaacagaggtggccgagctggaacaagaggtgcagcggctggaaaacgaggtgtcccagtacgagacaagatacggccctctcggcggaggaaaaggtggcggaggttctggcggaggcggaagtggtggtggcggatctatggatcctgacctcgagattagggccgcctttctgagacagcggaacaccgctctgaggaccgaagtggccgaactcgagcaagaagtccagagactggaaaatgaagtctctcagtacgaaacccgctacggacctcttggaggcggcaaagagcccgtgtgcgagcagaccacccacgacgagaccgtgcccagctggatggtggccgtgatcctgatggccagcgtgttcatggtgtgcctggccctgctgggctgcttcgccctgctgtggtgcgtgtacaagaagaccaagtacgccttcagccccaggaacagcctgccccagcacctgaaggagttcctgggccacccccaccacaacaccctgctgttcttcagcttccccctgagcgacgagaacgacgtgttcgacaagctgagcgtgatcgccgaggacagcgagagcggcaagcagaaccccggcgacagctgcagcctgggcaccccccccggccagggcccccagagc

SEQ ID NO: 134 - protein, 15 aa, synthetic construct
EFTFTTPKFAQGEIE

SEQ ID NO: 135 - DNA, 45 bp, synthetic construct
  source 1..45: mol_type = other DNA
gagttcaccttcaccacccccaagttcgcccagggcgagatcgag

SEQ ID NO: 136 - protein, 22 aa, synthetic construct
AIVVPVCLAFLLTTLLGVLFCF

SEQ ID NO: 137 - DNA, 66 bp, synthetic construct
  source 1..66: mol_type = other DNA
gccatcgtggtgcccgtgtgcctggccttcctgctgaccaccctgctgggcgtgctgttctgcttc

SEQ ID NO: 138 - protein, 277 aa, synthetic construct
NKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSISSSDENESSQNTSSTVQYSTVVHSGYRHQVPSVQVFSRSESTQPLLDSEERPEDLQLVDHVDGGDGILPRQQYFKQNCSQHESSPDISHFERSKQVSSVNEEDFVRLKQQISDHISQSCGSGQMKMFQEVSAADAFGPGTEGQVERFETVGMEAATDEGMPKSYLPQTVRQGGYMPQ

SEQ ID NO: 139 - DNA, 831 bp, synthetic construct
  source 1..831: mol_type = other DNA
aacaagagggacctgatcaagaagcacatctggcccaacgtgcccgaccccagcaagagccacatcgcccagtggagcccccacaccccccccaggcacaacttcaacagcaaggaccagatgtacagcgacggcaacttcaccgacgtgagcgtggtggagatcgaggccaacgacaagaagcccttccccgaggacctgaagagcctggacctgttcaagaaggagaagatcaacaccgagggccacagcagcggcatcggcggcagcagctgcatgagcagcagcaggcccagcatcagcagcagcgacgagaacgagagcagccagaacaccagcagcaccgtgcagtacagcaccgtggtgcacagcggctacaggcaccaggtgcccagcgtgcaggtgttcagcaggagcgagagcacccagcccctgctggacagcgaggagaggcccgaggacctgcagctggtggaccacgtggacggcggcgacggcatcctgcccaggcagcagtacttcaagcagaactgcagccagcacgagagcagccccgacatcagccacttcgagaggagcaagcaggtgagcagcgtgaacgaggaggacttcgtgaggctgaagcagcagatcagcgaccacatcagccagagctgcggcagcggccagatgaagatgttccaggaggtgagcgccgccgacgccttcggccccggcaccgagggccaggtggagaggttcgagaccgtgggcatggaggccgccaccgacgagggcatgcccaagagctacctgccccagaccgtgaggcagggcggctacatgccccag

SEQ ID NO: 140 - protein, 314 aa, synthetic construct
EFTFTTPKFAQGEIEAIVVPVCLAFLLTTLLGVLFCFNKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSISSSDENESSQNTSSTVQYSTVVHSGYRHQVPSVQVFSRSESTQPLLDSEERPEDLQLVDHVDGGDGILPRQQYFKQNCSQHESSPDISHFERSKQVSSVNEEDFVRLKQQISDHISQSCGSGQMKMFQEVSAADAFGPGTEGQVERFETVGMEAATDEGMPKSYLPQTVRQGGYMPQ

SEQ ID NO: 141 - DNA, 942 bp, synthetic construct
  source 1..942: mol_type = other DNA
gagttcaccttcaccacccccaagttcgcccagggcgagatcgaggccatcgtggtgcccgtgtgcctggccttcctgctgaccaccctgctgggcgtgctgttctgcttcaacaagagggacctgatcaagaagcacatctggcccaacgtgcccgaccccagcaagagccacatcgcccagtggagcccccacaccccccccaggcacaacttcaacagcaaggaccagatgtacagcgacggcaacttcaccgacgtgagcgtggtggagatcgaggccaacgacaagaagcccttccccgaggacctgaagagcctggacctgttcaagaaggagaagatcaacaccgagggccacagcagcggcatcggcggcagcagctgcatgagcagcagcaggcccagcatcagcagcagcgacgagaacgagagcagccagaacaccagcagcaccgtgcagtacagcaccgtggtgcacagcggctacaggcaccaggtgcccagcgtgcaggtgttcagcaggagcgagagcacccagcccctgctggacagcgaggagaggcccgaggacctgcagctggtggaccacgtggacggcggcgacggcatcctgcccaggcagcagtacttcaagcagaactgcagccagcacgagagcagccccgacatcagccacttcgagaggagcaagcaggtgagcagcgtgaacgaggaggacttcgtgaggctgaagcagcagatcagcgaccacatcagccagagctgcggcagcggccagatgaagatgttccaggaggtgagcgccgccgacgccttcggccccggcaccgagggccaggtggagaggttcgagaccgtgggcatggaggccgccaccgacgagggcatgcccaagagctacctgccccagaccgtgaggcagggcggctacatgccccag

SEQ ID NO: 142 - protein, 431 aa, synthetic construct
MDPDLEIEAAFLERENTALETRVAELRQRVQRLRNRVSQYRTRYGPLGGGKGGGGSGGGGSGGGGSMDPDLEIEAAFLERENTALETRVAELRQRVQRLRNRVSQYRTRYGPLGGGKEFTFTTPKFAQGEIEAIVVPVCLAFLLTTLLGVLFCFNKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSISSSDENESSQNTSSTVQYSTVVHSGYRHQVPSVQVFSRSESTQPLLDSEERPEDLQLVDHVDGGDGILPRQQYFKQNCSQHESSPDISHFERSKQVSSVNEEDFVRLKQQISDHISQSCGSGQMKMFQEVSAADAFGPGTEGQVERFETVGMEAATDEGMPKSYLPQTVRQGGYMPQ

SEQ ID NO: 143 - DNA, 1293 bp, synthetic construct
  source 1..1293: mol_type = other DNA
atggaccccgacctggaaatcgaggccgccttcctggaaagagagaacacagccctggaaaccagagtggccgagctgagacagagagtgcagcggctgagaaaccgggtgtcccagtacagaaccagatacggacctctcggcggaggaaaaggtggcggaggttctggcggaggcggctctggtggtggtggatctatggatcctgacctcgagattgaagccgcctttctcgagcgcgagaataccgctctcgagacaagagtggctgaactgcggcagagggtccagagactgaggaacagagtgtctcagtaccggacacgctacggccctcttggaggtggaaaagagttcaccttcaccacccccaagttcgcccagggcgagatcgaggccatcgtggtgcccgtgtgcctggccttcctgctgaccaccctgctgggcgtgctgttctgcttcaacaagagggacctgatcaagaagcacatctggcccaacgtgcccgaccccagcaagagccacatcgcccagtggagcccccacaccccccccaggcacaacttcaacagcaaggaccagatgtacagcgacggcaacttcaccgacgtgagcgtggtggagatcgaggccaacgacaagaagcccttccccgaggacctgaagagcctggacctgttcaagaaggagaagatcaacaccgagggccacagcagcggcatcggcggcagcagctgcatgagcagcagcaggcccagcatcagcagcagcgacgagaacgagagcagccagaacaccagcagcaccgtgcagtacagcaccgtggtgcacagcggctacaggcaccaggtgcccagcgtgcaggtgttcagcaggagcgagagcacccagcccctgctggacagcgaggagaggcccgaggacctgcagctggtggaccacgtggacggcggcgacggcatcctgcccaggcagcagtacttcaagcagaactgcagccagcacgagagcagccccgacatcagccacttcgagaggagcaagcaggtgagcagcgtgaacgaggaggacttcgtgaggctgaagcagcagatcagcgaccacatcagccagagctgcggcagcggccagatgaagatgttccaggaggtgagcgccgccgacgccttcggccccggcaccgagggccaggtggagaggttcgagaccgtgggcatggaggccgccaccgacgagggcatgcccaagagctacctgccccagaccgtgaggcagggcggctacatgccccag

SEQ ID NO: 144 - protein, 15 aa, synthetic construct
PILRLHLPDNTLRWK

SEQ ID NO: 145 - DNA, 45 bp, synthetic construct
  source 1..45: mol_type = other DNA
cccatcctgaggctgcacctgcccgacaacaccctgaggtggaag

SEQ ID NO: 146 - protein, 21 aa, synthetic construct
VLPGILFLWGLFLLGCGLSLA

SEQ ID NO: 147 - DNA, 63 bp, synthetic construct
  source 1..63: mol_type = other DNA
gtgctgcccggcatcctgttcctgtggggcctgttcctgctgggctgcggcctgagcctggcc

SEQ ID NO: 148 - protein, 122 aa, synthetic construct
TSGRCYHLRHKVLPRWVWEKVPDPANSSSGQPHMEQVPEAQPLGDLPILEVEEMEPPPVMESSQPAQATAPLDSGYEKHFLPTPEELGLLGPPRPQVLAESGKQNPGDSCSLGTPPGQGPQS

SEQ ID NO: 149 - DNA, 297 bp, synthetic construct
  source 1..297: mol_type = other DNA
accagcggcaggtgctaccacctgaggcacaaggtgctgcccaggtgggtgtgggagaaggtgcccgaccccgccaacagcagcagcggccagccccacatggagcaggtgcccgaggcccagcccctgggcgacctgcccatcctggaggtggaggagatggagcccccccccgtgatggagagcagccagcccgcccaggccaccgcccccctggacagcggctacgagaagcacttcctgcccacccccgaggagctgggcctgctgggcccccccaggccccaggtgctggcc

SEQ ID NO: 150 - protein, 158 aa, synthetic construct
PILRLHLPDNTLRWKVLPGILFLWGLFLLGCGLSLATSGRCYHLRHKVLPRWVWEKVPDPANSSSGQPHMEQVPEAQPLGDLPILEVEEMEPPPVMESSQPAQATAPLDSGYEKHFLPTPEELGLLGPPRPQVLAESGKQNPGDSCSLGTPPGQGPQS

SEQ ID NO: 151 - DNA, 405 bp, synthetic construct
  source 1..405: mol_type = other DNA
cccatcctgaggctgcacctgcccgacaacaccctgaggtggaaggtgctgcccggcatcctgttcctgtggggcctgttcctgctgggctgcggcctgagcctggccaccagcggcaggtgctaccacctgaggcacaaggtgctgcccaggtgggtgtgggagaaggtgcccgaccccgccaacagcagcagcggccagccccacatggagcaggtgcccgaggcccagcccctgggcgacctgcccatcctggaggtggaggagatggagcccccccccgtgatggagagcagccagcccgcccaggccaccgcccccctggacagcggctacgagaagcacttcctgcccacccccgaggagctgggcctgctgggcccccccaggccccaggtgctggcc

SEQ ID NO: 152 - protein, 275 aa, synthetic construct
MDPDLEIRAAFLRQRNTALRTEVAELEQEVQRLENEVSQYETRYGPLGGGKGGGGSGGGGSGGGGSMDPDLEIRAAFLRQRNTALRTEVAELEQEVQRLENEVSQYETRYGPLGGGKPILRLHLPDNTLRWKVLPGILFLWGLFLLGCGLSLATSGRCYHLRHKVLPRWVWEKVPDPANSSSGQPHMEQVPEAQPLGDLPILEVEEMEPPPVMESSQPAQATAPLDSGYEKHFLPTPEELGLLGPPRPQVLAESGKQNPGDSCSLGTPPGQGPQS

SEQ ID NO: 153 - DNA, 756 bp, synthetic construct
  source 1..756: mol_type = other DNA
atggaccccgacctggaaatcagagccgccttcctgcggcagagaaacacagccctgagaacagaggtggccgagctggaacaagaggtgcagcggctggaaaacgaggtgtcccagtacgagacaagatacggccctctcggcggaggaaaaggtggcggaggttctggcggaggcggaagtggtggtggcggatctatggatcctgacctcgagattagggccgcctttctgagacagcggaacaccgctctgaggaccgaagtggccgaactcgagcaagaagtccagagactggaaaatgaagtctctcagtacgaaacccgctacggacctcttggaggcggcaaacccatcctgaggctgcacctgcccgacaacaccctgaggtggaaggtgctgcccggcatcctgttcctgtggggcctgttcctgctgggctgcggcctgagcctggccaccagcggcaggtgctaccacctgaggcacaaggtgctgcccaggtgggtgtgggagaaggtgcccgaccccgccaacagcagcagcggccagccccacatggagcaggtgcccgaggcccagcccctgggcgacctgcccatcctggaggtggaggagatggagcccccccccgtgatggagagcagccagcccgcccaggccaccgcccccctggacagcggctacgagaagcacttcctgcccacccccgaggagctgggcctgctgggcccccccaggccccaggtgctggcc

SEQ ID NO: 154 - protein, 764 aa, synthetic construct
MDPDLEIEAAFLERENTALETRVAELRQRVQRLRNRVSQYRTRYGPLGGGKGGGGSGGGGSGGGGSMDPDLEIEAAFLERENTALETRVAELRQRVQRLRNRVSQYRTRYGPLGGGKPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLVSDPVIFQTQSEELKEGWNPHLLLLLLLVIVFIPAFWSLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 155 - DNA, 2293 bp, synthetic construct
  source 1..2293: mol_type = other DNA
atggaccccgacctggaaatcgaggccgccttcctggaaagagagaacacagccctggaaaccagagtggccgagctgagacagagagtgcagcggctgagaaaccgggtgtcccagtacagaaccagatacggacctctcggcggaggaaaaggtggcggaggttctggcggaggcggctctggtggtggtggatctatggatcctgacctcgagattgaagccgcctttctcgagcgcgagaataccgctctcgagacaagagtggctgaactgcggcagagggtccagagactgaggaacagagtgtctcagtaccggacacgctacggccctcttggaggtggaaaacctctggccttcagaacaaaacctgccgctctgggcaaagacacaatcccctggctgggacatctgctcgttggactgtctggcgccttcggcttcatcatcctggtgtacctgctgatcaactgccggaacacaggcccttggctgaagaaagtgctgaagtgcaacacccctgatccgagcaagttctttagccagctgagcagcgagcatggcggcgacgttcagaaatggctgtctagcccatttcctagcagcagcttcagccctggtggactggcccctgagattagccctctggaagtgctggaacgggacaaagtgacccagctgctcctgcagcaggataaggtgccagaacctgccagcctgtccagcaatcacagcctgaccagctgctttaccaaccagggctacttcttcttccatctgcctgacgctctcgagatcgaggcctgccaggtgtacttcacctacgatccctacagcgaggaagatcccgacgaaggcgttgcaggcgctcctacaggatcttctccacagcctctgcagccactgtccggcgaggatgatgcctactgcacctttccaagccgggacgacctgctgctgtttagccctagtctgctcggcggaccttctccaccatctacagctccaggtggaagcggagccggcgaagaaagaatgcctcctagcctgcaagagcgggtgcccagagattgggatcctcaacctctcggacctcctacaccaggcgtgccagacctggtggattttcagcctcctccagagctggtgctgagagaagctggcgaagaagtgccagacgccggacctagagagggcgttagctttccttggagcagacctcctggacagggcgagttcagagccctgaatgctagactgcccctgaacaccgatgcctacctgtctctgcaagaactgcagggacaagaccccacacacctcgttgagcgaccccgtgatctttcagacccagagcgaggaactgaaagaaggctggaaccctcacctcctgctgttgctgctgctggtcatcgtgttcatccccgccttttggagcctgaaaacacaccctctgtggcggctgtggaagaaaatctgggccgtgccatctcctgagcggttcttcatgcctctgtacaagggctgcagcggcgacttcaagaaatgggtcggagccccttttaccggcagctctctggaacttggaccttggagccctgaggtgcccagcacactggaagtgtacagctgtcaccctcctagaagccccgccaagagactgcagctgacagagctgcaagagcctgccgagctggtggaatctgatggcgtgcccaagcctagcttctggcccacagctcagaatagcggcggctctgcctacagcgaggaaagagatagaccctacggcctggtgtccatcgacaccgtgacagtgctggatgccgagggaccttgtacctggccttgtagctgcgaggacgatggctaccctgctctggatctggacgctggccttgagccttctccaggactggaagatcctctgctggacgccggaacaaccgtgctgtcttgtggctgtgtgtctgccggatctcctggacttggaggccctctgggaagcctgctggatagactgaaacctcctctggccgacggcgaagattgggctggtggacttccttggggcggaagatctccaggcggagtgtctgagtctgaagccggttctccactggccggcctggacatggataccttcgattctggcttcgtgggcagcgactgtagcagccctgtggaatgcgacttcacaagccctggcgacgagggcccacctagaagctatctgagacagtgggtcgtgatccctccacctctgtctagtcctggacctcaggcttct

SEQ ID NO: 156 - protein, 239 aa, synthetic construct; identical to SEQ ID NO: 42

SEQ ID NO: 157 - DNA, 717 bp, synthetic construct; identical to SEQ ID NO: 43
  source 1..717: mol_type = other DNA

SEQ ID NO: 158 - protein, 117 aa, synthetic construct
MDPDLEIRAAFLRQRNTALRTEVAELEQEVQRLENEVSQYETRYGPLGGGKGGGGSGGGGSGGGGSMDPDLEIRAAFLRQRNTALRTEVAELEQEVQRLENEVSQYETRYGPLGGGK

SEQ ID NO: 159 - DNA, 351 bp, synthetic construct
  source 1..351: mol_type = other DNA
atggaccccgacctggaaatcagagccgccttcctgcggcagagaaacacagccctgagaacagaggtggccgagctggaacaagaggtgcagcggctggaaaacgaggtgtcccagtacgagacaagatacggccctctcggcggaggaaaaggtggcggaggttctggcggaggcggaagtggtggtggcggatctatggatcctgacctcgagattagggccgcctttctgagacagcggaacaccgctctgaggaccgaagtggccgaactcgagcaagaagtccagagactggaaaatgaagtctctcagtacgaaacccgctacggacctcttggaggcggcaaa

SEQ ID NO: 160 - protein, 117 aa, synthetic construct
MDPDLEIEAAFLERENTALETRVAELRQRVQRLRNRVSQYRTRYGPLGGGKGGGGSGGGGSGGGGSMDPDLEIEAAFLERENTALETRVAELRQRVQRLRNRVSQYRTRYGPLGGGK

SEQ ID NO: 161 - DNA, 351 bp, synthetic construct
  source 1..351: mol_type = other DNA
atggaccccgacctggaaatcgaggccgccttcctggaaagagagaacacagccctggaaaccagagtggccgagctgagacagagagtgcagcggctgagaaaccgggtgtcccagtacagaaccagatacggacctctcggcggaggaaaaggtggcggaggttctggcggaggcggctctggtggtggtggatctatggatcctgacctcgagattgaagccgcctttctcgagcgcgagaataccgctctcgagacaagagtggctgaactgcggcagagggtccagagactgaggaacagagtgtctcagtaccggacacgctacggccctcttggaggtggaaaa

SEQ ID NO: 162 - protein, 98 aa, synthetic construct
MSYYHHHHHHLESTSLYKKAGSEFDLERNRAAASRCRQKRKVWVQSLEKKAEDLSSLNGQLQSEVTLLRNEVAQLKQLLLAHKDCPVTAMQKKSGFLE

SEQ ID NO: 163 - protein, 80 aa, synthetic construct
MSYYHHHHHHLESTSLYKKAGSEFKRKLWVSSLEKKAEELTSQNIQLSNEVTLLRNEVAQLKQLLLAHKDCPVTALQKLE

SEQ ID NO: 164 - protein, 82 aa, synthetic construct
MSYYHHHHHHLESTSLYKKAGSEFQRKVWVMSLEKKAEELTQTNMQLQNEVSMLKNEVAQLKQLLLTHKDCPITAMQKESLE

SEQ ID NO: 165 - protein, 110 aa, synthetic construct
MSYYHHHHHHLESTSLYKKAGSGSCPEEDERKKRRRERNKIAAAKCRNKKKEKTECLQKESEKLESVNAELKAQIEELKNEKQHLIYMLNLHRPTCIVRAQNGRTPEDLE

SEQ ID NO: 166 - protein, 42 aa, synthetic construct
ERTEFLQRESERLELMNAELKTQIEELKQERQQLILMLNRHR

SEQ ID NO: 167 - protein, 88 aa, synthetic construct
MSYYHHHHHHLESTSLYKKAGSEFNKTAATRYRQKKRAEQEALTGECKELEKKNEALKERADSLAKEIQYLKDLIEEVRKARGKKRVP

SEQ ID NO: 168 - protein, 86 aa, synthetic construct
MSYYHHHHHHLESTSLYKKAGSEFKKGTRYRQKKRVEQEVLTGECKAVEKKNEALQERADSLAEEIQYMKDSIEEVCKARGKKRVL

SEQ ID NO: 169 - protein, 98 aa, synthetic construct
MSYYHHHHHHLESTSLYKKAGSEFNRKQKKRDQNKSAALRYRQRKRAEGEALEGECQGLEARNRELKERAESVEREIQYVKDLLIEVYKARSQRTRSC

SEQ ID NO: 170 - protein, 85 aa, synthetic construct
MSYYHHHHHHLESTSLYKKAGSEFACQSRKKKKEYMLGLEARLKAALSENEQLKKENGTLKRQLDEVVSENQRLKVPSPKRRVLE

SEQ ID NO: 171 - protein, 49 aa, synthetic construct
EYLQGLEARLQAVLADNQQLRRENAALRRRLEALLAENSELKLGSGNRK

SEQ ID NO: 172 - protein, 85 aa, synthetic construct
MSYYHHHHHHLESTSLYKKAGSEFCRLNRLKKKEYVMGLESRVRGLAAENQELRAENRELGKRVQALQEESRYLRAVLANETGLE

SEQ ID NO: 173 - protein, 68 aa, synthetic construct
MSYYHHHHHHLESTSLYKKAGSGSQKADTLHLESEDLEKQNAALRKEIKQLTEELKYFTSVLNSHELE

SEQ ID NO: 174 - protein, 89 aa, synthetic construct
MSYYHHHHHHLESTSLYKKAGSEFRSRKKQTQKADKLHEEYESLEQENTMLRREIGKLTEELKHLTEALKEHEKMCPLLLCPMNFVHLE

SEQ ID NO: 175 - protein, 42 aa, synthetic construct
DKADALHQQHESLEKDNLALRKEIQSLQAELAWWSRTLHVHE

SEQ ID NO: 176 - protein, 67 aa, synthetic construct
MSYYHHHHHHLESTSLYKKAGSGSQRNVETQQKVLELTSDNDRLRKRVEQLSRELDTLRGIFRQLLE

SEQ ID NO: 177 - protein, 99 aa, synthetic construct
MSYYHHHHHHLESTSLYKKAGSEFSDEYKIRRERNNIAVRKSRDKAKMRNLETQHKVLELTAENERLQKKVEQLSRELSTLRNLFKQLPEPLLASSGHC

SEQ ID NO: 178 - protein, 103 aa, synthetic construct
MSYYHHHHHHLESTSLYKKAGSEFGERNNMAVKKSRLKSKQKAQDTLQRVNQLKEENERLEAKIKLLTKELSVLKDLFLEHAHNLADNVQSISTENTTADGLE

SEQ ID NO: 179 - protein, 67 aa, synthetic construct
MSYYHHHHHHLESTSLYKKAGSGSRRNQEMQQKLVELSAENEKLHQRVEQLTRDLAGLRQFFKQLLE

SEQ ID NO: 180 - protein, 100 aa, synthetic construct
MSYYHHHHHHLESTSLYKKAGSEFHLEYRLRRERNNIAVRKSRDKAKRRILETQQKVLEYMAENERLRSRVEQLTQELDTLRNLFRQIPEAANLIKGVLE

SEQ ID NO: 181 - protein, 75 aa, synthetic construct
MSYYHHHHHHLESTSLYKKAGSEFRMKEKEQENERKVAQLAEENERLKQEIERLTREVEATRRALIDRMVNLHQA

SEQ ID NO: 182 - protein, 67 aa, synthetic construct
MSYYHHHHHHLESTSLYKKAGSGSQRNVETQQKVLELTSDNDRLRNGVEQLSRELDTLRGIFRQLLE

SEQ ID NO: 183 - protein, 84 aa, synthetic construct
MSYYHHHHHHLESTSLYKKAGSEFQRKLETIVQLERELERLTNERERLLRARGEADRTLEVMRQQLTELYRDIFQHLRDESGNS

SEQ ID NO: 184 - protein, 88 aa, synthetic construct
MSYYHHHHHHLESTSLYKKAGSEFKLDTILNLERDVEDLQRDKARLLREKVEFLRSLRQMKQKVQSLYQEVFGRLRDENGRPYYLEII

SEQ ID NO: 185 - protein, 100 aa, synthetic construct
MSYYHHHHHHLESTSLYKKAGSEFFRDIRRRGKNKVAAQNCRKRKLENIVELEQDLDHLKDEKEKLLKEKGENDKSLHLLKKQLSTLYLEVFSMLRDALE

SEQ ID NO: 186 - protein, 104 aa, synthetic construct
MSYYHHHHHHLESTSLYKKAGSEFGDIRRRGKNKVAAQNCRKRKLDIILNLEDDVCNLQAKKETLKREQAQCNKAINIMKQKLHDLYHDIFSRLRDDQGRPVLE

SEQ ID NO: 187 - protein, 89 aa, synthetic construct
MSYYHHHHHHLESTSLYKKAGSEFGCRKRKLDCIQNLESEIEKLQSEKESLLKERDHILSTLGETKQNLTGLCQKVCKEAALSQEQNLE

SEQ ID NO: 188 - protein, 81 aa, synthetic construct
MSYYHHHHHHLESTSLYKKAGSEFRKRKLDCIQNLECEIRKLVCEKEKLLSERNQLKACMGELLDNFSCLSQEVCRDIQLE

SEQ ID NO: 189 - protein, 85 aa, synthetic construct
MSYYHHHHHHLESTSLYKKAGSEFDPQLKREIRLMKNREAARECRRKKKEYVKCLENRVAVLENQNKTLIEELKTLKDLYSNKSV

SEQ ID NO: 190 - protein, 87 aa, synthetic construct
MSYYHHHHHHLESTSLYKKAGSGSKEEATRKRELRLMKNREAARECRRKKKEYVKCLENRVAVLENQNKTLIEELKALKDLYCHKVE

SEQ ID NO: 191 - protein, 86 aa, synthetic construct
MSYYHHHHHHLESTSLYKKAGSGSKEEATRKRELRLMKNREAARECRRKKKEYVKCLENRVAVLENQNKTLIEELKALRDLYCQLE

SEQ ID NO: 192 - protein, 58 aa, synthetic construct
MSYYHHHHHHLESTSLYKKAGSGSEYVKCLESRVAVLEVQNKKLIEELETLKDICSLE

SEQ ID NO: 193 - protein, 86 aa, synthetic construct
MSYYHHHHHHLESTSLYKKAGSGSKEAARKREVRLMKNREAARECRRKKKEYVKCLENRVAVLENQNKTLIEELKALKDLYCHKSD

SEQ ID NO: 194 - protein, 94 aa, synthetic construct
MSYYHHHHHHLESTSLYKKAGSEFEKRRLNDLVLENKLIALGEENATLKAELLSLKLKFGLISSTAYAQEIQKLSNSTAVYFQDYQTSKSNVLE

SEQ ID NO: 195 - protein, 37 aa, synthetic construct
LNDAAIAGRLAALMEENALLKGELKALKLRFGLLPLT

SEQ ID NO: 196 - protein, 102 aa, synthetic construct
MSYYHHHHHHLESTSLYKKAGSEFFRRERNKMAAAKCRNRRRELTDTLQAETDQLEDEKSALQTEIANLLKEKEKLEFILAAHRPACKIPDDLGFPEEMSLE

SEQ ID NO: 197 - protein, 94 aa, synthetic construct
MSYYHHHHHHLESTSLYKKAGSEFNRERNKLAAAKCRNRRRELTDRLQAETDQLEEEKAELESEIAELQKEKERLEFVLVAHKPGCKIPYEALE

SEQ ID NO: 198 - protein, 90 aa, synthetic construct
MSYYHHHHHHLESTSLYKKAGSEFNRRKELTDFLQAETDKLEDEKSGLQREIEELQKQKERLELVLEAHRPICKIPEGAKEGDTGSTSLE

SEQ ID NO: 199 - protein, 90 aa, synthetic construct
MSYYHHHHHHLESTSLYKKAGSEFERNKLAAAKCRNRRRELTEKLQAETEELEEEKSGLQKEIAELQKEKEKLEFMLVAHGPVCKISPLE

SEQ ID NO: 200 - protein, 84 aa, synthetic construct
MSYYHHHHHHLESTSLYKKAGSEFQRKLERIARLEEKVKTLKAQNSELASTANMLREQVAQLKQKVMNHVNSGCQLMLTQQYLE

SEQ ID NO: 201 - protein, 107 aa, synthetic construct
MSYYHHHHHHLESTSLYKKAGSEFNIKVERKRLRNRLAATKCRKRKLERIARLEDKVKTLKAENAGLSSTAGLLREQVAQLKQKVMTHVSNGCQLLLGVKGHAFYLE

SEQ ID NO: 202 - protein, 98 aa, synthetic construct
MSYYHHHHHHLESTSLYKKAGSGSDMDTQERIKAERKRLRNRIAASKCRKRKLERISRLEEKVKTLKSQNTELASTASLLREQVAQLKQKVLSHVNLE

SEQ ID NO: 203 - protein, 90 aa, synthetic construct
MSYYHHHHHHLESTSLYKKAGSEFQRVQQRHVLESEKNQLLQQVDHLKQEISRLVRERDAYKEKYEKLVSSGFRENGSSSDNPSSPEFFM

SEQ ID NO: 204 - protein, 92 aa, synthetic construct
MSYYHHHHHHLESTSLYKKAGSEFQYKRVQQKHHLENEKTQLIQQVEQLKQEVSRLARERDAYKVKCEKLANSGFREAGSTSDSPSSPEFFL

SEQ ID NO: 205 - protein, 46 aa, synthetic construct
QQRHILESEKCQLQSQVEQLKLEVGRLAKERDLYKEKYEKLAGRGG

SEQ ID NO: 206 - protein, 45 aa, synthetic construct
QQRRGLEAERARLAAQLDALRAEVARLARERDLYKARCDRLTSSG

SEQ ID NO: 207 - protein, 77 aa, synthetic construct
MSYYHHHHHHLESTSLYKKAGSGSEYVECLEKKVETFTSENNELWKKVETLENANRTLLQQLQKLQTLVTNKISRLE

SEQ ID NO: 208 - protein, 76 aa, synthetic construct
MSYYHHHHHHLESTSLYKKAGSGSEYIDGLETRMSACTAQNQELQRKVLHLEKQNLSLLEQLKKLQAIVVQSTSLE

SEQ ID NO: 209 - protein, 100 aa, synthetic construct
MSYYHHHHHHLESTSLYKKAGSEFWRRKIRNKRSAQESRRKKKVYVGGLESRVLKYTAQNMELQNKVQLLEEQNLSLLDQLRKLQAMVIEISNKTSSRLE

SEQ ID NO: 210 - protein, 104 aa, synthetic construct
MSYYHHHHHHLESTSLYKKAGSEFWRKIRNKQSAQDSRRRKKEYIDGLESRVAACSAQNQELQKKVQELERHNISLVAQLRQLQTLIAQTSNKAAQTSTCVLLE

SEQ ID NO: 211 - protein, 39 aa, synthetic construct
EYMDSLEDDVESCSTENLELRKKVEVLENTNRASHGELV

SEQ ID NO: 212 - protein, 99 aa, synthetic construct
MSYYHHHHHHLESTSLYKKAGSGSEEQKDEKYWSRRYKNNEAAKRSRDARRLKENQISVRAAFLEKENALLRQEVVAVRQELSHYRAVLSRYQAQHGAL

SEQ ID NO: 213 - protein, 71 aa, synthetic construct
MSYYHHHHHHLESTSLYKKAGSGRLKENQIAIRASFLEKENSALRQEVADLRKELGKCKNILAKYEARHLE

SEQ ID NO: 214 - protein, 92 aa, synthetic construct
MSYYHHHHHHLESTSLYKKAGSEFNWTRRKKNNVAAKRSRDARRLKENQITIRAAFLEKENTALRTEVAELRKEVGKCKTIVSKYETKYGPL

SEQ ID NO: 215 - protein, 115 aa, synthetic construct
MSYYHHHHHHLESTSLYKKAGSEFGVTQKEELEKQKAELQQEVEKLASENASMKLELDALRSKYEALQTFARTVARSPVAPARGPLAAGLGPLVPGKVAATSVITIVKSKTDALE

SEQ ID NO: 216 - protein, 76 aa, synthetic construct
MSYYHHHHHHLESTSLYKKAGSGSTQKEELERQRVELQQEVEKLARENSSMRLELDALRSKYEALQTFARTVARLE

SEQ ID NO: 217 - protein, 53 aa, synthetic construct
CQKEELQKQKSELEREVDKLARENAAMRLELDALRGKCEALQGFARSVAAARG

SEQ ID NO: 218 - protein, 97 aa, synthetic construct
MSYYHHHHHHLESTSLYKKAGSEFQTARDRKKARMSELEQQVVDLEEENQKLLLENQLLREKTHGLVVENQELRQRLGMDALVAEEEAEAKGNEVLE

SEQ ID NO: 219 - protein, 96 aa, synthetic construct
MSYYHHHHHHLESTSLYKKAGSEFPSQNAFRKRKLERLEELEKKEAQLTVTNDQIHILKKENELLHFMLRSLLTERNMPCSSNTQLSCTKWLIRGC

SEQ ID NO: 220 - protein, 78 aa, synthetic construct
MSYYHHHHHHLESTSLYKKAGSEFKRAAQNRSAQKAFRQRREKYIKNLEEKSKLFDGLMKENSELKKMIESLKSKLKE

SEQ ID NO: 221 - protein, 94 aa, synthetic construct
MSYYHHHHHHLESTSLYKKAGSEFEKRTAQNRAAQRAFRERKERKMKELEKKVQSLESIQQQNEVEATFLRDQLITLDSSNTQLSCTKWLIRGC

SEQ ID NO: 222 - protein, 132 aa, synthetic construct
MSYYHHHHHHLESTSLYKKAGFDDEDTPPNPKKEIEFPGGSPGRKRIDSEAKSRRTAQNRAAQRAFRDRKEAKMKSLQERVELLEQKDAQNKTTTDFLLCSLKSLLSEITKYRAKNSDDERILAFLDDLHLE

SEQ ID NO: 223 - protein, 109 aa, synthetic construct
MSYYHHHHHHLESTSLYKKAGFDDEDTPPNPKKEIEFQAKKKAQNRAAQKAFRERKEARMKELQDKLLESERNRQSLLKEIEELRKANTEINAENRLLLRSGNSTCRDL

SEQ ID NO: 224 - protein, 125 aa, synthetic construct
MSYYHHHHHHLESTSLYKKAGFDDEDTPPNPKKEIEFPKRQNRDAQRAYRERKNNKLQVLEETIESLSKVVKNYETKLNRLQNELQAKESENHALKQKLETLTLKQASVPAQDPILQNFSTCRDL

SEQ ID NO: 225 - protein, 121 aa, synthetic construct
MSYYHHHHHHLESTSLYKKAGSEFQQNRDAQRAYRERRTTRIQVLEEKVEMLHNLVDDWQRKYKLLESEFSDTKENLQKSIALNNELQKALPLIVNTPFQQQPENSSNTQLSCTKWLIRGC

SEQ ID NO: 226 - protein, 112 aa, synthetic construct
MSYYHHHHHHLESTSLYKKAGFDDEDTPPNPKKEIEFGIRKKQKNFENMNKLQNLNTQINKLRDRIEQLNKENEFWKAKLNDINEIKSLKLLNDIKRRNMGLDLQRSMNRRY

SEQ ID NO: 227 - protein, 102 aa, synthetic construct
MSYYHHHHHHLESTSLYKKAGFDDEDTPPNPKKEIEFHPAALKRARNTEAARRSRARKLQRMKQLEDKVEELLSKNYHLENEVARLKKLVGDAARPAEIYES

SEQ ID NO: 228 - protein, 106 aa, synthetic construct
MSYYHHHHHHLESTSLYKKAGFDDEDTPPNPKKEIEFDDDEDLLIQSKKSHQKKKLKEKELESSIHELTEIAASLQKRIHTLETENKLLKNLVLSSGETFSTCRDL

SEQ ID NO: 229 - protein, 19 aa, unidentified
AEGRGSLLTCGDVEENPGP

SEQ ID NO: 230 - protein, 18 aa, unidentified
EGRGSLLTCGDVEENPGP

SEQ ID NO: 231 - protein, 21 aa, unidentified
GSGEGRGSLLTCGDVEENPGP

SEQ ID NO: 232 - protein, 63 aa, unidentified
GSGSRVTELLYRMKRAETYCPRPLLAIHPTEARHKQKIVAPVKQLLNFDLLKLAGDVESNPGP

SEQ ID NO: 233 - protein, 19 aa, unidentified
LLCFLLLLLSGDVELNPGP

SEQ ID NO: 234 - protein, 20 aa, unidentified
HHFMFLLLLLAGDIELNPGP

SEQ ID NO: 235 - protein, 20 aa, Saccoglossus kowalevskii
WFLVLLSFILSGDIEVNPGP

SEQ ID NO: 236 - protein, 20 aa, Branchiostoma floridae
KNCAMYMLLLSGDVETNPGP

SEQ ID NO: 237 - protein, 20 aa, Branchiostoma floridae
MVISQLMLKLAGDVEENPGP

SEQ ID NO: 238 - protein, 23 aa, Equine rhinitis A virus
GSGQCTNYALLKLAGDVESNPGP

SEQ ID NO: 239 - protein, 8 aa, synthetic construct
  VARIANT 2: note = Any amino acid
  VARIANT 4: note = Any amino acid
DXEXNPGP

SEQ ID NO: 240 - protein, 174 aa, synthetic construct
MDPDLEIRAAFLRQRNTALRTEVAELEQEVQRLENEVSQYETRYGPLGGGKSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPETG

SEQ ID NO: 241 - protein, 1021 aa, synthetic construct
MDWIWRILFLVGAATGAHSDYKDDDDKMDPDLEIEAAFLERENTALETRVAELRQRVQRLRNRVSQYRTRYGPLGGGKGGGGSGGGGSGGGGSMDPDLEIEAAFLERENTALETRVAELRQRVQRLRNRVSQYRTRYGPLGGGKPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLVGSGATNFSLLKQAGDVEENPGPMDWIWRILFLVGAATGAHSEQKLISEEDLMDPDLEIRAAFLRQRNTALRTEVAELEQEVQRLENEVSQYETRYGPLGGGKGGGGSGGGGSGGGGSMDPDLEIRAAFLRQRNTALRTEVAELEQEVQRLENEVSQYETRYGPLGGGKSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPETGSGATNFSLLKQAGDVEENPGPMVSKGEELFTGVVPILVELDGDVNGHKFSVRGEGEGDATNGKLTLKFICTTGKLPVPWPTLVTTFGYGVACFSRYPDHMKQHDFFKSAMPEGYVQERTISFKDDGTYKTRAEVKFEGDTLVNRIELKGIDFKEDGNILGHKLEYNFNSHYVYITADKQKNCIKANFKIRHNVEDGSVQLADHYQQNTPIGDGPVLLPDNHYLSHQSKLSKDPNEKRDHMVLLEFVTAAGITHGMDELYK

SEQ ID NO: 242 - DNA, 3067 bp, synthetic construct
  source 1..3067: mol_type = other DNA
atggattggatctggcggatcctgttccttgtgggagctgccacaggcgcccacagcgactacaaggacgacgacgacaagatggaccccgacctggaaatcgaggccgccttcctggaaagagagaacacagccctggaaaccagagtggccgagctgagacagagagtgcagcggctgagaaaccgggtgtcccagtacagaaccagatacggacctctcggcggaggaaaaggtggcggaggttctggcggaggcggctctggtggtggtggatctatggatcctgacctcgagattgaagccgcctttctcgagcgcgagaataccgctctcgagacaagagtggctgaactgcggcagagggtccagagactgaggaacagagtgtctcagtaccggacacgctacggccctcttggaggtggaaaacctctggccttcagaacaaaacctgccgctctgggcaaagacacaatcccctggctgggacatctgctcgttggactgtctggcgccttcggcttcatcatcctggtgtacctgctgatcaactgccggaacacaggcccttggctgaagaaagtgctgaagtgcaacacccctgatccgagcaagttctttagccagctgagcagcgagcatggcggcgacgttcagaaatggctgtctagcccatttcctagcagcagcttcagccctggtggactggcccctgagattagccctctggaagtgctggaacgggacaaagtgacccagctgctcctgcagcaggataaggtgccagaacctgccagcctgtccagcaatcacagcctgaccagctgctttaccaaccagggctacttcttcttccatctgcctgacgctctcgagatcgaggcctgccaggtgtacttcacctacgatccctacagcgaggaagatcccgacgaaggcgttgcaggcgctcctacaggatcttctccacagcctctgcagccactgtccggcgaggatgatgcctactgcacctttccaagccgggacgacctgctgctgtttagccctagtctgctcggcggaccttctccaccatctacagctccaggtggaagcggagccggcgaagaaagaatgcctcctagcctgcaagagcgggtgcccagagattgggatcctcaacctctcggacctcctacaccaggcgtgccagacctggtggattttcagcctcctccagagctggtgctgagagaagctggcgaagaagtgccagacgccggacctagagagggcgttagctttccttggagcagacctcctggacagggcgagttcagagccctgaatgctagactgcccctgaacaccgatgcctacctgtctctgcaagaactgcagggacaagaccccacacacctcgttgggtagcggcgccacaaatttcagcctgctgaaacaggccggcgacgtggaagagaaccctggacctatggattggatctggcggatcctgttccttgtgggagctgccacaggcgcccactctgagcagaagctgatctccgaagaggacctgatggaccccgacctggaaatcagagccgccttcctgcggcagagaaacacagccctgagaacagaggtggccgagctggaacaagaggtgcagcggctggaaaacgaggtgtcccagtacgagacaagatacggccctctcggcggaggaaaaggtggcggaggttctggcggaggcggaagtggtggtggcggatctatggatcctgacctcgagattagggccgcctttctgagacagcggaacaccgctctgaggaccgaagtggccgaactcgagcaagaagtccagagactggaaaatgaagtctctcagtacgaaacccgctacggacctcttggaggcggcaaaagcaacaccagcaaagagaaccccttcctgttcgccctggaagccgtggttatcagcgtgggctctatgggcctgatcatcagcctgctgtgcgtgtacttctggctggaacggaccatgcctcggatccccacactgaagaacctggaagatctggtcaccgagtaccacggcaacttctctgcttggagcggcgtgtcaaaaggcctggccgaatctctgcagcccgactactctgagagactgtgcctggtgtctgagatccctcctaaaggcggagccctcggagaaggacctggtgcctctccatgtaaccagcacagcccttattgggcccctccttgctacaccctgaagcctgaaacaggcagcggcgccaccaactttagcctgctgaaacaagccggcgacgtggaagagaatcccggacctatggtgagcaagggcgaggagctgttcaccggggtggtgcccatcctggtcgagctggacggcgacgtaaacggccacaagttcagcgtccgcggcgaaggtgagggagatgccaccaacggcaagctgaccctgaagttcatctgcaccaccggcaagctgcccgtgccctggcccaccctcgtgaccaccttcggctacggcgtggcctgcttcagccgctaccccgaccacatgaagcagcacgacttcttcaagtccgccatgcccgaaggctacgtccaggagcgcaccatctctttcaaggacgacggtacctacaagacccgcgccgaggtgaagttcgagggcgacaccctggtgaaccgcatcgagctgaagggcatcgacttcaaggaggacggcaacatcctggggcacaagctggagtacaacttcaacagccactacgtctatatcacggccgacaagcagaagaactgcatcaaggctaacttcaagatccgccacaacgttgaggacggcagcgtgcagctcgccgaccactaccagcagaacacccccatcggcgacggccccgtgctgctgcccgacaaccactacctgagccatcagtccaagctgagcaaagaccccaacgagaagcgcgatcacatggtcctgctggagttcgtgaccgccgccgggattacacatggcatggacgagctgtacaagtaa

SEQ ID NO: 243 - protein, 930 aa, synthetic construct
MDWIWRILFLVGAATGAHSDYKDDDDKMDPDLEIEAAFLERENTALETRVAELRQRVQRLRNRVSQYRTRYGPLGGGKGGGGSGGGGSGGGGSMDPDLEIEAAFLERENTALETRVAELRQRVQRLRNRVSQYRTRYGPLGGGKYFRTPEINNSSGEMDPILLTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQGSGATNFSLLKQAGDVEENPGPMDWIWRILFLVGAATGAHSEQKLISEEDLMDPDLEIRAAFLRQRNTALRTEVAELEQEVQRLENEVSQYETRYGPLGGGKGGGGSGGGGSGGGGSMDPDLEIRAAFLRQRNTALRTEVAELEQEVQRLENEVSQYETRYGPLGGGKSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPETGSGATNFSLLKQAGDVEENPGPMVSKGEELFTGVVPILVELDGDVNGHKFSVRGEGEGDATNGKLTLKFICTTGKLPVPWPTLVTTFGYGVACFSRYPDHMKQHDFFKSAMPEGYVQERTISFKDDGTYKTRAEVKFEGDTLVNRIELKGIDFKEDGNILGHKLEYNFNSHYVYITADKQKNCIKANFKIRHNVEDGSVQLADHYQQNTPIGDGPVLLPDNHYLSHQSKLSKDPNEKRDHMVLLEFVTAAGITHGMDELYK

SEQ ID NO: 244 - DNA, 2794 bp, synthetic construct
  source 1..2794: mol_type = other DNA
atggattggatctggcggatcctgttccttgtgggagctgccacaggcgcccacagcgactacaaggacgacgacgacaagatggaccccgacctggaaatcgaggccgccttcctggaaagagagaacacagccctggaaaccagagtggccgagctgagacagagagtgcagcggctgagaaaccgggtgtcccagtacagaaccagatacggacctctcggcggaggaaaaggtggcggaggttctggcggaggcggctctggtggtggtggatctatggatcctgacctcgagattgaagccgcctttctcgagcgcgagaataccgctctcgagacaagagtggctgaactgcggcagagggtccagagactgaggaacagagtgtctcagtaccggacacgctacggccctcttggaggtggaaaatacttcagaacccctgagatcaacaacagcagcggcgagatggatcccatcctgctgacaatcagcatcctgagctttttcagcgtggccctgctggtcatcctggcctgtgtgctgtggaagaagcggatcaagcccatcgtgtggcccagcctgcctgaccacaagaaaaccctggaacacctgtgcaagaagccccggaagaacctgaacgtgtccttcaatcccgagagcttcctggactgtcagatccacagagtggacgacatccaggccagggacgaagtggaaggctttctgcaggacacattccctcagcagctggaagagagcgagaagcagagacttggaggcgacgtgcagagccctaattgcccttctgaggacgtggtcatcaccccagagagcttcggcagagatagcagcctgacatgtctggccggcaatgtgtccgcctgtgatgcccctatcctgagcagcagcagaagcctggattgcagagagagcggcaagaacggccctcacgtgtaccaggatctgctcctgtctctgggcaccaccaatagcacactgcctccaccattcagcctgcagagcggcatcctgacactgaaccctgttgctcagggccagccaatcctgacaagcctgggcagcaatcaagaagaggcctacgtcaccatgagcagcttctaccagaatcaagggtagcggcgccacaaatttcagcctgctgaaacaggccggcgacgtggaagagaaccctggacctatggattggatctggcggatcctgttccttgtgggagctgccacaggcgcccactctgagcagaagctgatctccgaagaggacctgatggaccccgacctggaaatcagagccgccttcctgcggcagagaaacacagccctgagaacagaggtggccgagctggaacaagaggtgcagcggctggaaaacgaggtgtcccagtacgagacaagatacggccctctcggcggaggaaaaggtggcggaggttctggcggaggcggaagtggtggtggcggatctatggatcctgacctcgagattagggccgcctttctgagacagcggaacaccgctctgaggaccgaagtggccgaactcgagcaagaagtccagagactggaaaatgaagtctctcagtacgaaacccgctacggacctcttggaggcggcaaaagcaacaccagcaaagagaaccccttcctgttcgccctggaagccgtggttatcagcgtgggctctatgggcctgatcatcagcctgctgtgcgtgtacttctggctggaacggaccatgcctcggatccccacactgaagaacctggaagatctggtcaccgagtaccacggcaacttctctgcttggagcggcgtgtcaaaaggcctggccgaatctctgcagcccgactactctgagagactgtgcctggtgtctgagatccctcctaaaggcggagccctcggagaaggacctggtgcctctccatgtaaccagcacagcccttattgggcccctccttgctacaccctgaagcctgaaacaggcagcggcgccaccaactttagcctgctgaaacaagccggcgacgtggaagagaatcccggacctatggtgagcaagggcgaggagctgttcaccggggtggtgcccatcctggtcgagctggacggcgacgtaaacggccacaagttcagcgtccgcggcgaaggtgagggagatgccaccaacggcaagctgaccctgaagttcatctgcaccaccggcaagctgcccgtgccctggcccaccctcgtgaccaccttcggctacggcgtggcctgcttcagccgctaccccgaccacatgaagcagcacgacttcttcaagtccgccatgcccgaaggctacgtccaggagcgcaccatctctttcaaggacgacggtacctacaagacccgcgccgaggtgaagttcgagggcgacaccctggtgaaccgcatcgagctgaagggcatcgacttcaaggaggacggcaacatcctggggcacaagctggagtacaacttcaacagccactacgtctatatcacggccgacaagcagaagaactgcatcaaggctaacttcaagatccgccacaacgttgaggacggcagcgtgcagctcgccgaccactaccagcagaacacccccatcggcgacggccccgtgctgctgcccgacaaccactacctgagccatcagtccaagctgagcaaagaccccaacgagaagcgcgatcacatggtcctgctggagttcgtgaccgccgccgggattacacatggcatggacgagctgtacaagtaa

SEQ ID NO: 245 - protein, 1016 aa, synthetic construct
MDWIWRILFLVGAATGAHSDYKDDDDKMDPDLEIEAAFLERENTALETRVAELRQRVQRLRNRVSQYRTRYGPLGGGKGGGGSGGGGSGGGGSMDPDLEIEAAFLERENTALETRVAELRQRVQRLRNRVSQYRTRYGPLGGGKSDPVIFQTQSEELKEGWNPHLLLLLLLVIVFIPAFWSLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQASGSGATNFSLLKQAGDVEENPGPMDWIWRILFLVGAATGAHSEQKLISEEDLMDPDLEIRAAFLRQRNTALRTEVAELEQEVQRLENEVSQYETRYGPLGGGKGGGGSGGGGSGGGGSMDPDLEIRAAFLRQRNTALRTEVAELEQEVQRLENEVSQYETRYGPLGGGKSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPETGSGATNFSLLKQAGDVEENPGPMVSKGEELFTGVVPILVELDGDVNGHKFSVRGEGEGDATNGKLTLKFICTTGKLPVPWPTLVTTFGYGVACFSRYPDHMKQHDFFKSAMPEGYVQERTISFKDDGTYKTRAEVKFEGDTLVNRIELKGIDFKEDGNILGHKLEYNFNSHYVYITADKQKNCIKANFKIRHNVEDGSVQLADHYQQNTPIGDGPVLLPDNHYLSHQSKLSKDPNEKRDHMVLLEFVTAAGITHGMDELYK

SEQ ID NO: 246 - DNA, 3051 bp, synthetic construct
  source 1..3051: mol_type = other DNA
atggattggatctggcggatcctgttccttgtgggagctgccacaggcgcccacagcgactacaaggacgacgacgacaagatggaccccgacctggaaatcgaggccgccttcctggaaagagagaacacagccctggaaaccagagtggccgagctgagacagagagtgcagcggctgagaaaccgggtgtcccagtacagaaccagatacggacctctcggcggaggaaaaggtggcggaggttctggcggaggcggctctggtggtggtggatctatggatcctgacctcgagattgaagccgcctttctcgagcgcgagaataccgctctcgagacaagagtggctgaactgcggcagagggtccagagactgaggaacagagtgtctcagtaccggacacgctacggccctcttggaggtggaaaaagcgaccccgtgatctttcagacccagagcgaggaactgaaagaaggctggaaccctcacctcctgctgttgctgctgctggtcatcgtgttcatccccgccttttggagcctgaaaacacaccctctgtggcggctgtggaagaaaatctgggccgtgccatctcctgagcggttcttcatgcctctgtacaagggctgcagcggcgacttcaagaaatgggtcggagccccttttaccggcagctctctggaacttggaccttggagccctgaggtgcccagcacactggaagtgtacagctgtcaccctcctagaagccccgccaagagactgcagctgacagagctgcaagagcctgccgagctggtggaatctgatggcgtgcccaagcctagcttctggcccacagctcagaatagcggcggctctgcctacagcgaggaaagagatagaccctacggcctggtgtccatcgacaccgtgacagtgctggatgccgagggaccttgtacctggccttgtagctgcgaggacgatggctaccctgctctggatctggacgctggccttgagccttctccaggactggaagatcctctgctggacgccggaacaaccgtgctgtcttgtggctgtgtgtctgccggatctcctggacttggaggccctctgggaagcctgctggatagactgaaacctcctctggccgacggcgaagattgggctggtggacttccttggggcggaagatctccaggcggagtgtctgagtctgaagccggttctccactggccggcctggacatggataccttcgattctggcttcgtgggcagcgactgtagcagccctgtggaatgcgacttcacaagccctggcgacgagggcccacctagaagctatctgagacagtgggtcgtgatccctccacctctgtctagtcctggacctcaggcttctggtagcggcgccacaaatttcagcctgctgaaacaggccggcgacgtggaagagaaccctggacctatggattggatctggcggatcctgttccttgtgggagctgccacaggcgcccactctgagcagaagctgatctccgaagaggacctgatggaccccgacctggaaatcagagccgccttcctgcggcagagaaacacagccctgagaacagaggtggccgagctggaacaagaggtgcagcggctggaaaacgaggtgtcccagtacgagacaagatacggccctctcggcggaggaaaaggtggcggaggttctggcggaggcggaagtggtggtggcggatctatggatcctgacctcgagattagggccgcctttctgagacagcggaacaccgctctgaggaccgaagtggccgaactcgagcaagaagtccagagactggaaaatgaagtctctcagtacgaaacccgctacggacctcttggaggcggcaaaagcaacaccagcaaagagaaccccttcctgttcgccctggaagccgtggttatcagcgtgggctctatgggcctgatcatcagcctgctgtgcgtgtacttctggctggaacggaccatgcctcggatccccacactgaagaacctggaagatctggtcaccgagtaccacggcaacttctctgcttggagcggcgtgtcaaaaggcctggccgaatctctgcagcccgactactctgagagactgtgcctggtgtctgagatccctcctaaaggcggagccctcggagaaggacctggtgcctctccatgtaaccagcacagcccttattgggcccctccttgctacaccctgaagcctgaaacaggcagcggcgccaccaactttagcctgctgaaacaagccggcgacgtggaagagaatcccggacctatggtgagcaagggcgaggagctgttcaccggggtggtgcccatcctggtcgagctggacggcgacgtaaacggccacaagttcagcgtccgcggcgaaggtgagggagatgccaccaacggcaagctgaccctgaagttcatctgcaccaccggcaagctgcccgtgccctggcccaccctcgtgaccaccttcggctacggcgtggcctgcttcagccgctaccccgaccacatgaagcagcacgacttcttcaagtccgccatgcccgaaggctacgtccaggagcgcaccatctctttcaaggacgacggtacctacaagacccgcgccgaggtgaagttcgagggcgacaccctggtgaaccgcatcgagctgaagggcatcgacttcaaggaggacggcaacatcctggggcacaagctggagtacaacttcaacagccactacgtctatatcacggccgacaagcagaagaactgcatcaaggctaacttcaagatccgccacaacgttgaggacggcagcgtgcagctcgccgaccactaccagcagaacacccccatcggcgacggccccgtgctgctgcccgacaaccactacctgagccatcagtccaagctgagcaaagaccccaacgagaagcgcgatcacatggtcctgctggagttcgtgaccgccgccgggattacacatggcatggacgagctgtacaagtaa

SEQ ID NO: 247 - protein, 961 aa, synthetic construct
MDWIWRILFLVGAATGAHSDYKDDDDKMDPDLEIEAAFLERENTALETRVAELRQRVQRLRNRVSQYRTRYGPLGGGKGGGGSGGGGSGGGGSMDPDLEIEAAFLERENTALETRVAELRQRVQRLRNRVSQYRTRYGPLGGGKAPQRQGPLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGSGATNFSLLKQAGDVEENPGPMDWIWRILFLVGAATGAHSEQKLISEEDLMDPDLEIRAAFLRQRNTALRTEVAELEQEVQRLENEVSQYETRYGPLGGGKGGGGSGGGGSGGGGSMDPDLEIRAAFLRQRNTALRTEVAELEQEVQRLENEVSQYETRYGPLGGGKSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPETGSGATNFSLLKQAGDVEENPGPMVSKGEELFTGVVPILVELDGDVNGHKFSVRGEGEGDATNGKLTLKFICTTGKLPVPWPTLVTTFGYGVACFSRYPDHMKQHDFFKSAMPEGYVQERTISFKDDGTYKTRAEVKFEGDTLVNRIELKGIDFKEDGNILGHKLEYNFNSHYVYITADKQKNCIKANFKIRHNVEDGSVQLADHYQQNTPIGDGPVLLPDNHYLSHQSKLSKDPNEKRDHMVLLEFVTAAGITHGMDELYK

SEQ ID NO: 248 - DNA, 2886 bp, synthetic construct
  source 1..2886: mol_type = other DNA
atggattggatctggcggatcctgttccttgtgggagctgccacaggcgcccacagcgactacaaggacgacgacgacaagatggaccccgacctggaaatcgaggccgccttcctggaaagagagaacacagccctggaaaccagagtggccgagctgagacagagagtgcagcggctgagaaaccgggtgtcccagtacagaaccagatacggacctctcggcggaggaaaaggtggcggaggttctggcggaggcggctctggtggtggtggatctatggatcctgacctcgagattgaagccgcctttctcgagcgcgagaataccgctctcgagacaagagtggctgaactgcggcagagggtccagagactgaggaacagagtgtctcagtaccggacacgctacggccctcttggaggtggaaaagctcctcagagacagggacctctgattcctccttggggctggcctggaaataccctggtggccgtgtccatcttcctgctgctgaccggaccaacctacctgctgttcaagctgagccccagagtgaagcggatcttctaccagaacgtgccctctcctgccatgttcttccagcctctgtacagcgtgcacaacggcaacttccagacatggatgggagcacatggtgccggcgtgctgctgtctcaagattgtgctggaacaccccagggcgctctggaaccttgtgtgcaagaagccaccgctctgctgacatgcggacctgccagaccttggaaaagcgtggccctggaagaggaacaagaaggccctggcacaagactgcccggcaatctgtctagcgaggatgtgctgcctgccggctgtactgaatggcgagttcagaccctggcctacctgcctcaagaagattgggcccctaccagcctgaccagacctgctcctcctgatagcgagggcagcagaagcagcagcagctccagctcctccaacaacaacaattactgtgccctgggctgctacggcggctggcatctttctgctctgcctggcaacacacagagcagcggccctattcctgctctggcctgtggcctgagctgtgatcaccagggactcgagacacagcagggcgttgcatgggttctcgccggccattgtcaaagacctggcctgcacgaagatctgcagggcatgctgctgcctagcgtgctgtctaaggccagaagctggaccttcggtagcggcgccacaaatttcagcctgctgaaacaggccggcgacgtggaagagaaccctggacctatggattggatctggcggatcctgttccttgtgggagctgccacaggcgcccactctgagcagaagctgatctccgaagaggacctgatggaccccgacctggaaatcagagccgccttcctgcggcagagaaacacagccctgagaacagaggtggccgagctggaacaagaggtgcagcggctggaaaacgaggtgtcccagtacgagacaagatacggccctctcggcggaggaaaaggtggcggaggttctggcggaggcggaagtggtggtggcggatctatggatcctgacctcgagattagggccgcctttctgagacagcggaacaccgctctgaggaccgaagtggccgaactcgagcaagaagtccagagactggaaaatgaagtctctcagtacgaaacccgctacggacctcttggaggcggcaaaagcaacaccagcaaagagaaccccttcctgttcgccctggaagccgtggttatcagcgtgggctctatgggcctgatcatcagcctgctgtgcgtgtacttctggctggaacggaccatgcctcggatccccacactgaagaacctggaagatctggtcaccgagtaccacggcaacttctctgcttggagcggcgtgtcaaaaggcctggccgaatctctgcagcccgactactctgagagactgtgcctggtgtctgagatccctcctaaaggcggagccctcggagaaggacctggtgcctctccatgtaaccagcacagcccttattgggcccctccttgctacaccctgaagcctgaaacaggcagcggcgccaccaactttagcctgctgaaacaagccggcgacgtggaagagaatcccggacctatggtgagcaagggcgaggagctgttcaccggggtggtgcccatcctggtcgagctggacggcgacgtaaacggccacaagttcagcgtccgcggcgaaggtgagggagatgccaccaacggcaagctgaccctgaagttcatctgcaccaccggcaagctgcccgtgccctggcccaccctcgtgaccaccttcggctacggcgtggcctgcttcagccgctaccccgaccacatgaagcagcacgacttcttcaagtccgccatgcccgaaggctacgtccaggagcgcaccatctctttcaaggacgacggtacctacaagacccgcgccgaggtgaagttcgagggcgacaccctggtgaaccgcatcgagctgaagggcatcgacttcaaggaggacggcaacatcctggggcacaagctggagtacaacttcaacagccactacgtctatatcacggccgacaagcagaagaactgcatcaaggctaacttcaagatccgccacaacgttgaggacggcagcgtgcagctcgccgaccactaccagcagaacacccccatcggcgacggccccgtgctgctgcccgacaaccactacctgagccatcagtccaagctgagcaaagaccccaacgagaagcgcgatcacatggtcctgctggagttcgtgaccgccgccgggattacacatggcatggacgagctgtacaagtaa

SEQ ID NO: 249 - protein, 1303 aa, synthetic construct
MDWIWRILFLVGAATGAHSDYKDDDDKMDPDLEIEAAFLERENTALETRVAELRQRVQRLRNRVSQYRTRYGPLGGGKGGGGSGGGGSGGGGSMDPDLEIEAAFLERENTALETRVAELRQRVQRLRNRVSQYRTRYGPLGGGKSTKWHNSYREPFEQHLLLGVSVSCIVILAVCLLCYVSITKIKKEWWDQIPNPARSRLVAIIIQDAQGSQWEKRSRGQEPAKCPHWKNCLTKLLPCFLEHNMKRDEDPHKAAKEMPFQGSGKSAWCPVEISKTVLWPESISVVRCVELFEAPVECEEEEEVEEEKGSFCASPESSRDDFQEGREGIVARLTESLFLDLLGEENGGFCQQDMGESCLLPPSGSTSAHMPWDEFPSAGPKEAPPWGKEQPLHLEPSPPASPTQSPDNLTCTETPLVIAGNPAYRSFSNSLSQSPCPRELGPDPLLARHLEEVEPEMPCVPQLSEPTTVPQPEPETWEQILRRNVLQHGAAAAPVSAPTSGYQEFVHAVEQGGTQASAVVGLGPPGEAGYKAFSSLLASSAVSPEKCGFGASSGEEGYKPFQDLIPGCPGDPAPVPVPLFTFGLDREPPRSPQSSHLPSSSPEHLGLEPGEKVEDMPKPPLPQEQATDPLVDSLGSGIVYSALTCHLCGHLKQCHGQEDGGQTPVMASPCCGCCCGDRSSPPTTPLRAPDPSPGGVPLEASLCPASLAPSGISEKSKSSSSFHPAPGNAQSSSQTPKIVNFVSVGPTYMRVSGSGATNFSLLKQAGDVEENPGPMDWIWRILFLVGAATGAHSEQKLISEEDLMDPDLEIRAAFLRQRNTALRTEVAELEQEVQRLENEVSQYETRYGPLGGGKGGGGSGGGGSGGGGSMDPDLEIRAAFLRQRNTALRTEVAELEQEVQRLENEVSQYETRYGPLGGGKSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPETGSGATNFSLLKQAGDVEENPGPMVSKGEELFTGVVPILVELDGDVNGHKFSVRGEGEGDATNGKLTLKFICTTGKLPVPWPTLVTTFGYGVACFSRYPDHMKQHDFFKSAMPEGYVQERTISFKDDGTYKTRAEVKFEGDTLVNRIELKGIDFKEDGNILGHKLEYNFNSHYVYITADKQKNCIKANFKIRHNVEDGSVQLADHYQQNTPIGDGPVLLPDNHYLSHQSKLSKDPNEKRDHMVLLEFVTAAGITHGMDELYK

SEQ ID NO: 250 - DNA, 3912 bp, synthetic construct
  source 1..3912: mol_type = other DNA
atggattggatctggcggatcctgttccttgtgggagctgccacaggcgcccacagcgactacaaggacgacgacgacaagatggaccccgacctggaaatcgaggccgccttcctggaaagagagaacacagccctggaaaccagagtggccgagctgagacagagagtgcagcggctgagaaaccgggtgtcccagtacagaaccagatacggacctctcggcggaggaaaaggtggcggaggttctggcggaggcggctctggtggtggtggatctatggatcctgacctcgagattgaagccgcctttctcgagcgcgagaataccgctctcgagacaagagtggctgaactgcggcagagggtccagagactgaggaacagagtgtctcagtaccggacacgctacggccctcttggaggtggaaaaagcaccaagtggcacaacagctacagggagcccttcgagcagcacctgctgctgggcgtgagcgtgagctgcatcgtgatcctggccgtgtgcctgctgtgctacgtgagcatcaccaagatcaagaaggagtggtgggaccagatccccaaccccgccaggagcaggctggtggccatcatcatccaggacgcccagggcagccagtgggagaagaggagcaggggccaggagcccgccaagtgcccccactggaagaactgcctgaccaagctgctgccctgcttcctggagcacaacatgaagagggacgaggacccccacaaggccgccaaggagatgcccttccagggcagcggcaagagcgcctggtgccccgtggagatcagcaagaccgtgctgtggcccgagagcatcagcgtggtgaggtgcgtggagctgttcgaggcccccgtggagtgcgaggaggaggaggaggtggaggaggagaagggcagcttctgcgccagccccgagagcagcagggacgacttccaggagggcagggagggcatcgtggccaggctgaccgagagcctgttcctggacctgctgggcgaggagaacggcggcttctgccagcaggacatgggcgagagctgcctgctgccccccagcggcagcaccagcgcccacatgccctgggacgagttccccagcgccggccccaaggaggcccccccctggggcaaggagcagcccctgcacctggagcccagcccccccgccagccccacccagagccccgacaacctgacctgcaccgagacccccctggtgatcgccggcaaccccgcctacaggagcttcagcaacagcctgagccagagcccctgccccagggagctgggccccgaccccctgctggccaggcacctggaggaggtggagcccgagatgccctgcgtgccccagctgagcgagcccaccaccgtgccccagcccgagcccgagacctgggagcagatcctgaggaggaacgtgctgcagcacggcgccgccgccgcccccgtgagcgcccccaccagcggctaccaggagttcgtgcacgccgtggagcagggcggcacccaggccagcgccgtggtgggcctgggcccccccggcgaggccggctacaaggccttcagcagcctgctggccagcagcgccgtgagccccgagaagtgcggcttcggcgccagcagcggcgaggagggctacaagcccttccaggacctgatccccggctgccccggcgaccccgcccccgtgcccgtgcccctgttcaccttcggcctggacagggagccccccaggagcccccagagcagccacctgcccagcagcagccccgagcacctgggcctggagcccggcgagaaggtggaggacatgcccaagccccccctgccccaggagcaggccaccgaccccctggtggacagcctgggcagcggcatcgtgtacagcgccctgacctgccacctgtgcggccacctgaagcagtgccacggccaggaggacggcggccagacccccgtgatggccagcccctgctgcggctgctgctgcggcgacaggagcagcccccccaccacccccctgagggcccccgaccccagccccggcggcgtgcccctggaggccagcctgtgccccgccagcctggcccccagcggcatcagcgagaagagcaagagcagcagcagcttccaccccgcccccggcaacgcccagagcagcagccagacccccaagatcgtgaacttcgtgagcgtgggccccacctacatgagggtgagcggtagcggcgccacaaatttcagcctgctgaaacaggccggcgacgtggaagagaaccctggacctatggattggatctggcggatcctgttccttgtgggagctgccacaggcgcccactctgagcagaagctgatctccgaagaggacctgatggaccccgacctggaaatcagagccgccttcctgcggcagagaaacacagccctgagaacagaggtggccgagctggaacaagaggtgcagcggctggaaaacgaggtgtcccagtacgagacaagatacggccctctcggcggaggaaaaggtggcggaggttctggcggaggcggaagtggtggtggcggatctatggatcctgacctcgagattagggccgcctttctgagacagcggaacaccgctctgaggaccgaagtggccgaactcgagcaagaagtccagagactggaaaatgaagtctctcagtacgaaacccgctacggacctcttggaggcggcaaaagcaacaccagcaaagagaaccccttcctgttcgccctggaagccgtggttatcagcgtgggctctatgggcctgatcatcagcctgctgtgcgtgtacttctggctggaacggaccatgcctcggatccccacactgaagaacctggaagatctggtcaccgagtaccacggcaacttctctgcttggagcggcgtgtcaaaaggcctggccgaatctctgcagcccgactactctgagagactgtgcctggtgtctgagatccctcctaaaggcggagccctcggagaaggacctggtgcctctccatgtaaccagcacagcccttattgggcccctccttgctacaccctgaagcctgaaacaggcagcggcgccaccaactttagcctgctgaaacaagccggcgacgtggaagagaatcccggacctatggtgagcaagggcgaggagctgttcaccggggtggtgcccatcctggtcgagctggacggcgacgtaaacggccacaagttcagcgtccgcggcgaaggtgagggagatgccaccaacggcaagctgaccctgaagttcatctgcaccaccggcaagctgcccgtgccctggcccaccctcgtgaccaccttcggctacggcgtggcctgcttcagccgctaccccgaccacatgaagcagcacgacttcttcaagtccgccatgcccgaaggctacgtccaggagcgcaccatctctttcaaggacgacggtacctacaagacccgcgccgaggtgaagttcgagggcgacaccctggtgaaccgcatcgagctgaagggcatcgacttcaaggaggacggcaacatcctggggcacaagctggagtacaacttcaacagccactacgtctatatcacggccgacaagcagaagaactgcatcaaggctaacttcaagatccgccacaacgttgaggacggcagcgtgcagctcgccgaccactaccagcagaacacccccatcggcgacggccccgtgctgctgcccgacaaccactacctgagccatcagtccaagctgagcaaagaccccaacgagaagcgcgatcacatggtcctgctggagttcgtgaccgccgccgggattacacatggcatggacgagctgtacaagtaa

SEQ ID NO: 251 - protein, 960 aa, synthetic construct
MDWIWRILFLVGAATGAHSDYKDDDDKMDPDLEIEAAFLERENTALETRVAELRQRVQRLRNRVSQYRTRYGPLGGGKGGGGSGGGGSGGGGSMDPDLEIEAAFLERENTALETRVAELRQRVQRLRNRVSQYRTRYGPLGGGKWSQPQRFSIEVQVSDWLIFFASLGSFLSILLVGVLGYLGLNRAARHLCPPLPTPCASSAIEFPGGKETWQWINPVDFQEEASLQEALVVEMSWDKGERTEPLEKTELPEGAPELALDTELSLEDGDRCKAKMGSGATNFSLLKQAGDVEENPGPMDWIWRILFLVGAATGAHSEQKLISEEDLMDPDLEIRAAFLRQRNTALRTEVAELEQEVQRLENEVSQYETRYGPLGGGKGGGGSGGGGSGGGGSMDPDLEIRAAFLRQRNTALRTEVAELEQEVQRLENEVSQYETRYGPLGGGKSHGNEREFCLQGKANWMAFVAPSICIAIIMVGIFSTHYFQQKVFVLLAALRPQWCSREIPDPANSTCAKKYPIAEEKTQLPLDRLLIDWPTPEDPEPLVISEVLHQVTPVFRHPPCSNWPQREKGIQGHQASEKDMMHSASSPPPPRALQAESRQLVDLYKVLESRGSDPKPENPACPWTVLPAGDLPTHDGYLPSNIDDLPSHEAPLADSLEELEPQHISLSVFPSSSLHPLTFSCGDKLTLDQLKMRCDSLMLGSGATNFSLLKQAGDVEENPGPMVSKGEELFTGVVPILVELDGDVNGHKFSVRGEGEGDATNGKLTLKFICTTGKLPVPWPTLVTTFGYGVACFSRYPDHMKQHDFFKSAMPEGYVQERTISFKDDGTYKTRAEVKFEGDTLVNRIELKGIDFKEDGNILGHKLEYNFNSHYVYITADKQKNCIKANFKIRHNVEDGSVQLADHYQQNTPIGDGPVLLPDNHYLSHQSKLSKDPNEKRDHMVLLEFVTAAGITHGMDELYK

SEQ ID NO: 252 - DNA, 2883 bp, synthetic construct
  source 1..2883: mol_type = other DNA
atggattggatctggcggatcctgttccttgtgggagctgccacaggcgcccacagcgactacaaggacgacgacgacaagatggaccccgacctggaaatcgaggccgccttcctggaaagagagaacacagccctggaaaccagagtggccgagctgagacagagagtgcagcggctgagaaaccgggtgtcccagtacagaaccagatacggacctctcggcggaggaaaaggtggcggaggttctggcggaggcggctctggtggtggtggatctatggatcctgacctcgagattgaagccgcctttctcgagcgcgagaataccgctctcgagacaagagtggctgaactgcggcagagggtccagagactgaggaacagagtgtctcagtaccggacacgctacggccctcttggaggtggaaaatggagccagccccagaggttcagcatcgaggtgcaggtgagcgactggctgatcttcttcgccagcctgggcagcttcctgagcatcctgctggtgggcgtgctgggctacctgggcctgaacagggccgccaggcacctgtgcccccccctgcccaccccctgcgccagcagcgccatcgagttccccggcggcaaggagacctggcagtggatcaaccccgtggacttccaggaggaggccagcctgcaggaggccctggtggtggagatgagctgggacaagggcgagaggaccgagcccctggagaagaccgagctgcccgagggcgcccccgagctggccctggacaccgagctgagcctggaggacggcgacaggtgcaaggccaagatgggtagcggcgccacaaatttcagcctgctgaaacaggccggcgacgtggaagagaaccctggacctatggattggatctggcggatcctgttccttgtgggagctgccacaggcgcccactctgagcagaagctgatctccgaagaggacctgatggaccccgacctggaaatcagagccgccttcctgcggcagagaaacacagccctgagaacagaggtggccgagctggaacaagaggtgcagcggctggaaaacgaggtgtcccagtacgagacaagatacggccctctcggcggaggaaaaggtggcggaggttctggcggaggcggaagtggtggtggcggatctatggatcctgacctcgagattagggccgcctttctgagacagcggaacaccgctctgaggaccgaagtggccgaactcgagcaagaagtccagagactggaaaatgaagtctctcagtacgaaacccgctacggacctcttggaggcggcaaaagccacggcaacgagagggagttctgcctgcagggcaaggccaactggatggccttcgtggcccccagcatctgcatcgccatcatcatggtgggcatcttcagcacccactacttccagcagaaggtgttcgtgctgctggccgccctgaggccccagtggtgcagcagggagatccccgaccccgccaacagcacctgcgccaagaagtaccccatcgccgaggagaagacccagctgcccctggacaggctgctgatcgactggcccacccccgaggaccccgagcccctggtgatcagcgaggtgctgcaccaggtgacccccgtgttcaggcaccccccctgcagcaactggccccagagggagaagggcatccagggccaccaggccagcgagaaggacatgatgcacagcgccagcagcccccccccccccagggccctgcaggccgagagcaggcagctggtggacctgtacaaggtgctggagagcaggggcagcgaccccaagcccgagaaccccgcctgcccctggaccgtgctgcccgccggcgacctgcccacccacgacggctacctgcccagcaacatcgacgacctgcccagccacgaggcccccctggccgacagcctggaggagctggagccccagcacatcagcctgagcgtgttccccagcagcagcctgcaccccctgaccttcagctgcggcgacaagctgaccctggaccagctgaagatgaggtgcgacagcctgatgctgggcagcggcgccaccaactttagcctgctgaaacaagccggcgacgtggaagagaatcccggacctatggtgagcaagggcgaggagctgttcaccggggtggtgcccatcctggtcgagctggacggcgacgtaaacggccacaagttcagcgtccgcggcgaaggtgagggagatgccaccaacggcaagctgaccctgaagttcatctgcaccaccggcaagctgcccgtgccctggcccaccctcgtgaccaccttcggctacggcgtggcctgcttcagccgctaccccgaccacatgaagcagcacgacttcttcaagtccgccatgcccgaaggctacgtccaggagcgcaccatctctttcaaggacgacggtacctacaagacccgcgccgaggtgaagttcgagggcgacaccctggtgaaccgcatcgagctgaagggcatcgacttcaaggaggacggcaacatcctggggcacaagctggagtacaacttcaacagccactacgtctatatcacggccgacaagcagaagaactgcatcaaggctaacttcaagatccgccacaacgttgaggacggcagcgtgcagctcgccgaccactaccagcagaacacccccatcggcgacggccccgtgctgctgcccgacaaccactacctgagccatcagtccaagctgagcaaagaccccaacgagaagcgcgatcacatggtcctgctggagttcgtgaccgccgccgggattacacatggcatggacgagctgtacaagtaa

SEQ ID NO: 253 - protein, 994 aa, synthetic construct
MDWIWRILFLVGAATGAHSDYKDDDDKMDPDLEIEAAFLERENTALETRVAELRQRVQRLRNRVSQYRTRYGPLGGGKGGGGSGGGGSGGGGSMDPDLEIEAAFLERENTALETRVAELRQRVQRLRNRVSQYRTRYGPLGGGKWSQPQRFSIEVQVSDWLIFFASLGSFLSILLVGVLGYLGLNRAARHLCPPLPTPCASSAIEFPGGKETWQWINPVDFQEEASLQEALVVEMSWDKGERTEPLEKTELPEGAPELALDTELSLEDGDRCKAKMGSGATNFSLLKQAGDVEENPGPMDWIWRILFLVGAATGAHSEQKLISEEDLMDPDLEIRAAFLRQRNTALRTEVAELEQEVQRLENEVSQYETRYGPLGGGKGGGGSGGGGSGGGGSMDPDLEIRAAFLRQRNTALRTEVAELEQEVQRLENEVSQYETRYGPLGGGKISTGHLTSDNRGDIGLLLGMIVFAVMLSILSLIGIFNRSFRTGIKRRILLLIPKWLYEDIPNMKNSNVVKMLQENSELMNNNSSEQVLYVDPMITEIKEIFIPEHKPTDYKKENTGPLETRDYPQNSLFDNTTVVYIPDLNTGYKPQISNFLPEGSHLSNNNEITSLTLKPPVDSLDSGNNPRLQKHPNFAFSVSSVNSLSNTIFLGELSLILNQGECSSPDIQNSVEEETTMLLENDSPSETIPEQTLLPDEFVSCLGIVNEELPSINTYFPQNILESHFNRISLLEKGSGATNFSLLKQAGDVEENPGPMVSKGEELFTGVVPILVELDGDVNGHKFSVRGEGEGDATNGKLTLKFICTTGKLPVPWPTLVTTFGYGVACFSRYPDHMKQHDFFKSAMPEGYVQERTISFKDDGTYKTRAEVKFEGDTLVNRIELKGIDFKEDGNILGHKLEYNFNSHYVYITADKQKNCIKANFKIRHNVEDGSVQLADHYQQNTPIGDGPVLLPDNHYLSHQSKLSKDPNEKRDHMVLLEFVTAAGITHGMDELYK

SEQ ID NO: 254 - DNA, 2985 bp, synthetic construct
  source 1..2985: mol_type = other DNA
atggattggatctggcggatcctgttccttgtgggagctgccacaggcgcccacagcgactacaaggacgacgacgacaagatggaccccgacctggaaatcgaggccgccttcctggaaagagagaacacagccctggaaaccagagtggccgagctgagacagagagtgcagcggctgagaaaccgggtgtcccagtacagaaccagatacggacctctcggcggaggaaaaggtggcggaggttctggcggaggcggctctggtggtggtggatctatggatcctgacctcgagattgaagccgcctttctcgagcgcgagaataccgctctcgagacaagagtggctgaactgcggcagagggtccagagactgaggaacagagtgtctcagtaccggacacgctacggccctcttggaggtggaaaatggagccagccccagaggttcagcatcgaggtgcaggtgagcgactggctgatcttcttcgccagcctgggcagcttcctgagcatcctgctggtgggcgtgctgggctacctgggcctgaacagggccgccaggcacctgtgcccccccctgcccaccccctgcgccagcagcgccatcgagttccccggcggcaaggagacctggcagtggatcaaccccgtggacttccaggaggaggccagcctgcaggaggccctggtggtggagatgagctgggacaagggcgagaggaccgagcccctggagaagaccgagctgcccgagggcgcccccgagctggccctggacaccgagctgagcctggaggacggcgacaggtgcaaggccaagatgggtagcggcgccacaaatttcagcctgctgaaacaggccggcgacgtggaagagaaccctggacctatggattggatctggcggatcctgttccttgtgggagctgccacaggcgcccactctgagcagaagctgatctccgaagaggacctgatggaccccgacctggaaatcagagccgccttcctgcggcagagaaacacagccctgagaacagaggtggccgagctggaacaagaggtgcagcggctggaaaacgaggtgtcccagtacgagacaagatacggccctctcggcggaggaaaaggtggcggaggttctggcggaggcggaagtggtggtggcggatctatggatcctgacctcgagattagggccgcctttctgagacagcggaacaccgctctgaggaccgaagtggccgaactcgagcaagaagtccagagactggaaaatgaagtctctcagtacgaaacccgctacggacctcttggaggcggcaaaatcagcaccggccacctgaccagcgacaacaggggcgacatcggcctgctgctgggcatgatcgtgttcgccgtgatgctgagcatcctgagcctgatcggcatcttcaacaggagcttcaggaccggcatcaagaggaggatcctgctgctgatccccaagtggctgtacgaggacatccccaacatgaagaacagcaacgtggtgaagatgctgcaggagaacagcgagctgatgaacaacaacagcagcgagcaggtgctgtacgtggaccccatgatcaccgagatcaaggagatcttcatccccgagcacaagcccaccgactacaagaaggagaacaccggccccctggagaccagggactacccccagaacagcctgttcgacaacaccaccgtggtgtacatccccgacctgaacaccggctacaagccccagatcagcaacttcctgcccgagggcagccacctgagcaacaacaacgagatcaccagcctgaccctgaagccccccgtggacagcctggacagcggcaacaaccccaggctgcagaagcaccccaacttcgccttcagcgtgagcagcgtgaacagcctgagcaacaccatcttcctgggcgagctgagcctgatcctgaaccagggcgagtgcagcagccccgacatccagaacagcgtggaggaggagaccaccatgctgctggagaacgacagccccagcgagaccatccccgagcagaccctgctgcccgacgagttcgtgagctgcctgggcatcgtgaacgaggagctgcccagcatcaacacctacttcccccagaacatcctggagagccacttcaacaggatcagcctgctggagaagggcagcggcgccaccaactttagcctgctgaaacaagccggcgacgtggaagagaatcccggacctatggtgagcaagggcgaggagctgttcaccggggtggtgcccatcctggtcgagctggacggcgacgtaaacggccacaagttcagcgtccgcggcgaaggtgagggagatgccaccaacggcaagctgaccctgaagttcatctgcaccaccggcaagctgcccgtgccctggcccaccctcgtgaccaccttcggctacggcgtggcctgcttcagccgctaccccgaccacatgaagcagcacgacttcttcaagtccgccatgcccgaaggctacgtccaggagcgcaccatctctttcaaggacgacggtacctacaagacccgcgccgaggtgaagttcgagggcgacaccctggtgaaccgcatcgagctgaagggcatcgacttcaaggaggacggcaacatcctggggcacaagctggagtacaacttcaacagccactacgtctatatcacggccgacaagcagaagaactgcatcaaggctaacttcaagatccgccacaacgttgaggacggcagcgtgcagctcgccgaccactaccagcagaacacccccatcggcgacggccccgtgctgctgcccgacaaccactacctgagccatcagtccaagctgagcaaagaccccaacgagaagcgcgatcacatggtcctgctggagttcgtgaccgccgccgggattacacatggcatggacgagctgtacaagtaa

SEQ ID NO: 255 - protein, 1051 aa, synthetic construct
MDWIWRILFLVGAATGAHSDYKDDDDKMDPDLEIEAAFLERENTALETRVAELRQRVQRLRNRVSQYRTRYGPLGGGKGGGGSGGGGSGGGGSMDPDLEIEAAFLERENTALETRVAELRQRVQRLRNRVSQYRTRYGPLGGGKEECISLTRQYFTVTNVIIFFAFVLLLSGALAYCLALQLYVRRRKKLPSVLLFKKPSPFIFISQRPSPETQDTIHPLDEEAFLKVSPELKNLDLHGSTDSGFGSTKPSLQTEEPQFLLPDPHPQADRTLGNREPPVLGDSCSSGSSNSTDSGICLQEPSLSPSTGPTWEQQVGSNSRGQDDSGIDLVQNSEGRAGDTQGGSALGHHSPPEPEVPGEEDPAAVAFQGYLRQTRCAEEKATKTGCLEEESPLTDGLGPKFGRCLVDEAGLHPPALAKGYLKQDPLEMTLASSGAPTGQWNQPTEEWSLLALSSCSDLGISDWSFAHDLAPLGCVAAPGGLLGSFNSDLVTLPLISSLQSSEGSGATNFSLLKQAGDVEENPGPMDWIWRILFLVGAATGAHSEQKLISEEDLMDPDLEIRAAFLRQRNTALRTEVAELEQEVQRLENEVSQYETRYGPLGGGKGGGGSGGGGSGGGGSMDPDLEIRAAFLRQRNTALRTEVAELEQEVQRLENEVSQYETRYGPLGGGKEPVCEQTTHDETVPSWMVAVILMASVFMVCLALLGCFALLWCVYKKTKYAFSPRNSLPQHLKEFLGHPHHNTLLFFSFPLSDENDVFDKLSVIAEDSESGKQNPGDSCSLGTPPGQGPQSGSGATNFSLLKQAGDVEENPGPMVSKGEELFTGVVPILVELDGDVNGHKFSVRGEGEGDATNGKLTLKFICTTGKLPVPWPTLVTTFGYGVACFSRYPDHMKQHDFFKSAMPEGYVQERTISFKDDGTYKTRAEVKFEGDTLVNRIELKGIDFKEDGNILGHKLEYNFNSHYVYITADKQKNCIKANFKIRHNVEDGSVQLADHYQQNTPIGDGPVLLPDNHYLSHQSKLSKDPNEKRDHMVLLEFVTAAGITHGMDELYK

SEQ ID NO: 256 - DNA, 3156 bp, synthetic construct
  source 1..3156: mol_type = other DNA
atggattggatctggcggatcctgttccttgtgggagctgccacaggcgcccacagcgactacaaggacgacgacgacaagatggaccccgacctggaaatcgaggccgccttcctggaaagagagaacacagccctggaaaccagagtggccgagctgagacagagagtgcagcggctgagaaaccgggtgtcccagtacagaaccagatacggacctctcggcggaggaaaaggtggcggaggttctggcggaggcggctctggtggtggtggatctatggatcctgacctcgagattgaagccgcctttctcgagcgcgagaataccgctctcgagacaagagtggctgaactgcggcagagggtccagagactgaggaacagagtgtctcagtaccggacacgctacggccctcttggaggtggaaaagaggagtgcatcagcctgaccaggcagtacttcaccgtgaccaacgtgatcatcttcttcgccttcgtgctgctgctgagcggcgccctggcctactgcctggccctgcagctgtacgtgaggaggaggaagaagctgcccagcgtgctgctgttcaagaagcccagccccttcatcttcatcagccagaggcccagccccgagacccaggacaccatccaccccctggacgaggaggccttcctgaaggtgagccccgagctgaagaacctggacctgcacggcagcaccgacagcggcttcggcagcaccaagcccagcctgcagaccgaggagccccagttcctgctgcccgacccccacccccaggccgacaggaccctgggcaacagggagccccccgtgctgggcgacagctgcagcagcggcagcagcaacagcaccgacagcggcatctgcctgcaggagcccagcctgagccccagcaccggccccacctgggagcagcaggtgggcagcaacagcaggggccaggacgacagcggcatcgacctggtgcagaacagcgagggcagggccggcgacacccagggcggcagcgccctgggccaccacagcccccccgagcccgaggtgcccggcgaggaggaccccgccgccgtggccttccagggctacctgaggcagaccaggtgcgccgaggagaaggccaccaagaccggctgcctggaggaggagagccccctgaccgacggcctgggccccaagttcggcaggtgcctggtggacgaggccggcctgcacccccccgccctggccaagggctacctgaagcaggaccccctggagatgaccctggccagcagcggcgcccccaccggccagtggaaccagcccaccgaggagtggagcctgctggccctgagcagctgcagcgacctgggcatcagcgactggagcttcgcccacgacctggcccccctgggctgcgtggccgcccccggcggcctgctgggcagcttcaacagcgacctggtgaccctgcccctgatcagcagcctgcagagcagcgagggtagcggcgccacaaatttcagcctgctgaaacaggccggcgacgtggaagagaaccctggacctatggattggatctggcggatcctgttccttgtgggagctgccacaggcgcccactctgagcagaagctgatctccgaagaggacctgatggaccccgacctggaaatcagagccgccttcctgcggcagagaaacacagccctgagaacagaggtggccgagctggaacaagaggtgcagcggctggaaaacgaggtgtcccagtacgagacaagatacggccctctcggcggaggaaaaggtggcggaggttctggcggaggcggaagtggtggtggcggatctatggatcctgacctcgagattagggccgcctttctgagacagcggaacaccgctctgaggaccgaagtggccgaactcgagcaagaagtccagagactggaaaatgaagtctctcagtacgaaacccgctacggacctcttggaggcggcaaagagcccgtgtgcgagcagaccacccacgacgagaccgtgcccagctggatggtggccgtgatcctgatggccagcgtgttcatggtgtgcctggccctgctgggctgcttcgccctgctgtggtgcgtgtacaagaagaccaagtacgccttcagccccaggaacagcctgccccagcacctgaaggagttcctgggccacccccaccacaacaccctgctgttcttcagcttccccctgagcgacgagaacgacgtgttcgacaagctgagcgtgatcgccgaggacagcgagagcggcaagcagaaccccggcgacagctgcagcctgggcaccccccccggccagggcccccagagcggcagcggcgccaccaactttagcctgctgaaacaagccggcgacgtggaagagaatcccggacctatggtgagcaagggcgaggagctgttcaccggggtggtgcccatcctggtcgagctggacggcgacgtaaacggccacaagttcagcgtccgcggcgaaggtgagggagatgccaccaacggcaagctgaccctgaagttcatctgcaccaccggcaagctgcccgtgccctggcccaccctcgtgaccaccttcggctacggcgtggcctgcttcagccgctaccccgaccacatgaagcagcacgacttcttcaagtccgccatgcccgaaggctacgtccaggagcgcaccatctctttcaaggacgacggtacctacaagacccgcgccgaggtgaagttcgagggcgacaccctggtgaaccgcatcgagctgaagggcatcgacttcaaggaggacggcaacatcctggggcacaagctggagtacaacttcaacagccactacgtctatatcacggccgacaagcagaagaactgcatcaaggctaacttcaagatccgccacaacgttgaggacggcagcgtgcagctcgccgaccactaccagcagaacacccccatcggcgacggccccgtgctgctgcccgacaaccactacctgagccatcagtccaagctgagcaaagaccccaacgagaagcgcgatcacatggtcctgctggagttcgtgaccgccgccgggattacacatggcatggacgagctgtacaagtaa

SEQ ID NO: 257 - protein, 1045 aa, synthetic construct
MDWIWRILFLVGAATGAHSDYKDDDDKMDPDLEIEAAFLERENTALETRVAELRQRVQRLRNRVSQYRTRYGPLGGGKGGGGSGGGGSGGGGSMDPDLEIEAAFLERENTALETRVAELRQRVQRLRNRVSQYRTRYGPLGGGKEFTFTTPKFAQGEIEAIVVPVCLAFLLTTLLGVLFCFNKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSISSSDENESSQNTSSTVQYSTVVHSGYRHQVPSVQVFSRSESTQPLLDSEERPEDLQLVDHVDGGDGILPRQQYFKQNCSQHESSPDISHFERSKQVSSVNEEDFVRLKQQISDHISQSCGSGQMKMFQEVSAADAFGPGTEGQVERFETVGMEAATDEGMPKSYLPQTVRQGGYMPQGSGATNFSLLKQAGDVEENPGPMDWIWRILFLVGAATGAHSEQKLISEEDLMDPDLEIRAAFLRQRNTALRTEVAELEQEVQRLENEVSQYETRYGPLGGGKGGGGSGGGGSGGGGSMDPDLEIRAAFLRQRNTALRTEVAELEQEVQRLENEVSQYETRYGPLGGGKPILRLHLPDNTLRWKVLPGILFLWGLFLLGCGLSLATSGRCYHLRHKVLPRWVWEKVPDPANSSSGQPHMEQVPEAQPLGDLPILEVEEMEPPPVMESSQPAQATAPLDSGYEKHFLPTPEELGLLGPPRPQVLAESGKQNPGDSCSLGTPPGQGPQSGSGATNFSLLKQAGDVEENPGPMVSKGEELFTGVVPILVELDGDVNGHKFSVRGEGEGDATNGKLTLKFICTTGKLPVPWPTLVTTFGYGVACFSRYPDHMKQHDFFKSAMPEGYVQERTISFKDDGTYKTRAEVKFEGDTLVNRIELKGIDFKEDGNILGHKLEYNFNSHYVYITADKQKNCIKANFKIRHNVEDGSVQLADHYQQNTPIGDGPVLLPDNHYLSHQSKLSKDPNEKRDHMVLLEFVTAAGITHGMDELYK

SEQ ID NO: 258 - DNA, 3069 bp, synthetic construct
  source 1..3069: mol_type = other DNA
atggattggatctggcggatcctgttccttgtgggagctgccacaggcgcccacagcgactacaaggacgacgacgacaagatggaccccgacctggaaatcgaggccgccttcctggaaagagagaacacagccctggaaaccagagtggccgagctgagacagagagtgcagcggctgagaaaccgggtgtcccagtacagaaccagatacggacctctcggcggaggaaaaggtggcggaggttctggcggaggcggctctggtggtggtggatctatggatcctgacctcgagattgaagccgcctttctcgagcgcgagaataccgctctcgagacaagagtggctgaactgcggcagagggtccagagactgaggaacagagtgtctcagtaccggacacgctacggccctcttggaggtggaaaagagttcaccttcaccacccccaagttcgcccagggcgagatcgaggccatcgtggtgcccgtgtgcctggccttcctgctgaccaccctgctgggcgtgctgttctgcttcaacaagagggacctgatcaagaagcacatctggcccaacgtgcccgaccccagcaagagccacatcgcccagtggagcccccacaccccccccaggcacaacttcaacagcaaggaccagatgtacagcgacggcaacttcaccgacgtgagcgtggtggagatcgaggccaacgacaagaagcccttccccgaggacctgaagagcctggacctgttcaagaaggagaagatcaacaccgagggccacagcagcggcatcggcggcagcagctgcatgagcagcagcaggcccagcatcagcagcagcgacgagaacgagagcagccagaacaccagcagcaccgtgcagtacagcaccgtggtgcacagcggctacaggcaccaggtgcccagcgtgcaggtgttcagcaggagcgagagcacccagcccctgctggacagcgaggagaggcccgaggacctgcagctggtggaccacgtggacggcggcgacggcatcctgcccaggcagcagtacttcaagcagaactgcagccagcacgagagcagccccgacatcagccacttcgagaggagcaagcaggtgagcagcgtgaacgaggaggacttcgtgaggctgaagcagcagatcagcgaccacatcagccagagctgcggcagcggccagatgaagatgttccaggaggtgagcgccgccgacgccttcggccccggcaccgagggccaggtggagaggttcgagaccgtgggcatggaggccgccaccgacgagggcatgcccaagagctacctgccccagaccgtgaggcagggcggctacatgccccagggtagcggcgccacaaatttcagcctgctgaaacaggccggcgacgtggaagagaaccctggacctatggattggatctggcggatcctgttccttgtgggagctgccacaggcgcccactctgagcagaagctgatctccgaagaggacctgatggaccccgacctggaaatcagagccgccttcctgcggcagagaaacacagccctgagaacagaggtggccgagctggaacaagaggtgcagcggctggaaaacgaggtgtcccagtacgagacaagatacggccctctcggcggaggaaaaggtggcggaggttctggcggaggcggaagtggtggtggcggatctatggatcctgacctcgagattagggccgcctttctgagacagcggaacaccgctctgaggaccgaagtggccgaactcgagcaagaagtccagagactggaaaatgaagtctctcagtacgaaacccgctacggacctcttggaggcggcaaacccatcctgaggctgcacctgcccgacaacaccctgaggtggaaggtgctgcccggcatcctgttcctgtggggcctgttcctgctgggctgcggcctgagcctggccaccagcggcaggtgctaccacctgaggcacaaggtgctgcccaggtgggtgtgggagaaggtgcccgaccccgccaacagcagcagcggccagccccacatggagcaggtgcccgaggcccagcccctgggcgacctgcccatcctggaggtggaggagatggagcccccccccgtgatggagagcagccagcccgcccaggccaccgcccccctggacagcggctacgagaagcacttcctgcccacccccgaggagctgggcctgctgggcccccccaggccccaggtgctggccggcagcggcgccaccaactttagcctgctgaaacaagccggcgacgtggaagagaatcccggacctatggtgagcaagggcgaggagctgttcaccggggtggtgcccatcctggtcgagctggacggcgacgtaaacggccacaagttcagcgtccgcggcgaaggtgagggagatgccaccaacggcaagctgaccctgaagttcatctgcaccaccggcaagctgcccgtgccctggcccaccctcgtgaccaccttcggctacggcgtggcctgcttcagccgctaccccgaccacatgaagcagcacgacttcttcaagtccgccatgcccgaaggctacgtccaggagcgcaccatctctttcaaggacgacggtacctacaagacccgcgccgaggtgaagttcgagggcgacaccctggtgaaccgcatcgagctgaagggcatcgacttcaaggaggacggcaacatcctggggcacaagctggagtacaacttcaacagccactacgtctatatcacggccgacaagcagaagaactgcatcaaggctaacttcaagatccgccacaacgttgaggacggcagcgtgcagctcgccgaccactaccagcagaacacccccatcggcgacggccccgtgctgctgcccgacaaccactacctgagccatcagtccaagctgagcaaagaccccaacgagaagcgcgatcacatggtcctgctggagttcgtgaccgccgccgggattacacatggcatggacgagctgtacaagtaa

SEQ ID NO: 259 - protein, 1276 aa, synthetic construct
MDWIWRILFLVGAATGAHSDYKDDDDKMDPDLEIEAAFLERENTALETRVAELRQRVQRLRNRVSQYRTRYGPLGGGKGGGGSGGGGSGGGGSMDPDLEIEAAFLERENTALETRVAELRQRVQRLRNRVSQYRTRYGPLGGGKPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLVSDPVIFQTQSEELKEGWNPHLLLLLLLVIVFIPAFWSLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQASGSGATNFSLLKQAGDVEENPGPMDWIWRILFLVGAATGAHSEQKLISEEDLMDPDLEIRAAFLRQRNTALRTEVAELEQEVQRLENEVSQYETRYGPLGGGKSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPETGSGATNFSLLKQAGDVEENPGPMVSKGEELFTGVVPILVELDGDVNGHKFSVRGEGEGDATNGKLTLKFICTTGKLPVPWPTLVTTFGYGVACFSRYPDHMKQHDFFKSAMPEGYVQERTISFKDDGTYKTRAEVKFEGDTLVNRIELKGIDFKEDGNILGHKLEYNFNSHYVYITADKQKNCIKANFKIRHNVEDGSVQLADHYQQNTPIGDGPVLLPDNHYLSHQSKLSKDPNEKRDHMVLLEFVTAAGITHGMDELYK

SEQ ID NO: 260 - DNA, 4030 bp, synthetic construct
  source 1..4030: mol_type = other DNA
atggattggatctggcggatcctgttccttgtgggagctgccacaggcgcccacagcgactacaaggacgacgacgacaagatggaccccgacctggaaatcgaggccgccttcctggaaagagagaacacagccctggaaaccagagtggccgagctgagacagagagtgcagcggctgagaaaccgggtgtcccagtacagaaccagatacggacctctcggcggaggaaaaggtggcggaggttctggcggaggcggctctggtggtggtggatctatggatcctgacctcgagattgaagccgcctttctcgagcgcgagaataccgctctcgagacaagagtggctgaactgcggcagagggtccagagactgaggaacagagtgtctcagtaccggacacgctacggccctcttggaggtggaaaacctctggccttcagaacaaaacctgccgctctgggcaaagacacaatcccctggctgggacatctgctcgttggactgtctggcgccttcggcttcatcatcctggtgtacctgctgatcaactgccggaacacaggcccttggctgaagaaagtgctgaagtgcaacacccctgatccgagcaagttctttagccagctgagcagcgagcatggcggcgacgttcagaaatggctgtctagcccatttcctagcagcagcttcagccctggtggactggcccctgagattagccctctggaagtgctggaacgggacaaagtgacccagctgctcctgcagcaggataaggtgccagaacctgccagcctgtccagcaatcacagcctgaccagctgctttaccaaccagggctacttcttcttccatctgcctgacgctctcgagatcgaggcctgccaggtgtacttcacctacgatccctacagcgaggaagatcccgacgaaggcgttgcaggcgctcctacaggatcttctccacagcctctgcagccactgtccggcgaggatgatgcctactgcacctttccaagccgggacgacctgctgctgtttagccctagtctgctcggcggaccttctccaccatctacagctccaggtggaagcggagccggcgaagaaagaatgcctcctagcctgcaagagcgggtgcccagagattgggatcctcaacctctcggacctcctacaccaggcgtgccagacctggtggattttcagcctcctccagagctggtgctgagagaagctggcgaagaagtgccagacgccggacctagagagggcgttagctttccttggagcagacctcctggacagggcgagttcagagccctgaatgctagactgcccctgaacaccgatgcctacctgtctctgcaagaactgcagggacaagaccccacacacctcgttgagcgaccccgtgatctttcagacccagagcgaggaactgaaagaaggctggaaccctcacctcctgctgttgctgctgctggtcatcgtgttcatccccgccttttggagcctgaaaacacaccctctgtggcggctgtggaagaaaatctgggccgtgccatctcctgagcggttcttcatgcctctgtacaagggctgcagcggcgacttcaagaaatgggtcggagccccttttaccggcagctctctggaacttggaccttggagccctgaggtgcccagcacactggaagtgtacagctgtcaccctcctagaagccccgccaagagactgcagctgacagagctgcaagagcctgccgagctggtggaatctgatggcgtgcccaagcctagcttctggcccacagctcagaatagcggcggctctgcctacagcgaggaaagagatagaccctacggcctggtgtccatcgacaccgtgacagtgctggatgccgagggaccttgtacctggccttgtagctgcgaggacgatggctaccctgctctggatctggacgctggccttgagccttctccaggactggaagatcctctgctggacgccggaacaaccgtgctgtcttgtggctgtgtgtctgccggatctcctggacttggaggccctctgggaagcctgctggatagactgaaacctcctctggccgacggcgaagattgggctggtggacttccttggggcggaagatctccaggcggagtgtctgagtctgaagccggttctccactggccggcctggacatggataccttcgattctggcttcgtgggcagcgactgtagcagccctgtggaatgcgacttcacaagccctggcgacgagggcccacctagaagctatctgagacagtgggtcgtgatccctccacctctgtctagtcctggacctcaggcttctggtagcggcgccacaaatttcagcctgctgaaacaggccggcgacgtggaagagaaccctggacctatggattggatctggcggatcctgttccttgtgggagctgccacaggcgcccactctgagcagaagctgatctccgaagaggacctgatggaccccgacctggaaatcagagccgccttcctgcggcagagaaacacagccctgagaacagaggtggccgagctggaacaagaggtgcagcggctggaaaacgaggtgtcccagtacgagacaagatacggccctctcggcggaggaaaaggtggcggaggttctggcggaggcggaagtggtggtggcggatctatggatcctgacctcgagattagggccgcctttctgagacagcggaacaccgctctgaggaccgaagtggccgaactcgagcaagaagtccagagactggaaaatgaagtctctcagtacgaaacccgctacggacctcttggaggcggcaaaagcaacaccagcaaagagaaccccttcctgttcgccctggaagccgtggttatcagcgtgggctctatgggcctgatcatcagcctgctgtgcgtgtacttctggctggaacggaccatgcctcggatccccacactgaagaacctggaagatctggtcaccgagtaccacggcaacttctctgcttggagcggcgtgtcaaaaggcctggccgaatctctgcagcccgactactctgagagactgtgcctggtgtctgagatccctcctaaaggcggagccctcggagaaggacctggtgcctctccatgtaaccagcacagcccttattgggcccctccttgctacaccctgaagcctgaaacaggcagcggcgccaccaactttagcctgctgaaacaagccggcgacgtggaagagaatcccggacctatggtgagcaagggcgaggagctgttcaccggggtggtgcccatcctggtcgagctggacggcgacgtaaacggccacaagttcagcgtccgcggcgaaggtgagggagatgccaccaacggcaagctgaccctgaagttcatctgcaccaccggcaagctgcccgtgccctggcccaccctcgtgaccaccttcggctacggcgtggcctgcttcagccgctaccccgaccacatgaagcagcacgacttcttcaagtccgccatgcccgaaggctacgtccaggagcgcaccatctctttcaaggacgacggtacctacaagacccgcgccgaggtgaagttcgagggcgacaccctggtgaaccgcatcgagctgaagggcatcgacttcaaggaggacggcaacatcctggggcacaagctggagtacaacttcaacagccactacgtctatatcacggccgacaagcagaagaactgcatcaaggctaacttcaagatccgccacaacgttgaggacggcagcgtgcagctcgccgaccactaccagcagaacacccccatcggcgacggccccgtgctgctgcccgacaaccactacctgagccatcagtccaagctgagcaaagaccccaacgagaagcgcgatcacatggtcctgctggagttcgtgaccgccgccgggattacacatggcatggacgagctgtacaagtaa

SEQ ID NO: 261 - protein, 71 aa, synthetic construct
DLERNRAAASRCRQKRKVWVQSLEKKAEDLSSLNGQLQSEVTLLRNEVAQLKQLLLAHKDCPVTAMQKKSG

SEQ ID NO: 262 - protein, 54 aa, synthetic construct
KRKLWVSSLEKKAEELTSQNIQLSNEVTLLRNEVAQLKQLLLAHKDCPVTALQK

SEQ ID NO: 263 - protein, 56 aa, synthetic construct
QRKVWVMSLEKKAEELTQTNMQLQNEVSMLKNEVAQLKQLLLTHKDCPITAMQKES

SEQ ID NO: 264 - protein, 84 aa, synthetic construct
CPEEDERKKRRRERNKIAAAKCRNKKKEKTECLQKESEKLESVNAELKAQIEELKNEKQHLIYMLNLHRPTCIVRAQNGRTPED

SEQ ID NO: 265 - protein, 64 aa, synthetic construct
NKTAATRYRQKKRAEQEALTGECKELEKKNEALKERADSLAKEIQYLKDLIEEVRKARGKKRVP

SEQ ID NO: 266 - protein, 62 aa, synthetic construct
KKGTRYRQKKRVEQEVLTGECKAVEKKNEALQERADSLAEEIQYMKDSIEEVCKARGKKRVL

SEQ ID NO: 267 - protein, 74 aa, synthetic construct
NRKQKKRDQNKSAALRYRQRKRAEGEALEGECQGLEARNRELKERAESVEREIQYVKDLLIEVYKARSQRTRSC

SEQ ID NO: 268 - protein, 59 aa, synthetic construct
ACQSRKKKKEYMLGLEARLKAALSENEQLKKENGTLKRQLDEVVSENQRLKVPSPKRRV

SEQ ID NO: 269 - protein, 60 aa, synthetic construct
CRLNRLKKKEYVMGLESRVRGLAAENQELRAENRELGKRVQALQEESRYLRAVLANETGL

SEQ ID NO: 270 - protein, 42 aa, synthetic construct
QKADTLHLESEDLEKQNAALRKEIKQLTEELKYFTSVLNSHE

SEQ ID NO: 271 - protein, 63 aa, synthetic construct
RSRKKQTQKADKLHEEYESLEQENTMLRREIGKLTEELKHLTEALKEHEKMCPLLLCPMNFVH

SEQ ID NO: 272 - protein, 41 aa, synthetic construct
QRNVETQQKVLELTSDNDRLRKRVEQLSRELDTLRGIFRQL

SEQ ID NO: 273 - protein, 75 aa, synthetic construct
SDEYKIRRERNNIAVRKSRDKAKMRNLETQHKVLELTAENERLQKKVEQLSRELSTLRNLFKQLPEPLLASSGHC

SEQ ID NO: 274 - protein, 77 aa, synthetic construct
GERNNMAVKKSRLKSKQKAQDTLQRVNQLKEENERLEAKIKLLTKELSVLKDLFLEHAHNLADNVQSISTENTTADG

SEQ ID NO: 275 - protein, 41 aa, synthetic construct
RRNQEMQQKLVELSAENEKLHQRVEQLTRDLAGLRQFFKQL

SEQ ID NO: 276 - protein, 74 aa, synthetic construct
HLEYRLRRERNNIAVRKSRDKAKRRILETQQKVLEYMAENERLRSRVEQLTQELDTLRNLFRQIPEAANLIKGV

SEQ ID NO: 277 - protein, 51 aa, synthetic construct
RMKEKEQENERKVAQLAEENERLKQEIERLTREVEATRRALIDRMVNLHQA

SEQ ID NO: 278 - protein, 41 aa, synthetic construct
QRNVETQQKVLELTSDNDRLRNGVEQLSRELDTLRGIFRQL

SEQ ID NO: 279 - protein, 60 aa, synthetic construct
QRKLETIVQLERELERLTNERERLLRARGEADRTLEVMRQQLTELYRDIFQHLRDESGNS

SEQ ID NO: 280 - protein, 59 aa, synthetic construct
KLDTILNLERDVEDLQRDKARLLREKVEFLRSLRQMKQKVQSLYQEVFGRLRDENGRPY

SEQ ID NO: 281 - protein, 73 aa, synthetic construct
FRDIRRRGKNKVAAQNCRKRKLENIVELEQDLDHLKDEKEKLLKEKGENDKSLHLLKKQLSTLYLEVFSMLRD

SEQ ID NO: 282 - protein, 78 aa, synthetic construct
GDIRRRGKNKVAAQNCRKRKLDIILNLEDDVCNLQAKKETLKREQAQCNKAINIMKQKLHDLYHDIFSRLRDDQGRPV

SEQ ID NO: 283 - protein, 62 aa, synthetic construct
GCRKRKLDCIQNLESEIEKLQSEKESLLKERDHILSTLGETKQNLTGLCQKVCKEAALSQEQ

SEQ ID NO: 284 - protein, 55 aa, synthetic construct
RKRKLDCIQNLECEIRKLVCEKEKLLSERNQLKACMGELLDNFSCLSQEVCRDIQ

SEQ ID NO: 285 - protein, 61 aa, synthetic construct
DPQLKREIRLMKNREAARECRRKKKEYVKCLENRVAVLENQNKTLIEELKTLKDLYSNKSV

SEQ ID NO: 286 - protein, 63 aa, synthetic construct
KEEATRKRELRLMKNREAARECRRKKKEYVKCLENRVAVLENQNKTLIEELKALKDLYCHKVE

SEQ ID NO: 287 - protein, 59 aa, synthetic construct
KEEATRKRELRLMKNREAARECRRKKKEYVKCLENRVAVLENQNKTLIEELKALRDLYC

SEQ ID NO: 288 - protein, 32 aa, synthetic construct
EYVKCLESRVAVLEVQNKKLIEELETLKDICS

SEQ ID NO: 289 - protein, 62 aa, synthetic construct
KEAARKREVRLMKNREAARECRRKKKEYVKCLENRVAVLENQNKTLIEELKALKDLYCHKSD

SEQ ID NO: 290 - protein, 68 aa, synthetic construct
EKRRLNDLVLENKLIALGEENATLKAELLSLKLKFGLISSTAYAQEIQKLSNSTAVYFQDYQTSKSNV

SEQ ID NO: 291 - protein, 76 aa, synthetic construct
FRRERNKMAAAKCRNRRRELTDTLQAETDQLEDEKSALQTEIANLLKEKEKLEFILAAHRPACKIPDDLGFPEEMS

SEQ ID NO: 292 - protein, 68 aa, synthetic construct
NRERNKLAAAKCRNRRRELTDRLQAETDQLEEEKAELESEIAELQKEKERLEFVLVAHKPGCKIPYEA

SEQ ID NO: 293 - protein, 64 aa, synthetic construct
NRRKELTDFLQAETDKLEDEKSGLQREIEELQKQKERLELVLEAHRPICKIPEGAKEGDTGSTS

SEQ ID NO: 294 - protein, 64 aa, synthetic construct
ERNKLAAAKCRNRRRELTEKLQAETEELEEEKSGLQKEIAELQKEKEKLEFMLVAHGPVCKISP

SEQ ID NO: 295 - protein, 58 aa, synthetic construct
QRKLERIARLEEKVKTLKAQNSELASTANMLREQVAQLKQKVMNHVNSGCQLMLTQQY

SEQ ID NO: 296 - protein, 81 aa, synthetic construct
NIKVERKRLRNRLAATKCRKRKLERIARLEDKVKTLKAENAGLSSTAGLLREQVAQLKQKVMTHVSNGCQLLLGVKGHAFY

SEQ ID NO: 297 - protein, 72 aa, synthetic construct
DMDTQERIKAERKRLRNRIAASKCRKRKLERISRLEEKVKTLKSQNTELASTASLLREQVAQLKQKVLSHVN

SEQ ID NO: 298 - protein, 66 aa, synthetic construct
QRVQQRHVLESEKNQLLQQVDHLKQEISRLVRERDAYKEKYEKLVSSGFRENGSSSDNPSSPEFFM

SEQ ID NO: 299 - protein, 68 aa, synthetic construct
QYKRVQQKHHLENEKTQLIQQVEQLKQEVSRLARERDAYKVKCEKLANSGFREAGSTSDSPSSPEFFL

SEQ ID NO: 300 - protein, 51 aa, synthetic construct
EYVECLEKKVETFTSENNELWKKVETLENANRTLLQQLQKLQTLVTNKISR

SEQ ID NO: 301 - protein, 50 aa, synthetic construct
EYIDGLETRMSACTAQNQELQRKVLHLEKQNLSLLEQLKKLQAIVVQSTS

SEQ ID NO: 302 - protein, 73 aa, synthetic construct
WRRKIRNKRSAQESRRKKKVYVGGLESRVLKYTAQNMELQNKVQLLEEQNLSLLDQLRKLQAMVIEISNKTSS

SEQ ID NO: 303 - protein, 78 aa, synthetic construct
WRKIRNKQSAQDSRRRKKEYIDGLESRVAACSAQNQELQKKVQELERHNISLVAQLRQLQTLIAQTSNKAAQTSTCVL

SEQ ID NO: 304 - protein, 75 aa, synthetic construct
EEQKDEKYWSRRYKNNEAAKRSRDARRLKENQISVRAAFLEKENALLRQEVVAVRQELSHYRAVLSRYQAQHGAL

SEQ ID NO: 305 - protein, 45 aa, synthetic construct
LKENQIAIRASFLEKENSALRQEVADLRKELGKCKNILAKYEARH

SEQ ID NO: 306 - protein, 68 aa, synthetic construct
NWTRRKKNNVAAKRSRDARRLKENQITIRAAFLEKENTALRTEVAELRKEVGKCKTIVSKYETKYGPL

SEQ ID NO: 307 - protein, 89 aa, synthetic construct
GVTQKEELEKQKAELQQEVEKLASENASMKLELDALRSKYEALQTFARTVARSPVAPARGPLAAGLGPLVPGKVAATSVITIVKSKTDA

SEQ ID NO: 308 - protein, 50 aa, synthetic construct
TQKEELERQRVELQQEVEKLARENSSMRLELDALRSKYEALQTFARTVAR

SEQ ID NO: 309 - protein, 71 aa, synthetic construct
QTARDRKKARMSELEQQVVDLEEENQKLLLENQLLREKTHGLVVENQELRQRLGMDALVAEEEAEAKGNEV

SEQ ID NO: 310 - protein, 55 aa, synthetic construct
PSQNAFRKRKLERLEELEKKEAQLTVTNDQIHILKKENELLHFMLRSLLTERNMP

SEQ ID NO: 311 - protein, 54 aa, synthetic construct
KRAAQNRSAQKAFRQRREKYIKNLEEKSKLFDGLMKENSELKKMIESLKSKLKE

SEQ ID NO: 312 - protein, 54 aa, synthetic construct
EKRTAQNRAAQRAFRERKERKMKELEKKVQSLESIQQQNEVEATFLRDQLITLD

SEQ ID NO: 313 - protein, 105 aa, synthetic construct
EDTPPNPKKEIEFPGGSPGRKRIDSEAKSRRTAQNRAAQRAFRDRKEAKMKSLQERVELLEQKDAQNKTTTDFLLCSLKSLLSEITKYRAKNSDDERILAFLDDL

SEQ ID NO: 314 - protein, 79 aa, synthetic construct
EDTPPNPKKEIEFQAKKKAQNRAAQKAFRERKEARMKELQDKLLESERNRQSLLKEIEELRKANTEINAENRLLLRSGN

SEQ ID NO: 315 - protein, 94 aa, synthetic construct
EDTPPNPKKEIEFPKRQNRDAQRAYRERKNNKLQVLEETIESLSKVVKNYETKLNRLQNELQAKESENHALKQKLETLTLKQASVPAQDPILQN

SEQ ID NO: 316 - protein, 81 aa, synthetic construct
QQNRDAQRAYRERRTTRIQVLEEKVEMLHNLVDDWQRKYKLLESEFSDTKENLQKSIALNNELQKALPLIVNTPFQQQPEN

SEQ ID NO: 317 - protein, 77 aa, synthetic construct
EDTPPNPKKEIEFGIRKKQKNFENMNKLQNLNTQINKLRDRIEQLNKENEFWKAKLNDINEIKSLKLLNDIKRRNMG

SEQ ID NO: 318 - protein, 71 aa, synthetic construct
EDTPPNPKKEIEFHPAALKRARNTEAARRSRARKLQRMKQLEDKVEELLSKNYHLENEVARLKKLVGDAAR

SEQ ID NO: 319 - protein, 75 aa, synthetic construct
EDTPPNPKKEIEFDDDEDLLIQSKKSHQKKKLKEKELESSIHELTEIAASLQKRIHTLETENKLLKNLVLSSGET

SEQ ID NO: 320 - protein, 377 aa, synthetic construct
MDPDLEIEAAFLERENTALETRVAELRQRVQRLRNRVSQYRTRYGPLGGGKPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 321 - protein, 20 aa, synthetic construct
MSYYHHHHHHLESTSLYKKA

SEQ ID NO: 322 - protein, 4 aa, synthetic construct
GSGS

SEQ ID NO: 323 - protein, 4 aa, synthetic construct
GSEF

SEQ ID NO: 324 - protein, 4 aa, synthetic construct
GSGR

SEQ ID NO: 325 - protein, 4 aa, synthetic construct
GFDD

SEQ ID NO: 326 - protein, 5 aa, synthetic construct
YLEII

SEQ ID NO: 327 - protein, 17 aa, synthetic construct
CSSNTQLSCTKWLIRGC

SEQ ID NO: 328 - protein, 16 aa, synthetic construct
SSNTQLSCTKWLIRGC

SEQ ID NO: 329 - protein, 6 aa, synthetic construct
STCRDL

SEQ ID NO: 330 - protein, 7 aa, synthetic construct
FSTCRDL

SEQ ID NO: 331 - protein, 11 aa, synthetic construct
LDLQRSMNRRY

SEQ ID NO: 332 - protein, 7 aa, synthetic construct
PAEIYES